(19) United States
(12) Patent Application Publication
(10) Pub. No.: US 2024/0252623 A1
(43) Pub. Date: Aug. 1, 2024

(54) HEPATITIS C VIRUS IMMUNOGENIC COMPOSITIONS AND METHODS OF USE THEREOF

(71) Applicant: The Governors of the University of Alberta, Edmonton (CA)

(72) Inventors: Michael Houghton, Danville, CA (US); John L. Law, Edmonton (CA); Abdolamir Landi, Edmonton (CA)

(21) Appl. No.: 18/529,858

(22) Filed: Dec. 5, 2023

Related U.S. Application Data
(63) Continuation of application No. PCT/CA2022/050946, filed on Jun. 14, 2022.
(60) Provisional application No. 63/210,722, filed on Jun. 15, 2021.
(60) Provisional application No. 63/212,877, filed on Jun. 21, 2021.
(60) Provisional application No. 62/323,434, filed on Apr. 15, 2016.
(60) Provisional application No. 63/345,799, filed on May 25, 2022.

(51) Int. Cl. A61K 39/29; A61K 39/00; C07K 14/005

(52) U.S. Cl. CPC A61K 39/29; C07K 14/005; A61K 2039/53; A61K 2039/70; C12N 2770/24222; C12N 2770/24234

(57) ABSTRACT

The present disclosure provides nucleic acids comprising nucleotide sequences encoding hepatitis C virus (HCV) E1 and/or E2 polypeptides and/or one or more T-cell epitope polypeptides. The nucleic acids can comprise one or more modifications. The nucleic acids can be self-amplifying. The present disclosure provides compositions comprising the nucleic acids. The present disclosure provides methods of inducing an immune response to HCV in an individual, comprising administering to the individual a composition of the present disclosure.

### CROSS-REFERENCE

This application is a continuation of PCT Application No. PCT/CA2022/050946 which claims the benefit of U.S. Provisional Patent Application No. 63/210,722, filed Jun. 15, 2021, U.S. Provisional Patent Application No. 63/212,877, filed Jun. 21, 2021, U.S. Provisional Patent Application No. 63/323,434, filed Mar. 24, 2022, and U.S. Provisional Patent Application No. 63/345,799, filed May 25, 2022, which applications are incorporated herein by reference in their entirety.

### INCORPORATION BY REFERENCE OF SEQUENCE LISTING PROVIDED AS A SEQUENCE LISTING XML FILE

A Sequence Listing is provided herewith as a XML file, “UALB-056CON_SEQ_LIST_February_2024.xml” created on Feb. 7, 2024, and having a size of 741,326 bytes. The contents of the Sequence Listing XML are incorporated by reference herein in their entirety.

### INTRODUCTION

Hepatitis C virus (HCV) is a blood-borne pathogen that is estimated to infect 150-200 million people worldwide. Infection by HCV may be non-symptomatic, and can be cleared by patients, sometimes without medical intervention. However, the majority of patients develop a chronic HCV infection, which may lead to liver inflammation, scarring, and even to liver failure or liver cancer. In the United States alone, over 3 million people have a chronic infection.

The HCV virion contains a positive-sense single stranded RNA genome of about 9.5 kb. The genome encodes a single polyprotein of 3,010 to 3,030 amino acids. The structural proteins comprise a core protein forming the viral nucleocapsid and two envelope glycoproteins, E1 and E2.

There is a need in the art for compositions and methods for inducing immune responses to HCV.

### SUMMARY

The present disclosure provides nucleic acids comprising nucleotide sequences encoding hepatitis C virus (HCV) E1 and/or E2 polypeptides and/or one or more T-cell epitope polypeptides. The nucleic acids can comprise one or more modifications. The nucleic acids can be self-amplifying. The present disclosure provides compositions comprising the nucleic acids. The present disclosure provides methods of inducing an immune response to HCV in an individual, comprising administering to the individual a composition of the present disclosure.

### BRIEF DESCRIPTION OF THE DRAWINGS

FIG. 1A-1J provide an amino acid sequence alignment of examples of the core-E1-E2 coding regions of an HCV genotype 1 virus, specifically representative HCV 1A, 1B and 1C genotypes. Genbank database sequences for the coding region core-E1-E2 were aligned using Geneious software v5.6.4. Numbering of amino acids is according to strain NP_671941 (H77). Consensus: SEQ ID NO:1; AVI1a129: SEQ ID NO:2; NP_671491 (H77): SEQ ID NO:3; EU155269: SEQ ID NO:4; EU781810: SEQ ID NO:5; EU781771: SEQ ID NO:6; AB250610: SEQ ID NO:7; EU781752: SEQ ID NO:8; EU781759: SEQ ID NO:9; EF407439: SEQ ID NO:10; EF407427: SEQ ID NO:11; EU362905: SEQ ID NO:11; EF407413: SEQ ID NO:11; EU781808: SEQ ID NO:12; EU78170: SEQ ID NO:13; AJ238799 (Con1): SEQ ID NO:14; AAK97744: SEQ ID NO:15; AF139594: SEQ ID NO:16; AF176573: SEQ ID NO:17; BAA19625: SEQ ID NO:18; BAA25076: SEQ ID NO:19; BAC54896: SEQ ID NO:20; BAD91386: SEQ ID NO:21; BAF46764: SEQ ID NO:22; BAG30950: SEQ ID NO:22; CAB41951: SEQ ID NO:23; AAK95832: SEQ ID NO:24; AAT69968: SEQ ID NO:25; and BAA03581: SEQ ID NO:26.

FIG. 2A-2I provide an alignment of amino acid sequences of the core-E1-E2 coding region of representative HCV 2A and HCV2B subtypes. Genbank database sequences for the coding region core-E1-E2 were aligned using Geneious software v5.6.4. The amino acid numbering depicted is in accordance with the common HCV strains: D00994 (HC-J6) for HCV 2A; ABO47639 (JFH1) and HPCJ8G-J8 (J8) for HCV2A and HCV2B, respectively. Consensus: SEQ ID NO: 27; D00994 (HC-J6): SEQ ID NO: 28; AB047639 (JFH1): SEQ ID NO:29; AB047645: SEQ ID NO:30; AF238482: SEQ ID NO:31; AY746460: SEQ ID NO:32; HPCPOLP: SEQ ID NO:28; NC_009823: SEQ ID NO:34; HPCJ8G HC-J8: SEQ ID NO:35; AY232730: SEQ ID NO:36; AY232747: SEQ ID NO:37; AB030907: SEQ ID NO38; and DQ430817: SEQ ID NO:39.

FIG. 3A-3C provide an amino acid sequence alignment of the core-E1-E2 coding region for representative HCV 3A, 3B and 3K genotypes. Genbank database sequences for the coding region core-E1-E2 were aligned using Geneious software v5.6.4. Consensus: SEQ ID NO:40; AVI3a177: SEQ ID NO:41; ADF97232(S52): SEQ ID NO:42; YP_0014696: SEQ ID NO:43; CAA54244: SEQ ID NO:44; AAC03058: SEQ ID NO:45; AAY29642: SEQ ID NO:46; ABD85062: SEQ ID NO:47; ABD85063: SEQ ID NO:47; ABD97104: SEQ ID NO:48; BAA06044: SEQ ID NO:49; BAA08372: SEQ ID NO:50; and BAA09890: SEQ ID NO:51.

FIG. 4 provides amino acid sequences of examples of T-cell epitope polypeptides (“TPs”) that can be encoded by an RNA molecule of the present disclosure (SEQ ID Nos:52-61, respectively).

FIG. 5A-5E provide schematic depictions of various embodiments of a nucleic acid(s) of the present disclosure.

FIG. 6A-6H provide: i) nucleotide sequences encoding exemplary nucleic acids depicted schematically in FIG. 5A (FIG. 6A-6D) (SEQ ID Nos:62 and 64-66, respectively); and ii) mRNA (FIG. 6E-6H) encoded by the nucleotide sequences provided in FIG. 6A-6D (SEQ ID Nos:71-74, respectively).

FIG. 7A-7D provide nucleotide sequences of exemplary nucleic acids depicted schematically in FIG. 5B (SEQ ID Nos:79 and 64-66, respectively).

FIG. 8A-8F provide schematic depictions of various embodiments of a nucleic acid(s) of the present disclosure.

FIG. 9A-9C provide nucleotide sequences of exemplary nucleic acids depicted schematically in FIG. 8A (SEQ ID Nos:80-82, respectively).

FIG. 10A-10B provide nucleotide sequences of exemplary nucleic acids depicted schematically in FIG. 8B (SEQ ID Nos:83-84, respectively).

FIG. 11A-11D provide nucleotide sequences of exemplary nucleic acids depicted schematically in FIG. 8C (SEQ ID Nos:85-88, respectively).

FIG. 12A-12C provide nucleotide sequences of exemplary nucleic acids depicted schematically in FIG. 8D (SEQ ID Nos:89-91, respectively).

FIG. 13 provides a nucleotide sequence of the exemplary nucleic acid depicted schematically in FIG. 8E (SEQ ID No: 92).

FIG. 14 provides a nucleotide sequence of the exemplary nucleic acid depicted schematically in FIG. 8F (SEQ ID No: 93).

FIG. 15A-15G provide examples of suitable internal ribosome entry sites (IRES) (SEQ ID Nos:94-100).

FIG. 16A-16B provide an amino acid sequence (FIG. 16A) of TP50-C and a nucleotide sequence (FIG. 16B) encoding TP50-C, optimized for expression in human cells (SEQ ID Nos:101 and 67, respectively).

FIG. 17A-17B provide an amino acid sequence (FIG. 17A) of TP35-NS3 and a nucleotide sequence (FIG. 17B) encoding TP35-NS3, optimized for expression in human cells (SEQ ID Nos:102 and 68, respectively).

FIG. 18A-18B provide an amino acid sequence (FIG. 18A) of TP27 and a nucleotide sequence (FIG. 18B) encoding TP27, optimized for expression in human cells (SEQ ID Nos:103 and 69, respectively).

FIG. 19A-19B provide an amino acid sequence (FIG. 19A) of TP48 and a nucleotide sequence (FIG. 19B) encoding TP48, optimized for expression in human cells (SEQ ID Nos:104 and 70, respectively).

FIG. 20A-20B provide an amino acid sequence of the core-E1-E2 coding region for HCV genotype 7a. Amino acid sequence for the coding region core-E1-E2 of genotype 7a (isolate QC69; Genbank: ABN05226.1; SEQ ID NO:105) is shown according to the numbering scheme of the reference strain, NP_671941 (H77).

FIG. 21A-21D provide nucleotide sequences, codon optimized for expression in human cells, encoding HCV core-E1-E2 of genotype 1a (FIG. 21A) (SEQ ID NO:106), genotype 1b (FIG. 21B) (SEQ ID NO:107), genotype 2a (FIG. 21C) (SEQ ID NO:108), and genotype 3a (FIG. 21D) (SEQ ID NO:109).

FIG. 22A-22H provide schematic depictions of various embodiments of nucleic acids of the present disclosure.

FIG. 23A-23C provide nucleotide sequences of nucleic acids depicted schematically in FIG. 22A (SEQ ID Nos:110-112, respectively).

FIG. 24A-24I provide nucleotide sequences of nucleic acids depicted schematically in FIG. 22A (SEQ ID Nos:113-121, respectively).

FIG. 25A-25L provide nucleotide sequences of nucleic acids depicted schematically in FIG. 22B (SEQ ID Nos:122-133, respectively).

FIG. 26A-26B provide a nucleotide sequence (FIG. 26A) encoding an HCV Core-E1-E2 polyprotein of genotype 4 (SEQ ID NO:134); and an amino acid sequence (FIG. 26B) of the encoded HCV Core-E1-E2 polyprotein (SEQ ID NO:135).

FIG. 27A-27B provide a nucleotide sequence (FIG. 27A) encoding an HCV Core-E1-E2 polyprotein of genotype 5 (SEQ ID NO:136); and an amino acid sequence (FIG. 27B) of the encoded HCV Core-E1-E2 polyprotein (SEQ ID NO:137).

FIG. 28A-28B provide a nucleotide sequence (FIG. 28A) encoding an HCV Core-E1-E2 polyprotein of genotype 6 (SEQ ID NO:138); and an amino acid sequence (FIG. 28B) of the encoded HCV Core-E1-E2 polyprotein (SEQ ID NO:139).

FIG. 29 provides the amino acid sequence of a TP fusion polypeptide (SEQ ID NO:140).

FIG. 30 provides a schematic depiction of an embodiments of nucleic acids of the present disclosure.

FIG. 31A-31C provide examples of nucleotide sequences encoding RNAs, as depicted in FIG. 30 (SEQ ID NOs:141, 144 and 146, respectively).

FIG. 32A-32C provide amino acid sequences of E1/E2, TP465, and TP156 polypeptides encoded by the nucleotide sequence set forth in FIG. 31A (SEQ ID NOs:148-150, respectively).

FIG. 33A-33C provide amino acid sequences of E1/E2, TP465, and TP48 polypeptides encoded by the nucleotide sequence set forth in FIG. 31B (SEQ ID NOs:151-153, respectively).

FIG. 34A-34C provide amino acid sequences of E1/E2, TP465, and TP65 polypeptides encoded by the nucleotide sequence set forth in FIG. 31C (SEQ ID NOs:154-156, respectively).

### DEFINITIONS

The term “hepatitis C virus” (“HCV”), as used herein, refers to any one of a number of different genotypes and isolates of hepatitis C virus. Thus, “HCV” encompasses any of a number of genotypes, subtypes, or quasispecies, of HCV, including, e.g., genotype 1, 2, 3, 4, 5, 6, 7, etc. and subtypes (e.g., 1a, 1b, 2a, 2b, 3a, 4a, 4c, etc.), and quasispecies. Representative HCV genotypes and isolates include: HCV-1, H77, J6, Con1, isolate 1, BK, EC1, EC10, HC-J2, HC-J5; HC-J6, HC-J7, HC-J8, HC-JT, HCT18, HCT27, HCV-476, HCV-KF, “Hunan”, “Japanese”, “Taiwan”, TH, type 1, type 1a, H77 type 1b, type 1c, type Id, type 1e, type 1f, type 10, type 2, type 2a, type 2b, type 2c, type 2d, type 2f, type 3, type 3a, type 3b, type 3g, type 4, type 4a, type 4c, type 4d, type 4f, type 4h, type 4k, type 5, type 5a, type 6, type 6a, and type 7a.

The terms “individual,” “host,” “subject,” and “patient” are used interchangeably herein, and refer to a mammal, including, but not limited to, non-human primates (e.g., simians), equines (e.g., horses), rodents (e.g., rats; mice), and humans.

As used herein, the term “isolated,” in reference to a nucleic acid, refers to a nucleic acid that is in an environment different from that in which the nucleic acid naturally occurs or in which the nucleic acid was synthesized. An isolated nucleic acid can be purified. By “purified” is meant a compound of interest (e.g., a nucleic acid) has been separated from components that accompany it in nature or that are found together with the nucleic acid during synthesis. “Purified” can also be used to refer to a nucleic acid separated from components that can accompany it during production of the nucleic acid (e.g., during synthesis in vitro, etc.). In some cases, a nucleic acid (or a mixture of nucleic acids) is substantially pure when the nucleic acid (or mixture of nucleic acids) is at least 60% or at least 75% by weight free from organic molecules (e.g., proteins) with which it is naturally associated or with which it is associated during production. In some cases, the nucleic acid is from 30% to 60% pure. In some cases, the nucleic acid (or mixture of nucleic acid) is at least 60%, at least 75%, at least 80%, at least 85%, at least 90%, at least 95%, or at least 99%, by weight, pure.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. In some cases, a polynucleotide is RNA. In some cases, a polynucleotide is DNA. A “polynucleotide” includes a nucleic acid that is incorporated into a viral vector or a bacterial vector. A “polynucleoside” is a polymeric form of nucleosides or any length, and may include chemically or biochemically modified, non-natural, or derivatized nucleosides.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term “polypeptide” includes glycosylated polypeptides.

The term “heterologous” refers to two components that are defined by structures derived from different sources. For example, where “heterologous” is used in the context of a polypeptide, the polypeptide includes operably linked amino acid sequences that can be derived from one or more different polypeptides, e.g., amino acid sequences that are not operably linked to the polypeptide in nature. As another example, where a composition comprises an HCV E1/E2 heterodimer and a “heterologous” polypeptide, the “heterologous polypeptide is a polypeptide other than HCV E1 or HCV E2. As another example, where a fusion polypeptide comprises: a) a T-cell epitope polypeptide (a “TP”); and b) a heterologous fusion partner polypeptide, the “heterologous fusion partner polypeptide” is one that is not found associated with the T-cell epitope polypeptide in nature. As another example, where “heterologous” is used in the context of a nucleic acid or nucleotide sequence, the nucleic acid includes operably linked nucleotide sequences that are not normally linked in nature. For example, an IRES can be heterologous to a nucleotide sequence encoding an HCV E1 and/or E2 polypeptide, where the IRES is from an organism (e.g., a virus) other than HCV.

A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well known to those of skill in the art. See, e.g., Pearson (1994) Methods Mol. Biol. 24: 307-331, herein incorporated by reference. Examples of groups of amino acids that have side chains with similar chemical properties, and that thus constitute conservative amino acid substitution groups, include: 1) aliphatic side chain-containing amino acids: glycine, alanine, valine, leucine and isoleucine; 2) aliphatic-hydroxyl side chain-containing amino acids: serine and threonine; 3) amino acids with amide-containing side chains: asparagine and glutamine; 4) aromatic side chain-containing amino acids: phenylalanine, tyrosine, and tryptophan; 5) amino acids with basic side chains: lysine, arginine, and histidine; 6) amino acids with acidic side chains: aspartate and glutamate, and 7) amino acids with sulfur-containing side chains: cysteine and methionine. Examples of conservative amino acid substitution groups are: valine-leucine-isoleucine, phenylalanine-tyrosine, lysine-arginine, alanine-valine, glutamate-aspartate, and asparagine-glutamine.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a nucleic acid” includes a plurality of such nucleic acid and reference to “the nucleoside base modification” includes reference to one or more nucleoside base modifications and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides nucleic acids comprising nucleotide sequences encoding hepatitis C virus (HCV) E1 and/or E2 polypeptides. The nucleic acids can comprise one or more modifications. The nucleic acids can be self-amplifying. The present disclosure provides compositions comprising the nucleic acids. The present disclosure provides methods of inducing an immune response to HCV in an individual, comprising administering to the individual a composition of the present disclosure.

### Nucleic Acid Compositions

The present disclosure provides compositions (e.g., immunogenic compositions) comprising one or more nucleic acids comprising nucleotide sequences encoding one or more of: i) an HCV E1 polypeptide; ii) an HCV E2 polypeptide; iii) an HCV E1/E2 heterodimer; iv) an HCV core polypeptide; and v) a T-cell epitope polypeptide (a polypeptide other than an HCV E1 or E2 polypeptide; also referred to as a “TP”) as described below. In some cases, the one or more nucleic acids are DNA molecules. In some cases, the one or more nucleic acids are RNA (e.g., mRNA) molecules.

In some cases, a nucleic acid composition of the present disclosure comprises: a) one or more RNA molecules; and b) one or more of: i) a lipid; ii) a polymer; iii) a peptide; iv) a viral-like particle; and v) a cationic nanoemulsion; where the one or more RNA molecules comprises a polypeptide-encoding region comprising one or more nucleotide sequences encoding an HCV E1 polypeptide and/or an HCV E2 polypeptide. In some cases, a nucleic acid composition of the present disclosure comprises: a) one or more RNA molecules; and b) one or more of: i) a lipid; ii) a polymer; iii) a peptide; iv) a viral-like particle; and v) a cationic nanoemulsion; where the one or more RNA molecules comprises: i) a 5′ untranslated region (5′ UTR), e.g., a 5′ IRES; and ii) a polypeptide-encoding region comprising one or more nucleotide sequences encoding an HCV E1 polypeptide and/or an HCV E2 polypeptide. In some cases, a nucleic acid composition of the present disclosure comprises: a) one or more RNA molecules; and b) one or more of: i) a lipid; ii) a polymer; iii) a peptide; iv) a viral-like particle; and v) a cationic nanoemulsion; where the one or more RNA molecules comprises: i) a 5′ UTR; and ii) a polypeptide-encoding region comprising one or more nucleotide sequences encoding an HCV E1 polypeptide and/or an HCV E2 polypeptide. The one or more RNA molecules can further include additional polypeptide-encoding regions, as described below. The present disclosure provides nucleic acids comprising nucleotide sequences encoding HCV E1 and/or E2 polypeptides and/or one or more T-cell epitope polypeptides. In some cases, the one or more RNA molecules are mRNA. In some cases, the one or more RNA molecules are self-replicating RNA molecules.

In some cases, a nucleic acid composition of the present disclosure comprises: a) one or more RNA molecules; and b) one or more of: i) a lipid; ii) a polymer; iii) a peptide; iv) a viral-like particle; and v) a cationic nanoemulsion; where the one or more RNA molecules comprises: i) at least one 5′ cap structure; and ii) a polypeptide-encoding region comprising one or more nucleotide sequences encoding an HCV E1 polypeptide and/or an HCV E2 polypeptide. In some cases, a nucleic acid composition of the present disclosure comprises: a) one or more RNA molecules; and b) one or more of: i) a lipid; ii) a polymer; iii) a peptide; iv) a viral-like particle; and v) a cationic nanoemulsion; where the one or more RNA molecules comprises: i) at least one 5′ cap structure and/or a 5′ untranslated region (5′ UTR); and ii) a polypeptide-encoding region comprising one or more nucleotide sequences encoding an HCV E1 polypeptide and/or an HCV E2 polypeptide. In some cases, a nucleic acid composition of the present disclosure comprises: a) one or more RNA molecules; and b) one or more of: i) a lipid; ii) a polymer; iii) a peptide; iv) a viral-like particle; and v) a cationic nanoemulsion; where the one or more RNA molecules comprises: i) a 5′ UTR; and ii) a polypeptide-encoding region comprising one or more nucleotide sequences encoding an HCV E1 polypeptide and/or an HCV E2 polypeptide. The one or more RNA molecules can further include additional polypeptide-encoding regions, as described below. The present disclosure provides nucleic acids comprising nucleotide sequences encoding HCV E1 and/or E2 polypeptides and/or one or more T-cell epitope polypeptides. In some cases, the one or more RNA molecules are mRNA. In some cases, the one or more RNA molecules are self-replicating RNA molecules.

In some cases, a nucleic acid composition of the present disclosure comprises: a) one or more RNA molecules; and b) a polyalkyleneimine; where the one or more RNA molecules comprises: i) at least one 5′ cap structure and/or a 5′ UTR; and a polypeptide-encoding region comprising one or more nucleotide sequences encoding an HCV E1 polypeptide and/or an HCV E2 polypeptide. The one or more RNA molecules can further include additional polypeptide-encoding regions, as described below. In some cases, the one or more RNA molecules are self-replicating RNA molecules.

### 5′ Cap Structure

As noted above, an RNA of the present disclosure in some cases comprises a 5′ cap structure. The 5′ cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

RNA molecules (e.g., mRNA molecules) may be 5′-end capped, generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. The ribose sugars of the terminal and/or anteterminal transcribed nucleotides of the 5′ end of the mRNA may optionally also be 2′-O-methylated.

Additional modifications include, but are not limited to, 2′-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs may be chemically (i.e., non-enzymatically) or enzymatically synthesized and/or linked to a nucleic acid molecule. For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m.sup.7G-3′mppp-G; which may equivalently be designated 3′ O-Me-m7G(5′)ppp(5′)G). The 3′-O atom of the other, unmodified, guanine becomes linked to the 5′-terminal nucleotide of the capped nucleic acid molecule (e.g., an mRNA or mmRNA). The N7- and 3′-O-methlyated guanine provides the terminal moiety of the capped RNA molecule.

Another exemplary cap is mCAP, which is similar to ARCA but has a 2′-O-methyl group on guanosine (i.e., N7,2′-O-dimethyl-guanosine-5′-triphosphate-5′-guanosine, m.sup.7Gm-ppp-G).

While cap analogs allow for the concomitant capping of a nucleic acid molecule in an in vitro transcription reaction, up to 20% of transcripts can remain uncapped. This, as well as the structural differences of a cap analog from an endogenous 5′-cap structures of nucleic acids produced by the endogenous, cellular transcription machinery, may lead to reduced translational competency and reduced cellular stability.

An RNA molecule of the present disclosure may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present invention are those which, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N, pN2p (cap 0), 7mG(5′)ppp(5′)NlmpNp (cap 1), and 7mG(5′)-ppp(5′)NlmpN2mp (cap 2).

A 5′ terminal cap suitable for inclusion in an RNA molecule of the present disclosure can include endogenous caps or cap analogs. A 5′ terminal cap suitable for inclusion in an RNA molecule of the present disclosure can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

In some cases, the 5′ cap structure present in an RNA molecule of the present disclosure is selected from the group consisting of Cap0, Cap1, ARCA, inosine, N1-methyl-guanosine, 2′fluoro-guanosine, 7-deaza-guanosine, 8-oxo-guanosine, 2-amino-guanosine, LNA-guanosine, and 2-azido-guanosine.

### 5′ UTR

An RNA molecule of the present disclosure can include a 5′ UTR. Natural 5′UTRs bear features which play roles in for translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTR also have been known to form secondary structures which are involved in elongation factor binding.

By engineering the features typically found in abundantly expressed genes of specific target organs, one can enhance the stability and protein production of the polynucleotides, primary constructs or mRNA of the disclosure. For example, introduction of 5′ UTR of liver-expressed mRNA, such as albumin, serum amyloid A, Apolipoprotein A/B/E, transferrin, alpha fetoprotein, erythropoietin, or Factor VIII, could be used to enhance expression of an RNA molecule, in hepatic cell lines or liver. Likewise, use of 5′ UTR from other tissue-specific mRNA to improve expression in that tissue is possible for muscle (MyoD, Myosin, Myoglobin, Myogenin, Herculin), for endothelial cells (Tie-1, CD36), for myeloid cells (C/EBP, AML1, G-CSF, GM-CSF, CD11b, MSR, Fr-1, i-NOS), for leukocytes (CD45, CD18), for adipose tissue (CD36, GLUT4, ACRP30, adiponectin) and for lung epithelial cells (SP-A/B/C/D).

Other non-UTR sequences may be incorporated into the 5′ (or 3′ UTR) UTRs. For example, introns or portions of introns sequences may be incorporated into the flanking regions of an RNA molecule of the present disclosure. Incorporation of intronic sequences may increase protein production as well as mRNA levels.

The following are non-limiting examples of suitable 5′ UTR sequences. Variants may be used, in which one or more nucleotides are added to, or removed from, the termini.

5′UTR #1:
(SEQ ID NO: 142)
GGGAAATAAGAGAGAAAAGA AGAGTAAGAAGAAATATAAG AGCCACC;
5′UTR #2:
(SEQ ID NO: 157)
GGGAGATCAGAGAGAAAAGA AGAGTAAGAAGAAATATAAG AGCCACC;
5′UTR #3:
(SEQ ID NO: 158)
GGAATAAAAGTCTCAACACA ACATATACAAAACAAACGAA
TCTCAAGCAATCAAGCATTC TACTTCTATTGCAGCAATTT AAATCATTTCTTTTAAAGCA
AAAGCAATTTTCTGAAAATT TTCACCATTTACGAACGATA GCAAC;
and
5′UTR #4:
(SEQ ID NO: 159)
GGGAGACAAGCUUGGCAUUCCGGUACUGUUGGUAAAGCCA CC.

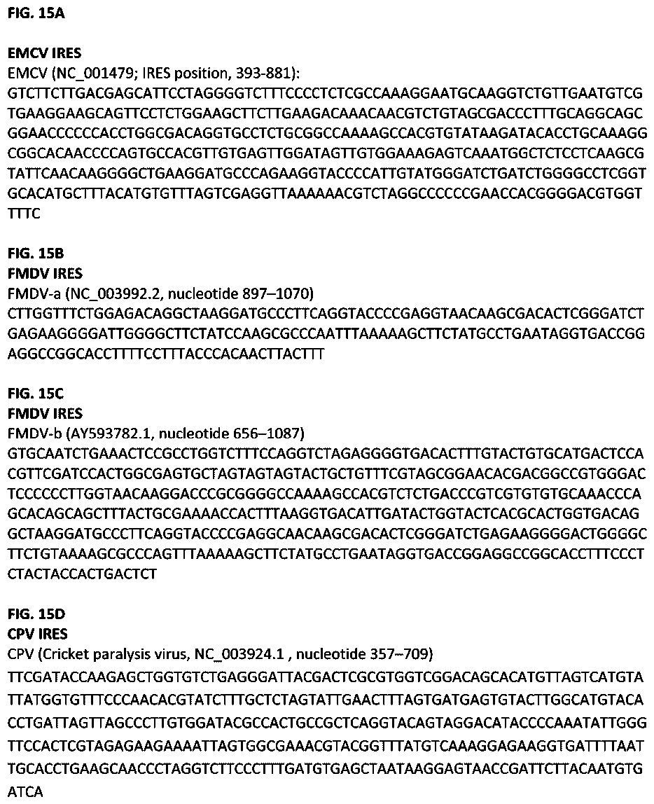

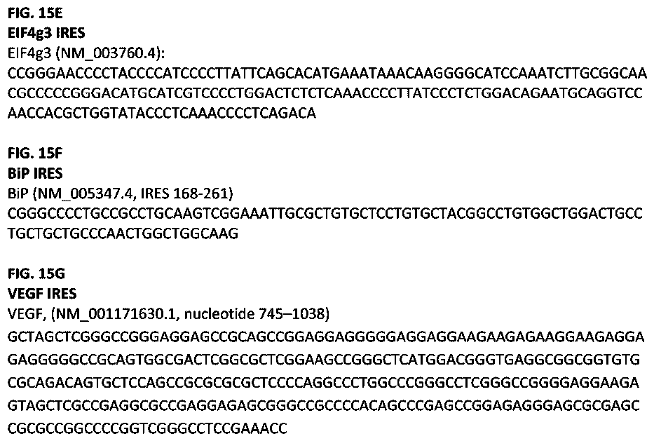

In some cases, the 5′ UTR is a 5′ IRES. Many IRES sequences are known in the art; any known IRES is suitable for use. Suitable 5′ IRESs are described below. Non-limiting examples of suitable IRESs are depicted in FIG. 15A-15G.

### 3′ UTR

An RNA molecule of the present disclosure can include a 3′ untranslated region (3′ UTR). 3′ UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes: Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo. Examples of suitable 3′ UTRs include those described in U.S. Pat. No. 9,827,332.

### Poly-A Tails

An RNA molecule of the present disclosure can include a poly(adenine) (poly-A) tail. The poly-A tail can be from 30 nucleotides to 250 nucleotides in length.

A nucleic acid (e.g., a DNA molecule) comprising a nucleotide sequence encoding RNA of the present disclosure can include a polyadenylation signal. Suitable polyadenylation signals are known in the art; and any such polyadenylation signals can be suitable for use. As is known in the art, a polyadenylation signal can include an AATAAA motif.

### RNA Modifications

An RNA molecule of the present disclosure can include one or more of: a) a modified nucleoside comprising a modified nucleoside base; b) a modified nucleoside comprising a modified sugar; c) a modified internucleoside linkage; and d) a modified backbone linking two or more nucleosides.

A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Suitable nucleic acid modifications include, but are not limited to: 2′O-methyl modified nucleotides, 2′ Fluoro modified nucleotides, locked nucleic acid (LNA) modified nucleotides, peptide nucleic acid (PNA) modified nucleotides, nucleotides with phosphorothioate linkages, and a 5′ cap (e.g., a 7-methylguanylate cap (m7G)). Additional details and additional modifications are described below.

A 2′-O-Methyl modified nucleoside (also referred to as 2′-O-Methyl RNA) is a naturally occurring modification of RNA found in tRNA and other small RNAs that arises as a post-transcriptional modification. RNA can be directly synthesized so that it contains one or more 2′-O-Methyl nucleosides. Such an RNA is stable with respect to attack by single-stranded ribonucleases.

2′ Fluoro modified nucleosides (e.g., 2′ Fluoro bases) have a fluorine modified ribose which increases binding affinity (Tm) and also confers some relative nuclease resistance when compared to native RNA.

LNA bases have a modification to the ribose backbone that locks the base in the C3′-endo position, which favors RNA A-type helix duplex geometry. This modification significantly increases Tm and is also very nuclease resistant. Multiple LNA insertions can be placed in an RNA at any position except the 3′-end.

The phosphorothioate (PS) bond (i.e., a phosphorothioate linkage) substitutes a sulfur atom for a non-bridging oxygen in the phosphate backbone of a nucleic acid (e.g., an oligo). This modification renders the internucleoside linkage resistant to nuclease degradation. Phosphorothioate bonds can be introduced between the last 3-5 nucleosides at the 5′- or 3′-end of an RNA molecule to inhibit exonuclease degradation. Including phosphorothioate bonds within an RNA molecule can help reduce attack by endonucleases as well.

In some cases, a subject nucleic acid has one or more nucleotides that are 2′-O-Methyl modified nucleosides. In some cases, a subject nucleic acid has one or more 2′ Fluoro modified nucleosides. In some cases, a subject nucleic acid has one or more LNA bases. In some cases, a subject nucleic acid has one or more nucleosides that are linked by a phosphorothioate bond (i.e., the subject nucleic acid has one or more phosphorothioate linkages).

### Modified Backbones and Modified Internucleoside Linkages

Examples of suitable nucleic acids (e.g., RNA molecules) containing modifications include nucleic acids containing modified backbones or non-natural internucleoside linkages. Nucleic acids having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleoside linkages is a 3′ to 3′, 5′ to 5′ or 2′ to 2′ linkage. Suitable RNA molecules having inverted polarity comprise a single 3′ to 3′ linkage at the 3′-most internucleoside linkage i.e. a single inverted nucleoside residue which may be a basic (the nucleobase is missing or has a hydroxyl group in place thereof). Various salts (such as, for example, potassium or sodium), mixed salts and free acid forms are also included.

In some cases, a subject nucleic acid comprises one or more phosphorothioate and/or heteroatom internucleoside linkages, in particular —CH₂—NHO—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleoside linkage is represented as —O—P(═O)(OH)—O—CH₂—). MMI type internucleoside linkages are disclosed in U.S. Pat. No. 5,489,677, the disclosure of which is incorporated herein by reference in its entirety. Suitable amide internucleoside linkages are disclosed in U.S. Pat. No. 5,602,240, the disclosure of which is incorporated herein by reference in its entirety.

Also suitable are nucleic acids having morpholino backbone structures as described in, e.g., U.S. Pat. No. 5,034,506. For example, in some cases, a subject nucleic acid comprises a 6-membered morpholino ring in place of a ribose ring. In some of these instances, a phosphorodiamidate or other non-phosphodiester internucleoside linkage replaces a phosphodiester linkage.

Suitable modified polynucleoside backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Mimetics

A subject nucleic acid can be a nucleic acid mimetic. The term “mimetic” as it is applied to polynucleotides is intended to include polynucleotides wherein only the furanose ring or both the furanose ring and the internucleoside linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleoside mimetic that has been shown to have excellent hybridization properties, is referred to as a peptide nucleic acid (PNA). In PNA, the sugar-backbone of a polynucleoside is replaced with an amide containing backbone, in particular an aminoethylglycine backbone. The nucleotides are retained and are bound directly or indirectly to aza nitrogen atoms of the amide portion of the backbone.

One polynucleoside mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262, the disclosures of which are incorporated herein by reference in their entirety.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41(14), 4503-4510). Morpholino-based polynucleotides are disclosed in U.S. Pat. No. 5,034,506, the disclosure of which is incorporated herein by reference in its entirety. A variety of compounds within the morpholino class of polynucleotides have been prepared, having a variety of different linking groups joining the monomeric subunits.

A further class of polynucleoside mimetic is referred to as cyclohexenyl nucleic acids (CeNA). The furanose ring normally present in an RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602, the disclosure of which is incorporated herein by reference in its entirety).

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂—), group bridging the 2′ oxygen atom and the 4′ carbon atom wherein n is 1 or 2 (Singh et al., Chem. Commun., 1998, 4, 455-456, the disclosure of which is incorporated herein by reference in its entirety). LNA and LNA analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (e.g., Wahlestedt et al., Proc. Natd. Acad. Sci. U.S.A., 2000, 97, 5633-5638, the disclosure of which is incorporated herein by reference in its entirety).

The synthesis and preparation of the LNA monomers adenine, cytosine, guanine, 5-methyl-cytosine, thymine and uracil, along with their oligomerization, and nucleic acid recognition properties have been described (e.g., Koshkin et al., Tetrahedron, 1998, 54, 3607-3630, the disclosure of which is incorporated herein by reference in its entirety). LNAs and preparation thereof are also described in WO 98/39352 and WO 99/14226, as well as U.S. applications 20120165514, 20100216983, 20090041809, 20060117410, 20040014959, 20020094555, and 20020086998, the disclosures of which are incorporated herein by reference in their entirety.

### Modified Sugar Moieties

A subject nucleic acid can also include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleosides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an RNA, or a group for improving the pharmacodynamic properties of an RNA, and other substituents having similar properties. A suitable modification includes 2′-methoxyethoxy (2′-O—CH₂ CH₂OCH₃, also known as 2′-O-(2-methoxyethyl) or 2′-MOE) (Martin et al., Helv. Chim. Acta, 1995, 78, 486-504, the disclosure of which is incorporated herein by reference in its entirety) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other suitable sugar substituent groups include methoxy (—O—CH₃), aminopropoxy (—OCH₂ CH₂ CH₂NH₂), allyl (—CH₂—CH═CH₂), —O-allyl (—O-CH₂—CH═CH₂) and fluoro (F). 2′-sugar substituent groups may be in the arabino (up) position or ribo (down) position. A suitable 2′-arabino modification is 2′-F. Similar modifications may also be made at other positions on the RNA, particularly the 3′ position of the sugar on the 3′ terminal nucleoside or in 2′-5′ linked oligonucleosides and the 5′ position of 5′ terminal nucleoside.

### Base Modifications and Substitutions

A subject nucleic acid may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C-CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993; the disclosures of which are incorporated herein by reference in their entirety. Certain of these nucleobases are useful for increasing the binding affinity of an oligomeric compound. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278; the disclosure of which is incorporated herein by reference in its entirety) and are suitable base substitutions, e.g., when combined with 2′-O-methoxyethyl sugar modifications.

### RNA Comprising Nucleosides of Formula I

In some cases, a subject RNA comprises n number of linked nucleosides of Formula I:

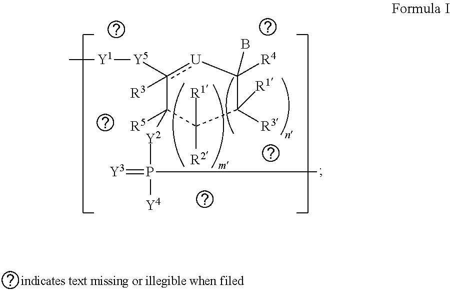

wherein:
U is O, S, N(R^{U})ₙᵤ, or C(R^{U})ₙᵤ, where nu is an integer nu is an integer from 0 to 2 and each R^{U} is, independently, H, halo, or optionally substituted alkyl; [symbol] is a single or double bond; [symbol] is a single bond or absent; each of R^{1′}, R^{2′}, R^{1″}, R^{2″}, R³, R⁴, and R⁵ is, independently, H, halo, hydroxy, thiol, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, or absent; wherein the combination of R³ with one or more of R^{1′}, R^{1″}, R^{2′}, R^{2″}, or R⁵ can join together to form optionally substituted alkylene or optionally substituted heteroalkylene and, taken together with the carbons to which they are attached, provide an optionally substituted heterocyclyl; wherein the combination of R⁵ with one or more of R^{1′}, R^{1″}, R^{2′}, or R^{2″} can join together to form optionally substituted alkylene or optionally substituted heteroalkylene and, taken together with the carbons to which they are attached, provide an optionally substituted heterocyclyl; and wherein the combination of R⁴ and one or more of R^{1′}, R^{1″}, R^{2′}, R^{2″}, R³, or R⁵ can join together to form optionally substituted alkylene or optionally substituted heteroalkylene and, taken together with the carbons to which they are attached, provide an optionally substituted heterocyclyl; each of Y¹, Y², and Y³, is, independently, O, S, —NR^{N1}—, optionally substituted alkylene, or optionally substituted heteroalkylene, wherein R^{N1} is H, optionally substituted alkyl, optionally substituted alkenyl, optionally substituted alkynyl, optionally substituted aryl, or absent; each Y⁴ is, independently, H, hydroxy, thiol, optionally substituted alkyl, optionally substituted alkenyl, optionally substituted alkynyl, optionally substituted alkoxy, optionally substituted alkenyloxy, optionally substituted alkynyloxy, optionally substituted thioalkoxy, optionally substituted alkoxyalkoxy, or optionally substituted amino; each Y⁵ is, independently, O, S, optionally substituted alkylene, or optionally substituted heteroalkylene; n is an integer from 1 to 100,000; and B is a nucleobase, wherein the combination of B and R^{1′}, the combination of B and R^{2′}, the combination of B and R^{1″}, or the combination of B and R^{2″} can, taken together with the carbons to which they are attached, optionally form a bicyclic group or wherein the combination of B, R^{1″}, and R³ or the combination of B, R^{2″}, and R³ can optionally form a tricyclic or tetracyclic group. In some cases, B is not pseudouridine or 5-methyl-cytidine. In some cases, B is pseudouridine. In some cases, B is 5-methyl-cytidine.

In some cases, U is O or C(R^{U})ₙᵤ, wherein nu is an integer from 1 to 2 and each R^{U} is, independently, H, halo, or optionally substituted alkyl; each of R¹, R^{1′}, R^{1″}, R², R^{2′}, and R^{2″}, if present, is, independently, H, halo, hydroxy, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted amino, azido, optionally substituted aryl, or optionally substituted aminoalkyl; each of R³ and R⁴ is, independently, H, halo, hydroxy, optionally substituted alkyl, or optionally substituted alkoxyalkoxy; each of Y¹, Y², and Y³, is, independently, O, S, —NR^{N1}—, optionally substituted alkylene, or optionally substituted heteroalkylene, wherein R^{N1} is H, optionally substituted alkyl, optionally substituted alkenyl, or optionally substituted alkynyl; each Y⁴ is, independently, H, hydroxy, thiol, optionally substituted alkyl, optionally substituted alkenyl, optionally substituted alkynyl, optionally substituted alkoxy, optionally substituted alkenyloxy, optionally substituted alkynyloxy, optionally substituted thioalkoxy, or optionally substituted amino; each Y⁵ is, independently, O or optionally substituted alkylene; and n is an integer from 10 to 10,000. In some cases, each of R¹, R^{1′}, and R^{1″}, if present, is H. In some cases, each of R², R^{2′}, and R^{2″}, if present, is, independently, H, halo, hydroxy, optionally substituted alkoxy, or optionally substituted alkoxyalkoxy. In some cases, each of R², R^{2′}, and R^{2″}, if present, is H. In some cases, each of R¹, R^{1′}, and R^{1″}, if present, is, independently, H, halo, hydroxy, optionally substituted alkoxy, or optionally substituted alkoxyalkoxy. In some cases, U is O or C(R^{U})n, where nu is an integer from 1 to 2 and each R^{U} is, independently, H, halo, or optionally substituted alkyl; each of R¹ and R² is independently H, halo, hydroxy, optionally substituted alkyl, optionally substituted alkoxy, optionally substituted alkoxyalkoxy, optionally substituted amino, azido, optionally substituted aryl, or optionally substituted aminoalkyl; each of R³ and R⁴ is, independently, H or optionally substituted alkyl; each of Y¹, Y², and Y³ is, independently O, S, —NR^{N1}—, optionally substituted alkylene, or optionally substituted heteroalkylene, wherein R^{N1} is H, optionally substituted alkyl, optionally substituted alkenyl, or optionally substituted alkynyl; each Y⁴ is, independently Y, hydroxy, thiol, optionally substituted alkyl, optionally substituted alkenyl, optionally substituted alkynyl, optionally substituted alkoxy, optionally substituted alkenyloxy, optionally substituted alkynyloxy, optionally substituted thioalkoxy, or optionally substituted amino; each Y⁵ is independently O or optionally substituted alkylene; and n is an integer from 10 to 10,000.

In some cases, a subject RNA comprises n number of linked nucleosides of Formula Ia:

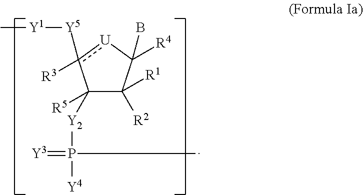

where the substituents are defined as for Formula I, above.

In some cases, a subject RNA comprises n number of linked nucleosides of Formula Ib:

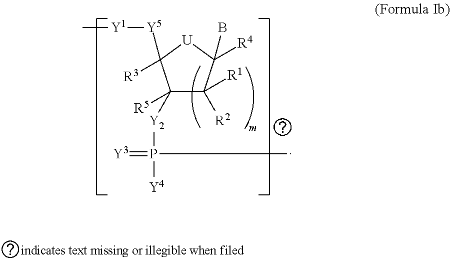

where the substituents are defined as for Formula I, above.

In some cases, n number of B is of Formula II, III, or IV,

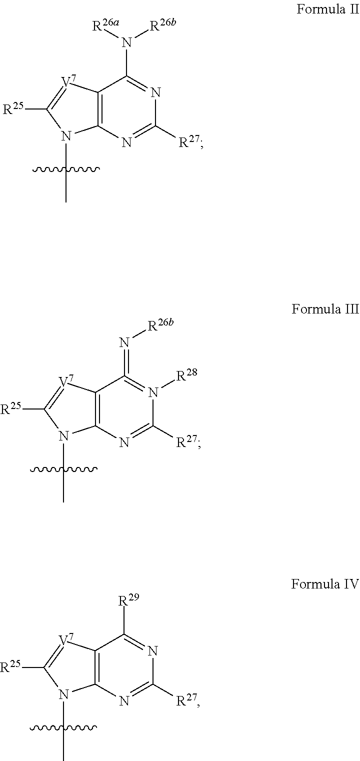

wherein each V⁷ is, independently, O, S, N(R^{Ve})ₙᵥ, or C(R^{Ve})ₙᵥ, wherein nv is an integer from 0 to 2 and each RV^{e} is, independently, H, halo, optionally substituted amino acid, optionally substituted alkyl, optionally substituted alkenyl, optionally substituted alkynyl, optionally substituted alkoxy, optionally substituted alkenyloxy, or optionally substituted alkynyloxy; each R²¹ is, independently, H, optionally substituted alkyl, optionally substituted alkenyl, or optionally substituted alkynyl; each of R^{26a} and R^{26b} is, independently, H, optionally substituted acyl, optionally substituted amino acid, optionally substituted carbamoylalkyl, optionally substituted alkyl, optionally substituted alkenyl, optionally substituted alkynyl, optionally substituted hydroxyalkyl, optionally substituted hydroxyalkenyl, or optionally substituted alkoxy; each R²⁷ is, independently, H, optionally substituted alkyl, optionally substituted alkenyl, optionally substituted alkynyl, optionally substituted alkoxy, or optionally substituted amino; each R²⁸ is, independently, H, optionally substituted alkyl, optionally substituted alkenyl, or optionally substituted alkynyl; and each R²⁹ is, independently, H, optionally substituted acyl, optionally substituted amino acid, optionally substituted carbamoylalkyl, optionally substituted alkyl, optionally substituted alkenyl, optionally substituted alkynyl, optionally substituted hydroxyalkyl, optionally substituted hydroxyalkenyl, optionally substituted alkoxy, or optionally substituted amino.

### Self-Amplifying RNA

In some cases, an RNA molecule of the present disclosure is a self-amplifying (also referred to herein as “self-replicating”) RNA.

A self-replicating RNA molecule (also referred to as a “replicon”) can, when delivered to a vertebrate cell even without any proteins, lead to the production of multiple daughter RNAs by transcription from itself (via an antisense copy which it generates from itself). A self-replicating RNA molecule is thus in certain cases, a (+) strand molecule that can be directly translated after delivery to a cell, and this translation provides an RNA-dependent RNA polymerase (RdRP) which then produces both antisense and sense transcripts from the delivered RNA. Thus, the delivered RNA leads to the production of multiple daughter RNAs. These daughter RNAs, as well as co-linear subgenomic transcripts, may be translated themselves to provide in situ expression of an encoded immunogen, or may be transcribed to provide further transcripts with the same sense as the delivered RNA which are translated to provide in situ expression of the immunogen. The overall result of this sequence of transcriptions is a large amplification in the number of the introduced replicon RNAs and so the encoded immunogen becomes a major polypeptide product of the host cells. For example, a self-amplifying RNA can include 5′ and 3′ conservative sequence elements (CSE), nsP1-4 genes, and a coding sequence encoding one or more antigenic polypeptides (e.g., one or more of HCV E1, HCV E2, a TP, a fusion protein comprising two or more TPs, etc.) where the coding sequence is operably linked to a promoter. Following translation in situ (e.g., in a cell in an individual), the nsP1-4 proteins form an RdRP complex that recognizes the 5′ CSE and 3′ CSE and amplifies the antigen-encoding RNAs.

One suitable system for achieving self-replication is to use an alphavirus-based RNA replicon. These (+) stranded replicons are translated after delivery to a cell to give of a replicase (or replicase-transcriptase). The replicase is translated as a polyprotein which auto cleaves to provide a replication complex which creates genomic (−) strand copies of the (+) strand delivered RNA. These (−) strand transcripts can themselves be transcribed to give further copies of the (+) stranded parent RNA and also to give a subgenomic transcript which encodes the immunogen. Translation of the subgenomic transcript thus leads to in situ expression of the immunogen by the infected cell. Suitable alphavirus replicons can use a replicase from a sindbis virus, a semliki forest virus, an eastern equine encephalitis virus, a Venezuelan equine encephalitis virus, etc. Mutant or wild-type viruses sequences can be used e.g. the attenuated TC83 mutant of VEEV has been used in replicons. Other RNA virus-based RNA replicons can be used, including those of positive-strand RNA viruses and negative-strand RNA viruses.

In some cases, self-replicating RNA molecule of the present disclosure encodes (i) a RdRP which can transcribe RNA from the self-replicating RNA molecule and (ii) a polypeptide-encoding region comprising nucleotide sequences encoding one or more nucleotide sequences encoding HCV E1 and/or E2 polypeptides and/or one or more T-cell epitope polypeptides. The RdRP of any RNA virus can be used. The RdRP can be an alphavirus replicase e.g., comprising one or more of alphavirus proteins nsP1, nsP2, nsP3 and nsP4. See, e.g., Geall et al. (2012) Proc. Natd. Acad. Sci. USA 109:14604.

Whereas natural alphavirus genomes encode structural virion proteins in addition to the non-structural replicase polyprotein, in some cases, a self-replicating RNA molecule of the present disclosure does not encode alphavirus structural proteins. Thus, e.g., a particular self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding an immunogen of interest (HCV E1 and/or E2 polypeptides; T-cell epitope polypeptides; etc.), such that the subgenomic transcript encodes the immunogen rather than the structural alphavirus virion proteins.

In some cases, a self-replicating RNA molecule of the present disclosure comprises two open reading frames. One open reading frame encodes a replicase (e.g., RdRP) e.g., the first, (5′) open reading frame; the other open reading frame encodes HCV E1 and/or E2 polypeptides (and in some cases additional polypeptides such as T-cell epitope polypeptides), e.g., the second, (3′) open reading frame. As another example, the first (5′) open reading frame encodes HCV E1 and/or E2 polypeptides (and in some cases additional polypeptides such as T-cell epitope polypeptides); and the second (3′) open reading frame encodes a replicase (e.g., RdRP).

A self-replicating RNA molecule of the present disclosure can have a 5′ sequence which is compatible with the encoded replicase. A self-replicating RNA molecule of the present disclosure can include a 5′ cap structure, where suitable 5′ cap structures are known in the art and are described above. The 5′ nucleotide of a self-replicating RNA molecule of the present disclosure can include a 5′ triphosphate group. A self-replicating RNA molecule of the present disclosure can include a 3′ poly-A tail. A self-replicating RNA molecule of the present disclosure can include a poly-A polymerase recognition sequence (e.g., AAUAAA) near its 3′ end. (in addition to any 5′ cap structure) one or more nucleotides having a modified nucleobase. For instance, a self-replicating RNA molecule of the present disclosure can include one or more modified pyrimidine nucleobases, such as pseudouridine and/or 5 methylcytosine residues. In some cases, however, the self-replicating RNA includes no modified nucleobases, and may include no modified nucleotides i.e., all of the nucleotides in the self-replicating RNA are standard A, C, G and U ribonucleotides (except for any 5′ cap structure, which may include a 7′ methylguanosine). In other instances, a self-replicating RNA of the present disclosure can include a 5′ cap comprising a 7′ methylguanosine, and the first 1, 2 or 3 5′ ribonucleotides may be methylated at the 2′ position of the ribose.

In some cases, a self-replicating RNA of the present disclosure is a recombinant alphavirus. An alphavirus found in nature is selected from the group consisting of the following: Barmah Forest virus complex (comprising Barmah Forest virus); Eastern equine encephalitis complex (comprising seven antigenic types of Eastern equine encephalitis virus); Middelburg virus complex (comprising Middelburg virus); Ndumu virus complex (comprising Ndumu virus); Semliki Forest virus complex (comprising Bebaru virus, Chikungunya virus, Mayaro virus and its subtype Una virus, O'Nyong Nyong virus, and its subtype Igbo-Ora virus, Ross River virus and its subtypes Bebaru virus, Getah virus, Sagiyama virus, Semliki Forest virus and its subtype Me Tri virus); Venezuelan equine encephalitis complex (comprising Cabassou virus, Everglades virus, Mosso das Pedras virus, Mucambo virus, Paramana virus, Pixuna virus, Rio Negro virus, Trocara virus and its subtype Bijou Bridge virus, Venezuelan equine encephalitis virus); Western equine encephalitis complex (comprising Aura virus, Babanki virus, Kyzylagach virus, Sindbis virus, Ockelbo virus, Whataroa virus, Buggy Creek virus, Fort Morgan virus, Highlands J virus, Western equine encephalitis virus); and some unclassified viruses including Salmon pancreatic disease virus; Sleeping Disease virus; Southern elephant seal virus; Tonate virus. More preferably, the alphavirus is selected from the group consisting of Semliki Forest virus complex (comprising the virus types as indicated above, including Semliki Forest virus), Western equine encephalitis complex (comprising the virus types as indicated above, including Sindbis virus), Eastern equine encephalitis virus (comprising the virus types as indicated above), Venezuelan equine encephalitis complex (comprising the virus types as indicated above, including Venezuelan equine encephalitis virus).

In some cases, the alphavirus is not an alphavirus found in nature. Typically, an alphavirus not found in nature is a variant or derivative of an alphavirus found in nature, that is distinguished from an alphavirus found in nature by at least one mutation in the nucleotide sequence, i.e., the genomic RNA. The mutation in the nucleotide sequence may be selected from an insertion, a substitution or a deletion of one or more nucleotides, compared to an alphavirus found in nature. A mutation in the nucleotide sequence may or may not be associated with a mutation in a polypeptide or protein encoded by the nucleotide sequence. For example, an alphavirus not found in nature may be an attenuated alphavirus. An attenuated alphavirus not found in nature is an alphavirus that typically has at least one mutation in its nucleotide sequence by which it is distinguished from an alphavirus found in nature, and that is either not infectious at all, or that is infectious but has a lower disease-producing ability or no disease-producing ability at all.

### IRES

In some cases, a nucleic acid of the present disclosure includes one or more (e.g., 1, 2, 3, 4, or 5, or more) internal ribosome entry sites (IRES). IRES elements are able to bypass the ribosome scanning model of 5′ methylated cap-dependent translation and begin translation at internal sites. Thus, an IRES provides a structure to which a ribosome can bind that does not need to be at the 5′ end of the mRNA. An IRES can therefore allow a ribosome to initiate translation at a second (or additional) initiation codon within a mRNA, allowing more than one polypeptide to be produced from a single mRNA.

Suitable IRESs include, but are not limited to, a picornavirus IRES, an Aphthovirus IRES, a Kaposi's sarcoma-associated herpesvirus IRES, a hepatitis A virus IRES, a hepatitis C virus IRES, a pestivirus IRES, a cripavirus IRES, a Friend murine leukemia virus IRES, a Moloney murine leukemia virus IRES, a Rouse sarcoma virus IRES, a human immunodeficiency virus IRES, a mammalian IRES, and the like. In some cases, the IRES is an IRES from a picornavirus (e.g. a pest viruses (CFFV), a polio virus (PV), an encephalomyocarditis virus (ECMV), a foot-and-mouth disease virus (FMDV), a hepatitis C virus (HCV), a classical swine fever virus (CSFV), a murine leukemia virus (MLV), a simian immune deficiency virus (SIV), or a cricket paralysis viruses (CPV). An IRES can be derived from a eukaryotic non-translated region, e.g., from BiP, fibroblast growth factor-1 (FGF-1), FGF-2, and the like.

In some cases, an RNA molecule of the present disclosure includes a single IRES. In some cases, an RNA molecule of the present disclosure includes more than one IRES. For example, in some cases, an RNA molecule of the present disclosure includes 2, 3, 4, or 5 IRESs. Where an RNA molecule of the present disclosure includes 2 or more IRESs, in some cases, the 2 or more IRESs are the same (have the same sequence). Where an RNA molecule of the present disclosure includes 2 or more IRESs, in some cases, the 2 or more IRESs have different sequences.

Non-limiting examples of suitable IRESs are depicted in FIG. 15A-15G.

### Self-Cleaving Peptides

In some cases, instead of an IRES, an RNA molecule of the present disclosure comprises a nucleotide sequence encoding a self-cleaving peptide. A “self-cleaving peptide” refers to a peptide that functions such that when the polypeptide comprising the proteins and the self-cleaving peptide is produced, it is immediately “cleaved” or separated into distinct and discrete first and second polypeptides without the need for any external cleavage activity. In some cases, a self-cleaving peptide has a length of from about 15 amino acids to about 40 amino acids. In some cases, a self-cleaving peptide has a length of from about 15 amino acids to about 22 amino acids (e.g., 15, 16, 17, 18, 19, 20, 21, or 22 amino acids). In some cases, a self-cleaving peptide has a length of from about 20 amino acids to about 40 amino acids (e.g., from about 20 amino acids to about 25 amino acids, from about 25 amino acids to about 30 amino acids, from about 30 amino acids to about 35 amino acids, or from about 35 amino acids to about 40 amino acids).

In some cases, a self-cleaving peptide is a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FM DV) and equine rhinitis A virus, the 2A peptide is a short section of about 18 amino acids, which, together with the N-terminal residue of protein 2B (a conserved proline residue) represents an autonomous element capable of mediating “cleavage” at its own C-terminus. Also suitable for use is a “2A-like” peptide.

Suitable “2A-like” self-cleaving peptides include, e.g.:

(SEQ ID NO: 160)
YHADYYKQRLIHDVEMNPGP;
(SEQ ID NO: 161)
HYAGYFADLLIHDIETNPGP;
(SEQ ID NO: 162)
QCTNYALLKLAGDVESNPGP;
(SEQ ID NO: 163)
ATNFSLLKQAGDVEENPGP;
(SEQ ID NO: 164)
AARQMLLLLSGDVETNPGP;
(SEQ ID NO: 165)
RAEGRGSLLTCGDVEENPGP;
(SEQ ID NO: 166)
TRAEIEDELIRAGIESNPGP;
(SEQ ID NO: 167)
TRAEIEDELIRADIESNPGP;
(SEQ ID NO: 168)
AKFQIDKILISGDVELNPGP;
(SEQ ID NO: 169)
SSIIRTKMLVSGDVEENPGP;
(SEQ ID NO: 170)
CDAQRQKLLLSGDIEQNPGP;
(SEQ ID NO: 171)
EGRGSLLTCGDVEENPGP;
and
(SEQ ID NO: 172)
YPIDFGGFLVKADSEFNPGP.

Other suitable self-cleaving peptties sicllude: LLNFDLLKLAGDVESNPGP (SEQ ID

(SEQ ID NO: 173)
LLNFDLLKLAGDVESNPGP;
(SEQ ID NO: 174)
TLNFDLLKLAGDVESNPGP;
(SEQ ID NO: 175)
LLKLAGDVESNPGP;
(SEQ ID NO: 176)
NFDLLKLAGDVESNPGP;
(SEQ ID NO: 177)
QLLNFDLLKLAGDVESNPGP;
(SEQ ID NO: 178)
APVKQTLNFDLLKLAGDVESNPGP;
(SEQ ID NO: 179)
VTELLYRMKRAETYCPRPLLAIHPTEARHKQKIVAPVKQT;
(SEQ ID NO: 180)
LNFDLLKLAGDVESNPGP;
(SEQ ID NO: 181)
LLAIHPTEARHKQKIVAPVKQTLNFDLLKLAGDVESNPGP;
(SEQ ID NO: 182)
EARHKQKIVAPVKQTLNFDLLKLAGDVESNPGP;
and
(SEQ ID NO: 183)
VKQTLNFDLLKLAGDVESNPGP.

### Cleavable Linkers

In some cases, in place of an IRES is a nucleotide sequence encoding a cleavable linker, e.g., a proteolytically cleavable linker.

The proteolytically cleavable linker can include a protease recognition sequence recognized by a protease selected from the group consisting of alanine carboxypeptidase, Armillaria mellea astacin, bacterial leucyl aminopeptidase, cancer procoagulant, cathepsin B, clostripain, cytosol alanyl aminopeptidase, elastase, endoproteinase Arg-C, enterokinase, gastricsin, gelatinase, Gly-X carboxypeptidase, glycyl endopeptidase, human rhinovirus 3C protease, hypodermin C, IgA-specific serine endopeptidase, leucyl aminopeptidase, leucyl endopeptidase, lysC, lysosomal pro-X carboxypeptidase, lysyl aminopeptidase, methionyl aminopeptidase, myxobacter, nardilysin, pancreatic endopeptidase E, picornain 2A, picornain 3C, proendopeptidase, prolyl aminopeptidase, proprotein convertase I, proprotein convertase II, russellysin, saccharopepsin, semenogelase, T-plasminogen activator, thrombin, tissue kallikrein, tobacco etch virus (TEV), togavirin, tryptophanyl aminopeptidase, U-plasminogen activator, V8, venombin A, venombin AB, and Xaa-pro aminopeptidase.

For example, the proteolytically cleavable linker can comprise a matrix metalloproteinase cleavage site, e.g., a cleavage site for a MMP selected from collagenase-1, -2, and -3 (MMP-1, -8, and -13), gelatinase A and B (MMP-2 and -9), stromelysin 1, 2, and 3 (MMP-3, -10, and -11), matrilysin (MMP-7), and membrane metalloproteinases (MT1-MMP and MT2-MMP). For example, the cleavage sequence of MMP-9 is Pro-X-X-Hy (SEQ ID NO:184) (wherein, X represents an arbitrary residue; Hy, a hydrophobic residue), e.g., Pro-X-X-Hy-(Ser/Thr) (SEQ ID NO:185), e.g., Pro-Leu/Gln-Gly-Met-Thr-Ser (SEQ ID NO:186) or Pro-Leu/Gln-Gly-Met-Thr (SEQ ID NO:187). Another example of a protease cleavage site is a plasminogen activator cleavage site, e.g., a uPA or a tissue plasminogen activator (tPA) cleavage site. In some cases, the cleavage site is a furin cleavage site. Specific examples of cleavage sequences of uPA and tPA include sequences comprising Val-Gly-Arg. Another example of a protease cleavage site that can be included in a proteolytically cleavable linker is a tobacco etch virus (TEV) protease cleavage site, e.g., ENLYTQS (SEQ ID NO:188), where the protease cleaves between the glutamine and the serine. Another example of a protease cleavage site that can be included in a proteolytically cleavable linker is an enterokinase cleavage site, e.g., DDDDK (SEQ ID NO:189), where cleavage occurs after the lysine residue. Another example of a protease cleavage site that can be included in a proteolytically cleavable linker is a thrombin cleavage site, e.g., LVPR (SEQ ID NO:190). Additional suitable linkers comprising protease cleavage sites include linkers comprising one or more of the following amino acid sequences: LEVLFQGP (SEQ ID NO:191), cleaved by PreScission protease (a fusion protein comprising human rhinovirus 3C protease and glutathione-S-transferase; Walker et al. (1994) Biotechnol. 12:601); a thrombin cleavage site, e.g., CGLVPAGSGP (SEQ ID NO:192); SLLKSRMVPNFN (SEQ ID NO:193) or SLLIARRMPNFN (SEQ ID NO:194), cleaved by cathepsin B; SKLVQASASGVN (SEQ ID NO:195) or SSYLKASDAPDN (SEQ ID NO:196), cleaved by an Epstein-Barr virus protease; RPKPQQFFGLMN (SEQ ID NO:197) cleaved by MMP-3 (stromelysin); SLRPLALWRSFN (SEQ ID NO:198) cleaved by MMP-7 (matrilysin); SPQGIAGQRNFN (SEQ ID NO:199) cleaved by MMP-9; DVDERDVRGFASFL SEQ ID NO:200) cleaved by a thermolysin-like MMP; SLPLGLWAPNFN (SEQ ID NO:201) cleaved by matrix metalloproteinase 2(MMP-2); SLLIFRSWANFN (SEQ ID NO:202) cleaved by cathespin L; SGVVIATVIVIT (SEQ ID NO:203) cleaved by cathepsin D; SLGPQGIWGQFN (SEQ ID NO:204) cleaved by matrix metalloproteinase 1 (MMP-1); KKSPGRVVGGSV (SEQ ID NO:205) cleaved by urokinase-type plasminogen activator; PQGLLGAPGILG (SEQ ID NO:206) cleaved by membrane type 1 matrixmetalloproteinase (MT-MMP); HGPEGLRVGFYESDVMGRGHARLVHVEEPHT (SEQ ID NO:207) cleaved by stromelysin 3 (or MMP-11), thermolysin, fibroblast collagenase and stromelysin-1; GPQGLAGQRGIV (SEQ ID NO:208) cleaved by matrix metalloproteinase 13 (collagenase-3); GGSGQRGRKALE (SEQ ID NO:209) cleaved by tissue-type plasminogen activator(tPA); SLSALLSSDIFN (SEQ ID NO:210) cleaved by human prostate-specific antigen; SLPRFKIIGGFN (SEQ ID NO:211) cleaved by kallikrein (hK3); SLLGIAVPGNFN (SEQ ID NO:212) cleaved by neutrophil elastase; and FFKNIVTPRTPP (SEQ ID NO:213) cleaved by calpain (calcium activated neutral protease).

### Signal Sequences

An mRNA of the present disclosure can include a nucleotide sequence encoding a peptide that facilitates trafficking of an operably linked polypeptide. As used herein, a “signal sequence” or “signal peptide” is a polynucleotide or polypeptide, respectively, which is from about 9 to 200 nucleotides in length (encoding a polypeptide of from about 3 amino acids to about 60 amino acids in length; e.g., from about 15 amino acids to about 25 amino acids in length), which is incorporated at the 5′ (or N-terminus) of the coding region or polypeptide encoded, respectively. In some cases, addition of these sequences results in trafficking of the encoded polypeptide to the endoplasmic reticulum through one or more secretory pathways. In some cases, addition of these sequences results in secretion of the encoded polypeptide from the cell. Some signal peptides are cleaved from the protein by signal peptidase after the proteins are transported.

Many signal sequences are known in the art; any known signal sequence can be used herein. The following are non-limiting examples of suitable signal sequences.
ATGGACGCAATGAAACGGGGTCTGTGCTGCGTACTCTTGCTTTGTGGAGCTG TGTTCGTGTCACCTTCT (SEQ ID NO:214) (encoding tissue-type plasminogen activator (t-PA) signal peptide MDAMKRGLCCVLLLCGAVFVSPS (SEQ ID NO:63);
ATGATGCCATCCTCAGTC TCATGGGGTATTTTGCTC TTGGCGGGTCTGTGCTGT CTCGTGCCGGTGTCGCTC GCA (SEQ ID NO:215) (encoding α-1-anti-trypsin signal peptide MMPSSVS WGILLAGL CCLVPVSLA (SEQ ID NO:216); and
ATGGCGACGCCGCTGCCT CCGCCCTCCCCGCGGCAC CTGCGGCTGCTGCGGCTG CTGCTCTCCGCCCTCGTC CTCGGC (SEQ ID NO:217) (encoding: MATPLPPP SPRHLRLL RLLLSG (SEQ ID NO:218).

### HCV E1/E2 Heterodimers; HCV E2 Polypeptides; HCV E1 Polypeptides

An RNA molecule of the present disclosure can comprise: i) a nucleotide sequence encoding an HCV E1 polypeptide, and not an HCV E2 polypeptide; or ii) a nucleotide sequence encoding an HCV E2 polypeptide, and not an HCV E1 polypeptide; or iii) a first nucleotide sequence encoding an HCV E1 polypeptide; and a second nucleotide sequence encoding an HCV E2 polypeptide; or iv) a nucleotide sequence encoding an HCV E1 polypeptide and an HCV E2 polypeptide.

HCV E1, E2, and E1/E2 polypeptides that can be encoded by an RNA molecule of the present disclosure include wild-type HCV E1 polypeptides, wild-type HCV E2 polypeptides and wild-type HCV E1/E2 polypeptides, i.e., polypeptides comprising wild-type amino acid sequences found in natural isolates. HCV E2 polypeptides that can be encoded by an RNA molecule of the present disclosure include wild-type E2 polypeptides and variant E2 polypeptides. HCV E1 polypeptides that can be encoded by an RNA molecule of the present disclosure include wild-type E1 polypeptides and variant E1 polypeptides.

### E2 Polypeptides

An E2 polypeptide that is encoded by an RNA molecule of the present disclosure can have a length of from about 200 amino acids (aa) to about 250 aa, from about 250 aa to about 275 aa, from about 275 aa to about 300 aa, from about 300 aa to about 325 aa, from about 325 aa to about 350 aa, or from about 350 aa to about 365 aa. In some cases, an HCV E2 polypeptide that is encoded by an RNA molecule of the present disclosure is an HCV E2 ectodomain polypeptide (e.g., lacking the transmembrane domain of a naturally-occurring E2 polypeptide). In some cases, an HCV E2 polypeptide that is encoded by an RNA molecule of the present disclosure is a full-length HCV E2 polypeptide. In some cases, an HCV E2 polypeptide that is encoded by an RNA molecule of the present disclosure is a soluble HCV E2 polypeptide.

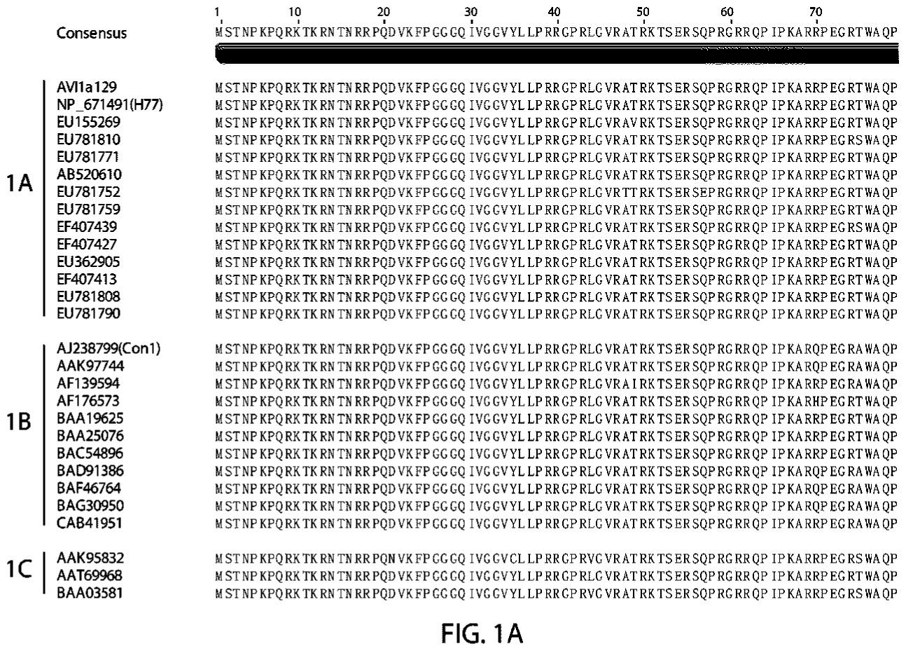

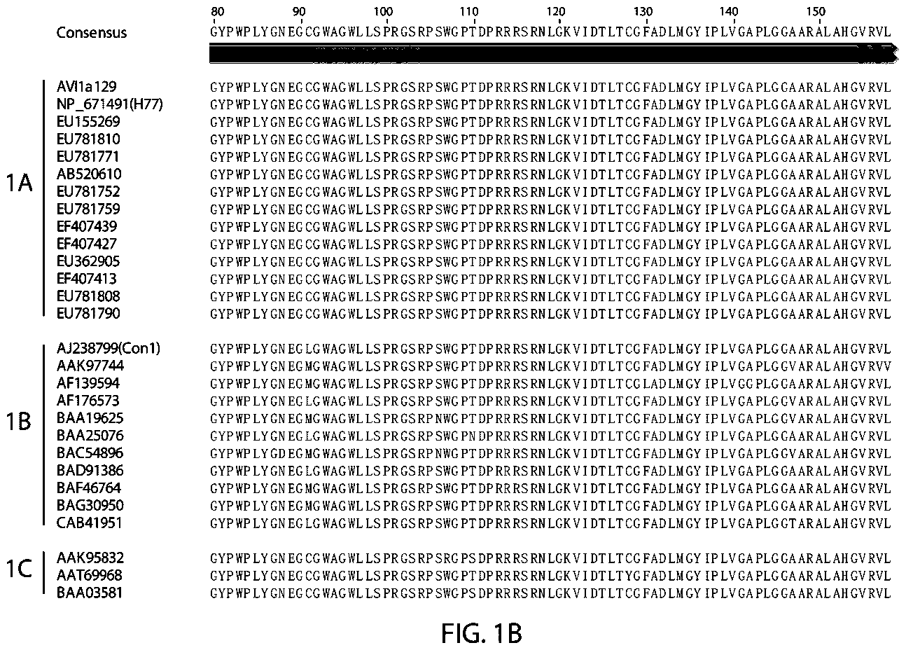

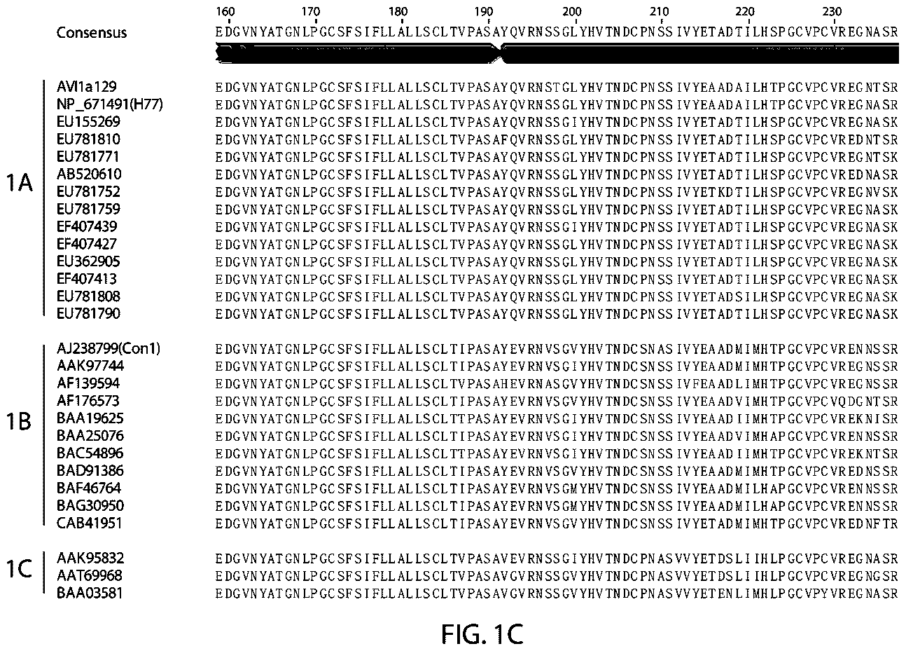

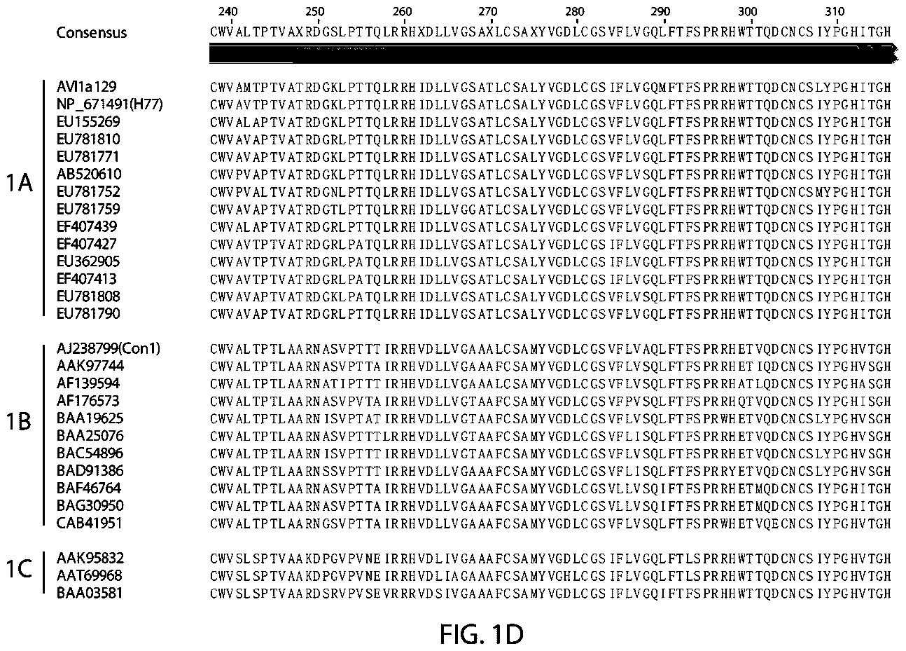

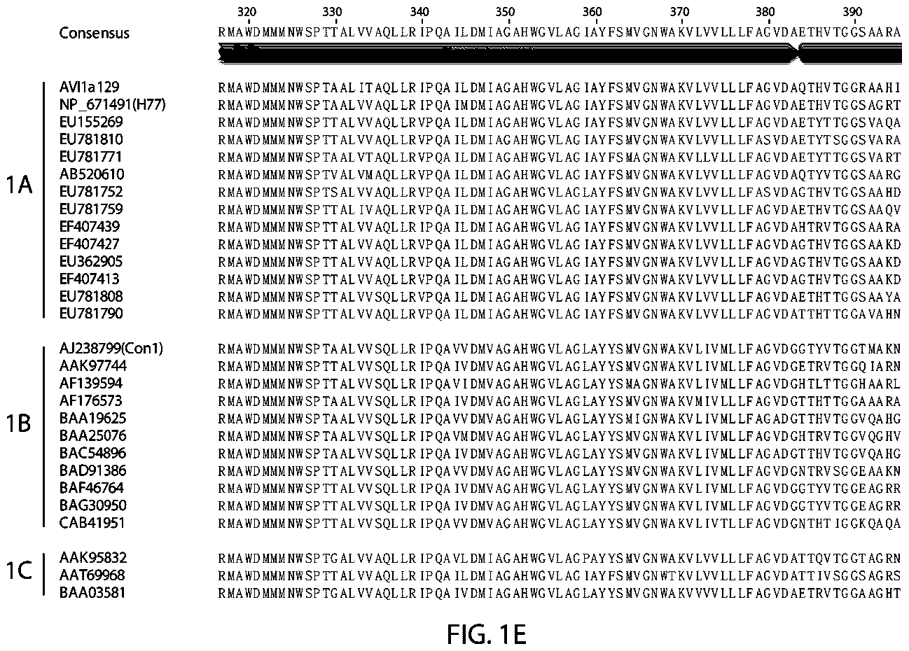

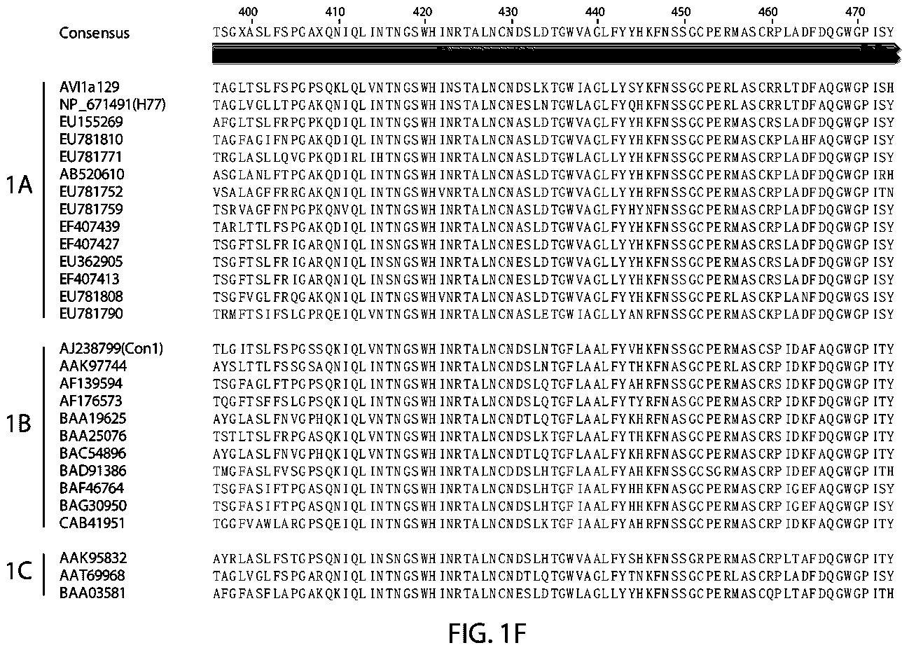

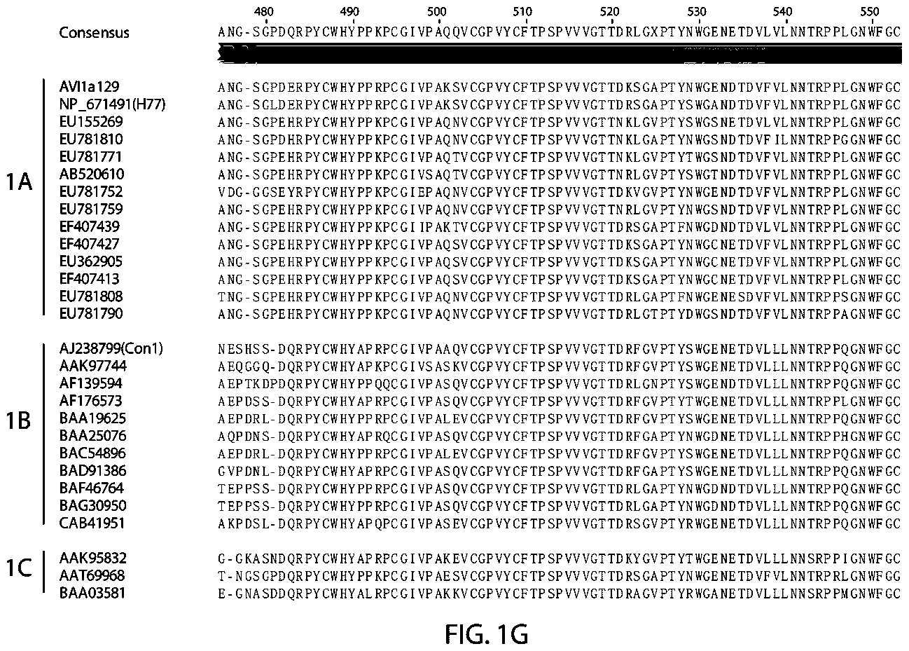

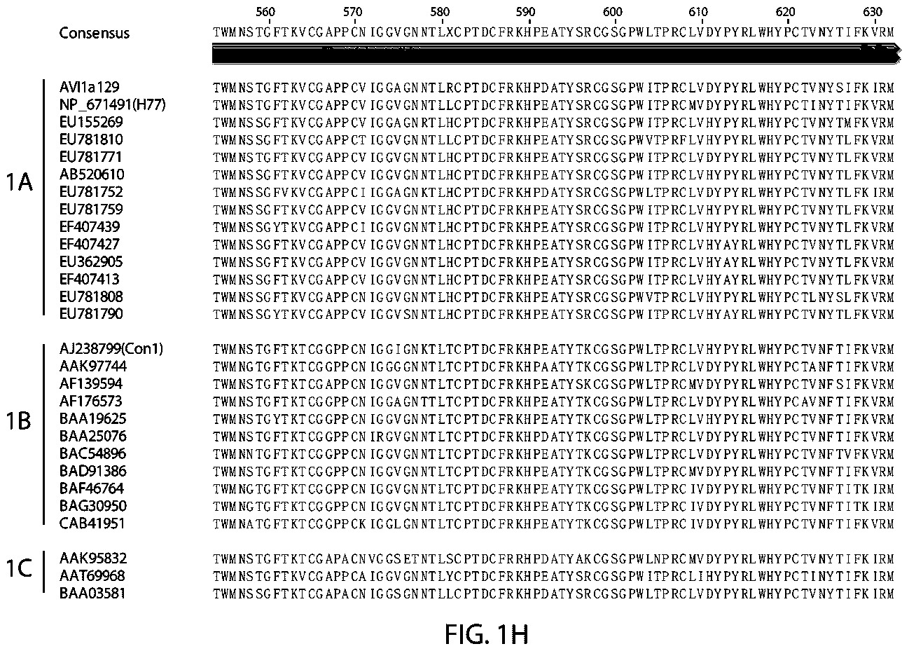

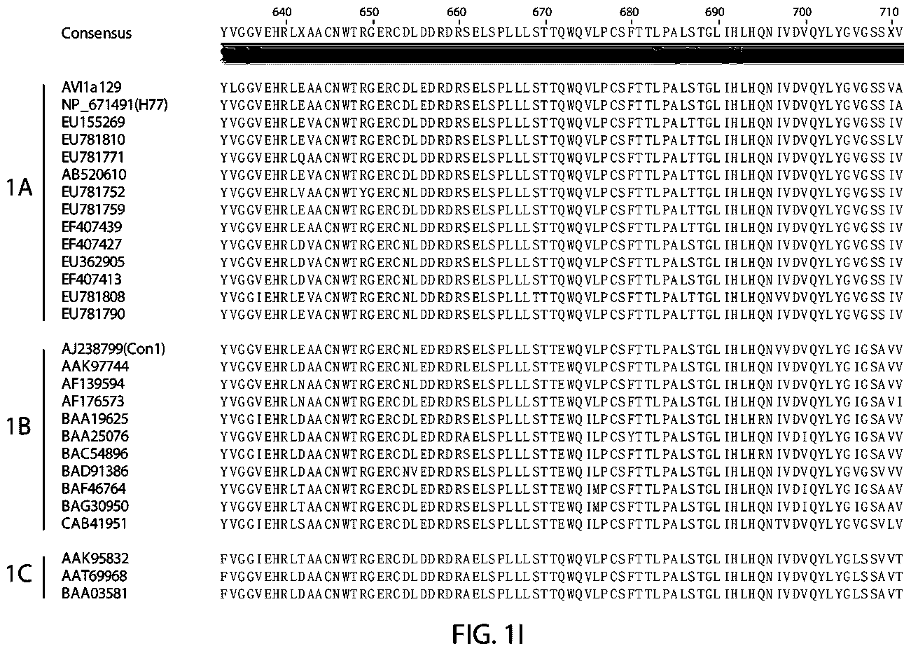

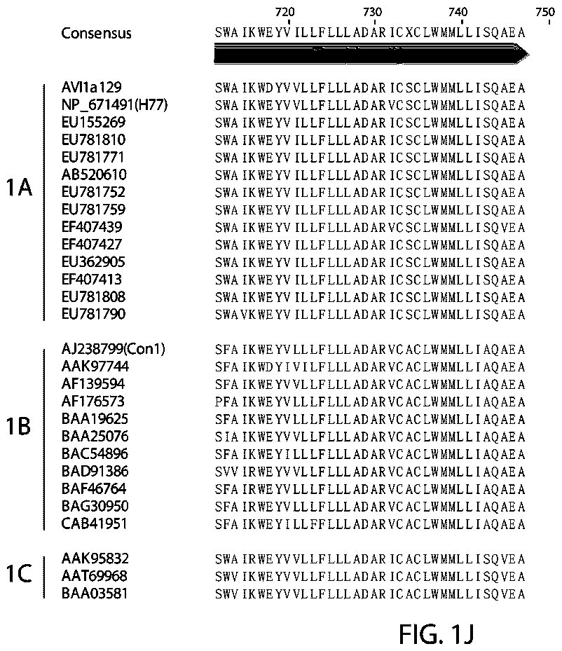

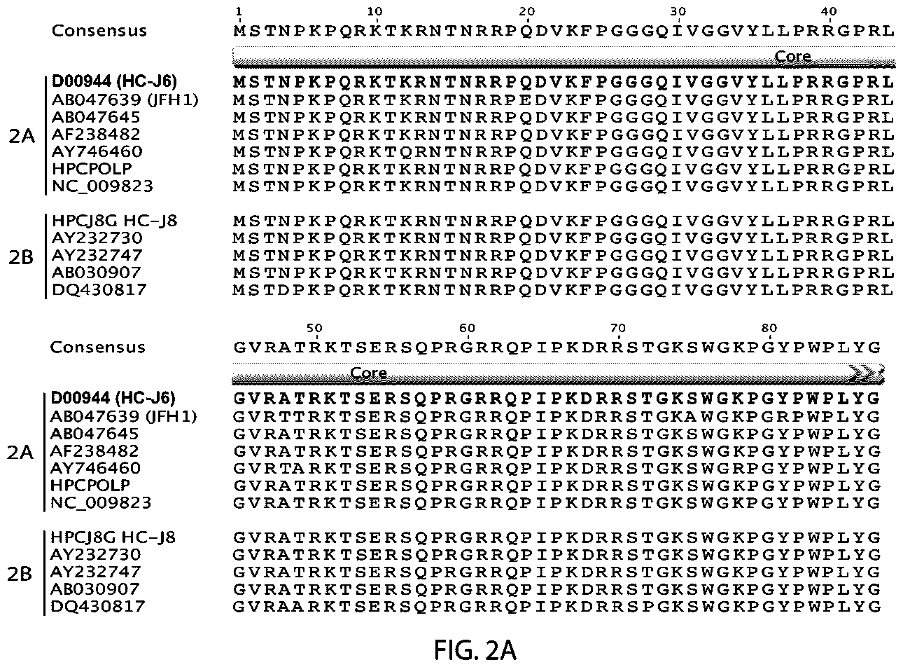

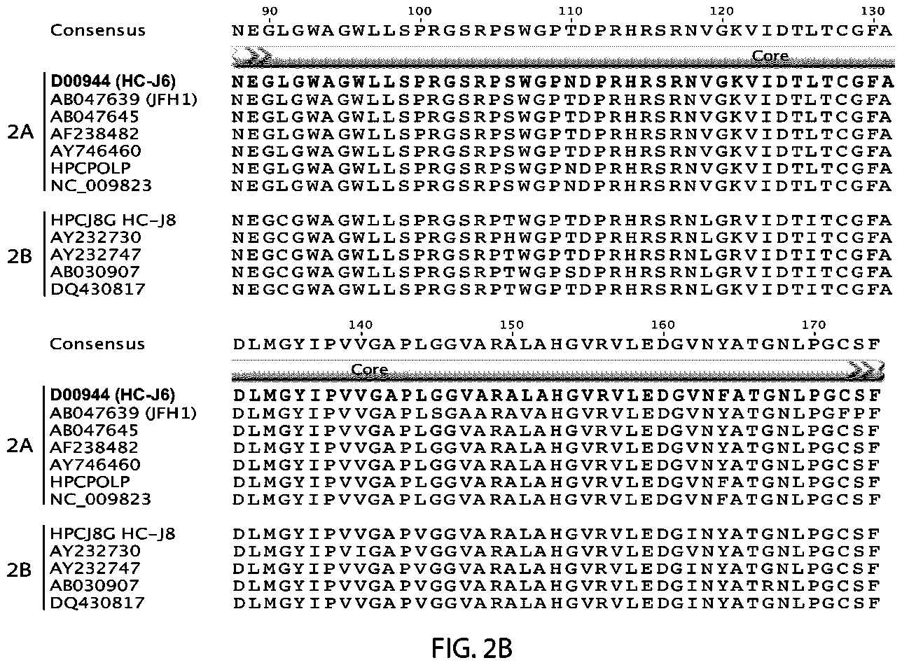

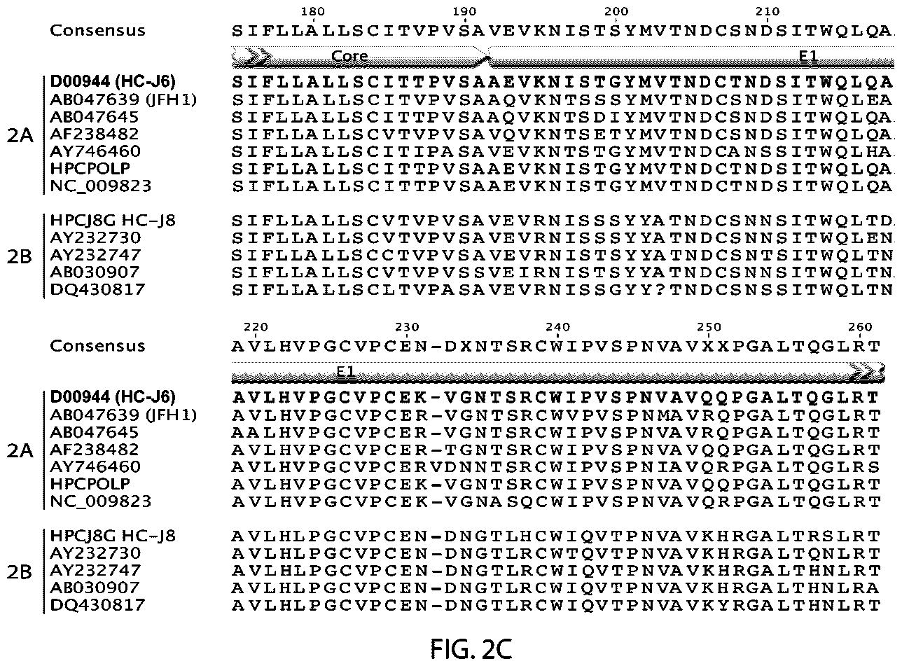

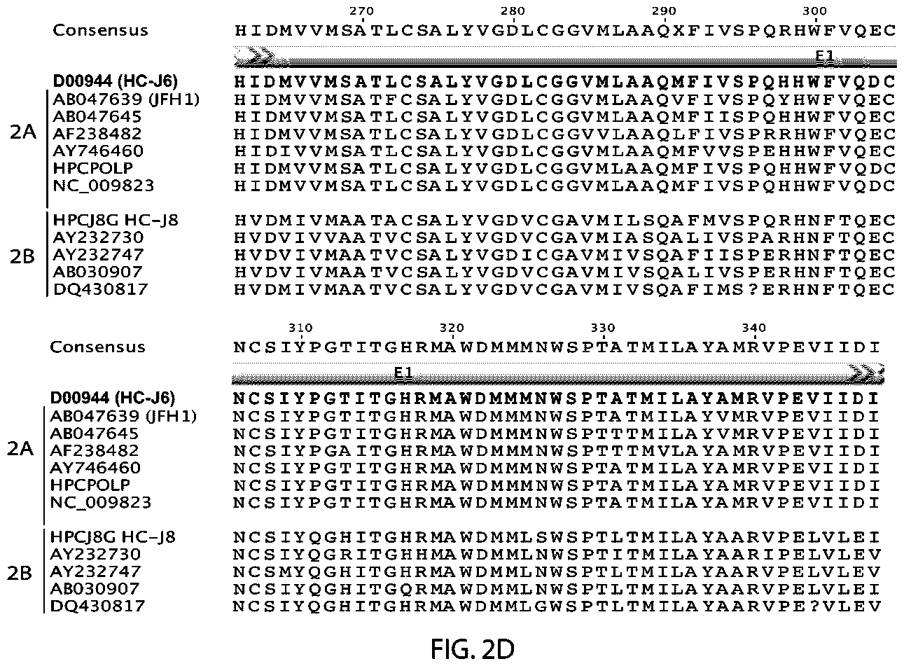

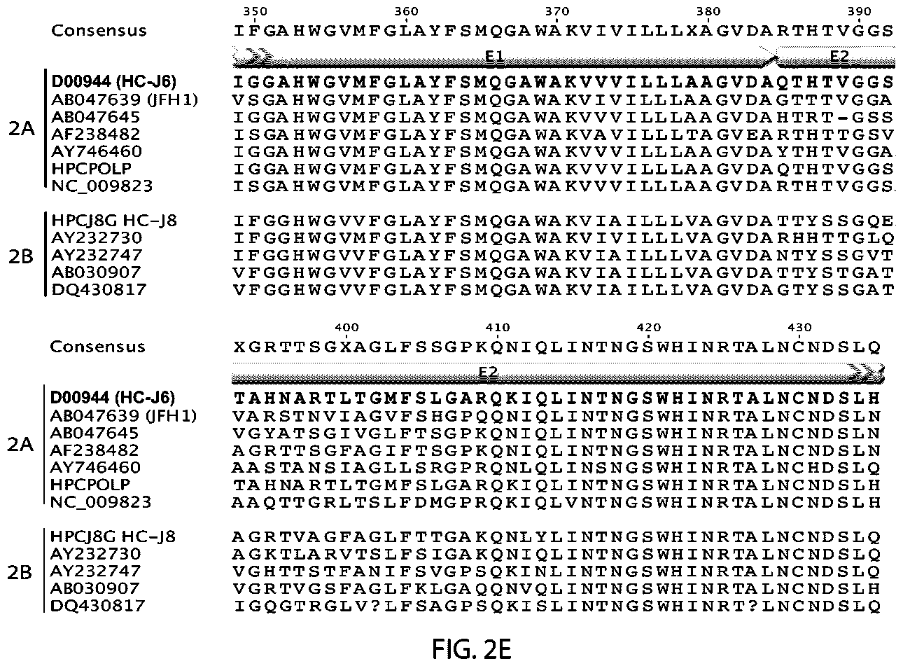

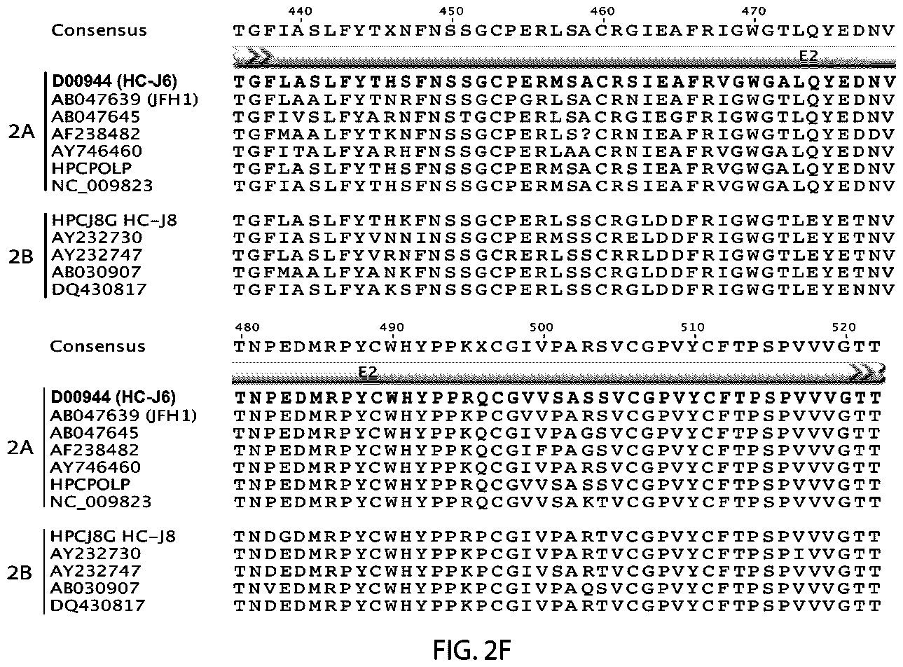

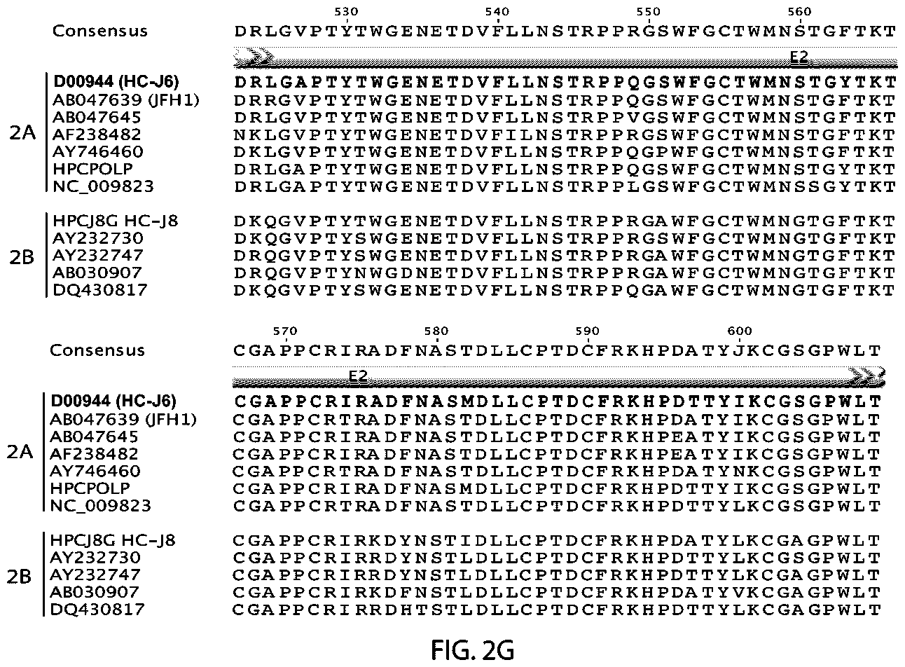

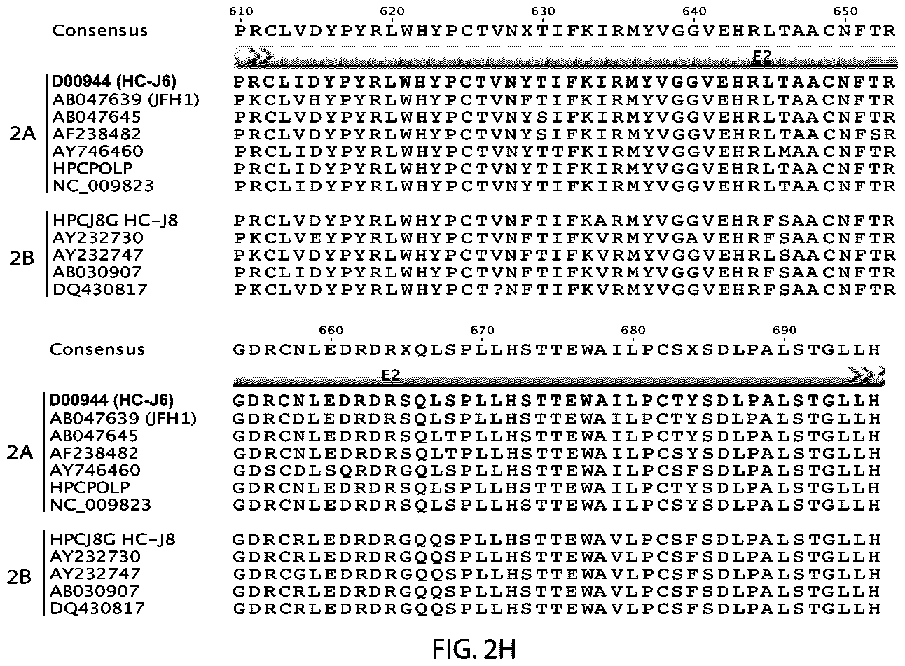

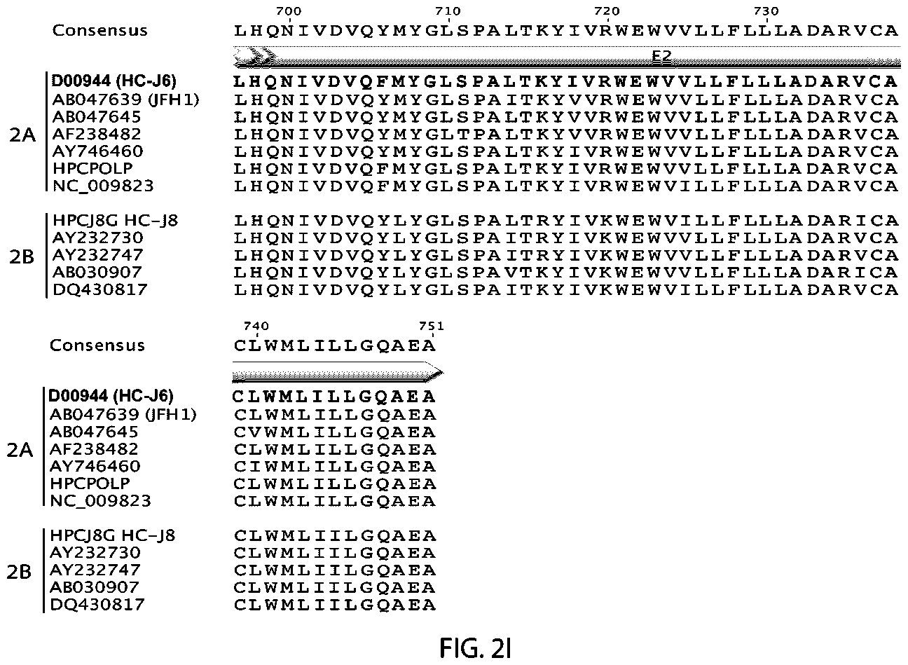

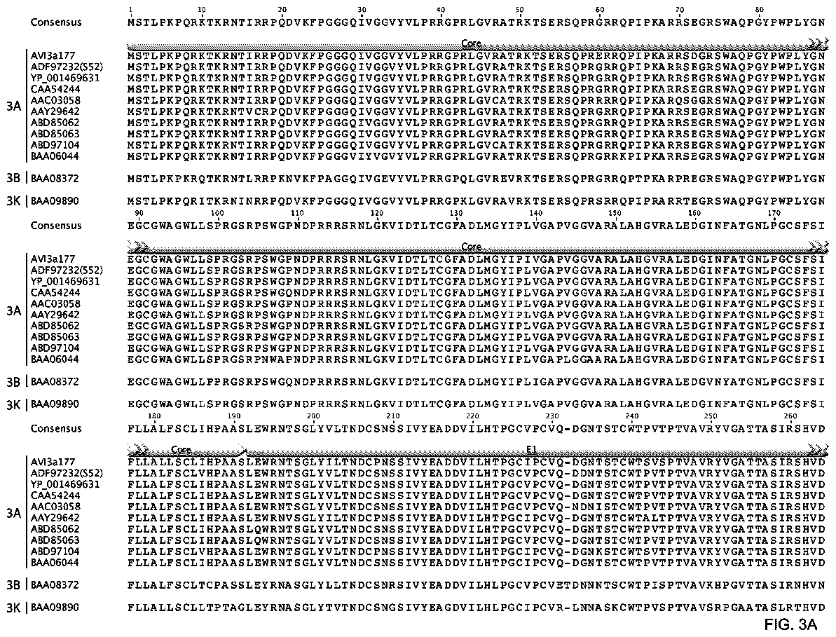

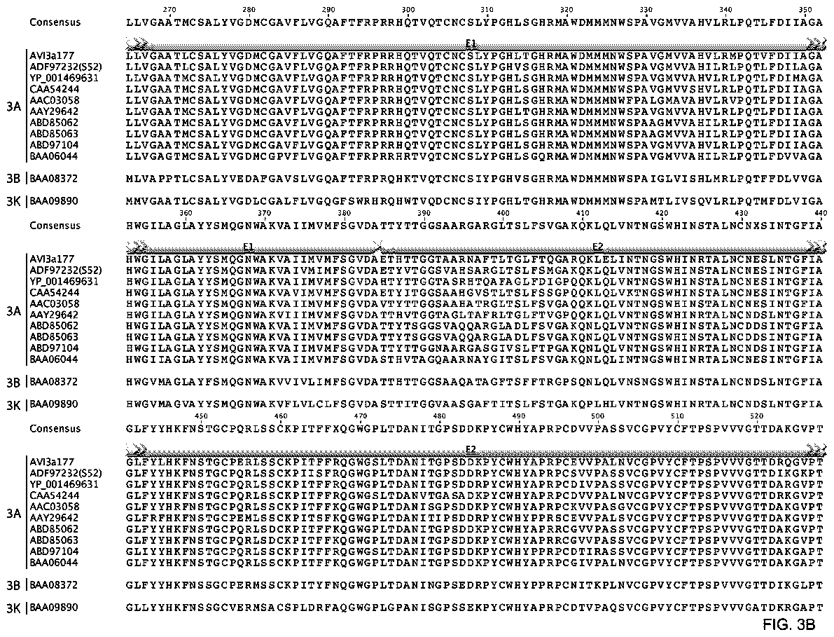

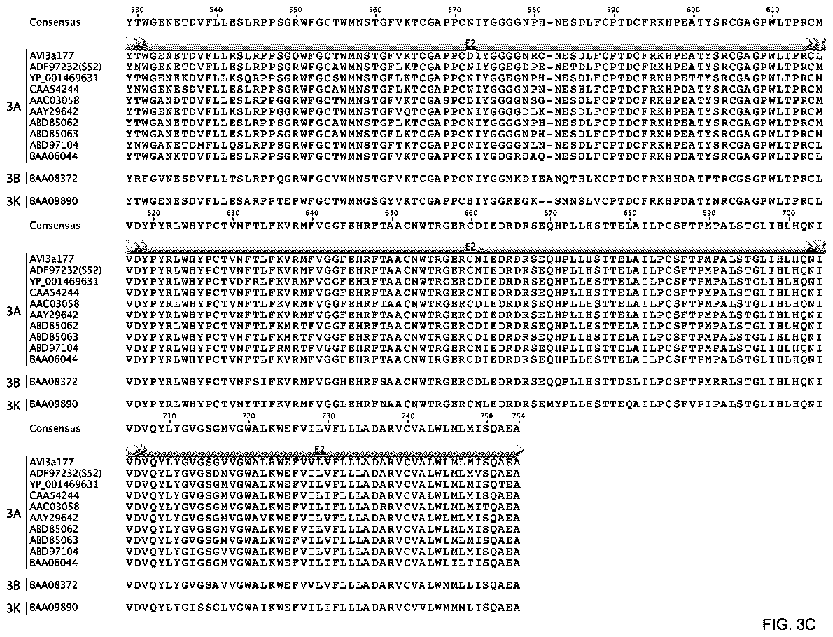

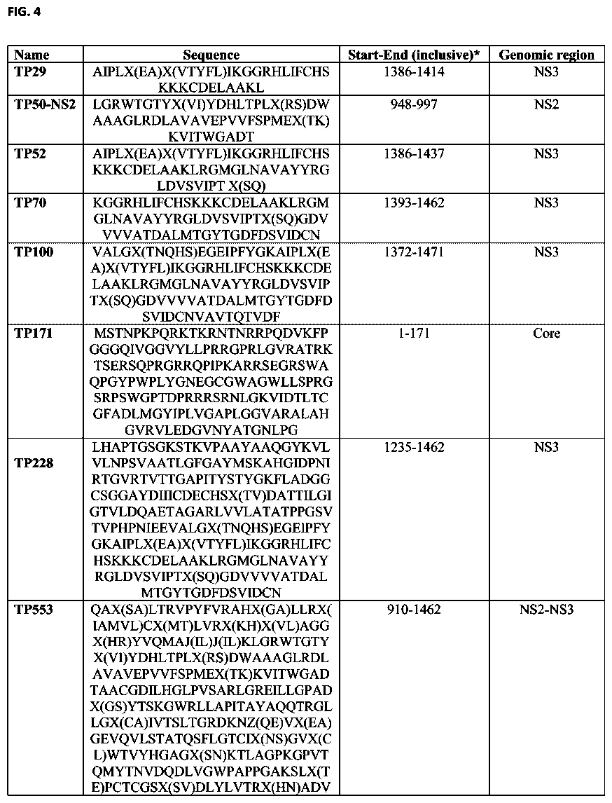

In FIG. 1A-AC, the amino acid sequence of E2 is amino acid 384 to amino acid 746. In FIG. 2A-2B, the amino acid sequence of E2 is amino acid 384 to amino acid 751. In FIG. 3A-3C, the amino acid sequence of E2 is amino acid 385 to amino acid 754. In FIG. 20A-20B, the amino acid sequence of E2 is amino acid 384 to amino acid 750. As used herein, an “E2 polypeptide” includes a precursor E2 protein, including the signal sequence; includes a mature E2 polypeptide which lacks this sequence; and includes an E2 polypeptide with a heterologous signal sequence. An E2 polypeptide can include a C-terminal membrane anchor sequence which occurs at approximately amino acid positions 715-730 and may extend as far as approximately amino acid residue 746 (see, Lin et al., J. Virol. (1994) 68:5063-5073).

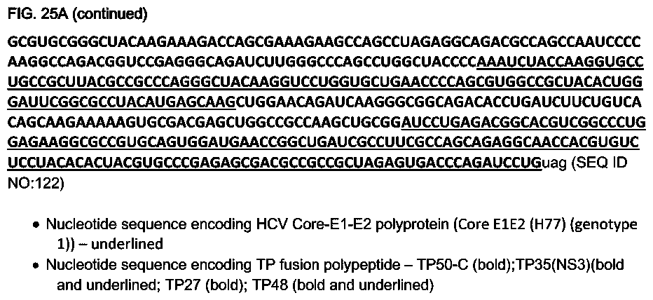

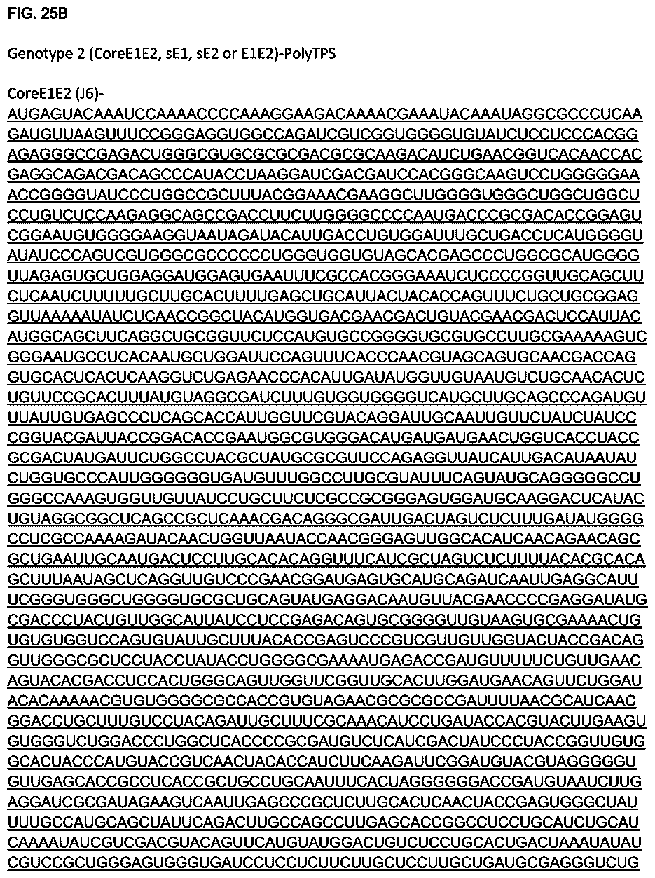

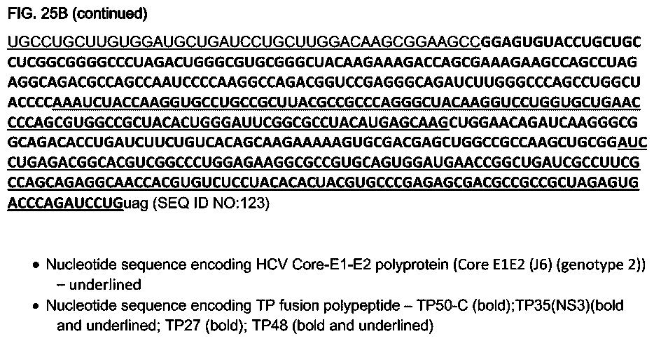

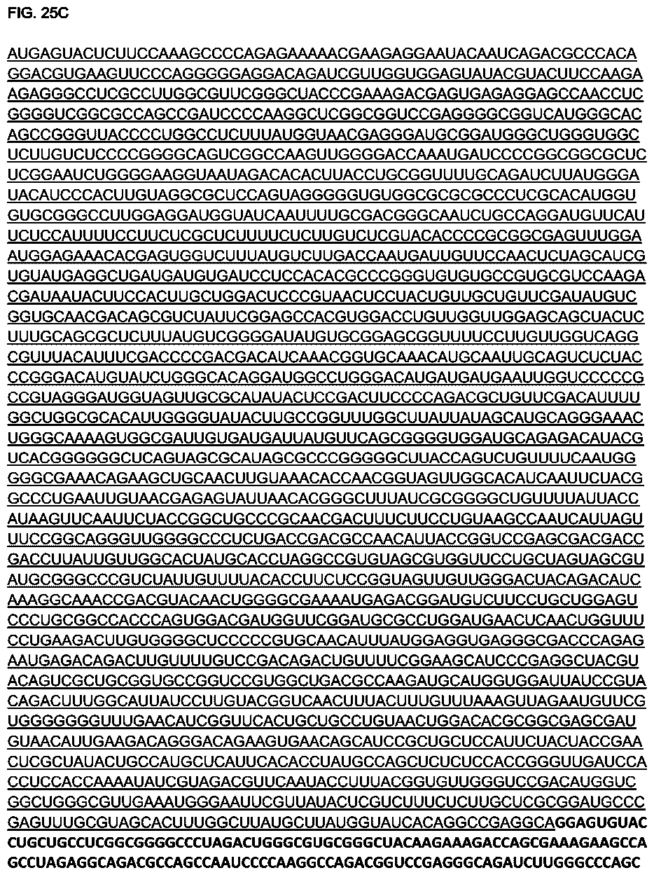

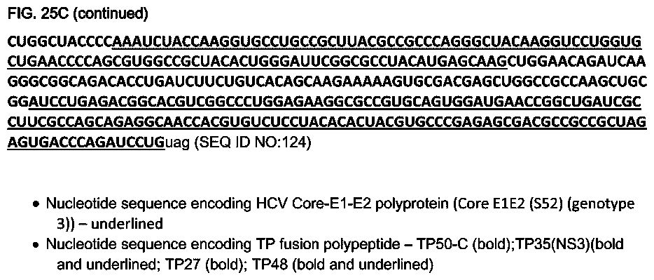

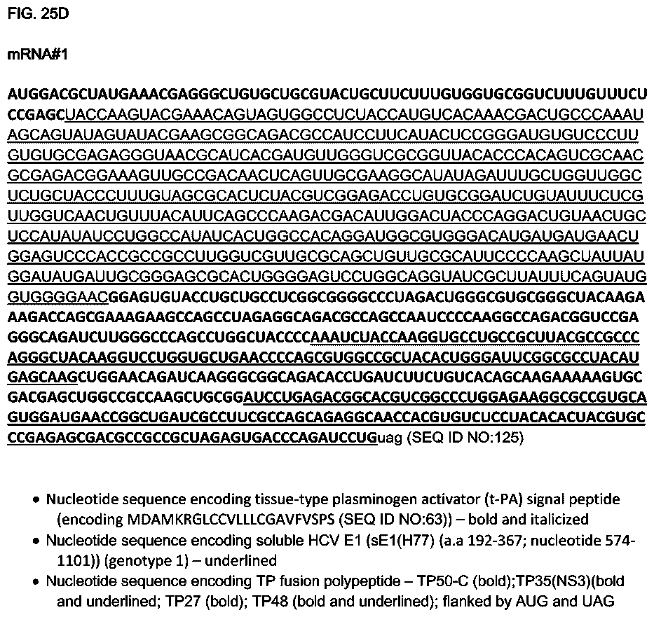

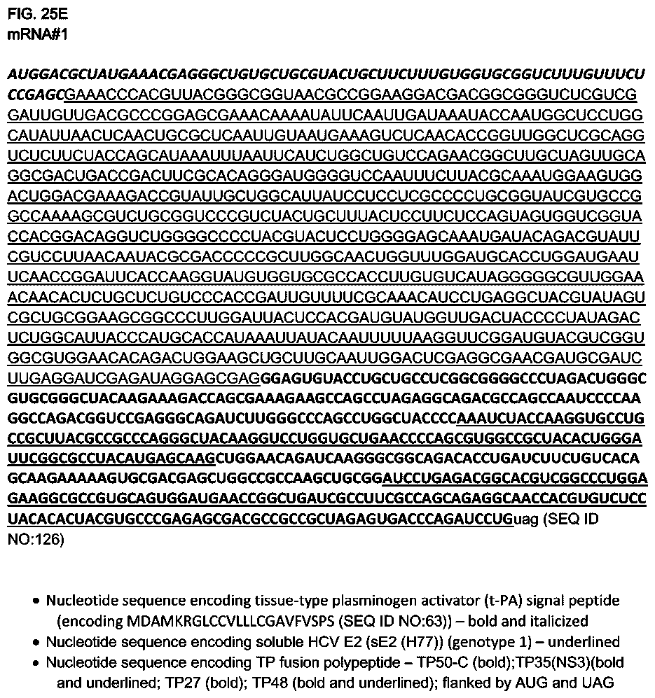

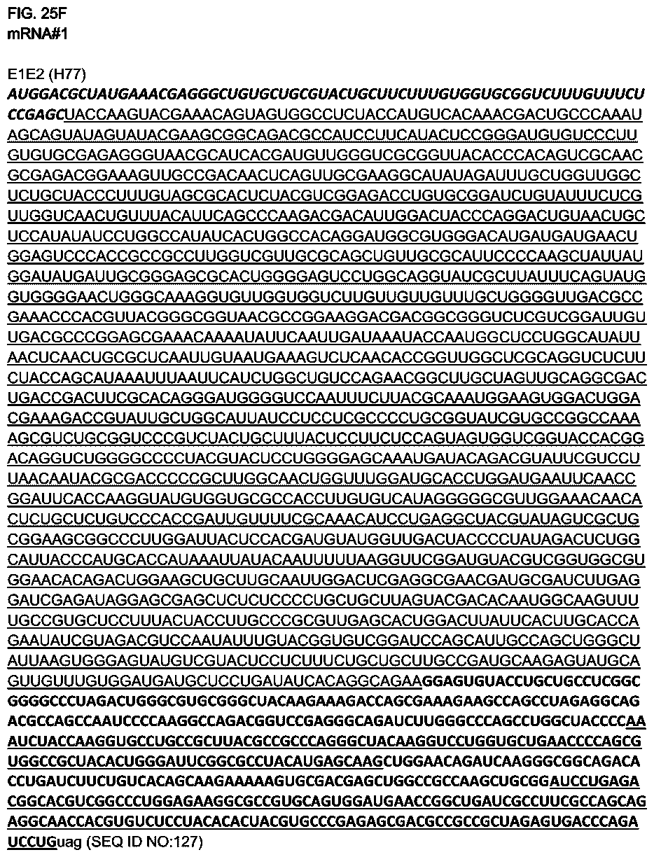

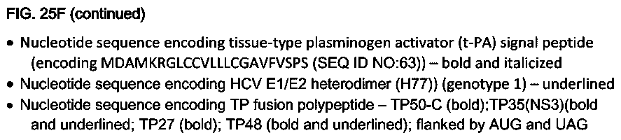

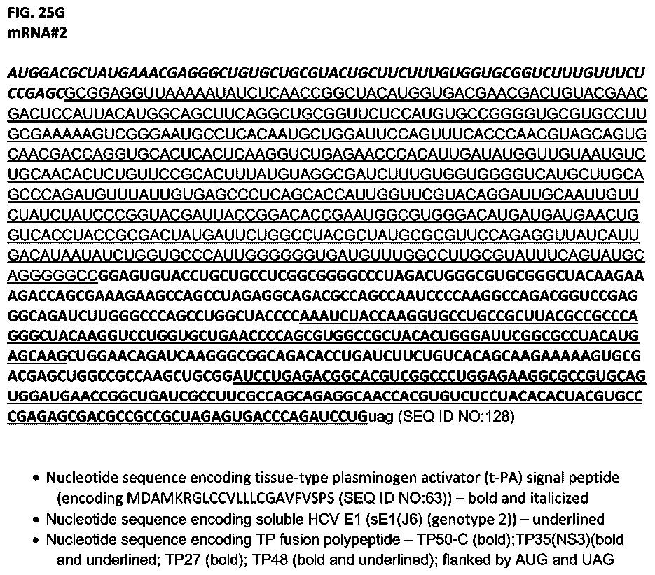

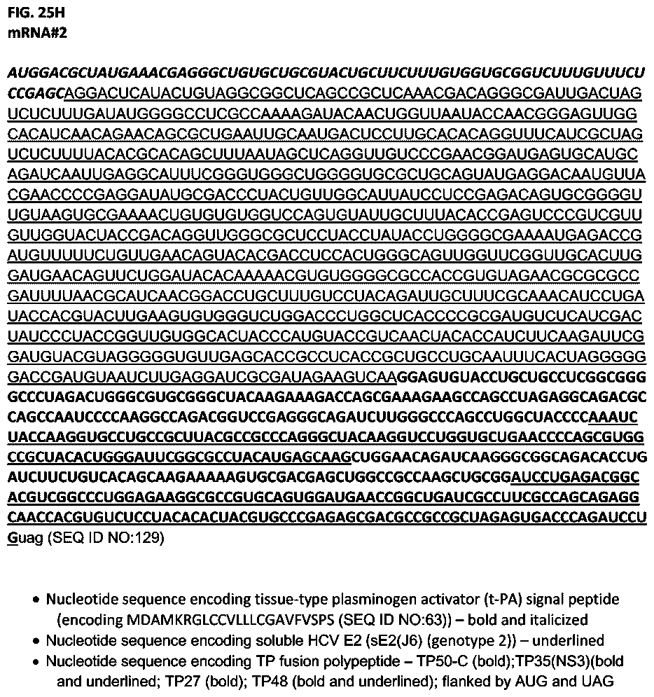

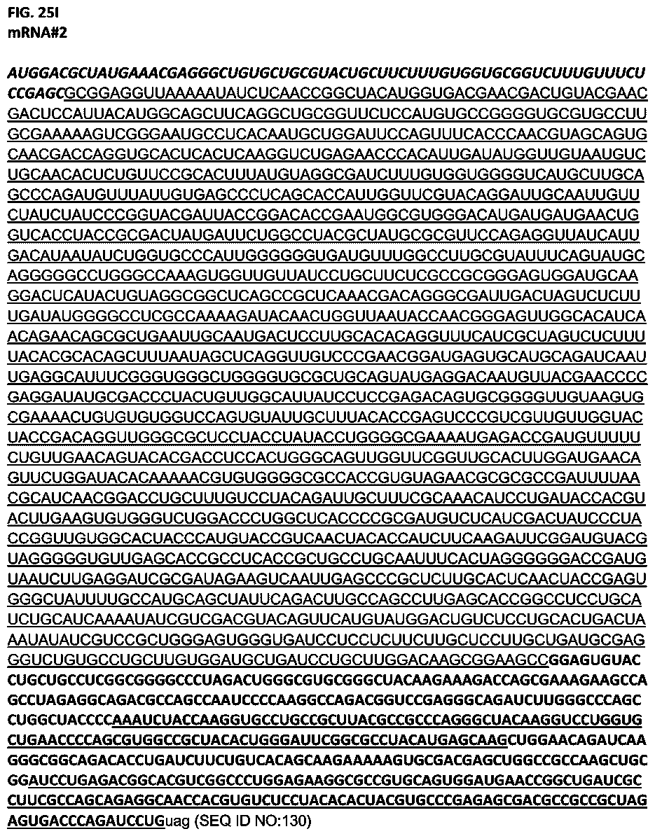

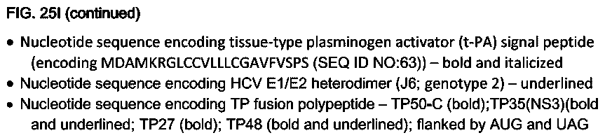

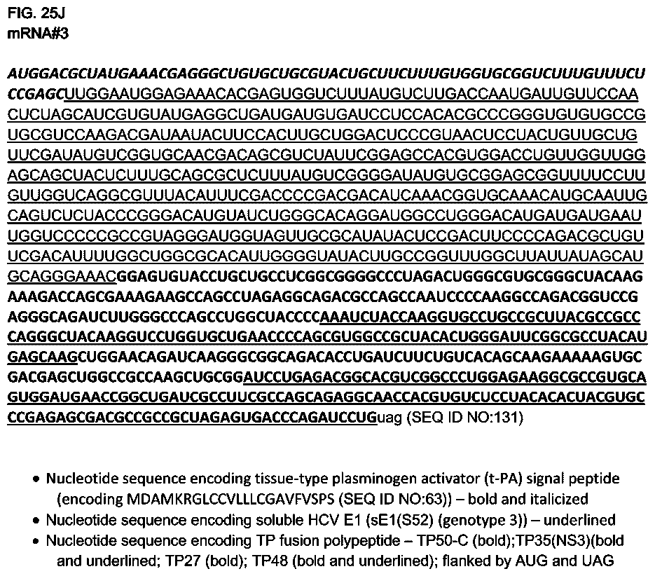

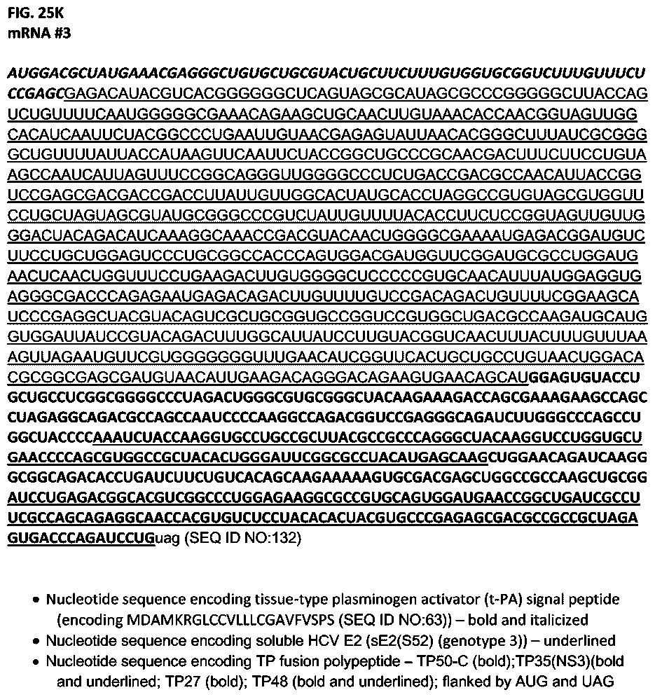

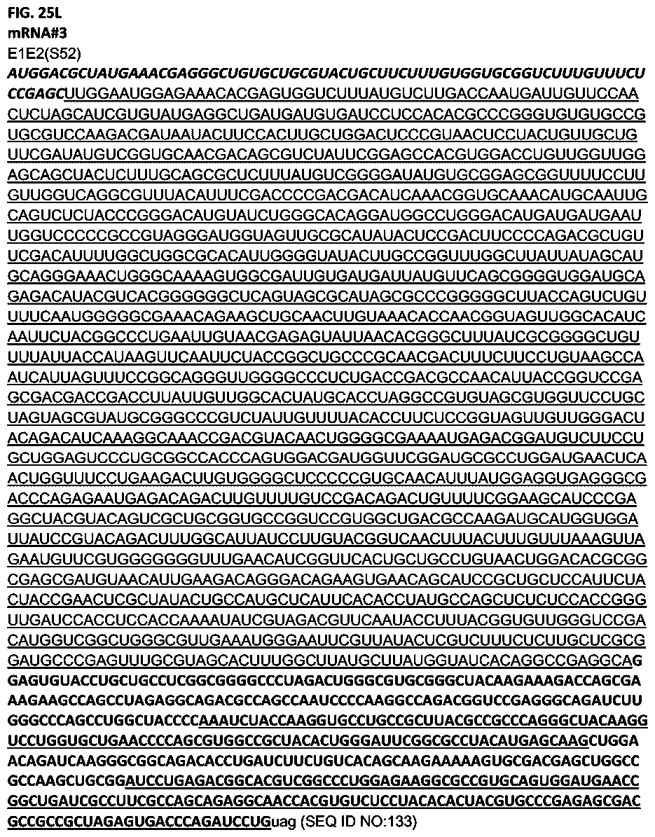

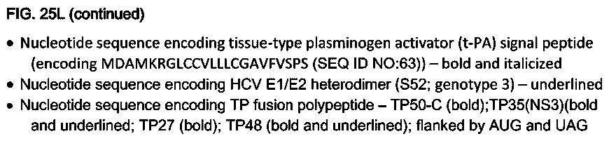

In some cases, a E2 polypeptide that is encoded by an RNA molecule of the present disclosure lacks a portion of its C-terminal region, e.g., from about amino acid 715 to the C-terminus; from about amino acid 625 to the C-terminus; from about amino acid 661 to the C-terminus; from about amino acid 655 to the C-terminus; from about amino acid 500 to the C-terminus, where the amino acid numbering is with reference to the numbering in FIG. 1A-IC. See, e.g., U.S. Pat. No. 6,521,423. An E2 polypeptide that lacks a C-terminal region, e.g., a C-terminal membrane anchor sequence, is also referred to herein as a “soluble” E2 polypeptide. In some cases, a soluble E2 polypeptide comprises amino acids 384-717 of any one of the amino acid sequences depicted in FIG. 1A-IC, FIG. 2A-2B, FIG. 3A-3C, or FIG. 20A-20B. In some cases, a soluble E2 polypeptide comprises amino acids 384-661 of any one of the amino acid sequences depicted in FIG. 1A-IC, FIG. 2A-2B, FIG. 3A-3C, or FIG. 20A-20B; and lacks the C-terminal portion, e.g., from amino acids 662-746, from amino acids 662 to 751, or from amino acids 662 to 754, or from amino acids 662 to 750. A non-limiting example of a nucleotide sequence encoding a soluble E2 polypeptide is provided in FIG. 25H. See, e.g., Guest et al. (2021) Proc. Natl. Acad. Sci. USA 18:e2015149118; Cao et al. (2019) PLoS Pathogens 15:e1007759.

An E2 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 1A-1C, FIG. 2A-2C, FIG. 3A-3C, or FIG. 20A-20B. An E2 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, or at least about 75%, amino acid sequence identity to an amino acid sequence of an E2 polypeptide depicted in FIG. 1A-1C, FIG. 2A-2C, FIG. 3A-3C, or FIG. 20A-20B.

An E2 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 1A-1C. For example, an E2 polypeptide of genotype 1 that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence depicted in FIG. 1A-1C. For example, an E2 polypeptide of genotype 1A that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence identified as 1A and depicted in FIG. 1A-1C. For example, an E2 polypeptide of genotype 1B that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence identified as 1B and depicted in FIG. 1A-IC. For example, an E2 polypeptide of genotype 1C that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-746 of an amino acid sequence identified as 1C and depicted in FIG. 1A-IC.

An E2 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 2A-2C. For example, an E2 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-751 of an amino acid sequence depicted in FIG. 2A-2C. For example, an E2 polypeptide of genotype 2A that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-751 of the “consensus” amino acid sequence depicted in FIG. 2A-2C. For example, an E2 polypeptide of genotype 2B that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-751 of the “consensus” amino acid sequence depicted in FIG. 2A-2C.

An E2 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E2 polypeptide depicted in FIG. 3A-3C. For example, an E2 polypeptide of genotype 3 that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of an amino acid sequence depicted in FIG. 3A-3C. For example, an E2 polypeptide of genotype 3A that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of an amino acid sequence identified as 3A and depicted in FIG. 3A-3C. For example, an E2 polypeptide of genotype 3B that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of the amino acid sequence identified as 3B and depicted in FIG. 3A-3C. For example, an E2 polypeptide of genotype 3K that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 385-754 of the amino acid sequence identified as 3K and depicted in FIG. 3A-3C.

An E2 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the amino acid sequence of the E2 polypeptide depicted in FIG. 20A-20B. For example, an E2 polypeptide of genotype 7A that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 384-750 of the amino acid sequence depicted in FIG. 20A-20B.

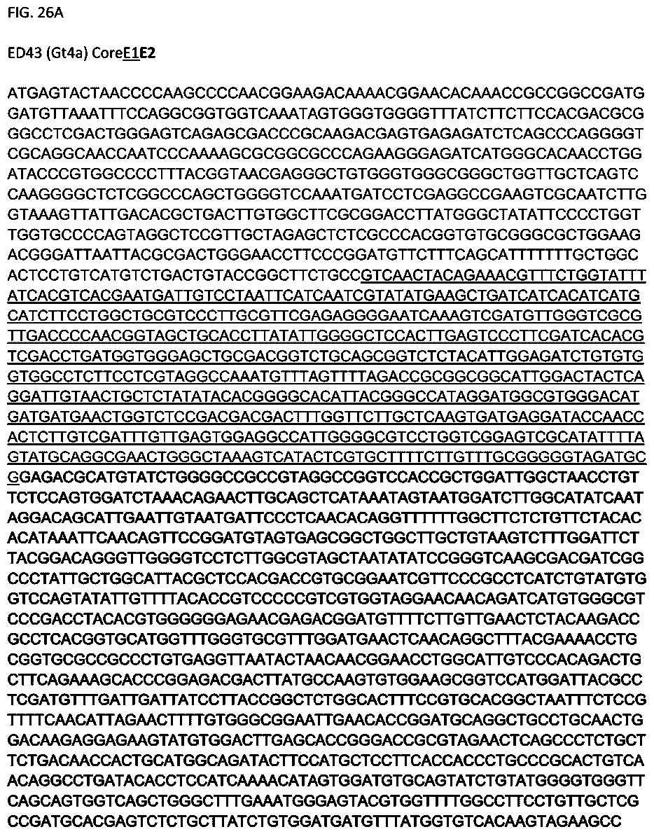

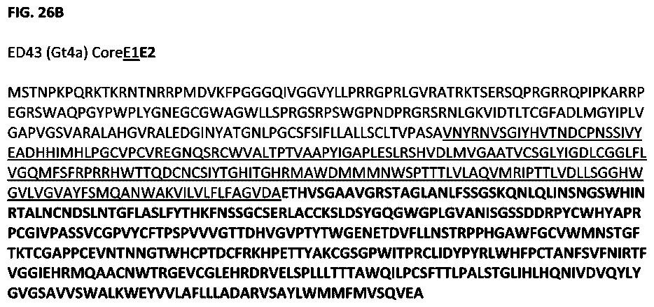

An E2 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the amino acid sequence of a genotype 4 E2 polypeptide depicted in FIG. 26B, where the E2 polypeptide amino acid sequence is in bold in FIG. 26B. An example of a nucleotide sequence encoding an E2 polypeptide of genotype 4 is provided in FIG. 26A, where the nucleotide sequence encoding the E2 polypeptide is in bold.

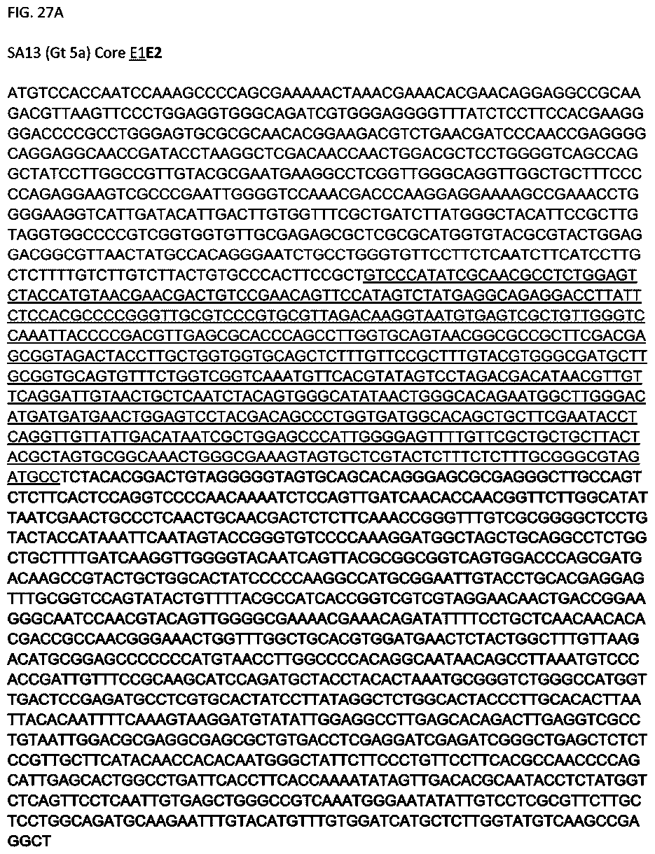

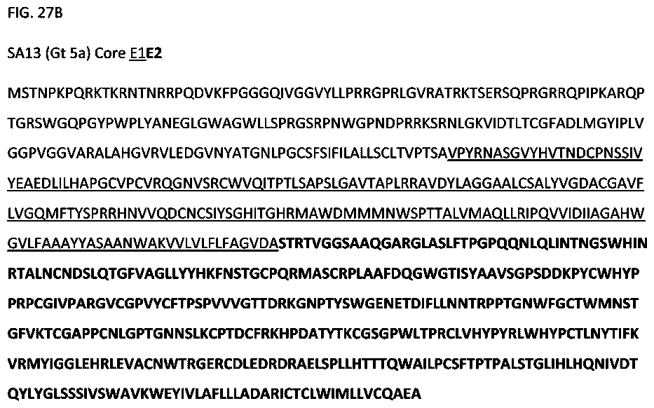

An E2 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the amino acid sequence of a genotype 5 E2 polypeptide depicted in FIG. 27B, where the E2 polypeptide amino acid sequence is in bold in FIG. 27B. An example of a nucleotide sequence encoding an E2 polypeptide of genotype 5 is provided in FIG. 27A, where the nucleotide sequence encoding the E2 polypeptide is in bold.

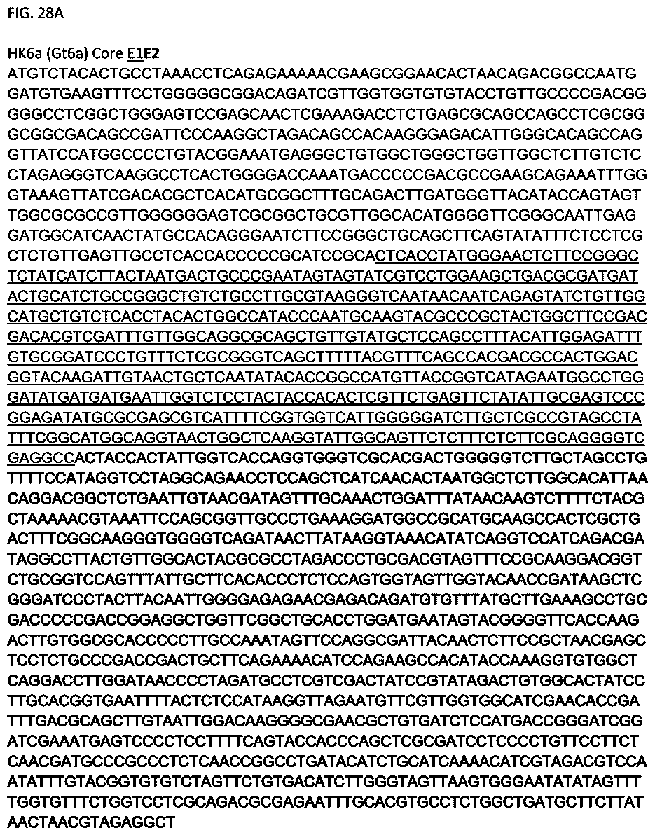

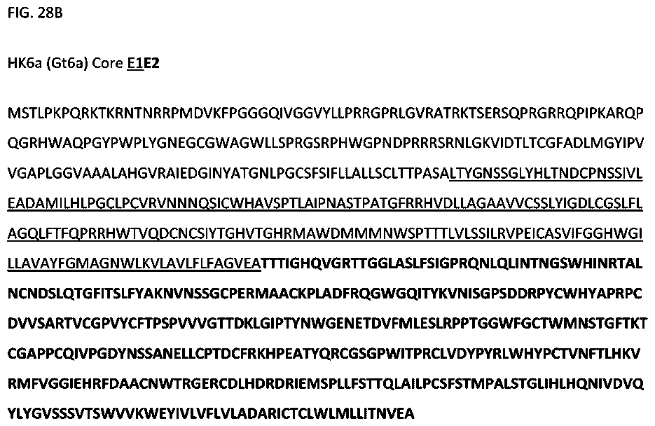

An E2 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the amino acid sequence of a genotype 6 E2 polypeptide depicted in FIG. 28B, where the E2 polypeptide amino acid sequence in bold in FIG. 28B. An example of a nucleotide sequence encoding an E2 polypeptide of genotype 6 is provided in FIG. 28A, where the nucleotide sequence encoding the E2 polypeptide is in bold.

### Genotype 1—H77

In some cases, an E2 polypeptide that is encoded by an RNA molecule of the present disclosure is a genotype 1 E2 polypeptide and comprises an amino acid sequence having at least about 95%, at least about 98%, at least about 99%, or 100%, amino acid sequence identity to amino acids 384 to 746 of the amino acid sequence identified in FIG. 1A-IC as “NP_671491 (H77)” (also referred to herein as “H77”); and can have a length of about 363 amino acids (e.g., from 360 amino acids to 365 amino acids); where such a polypeptide is referred to as an “H77 E2 polypeptide.” In some cases, an E2 polypeptide that is encoded by an RNA molecule of the present disclosure comprises an amino acid sequence having at least about 95%, at least about 98%, at least about 99%, or 100%, amino acid sequence identity to amino acids 384 to 661 of the H77 amino acid sequence depicted in FIG. 1A-IC; and can have a length of about 278 amino acids (e.g., from about 275 amino acids to about 280 amino acids), where such an E2 polypeptide is referred to as a “soluble” H77 E2 polypeptide. In some cases, an E2 polypeptide that is encoded by an RNA molecule of the present disclosure comprises an amino acid sequence having at least about 95%, at least about 98%, at least about 99%, or 100%, amino acid sequence identity to amino acids 384 to 717 of the H77 amino acid sequence depicted in FIG. 1A-IC; and can have a length of about 334 amino acids (e.g., from about 330 amino acids to about 340 amino acids), where such an E2 polypeptide is referred to as a “soluble” H77 E2 polypeptide.

### Genotype 2—J6

In some cases, an E2 polypeptide that is encoded by an RNA molecule of the present disclosure is a genotype 2 E2 polypeptide and comprises an amino acid sequence having at least about 95%, at least about 98%, at least about 99%, or 100%, amino acid sequence identity to amino acids 384 to 751 of the amino acid sequence identified in FIG. 2A-2B as “D00944 (HC-J6)” (also referred to herein as “J6”); and can have a length of about 368 amino acids (e.g., from 365 amino acids to 370 amino acids); where such a polypeptide is referred to as an “J6 E2 polypeptide.” In some cases, an E2 polypeptide that is encoded by an RNA molecule of the present disclosure comprises an amino acid sequence having at least about 95%, at least about 98%, at least about 99%, or 100%, amino acid sequence identity to amino acids 384 to 661 of the J6 amino acid sequence depicted in FIG. 2A-2B; and can have a length of about 278 amino acids (e.g., from about 275 amino acids to about 280 amino acids), where such an E2 polypeptide is referred to as a “soluble” J6 E2 polypeptide. In some cases, an E2 polypeptide that is encoded by an RNA molecule of the present disclosure comprises an amino acid sequence having at least about 95%, at least about 98%, at least about 99%, or 100%, amino acid sequence identity to amino acids 384 to 717 of the J6 amino acid sequence depicted in FIG. 2A-2B; and can have a length of about 334 amino acids (e.g., from about 330 amino acids to about 340 amino acids), where such an E2 polypeptide is referred to as a “soluble” J6 E2 polypeptide.

### Genotype 3—S52

In some cases, an E2 polypeptide that is encoded by an RNA molecule of the present disclosure is a genotype 3 E2 polypeptide and comprises an amino acid sequence having at least about 95%, at least about 98%, at least about 99%, or 100%, amino acid sequence identity to amino acids 385 to 750 of the amino acid sequence identified in FIG. 3A-3C as “ADF96232 (S52)” (also referred to herein as “S52”); and can have a length of about 366 amino acids (e.g., from about 363 amino acids to about 370 amino acids; where such a polypeptide is referred to as an “S52 E2 polypeptide.”. In some cases, an E2 polypeptide that is encoded by an RNA molecule of the present disclosure comprises an amino acid sequence having at least about 95%, at least about 98%, at least about 99%, or 100%, amino acid sequence identity to amino acids 385 to 661 of the S52 amino acid sequence depicted in FIG. 3A-3C; and can have a length of about 278 amino acids (e.g., from about 275 amino acids to about 280 amino acids), where such an E2 polypeptide is referred to as a “soluble” S52 E2 polypeptide. In some cases, an E2 polypeptide that is encoded by an RNA molecule of the present disclosure comprises an amino acid sequence having at least about 95%, at least about 98%, at least about 99%, or 100%, amino acid sequence identity to amino acids 385 to 717 of the S52 amino acid sequence depicted in FIG. 3A-3C; and can have a length of about 334 amino acids (e.g., from about 330 amino acids to about 340 amino acids), where such an E2 polypeptide is referred to as a “soluble” S52 E2 polypeptide.

### E1 Polypeptides

An HCV E1 polypeptide that is encoded by an RNA molecule of the present disclosure can have a length of from about 100 amino acids (aa) to about 150 aa, from about 150 aa to about 175 aa, from about 175 aa to about 195 aa, from about 131 aa to about 175 aa, or from about 175 aa to about 193 aa. In some cases, an HCV E1 polypeptide that is encoded by an RNA molecule of the present disclosure is an HCV E1 ectodomain polypeptide. In some cases, an HCV E1 polypeptide that is encoded by an RNA molecule of the present disclosure is a full-length HCV E1 polypeptide.

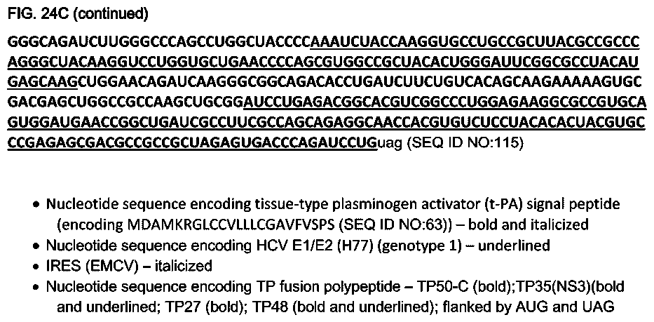

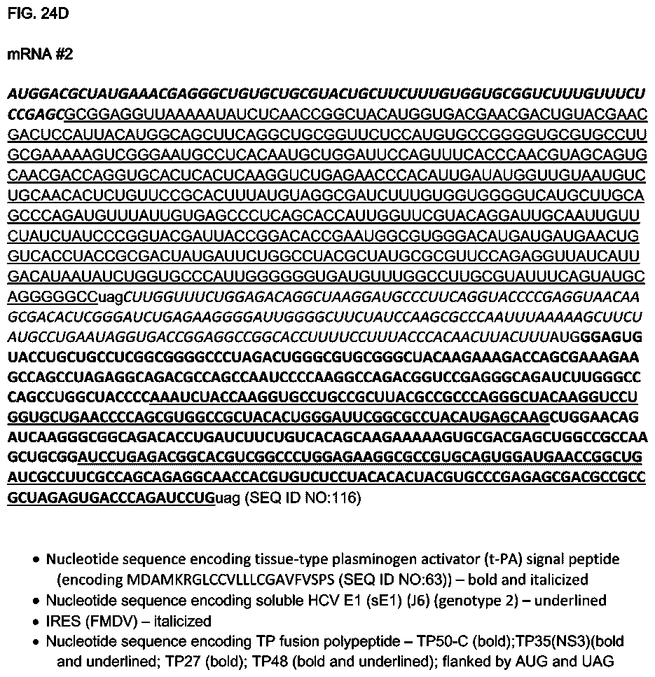

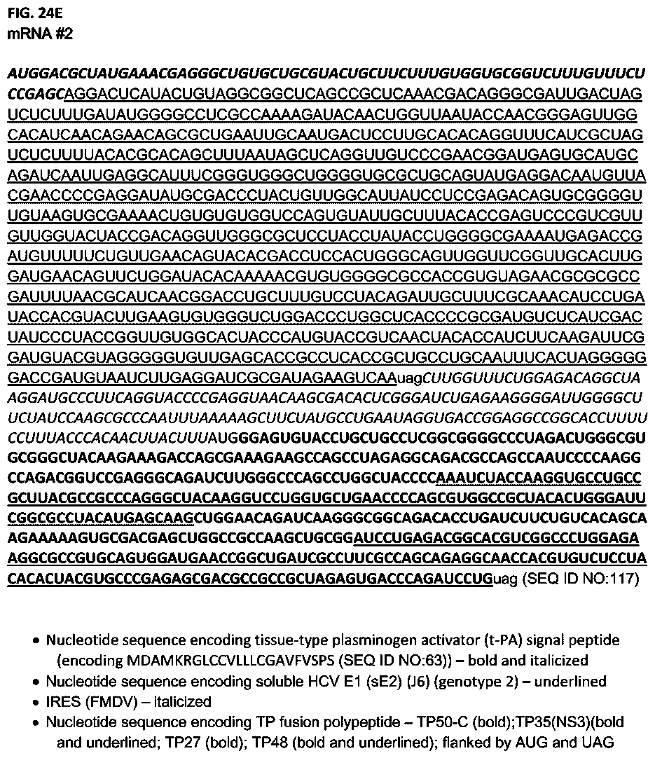

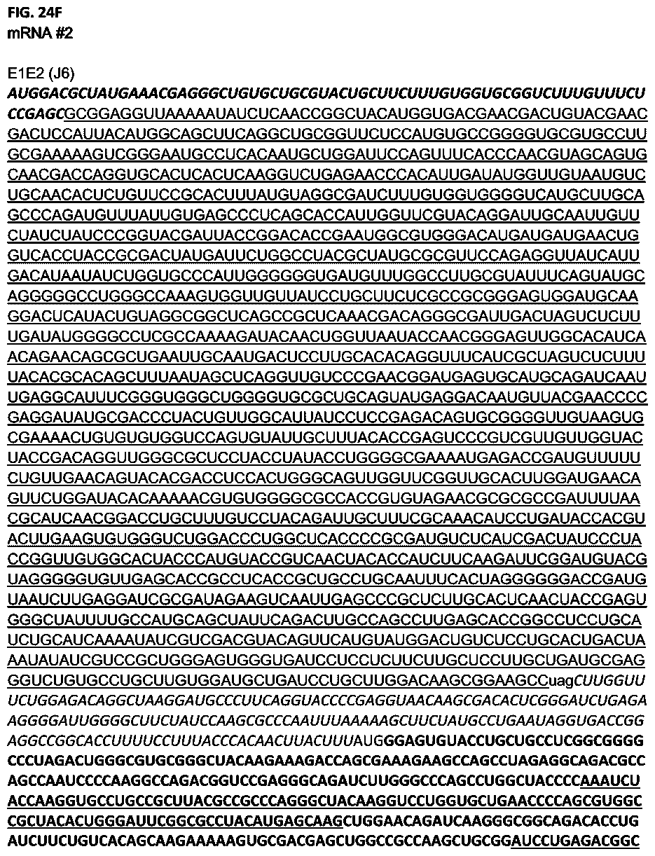

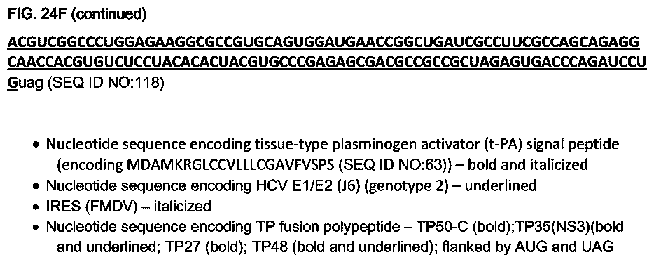

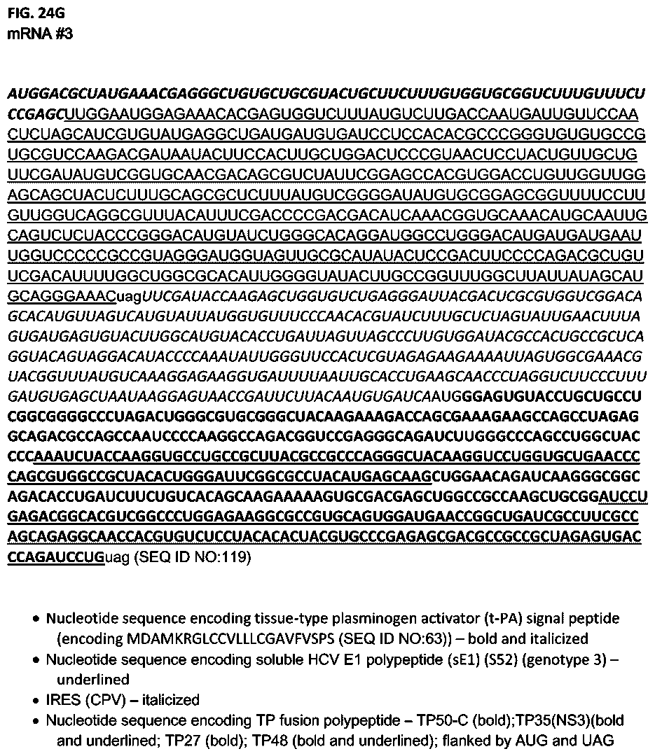

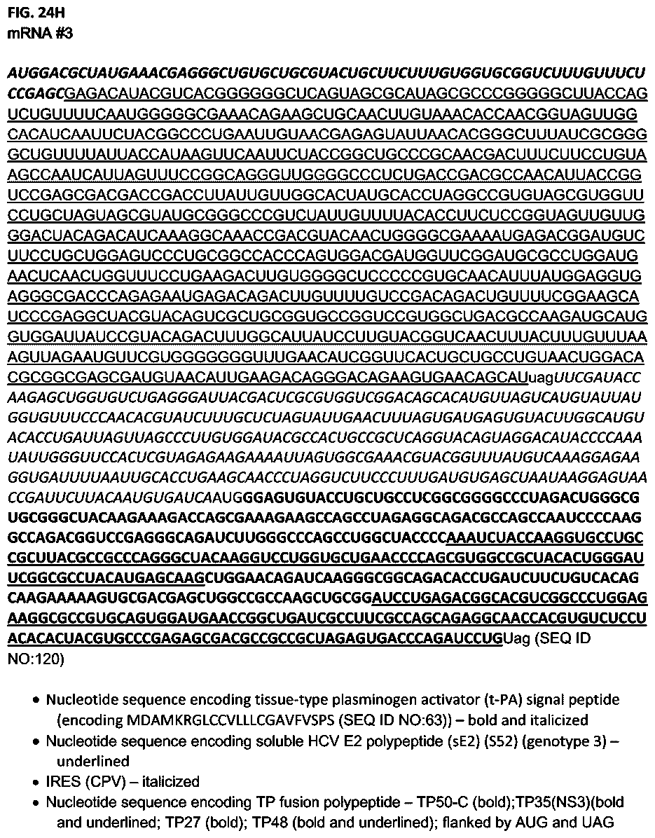

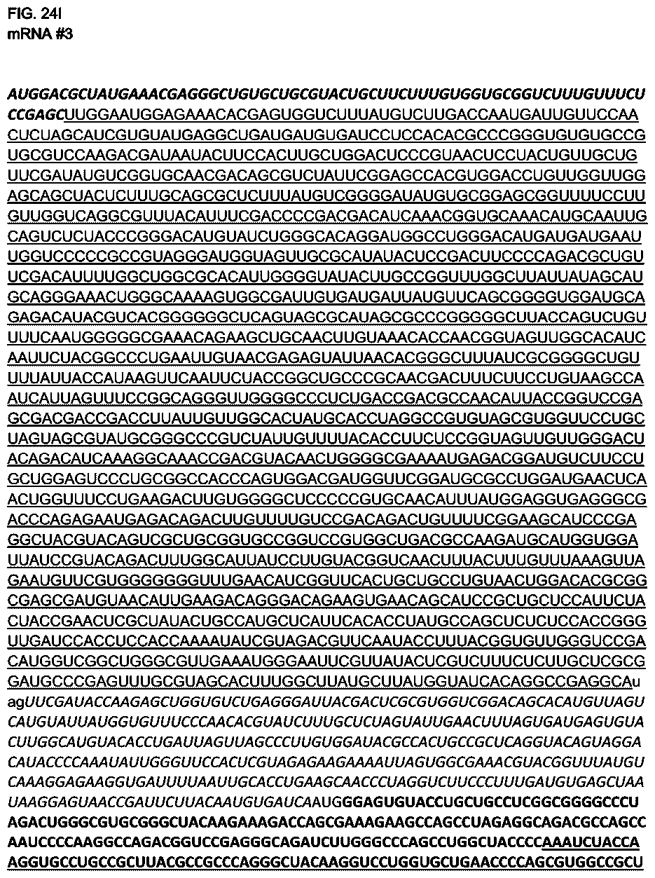

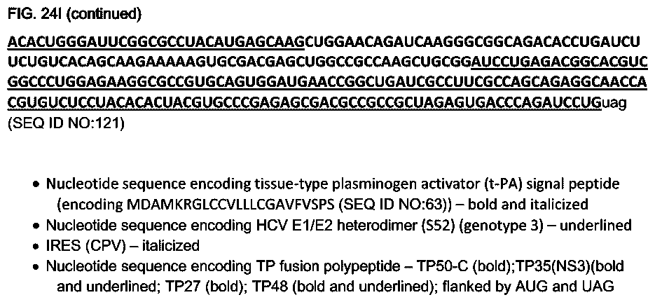

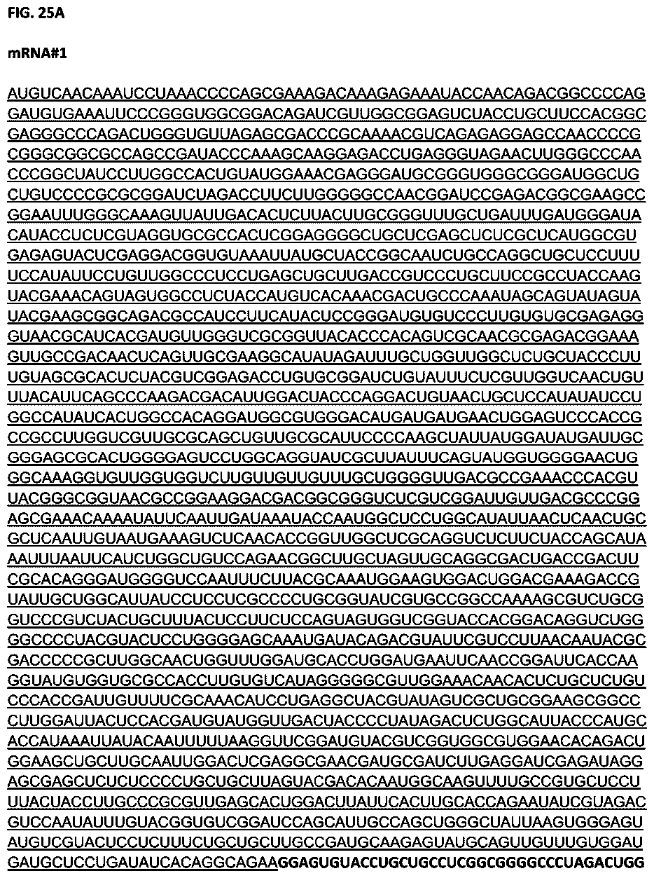

In FIG. 1A-1C, the amino acid sequence of E1 is amino acid 192 to amino acid 383. In FIG. 2A-2C, the amino acid sequence of E1 is amino acid 192 to amino acid 383. In FIG. 3A-3C, the amino acid sequence of E1 is amino acid 192 to amino acid 384. In FIG. 20A-20B, the amino acid sequence of E1 is amino acid 192 to amino acid 383. Amino acids at around 170 through approximately 191 serve as a signal sequence for E1. As used herein, “E1 polypeptide” includes a precursor E1 protein, including the signal sequence; includes a mature E1 polypeptide which lacks this sequence; and includes an E1 polypeptide with a heterologous signal sequence. An E1 polypeptide can include a C-terminal membrane anchor sequence which occurs at approximately amino acid positions 360-383 (see, e.g., WO 96/04301). In some cases, a suitable E1 polypeptide lacks a C-terminal portion that includes a transmembrane region. For example, in some cases, a suitable E1 polypeptide lacks the C-terminal portion from amino acid 330 to amino acid 384, or from amino acid 360 to amino acid 384. E1 polypeptides can be an E1 polypeptide of any genotype, subtype or isolate of HCV. E1 polypeptides of genotype 1, E1 polypeptides of genotype 2, and E1 polypeptides of genotype 3 can be encoded by an RNA molecule of the present disclosure. An E1 polypeptide that lacks a C-terminal region, e.g., a C-terminal membrane anchor sequence, is also referred to herein as a “soluble” E1 polypeptide. A non-limiting example of a nucleotide sequence encoding a soluble E1 polypeptide is provided in FIG. 25G. In some cases, a soluble E1 polypeptide comprises amino acids 192-352 of any one of the amino acid sequences depicted in FIG. 1A-1C, FIG. 2A-2B, FIG. 3A-3C, or FIG. 20A-20B. In some cases, a soluble E1 polypeptide comprises amino acids 192-367 of any one of the amino acid sequences depicted in FIG. 1A-IC, FIG. 2A-2B, FIG. 3A-3C, or FIG. 20A-20B; and lacks the C-terminal portion, e.g., from amino acids 352 to 383, from amino acids 367 to 383, or from amino acids 360 to 383. A non-limiting example of a nucleotide sequence encoding a soluble E1 polypeptide is provided in FIG. 24A. See, e.g., Guest et al. (2021) Proc. Natl. Acad. Sci. USA 18:e2015149118; Cao et al. (2019) PLoS Pathogens 15:e1007759.

An E1 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 1A-1C, FIG. 2A-2C, FIG. 3A-3C, or FIG. 20A-20B.

An E1 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 1A-1C. For example, an E1 polypeptide of genotype 1A that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 1A and depicted in FIG. 1A-1C. For example, an E1 polypeptide of genotype 1B that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 1B and depicted in FIG. 1A-1C. For example, an E1 polypeptide of genotype 1C that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 1C and depicted in FIG. 1A-1C.

An E1 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 2A-2C. For example, an E1 polypeptide of genotype 2A that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 2A and depicted in FIG. 2A-2C. For example, an E1 polypeptide of genotype 2B that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of an amino acid sequence identified as 2B and depicted in FIG. 2A-2C.

An E1 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the consensus E1 polypeptide amino acid sequence depicted in FIG. 3A-3C.

An E1 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to an amino acid sequence of an E1 polypeptide depicted in FIG. 20A-20B. For example, an E1 polypeptide of genotype 7A that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to amino acids 192-383 of the amino acid sequence depicted in FIG. 20A-20B.

An E1 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the amino acid sequence of a genotype 4 E1 polypeptide depicted in FIG. 26B, where the E1 polypeptide amino acid sequence is underlined in FIG. 26B. An example of a nucleotide sequence encoding an E1 polypeptide of genotype 4 is provided in FIG. 26A, where the nucleotide sequence encoding the E2 polypeptide is underlined.

An E1 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the amino acid sequence of a genotype 5 E1 polypeptide depicted in FIG. 27B, where the E1 polypeptide amino acid sequence is underlined in FIG. 27B. An example of a nucleotide sequence encoding an E1 polypeptide of genotype 5 is provided in FIG. 27A, where the nucleotide sequence encoding the E2 polypeptide is underlined.

An E1 polypeptide that is encoded by an RNA molecule of the present disclosure can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the amino acid sequence of a genotype 6 E1 polypeptide depicted in FIG. 28B, where the E1 polypeptide amino acid sequence is underlined in FIG. 28B. An example of a nucleotide sequence encoding an E1 polypeptide of genotype 6 is provided in FIG. 28A, where the nucleotide sequence encoding the E2 polypeptide is underlined.

### Genotype 1—H77

In some cases, an E1 polypeptide that is encoded by an RNA molecule of the present disclosure is a genotype 1 E1 polypeptide and comprises an amino acid sequence having at least about 95%, at least about 98%, at least about 99%, or 100%, amino acid sequence identity to amino acids 192 to 383 of the amino acid sequence identified in FIG. 1A-1C as “NP_671491 (H77)” (also referred to herein as “H77”); and can have a length of about 192 amino acids (e.g., from 190 amino acids to 195 amino acids); where such a polypeptide is referred to as an “H77 E1 polypeptide.” In some cases, an E1 polypeptide that is encoded by an RNA molecule of the present disclosure comprises an amino acid sequence having at least about 95%, at least about 98%, at least about 99%, or 100%, amino acid sequence identity to amino acids 192 to 352 of the H77 amino acid sequence depicted in FIG. 1A-IC; and can have a length of about 161 amino acids (e.g., from about 158 amino acids to about 165 amino acids), where such an E1 polypeptide is referred to as a “soluble” H77 E1 polypeptide. In some cases, an E1 polypeptide that is encoded by an RNA molecule of the present disclosure comprises an amino acid sequence having at least about 95%, at least about 98%, at least about 99%, or 100%, amino acid sequence identity to amino acids 192 to 367 of the H77 amino acid sequence depicted in FIG. 1A-1C; and can have a length of about 176 amino acids (e.g., from about 174 amino acids to about 180 amino acids), where such an E1 polypeptide is referred to as a “soluble” H77 E1 polypeptide. In some cases, an E1 polypeptide that is encoded by an RNA molecule of the present disclosure comprises an amino acid sequence having at least about 95%, at least about 98%, at least about 99%, or 100%, amino acid sequence identity to amino acids 192 to 359 of the H77 amino acid sequence depicted in FIG. 1A-1C; and can have a length of about 168 amino acids (e.g., from about 165 amino acids to about 172 amino acids), where such an E1 polypeptide is referred to as a “soluble” H77 E1 polypeptide. In some cases, an E1 polypeptide that is encoded by an RNA molecule of the present disclosure comprises an amino acid sequence having at least about 95%, at least about 98%, at least about 99%, or 100%, amino acid sequence identity to amino acids 192 to 331 of the H77 amino acid sequence depicted in FIG. 1A-1C; and can have a length of about 140 amino acids (e.g., from about 135 amino acids to about 145 amino acids), where such an E1 polypeptide is referred to as a “soluble” H77 E1 polypeptide.

### Genotype 2—J6

In some cases, an E1 polypeptide that is encoded by an RNA molecule of the present disclosure is a genotype 2 E1 polypeptide and comprises an amino acid sequence having at least about 95%, at least about 98%, at least about 99%, or 100%, amino acid sequence identity to amino acids 192 to 383 of the amino acid sequence identified in FIG. 2A-2B as “D00944 (HC-J6)” (also referred to herein as “J6”); and can have a length of about 192 amino acids (e.g., from 190 amino acids to 195 amino acids); where such a polypeptide is referred to as a “J6 E1 polypeptide.”. In some cases, an E1 polypeptide that is encoded by an RNA molecule of the present disclosure comprises an amino acid sequence having at least about 95%, at least about 98%, at least about 99%, or 100%, amino acid sequence identity to amino acids 192 to 352 of the J6 amino acid sequence depicted in FIG. 2A-2B; and can have a length of about 161 amino acids (e.g., from about 158 amino acids to about 165 amino acids), where such an E1 polypeptide is referred to as a “soluble” J6 E1 polypeptide. In some cases, an E1 polypeptide that is encoded by an RNA molecule of the present disclosure comprises an amino acid sequence having at least about 95%, at least about 98%, at least about 99%, or 100%, amino acid sequence identity to amino acids 192 to 367 of the J6 amino acid sequence depicted in FIG. 2A-2B; and can have a length of about 176 amino acids (e.g., from about 174 amino acids to about 180 amino acids), where such an E1 polypeptide is referred to as a “soluble” J6 E1 polypeptide. In some cases, an E1 polypeptide that is encoded by an RNA molecule of the present disclosure comprises an amino acid sequence having at least about 95%, at least about 98%, at least about 99%, or 100%, amino acid sequence identity to amino acids 192 to 359 of the J6 amino acid sequence depicted in FIG. 2A-2B; and can have a length of about 168 amino acids (e.g., from about 165 amino acids to about 172 amino acids), where such an E1 polypeptide is referred to as a “soluble” J6 E1 polypeptide. In some cases, an E1 polypeptide that is encoded by an RNA molecule of the present disclosure comprises an amino acid sequence having at least about 95%, at least about 98%, at least about 99%, or 100%, amino acid sequence identity to amino acids 192 to 331 of the J6 amino acid sequence depicted in FIG. 2A-2B; and can have a length of about 140 amino acids (e.g., from about 135 amino acids to about 145 amino acids), where such an E1 polypeptide is referred to as a “soluble” J6 E1 polypeptide.

### Genotype 3—S52

In some cases, an E1 polypeptide that is encoded by an RNA molecule of the present disclosure is a genotype 3 E1 polypeptide and comprises an amino acid sequence having at least about 95%, at least about 98%, at least about 99%, or 100%, amino acid sequence identity to amino acids 192 to 384 of the amino acid sequence identified in FIG. 3A-3C as “ADF96232 (S52)” (also referred to herein as “S52”); and can have a length of about 193 amino acids (e.g., from 190 amino acids to 196 amino acids); where such a polypeptide is referred to as an “S52 E1 polypeptide.” In some cases, an E1 polypeptide that is encoded by an RNA molecule of the present disclosure comprises an amino acid sequence having at least about 95%, at least about 98%, at least about 99%, or 100%, amino acid sequence identity to amino acids 192 to 352 of the S52 amino acid sequence depicted in FIG. 3A-3C; and can have a length of about 161 amino acids (e.g., from about 158 amino acids to about 165 amino acids), where such an E1 polypeptide is referred to as a “soluble” S52 E1 polypeptide. In some cases, an E1 polypeptide that is encoded by an RNA molecule of the present disclosure comprises an amino acid sequence having at least about 95%, at least about 98%, at least about 99%, or 100%, amino acid sequence identity to amino acids 192 to 367 of the S52 amino acid sequence depicted in FIG. 3A-3C; and can have a length of about 176 amino acids (e.g., from about 174 amino acids to about 180 amino acids), where such an E1 polypeptide is referred to as a “soluble” S52 E1 polypeptide. In some cases, an E1 polypeptide that is encoded by an RNA molecule of the present disclosure comprises an amino acid sequence having at least about 95%, at least about 98%, at least about 99%, or 100%, amino acid sequence identity to amino acids 192 to 359 of the S52 amino acid sequence depicted in FIG. 3A-3C; and can have a length of about 168 amino acids (e.g., from about 165 amino acids to about 172 amino acids), where such an E1 polypeptide is referred to as a “soluble” S52 E1 polypeptide. In some cases, an E1 polypeptide that is encoded by an RNA molecule of the present disclosure comprises an amino acid sequence having at least about 95%, at least about 98%, at least about 99%, or 100%, amino acid sequence identity to amino acids 192 to 331 of the S52 amino acid sequence depicted in FIG. 3A-3C; and can have a length of about 140 amino acids (e.g., from about 135 amino acids to about 145 amino acids), where such an E1 polypeptide is referred to as a “soluble” S52 E1 polypeptide.

### HCV Core

In some cases, the polypeptide-encoding region of an RNA of the present disclosure comprises a nucleotide sequence encoding HCV core protein. In some cases, the polypeptide-encoding region encodes a core-E1-E2 polyprotein, which is then processed intracellularly to produce core, E1, and E2 (where the E1 and E2 polypeptides can form a heterodimer). Inclusion of an HCV core protein-encoding nucleotide sequence may result in formation of a viral-like particle (VLP) comprising core, E1, and E2.

HCV core protein has 191 amino acids and can be divided into three domains on the basis of hydrophobicity: domain 1 (residues 1-117) contains mainly basic residues with two short hydrophobic regions; domain 2 (residues 118-174) is less basic and more hydrophobic and its C-terminus is at the end of p21; domain 3 (residues 175-191) is highly hydrophobic and acts as a signal sequence for E1 envelope protein.

Amino acid sequences of HCV core proteins of various genotypes are known in the art. See, e.g., the amino acid sequences depicted in FIG. 1A-1C, FIG. 2A-2C, FIG. 3A-3C, FIG. 20A-20B, FIG. 26B, FIG. 27B, and FIG. 28B. In some cases, an HCV core protein comprises an amino acid sequence having at least 50%, at least 60%, at least 70%, at least 75%, at least 80%, at least 85%, at least 90%, at least 95%, at least 98%, at least 98%, or 100%, amino acid sequence identity to amino acids 1-191 of any one of the core-E1-E2 amino acid sequences depicted in FIG. 1A-1C, FIG. 2A-2C, FIG. 3A-3C, FIG. 20A-20B, FIG. 26B, FIG. 27B, and FIG. 28B. In some cases, an HCV core protein has a length of from about 170 amino acids to about 191 amino acids; e.g., in some cases, an HCV core protein has a length of from about 170 amino acids to about 175 amino acids, from about 175 amino acids to about 180 amino acids, from about 180 amino acids to about 185 amino acids, or from about 185 to about 191 amino acids.

The core protein can be of any HCV genotype. For example, the core protein can be a genotype 1a core protein, a genotype 1b core protein, a genotype 2 core protein, a genotype 3 core protein, a genotype 4 core protein, a genotype 5 core protein, a genotype 6 core protein, etc.

In some cases, a composition of the present disclosure comprises an RNA molecule comprising: i) a first nucleotide sequence encoding an HCV core polypeptide; ii) a second nucleotide sequence encoding an HCV E1 polypeptide; and iii) a third nucleotide sequence encoding an HCV E2 polypeptide. The first, second, and third nucleotide sequence can be separated by IRESs, where the IRESs can be the same or different.

In some cases, a composition of the present disclosure comprises a nucleotide sequence encoding a core-E1-E2 polyprotein. In some cases, a composition of the present disclosure comprises a nucleotide sequence encoding HCV core protein, a nucleotide sequence encoding an HCV E1 polypeptide, and a nucleotide sequence encoding an HCV E2 polypeptide, where the nucleotide sequence encoding the core protein and the E1 protein are separated by an IRES and where the nucleotide sequence encoding the E1 protein and the E2 protein are separated by an IRES. In some cases, a composition of the present disclosure comprises: i) a first nucleic acid comprising a nucleotide sequence encoding a core-E1-E2 polyprotein; and ii) a second nucleic acid comprising a nucleotide sequence encoding a fusion polypeptide comprising two or more TPs. In some cases, a composition of the present disclosure comprises: i) a first nucleic acid comprising a nucleotide sequence encoding a core-E1-E2 polyprotein; and ii) a second nucleic acid comprising one or more nucleotides encoding one or more TPs.

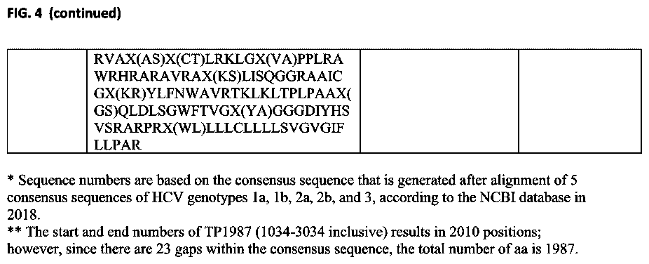

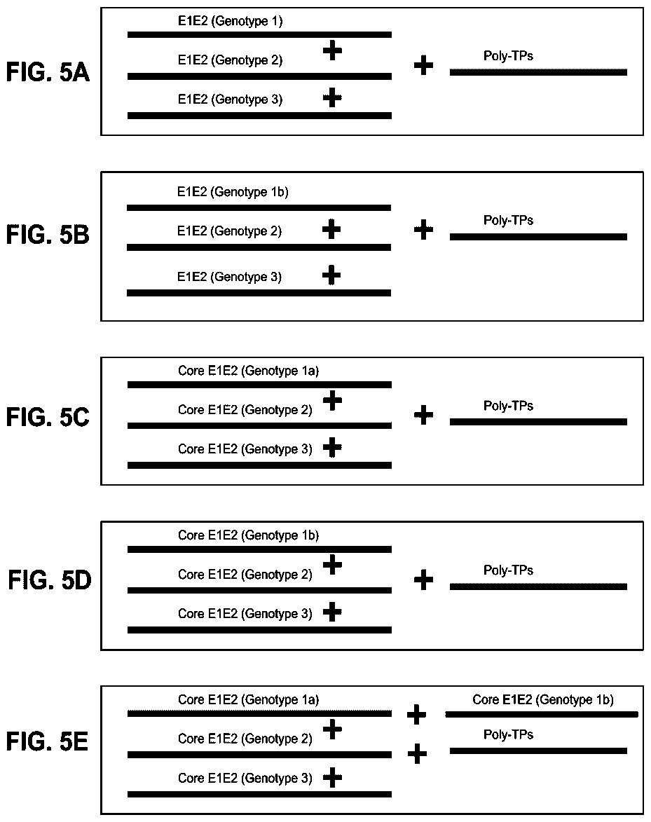

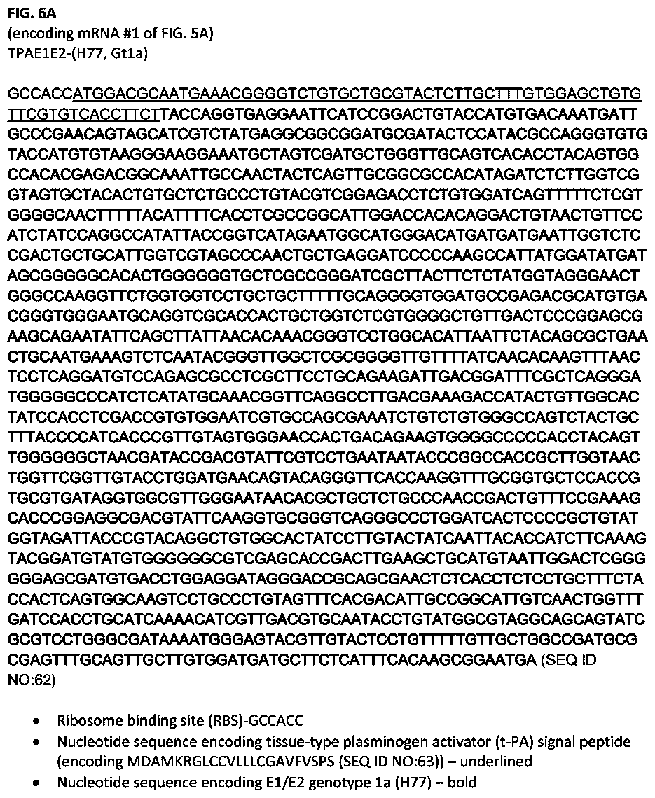

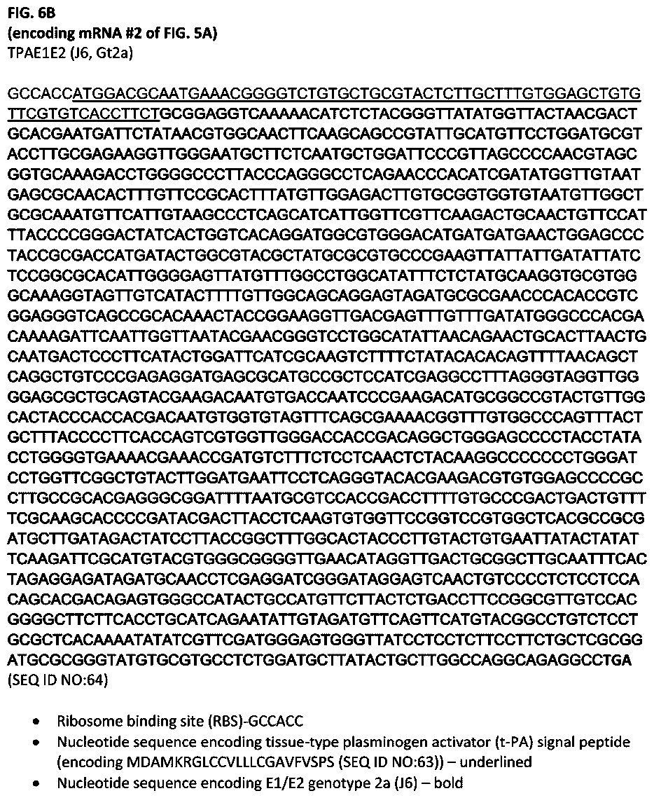

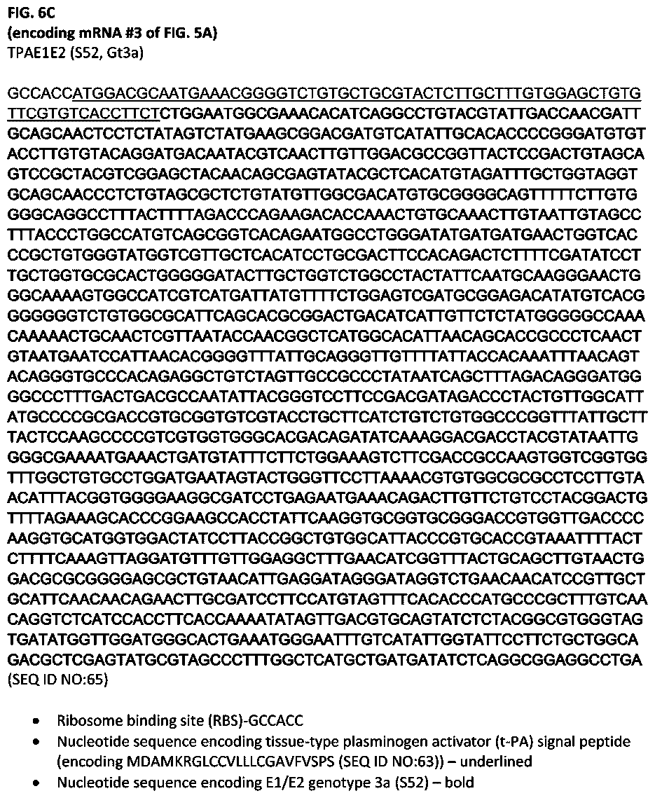

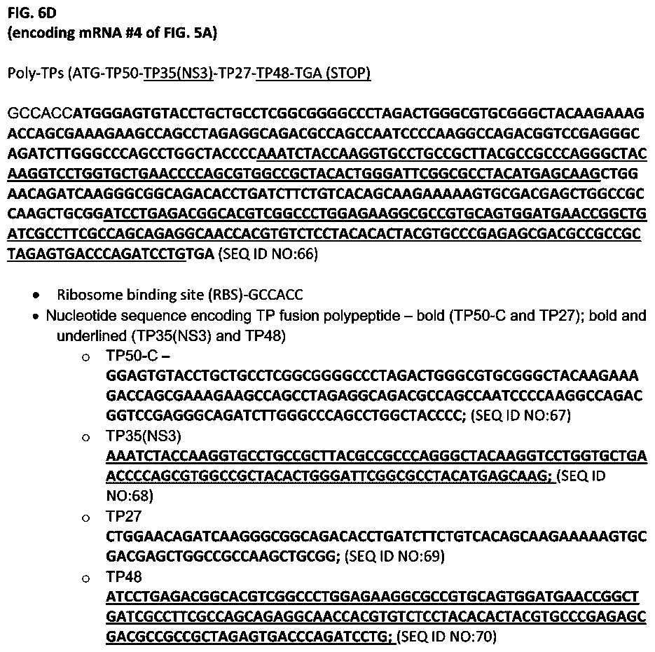

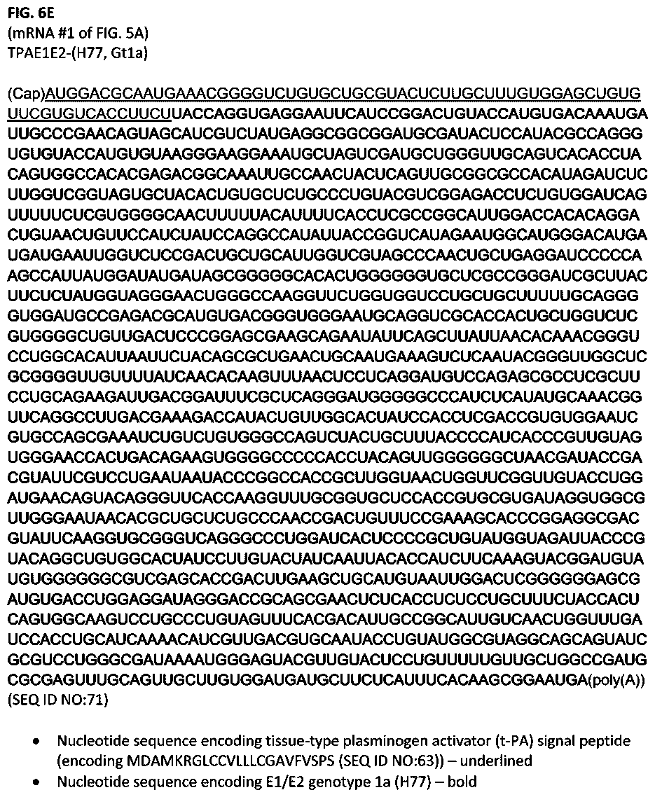

For example, as depicted schematically in FIG. 5C, in some cases, a composition of the present disclosure comprises: a) a first RNA molecule comprising a nucleotide sequence(s) encoding HCV core, HCV E1, and HCVE2 of genotype 1a; b) a second RNA molecule comprising a nucleotide sequence(s) encoding HCV core, HCV E1, and HCVE2 of genotype 2; c) a third RNA molecule comprising a nucleotide sequence(s) encoding HCV core, HCV E1, and HCVE2 of genotype 3; and d) a fourth RNA molecule comprising one or more nucleotide sequences encoding one or more heterologous polypeptides (e.g., one or more TPs) or a heterologous fusion polypeptide (e.g., a fusion polypeptide comprising two or more (e.g., 1, 2, 3, 4, 5, 6, 7, 8, 9, or 10 TPs).

As another example, as depicted schematically in FIG. 5D, in some cases, a composition of the present disclosure comprises: a) a first RNA molecule comprising a nucleotide sequence(s) encoding HCV core, HCV E1, and HCVE2 of genotype 1b; b) a second RNA molecule comprising a nucleotide sequence(s) encoding HCV core, HCV E1, and HCVE2 of genotype 2; c) a third RNA molecule comprising a nucleotide sequence(s) encoding HCV core, HCV E1, and HCVE2 of genotype 3; and d) a fourth RNA molecule comprising one or more nucleotide sequences encoding one or more heterologous polypeptides (e.g., one or more TPs) or a heterologous fusion polypeptide (e.g., a fusion polypeptide comprising two or more (e.g., 1, 2, 3, 4, 5, 6, 7, 8, 9, or 10 TPs).

As another example, as depicted schematically in FIG. 5E, in some cases, a composition of the present disclosure comprises: a) a first RNA molecule comprising a nucleotide sequence(s) encoding HCV core, HCV E1, and HCVE2 of genotype 1a; b) a second RNA molecule comprising a nucleotide sequence(s) encoding HCV core, HCV E1, and HCVE2 of genotype 2; c) a third RNA molecule comprising a nucleotide sequence(s) encoding HCV core, HCV E1, and HCVE2 of genotype 3; d) a fourth RNA molecule comprising a nucleotide sequence(s) encoding HCV core, HCV E1, and HCVE2 of genotype 1b; b) a second RNA molecule comprising a nucleotide sequence(s) encoding HCV core, HCV E1, and HCVE2 of genotype 2 and e) a fifth RNA molecule comprising one or more nucleotide sequences encoding one or more heterologous polypeptides (e.g., one or more TPs) or a heterologous fusion polypeptide (e.g., a fusion polypeptide comprising two or more (e.g., 1, 2, 3, 4, 5, 6, 7, 8, 9, or 10 TPs).

In other instances, a separate RNA molecule encodes HCV core protein. For example, in some cases, a composition of the present disclosure comprises: a) a first RNA molecule comprising a nucleotide sequence encoding HCV core; b) a second RNA molecule comprising a nucleotide sequence encoding HCV E1 and/or E2; and c) a third RNA molecule comprising one or more nucleotide sequences encoding one or more heterologous polypeptides (e.g., one or more TPs) or a heterologous fusion polypeptide (e.g., a fusion polypeptide comprising two or more (e.g., 1, 2, 3, 4, 5, 6, 7, 8, 9, or 10 TPs).

### Genotype 1—H77

In some cases, an HCV Core polypeptide that is encoded by an RNA molecule of the present disclosure is a genotype 1 Core polypeptide and comprises an amino acid sequence having at least about 95%, at least about 98%, at least about 99%, or 100%, amino acid sequence identity to amino acids 1 to 191 of the amino acid sequence identified in FIG. 1A-1C as “NP_671491 (H77)” (also referred to herein as “H77”); and can have a length of about 191 amino acids (e.g., from 189 amino acids to 195 amino acids); where such a polypeptide is referred to as an “H77 Core polypeptide.” In some cases, an RNA molecule of the present disclosure comprises a nucleotide sequence encoding a Core-E1-E2 polyprotein. In some cases, the Core-E1-E2 polyprotein comprises: i) an H77 Core polypeptide; ii) an H77 E1 polypeptide, as described above; and iii) an H77 E2 polypeptide, as described above. For example, in some cases, the Core-E1-E2 polyprotein is a genotype 1 Core-E1-E2 polyprotein and comprises an amino acid sequence having at least about 95%, at least about 98%, at least about 99%, or 100%, amino acid sequence identity to amino acids 1 to 746 of the H77 amino acid sequence depicted in FIG. 1A-1C.

### Genotype 2—J6

In some cases, an HCV Core polypeptide that is encoded by an RNA molecule of the present disclosure is a genotype 2 Core polypeptide and comprises an amino acid sequence having at least about 95%, at least about 98%, at least about 99%, or 100%, amino acid sequence identity to amino acids 1 to 191 of the amino acid sequence identified in FIG. 2A-2B as “D00944 (HC-J6)” (also referred to herein as “J6”); and can have a length of about 191 amino acids (e.g., from 189 amino acids to 195 amino acids); where such a polypeptide is referred to as a “J6 Core polypeptide.” In some cases, an RNA molecule of the present disclosure comprises a nucleotide sequence encoding a Core-E1-E2 polyprotein. In some cases, the Core-E1-E2 polyprotein comprises: i) a J6 Core polypeptide, as described above; and ii) a J6 E1 polypeptide; and iii) a J6 E2 polypeptide, as described above. For example, in some cases, the Core-E1-E2 polyprotein is a genotype 2 Core-E1-E2 polyprotein and comprises an amino acid sequence having at least about 95%, at least about 98%, at least about 99%, or 100%, amino acid sequence identity to amino acids 1 to 751 of the J6 amino acid sequence depicted in FIG. 2A-2B.

### Genotype 3—S52

In some cases, an HCV Core polypeptide that is encoded by an RNA molecule of the present disclosure is a genotype 3 Core polypeptide and comprises an amino acid sequence having at least about 95%, at least about 98%, at least about 99%, or 100%, amino acid sequence identity to amino acids 1 to 191 of the amino acid sequence identified in FIG. 3A-3C as “ADF96232 (S52)” (also referred to herein as “S52”); and can have a length of about 191 amino acids (e.g., from 189 amino acids to 195 amino acids); where such a polypeptide is referred to as an “S52 Core polypeptide.”

In some cases, an RNA molecule of the present disclosure comprises a nucleotide sequence encoding a Core-E1-E2 polyprotein. In some cases, the Core-E1-E2 polyprotein comprises: i) an S52 Core polypeptide, as described above; and ii) an S52 6 E1 polypeptide; and iii) an S52 E2 polypeptide, as described above. For example, in some cases, the Core-E1-E2 polyprotein is a genotype 3 Core-E1-E2 polyprotein and comprises an amino acid sequence having at least about 95%, at least about 98%, at least about 99%, or 100%, amino acid sequence identity to amino acids 1 to 750 of the S52 amino acid sequence depicted in FIG. 3A-3C.

### TPs

In some cases, the polypeptide-encoding region of an RNA of the present disclosure comprises a nucleotide sequence encoding a T-cell epitope polypeptide (a polypeptide other than an HCV E1 or E2 polypeptide) comprising T-cell epitopes (e.g., CD4+ and/or CD8+ T-cell epitopes that are conserved between or among two or more HCV genotypes and that are presented through one or multiple HLA alleles common within the human population) present in an HCV protein other than E1 and E2. Such T-cell epitope polypeptides are referred to herein as “TPs.”

Thus, a TP encoded by an RNA molecule of the present disclosure comprises multiple (e.g., 2, 3, 4, or 5, or more than 5) CD4+ and/or CD8+ T-cell epitopes that are conserved between or among two or more HCV genotypes and that are presented through one or multiple HLA alleles common within the human population. In some cases, a CD8 epitope present in a TP is presented through a single HLA allele (a single HLA haplotype). In some cases, a CD4 epitope present in a TP is presented through multiple different HLA alleles (multiple different HLA haplotypes).

A TP encoded by an RNA molecule of the present disclosure exhibits one or more of the following features: 1) displays immunodominant epitopes; 2) displays epitopes that are conserved between or among two or more different HCV genotypes; 3) displayed through human leukocyte antigen (HLA) alleles that are common within the human population; 4) good yields by chemical synthesis; and 5) good solubility in a buffered aqueous solution.

In some cases, a TP encoded by an RNA molecule of the present disclosure displays 1, 2, 3, 4, 5, or from 5 to 10, different epitopes displayed by an HCV polypeptide other than E1 and E2. In some cases, the epitopes are immunodominant epitopes.

In some cases, a TP encoded by an RNA molecule of the present disclosure displays one or more epitopes that are conserved between or among two or more HCV genotypes. For example, in some cases, a TP suitable for inclusion in a composition of the present disclosure displays 1, 2, 3, 4, 5, or from 5 to 10, epitopes that are conserved between HCV genotype 1 and HCV genotype 3. As another example, in some cases, a TP encoded by an RNA molecule of the present disclosure displays 1, 2, 3, 4, 5, or from 5 to 10, epitopes that are conserved between HCV genotype 1 and HCV genotype 2. As another example, in some cases, a TP encoded by an RNA molecule of the present disclosure displays 1, 2, 3, 4, 5, or from 5 to 10, epitopes that are conserved among HCV genotypes 1, 2, and 3. As another example, in some cases, a TP encoded by an RNA molecule of the present disclosure displays 1, 2, 3, 4, 5, or from 5 to 10, epitopes that are conserved among HCV genotypes 1, 2, 3, and 6.

### TP35-NS3 T-Cell Epitope Polypeptides

In some cases, a TP encoded by an RNA molecule of the present disclosure comprises an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: KSTKVPAAYAAQGYKVLVLNPSVAATLGFGAYMSK (SEQ ID NO:102; also referred to herein as “TP35-NS3”); where the TP has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP is part of a fusion polypeptide, as described in detail below.

In some cases, a TP35-NS3 polypeptide encoded by an RNA molecule of the present disclosure comprises the following amino acid sequence: KSTKVPX₁AYX₂X₃QGYX₄VLVLNPSVAATLGFGX₅X₆X₇SX₈ (SEQ ID NO:219), where X₁ is A or V; X₂ is A or V; X₃ is A or S; X₄ is K or N; X₅ is A or S; X₆ is Y or F; X₇ is M or L; and X₈ is K or R; where the TP35-NS3 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP has a length of 35 amino acids. In some cases, the TP is part of a fusion polypeptide, as described in detail below.

In some cases, a TP35-NS3 polypeptide encoded by an RNA molecule of the present disclosure comprises the following amino acid sequence: KSTKVPVAYAAQGYKVLVLNPSVAATLGFGAYLSK (SEQ ID NO:220); where the TP35-NS3 polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP has a length of 35 amino acids. In some cases, the TTP is part of a fusion polypeptide, as described in detail below.

In some cases, a TP35-NS3 polypeptide encoded by an RNA molecule of the present disclosure comprises the following amino acid sequence: KSTKVPAAYASQGYKVLVLNPSVAATLGFGAYMSK (SEQ ID NO:221); where the TP35-NS3 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP has a length of 35 amino acids. In some cases, the TP is part of a fusion polypeptide, as described in detail below.

In some cases, a TP35-NS3 polypeptide encoded by an RNA molecule of the present disclosure comprises the following amino acid sequence: KSTKVPAAYVAQGYNVLVLNPSVAATLGFGSFMSR (SEQ ID NO:222); where the TP35-NS3 polypeptide has a length of from 28 amino acids to 35 amino acids (e.g., the TP has a length of 28 amino acids (aa), 29 aa, 30 aa, 31 aa, 32 aa, 33 aa, 34 aa, or 35 aa). In some cases, the TP has a length of 35 amino acids. In some cases, the TP is part of a fusion polypeptide, as described in detail below.

In some cases, a TP35-NS3 polypeptide encoded by an RNA molecule of the present disclosure comprises the following amino acid sequence: KSTKVPAAYAAQGYKVLVLNPSVAATLGFGAYMSR (SEQ ID NO:223); where the TP35-NS3 polypeptide has a length of from 28 amino acids to 35 amino acids (e.g., the TP has a length of 28 amino acids (aa), 29 aa, 30 aa, 31 aa, 32 aa, 33 aa, 34 aa, or 35 aa). In some cases, the TP has a length of 35 amino acids. In some cases, the TP is part of a fusion polypeptide, as described in detail below.

In some cases, a TP35-NS3 polypeptide encoded by an RNA molecule of the present disclosure comprises the following amino acid sequence: KSTKVPAAYASQGYKVLVLNPSVAATLGFGSYMSK (SEQ ID NO:224); where the TP35-NS3 polypeptide has a length of from 28 amino acids to 35 amino acids (e.g., the TP has a length of 28 amino acids (aa), 29 aa, 30 aa, 31 aa, 32 aa, 33 aa, 34 aa, or 35 aa). In some cases, the TP has a length of 35 amino acids. In some cases, the TP is part of a fusion polypeptide, as described in detail below.

In some cases, a TP35-NS3 polypeptide encoded by an RNA molecule of the present disclosure comprises the following amino acid sequence: KSTKVPAAYASQGYKVLVLNPSVAATLGFGAYMSK (SEQ ID NO:221); where the TP35-NS3 polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP has a length of 35 amino acids. In some cases, the TP is part of a fusion polypeptide, as described in detail below.

A TP35-NS3 epitope polypeptide encoded by an RNA molecule of the present disclosure can include a CD8 epitope, e.g., a stretch of 10 contiguous amino acids having the amino acid sequence AYX₁X₂QGYX₃VL (SEQ ID NO:225), where X₁ is A or V; X₂ is A or S; and X₃ is K or N; and/or a stretch of 11 contiguous amino acids having the amino acid sequence ATLGFGX₁X₂X₃SX₄ (SEQ ID NO:226), where X₁ is A or S; X₂ is Y or F; X₃ is M or L; and X₄ is K or R. In some cases, the CD8 epitope is a stretch of 10 contiguous amino acids having the amino acid sequence AYAAQGYKVL (SEQ ID NO:227). In some cases, the CD8 epitope is a stretch of 10 contiguous amino acids having the amino acid sequence AYASQGYKVL (SEQ ID NO:228). In some cases, the CD8 epitope is a stretch of 10 contiguous amino acids having the amino acid sequence AYVAQGYNVL (SEQ ID NO:229). A TP35-NS3 T-cell epitope polypeptide encoded by an RNA molecule of the present disclosure can include a CD8 epitope, e.g., a stretch of 10 contiguous amino acids having the amino acid sequence AYAAQGYKVL (SEQ ID NO:227) and/or a stretch of 11 contiguous amino acids having the amino acid sequence ATLGFGAYMSK (SEQ ID NO:230). A TP35-NS3 polypeptide encoded by an RNA molecule of the present disclosure can include: i) a stretch of 10 contiguous amino acids having the amino acid sequence AYAAQGYKVL (SEQ ID NO:227); and ii) a stretch of 11 contiguous amino acids having the amino acid sequence ATLGFGAYMSK (SEQ ID NO:330).

A TP35-NS3 polypeptide encoded by an RNA molecule of the present disclosure can include a CD4 epitope, e.g., a stretch of about 15 contiguous amino acids having the amino acid sequence: VLVLNPSVAATLGFG (SEQ ID NO:231).

A TP35-NS3 polypeptide encoded by an RNA molecule of the present disclosure can include: i) a CD4 epitope, e.g., a stretch of about 15 contiguous amino acids having the amino acid sequence: VLVLNPSVAATLGFG (SEQ ID NO:231); ii) a first CD8 epitope, e.g., a stretch of 10 contiguous amino acids having the amino acid sequence AYX₁X₂QGYX₃VL (SEQ ID NO:225), where X₁ is A or V; X₂ is A or S; and X₃ is K or N; and iii) a second CD8 epitope, e.g., a stretch of 11 contiguous amino acids having the amino acid sequence ATLGFGX₁X₂X₃SX₄ (SEQ ID NO:226), where X₁ is A or S; X₂ is Y or F; X₃ is M or L; and X₄ is K or R. A TP35-NS3 polypeptide encoded by an RNA molecule of the present disclosure can include: i) a CD4 epitope, e.g., a stretch of about 15 contiguous amino acids having the amino acid sequence: VLVLNPSVAATLGFG (SEQ ID NO:231); ii) a first CD8 epitope, e.g., a stretch of 10 contiguous amino acids having the amino acid sequence AYAAQGYKVL (SEQ ID NO:227); and iii) a second CD8 epitope, e.g., a stretch of 11 contiguous amino acids having the amino acid sequence ATLGFGAYMSK (SEQ ID NO:230).

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: KSTKVPAAYAAQGYKVLVLNPSVAATLGFGAYMSK (SEQ ID NO:102); and has a length of from 35 amino acids to 40 amino acids (e.g., 35 amino acids (aa), 36 aa, 37 aa, 38 aa, 39 aa, or 40 aa).

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: KSTKVPAAYAAQGYKVLVLNPSVAATLGFGAYMSK (SEQ ID NO:102); and has a length of 35 amino acids. In some cases, a TP encoded by an RNA molecule of the present disclosure comprises the amino acid sequence KSTKVPAAYAAQGYKVLVLNPSVAATLGFGAYMSK (SEQ ID NO:102); and has a length of 35 amino acids.

In some cases, a suitable TP is a TP35-NS3 polypeptide with from 1 amino acid to 5 amino acids removed from the N-terminus and/or from 1 amino acid to 5 amino acids removed from the C-terminus. Thus, e.g., a suitable TP can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: PAAYAAQGYKVLVLNPSVAATLGFGAYMSK (SEQ ID NO:232); and has a length of 30 amino acids. As another example, a suitable TP can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: KSTKVPAAYAAQGYKVLVLNPSVAATLGFG (SEQ ID NO:233); and has a length of 30 amino acids.

### TP50-C T-Cell Epitope Polypeptides

In some cases, a TP encoded by an RNA molecule of the present disclosure comprises an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: GVYLLPRRGPRLGVRATRKTSERSQPRGRRQPIPKARRSEGRSWAQPGYP (SEQ ID NO:101); also referred to herein as “TP50C”); where the TP has a length of from 40 amino acids to 50 amino acids (e.g., the TP has a length of 40 amino acids (aa), 41 aa, 42 aa, 43 aa, 44 aa, 45 aa, 46 aa, 47 aa, 48 aa, 49 aa, or 50 aa). In some cases, the TP is part of a fusion polypeptide, as described in detail below.

In some cases, a TP50-C polypeptide encoded by an RNA molecule of the present disclosure comprises the following amino acid sequence: GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₂ is S, T, H, or A; X₁₂ is A or G; and X₁₃ is Q or K; where the TP has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the TP has a length of 50 amino acids. In some cases, the TP is part of a fusion polypeptide, as described in detail below.

In some cases, a TP50-C polypeptide encoded by an RNA molecule of the present disclosure comprises the following amino acid sequence: GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:235), where X₁ is L or V; X₂ is A; X₃ is T; X₄ is P; X₅ is A or D; X₆ is R; X₇ is R or Q; X₈ is S or P; X₉ is E or T; X₁₀ is R or K; X₁₁ is S, T, H, or A; X₁₂ is A or G; and X₁₃ is Q or K; where the TP has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the TP has a length of 50 amino acids. In some cases, the T-cell epitope polypeptide is part of a fusion polypeptide, as described in detail below.

In some cases, a TP50-C polypeptide encoded by an RNA molecule of the present disclosure comprises the following amino acid sequence: GVYLLPRRGPRLGVRATRKTSERSQPRGRRQPIPKARRPEGRTWAQPGYP (SEQ ID NO:236); where the TTP has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the TP has a length of 50 amino acids. In some cases, the TP is part of a fusion polypeptide, as described in detail below.

In some cases, a TP50-C polypeptide encoded by an RNA molecule of the present disclosure comprises the following amino acid sequence: GVYLLPRRGPRLGVRATRKTSERSQPRGRRQPIPKARRPEGRAWAQPGYP (SEQ ID NO:237); where the TP has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the TP has a length of 50 amino acids. In some cases, the T-cell epitope polypeptide is part of a fusion polypeptide, as described in detail below.

In some cases, a TP50-C polypeptide encoded by an RNA molecule of the present disclosure comprises the following amino acid sequence: GVYLLPRRGPRLGVRATRKTSERSQPRGRRQPIPKDRRSTGKSWGKPGYP (SEQ ID NO:238); where the TP has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the TP has a length of 50 amino acids. In some cases, the TP is part of a fusion polypeptide, as described in detail below.

In some cases, a TP50-C polypeptide encoded by an RNA molecule of the present disclosure comprises the following amino acid sequence: GVYLLPRRGPRLGVRATRKTSERSQPRGRRQPIPKDRRSTGKSWGKPGYP (SEQ ID NO:238); where the TP has a length of from 40 amino acids to 50 amino acids (e.g., the TP has a length of 40 amino acids (aa), 41 aa, 42 aa, 43 aa, 44 aa, 45 aa, 46 aa, 47 aa, 48 aa, 49 aa, or 50 aa). In some cases, the T-cell epitope polypeptide has a length of 50 amino acids. In some cases, the TP is part of a fusion polypeptide, as described in detail below.

In some cases, a TP50-C polypeptide encoded by an RNA molecule of the present disclosure comprises the following amino acid sequence: GVYVLPRRGPRLGVRATRKTSERSQPRGRRQPIPKARRSEGRSWAQPGYP (SEQ ID NO:239); where the TP has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the TP has a length of 50 amino acids. In some cases, the TP is part of a fusion polypeptide, as described in detail below.

In some cases, a TP50-C polypeptide encoded by an RNA molecule of the present disclosure comprises the following amino acid sequence: GVYLLPRRGPRLGVRATRKTSERSQPRGRRQPIPKARRPTGRSWGQPGYP (SEQ ID NO:240); where the TP has a length of from 40 amino acids to 50 amino acids (e.g., the T-cell epitope polypeptide has a length of 40 amino acids (aa), 41 aa, 42 aa, 43 aa, 44 aa, 45 aa, 46 aa, 47 aa, 48 aa, 49 aa, or 50 aa). In some cases, the TP has a length of 50 amino acids. In some cases, the TP is part of a fusion polypeptide, as described in detail below.

In some cases, a TP50-C polypeptide encoded by an RNA molecule of the present disclosure comprises the following amino acid sequence: GVYLLPRRGPRLGVRATRKTSERSQPRGRRQPIPKARQPTGRHWAQPGYP (SEQ ID NO:241); where the TP has a length of from 40 amino acids to 50 amino acids (e.g., the TP e has a length of 40 amino acids (aa), 41 aa, 42 aa, 43 aa, 44 aa, 45 aa, 46 aa, 47 aa, 48 aa, 49 aa, or 50 aa). In some cases, the TP has a length of 50 amino acids. In some cases, the TP is part of a fusion polypeptide, as described in detail below.

In some cases, a TP50-C polypeptide encoded by an RNA molecule of the present disclosure comprises the following amino acid sequence: GVYLLPRRGPRLGVRTTRKSSERSQPRGRRQRIPKAASSQGKAWGKPGYP (SEQ ID NO:242); where the TP has a length of from 40 amino acids to 50 amino acids (e.g., the TP has a length of 40 amino acids (aa), 41 aa, 42 aa, 43 aa, 44 aa, 45 aa, 46 aa, 47 aa, 48 aa, 49 aa, or 50 aa). In some cases, the TP has a length of 50 amino acids. In some cases, the T-cell epitope polypeptide is part of a fusion polypeptide, as described in detail below.

A TP50-C polypeptide encoded by an RNA molecule of the present disclosure can include one or more CD4 epitopes, e.g., one or more of: i) a stretch of about 25 contiguous amino acids having the amino acid sequence: RRGPRLGVRATRKTSERSQPRGRRQ (SEQ ID NO:243) or RRGPRLGVRGTRKSSERSQPRGRRQ (SEQ ID NO:244); ii) a stretch of about 24 contiguous amino acids having the amino acid sequence: RATRKTSERSQPRGRRQPIPKARR (SEQ ID NO:245), or RGTRKSSERSQPRGRRQRIPKAAQ (SEQ ID NO:246), or RATRKTSERSQPRGRRQPIPKARQ (SEQ ID NO:247), or RATRKTSERSQPRGRRQPIPKDRR (SEQ ID NO:248); or iii) a stretch of about 20 contiguous amino acids having the amino acid sequence: RRQPIPKARRSEGRSWAQPG (SEQ ID NO:249), or RRQPIPKARPSEGRTWAQPG (SEQ ID NO:250), or RRQPIPKARRPEGRAWAQPG (SEQ ID NO:251), or RRQPIPKDRRSTGKSWGKPG (SEQ ID NO:252), or RRQPIPKDRRSTGKSWGKPG (SEQ ID NO:252), or RRQPIPKARRPTGRSWGQPG (SEQ ID NO:253), or RRQPIPKARQPTGRHWAQPG (SEQ ID NO:254), or RRQRIPKAASSQGKAWGKPG (SEQ ID NO:255).

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GVYLLPRRGPRLGVRATRKTSERSQPRGRRQPIPKARRSEGRSWAQPGYP (SEQ ID NO:101); and has a length of from 50 amino acids to about 60 amino acids.

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GVYLLPRRGPRLGVRATRKTSERSQPRGRRQPIPKARRSEGRSWAQPGYP (SEQ ID NO:101); and has a length of 50 amino acids. In some cases, a TP encoded by an RNA molecule of the present disclosure comprises the amino acid sequence GVYLLPRRGPRLGVRATRKTSERSQPRGRRQPIPKARRSEGRSWAQPGYP (SEQ ID NO:101); and has a length of 50 amino acids.

### TP23 T-Cell Epitope Polypeptides

In some cases, a TP encoded by an RNA molecule of the present disclosure comprises an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: DVVVVATDALMTGFTGDFDSVID (SEQ ID NO:256; also referred to herein as “TP23”); where the TP has a length of from 18 amino acids to 23 amino acids (e.g., the TP has a length of 18 amino acids (aa), 19 aa, 20 aa, 21 aa, 22 aa, or 23 aa). In some cases, the TP has a length of 23 amino acids. In some cases, the TP is part of a fusion polypeptide, as described in detail below.

In some cases, a TP23 polypeptide encoded by an RNA molecule of the present disclosure comprises the following amino acid sequence: DVVVX₁X₂TDALMTGX₃TGDFDSVID (SEQ ID NO:257), where X₁ is V or C; X₂ is A or S; and X₃ is F or Y; where the TP23 T-cell epitope polypeptide has a length of from 18 amino acids to 23 amino acids (e.g., the TP has a length of 18 amino acids (aa), 19 aa, 20 aa, 21 aa, 22 aa, or 23 aa). In some cases, the TP has a length of 23 amino acids. In some cases, the TP is part of a fusion polypeptide, as described in detail below.

In some cases, a TP23 polypeptide encoded by an RNA molecule of the present disclosure comprises the following amino acid sequence: DVVVVATDALMTGYTGDFDSVID (SEQ ID NO:258); where the TP23 polypeptide has a length of from 18 amino acids to 23 amino acids (e.g., the TP has a length of 18 amino acids (aa), 19 aa, 20 aa, 21 aa, 22 aa, or 23 aa). In some cases, the TP has a length of 23 amino acids. In some cases, the TP is part of a fusion polypeptide, as described in detail below.

In some cases, a TP23 polypeptide encoded by an RNA molecule of the present disclosure comprises the following amino acid sequence: DVVVCATDALMTGFTGDFDSVID (SEQ ID NO:259); where the TP23 T-cell epitope polypeptide has a length of from 18 amino acids to 23 amino acids (e.g., T-cell epitope polypeptide has a length of 18 amino acids (aa), 19 aa, 20 aa, 21 aa, 22 aa, or 23 aa). In some cases, the TP has a length of 23 amino acids. In some cases, the TP is part of a fusion polypeptide, as described in detail below.

In some cases, a TP23 polypeptide encoded by an RNA molecule of the present disclosure comprises the following amino acid sequence: DVVVCSTDALMTGFTGDFDSVID (SEQ ID NO:260); where the TP23 polypeptide has a length of from 18 amino acids to 23 amino acids (e.g., the TP has a length of 18 amino acids (aa), 19 aa, 20 aa, 21 aa, 22 aa, or 23 aa). In some cases, the TP has a length of 23 amino acids. In some cases, the TP is part of a fusion polypeptide, as described in detail below.

In some cases, a TP23 polypeptide encoded by an RNA molecule of the present disclosure comprises the following amino acid sequence: DVVVCATDALMTGYTGDFDSVID (SEQ ID NO:261); where the TP23 T-cell epitope polypeptide has a length of from 18 amino acids to 23 amino acids (e.g., T-cell epitope polypeptide has a length of 18 amino acids (aa), 19 aa, 20 aa, 21 aa, 22 aa, or 23 aa). In some cases, the TP23 T-cell epitope polypeptide has a length of 23 amino acids. In some cases, the TP23 T-cell epitope polypeptide is part of a fusion polypeptide, as described in detail below.

A TP23 polypeptide encoded by an RNA molecule of the present disclosure can include one or more CD8 epitopes, e.g., one or both of: i) a stretch of about 9 contiguous amino acids having the amino acid sequence: ATDALMTGF (SEQ ID NO:262), or STDALMTGF (SEQ ID NO:263), or ATDALMTGY (SEQ ID NO:264); or ii) a stretch of about 9 contiguous amino acids having the amino acid sequence GFTGDFDSV (SEQ ID NO:265), or GYTGDFDSV (SEQ ID NO:266).

A TP23 polypeptide encoded by an RNA molecule of the present disclosure can include one or more CD4 epitopes, e.g., a stretch of about 15 contiguous amino acids having the amino acid sequence: ATDALMTGFTGDFDS (SEQ ID NO:267), or STDALMTGFTGDFDS (SEQ ID NO:268), or ATDALMTGYTGDFDS (SEQ ID NO:269), or ALMTGFTGDFDSVID (SEQ ID NO:270), or ALMTGYTGDFDSVID (SEQ ID NO:271).

In some cases, a TP23 polypeptide encoded by an RNA molecule of the present disclosure includes: a) one or more CD8 epitopes, e.g., one or both of: i) a stretch of about 9 contiguous amino acids having the amino acid sequence: ATDALMTGF (SEQ ID NO:262), or STDALMTGF (SEQ ID NO:263), or ATDALMTGY (SEQ ID NO:264); or ii) a stretch of about 9 contiguous amino acids having the amino acid sequence GFTGDFDSV (SEQ ID NO:265), or GYTGDFDSV (SEQ ID NO:266); and b) one or more CD4 epitopes, e.g., a stretch of about 15 contiguous amino acids having the amino acid sequence: ATDALMTGFTGDFDS (SEQ ID NO:267), or STDALMTGFTGDFDS (SEQ ID NO:268), or ATDALMTGYTGDFDS (SEQ ID NO:269), or ALMTGFTGDFDSVID (SEQ ID NO:270), or ALMTGYTGDFDSVID (SEQ ID NO:271).

In some cases, an RNA molecule of the present disclosure encodes only a TP comprising an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: DVVVVATDALMTGFTGDFDSVID (SEQ ID NO:256; “TP23”); where the T-cell epitope polypeptide has a length of from 23 amino acids to 30 amino acids (e.g., T-cell epitope polypeptide has a length of 23 amino acids (aa), 24 aa, 25 aa, 26 aa, 27 aa, 28 aa, 29 aa, or 30 aa).

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: DVVVVATDALMTGFTGDFDSVID (SEQ ID NO:256); and has a length of 23 amino acids. In some cases, a T-cell epitope polypeptide encoded by an RNA molecule of the present disclosure comprises the amino acid sequence DVVVVATDALMTGFTGDFDSVID (SEQ ID NO:256); and has a length of 23 amino acids.

### TP27 T-Cell Epitope Polypeptides

In some cases, a TP encoded by an RNA molecule of the present disclosure comprises an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: LEQIKGGRHLIFCHSKKKCDELAAKLT (SEQ ID NO:272; also referred to herein as “TP27”); where the TP has a length of from 22 amino acids to 27 amino acids (e.g., the TP has a length of 22 amino acids (aa), 23 aa, 24 aa, 25 aa, 26 aa, or 27 aa). In some cases, the TP is part of a fusion polypeptide, as described in detail below.

In some cases, a TP encoded by an RNA molecule of the present disclosure comprises an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: LEQIKGGRHLIFCHSKKKCDELAAKLR (SEQ ID NO:103); where the TP has a length of from 22 amino acids to 27 amino acids (e.g., the TP has a length of 22 amino acids (aa), 23 aa, 24 aa, 25 aa, 26 aa, or 27 aa). In some cases, the T-cell epitope polypeptide has a length of 27 amino acids. In some cases, the TP is part of a fusion polypeptide, as described in detail below.

In some cases, a TP encoded by an RNA molecule of the present disclosure comprises the following amino acid sequence: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S; where the TP has a length of from 22 amino acids to 27 amino acids (e.g., the TP has a length of 22 amino acids (aa), 23 aa, 24 aa, 25 aa, 26 aa, or 27 aa). In some cases, the TP has a length of 27 amino acids. In some cases, the TP is part of a fusion polypeptide, as described in detail below.

In some cases, a TP27 T-cell epitope encoded by an RNA molecule of the present disclosure comprises the following amino acid sequence: LEVIKGGRHLIFCHSKKKCDELAAKLV (SEQ ID NO:274); where the TP has a length of from 22 amino acids to 27 amino acids (e.g., the TP has a length of 22 amino acids (aa), 23 aa, 24 aa, 25 aa, 26 aa, or 27 aa). In some cases, the TP has a length of 27 amino acids. In some cases, the TP is part of a fusion polypeptide, as described in detail below.

In some cases, a TP27 T-cell epitope polypeptide encoded by an RNA molecule of the present disclosure comprises the following amino acid sequence: IETIKGGRHLIFCHSKKKCDELAAKLS (SEQ ID NO:275); where the TP has a length of from 22 amino acids to 27 amino acids (e.g., the TP has a length of 22 amino acids (aa), 23 aa, 24 aa, 25 aa, 26 aa, or 27 aa). In some cases, the TP has a length of 27 amino acids. In some cases, the TP is part of a fusion polypeptide, as described in detail below.

In some cases, a TP27 T-cell epitope polypeptide encoded by an RNA molecule of the present disclosure comprises the following amino acid sequence: LSYIKGGRHLIFCHSKKKCDELAAALR (SEQ ID NO:276); where the TP has a length of from 22 amino acids to 27 amino acids (e.g., the TP has a length of 22 amino acids (aa), 23 aa, 24 aa, 25 aa, 26 aa, or 27 aa). In some cases, the TP has a length of 27 amino acids. In some cases, the TP is part of a fusion polypeptide, as described in detail below.

In some cases, a TP27 T-cell epitope polypeptide encoded by an RNA molecule of the present disclosure comprises the following amino acid sequence: LAFIKGGRHLIFCHSKKKCDELAALR (SEQ ID NO:277); where the TP has a length of from 22 amino acids to 27 amino acids (e.g., the TP has a length of 22 amino acids (aa), 23 aa, 24 aa, 25 aa, 26 aa, or 27 aa). In some cases, the TP has a length of 27 amino acids. In some cases, the TP is part of a fusion polypeptide, as described in detail below.

In some cases, a TP27 T-cell epitope polypeptide encoded by an RNA molecule of the present disclosure comprises the following amino acid sequence: IAQLKGGRHLIFCHSKKKCDEIASKLR (SEQ ID NO:278); where the TP has a length of from 22 amino acids to 27 amino acids (e.g., the TP has a length of 22 amino acids (aa), 23 aa, 24 aa, 25 aa, 26 aa, or 27 aa). In some cases, the TP has a length of 27 amino acids. In some cases, the TP is part of a fusion polypeptide, as described in detail below.

In some cases, a TP27 T-cell epitope polypeptide encoded by an RNA molecule of the present disclosure comprises the following amino acid sequence: LELIKGGRHLIFCHSKKKCDELAQLT (SEQ ID NO:279); where the TP has a length of from 22 amino acids to 27 amino acids (e.g., the TP has a length of 22 amino acids (aa), 23 aa, 24 aa, 25 aa, 26 aa, or 27 aa). In some cases, the TP has a length of 27 amino acids. In some cases, the TP is part of a fusion polypeptide, as described in detail below.

In some cases, a TP27 T-cell epitope polypeptide encoded by an RNA molecule of the present disclosure comprises the following amino acid sequence: LXLIKGGRHLIFCHSKKKCDELAKQLT (SEQ ID NO:280); where the TP has a length of from 22 amino acids to 27 amino acids (e.g., the TP has a length of 22 amino acids (aa), 23 aa, 24 aa, 25 aa, 26 aa, or 27 aa). In some cases, the TP has a length of 27 amino acids. In some cases, the TP is part of a fusion polypeptide, as described in detail below.

In some cases, a TP27 T-cell epitope polypeptide encoded by an RNA molecule of the present disclosure comprises the following amino acid sequence: LEYIKGGRHLIFCHSKKKCDELAKQLT (SEQ ID NO:281); where the TP has a length of from 22 amino acids to 27 amino acids (e.g., the TP has a length of 22 amino acids (aa), 23 aa, 24 aa, 25 aa, 26 aa, or 27 aa). In some cases, the TP has a length of 27 amino acids. In some cases, the TP is part of a fusion polypeptide, as described in detail below.

In some cases, a TP27 T-cell epitope polypeptide encoded by an RNA molecule of the present disclosure comprises the following amino acid sequence: LQHIKGGRHLIFCHSKKKCDELAGKLT (SEQ ID NO:282); where the TP has a length of from 22 amino acids to 27 amino acids (e.g., the TP has a length of 22 amino acids (aa), 23 aa, 24 aa, 25 aa, 26 aa, or 27 aa). In some cases, the TP has a length of 27 amino acids. In some cases, the TP is part of a fusion polypeptide, as described in detail below.

A TP27 T-cell epitope polypeptide encoded by an RNA molecule of the present disclosure can include one or more CD8 epitopes, e.g., one or both of: i) a stretch of about 9 contiguous amino acids having the amino acid sequence: LIFCHSKKK (SEQ ID NO:283); and ii) a stretch of about 9 contiguous amino acids having the amino acid sequence: HSKKKCDEL (SEQ ID NO:284). In some cases, a TP27 T-cell epitope polypeptide encoded by an RNA molecule of the present disclosure includes the following CD8 epitopes: i) a stretch of about 9 contiguous amino acids having the amino acid sequence: LIFCHSKKK (SEQ ID NO:283); and ii) a stretch of about 9 contiguous amino acids having the amino acid sequence: HSKKKCDEL (SEQ ID NO:284).

A TP27 T-cell epitope polypeptide encoded by an RNA molecule of the present disclosure can include one or more CD4 epitopes, e.g., a stretch of about 15 contiguous amino acids having the amino acid sequence: KGGRHLIFCHSKKKCD (SEQ ID NO:285), or a stretch of about 11 contiguous amino acids having the amino acid sequence: GRHLIFCHSKK (SEQ ID NO:286).

A TP27 T-cell epitope polypeptide encoded by an RNA molecule of the present disclosure can include: a) the following CD8 epitopes: i) a stretch of about 9 contiguous amino acids having the amino acid sequence: LIFCHSKKK (SEQ ID NO:283); and ii) a stretch of about 9 contiguous amino acids having the amino acid sequence: HSKKKCDEL (SEQ ID NO:284); and b) the following CD4 epitope: a stretch of about 11 contiguous amino acids having the amino acid sequence: GRHLIFCHSKK (SEQ ID NO:286). A TP27 T-cell epitope polypeptide encoded by an RNA molecule of the present disclosure can include: a) the following CD8 epitopes: i) a stretch of about 9 contiguous amino acids having the amino acid sequence: LIFCHSKKK (SEQ ID NO:283); and ii) a stretch of about 9 contiguous amino acids having the amino acid sequence: HSKKKCDEL (SEQ ID NO:284); and b) the following CD4 epitope: a stretch of about 15 contiguous amino acids having the amino acid sequence: KGGRHLIFCHSKKKCD (SEQ ID NO:285.

In some cases, an RNA molecule of the present disclosure encodes only a TP comprising an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: LEQIKGGRHLIFCHSKKKCDELAAKLT (SEQ ID NO:272; “TP27”); where the T-cell epitope polypeptide has a length of from 27 amino acids to 34 amino acids (e.g., T-cell epitope polypeptide has a length of 27 amino acids (aa), 28 aa, 29 aa, 30 aa, 31 aa, 32 aa, 33 aa, or 34 aa).

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LEQIKGGRHLIFCHSKKKCDELAAKLT (SEQ ID NO:272); and has a length of 27 amino acids. In some cases, a T-cell epitope polypeptide suitable for inclusion in a composition of the present disclosure comprises the amino acid sequence LEQIKGGRHLIFCHSKKKCDELAAKLT (SEQ ID NO:272); and has a length of 27 amino acids.

### TP35-NS4 T-Cell Epitope Polypeptides

In some cases, a TP encoded by an RNA molecule of the present disclosure comprises an amino acid sequence having at least 20% (e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100%) amino acid sequence identity to the following amino acid sequence: ILRRHVGPGEGAVQWMNRLIAFASRGNHVSPTHYV (SEQ ID NO:287; also referred to herein as “TP35-NS4”); where the TP has a length of from 28 amino acids to 35 amino acids (e.g., the TP has a length of 28 amino acids (aa), 29 aa, 30 aa, 31 aa, 32 aa, 33 aa, 34 aa, or 35 aa). In some cases, the T-cell epitope polypeptide is part of a fusion polypeptide, as described in detail below.

In some cases, a TP35-NS4 T-cell epitope polypeptide encoded by an RNA molecule of the present disclosure comprises the following amino acid sequence: X₁X₂X₃RHX₄GX₅X₆EGX₇X₈QWMNRLIAFASRGNHVX₉PTHYX₁₀ (SEQ ID NO:288), where X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; and X₁₀ is V or I; where the TP35-NS4 T-cell epitope polypeptide has a length of from 28 amino acids to 35 amino acids (e.g., the T-cell epitope polypeptide has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the TP is part of a fusion polypeptide, as described in detail below.

In some cases, a TP35-NS4 T-cell epitope polypeptide encoded by an RNA molecule of the present disclosure comprises the following amino acid sequence: X₁X₂X₃RHX₄GX₅X₆EGX₇X₈QWMNRLIAFASRGNHVX₉PTHYX₁₀ (SEQ ID NO:289), where X₁ is I or V; X₂ is L; X₃ is R; X₄ is V or I; X₅ is P or Q; X₆ is G or A; X₇ is A; X₈ is V or T; X₉ is S or A; and X₁₀ is V; where the TP35-NS4 T-cell epitope polypeptide has a length of from 28 amino acids to 35 amino acids (e.g., the TP has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the TP is part of a fusion polypeptide, as described in detail below.

In some cases, a TP35-NS4 T-cell epitope polypeptide encoded by an RNA molecule of the present disclosure comprises the following amino acid sequence: ILRRHVGPGEGAVQWMNRLIAFASRGNHVAPTHYV (SEQ ID NO:290); where the TP35-NS4 T-cell epitope polypeptide has a length of from 28 amino acids to 35 amino acids (e.g., the TP has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the TP is part of a fusion polypeptide, as described in detail below.

In some cases, a TP35-NS4 T-cell epitope polypeptide encoded by an RNA molecule of the present disclosure comprises the following amino acid sequence: ILRRHVGQGEGAVQWMNRLIAFASRGNHVAPTHYV (SEQ ID NO:291); where the TP35-NS4 T-cell epitope polypeptide has a length of from 28 amino acids to 35 amino acids (e.g., the TP has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the TP is part of a fusion polypeptide, as described in detail below.

In some cases, a TP35-NS4 T-cell epitope polypeptide encoded by an RNA molecule of the present disclosure comprises the following amino acid sequence: VLRRHIGPGEGAVQWMNRLIAFASRGNHVSPTHYV (SEQ ID NO:292); where the TP35-NS4 T-cell epitope polypeptide has a length of from 28 amino acids to 35 amino acids (e.g., the TP has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the TP is part of a fusion polypeptide, as described in detail below.

In some cases, a TP35-NS4 T-cell epitope polypeptide encoded by an RNA molecule of the present disclosure comprises the following amino acid sequence: ILRRHVGPAEGATQWMNRLIAFASRGNHVSPTHYV (SEQ ID NO:293); where the TP35-NS4 T-cell epitope polypeptide has a length of from 28 amino acids to 35 amino acids (e.g., the TP has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the TP is part of a fusion polypeptide, as described in detail below.

In some cases, a TP35-NS4 T-cell epitope polypeptide encoded by an RNA molecule of the present disclosure comprises the following amino acid sequence: IIKRHTGTSEGVTQWMNRLIAFASRGNHVSPTHYI (SEQ ID NO:294); where the TP35-NS4 T-cell epitope polypeptide has a length of from 28 amino acids to 35 amino acids (e.g., the TP has a length of 28 amino acids (aa), 29 aa, 30 aa, 31 aa, 32 aa, 33 aa, 34 aa, or 35 aa). In some cases, the TP35-NS4 T-cell epitope polypeptide has a length of 35 amino acids. In some cases, the TP is part of a fusion polypeptide, as described in detail below.

A TP35-NS4 T-cell epitope polypeptide encoded by an RNA molecule of the present disclosure can include one or more CD8 epitopes, e.g., a stretch of about 16 contiguous amino acids having the amino acid sequence: EGAVQWMNRLIAFASR (SEQ ID NO:295), or EGATQWMNRLIAFASR (SEQ ID NO:296), or EGVTQWMNRLIAFASR (SEQ ID NO:297); or a stretch of about 9 contiguous amino acids having the amino acid sequence: WMNRLIAFA (SEQ ID NO:298).

A TP35-NS4 T-cell epitope polypeptide encoded by an RNA molecule of the present disclosure can include one or more CD4 epitopes, e.g., a stretch of about 15 contiguous amino acids having the amino acid sequence: RHVGPGEGAVQWMNR (SEQ ID NO:299), or RHVGQGEGAVQWMNR (SEQ ID NO:300), or RHIGPGEGAVQWMNR (SEQ ID NO:301), or RHVGPAEGATQWMNR (SEQ ID NO:302), or RHTGTSEGVTQWMNR (SEQ ID NO:303); or a stretch of about 20 contiguous amino acids having the amino acid sequence: HVGPGEGAVQWMNRLIAFAS (SEQ ID NO:304), or HVGQGEGAVQWMNRLIAFAS (SEQ ID NO:305), or HIGPGEGAVQWMNRLIAFAS (SEQ ID NO:306), or HVGPAEGATQWMNRLIAFAS (SEQ ID NO:307), or HTGTSEGVTQWMNRLIAFAS (SEQ ID NO:308); or a stretch of about 20 contiguous amino acids having the amino acid sequence: GAVQWMNRLIAFASRGNHVS (SEQ ID NO:309), or GAVQWMNRLIAFASRGNHVA (SEQ ID NO:310), or GATQWMNRLIAFASRGNHVS (SEQ ID NO:311), or GVTQWMNRLIAFASRGNHVS (SEQ ID NO:312).

In some cases, TP35-NS4 T-cell epitope polypeptide encoded by an RNA molecule of the present disclosure includes: a) one or more CD8 epitopes, e.g., a stretch of about 16 contiguous amino acids having the amino acid sequence: EGAVQWMNRLIAFASR (SEQ ID NO:295), or EGATQWMNRLIAFASR (SEQ ID NO:296), or EGVTQWMNRLIAFASR (SEQ ID NO:297); or a stretch of about 9 contiguous amino acids having the amino acid sequence: WMNRLIAFA (SEQ ID NO:298); and b) one or more CD4 epitopes, e.g., a stretch of about 15 contiguous amino acids having the amino acid sequence: RHVGPGEGAVQWMNR (SEQ ID NO:299), or RHVGQGEGAVQWMNR (SEQ ID NO:300), or RHIGPGEGAVQWMNR (SEQ ID NO:301), or RHVGPAEGATQWMNR (SEQ ID NO:302), or RHTGTSEGVTQWMNR (SEQ ID NO:303); or a stretch of about 20 contiguous amino acids having the amino acid sequence: HVGPGEGAVQWMNRLIAFAS (SEQ ID NO:304), or HVGQGEGAVQWMNRLIAFAS (SEQ ID NO:305), or HIGPGEGAVQWMNRLIAFAS (SEQ ID NO:306), or HVGPAEGATQWMNRLIAFAS (SEQ ID NO:307), or HTGTSEGVTQWMNRLIAFAS (SEQ ID NO:308); or a stretch of about 20 contiguous amino acids having amino acid sequence: GAVQWMNRLIAFASRGNHVS (SEQ ID NO:309), or GAVQWMNRLIAFASRGNHVA (SEQ ID NO:310), or GATQWMNRLIAFASRGNHVS (SEQ ID NO:311), or GVTQWMNRLIAFASRGNHVS (SEQ ID NO:312).

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYV (SEQ ID NO:287); and has a length of from 35 amino acids to 40 amino acids (e.g., 35 amino acids (aa), 36 aa, 37 aa, 38 aa, 39 aa, or 40 aa).

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYV (SEQ ID NO:287); and has a length of 35 amino acids. In some cases, a T-cell epitope polypeptide encoded by an RNA molecule of the present disclosure comprises the amino acid sequence ILRRHVGPGEGAVQWMNRLIAFASRGNHVSPTHYV (SEQ ID NO:287); and has a length of 35 amino acids.

### TP42 T-Cell Epitope Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GTEGEIPFYGKAIPLEQIKGGRHLIFCHSKKKCDELAAKLTG (SEQ ID NO:313); and has a length of from 33 amino acids to about 50 amino acids (e.g., 33 amino acids (aa), 34 aa, 35 aa, 36 aa, 37 aa, 38 aa, 39 aa, 40 aa, 41 aa, 42 aa, or from 42 aa to 50 aa). In some cases, a T-cell epitope polypeptide encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GTEGEIPFYGKAIPLEQIKGGRHLIFCHSKKKCDELAAKLTG (SEQ ID NO:313); and has a length of 42 amino acids. In some cases, a TP encoded by an RNA molecule of the present disclosure comprises the amino acid sequence GTEGEIPFYGKAIPLEQIKGGRHLIFCHSKKKCDELAAKLTG (SEQ ID NO:313); and has a length of 42 amino acids.

In some cases, a TP42 T-cell epitope polypeptide encoded by an RNA molecule of the present disclosure comprises the following amino acid sequence: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:314), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S, wherein n is an integer from 2 to 10, and where the TP42 T-cell epitope polypeptide has a length of from 34 amino acids to 52 amino acids (e.g., 34 amino acids (aa), 35 aa, 36 aa, 37 aa, 38 aa, 39 aa, 40 aa, 41 aa, 42 aa, 43 aa, 44 aa, 45 aa, 46 aa, 47 aa, 48 aa, 49 aa, 50 aa, 51 aa, or 52 aa. In some cases, a TP42 T-cell epitope polypeptide encoded by an RNA molecule of the present disclosure comprises the following amino acid sequence: X₁X₂X₃GEIPFYGX₄AIPX₅X₆X₇X₈KGGRHLIFCHSKKKCDEX₉AX₁₀X₁₁LX₁₂X₁₃KKK (SEQ ID NO:315), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S. In some cases, a TP42 T-cell epitope polypeptide encoded by an RNA molecule of the present disclosure comprises the following amino acid sequence: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:352), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, V, or L; X₈ is I or L; X₉ is L or I; X₁₀ is A, K, or S; X₁₁ is K, Q, or A; X₁₂ is T, R, V, or S; and X₁₃ is G or S, wherein n is an integer from 2 to 10, and where the TP42 T-cell epitope polypeptide has a length of from 34 amino acids to 52 amino acids (e.g., 34 amino acids (aa), 35 aa, 36 aa, 37 aa, 38 aa, 39 aa, 40 aa, 41 aa, 42 aa, 43 aa, 44 aa, 45 aa, 46 aa, 47 aa, 48 aa, 49 aa, 50 aa, 51 aa, or 52 aa. In some cases, a TP42 T-cell epitope polypeptide encoded by an RNA molecule of the present disclosure comprises the following amino acid sequence: X₁X₂X₃GEIPFYGX₄AIPX₅X₆X₇X₈KGGRHLIFCHSKKKCDEX₉AX₁₀X₁₁LX₁₂X₁₃KKK (SEQ ID NO:353), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, V, or L; X₈ is I or L; X₉ is L or I; X₁₀ is A, K, or S; X₁₁ is K, Q, or A; X₁₂ is T, R, V, or S; and X₁₃ is G or S. In some cases, the TP42 T-cell epitope polypeptide has a length of 45 amino acids. In some cases, the TP42 T-cell epitope polypeptide comprises the following amino acid sequence: GTEGEIPFYGKAIPLEQIKGGRHLIFCHSKKKCDELAAKLTGKKK (SEQ ID NO:391) and has a length of 45 amino acids.

In some cases, a TP42 T-cell epitope polypeptide encoded by an RNA molecule of the present disclosure comprises the following amino acid sequence: X₁X₂X₃GEIPFYGX₄AIPX₅X₆X₇X₈KGGRHLIFCHSKKKCDEX₉AX₁₀X₁₁LX₁₂X₁₃ (SEQ ID NO:316), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S; where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the TP has a length of 42 amino acids. In some cases, the TP is part of a fusion polypeptide, as described in detail below.

In some cases, a TP42 T-cell epitope polypeptide encoded by an RNA molecule of the present disclosure comprises the following amino acid sequence: STTGEIPFYGKAIPLEVIKGGRHLIFCHSKKKCDELAAKLVA (SEQ ID NO:317); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the TP has a length of 42 amino acids. In some cases, the TP is part of a fusion polypeptide, as described in detail below.

In some cases, a TP42 T-cell epitope polypeptide encoded by an RNA molecule of the present disclosure comprises the following amino acid sequence: SNTGEIPFYGKAIPIETIKGGRHLIFCHSKKKCDELAAKLSG (SEQ ID NO:318); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the TP has a length of 42 amino acids. In some cases, the TP is part of a fusion polypeptide, as described in detail below.

In some cases, a TP42 T-cell epitope polypeptide encoded by an RNA molecule of the present disclosure comprises the following amino acid sequence: GQEGEIPFYGRAIPLSYIKGGRHLIFCHSKKKCDELAAALRG (SEQ ID NO:319); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the TP has a length of 42 amino acids. In some cases, the TP is part of a fusion polypeptide, as described in detail below.

In some cases, a TP42 T-cell epitope polypeptide encoded by an RNA molecule of the present disclosure comprises the following amino acid sequence: GHEGEIPFYGKAIPLAFIKGGRHLIFCHSKKKCDELAALRG (SEQ ID NO:320); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the TP has a length of 42 amino acids. In some cases, the TP is part of a fusion polypeptide, as described in detail below.

In some cases, a TP42 T-cell epitope polypeptide encoded by an RNA molecule of the present disclosure comprises the following amino acid sequence: GSEGEIPFYGKAIPIAQLKGGRHLIFCHSKKKCDEIASKLRG (SEQ ID NO:321); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the TP has a length of 42 amino acids. In some cases, the TP is part of a fusion polypeptide, as described in detail below.

In some cases, a TP42 T-cell epitope polypeptide encoded by an RNA molecule of the present disclosure comprises the following amino acid sequence: PTTGEIPFYGKAIPLELIKGGRHLIFCHSKKKCDELAQLTS (SEQ ID NO:322); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the TP has a length of 42 amino acids. In some cases, the TP is part of a fusion polypeptide, as described in detail below.

In some cases, a TP42 T-cell epitope polypeptide encoded by an RNA molecule of the present disclosure comprises the following amino acid sequence: PSEGEIPFYGRAIPLXLIKGGRHLIFCHSKKKCDELAKQLTS (SEQ ID NO:323); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the TP has a length of 42 amino acids. In some cases, the TP is part of a fusion polypeptide, as described in detail below.

In some cases, a TP42 T-cell epitope polypeptide encoded by an RNA molecule of the present disclosure comprises the following amino acid sequence: PTTGEIPFYGKAIPLEYIKGGRHLIFCHSKKKCDELAKQLTS (SEQ ID NO:324); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the TP has a length of 42 amino acids. In some cases, the TP is part of a fusion polypeptide, as described in detail below.

In some cases, a TP42 T-cell epitope polypeptide encoded by an RNA molecule of the present disclosure comprises the following amino acid sequence: GNDGEIPFYGKAIPLQHIKGGRHLIFCHSKKKCDELAGKLTS (SEQ ID NO:325); where the TP42 T-cell epitope polypeptide has a length of from 33 amino acids to 42 amino acids (e.g., 33 amino acids (aa), 34 aa, 35 aa, 36 aa, 37 aa, 38 aa, 39 aa, 40 aa, 41 aa, or 42 aa). In some cases, the TP42 T-cell epitope polypeptide has a length of 42 amino acids. In some cases, the TP has a length of 42 amino acids. In some cases, the TP is part of a fusion polypeptide, as described in detail below.

A TP42 T-cell epitope polypeptide encoded by an RNA molecule of the present disclosure can include one or more CD8 epitopes, e.g., one or more of: i) a stretch of about 11 contiguous amino acids having the amino acid sequence: FYGX₁AIPX₂X₃X₄X₅(SEQ ID NO:326), where X₁ is K or R; X₂ is L or I; X₃ is E, S, A, Q, or V; X₄ is Q, T, Y, F, L, or H; and X₅ is I or L; ii) a stretch of about 9 contiguous amino acids having the amino acid sequence: LIFCHSKKK (SEQ ID NO:283); and iii) a stretch of about 9 contiguous amino acids having the amino acid sequence: HSKKKCDEL (SEQ ID NO:284). In some cases, a TP42 T-cell epitope polypeptide encoded by an RNA molecule of the present disclosure includes the following CD8 epitopes: i) a stretch of about 11 contiguous amino acids having the amino acid sequence: FYGKAIPLEQI (SEQ ID NO:327); ii) ii) a stretch of about 9 contiguous amino acids having the amino acid sequence: LIFCHSKKK (SEQ ID NO:283); and iii) a stretch of about 9 contiguous amino acids having the amino acid sequence: HSKKKCDEL (SEQ ID NO:284).

A TP42 T-cell epitope polypeptide encoded by an RNA molecule of the present disclosure can include one or more CD4 epitopes, e.g., a stretch of about 15 contiguous amino acids having the amino acid sequence: KGGRHLIFCHSKKKCD (SEQ ID NO:285), or a stretch of about 11 contiguous amino acids having the amino acid sequence: GRHLIFCHSKK (SEQ ID NO:286).

A TP42 T-cell epitope polypeptide encoded by an RNA molecule of the present disclosure can include: a) the following CD8 epitopes: i) a stretch of about 11 contiguous amino acids having the amino acid sequence: FYGKAIPLEQI (SEQ ID NO:327); ii) ii) a stretch of about 9 contiguous amino acids having the amino acid sequence: LIFCHSKKK (SEQ ID NO:283); and iii) a stretch of about 9 contiguous amino acids having the amino acid sequence: HSKKKCDEL (SEQ ID NO:284); and b) the following CD4 epitope: a stretch of about 11 contiguous amino acids having the amino acid sequence: GRHLIFCHSKK (SEQ ID NO:286). A TP42 T-cell epitope polypeptide encoded by an RNA molecule of the present disclosure can include: a) the following CD8 epitopes: i) a stretch of about 11 contiguous amino acids having the amino acid sequence: FYGKAIPLEQI (SEQ ID NO:327); ii) ii) a stretch of about 9 contiguous amino acids having the amino acid sequence: LIFCHSKKK (SEQ ID NO:283); and iii) a stretch of about 9 contiguous amino acids having the amino acid sequence: HSKKKCDEL (SEQ ID NO:284); and b) the following CD4 epitope: a stretch of about 15 contiguous amino acids having the amino acid sequence: KGGRHLIFCHSKKKCD (SEQ ID NO:285).

In some cases, a suitable TP is a TP42 polypeptide with from 1 amino acid to 5 amino acids removed from the N-terminus and/or from 1 amino acid to 5 amino acids removed from the C-terminus. Thus, e.g., a suitable TP can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: IPFYGKAIPLEQIKGGRHLIFCHSKKKCDELAAKLTG (SEQ ID NO:328); and has a length of 37 amino acids. As another example, a suitable TP can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: GTEGEIPFYGKAIPLEQIKGGRHLIFCHSKKKCDELA (SEQ ID NO:329); and has a length of 37 amino acids.

### TP45 T-Cell Epitope Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LNAVAYYRGLDVSVIPTSGDVVVVATDALMTGFTGDFDSVIDCNV (SEQ ID NO:330); and has a length of from 45 amino acids to about 50 amino acids. In some cases, a T-cell epitope polypeptide encoded by an RNA molecule of the present disclosure an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LNAVAYYRGLDVSVIPTSGDVVVVATDALMTGFTGDFDSVIDCNV (SEQ ID NO:330); and has a length of 45 amino acids. In some cases, a TP encoded by an RNA molecule of the present disclosure comprises the amino acid sequence LNAVAYYRGLDVSVIPTSGDVVVVATDALMTGFTGDFDSVIDCNV (SEQ ID NO:330); and has a length of 45 amino acids. In some cases, a TP encoded by an RNA molecule of the present disclosure comprises the amino acid sequence LNAVAYYRGLDVSVIPTSGDVVVVATDALMTGFTGDFDSVIDKKK (SEQ ID NO:331); and has a length of 45 amino acids.

In some cases, a TP45 T-cell epitope polypeptide encoded by an RNA molecule of the present disclosure comprises the following amino acid sequence: X₁X₂AVAX₃YRGX₄DVX₅X₆IPX₇X₈GDVVVX₉X₁₀TDALMTGX₁₁TGDFDSVIDX₁₂X₁₃X₁₄(K)n (SEQ ID NO:332), where X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K, wherein n is an integer from 2 to 10, and where the TP45 T-cell epitope polypeptide has a length of from 36 amino acids to 55 amino acids. In some cases, a TP45 T-cell epitope polypeptide encoded by an RNA molecule of the present disclosure comprises the following amino acid sequence: X₁X₂AVAX₃YRGX₄DVX₅X₆IPX₇X₈GDVVVX₉X₁₀TDALMTGX₁₁TGDFDSVIDX₁₂X₁₃X₁₄KKK (SEQ ID NO:333), where X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K; where the TP45 T-cell epitope polypeptide has a length of 48 amino acids. In some cases, the TP45 T-cell epitope polypeptide comprises the following amino acid sequence: LNAVAYYRGLDVSVIPTSGDVVVVATDALMTGFTGDFDSVIDCNVKKK (SEQ ID NO:334) and has a length of 48 amino acids.

In some cases, a TP45 T-cell epitope polypeptide encoded by an RNA molecule of the present disclosure comprises the following amino acid sequence: X₁X₂AVAX₃YRGX₄DVX₅X₆IPX₇X₈GDVVVX₉X₁₀TDALMTGX₁₁TGDFDSVIDX₁₂X₁₃X₁₄ (SEQ ID NO:335), where X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₁ is F or Y; X₁₂ is C or K; X₁₃ is N or K; and X₁₄ is V or K; where the TP23 T-cell epitope polypeptide has a length of from 18 amino acids to 23 amino acids (e.g., the TP has a length of 18 amino acids (aa), 19 aa, 20 aa, 21 aa, 22 aa, or 23 aa). In some cases, the TP23 T-cell epitope polypeptide has a length of 23 amino acids. In some cases, the TP23 T-cell epitope polypeptide is part of a fusion polypeptide, as described in detail below.

In some cases, a TP45 T-cell epitope polypeptide encoded by an RNA molecule of the present disclosure comprises the following amino acid sequence: X₁X₂AVAX₃YRGX₄DVX₅X₆IPX₇X₈GDVVVX₉X₁₀TDALMTGX₁₁TGDFDSVIDX₁₂X₁₃X₁₄ (SEQ ID NO:336), where X₁ is L; X₂ is N; X₃ is Y; X₄ is L; X₅ is S; X₆ is V; X₇ is T; X₈ is S, Q, or T; X₉ is V or C; X₁₀ is A; X₁₁ is F or Y; X₁₂ is K; X₁₃ is K; and X₁₄ is K; where the TP23 T-cell epitope polypeptide has a length of from 18 amino acids to 23 amino acids (e.g., T-cell epitope polypeptide has a length of 18 amino acids (aa), 19 aa, 20 aa, 21 aa, 22 aa, or 23 aa). In some cases, the TP23 T-cell epitope polypeptide has a length of 23 amino acids. In some cases, the TP23 T-cell epitope polypeptide is part of a fusion polypeptide, as described in detail below.

In some cases, a suitable T-cell epitope polypeptide is a TP45 polypeptide with from 1 amino acid to 5 amino acids removed from the N-terminus and/or from 1 amino acid to 5 amino acids removed from the C-terminus. Thus, for example, in some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LNAVAYYRGLDVSVIPTSGDVVVVATDALMTGFTGDFDSVID (SEQ ID NO:337); and has a length of 43 amino acids. For example, in some cases, a TP encoded by an RNA molecule of the present disclosure comprises the amino acid sequence LNAVAYYRGLDVSVIPTSGDVVVVATDALMTGFTGDFDSVID (SEQ ID NO:337); and has a length of 43 amino acids.

### TP48 T-Cell Epitope Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYVPESDASARVTQIL (SEQ ID NO:338); and has a length of from 48 amino acids to about 55 amino acids, or has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYVPESDASARVTQIL (SEQ ID NO:338); and has a length of 48 amino acids. In some cases, a TP encoded by an RNA molecule of the present disclosure comprises the amino acid sequence ILRRHVGPGEGAVQWMNRLIAFASRGNHVSPTHYVPESDASARVTQIL (SEQ ID NO:338); and has a length of 48 amino acids.

In some cases, a TP48 T-cell epitope encoded by an RNA molecule of the present disclosure comprises the following amino acid sequence: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, where n is an integer from 2 to 10, and where the TP48 T-cell epitope polypeptide has a length of from 38 amino acids to 58 amino acids. In some cases, a TP48 T-cell epitope polypeptide encoded by an RNA molecule of the present disclosure comprises the following amino acid sequence: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉LKKK (SEQ ID NO:340), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, where the TP48 T-cell epitope polypeptide has a length of 51 amino acids. In some cases, the TP48 T-cell epitope polypeptide comprises the amino acid sequence: ILRRHVGPGEGAVQWMNRLIAFASRGNHVSPTHYVPESDASARVTQILKKK (SEQ ID NO:341) and has a length of 51 amino acids.

In some cases, a TP48 T-cell epitope polypeptide encoded by an RNA molecule of the present disclosure comprises the following amino acid sequence: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L (SEQ ID NO:342), where X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V; where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the T-cell epitope polypeptide is part of a fusion polypeptide, as described in detail below.

In some cases, a TP48 T-cell epitope polypeptide encoded by an RNA molecule of the present disclosure comprises the following amino acid sequence: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L (SEQ ID NO:343), where X₁ is I or V; X₂ is L; X₃ is R; X₄ is V or I; X₅ is P or Q; X₆ is G or A; X₇ is A; X₈ is V or T; X₉ is S or A; X₁₀ is V; X₁₁ is P, T, or A; X₁₂ is E; X₁₃ is S or T; X₁₄ is S or A; X₁₅ is A, Q, or R; X₁₆ is R or K; X₁₇ is T; X₁₈ is Q, A, or T; and X₁₉ is I, L, or V; where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the TP is part of a fusion polypeptide, as described in detail below.

In some cases, a TP48 T-cell epitope polypeptide encoded by an RNA molecule of the present disclosure comprises the following amino acid sequence: ILRRHVGPGEGAVQWMNRLIAFASRGNHVSPTHYVPESDAAARVTAIL (SEQ ID NO:344); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the TP is part of a fusion polypeptide, as described in detail below.

In some cases, a TP48 T-cell epitope polypeptide encoded by an RNA molecule of the present disclosure comprises the following amino acid sequence: ILRRHVGPGEGAVQWMNRLIAFASRGNHVSPTHYVPESDAAARVTQIL (SEQ ID NO:104); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the TP is part of a fusion polypeptide, as described in detail below.

In some cases, a TP48 T-cell epitope polypeptide encoded by an RNA molecule of the present disclosure comprises the following amino acid sequence: ILRRHVGPGEGAVQWMNRLIAFASRGNHVAPTHYVTESDASQRVTQLL (SEQ ID NO:346); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the TP is part of a fusion polypeptide, as described in detail below.

In some cases, a TP48 T-cell epitope polypeptide encoded by an RNA molecule of the present disclosure comprises the following amino acid sequence: ILRRHVGQGEGAVQWMNRLIAFASRGNHVAPTHYVAESDASQRVTQVL (SEQ ID NO:347); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the T-cell epitope polypeptide is part of a fusion polypeptide, as described in detail below.

In some cases, a TP48 T-cell epitope polypeptide encoded by an RNA molecule of the present disclosure comprises the following amino acid sequence: ILRRHVGPGEGAVQWMNRLIAFASRGNHVSPTHYVPESDAAARVTALL (SEQ ID NO:348); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the TP is part of a fusion polypeptide, as described in detail below.

In some cases, a TP48 T-cell epitope encoded by an RNA molecule of the present disclosure comprises the following amino acid sequence: ILRRHVGPGEGAVQWMNRLIAFASRGNHVSPTHYVPESDAAARVTQIL (SEQ ID NO:104); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the TP is part of a fusion polypeptide, as described in detail below.

In some cases, a TP48 T-cell epitope polypeptide encoded by an RNA molecule of the present disclosure comprises the following amino acid sequence: VLRRHIGPGEGAVQWMNRLIAFASRGNHVSPTHYV PETDASAKVTQLL (SEQ ID NO:349); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the TP is part of a fusion polypeptide, as described in detail below.

In some cases, a TP48 T-cell epitope polypeptide encoded by an RNA molecule of the present disclosure comprises the following amino acid sequence: ILRRHVGPAEGATQWMNRLIAFASRGNHVSPTHYVPETDASRXVTTIL (SEQ ID NO:350); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the TP is part of a fusion polypeptide, as described in detail below.

In some cases, a TP48 T-cell epitope polypeptide encoded by an RNA molecule of the present disclosure comprises the following amino acid sequence: IIKRHTGTSEGVTQWMNRLIAFASRGNHVSPTHYIQDDDASKRVMGIL (SEQ ID NO:351); where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the TP is part of a fusion polypeptide, as described in detail below.

In some cases, a TP48 T-cell epitope polypeptide encoded by an RNA molecule of the present disclosure comprises the following amino acid sequence: ILRRHVGPAEGATQWMNRLIAFASRGNHVSPTHYVPETDASRXVTTIL (SEQ ID NO:350), where X is R or K; where the TP48 T-cell epitope polypeptide has a length of from 40 amino acids to about 48 amino acids (e.g., has a length of 40 amino acids (aa), 41 aa, 42 aa, 43 aa, 44 aa, 45 aa, 46 aa, 47 aa, or 48 aa). In some cases, the TP48 T-cell epitope polypeptide has a length of 48 amino acids. In some cases, the TP is part of a fusion polypeptide, as described in detail below.

A TP48 T-cell epitope polypeptide encoded by an RNA molecule of the present disclosure can include one or more CD8 epitopes, e.g., a stretch of about 16 contiguous amino acids having the amino acid sequence: EGAVQWMNRLIAFASR (SEQ ID NO:295), or EGATQWMNRLIAFASR (SEQ ID NO:296), or EGVTQWMNRLIAFASR (SEQ ID NO:297); or a stretch of about 9 contiguous amino acids having the amino acid sequence: WMNRLIAFA (SEQ ID NO:298); or a stretch of about 15 contiguous amino acids having the amino acid sequence: PTHYX₁X₂X₃X₄DAX₅X₆X₇VX₈ (SEQ ID NO:354), where X₁ is V or I; X₂ is P, T, A, or Q, X₃ is E or D; X₄ is S, T, or D; X₅ is S or A; X₆ is A, Q, R, or K; X₇ is R, K, or X; and X₈ is T or M.

A TP48 T-cell epitope polypeptide encoded by an RNA molecule of the present disclosure can include one or more CD8 epitopes, e.g., a stretch of about 16 contiguous amino acids having the amino acid sequence: EGAVQWMNRLIAFASR (SEQ ID NO:295), or EGATQWMNRLIAFASR (SEQ ID NO:296), or EGVTQWMNRLIAFASR (SEQ ID NO:297); or a stretch of about 9 contiguous amino acids having the amino acid sequence: WMNRLIAFA (SEQ ID NO:298); or a stretch of about 15 contiguous amino acids having the amino acid sequence: PTHYX₁X₂X₃X₄DAX₅X₆X₇VX₈ (SEQ ID NO:355), where X₁ is V; X₂ is P, T, or A, X₃ is E; X₄ is S or T; X₅ is S or A; X₆ is A, Q, or R; X₇ is R or K; and X₈ is T.

A TP48 T-cell epitope polypeptide encoded by an RNA molecule of the present disclosure can include one or more CD4 epitopes, e.g., a stretch of about 15 contiguous amino acids having the amino acid sequence: RHVGPGEGAVQWMNR (SEQ ID NO:299), or RHVGQGEGAVQWMNR (SEQ ID NO:300), or RHIGPGEGAVQWMNR (SEQ ID NO:301), or RHVGPAEGATQWMNR (SEQ ID NO:302), or RHTGTSEGVTQWMNR (SEQ ID NO:303); or a stretch of about 20 contiguous amino acids having the amino acid sequence: HVGPGEGAVQWMNRLIAFAS (SEQ ID NO:304), or HVGQGEGAVQWMNRLIAFAS (SEQ ID NO:305), or HIGPGEGAVQWMNRLIAFAS (SEQ ID NO:306), or HVGPAEGATQWMNRLIAFAS (SEQ ID NO:307), or HTGTSEGVTQWMNRLIAFAS (SEQ ID NO:308); or a stretch of about 20 contiguous amino acids having amino acid sequence: GAVQWMNRLIAFASRGNHVS (SEQ ID NO:309), or GAVQWMNRLIAFASRGNHVA (SEQ ID NO:310), or GATQWMNRLIAFASRGNHVS (SEQ ID NO:311), or GVTQWMNRLIAFASRGNHVS (SEQ ID NO:312).

In some cases, TP48 T-cell epitope polypeptide encoded by an RNA molecule of the present disclosure includes: a) one or more CD8 epitopes, e.g., a stretch of about 16 contiguous amino acids having the amino acid sequence: EGAVQWMNRLIAFASR (SEQ ID NO:295), or EGATQWMNRLIAFASR (SEQ ID NO:296), or EGVTQWMNRLIAFASR (SEQ ID NO:297); or a stretch of about 9 contiguous amino acids having the amino acid sequence: WMNRLIAFA (SEQ ID NO:298); or a stretch of about 15 contiguous amino acids having the amino acid sequence: PTHYX₁X₂X₃X₄DAX₅X₆X₇VX₈ (SEQ ID NO:354), where X₁ is V or I; X₂ is P, T, A, or Q, X₃ is E or D; X₄ is S, T, or D; X₅ is S or A; X₆ is A, Q, R, or K; X₇ is R, K, or X; and X₈ is T or M; or a stretch of about 15 contiguous amino acids having the amino acid sequence: PTHYX₁X₂X₃X₄DAX₅X₆X₇VX₈ (SEQ ID NO:355), where X₁ is V; X₂ is P, T, or A, X₃ is E; X₄ is S or T; X₅ is S or A; X₆ is A, Q, or R; X₇ is R or K; and X₈ is T; and b) one or more CD4 epitopes, e.g., a stretch of about 15 contiguous amino acids having the amino acid sequence: RHVGPGEGAVQWMNR (SEQ ID NO:299), or RHVGQGEGAVQWMNR (SEQ ID NO:300), or RHIGPGEGAVQWMNR (SEQ ID NO:301), or RHVGPAEGATQWMNR (SEQ ID NO:302), or RHTGTSEGVTQWMNR (SEQ ID NO:303); or a stretch of about 20 contiguous amino acids having the amino acid sequence: HVGPGEGAVQWMNRLIAFAS (SEQ ID NO:304), or HVGQGEGAVQWMNRLIAFAS (SEQ ID NO:305), or HIGPGEGAVQWMNRLIAFAS (SEQ ID NO:306), or HVGPAEGATQWMNRLIAFAS (SEQ ID NO:307), or HTGTSEGVTQWMNRLIAFAS (SEQ ID NO:308); or a stretch of about 20 contiguous amino acids having the amino acid sequence: GAVQWMNRLIAFASRGNHVS (SEQ ID NO:309), or GAVQWMNRLIAFASRGNHVA (SEQ ID NO:310), or GATQWMNRLIAFASRGNHVS (SEQ ID NO:311), or GVTQWMNRLIAFASRGNHVS (SEQ ID NO:312).

In some cases, a suitable TP is a TP48 polypeptide with from 1 amino acid to 5 amino acids removed from the N-terminus and/or from 1 amino acid to 5 amino acids removed from the C-terminus. Thus, e.g., a suitable TP can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: RHVGPGEGAVQWMNRLIAFASRGNHVSPTHYVPESDASARVTQIL (SEQ ID NO:356); and has a length of 45 amino acids. As another example, a suitable TP can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: ILRRHVGPGEGAVQWMNRLIAFASRGNHVSPTHYVPESDASARVT (SEQ ID NO:357); and has a length of 45 amino acids.

### TP33 T-Cell Epitope Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: HSKKKCDELAAKLTGLGLNAVAYYRGLDVSVIP (SEQ ID NO:358); and has a length of from 33 amino acids to about 37 amino acids (e.g., has a length of 33 amino acids (aa), 34 aa, 35 aa, 36 aa, or 37 aa), or has a length of from 30 amino acids to about 33 amino acids (e.g., has a length of 30 amino acids (aa), 31 aa, 32 aa, or 33 aa). In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: HSKKKCDELAAKLTGLGLNAVAYYRGLDVSVIP (SEQ ID NO:358); and has a length of 33 amino acids. In some cases, a TP encoded by an RNA molecule of the present disclosure comprises the amino acid sequence HSKKKCDELAAKLTGLGLNAVAYYRGLDVSVIP (SEQ ID NO:358); and has a length of 33 amino acids. Such TPs are also referred to herein as “TP33” polypeptides. In some cases, a TP33 T-cell epitope polypeptide comprises a CD8 epitope comprising a contiguous stretch of 19 amino acids of one of the following amino acid sequences: i) DELAAKLTGLGLNAVAYYR (SEQ ID NO:359); ii) DELAAKLVALGINAVAYYR (SEQ ID NO:360); iii) DELAAKLSGLGLNAVAYYR (SEQ ID NO:361); iv) DELAAALRGMGLNAVAYYR (SEQ ID NO:362); v) DELAAALRGMGVNAVAYYR (SEQ ID NO:363); vi) DELASKLRGMGLNAVAYYR (SEQ ID NO:364); vii) DELAAKLRGMGLNAVAYYR (SEQ ID NO:365).

In some cases, a TP33 T-cell epitope polypeptide encoded by an RNA molecule of the present disclosure comprises the following amino acid sequence: HSKKKCDELAX₁X₂LX₃X₄X₅GX₆NAVAYYRGLDVSX₇IP (SEQ ID NO:366), where X₁ is A or S; X₂ is K or A; X₃ is V, S, R, or T; X₄ is A or G; X₅ is L or M; X₆ is I, L, or V; and X₇ is V or I; and has a length of from 33 amino acids to about 37 amino acids (e.g., has a length of 33 amino acids (aa), 34 aa, 35 aa, 36 aa, or 37 aa), or has a length of from 30 amino acids to about 33 amino acids (e.g., has a length of 30 amino acids (aa), 31 aa, 32 aa, or 33 aa). In some cases, a TP33 T-cell epitope polypeptide encoded by an RNA molecule of the present disclosure comprises the following amino acid sequence: HSKKKCDELAX₁X₂LX₃X₄X₅GX₆NAVAYYRGLDVSX₇IP (SEQ ID NO:366), where X₁ is A or S; X₂ is K or A; X₃ is V, S, R, or T; X₄ is A or G; X₅ is L or M; X₆ is I, L, or V; and X₇ is V or I; where the TP33 T-cell epitope polypeptide has a length of 33 amino acids.

In some cases, a TP33 T-cell epitope polypeptide encoded by an RNA molecule of the present disclosure comprises the following amino acid sequence: HSKKKCDELAAKLX₁X₂X₃GX₄NAVAYYRGLDVSVIP (SEQ ID NO:367), where X₁ is V, S, R, or T; X₂ is A or G; X₃ is L or M; and X₄ is I, L, or V; and has a length of from 33 amino acids to about 37 amino acids (e.g., has a length of 33 amino acids (aa), 34 aa, 35 aa, 36 aa, or 37 aa), or has a length of from 30 amino acids to about 33 amino acids (e.g., has a length of 30 amino acids (aa), 31 aa, 32 aa, or 33 aa). In some cases, a TP33 T-cell epitope polypeptide encoded by an RNA molecule of the present disclosure comprises the following amino acid sequence: HSKKKCDELAAKLX₁X₂X₃GX₄NAVAYYRGLDVSVIP (SEQ ID NO:367), where X₁ is V, S, R, or T; X₂ is A or G; X₃ is L or M; and X₄ is I, L, or V; where the TP33 T-cell epitope polypeptide has a length of 33 amino acids.

In some cases, a TP33 T-cell epitope polypeptide encoded by an RNA molecule of the present disclosure comprises the following amino acid sequence: HSKKKCDELAAALRGMGXiNAVAYYRGLDVSX₂IP (SEQ ID NO:368), where X₁ is I, L, or V; and X₂ is V or I; and has a length of from 33 amino acids to about 37 amino acids (e.g., has a length of 33 amino acids (aa), 34 aa, 35 aa, 36 aa, or 37 aa), or has a length of from 30 amino acids to about 33 amino acids (e.g., has a length of 30 amino acids (aa), 31 aa, 32 aa, or 33 aa). In some cases, a TP33 T-cell epitope polypeptide encoded by an RNA molecule of the present disclosure comprises the following amino acid sequence: HSKKKCDELAAALRGMGXiNAVAYYRGLDVSX₂IP (SEQ ID NO:368), where X₁ is I, L, or V; and X₂ is V or I; where the TP33 T-cell epitope polypeptide has a length of 33 amino acids.

In some cases, a TP by an RNA molecule of the present disclosure comprises the amino acid sequence HSKKKCDELAAKLRGMGLNAVAYYRGLDVSVIP (SEQ ID NO:369); and has a length of 33 amino acids.

### TP42-2 T-Cell Epitope Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: KGGRHLIFCHSKKKCDELAAKLTGLGLNAVAYYRGLDVSVIP (SEQ ID NO:370); and has a length of from 42 amino acids to about 46 amino acids (e.g., has a length of 42 amino acids (aa), 43 aa, 44 aa, 45 aa, or 46 aa), or has a length of from 38 amino acids to about 42 amino acids (e.g., has a length of 38 amino acids (aa), 39 aa, 40 aa, 41 aa, or 42 aa). In some cases, a T-cell epitope polypeptide encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: KGGRHLIFCHSKKKCDELAAKLTGLGLNAVAYYRGLDVSVIP (SEQ ID NO:370); and has a length of 42 amino acids. In some cases, a TP encoded by an RNA molecule of the present disclosure comprises the amino acid sequence KGGRHLIFCHSKKKCDELAAKLTGLGLNAVAYYRGLDVSVIP (SEQ ID NO:370); and has a length of 42 amino acids. Such TPs are also referred to herein as “TP42-2” polypeptides. In some cases, a TP42-2 T-cell epitope polypeptide comprises a CD8 epitope comprising a contiguous stretch of 19 amino acids of one of the following amino acid sequences: i) DELAAKLTGLGLNAVAYYR (SEQ ID NO:359); ii) DELAAKLVALGINAVAYYR (SEQ ID NO:360); iii) DELAAKLSGLGLNAVAYYR (SEQ ID NO:361); iv) DELAAALRGMGLNAVAYYR (SEQ ID NO:362); v) DELAAALRGMGVNAVAYYR (SEQ ID NO:363); vi) DELASKLRGMGLNAVAYYR (SEQ ID NO:364); vii) DELAAKLRGMGLNAVAYYR (SEQ ID NO:365).

In some cases, a TP42-2 T-cell epitope polypeptide encoded by an RNA molecule of the present disclosure comprises the following amino acid sequence: KGGRHLIFCHSKKKCDELAX₁X₂LX₃X₄X₅GX₆NAVAYYRGLDVSX₇IP (SEQ ID NO:371), where X₁ is A or S; X₂ is K or A; X₃ is V, S, R, or T; X₄ is A or G; X₅ is L or M; X₆ is I, L, or V; and X₇ is V or I; and has a length of from 42 amino acids to about 46 amino acids (e.g., has a length of 42 amino acids (aa), 43 aa, 44 aa, 45 aa, or 46 aa), or has a length of from 38 amino acids to about 42 amino acids (e.g., has a length of 38 amino acids (aa), 39 aa, 40 aa, 41 aa, or 42 aa). In some cases, a TP42-2 T-cell epitope polypeptide encoded by an RNA molecule of the present disclosure comprises the following amino acid sequence: KGGRHLIFCHSKKKCDELAX₁X₂LX₃X₄X₅GX₆NAVAYYRGLDVSX₇IP (SEQ ID NO:371), where X₁ is A or S; X₂ is K or A; X₃ is V, S, R, or T; X₄ is A or G; X₅ is L or M; X₆ is I, L, or V; and X₇ is V or I; where the TP42-2 T-cell epitope polypeptide has a length of 42 amino acids.

In some cases, a TP42-2 T-cell epitope polypeptide encoded by an RNA molecule of the present disclosure comprises the following amino acid sequence: KGGRHLIFCHSKKKCDELAAKLX₁X₂X₃GX₄NAVAYYRGLDVSVIP (SEQ ID NO:372), where X₁ is V, S, R, or T; X₂ is A or G; X₃ is L or M; and X₄ is I, L, or V; and has a length of from 42 amino acids to about 46 amino acids (e.g., has a length of 42 amino acids (aa), 43 aa, 44 aa, 45 aa, or 46 aa), or has a length of from 38 amino acids to about 42 amino acids (e.g., has a length of 38 amino acids (aa), 39 aa, 40 aa, 41 aa, or 42 aa). In some cases, a TP42-2 T-cell epitope polypeptide encoded by an RNA molecule of the present disclosure comprises the following amino acid sequence: KGGRHLIFCHSKKKCDELAAKLX₁X₂X₃GX₄NAVAYYRGLDVSVIP (SEQ ID NO:372), where X₁ is V, S, R, or T; X₂ is A or G; X₃ is L or M; and X₄ is I, L, or V; where the TP42-2 T-cell epitope polypeptide has a length of 42 amino acids.

In some cases, a TP42-2 T-cell epitope polypeptide encoded by an RNA molecule of the present disclosure comprises the following amino acid sequence: KGGRHLIFCHSKKKCDELAAALRGMGX₁NAVAYYRGLDVSX₂IP (SEQ ID NO:373), where X₁ is I, L, or V; and X₂ is V or I; and has a length of from 42 amino acids to about 46 amino acids (e.g., has a length of 42 amino acids (aa), 43 aa, 44 aa, 45 aa, or 46 aa), or has a length of from 38 amino acids to about 42 amino acids (e.g., has a length of 38 amino acids (aa), 39 aa, 40 aa, 41 aa, or 42 aa). In some cases, a TP42-2 T-cell epitope polypeptide encoded by an RNA molecule of the present disclosure comprises the following amino acid sequence: HSKKKCDELAAALRGMGXiNAVAYYRGLDVSX₁IP (SEQ ID NO:368), where X₁ is I, L, or V; and X₂ is V or I; where the TP42-2 T-cell epitope polypeptide has a length of 42 amino acids.

In some cases, a TP42-2 T-cell epitope polypeptide encoded by an RNA molecule of the present disclosure comprises the amino acid sequence KGGRHLIFCHSKKKCDELAAKLRGMGLNAVAYYRGLDVSVIP (SEQ ID NO:374); and has a length of 42 amino acids.

### TP65 Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: TPIDTTIMAKNEVFCVDPX₆KGGRKPARLIVYPDLGVRVCEKMALYDVVQKLPQAVMGX₇SYGF QYS (SEQ ID NO:375), where X₆ is E, T, or V, and X₇ is S, A, or P; and has a length of from 65 amino acids to 70 amino acids (e.g., 65, 66, 67, 68, 69, or 70 amino acids). In some cases, the TP has a length of 65 amino acids. In some cases, a TP65 polypeptide has the following amino acid sequence: TPIDTTIMAKNEVFCVDPEKGGRKPARLIVYPDLGVRVCEKMALYDVVQKLPQAVMGSSYGFQ YS (SEQ ID NO:376) and has a length of 65 amino acids. In some cases, a TP65 polypeptide has the following amino acid sequence: TPIDTTIMAKNEVFCVDPTKGGRKPARLIVYPDLGVRVCEKMALYDVVQKLPQAVMGASYGFQ YS (SEQ ID NO:377) and has a length of 65 amino acids. In some cases, a TP65 polypeptide has the following amino acid sequence: TPIDTTIMAKNEVFCVDPVKGGRKPARLIVYPDLGVRVCEKMALYDVVQKLPQAVMGPSYGFQ YS (SEQ ID NO:378).

### TP240 Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: SYQVGYLHAPTGSGKSTKVPAAYAAQGYKVLVLNPSVAATLGFGAYMSKAHGIDPNIRTGVRT VTTGAPITYSTYGKFLADGGCSGGAYDIIICDECHSXiDATTILGIGTVLDQAETAGARLVVLATA TPPGSVTVPHPNIEEVALGX₂EGEIPFYGKAIPLX₃X₄IKGGRHLIFCHSKKKCDELAAKLRGMGLN AVAYYRGLDVSVIPTX₅GDVVVVATDALMTGYTGDFDSVIDCNVAVTQT (SEQ ID NO:379), where X₁ is T, V, or Q, X₂ is T, N, Q, H, or S, X₃ is E, S, or A, X₄ is V, T, Y, F, or L, and X₅ is S, Q, or T; and has a length of from 240 amino acids to 245 amino acids (e.g., 240, 241, 242, 243, 244, or 245 amino acids). In some cases, the TP has a length of 240 amino acids.

In some cases, a TP240 polypeptide has the following amino acid sequence: SYQVGYLHAPTGSGKSTKVPAAYAAQGYKVLVLNPSVAATLGFGAYMSKAHGIDPNIRTGVRT VTTGAPITYSTYGKFLADGGCSGGAYDIIICDECHSTDATTILGIGTVLDQAETAGARLVVLATA TPPGSVTVPHPNIEEVALGTEGEIPFYGKAIPLEVIKGGRHLIFCHSKKKCDELAAKLRGMGLNA VAYYRGLDVSVIPTSGDVVVVATDALMTGYTGDFDSVIDCNVAVTQT (SEQ ID NO:380), and has a length of 240 amino acids. In some cases, a TP240 polypeptide has the following amino acid sequence: SYQVGYLHAPTGSGKSTKVPAAYAAQGYKVLVLNPSVAATLGFGAYMSKAHGIDPNIRTGVRT VTTGAPITYSTYGKFLADGGCSGGAYDIIICDECHSVDATTILGIGTVLDQAETAGARLVVLATA TPPGSVTVPHPNIEEVALGNEGEIPFYGKAIPLSTIKGGRHLIFCHSKKKCDELAAKLRGMGLNA VAYYRGLDVSVIPTQGDVVVVATDALMTGYTGDFDSVIDCNVAVTQT (SEQ ID NO:381), and has a length of 240 amino acids.

### TP29 T-Cell Epitope Polypeptides

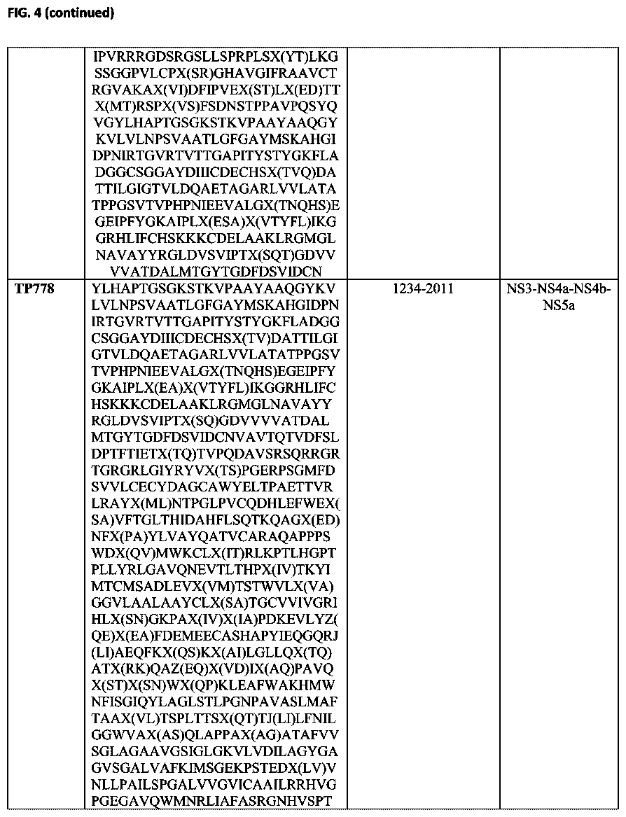

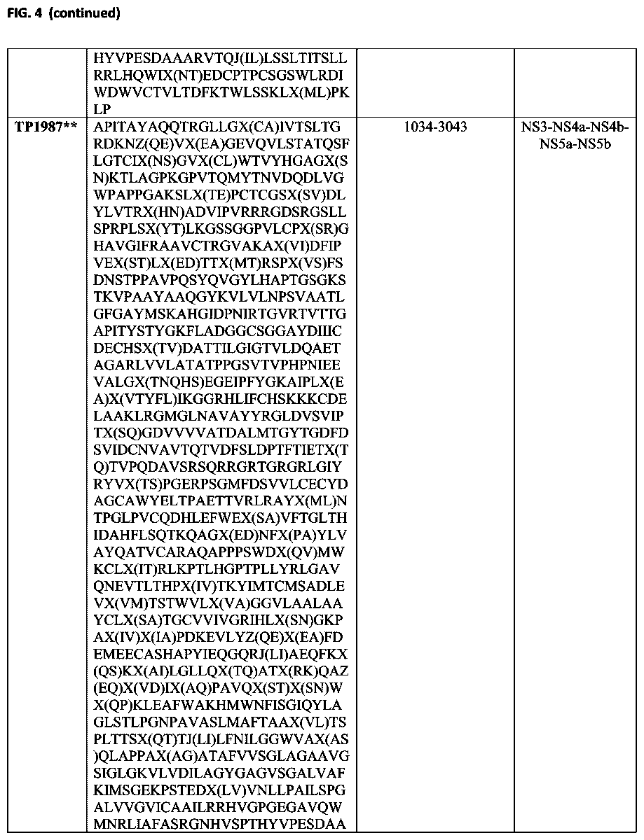

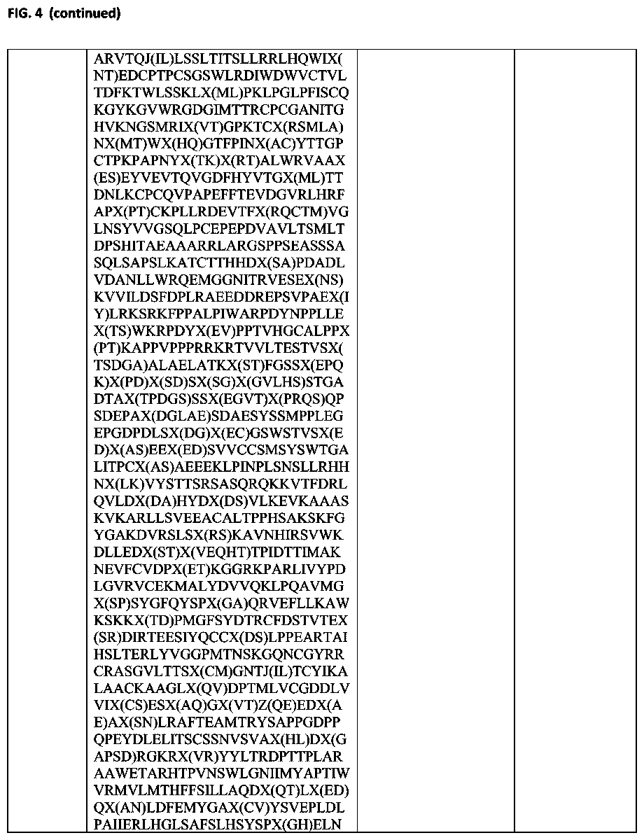

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP29 amino acid sequence depicted in FIG. 4; and has a length of about 29 amino acids. For example, in some cases, a TP29 polypeptide comprises the amino acid sequence: AIPLX(EA)X(VTYFL)IKGGRHLIFCHSKKKCDELAAKL (SEQ ID NO:52), where the first “X” is Glu or Ala, and the second “X” is Val, Thr, Tyr, Phe, or Leu; and where the TP29 polypeptide has a length of 29 amino acids.

### TP50-NS2 T-Cell Epitope Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP50-NS2 amino acid sequence depicted in FIG. 4; and has a length of about 50 amino acids. For example, in some cases, a TP50-NS2 polypeptide comprises the amino acid sequence: LGRWTGTYX(VI)YDHLTPLX(RS)DWAAAGLRDLAVAVEPVVFSPMEX(TK)KVITWGADT (SEQ ID NO:53), where the first “X” is Val or Ile, the second “X” is Arg or Ser, and the third “X” is Thr or Lys; and where the TP50-NS2 polypeptide has a length of 50 amino acids.

### TP52 T-Cell Epitope Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP52 amino acid sequence depicted in FIG. 4; and has a length of about 52 amino acids. For example, in some cases, a TP52 polypeptide comprises the amino acid sequence: AIPLX(EA)X(VTYFL)IKGGRHLIFCHSKKKCDELAAKLRGMGLNAVAYYRGLDVSVIPT X(SQ) (SEQ ID NO:54), where the first “X” is Glu or Ala, the second “X” is Val, Thr, Tyr, Phe, or Leu, and the third “X” is Ser or Gln; and where the TP52 polypeptide has a length of 52 amino acids.

### TP70 T-Cell Epitope Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP70 amino acid sequence depicted in FIG. 4; and has a length of about 70 amino acids. For example, in some cases, a TP70 polypeptide comprises the TP70 amino acid sequence depicted in FIG. 4, where X is Ser or Gln; and where the TP70 polypeptide has a length of 70 amino acids.

### TP100 T-Cell Epitope Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP100 amino acid sequence depicted in FIG. 4; and has a length of about 100 amino acids. For example, in some cases, a TP100 polypeptide comprises the TP100 amino acid sequence depicted in FIG. 4, where the first “X” is Thr, Asn, Gln, His, or Ser, where the second “X” is Glue or Ala, the third “X” is Val, Thr, Tyr, Phe, or Leu, and the fourth “X” is Ser or Gln; and where the TP100 polypeptide has a length of 100 amino acids.

### TP171 T-Cell Epitope Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP171 amino acid sequence depicted in FIG. 4; and has a length of about 171 amino acids.

### TP228 T-Cell Epitope Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP228 amino acid sequence depicted in FIG. 4; and has a length of about 228 amino acids.

A TP228 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following TP228 amino acid sequence: LHAPTGSGKSTKVPAAYAAQGYKVLVLNPSVAATLGFGAYMSKAHGIDPNIRTGVRTVTTGAP ITYSTYGKFLADGGCSGGAYDIIICDECHSXIDATTILGIGTVLDQAETAGARLVVLATATPPGSV TVPHPNIEEVALGX₂EGEIPFYGKAIPLX₃X₄IKGGRHLIFCHSKKKCDELAAKLRGMGLNAVAYY RGLDVSVIPTX₅GDVVVVATDALMTGYTGDFDSVIDCN (SEQ ID NO:58), where X₁ is Thr or Val; X₂ is Thr, Asn, Gln, His, or Ser; X₃ is Glu or Ala; X₄ is Val, Thr, Tyr, Phe, or Leu; and X₅ is Ser or Gln; and can have a length of 228 amino acids.

For example, a TP228 polypeptide can comprise an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LHAPTGSGKSTKVPAAYAAQGYKVLVLNPSVAATLGFGAYMSKAHGIDPNIRTGVRTITTGSPI TYSTYGKFLADGGCSGGAYDIIICDECHSTDATSILGIGTVLDQAETAGARLVVLATATPPGSVT VPHPNIEEVALSTTGEIPFYGKAIPLEVIKGGRHLIFCHSKKKCDELAAKLVALGINAVAYYRGL DVSVIPTSGDVVVVATDALMTGFTGDFDSVIDCN (SEQ ID NO:382); and can have a length of from 215 amino acids (aa) to 235 aa (e.g., 215 aa, 216 aa, 217 11, 218 aa, 219 aa, 220 aa, 221 aa, 222 aa, 223 aa, 224 aa, 225 aa, 226 aa, 227 aa, 228 aa, 229 aa, 230 aa, 231 aa, 232 aa, 233 aa, 234 aa, or 235 aa). In some cases, a TP228 polypeptide comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence: LHAPTGSGKSTKVPAAYAAQGYKVLVLNPSVAATLGFGAYMSKAHGIDPNIRTGVRTITTGSPI TYSTYGKFLADGGCSGGAYDIIICDECHSTDATSILGIGTVLDQAETAGARLVVLATATPPGSVT VPHPNIEEVALSTTGEIPFYGKAIPLEVIKGGRHLIFCHSKKKCDELAAKLVALGINAVAYYRGL DVSVIPTSGDVVVVATDALMTGFTGDFDSVIDCN (SEQ ID NO:382); and has a length of 228 amino acids.

### TP553 T-Cell Epitope Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP553 amino acid sequence depicted in FIG. 4; and has a length of about 553 amino acids.

### TP778 T-Cell Epitope Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP778 amino acid sequence depicted in FIG. 4; and has a length of about 778 amino acids.

### TP1987 T-Cell Epitope Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the TP1987 amino acid sequence depicted in FIG. 4; and has a length of about 1987 amino acids.

### TP465 Polypeptides

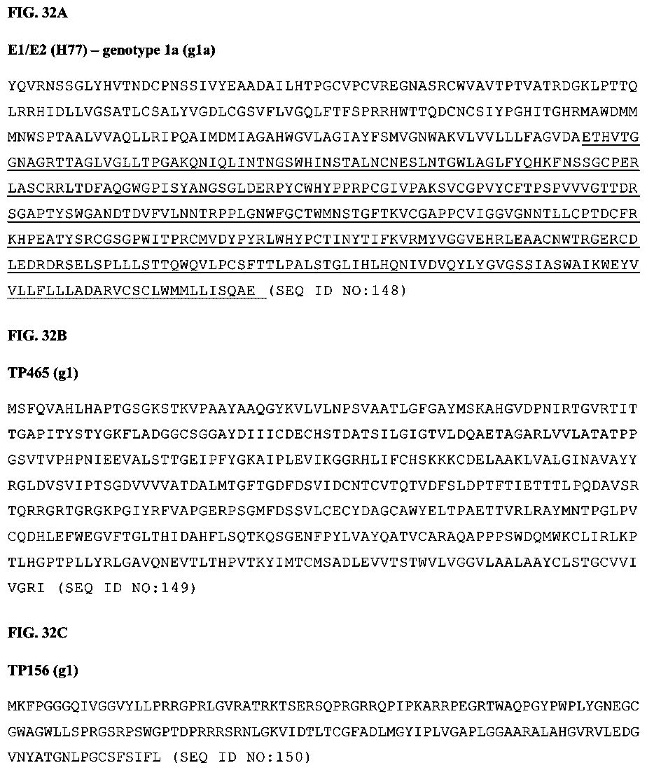

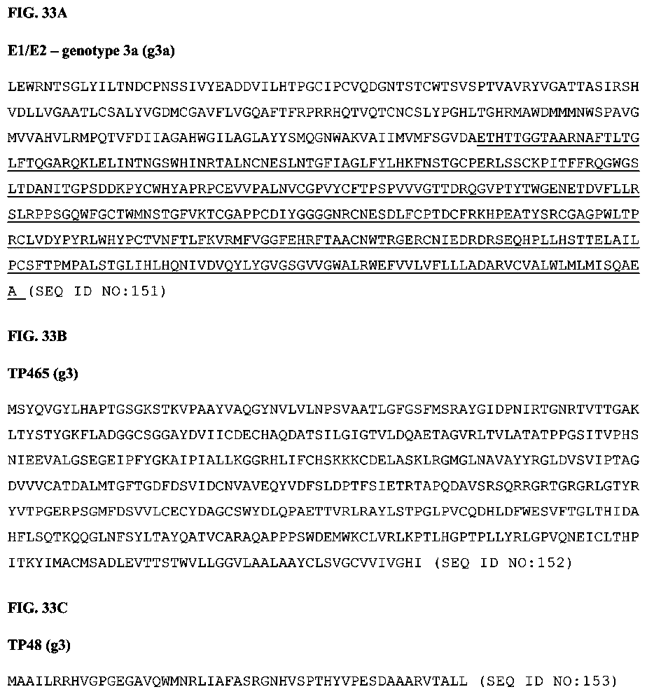

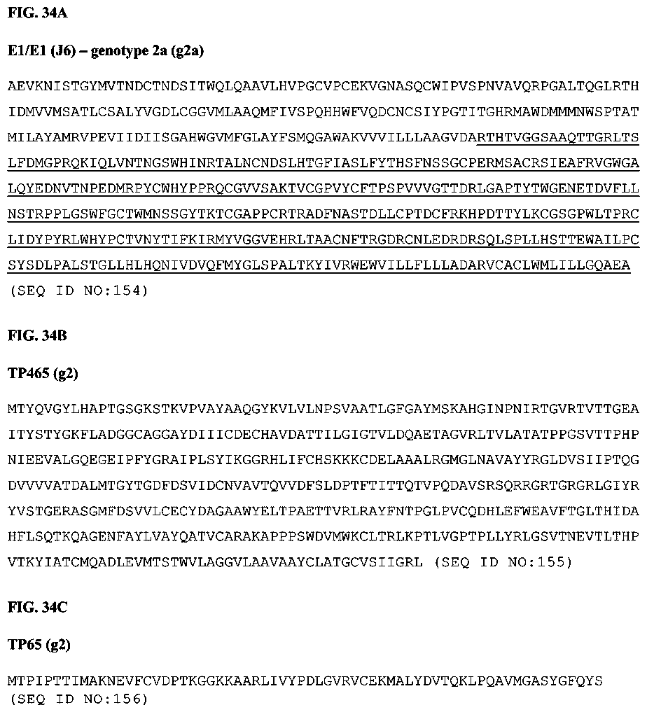

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to any one of the TP465 polypeptide amino acid sequences depicted in FIG. 32B, FIG. 33B, and FIG. 34B; and has a length of from 460 amino acids to 470 amino acids, e.g., has a length of 465 amino acids.

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence:

(SEQ ID NO: 383)
SFQVAHLHAPTGSGKSTKVPAAYAAQGYKVLVLNPSVAATLGFGAYMSK
AHGVDPNIRTGVRTITTGAPITYSTYGKFLADGGCSGGAYDIIICDECH
STDATSILGIGTVLDQAETAGARLVVLATATPPGSVTVPHPNIEEVALS
TTGEIPFYGKAIPLEVIKGGRHLIFCHSKKKCDELAAKLVALGINAVAY
YRGLDVSVIPTSGDVVVVATDALMTGFTGDFDSVIDCNTCVTQTVDFSL
DPTFTIETTTLPQDAVSRTQRRGRTGRGKPGIYRFVAPGERPSGMFDSS
VLCECYDAGCAWYELTPAETTVRLRAYMNTPGLPVCQDHLEFWEGVFTG
LTHIDAHFLSQTKQSGENFPYLVAYQATVCARAQAPPPSWDQMWKCLIR
LKPTLHGPTPLLYRLGAVQNEVTLTHPVTKYIMTCMSADLEVVTSTWVL
VGGVLAALAAYCLSTGCVVIVGRI;

and has a length of about 465 amino acids.

### TP156 Polypeptides

In some cases, a TP encoded by an RNA molecule of the present disclosure comprises an amino acid sequence having at least about 20%, at least about 25%, at least about 30%, at least about 35%, at least about 40%, at least about 45%, at least about 50%, at least about 60%, at least about 70%, at least about 75%, at least about 80%, at least about 85%, at least about 90%, at least about 95%, at least about 98%, at least about 99%, or 100%, amino acid sequence identity to the following amino acid sequence:

(SEQ ID NO: 384)
KFPGGGQIVGGVYLLPRRGPRLGVRATRKTSERSQPRGRRQPIPKARRP
EGRTWAQPGYPWPLYGNEGCGWAGWLLSPRGSRPSWGPTDPRRRSRNLG
KVIDTLTCGFADLMGYIPLVGAPLGGAARALAHGVRVLEDGVNYATGNL
PGCSFSIFL;

and has a length of about 165 amino acids.

### Multiple T-Cell Epitope Polypeptides in a Single Polypeptide Chain (Fusion Polypeptides)

In some cases, a heterologous polypeptide encoded in an RNA molecule of the present disclosure is a fusion polypeptide comprising 2, 3, 4, 5, 6, 7, 8, 9, or 10 of the aforementioned TPs in a single polypeptide chain. In other words, the heterologous polypeptide is in some cases a fusion polypeptide comprising 2, 3, 4, 5, 6, 7, 8, 9, or 10 of the TPs in a single polypeptide chain, where such TPs may be referred to as a “TP fusion polypeptide.”

For example, in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; b) a TP fusion polypeptide comprising, in order from N-terminus to C-terminus: i) a TP35-NS3 polypeptide, as described above (e.g., a polypeptide having the following amino acid sequence: KSTKVPAAYAAQGYKVLVLNPSVAATLGFGAYMSK (SEQ ID NO:102); and having a length of 35 amino acids); and ii) a TP50C polypeptide, as described above (e.g., a polypeptide having the following amino acid sequence: GVYLLPRRGPRLGVRATRKTSERSQPRGRRQPIPKARRSEGRSWAQPGYP (SEQ ID NO:101); and having a length of 50 amino acids. Thus, e.g., in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; b) a TP fusion polypeptide comprising a polypeptide having at least 20% (at least 20%, at least 30%, at least 40%, at least 50%, at least 60%, at least 70%, at least 80%, at least 90%, at least 95%, at least 98%, or 100%) amino acid identity to: KSTKVPAAYAAQGYKVLVLNPSVAATLGFGAYMSKGVYLLPRRGPRLGVRATRKTSERSQPR GRRQPIPKARRSEGRSWAQPGYP (SEQ ID NO:385) and having a length of 85 amino acids.

As another example, in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; b) a TP fusion polypeptide comprising, in order from N-terminus to C-terminus: i) a TP50C polypeptide, as described above (e.g., a polypeptide having the following amino acid sequence: GVYLLPRRGPRLGVRATRKTSERSQPRGRRQPIPKARRSEGRSWAQPGYP (SEQ ID NO:101); and having a length of 50 amino acids; and ii) a TP35-NS3 polypeptide, as described above (e.g., a polypeptide having the following amino acid sequence: KSTKVPAAYAAQGYKVLVLNPSVAATLGFGAYMSK (SEQ ID NO:102); and having a length of 35 amino acids). Thus, e.g., in some cases, a composition of the present disclosure comprises one or more RNA molecules comprising a nucleotide sequence encoding s: a) i) an HCV E1/E2 heterodimeric polypeptide; ii) an HCV E2 polypeptide; or iii) an HCV E1 polypeptide; b) a TP fusion polypeptide comprising a polypeptide having at least 20% (at least 20%, at least 30%, at least 40%, at least 50%, at least 60%, at least 70%, at least 80%, at least 90%, at least 95%, at least 98%, or 100%) amino acid identity to: GVYLLPRRGPRLGVRATRKTSERSQPRGRRQPIPKARRSEGRSWAQPGYPKSTKVPAAYAAQG YKVLVLNPSVAATLGFGAYMSK (SEQ ID NO:386) and having a length of 85 amino acids.

As another example, in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; b) a TP fusion polypeptide comprising, in order from N-terminus to C-terminus: i) a TP35-NS3 polypeptide, as described above (e.g., a polypeptide having the following amino acid sequence: KSTKVPAAYAAQGYKVLVLNPSVAATLGFGAYMSK (SEQ ID NO:102); and having a length of 35 amino acids); ii) a TP50C polypeptide, as described above (e.g., a polypeptide having the following amino acid sequence: GVYLLPRRGPRLGVRATRKTSERSQPRGRRQPIPKARRSEGRSWAQPGYP (SEQ ID NO:101); and having a length of 50 amino acids; and iii) a TP23 polypeptide, as described above (e.g., a polypeptide having the following amino acid sequence: DVVVVATDALMTGFTGDFDSVID (SEQ ID NO:256); and having a length of 23 amino acids). Thus, e.g., in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; b) a TP fusion polypeptide having at least 20% (at least 20%, at least 30%, at least 40%, at least 50%, at least 60%, at least 70%, at least 80%, at least 90%, at least 95%, at least 98%, or 100%) amino acid identity to: KSTKVPAAYAAQGYKVLVLNPSVAATLGFGAYMSKGVYLLPRRGPRLGVRATRKTSERSQPR GRRQPIPKARRSEGRSWAQPGYPDVVVVATDALMTGFTGDFDSVID (SEQ ID NO:387) and having a length of 108 amino acids.

As another example, in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; b) a TP fusion polypeptide comprising, in order from N-terminus to C-terminus: i) a TP35-NS3 polypeptide, as described above (e.g., a polypeptide having the following amino acid sequence: KSTKVPAAYAAQGYKVLVLNPSVAATLGFGAYMSK (SEQ ID NO:102); and having a length of 35 amino acids); ii) a TP50C polypeptide, as described above (e.g., a polypeptide having the following amino acid sequence: GVYLLPRRGPRLGVRATRKTSERSQPRGRRQPIPKARRSEGRSWAQPGYP (SEQ ID NO:101); and having a length of 50 amino acids; iii) a TP23 polypeptide, as described above (e.g., a polypeptide having the following amino acid sequence: DVVVVATDALMTGFTGDFDSVID (SEQ ID NO:256); and having a length of 23 amino acids); and iv) a TP27 polypeptide, as described above (e.g., a polypeptide having the following amino acid sequence: LEQIKGGRHLIFCHSKKKCDELAAKLT (SEQ ID NO:272); and having a length of 27 amino acids). Thus, e.g., in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; b) a TP fusion polypeptide comprising an amino acid sequence having at least 20% (at least 20%, at least 30%, at least 40%, at least 50%, at least 60%, at least 70%, at least 80%, at least 90%, at least 95%, at least 98%, or 100%) amino acid identity to: KSTKVPAAYAAQGYKVLVLNPSVAATLGFGAYMSKGVYLLPRRGPRLGVRATRKTSERSQPR GRRQPIPKARRSEGRSWAQPGYPDVVVVATDALMTGFTGDFDSVIDLEQIKGGRHLIFCHSKKK CDELAAKLT SEQ ID NO:388) and having a length of 135 amino acids.

As another example, in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; b) a TP fusion polypeptide comprising, in order from N-terminus to C-terminus: i) a TP35-NS3 polypeptide, as described above (e.g., a polypeptide having the following amino acid sequence: KSTKVPAAYAAQGYKVLVLNPSVAATLGFGAYMSK (SEQ ID NO:102); and having a length of 35 amino acids); ii) a TP50C polypeptide, as described above (e.g., a polypeptide having the following amino acid sequence: GVYLLPRRGPRLGVRATRKTSERSQPRGRRQPIPKARRSEGRSWAQPGYP (SEQ ID NO:101); and having a length of 50 amino acids; iii) a TP23 polypeptide, as described above (e.g., a polypeptide having the following amino acid sequence: DVVVVATDALMTGFTGDFDSVID (SEQ ID NO:256); and having a length of 23 amino acids); iv) a TP27 polypeptide, as described above (e.g., a polypeptide having the following amino acid sequence: LEQIKGGRHLIFCHSKKKCDELAAKLT (SEQ ID NO:272); and having a length of 27 amino acids); and v) a TP35-NS4 polypeptide, as described above (e.g., a polypeptide having the following amino acid sequence: ILRRHVGPGEGAVQWMNRLIAFASRGNHVSPTHYV (SEQ ID NO:287); and having a length of 35 amino acids. Thus, e.g., in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; b) a TP fusion polypeptide having at least 20% (at least 20%, at least 30%, at least 40%, at least 50%, at least 60%, at least 70%, at least 80%, at least 90%, at least 95%, at least 98%, or 100%) amino acid identity to: KSTKVPAAYAAQGYKVLVLNPSVAATLGFGAYMSKGVYLLPRRGPRLGVRATRKTSERSQPR GRRQPIPKARRSEGRSWAQPGYPDVVVVATDALMTGFTGDFDSVIDLEQIKGGRHLIFCHSKKK CDELAAKLTILRRHVGPGEGAVQWMNRLIAFASRGNHVSPTHYV (SEQ ID NO:389) and having a length of 170 amino acids.

As another example, in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; b) a TP fusion polypeptide comprising, in order from N-terminus to C-terminus: i) a TP35-NS3 polypeptide, as described above (e.g., a polypeptide having the following amino acid sequence: KSTKVPAAYAAQGYKVLVLNPSVAATLGFGAYMSK (SEQ ID NO:102); and having a length of 35 amino acids); ii) a TP50C polypeptide, as described above (e.g., a polypeptide having the following amino acid sequence: GVYLLPRRGPRLGVRATRKTSERSQPRGRRQPIPKARRSEGRSWAQPGYP (SEQ ID NO:101); and having a length of 50 amino acids; iii) a TP42 polypeptide as described above (e.g., a polypeptide having following amino acid sequence: GTEGEIPFYGKAIPLEQIKGGRHLIFCHSKKKCDELAAKLTG (SEQ ID NO:313); and having a length of 42 amino acids); and iv) a TP48 polypeptide as described above (e.g., a polypeptide having following amino acid sequence: ILRRHVGPGEGAVQWMNRLIAFASRGNHVSPTHYVPESDAAARVTQIL (SEQ ID NO:104); and having a length of 48 amino acids).

As another example, in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; and b) a TP fusion polypeptide comprising, in order from N-terminus to C-terminus: i) a TP35-NS3 polypeptide, as described above (e.g., a polypeptide having the following amino acid sequence: KSTKVPAAYAAQGYKVLVLNPSVAATLGFGAYMSK (SEQ ID NO:102); and having a length of 35 amino acids); ii) a TP50C polypeptide, as described above (e.g., a polypeptide having the following amino acid sequence: GVYLLPRRGPRLGVRATRKTSERSQPRGRRQPIPKARRSEGRSWAQPGYP (SEQ ID NO:101); and having a length of 50 amino acids; iii) a TP42 polypeptide as described above (e.g., a polypeptide having following amino acid sequence: GTEGEIPFYGKAIPLEQIKGGRHLIFCHSKKKCDELAAKLTG (SEQ ID NO:303); and having a length of 42 amino acids); iv) a TP48 polypeptide as described above (e.g., a polypeptide having following amino acid sequence: ILRRHVGPGEGAVQWMNRLIAFASRGNHVSPTHYVPESDAAARVTQIL (SEQ ID NO:104); and having a length of 48 amino acids); and v) a TP42-2 polypeptide, as described above (e.g., a polypeptide having the following amino acid sequence: KGGRHLIFCHSKKKCDELAAKLTGLGLNAVAYYRGLDVSVIP (SEQ ID NO:370); and having a length of 42 amino acids.

A TP fusion polypeptide encoded by an RNA molecule of the present disclosure can comprise: i) a TP50-C polypeptide having the amino acid sequence: GVYLLPRRGPRLGVRATRKTSERSQPRGRRQPIPKARRSEGRSWAQPGYP (SEQ ID NO:101); ii) a TP27 polypeptide having the amino acid sequence: LEQIKGGRHLIFCHSKKKCDELAAKLR (SEQ ID NO:103); iii) a TP48 polypeptide having the amino acid sequence: ILRRHVGPGEGAVQWMNRLIAFASRGNHVSPTHYVPESDAAARVTQIL (SEQ ID NO:104); and iv) a TP35-NS3 polypeptide having the amino acid sequence: KSTKVPAAYAAQGYKVLVLNPSVAATLGFGAYMSK (SEQ ID NO:102), where the TP50C-, TP27, TP48, and TP35-NS3 polypeptides are in any order in the TP fusion polypeptide.

As an example, in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; and b) a TP fusion polypeptide comprising, in order from N-terminus to C-terminus: i) a TP50-C polypeptide having the amino acid sequence: GVYLLPRRGPRLGVRATRKTSERSQPRGRRQPIPKARRSEGRSWAQPGYP (SEQ ID NO:101); ii) a TP35-NS3 polypeptide having the amino acid sequence: KSTKVPAAYAAQGYKVLVLNPSVAATLGFGAYMSK (SEQ ID NO:102); iii) a TP27 polypeptide having the amino acid sequence: LEQIKGGRHLIFCHSKKKCDELAAKLR (SEQ ID NO:103); and iv) a TP48 polypeptide having the amino acid sequence: ILRRHVGPGEGAVQWMNRLIAFASRGNHVSPTHYVPESDAAARVTQIL (SEQ ID NO:104).

As another example, in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; and b) a TP fusion polypeptide comprising, in order from N-terminus to C-terminus: i) a TP48 polypeptide having the amino acid sequence: ILRRHVGPGEGAVQWMNRLIAFASRGNHVSPTHYVPESDAAARVTQIL (SEQ ID NO:104); ii) a TP50-C polypeptide having the amino acid sequence: GVYLLPRRGPRLGVRATRKTSERSQPRGRRQPIPKARRSEGRSWAQPGYP (SEQ ID NO:101); iii) a TP35-NS3 polypeptide having the amino acid sequence: KSTKVPAAYAAQGYKVLVLNPSVAATLGFGAYMSK (SEQ ID NO:102); and iv) a TP27 polypeptide having the amino acid sequence: LEQIKGGRHLIFCHSKKKCDELAAKLR (SEQ ID NO:103).

As another example, in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; and b) a TP fusion polypeptide comprising, in order from N-terminus to C-terminus: i) a TP27 polypeptide having the amino acid sequence: LEQIKGGRHLIFCHSKKKCDELAAKLR (SEQ ID NO:103); ii) a TP48 polypeptide having the amino acid sequence: ILRRHVGPGEGAVQWMNRLIAFASRGNHVSPTHYVPESDAAARVTQIL (SEQ ID NO:104); iii) a TP50-C polypeptide having the amino acid sequence: GVYLLPRRGPRLGVRATRKTSERSQPRGRRQPIPKARRSEGRSWAQPGYP (SEQ ID NO:101); and iv) a TP35-NS3 polypeptide having the amino acid sequence: KSTKVPAAYAAQGYKVLVLNPSVAATLGFGAYMSK (SEQ ID NO:102).

As another example, in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; and b) a TP fusion polypeptide comprising, in order from N-terminus to C-terminus: i) a TP35-NS3 polypeptide having the amino acid sequence: KSTKVPAAYAAQGYKVLVLNPSVAATLGFGAYMSK (SEQ ID NO:102); ii) a TP27 polypeptide having the amino acid sequence: LEQIKGGRHLIFCHSKKKCDELAAKLR (SEQ ID NO:103); iii) a TP48 polypeptide having the amino acid sequence: ILRRHVGPGEGAVQWMNRLIAFASRGNHVSPTHYVPESDAAARVTQIL (SEQ ID NO:104); and iv) a TP50-C polypeptide having the amino acid sequence: GVYLLPRRGPRLGVRATRKTSERSQPRGRRQPIPKARRSEGRSWAQPGYP (SEQ ID NO:101).

As another example, in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; and b) a TP fusion polypeptide comprising, in order from N-terminus to C-terminus: i) a TP50-C polypeptide having the amino acid sequence: GVYLLPRRGPRLGVRATRKTSERSQPRGRRQPIPKARRSEGRSWAQPGYP (SEQ ID NO:101); ii) a TP27 polypeptide having the amino acid sequence: LEQIKGGRHLIFCHSKKKCDELAAKLR (SEQ ID NO:103); iii) a TP48 polypeptide having the amino acid sequence: ILRRHVGPGEGAVQWMNRLIAFASRGNHVSPTHYVPESDAAARVTQIL (SEQ ID NO:104); and iv) a TP35-NS3 polypeptide having the amino acid sequence: KSTKVPAAYAAQGYKVLVLNPSVAATLGFGAYMSK (SEQ ID NO:104).

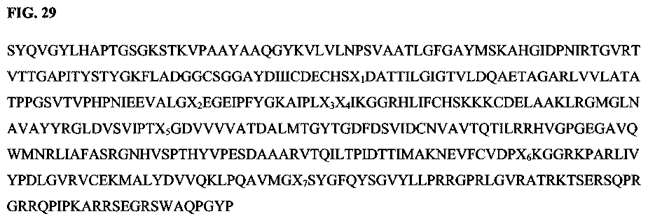

As another example, in some cases, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; and b) a TP fusion polypeptide comprising: i) a TP240 polypeptide; ii) a TP48 polypeptide; and iii) a TP65 polypeptide; iv) a TP50C polypeptide. The TP240 polypeptide, the TP48 polypeptide, the TP65 polypeptide, and the TP50C polypeptide can be in the TP fusion polypeptide in any order. For example, a composition of the present disclosure comprises one or more RNA molecules comprising a nucleotide sequence encoding: a) i) an HCV E1/E2 heterodimeric polypeptide; ii) an HCV E2 polypeptide; or iii) an HCV E1 polypeptide; and b) a TP fusion polypeptide comprising, in order from N-terminus to C-terminus: i) a TP240 polypeptide; ii) a TP48 polypeptide; and iii) a TP65 polypeptide; iv) a TP50C polypeptide. As an example, the TP fusion polypeptide can comprise an amino acid sequence having at least about 90%, at least about 95%, at least about 98%, at least about 99%, or 100%, amino acid sequence identity, to the amino acid sequence depicted in FIG. 29, where X₁ is T, V, or Q, X₂ is T, N, Q, H, or S, X₃ is E, S, or A, X₄ is V, T, Y, F, or L, X₅ is S, Q, or T, X₆ is E, T, or V, and X₇ is S, A, or P; where the TP fusion polypeptide has a length of from 410 amino acids to 415 amino acids (e.g., 410, 411, 412, 413, 414, or 415 amino acids). In some cases, the TP fusion polypeptide has a length of 410 amino acids.

### Combinations

As noted above, an RNA molecule of the present disclosure can comprise (in addition to a nucleotide sequence(s) encoding an HCV E1 and/or E2 polypeptide) nucleotide sequences encoding 2, 3, 4, 5, 6, 7, 8, 9, or 10 different T-cell epitope polypeptides as the heterologous polypeptide.

Combination #1. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% amino acid sequence identity to: KSTKVPAAYAAQGYKVLVLNPSVAATLGFGAYMSK (SEQ ID NO:102); and c) a second TP comprising an amino acid sequence having at least 20% amino acid sequence identity to: GVYLLPRRGPRLGVRATRKTSERSQPRGRRQPIPKARRSEGRSWAQPGYP (SEQ ID NO:101). The first TP can have a length of 35 amino acids; and the second TP can have a length of 50 amino acids. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; and c) a second TP comprising the following amino acid sequence: GVYLLPRRGPRLGVRATRKTSERSQPRGRRQPIPKARRSEGRSWAQPGYP (SEQ ID NO:101) and having a length of 50 amino acids.

Combination #2. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSK (SEQ ID NO:102); c) a second T-cell epitope polypeptide comprising an amino acid sequence having at least 20% amino acid sequence identity to: DVVVVATDALMTGFTGDFDSVID (SEQ ID NO:256); and d) a pharmaceutically acceptable excipient. The first TP can have a length of 35 amino acids; and the second T-cell epitope polypeptide can have a length of 23 amino acids. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; and c) a second TP comprising the following amino acid sequence: DVVVVATDALMTGFTGDFDSVID (SEQ ID NO:256) and having a length of 23 amino acids.

Combination #3. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSK (SEQ ID NO:102); and c) a second TP comprising an amino acid sequence having at least 20% amino acid sequence identity to: LEQIKGGRHLIFCHSKKKCDELAAKLT (SEQ ID NO:272). The first TP can have a length of 35 amino acids; and the second TP can have a length of 27 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; c) a second TP comprising the following amino acid sequence: LEQIKGGRHLIFCHSKKKCDELAAKLT (SEQ ID NO:272) and having a length of 27 amino acids.

Combination #4. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSK (SEQ ID NO:102); and c) a second TP comprising an amino acid sequence having at least 20% amino acid sequence identity to: ILRRHVGPGEGAVQWMNRLIAFASRGNHVSPTHYV (SEQ ID NO:287). The first TP can have a length of 35 amino acids; and the second TP can have a length of 35 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; and c) a second TP comprising the following amino acid sequence: ILRRHVGPGEGAVQWMNRLIAFASRGNHVSPTHYV (SEQ ID NO:287) and having a length of 35 amino acids.

Combination #5. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: GVYLLPRRGPRLGVRATRKTSERSQPRGRRQPIPKARRSEGRSWAQPGYP (SEQ ID NO:101); and c) a second TP comprising an amino acid sequence having at least 20% amino acid sequence identity to: DVVVVATDALMTGFTGDFDSVID (SEQ ID NO:256). The first TP can have a length of 50 amino acids; and the second TP can have a length of 23 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: GVYLLPRRGPRLGVRATRKTSERSQPRGRRQPIPKARRSEGRSWAQPGYP (SEQ ID NO:101) and having a length of 50 amino acids; and c) a second T-cell epitope polypeptide comprising the following amino acid sequence: DVVVVATDALMTGFTGDFDSVID (SEQ ID NO:256) and having a length of 23 amino acids;

Combination #6. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: GVYLLPRRGPRLGVRATRKTSERSQPRGRRQPIPKARRSEGRSWAQPGYP (SEQ ID NO:101); and c) a second T-cell epitope polypeptide comprising an amino acid sequence having at least 20% amino acid sequence identity to: LEQIKGGRHLIFCHSKKKCDELAAKLT (SEQ ID NO:272). The first T-cell epitope polypeptide can have a length of 50 amino acids; and the second T-cell epitope polypeptide can have a length of 27 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first T-cell epitope polypeptide comprising the following amino acid sequence: GVYLLPRRGPRLGVRATRKTSERSQPRGRRQPIPKARRSEGRSWAQPGYP (SEQ ID NO:101) and having a length of 50 amino acids; and c) a second T-cell epitope polypeptide comprising the following amino acid sequence: LEQIKGGRHLIFCHSKKKCDELAAKLT (SEQ ID NO:272) and having a length of 27 amino acids.

Combination #7. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: GVYLLPRRGPRLGVRATRKTSERSQPRGRRQPIPKARRSEGRSWAQPGYP (SEQ ID NO:101); and c) a second TP comprising an amino acid sequence having at least 20% amino acid sequence identity to: ILRRHVGPGEGAVQWMNRLIAFASRGNHVSPTHYV (SEQ ID NO:287). The first T-cell epitope polypeptide can have a length of 50 amino acids; and the second TP can have a length of 35 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: GVYLLPRRGPRLGVRATRKTSERSQPRGRRQPIPKARRSEGRSWAQPGYP (SEQ ID NO:101) and having a length of 50 amino acids; and c) a second TP comprising the following amino acid sequence: ILRRHVGPGEGAVQWMNRLIAFASRGNHVSPTHYV (SEQ ID NO:287) and having a length of 35 amino acids.

Combination #8. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: DVVVVATDALMTGFTGDFDSVID (SEQ ID NO:256); and c) a second TP comprising an amino acid sequence having at least 20% amino acid sequence identity to: LEQIKGGRHLIFCHSKKKCDELAAKLT (SEQ ID NO:272). The first TP can have a length of 23 amino acids; and the second T-cell epitope polypeptide can have a length of 27 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: DVVVVATDALMTGFTGDFDSVID (SEQ ID NO:256) and having a length of 23 amino acids; and c) a second TP comprising the following amino acid sequence: LEQIKGGRHLIFCHSKKKCDELAAKLT (SEQ ID NO:272) and having a length of 27 amino acids.

Combination #9. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: DVVVVATDALMTGFTGDFDSVID (SEQ ID NO:256); and c) a second T-cell epitope polypeptide comprising an amino acid sequence having at least 20% amino acid sequence identity to: ILRRHVGPGEGAVQWMNRLIAFASRGNHVSPTHYV (SEQ ID NO:287). The first TP can have a length of 23 amino acids; and the second TP can have a length of 35 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: DVVVVATDALMTGFTGDFDSVID (SEQ ID NO:256) and having a length of 23 amino acids; and c) a second TP comprising the following amino acid sequence: ILRRHVGPGEGAVQWMNRLIAFASRGNHVSPTHYV (SEQ ID NO:287) and having a length of 35 amino acids.

Combination #10. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: LEQIKGGRHLIFCHSKKKCDELAAKLT (SEQ ID NO:272); and c) a second TP comprising an amino acid sequence having at least 20% amino acid sequence identity to: ILRRHVGPGEGAVQWMNRLIAFASRGNHVSPTHYV (SEQ ID NO:287). The first T-cell epitope polypeptide can have a length of 27 amino acids; and the second T-cell epitope polypeptide can have a length of 35 amino acids. As an example, an immunogenic composition of the present disclosure can comprise: a) i) an HCV E1/E2 heterodimeric polypeptide; ii) an HCV E2 polypeptide; or iii) an HCV E1 polypeptide; b) a first TP comprising the following amino acid sequence: LEQIKGGRHLIFCHSKKKCDELAAKLT (SEQ ID NO:272) and having a length of 27 amino acids; and c) a second TP comprising the following amino acid sequence: ILRRHVGPGEGAVQWMNRLIAFASRGNHVSPTHYV (SEQ ID NO:287) and having a length of 35 amino acids.

Combination #11. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSK (SEQ ID NO:102); c) a second TP comprising an amino acid sequence having at least 20% amino acid sequence identity to: GVYLLPRRGPRLGVRATRKTSERSQPRGRRQPIPKARRSEGRSWAQPGYP (SEQ ID NO:101); and d) a third TP comprising an amino acid sequence having at least 20% amino acid sequence identity to: DVVVVATDALMTGFTGDFDSVID (SEQ ID NO:256). The first TP can have a length of 35 amino acids; the second TP can have a length of 50 amino acids; and the third TP can have a length of 23 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; c) a second TP comprising the following amino acid sequence: GVYLLPRRGPRLGVRATRKTSERSQPRGRRQPIPKARRSEGRSWAQPGYP (SEQ ID NO:101) and having a length of 50 amino acids; and d) a third TP comprising an amino acid sequence having at least 20% amino acid sequence identity to: DVVVVATDALMTGFTGDFDSVID (SEQ ID NO:256) and having a length of 23 amino acids.

Combination #12. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSK (SEQ ID NO:102); c) a second TP comprising an amino acid sequence having at least 20% amino acid sequence identity to: GVYLLPRRGPRLGVRATRKTSERSQPRGRRQPIPKARRSEGRSWAQPGYP (SEQ ID NO:101); and d) a third TP comprising an amino acid sequence having at least 20% amino acid sequence identity to: LEQIKGGRHLIFCHSKKKCDELAAKLT (SEQ ID NO:272). The first TP can have a length of 35 amino acids; the second TP can have a length of 50 amino acids; and the third TP can have a length of 27 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; c) a second TP comprising the following amino acid sequence: GVYLLPRRGPRLGVRATRKTSERSQPRGRRQPIPKARRSEGRSWAQPGYP (SEQ ID NO:101) and having a length of 50 amino acids; and d) a third TP comprising an amino acid sequence having at least 20% amino acid sequence identity to: LEQIKGGRHLIFCHSKKKCDELAAKLT (SEQ ID NO:272) and having a length of 27 amino acids.

Combination #13. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSK (SEQ ID NO:102); c) a second TP comprising an amino acid sequence having at least 20% amino acid sequence identity to: GVYLLPRRGPRLGVRATRKTSERSQPRGRRQPIPKARRSEGRSWAQPGYP (SEQ ID NO:101); and d) a third TP comprising an amino acid sequence having at least 20% amino acid sequence identity to: ILRRHVGPGEGAVQWMNRLIAFASRGNHVSPTHYV (SEQ ID NO:287). The first TP can have a length of 35 amino acids; the second TP can have a length of 50 amino acids; and the third TP can have a length of 35 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; c) a second TP comprising the following amino acid sequence: GVYLLPRRGPRLGVRATRKTSERSQPRGRRQPIPKARRSEGRSWAQPGYP (SEQ ID NO:101) and having a length of 50 amino acids; and d) a third TP comprising an amino acid sequence having at least 20% amino acid sequence identity to: ILRRHVGPGEGAVQWMNRLIAFASRGNHVSPTHYV (SEQ ID NO:287) and having a length of 35 amino acids.

Combination #14. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: GVYLLPRRGPRLGVRATRKTSERSQPRGRRQPIPKARRSEGRSWAQPGYP (SEQ ID NO:101); c) a second TP comprising an amino acid sequence having at least 20% amino acid sequence identity to: DVVVVATDALMTGFTGDFDSVID (SEQ ID NO:256); and d) a third TP comprising an amino acid sequence having at least 20% amino acid sequence identity to: LEQIKGGRHLIFCHSKKKCDELAAKLT (SEQ ID NO:272). The first TP can have a length of 50 amino acids; the second TP can have a length of 23 amino acids; and the third TP can have a length of 27 amino acids. As an example an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: GVYLLPRRGPRLGVRATRKTSERSQPRGRRQPIPKARRSEGRSWAQPGYP (SEQ ID NO:101) and having a length of 50 amino acids; c) a second TP comprising the following amino acid sequence: DVVVVATDALMTGFTGDFDSVID (SEQ ID NO:256) and having a length of 23 amino acids; and d) a third TP comprising an amino acid sequence having at least 20% amino acid sequence identity to: LEQIKGGRHLIFCHSKKKCDELAAKLT (SEQ ID NO:272) and having a length of 27 amino acids.

Combination #15. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: GVYLLPRRGPRLGVRATRKTSERSQPRGRRQPIPKARRSEGRSWAQPGYP (SEQ ID NO:101); c) a second TP comprising an amino acid sequence having at least 20% amino acid sequence identity to: DVVVVATDALMTGFTGDFDSVID (SEQ ID NO:256); and d) a third TP comprising an amino acid sequence having at least 20% amino acid sequence identity to: ILRRHVGPGEGAVQWMNRLIAFASRGNHVSPTHYV (SEQ ID NO:287). The first TP can have a length of 50 amino acids; the second TP can have a length of 23 amino acids; and the third TP can have a length of 35 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: GVYLLPRRGPRLGVRATRKTSERSQPRGRRQPIPKARRSEGRSWAQPGYP (SEQ ID NO:101) and having a length of 50 amino acids; c) a second TP comprising the following amino acid sequence: DVVVVATDALMTGFTGDFDSVID (SEQ ID NO:256) and having a length of 23 amino acids; and d) a third TP comprising an amino acid sequence having at least 20% amino acid sequence identity to: ILRRHVGPGEGAVQWMNRLIAFASRGNHVSPTHYV (SEQ ID NO:287) and having a length of 35 amino acids.

Combination #16. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: DVVVVATDALMTGFTGDFDSVID (SEQ ID NO:256); c) a second TP comprising an amino acid sequence having at least 20% amino acid sequence identity to: LEQIKGGRHLIFCHSKKKCDELAAKLT (SEQ ID NO:272); and d) a third TP comprising an amino acid sequence having at least 20% amino acid sequence identity to: ILRRHVGPGEGAVQWMNRLIAFASRGNHVSPTHYV (SEQ ID NO:287). The first TP can have a length of 23 amino acids; the second TP can have a length of 27 amino acids; and the third TP can have a length of 35 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: DVVVVATDALMTGFTGDFDSVID (SEQ ID NO:256) and having a length of 23 amino acids; c) a second TP comprising the following amino acid sequence: LEQIKGGRHLIFCHSKKKCDELAAKLT (SEQ ID NO:272) and having a length of 27 amino acids; and d) a third TP comprising an amino acid sequence having at least 20% amino acid sequence identity to: ILRRHVGPGEGAVQWMNRLIAFASRGNHVSPTHYV (SEQ ID NO:287) and having a length of 35 amino acids.

Combination #17. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSK (SEQ ID NO:102); c) a second TP comprising an amino acid sequence having at least 20% amino acid sequence identity to: GVYLLPRRGPRLGVRATRKTSERSQPRGRRQPIPKARRSEGRSWAQPGYP (SEQ ID NO:101); d) a third TP comprising an amino acid sequence having at least 20% amino acid sequence identity to: DVVVVATDALMTGFTGDFDSVID (SEQ ID NO:256); and e) a fourth TP comprising an amino acid sequence having at least 20% amino acid sequence identity to: LEQIKGGRHLIFCHSKKKCDELAAKLT (SEQ ID NO:272). The first TP can have a length of 35 amino acids; the second TP can have a length of 50 amino acids; the third TP can have a length of 23 amino acids; and the fourth TP can have a length of 27 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; c) a second TP comprising the following amino acid sequence: GVYLLPRRGPRLGVRATRKTSERSQPRGRRQPIPKARRSEGRSWAQPGYP (SEQ ID NO:101) and having a length of 50 amino acids; d) a third TP comprising an amino acid sequence having at least 20% amino acid sequence identity to: DVVVVATDALMTGFTGDFDSVID (SEQ ID NO:256) and having a length of 23 amino acids; and e) a fourth TP comprising an amino acid sequence having at least 20% amino acid sequence identity to: LEQIKGGRHLIFCHSKKKCDELAAKLT (SEQ ID NO:272) and having a length of 27 amino acids.

Combination #18. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSK (SEQ ID NO:102); c) a second TP comprising an amino acid sequence having at least 20% amino acid sequence identity to: GVYLLPRRGPRLGVRATRKTSERSQPRGRRQPIPKARRSEGRSWAQPGYP (SEQ ID NO:101); d) a third TP comprising an amino acid sequence having at least 20% amino acid sequence identity to: DVVVVATDALMTGFTGDFDSVID (SEQ ID NO:256); and e) a fourth TP comprising an amino acid sequence having at least 20% amino acid sequence identity to: ILRRHVGPGEGAVQWMNRLIAFASRGNHVSPTHYV (SEQ ID NO:287). The first TP can have a length of 35 amino acids; the second TP can have a length of 50 amino acids; the third TP can have a length of 23 amino acids; and the fourth TP can have a length of 35 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; c) a second TP comprising the following amino acid sequence: GVYLLPRRGPRLGVRATRKTSERSQPRGRRQPIPKARRSEGRSWAQPGYP (SEQ ID NO:101) and having a length of 50 amino acids; d) a third TP comprising an amino acid sequence having at least 20% amino acid sequence identity to: DVVVVATDALMTGFTGDFDSVID (SEQ ID NO:256) and having a length of 23 amino acids; and e) a fourth TP comprising an amino acid sequence having at least 20% amino acid sequence identity to: ILRRHVGPGEGAVQWMNRLIAFASRGNHVSPTHYV (SEQ ID NO:287) and having a length of 35 amino acids.

Combination #19. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSK (SEQ ID NO:102); c) a second TP comprising an amino acid sequence having at least 20% amino acid sequence identity to: GVYLLPRRGPRLGVRATRKTSERSQPRGRRQPIPKARRSEGRSWAQPGYP (SEQ ID NO:101); d) a third TP comprising an amino acid sequence having at least 20% amino acid sequence identity to: LEQIKGGRHLIFCHSKKKCDELAAKLT (SEQ ID NO:272); and e) a fourth TP comprising an amino acid sequence having at least 20% amino acid sequence identity to: ILRRHVGPGEGAVQWMNRLIAFASRGNHVSPTHYV (SEQ ID NO:287). The first TP can have a length of 35 amino acids; the second TP can have a length of 50 amino acids; the third TP can have a length of 27 amino acids; and the fourth TP can have a length of 35 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; c) a second TP comprising the following amino acid sequence: GVYLLPRRGPRLGVRATRKTSERSQPRGRRQPIPKARRSEGRSWAQPGYP (SEQ ID NO:101) and having a length of 50 amino acids; d) a third TP comprising an amino acid sequence having at least 20% amino acid sequence identity to: LEQIKGGRHLIFCHSKKKCDELAAKLT (SEQ ID NO:272) and having a length of 27 amino acids; and e) a fourth TP comprising an amino acid sequence having at least 20% amino acid sequence identity to: ILRRHVGPGEGAVQWMNRLIAFASRGNHVSPTHYV (SEQ ID NO:287) and having a length of 35 amino acids.

Combination #20. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSK (SEQ ID NO:102); c) a second TP comprising an amino acid sequence having at least 20% amino acid sequence identity to: GVYLLPRRGPRLGVRATRKTSERSQPRGRRQPIPKARRSEGRSWAQPGYP (SEQ ID NO:101); d) a third TP comprising an amino acid sequence having at least 20% amino acid sequence identity to: DVVVVATDALMTGFTGDFDSVID (SEQ ID NO:256); e) a fourth TP comprising an amino acid sequence having at least 20% amino acid sequence identity to: LEQIKGGRHLIFCHSKKKCDELAAKLT (SEQ ID NO:272); and f) a fifth TP comprising an amino acid sequence having at least 20% amino acid sequence identity to: ILRRHVGPGEGAVQWMNRLIAFASRGNHVSPTHYV (SEQ ID NO:287). The first TP can have a length of 35 amino acids; the second TP can have a length of 50 amino acids; the third TP can have a length of 23 amino acids; the fourth TP can have a length of 27 amino acids; and the fifth TP can have a length of 35 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; c) a second TP comprising the following amino acid sequence: GVYLLPRRGPRLGVRATRKTSERSQPRGRRQPIPKARRSEGRSWAQPGYP (SEQ ID NO:101) and having a length of 50 amino acids; d) a third TP comprising an amino acid sequence having at least 20% amino acid sequence identity to: DVVVVATDALMTGFTGDFDSVID (SEQ ID NO:256) and having a length of 23 amino acids; e) a fourth TP comprising an amino acid sequence having at least 20% amino acid sequence identity to: LEQIKGGRHLIFCHSKKKCDELAAKLT (SEQ ID NO:272) and having a length of 27 amino acids; and f) a fifth TP comprising an amino acid sequence having at least 20% amino acid sequence identity to: ILRRHVGPGEGAVQWMNRLIAFASRGNHVSPTHYV (SEQ ID NO:287) and having a length of 35 amino acids.

Combination #21. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSK (SEQ ID NO:102); and c) a second TP comprising an amino acid sequence having at least 20% amino acid sequence identity to: GTEGEIPFYGKAIPLEQIKGGRHLIFCHSKKKCDELAAKLTG(K)n (SEQ ID NO:390), where n is an integer from 2 to 10 (e.g., where n is 2, 3, 4, 5, 6, 7, 8, 9, or 10). The first TP can have a length of 35 amino acids; and the second TP can have a length of from 44 amino acids to 52 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; and c) a second TP comprising the following amino acid sequence: GTEGEIPFYGKAIPLEQIKGGRHLIFCHSKKKCDELAAKLTGKKK (SEQ ID NO:391) and has a length of 45 amino acids.

Combination #22. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSK (SEQ ID NO:102); and c) a second TP comprising an amino acid sequence having at least 20% amino acid sequence identity to: LNAVAYYRGLDVSVIPTSGDVVVVATDALMTGFTGDFDSVIDCNV(K)n (SEQ ID NO:392), where n is an integer from 2 to 10 (e.g., where n is 2, 3, 4, 5, 6, 7, 8, 9, or 10). The first TP can have a length of 35 amino acids; and the second TP can have a length of from 47 amino acids to 57 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; and c) a second TP comprising the following amino acid sequence: LNAVAYYRGLDVSVIPTSGDVVVVATDALMTGFTGDFDSVIDCNVKKK (SEQ ID NO:334) and having a length of 48 amino acids.

Composition #23. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSK (SEQ ID NO:102); and c) a second TP comprising an amino acid sequence having at least 20% amino acid sequence identity to: ILRRHVGPGEGAVQWMNRLIAFASRGNHVSPTHYVPESDASARVTQIL(Lys)n (SEQ ID NO:393), where n is an integer from 2 to 10 (e.g., where n is 2, 3, 4, 5, 6, 7, 8, 9, or 10). The first TP can have a length of 35 amino acids; and the second TP can have a length of from 50 amino acids to 58 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; c) a second TP comprising the following amino acid sequence: ILRRHVGPGEGAVQWMNRLIAFASRGNHVSPTHYVPESDASARVTQILKKK (SEQ ID NO:394) and having a length of 51 amino acids.

Combination #24. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: GTEGEIPFYGKAIPLEQIKGGRHLIFCHSKKKCDELAAKLTG(K)n (SEQ ID NO:390), where n is an integer from 2 to 10 (e.g., where n is 2, 3, 4, 5, 6, 7, 8, 9, or 10); and c) a second TP comprising an amino acid sequence having at least 20% amino acid sequence identity to: LNAVAYYRGLDVSVIPTSGDVVVVATDALMTGFTGDFDSVIDCNV(K)n (SEQ ID NO:392), where n is an integer from 2 to 10 (e.g., where n is 2, 3, 4, 5, 6, 7, 8, 9, or 10). The first TP can have a length of from 44 amino acids to 52 amino acids; and the second TP can have a length of from 47 amino acids to 57 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: GTEGEIPFYGKAIPLEQIKGGRHLIFCHSKKKCDELAAKLTGKKK (SEQ ID NO:391) and having a length of 45 amino acids; and c) a second TP comprising the following amino acid sequence: LNAVAYYRGLDVSVIPTSGDVVVVATDALMTGFTGDFDSVIDCNVKKK (SEQ ID NO:394) and having a length of 48 amino acids.

Combination #25. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: GTEGEIPFYGKAIPLEQIKGGRHLIFCHSKKKCDELAAKLTG(K)n (SEQ ID NO:390), where n is an integer from 2 to 10 (e.g., where n is 2, 3, 4, 5, 6, 7, 8, 9, or 10); and c) a second TP comprising an amino acid sequence having at least 20% amino acid sequence identity to: ILRRHVGPGEGAVQWMNRLIAFASRGNHVSPTHYVPESDASARVTQIL (SEQ ID NO:338). The first TP can have a length of from 44 amino acids to 52 amino acids; and the second TP can have a length of 48 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: GTEGEIPFYGKAIPLEQIKGGRHLIFCHSKKKCDELAAKLTGKKK (SEQ ID NO:391) and having a length of 45 amino acids; and c) a second TP comprising the following amino acid sequence: ILRRHVGPGEGAVQWMNRLIAFASRGNHVSPTHYVPESDASARVTQIL(Lys)n (SEQ ID NO:393), where n is an integer from 2 to 10 (e.g., where n is 2, 3, 4, 5, 6, 7, 8, 9, or 10).

Combination #26. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: GTEGEIPFYGKAIPLEQIKGGRHLIFCHSKKKCDELAAKLTG(K)n (SEQ ID NO:390), where n is an integer from 2 to 10 (e.g., where n is 2, 3, 4, 5, 6, 7, 8, 9, or 10); and c) a second TP comprising an amino acid sequence having at least 20% amino acid sequence identity to: GVYLLPRRGPRLGVRATRKTSERSQPRGRRQPIPKARRSEGRSWAQPGYP (SEQ ID NO:101). The first TP can have a length of from 44 amino acids to 52 amino acids; and the second TP can have a length of 50 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: GTEGEIPFYGKAIPLEQIKGGRHLIFCHSKKKCDELAAKLTGKKK (SEQ ID NO:391) and having a length of 45 amino acids; and c) a second TP comprising the following amino acid sequence: GVYLLPRRGPRLGVRATRKTSERSQPRGRRQPIPKARRSEGRSWAQPGYP (SEQ ID NO:101) and having a length of 50 amino acids.

Combination #27. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: ILRRHVGPGEGAVQWMNRLIAFASRGNHVSPTHYVPESDASARVTQIL(Lys)n (SEQ ID NO:393), where n is an integer from 2 to 10 (e.g., where n is 2, 3, 4, 5, 6, 7, 8, 9, or 10); and c) a second TP comprising an amino acid sequence having at least 20% amino acid sequence identity to: GVYLLPRRGPRLGVRATRKTSERSQPRGRRQPIPKARRSEGRSWAQPGYP (SEQ ID NO:101).

The first TP can have a length of from 50 amino acids to 58 amino acids; and the second TP can have a length of 50 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: ILRRHVGPGEGAVQWMNRLIAFASRGNHVSPTHYVPESDASARVTQILKKK (SEQ ID NO:394) and having a length of 51 amino acids; and c) a second TP comprising the following amino acid sequence: GVYLLPRRGPRLGVRATRKTSERSQPRGRRQPIPKARRSEGRSWAQPGYP (SEQ ID NO:101) and having a length of 50 amino acids.

Combination #28. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSKKKK (SEQ ID NO:395); and c) a second TP comprising an amino acid sequence having at least 20% amino acid sequence identity to: GTEGEIPFYGKAIPLEQIKGGRHLIFCHSKKKCDELAAKLTG(K)n (SEQ ID NO:390), where n is an integer from 2 to 10 (e.g., where n is 2, 3, 4, 5, 6, 7, 8, 9, or 10). The first TP can have a length of 38 amino acids; and the second TP can have a length of from 44 amino acids to 52 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSKKKK (SEQ ID NO:395) and having a length of 38 amino acids; and c) a second TP comprising the following amino acid sequence: GTEGEIPFYGKAIPLEQIKGGRHLIFCHSKKKCDELAAKLTGKKK (SEQ ID NO:391) and having a length of 45 amino acids.

Combination #29. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSKKKK (SEQ ID NO:395); and c) a second TP comprising an amino acid sequence having at least 20% amino acid sequence identity to: LNAVAYYRGLDVSVIPTSGDVVVVATDALMTGFTGDFDSVIDCNVKKK (SEQ ID NO:334). The first TP can have a length of 38 amino acids; and the second TP can have a length of 48 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSKKKK (SEQ ID NO:395) and having a length of 38 amino acids; and c) a second TP comprising the following amino acid sequence: LNAVAYYRGLDVSVIPTSGDVVVVATDALMTGFTGDFDSVIDCNVKKK (SEQ ID NO:334) and having a length of 48 amino acids.

Combination #30. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSKKKK (SEQ ID NO:395); and c) a second TP comprising an amino acid sequence having at least 20% amino acid sequence identity to: ILRRHVGPGEGAVQWMNRLIAFASRGNHVSPTHYVPESDASARVTQILKKK (SEQ ID NO:394). The first TP can have a length of 38 amino acids; and the second T-cell epitope polypeptide can have a length of 51 amino acids. As an example, an RNA molecule of the present disclosure comprises nucleotide sequences encoding a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSKKKK (SEQ ID NO:395) and having a length of 38 amino acids; and c) a second TP comprising the following amino acid sequence: ILRRHVGPGEGAVQWMNRLIAFASRGNHVSPTHYVPESDASARVTQILKKK (SEQ ID NO:394) and having a length of 51 amino acids.

Combination #31. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSKKKK (SEQ ID NO:395); and c) a second TP comprising an amino acid sequence having at least 20% amino acid sequence identity to: GVYLLPRRGPRLGVRATRKTSERSQPRGRRQPIPKARRSEGRSWAQPGYP (SEQ ID NO:101). The first TP can have a length of 38 amino acids; and the second TP can have a length of 50 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSKKKK (SEQ ID NO:395) and having a length of 38 amino acids; and c) a second TP comprising the following amino acid sequence: GVYLLPRRGPRLGVRATRKTSERSQPRGRRQPIPKARRSEGRSWAQPGYP (SEQ ID NO:101) and having a length of 50 amino acids.

Combination #32. In some cases, an RNA molecule of the present disclosure comprises nucleotide sequences encoding 5 different TPs. Thus, in some cases, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: KSTKVPAAYAAQGYKVLVLNPSVAATLGFGAYMSK (SEQ ID NO:102), and having length of from 35 amino acids to 45 amino acids; c) a second TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GTEGEIPFYGKAIPLEQIKGGRHLIFCHSKKKCDELAAKLTG(K)n (SEQ ID NO:390), where n is an integer from 2 to 10 (e.g., where n is 2, 3, 4, 5, 6, 7, 8, 9, or 10), and having a length of from 44 amino acids to 52 amino acids; d) a third TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: LNAVAYYRGLDVSVIPTSGDVVVVATDALMTGFTGDFDSVIDCNVKKK (SEQ ID NO:334), and having a length of from 48 amino acids to 55 amino acids; e) a fourth TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: ILRRHVGPGEGAVQWMNRLIAFASRGNHVSPTHYVPESDASARVTQIL(Lys)n (SEQ ID NO:393), where n is an integer from 2 to 10 (e.g., where n is 2, 3, 4, 5, 6, 7, 8, 9, or 10), and having a length of from 50 amino acids to 58 amino acids; and f) a fifth TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GVYLLPRRGPRLGVRATRKTSERSQPRGRRQPIPKARRSEGRSWAQPGYP (SEQ ID NO:101), and having a length of from 50 amino acids to 60 amino acids.

Combination #33. In some cases, an RNA molecule of the present disclosure comprises nucleotide sequences encoding 5 different TPs. Thus, in some cases, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: KSTKVPAAYAAQGYKVLVLNPSVAATLGFGAYMSKKKK (SEQ ID NO:395), and having a length of from 38 amino acids to 45 amino acids; c) a second TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GTEGEIPFYGKAIPLEQIKGGRHLIFCHSKKKCDELAAKLTG(K)n (SEQ ID NO:390), where n is an integer from 2 to 10 (e.g., where n is 2, 3, 4, 5, 6, 7, 8, 9, or 10), and having a length of from 44 amino acids to 52 amino acids; d) a third TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: LNAVAYYRGLDVSVIPTSGDVVVVATDALMTGFTGDFDSVIDCNVKKK (SEQ ID NO:334), and having a length of from 48 amino acids to 55 amino acids; e) a fourth TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: ILRRHVGPGEGAVQWMNRLIAFASRGNHVSPTHYVPESDASARVTQILKKK (SEQ ID NO:394), and having a length of from 51 amino acids to 58 amino acids; and f) a fifth TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GVYLLPRRGPRLGVRATRKTSERSQPRGRRQPIPKARRSEGRSWAQPGYP (SEQ ID NO:101), and having a length of from 50 amino acids to 60 amino acids.

Combination #34. In some cases, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: KSTKVPAAYAAQGYKVLVLNPSVAATLGFGAYMSK (SEQ ID NO:102), and having length of from 35 amino acids to 45 amino acids; c) a second TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GTEGEIPFYGKAIPLEQIKGGRHLIFCHSKKKCDELAAKLTG(K)n (SEQ ID NO:390), where n is an integer from 2 to 10 (e.g., where n is 2, 3, 4, 5, 6, 7, 8, 9, or 10), and having a length of from 44 amino acids to 52 amino acids; d) a third TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: LNAVAYYRGLDVSVIPTSGDVVVVATDALMTGFTGDFDSVID (SEQ ID NO:337), and having a length of from 42 amino acids to 52 amino acids; e) a fourth TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: ILRRHVGPGEGAVQWMNRLIAFASRGNHVSPTHYVPESDASARVTQIL(Lys)n (SEQ ID NO:393), where n is an integer from 2 to 10 (e.g., where n is 2, 3, 4, 5, 6, 7, 8, 9, or 10), and having a length of from 50 amino acids to 58 amino acids; and f) a fifth TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GVYLLPRRGPRLGVRATRKTSERSQPRGRRQPIPKARRSEGRSWAQPGYP (SEQ ID NO:101), and having a length of from 50 amino acids to 60 amino acids. For example, in some cases, the fourth TP comprises the following amino acid sequence: ILRRHVGPGEGAVQWMNRLIAFASRGNHVSPTHYVPESDASARVTQILKKK (SEQ ID NO:394) and has a length of 51 amino acids.

Combination #35. In some cases, an RNA molecule of the present disclosure comprises nucleotide sequences encoding 4 different TPs. Thus, in some cases, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: KSTKVPAAYAAQGYKVLVLNPSVAATLGFGAYMSK (SEQ ID NO:102), and having length of from 35 amino acids to 45 amino acids; c) a second TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GTEGEIPFYGKAIPLEQIKGGRHLIFCHSKKKCDELAAKLTG(K)n (SEQ ID NO:390), where n is an integer from 2 to 10 (e.g., where n is 2, 3, 4, 5, 6, 7, 8, 9, or 10), and having a length of from 44 amino acids to 52 amino acids; d) a third TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: ILRRHVGPGEGAVQWMNRLIAFASRGNHVSPTHYVPESDASARVTQIL(Lys)n (SEQ ID NO:393), where n is an integer from 2 to 10 (e.g., where n is 2, 3, 4, 5, 6, 7, 8, 9, or 10), and having a length of from 50 amino acids to 58 amino acids; and e) a fourth TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GVYLLPRRGPRLGVRATRKTSERSQPRGRRQPIPKARRSEGRSWAQPGYP (SEQ ID NO:101), and having a length of from 50 amino acids to 60 amino acids. For example, in some cases, the third TP comprises the following amino acid sequence: ILRRHVGPGEGAVQWMNRLIAFASRGNHVSPTHYVPESDASARVTQILKKK (SEQ ID NO:394) and has a length of 51 amino acids.

Combination #36. In some cases, an RNA molecule of the present disclosure comprises nucleotide sequences encoding 4 different TPs. Thus, in some cases, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first T-cell epitope polypeptide comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: KSTKVPAAYAAQGYKVLVLNPSVAATLGFGAYMSK(K)n (SEQ ID NO:396), where n is an integer from 2 to 10 (e.g., where n is 2, 3, 4, 5, 6, 7, 8, 9, or 10) and having a length of from 37 amino acids to 45 amino acids; c) a second TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GTEGEIPFYGKAIPLEQIKGGRHLIFCHSKKKCDELAAKLTG(K)n (SEQ ID NO:390), where n is an integer from 2 to 10 (e.g., where n is 2, 3, 4, 5, 6, 7, 8, 9, or 10), and having a length of from 44 amino acids to 52 amino acids; d) a third TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: ILRRHVGPGEGAVQWMNRLIAFASRGNHVSPTHYVPESDASARVTQILKKK (SEQ ID NO:394), and having a length of from 51 amino acids to 58 amino acids; and e) a fourth TP comprising an amino acid sequence having at least 20% (at least 20%, at least 30%, at least 40%, at least 50%, at least 60%, at least 70%, at least 80%, at least 90%, at least 95%, at least 98%, at least 99%, or 100%) amino acid sequence identity to: GVYLLPRRGPRLGVRATRKTSERSQPRGRRQPIPKARRSEGRSWAQPGYP (SEQ ID NO:101), and having a length of from 50 amino acids to 60 amino acids.

Combination #37. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 80% amino acid sequence identity to: KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, where the TP35-NS3 T-cell epitope polypeptide has a length of from 28 amino acids to 35 amino acids; c) a second TP comprising an amino acid sequence having at least 80% amino acid sequence identity to: GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₂ is S, T, H, or A; X₁₂ is A or G; and X₁₃ is Q or K, where the TP50C T-cell epitope polypeptide has a length of from 40 amino acids to 50 amino acids; and d) a third TP comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), where X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, where n is an integer from 2 to 10, and wherein the TP48 T-cell epitope polypeptide has a length of from 38 amino acids to 48 amino acids. For example, in some cases an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the amino acid sequence: KSTKVPAAYAAQGYKVLVLNPSVAATLGFGAYMSK (SEQ ID NO:102), and having a length of 35 amino acids; c) a second TP comprising the amino acid sequence: GVYLLPRRGPRLGVRATRKTSERSQPRGRRQPIPKARRSEGRSWAQPGYP (SEQ ID NO:101), and having a length of 50 amino acids; and d) a third TP comprising the amino acid sequence: ILRRHVGPGEGAVQWMNRLIAFASRGNHVSPTHYVPESDASARVTQILKKK (SEQ ID NO:394) and having a length of 51 amino acids.

Combination #38. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSK (SEQ ID NO:102); c) a second TP comprising an amino acid sequence having at least 20% amino acid sequence identity to: GVYLLPRRGPRLGVRATRKTSERSQPRGRRQPIPKARRSEGRSWAQPGYP (SEQ ID NO:101); d) a third TP comprising an amino acid sequence having at least 20% amino acid sequence identity to: GTEGEIPFYGKAIPLEQIKGGRHLIFCHSKKKCDELAAKLTG (SEQ ID NO:313); e) a fourth TP comprising an amino acid sequence having at least 20% amino acid sequence identity to: KGGRHLIFCHSKKKCDELAAKLTGLGLNAVAYYRGLDVSVIP (SEQ ID NO:370); and f) a fifth TP comprising an amino acid sequence having at least 20% amino acid sequence identity to: ILRRHVGPGEGAVQWMNRLIAFASRGNHVSPTHYVPESDASARVTQIL (SEQ ID NO:338). The first TP can have a length of 35 amino acids; the second TP can have a length of 50 amino acids; the third TP can have a length of 42 amino acids; the fourth TP can have a length of 42 amino acids; and the fifth TP can have a length of 48 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; c) a second TP comprising the following amino acid sequence: GVYLLPRRGPRLGVRATRKTSERSQPRGRRQPIPKARRSEGRSWAQPGYP (SEQ ID NO:101) and having a length of 50 amino acids; d) a third TP comprising an amino acid sequence having at least 20% amino acid sequence identity to: GTEGEIPFYGKAIPLEQIKGGRHLIFCHSKKKCDELAAKLTG (SEQ ID NO:313) and having a length of 42 amino acids; e) a fourth TP comprising an amino acid sequence having at least 20% amino acid sequence identity to: KGGRHLIFCHSKKKCDELAAKLTGLGLNAVAYYRGLDVSVIP (SEQ ID NO:370) and having a length of 42 amino acids; f) a fifth TP comprising an amino acid sequence having at least 20% amino acid sequence identity to: ILRRHVGPGEGAVQWMNRLIAFASRGNHVSPTHYVPESDASARVTQIL (SEQ ID NO:338) and having a length of 48 amino acids.

Combination #39. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising an amino acid sequence having at least 20% amino acid sequence identity to: KSTKVPAAYAAQGYKVLVLNPSVAATLGFGAYMSK (SEQ ID NO:102); c) a second TP comprising an amino acid sequence having at least 20% amino acid sequence identity to: GVYLLPRRGPRLGVRATRKTSERSQPRGRRQPIPKARRSEGRSWAQPGYP (SEQ ID NO:101); and d) a third TP comprising an amino acid sequence having at least 20% amino acid sequence identity to: GTEGEIPFYGKAIPLEQIKGGRHLIFCHSKKKCDELAAKLTG (SEQ ID NO:313);. The first TP can have a length of 35 amino acids; the second TP can have a length of 50 amino acids; and the third TP can have a length of 42 amino acids. As an example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP comprising the following amino acid sequence: KSTKVPAAYAAQGYKVLVLNPSVAATLGFGAYMSK (SEQ ID NO:102) and having a length of 35 amino acids; c) a second TP comprising the following amino acid sequence: GVYLLPRRGPRLGVRATRKTSERSQPRGRRQPIPKARRSEGRSWAQPGYP (SEQ ID NO:101) and having a length of 50 amino acids; d) a third TP comprising an amino acid sequence having at least 20% amino acid sequence identity to: GTEGEIPFYGKAIPLEQIKGGRHLIFCHSKKKCDELAAKLTG (SEQ ID NO:313) and having a length of 42 amino acids.

Combination #40. As one example, an RNA molecule of the present disclosure comprises nucleotide sequences encoding: a) i) an HCV E1/E2 heterodimeric polypeptide; ii) an HCV E2 polypeptide; or iii) an HCV E1 polypeptide; b) a first TP, where the first TP is a TP50-C TP having the amino acid sequence set out in Table 1; c) a second TP, where the second TP is a TP35-NS3 TP having the amino acid sequence set out in Table 1; d) a third TP, where the third TP is a TP27 TP having the amino acid sequence set out in Table 1; and e) a fourth TP, where the fourth TP is a TP48 TP having the amino acid sequence set out in Table 1.

TABLE 1

| TP | Amino acid sequence |
|---|---|
| TP50-C | GVYLLPRRGPRLGVRATRKTSERSQPRGRRQPIPKARRSEGRSWAQPGYP |
| | (SEQ ID NO: 101) |
| TP35- | KSTKVPAAYAAQGYKVLVLNPSVAATLGFGAYMSK (SEQ ID NO: 102) |
| NS3 | |
| TP27 | LEQIKGGRHLIFCHSKKKCDELAAKLR (SEQ ID NO: 103) |
| TP48 | ILRRHVGPGEGAVQWMNRLIAFASRGNHVSPTHYVPESDAAARVTQIL (SEQ |
| | ID NO: 104) |

### Compositions Comprising Two or More Different RNA Molecules

A composition of the present disclosure can include a single type of RNA; i.e., all of the RNA molecules in the composition can encode the same polypeptides (e.g., HCV E1 and/or E2 polypeptides; optionally also one or more heterologous polypeptides). Alternatively, a composition of the present disclosure can include multiple different RNA molecules, each encoding a different polypeptide or different combination of polypeptides.

For example, a composition of the present disclosure can comprise: a) a first RNA molecule comprising a nucleotide sequence encoding an HCV E1 and/or E2 polypeptide of a first genotype; b) a second RNA molecule comprising a nucleotide sequence encoding an HCV E1 and/or E2 polypeptide of a second genotype; and c) a third RNA molecule comprising a nucleotide sequence encoding an HCV E1 and/or E2 polypeptide of a third genotype. As another example, as illustrated schematically in FIG. 5A-5B, a composition of the present disclosure can comprise: a) a first RNA molecule comprising a nucleotide sequence encoding an HCV E1 and/or E2 polypeptide of a first genotype; b) a second RNA molecule comprising a nucleotide sequence encoding an HCV E1 and/or E2 polypeptide of a second genotype; c) a third RNA molecule comprising a nucleotide sequence encoding an HCV E1 and/or E2 polypeptide of a third genotype; and d) a fourth RNA molecule comprising a nucleotide sequence encoding one or more heterologous polypeptides (e.g., one or more TPs) or a heterologous fusion polypeptide (e.g., a fusion polypeptide comprising two or more (e.g., 1, 2, 3, 4, 5, 6, 7, 8, 9, or 10 TPs).

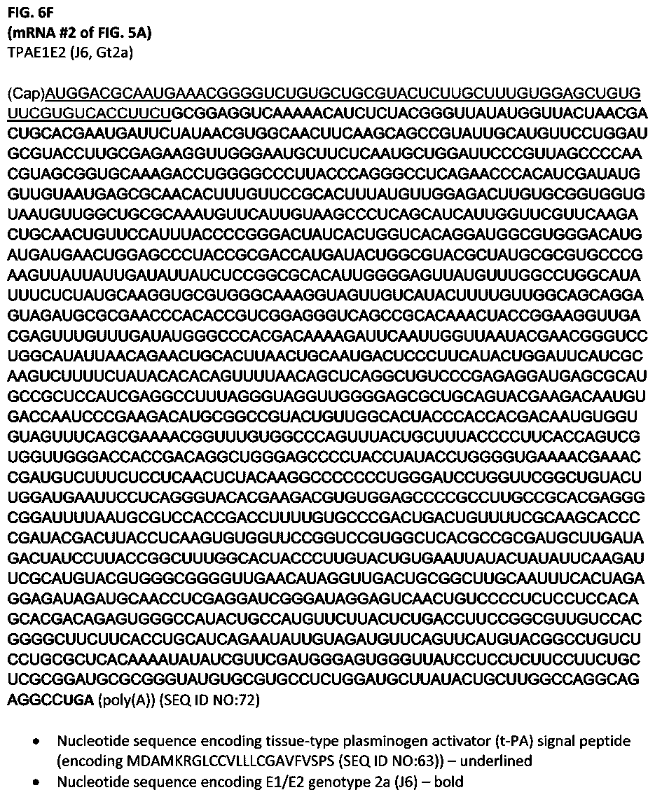

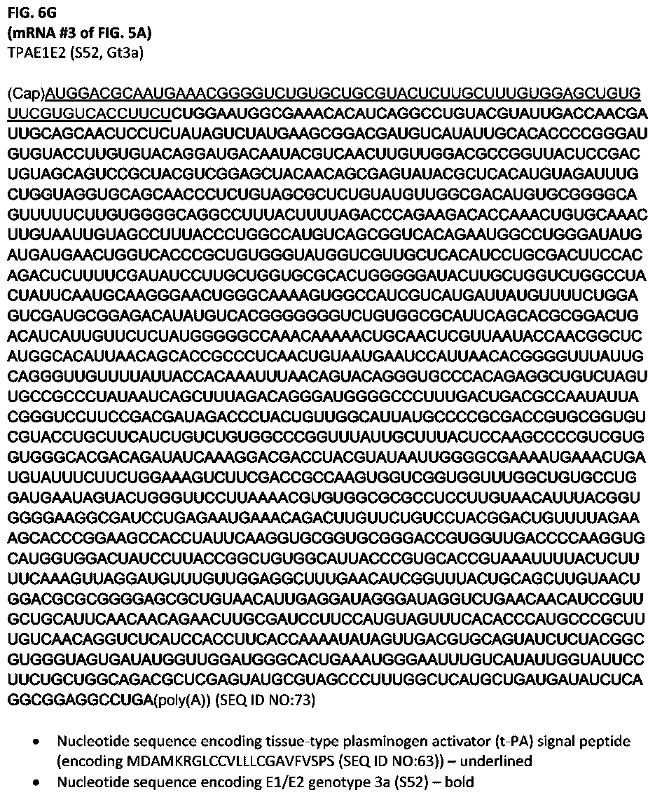

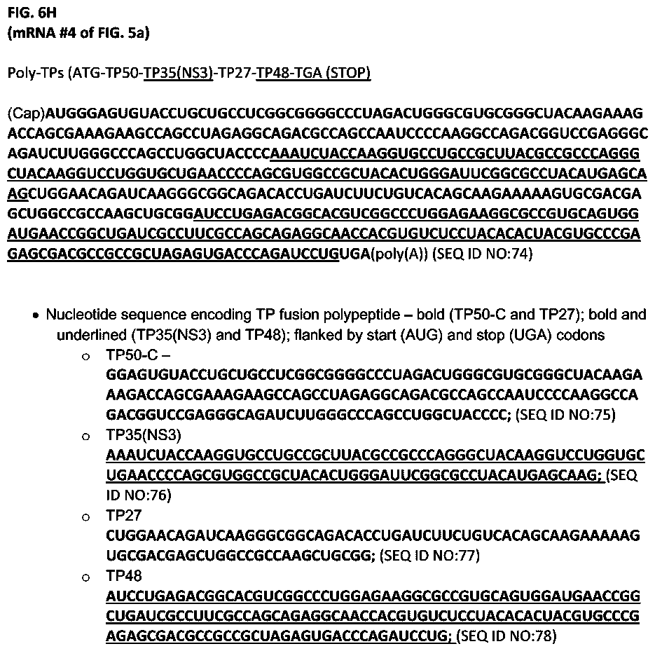

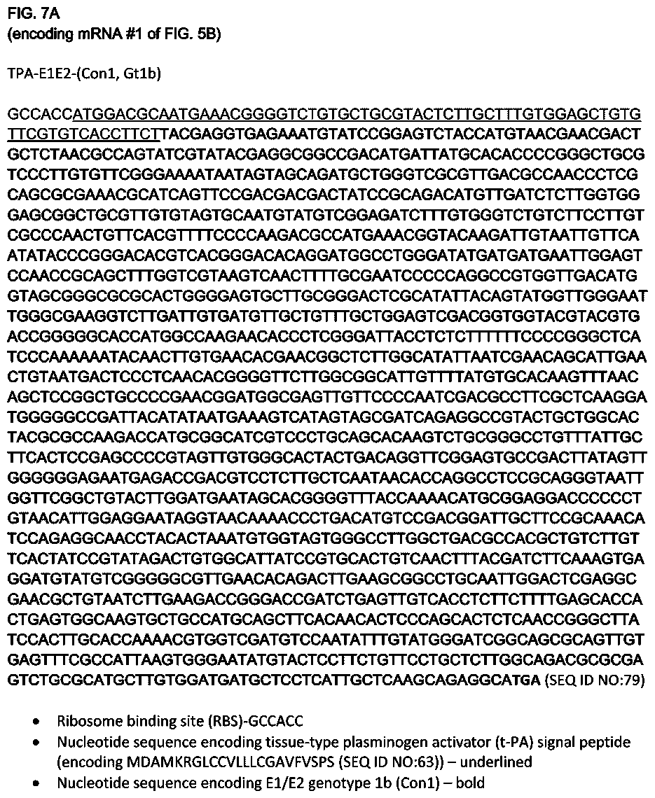

As an example, as depicted in FIG. 6A-6D and FIG. 6E-6H, a composition of the present disclosure can comprise: a) a first RNA molecule comprising a nucleotide sequence encoding HCV E1 and HCV E2 polypeptides of genotype 1a and having a heterologous signal peptide (e.g.,, a tissue-type plasminogen activator (t-PA) signal peptide); b) a second RNA molecule comprising a nucleotide sequence encoding HCV E1 and HCV E2 polypeptides of genotype 2a and having a heterologous signal peptide (e.g., t-PA); c) a third RNA molecule comprising a nucleotide sequence encoding HCV E1 and HCV E2 polypeptides of genotype 3a and having a heterologous signal peptide (e.g., t-PA); and d) a fourth RNA molecule comprising a nucleotide sequence encoding a fusion polypeptide comprising two or more TPs (e.g., TP50-C; TP35-NS3; TP27; and TP48). FIG. 6A-6D depict DNA molecules encoding mRNA molecules depicted in FIG. 6E-6H, respectively. The nucleotide sequences in FIG. 6A-6D, therefore, can be DNA molecules encoding mRNA molecules, or, where the Ts are replaced with Us, can be mRNA molecules (as set out in FIG. 6E-6H). The DNA molecules can include a polyadenylation signal. The mRNA molecules set out in FIG. 6E-6H can include a Cap sequence and can include a poly(A) tail.

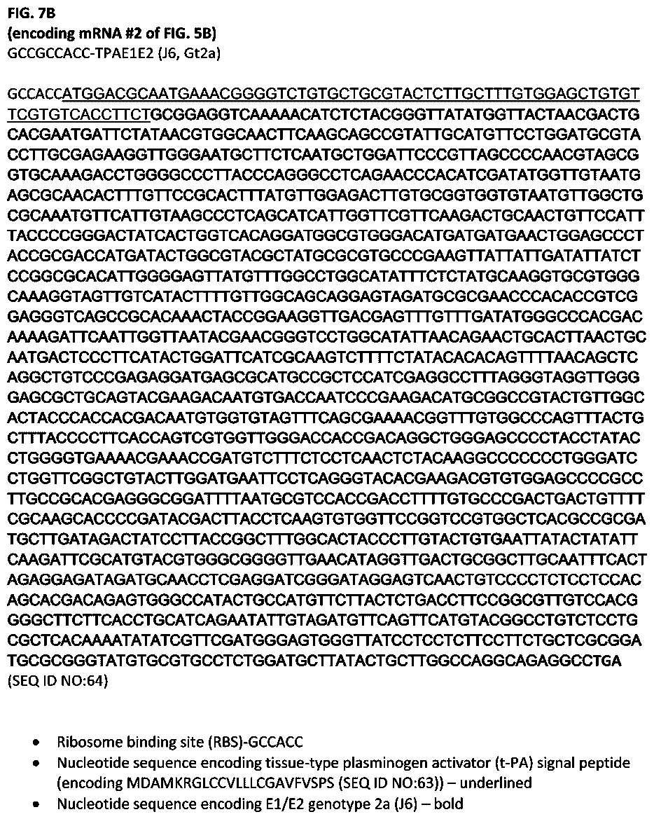

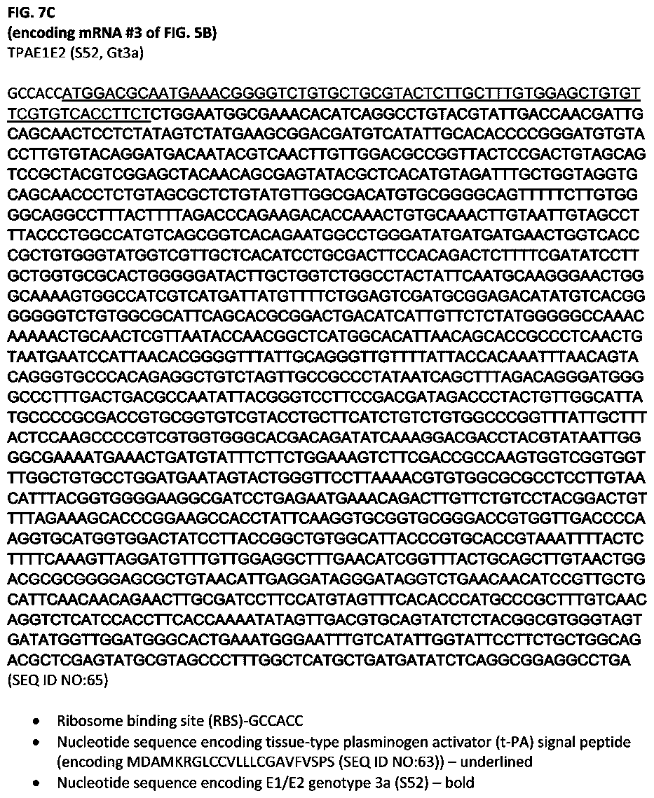

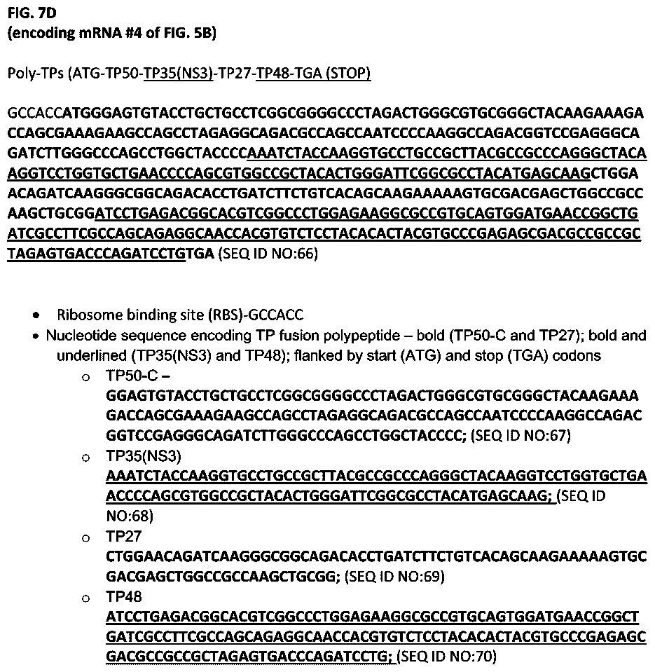

As another example, as depicted in FIG. 7A-7D, a composition of the present disclosure can comprise: a) a first RNA molecule comprising a nucleotide sequence encoding HCV E1 and HCV E2 polypeptides of genotype 1b and having a heterologous signal peptide (e.g., t-PA); b) a second RNA molecule comprising a nucleotide sequence encoding HCV E1 and HCV E2 polypeptides of genotype 2a and having a heterologous signal peptide (e.g., t-PA); c) a third RNA molecule comprising a nucleotide sequence encoding HCV E1 and HCV E2 polypeptides of genotype 3 and having a heterologous signal peptide (e.g., t-PA); and d) a fourth RNA molecule comprising a nucleotide sequence encoding a fusion polypeptide comprising two or more TPs (e.g., TP50-C; TP35-NS3; TP27; and TP48). The nucleotide sequences in FIG. 7A-7D can be DNA molecules encoding mRNA molecules, or, where the Ts are replaced with Us, can be mRNA molecules. The DNA molecules can include a polyadenylation signal. The mRNA molecules can include a Cap sequence and can include a poly(A) tail.

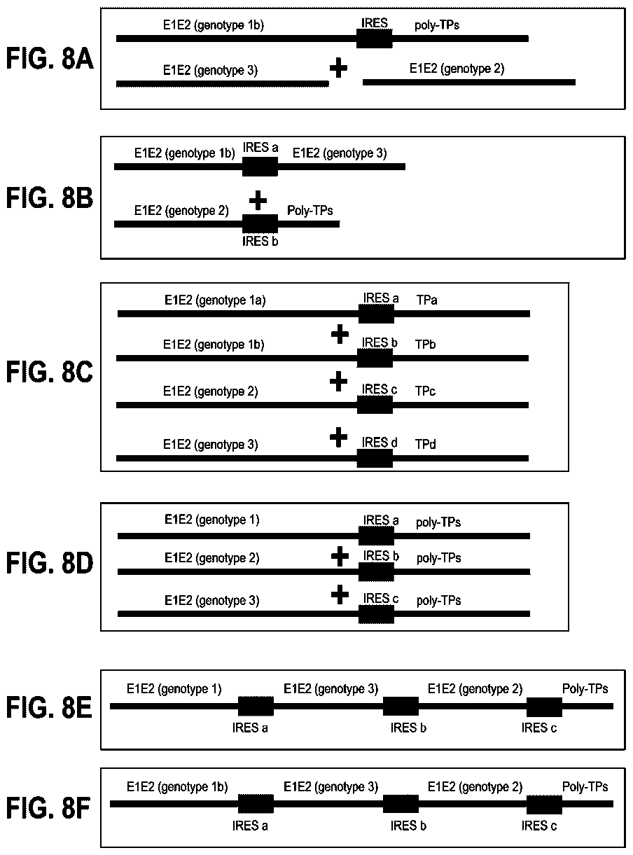

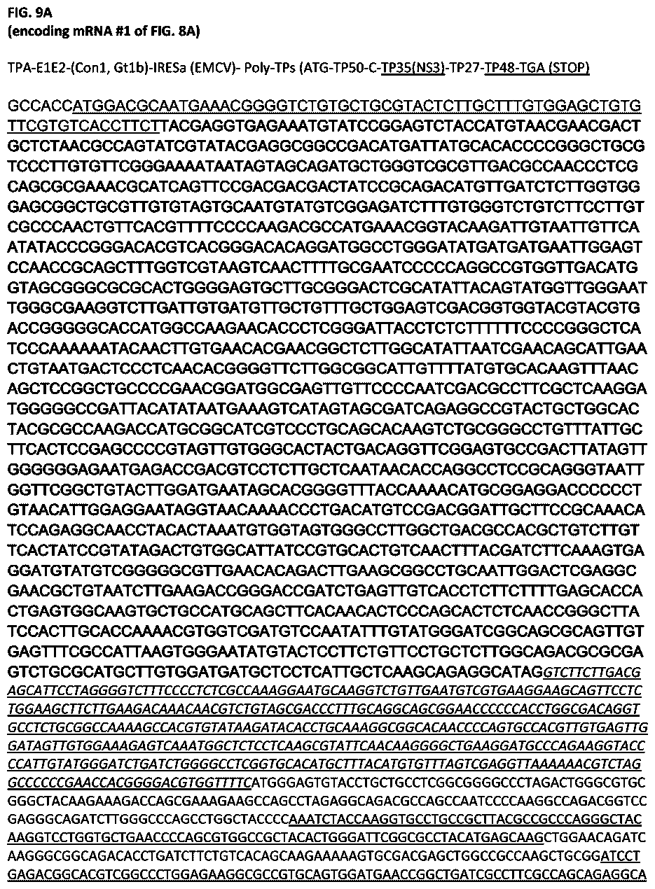

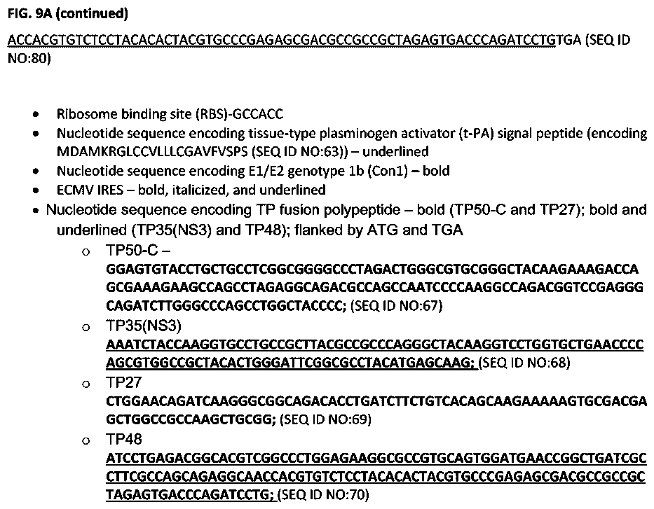

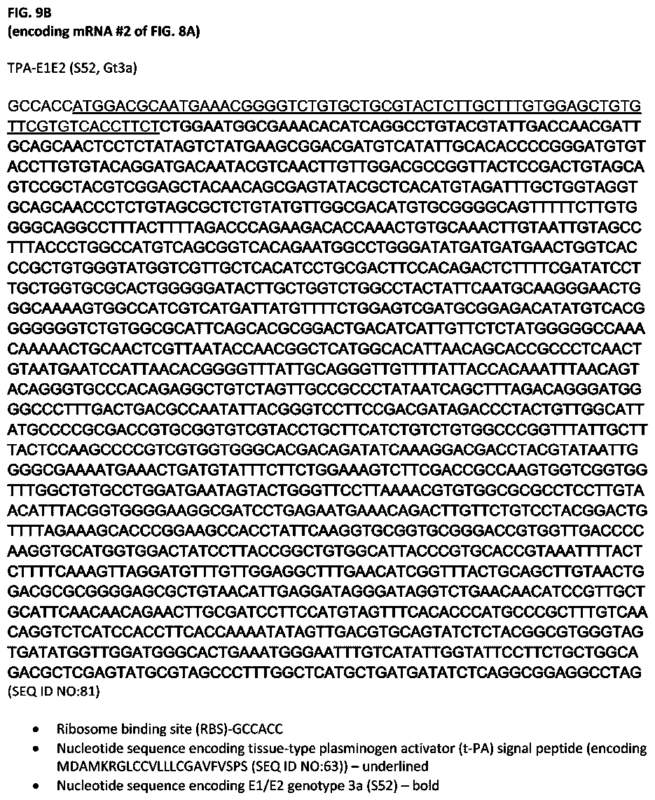

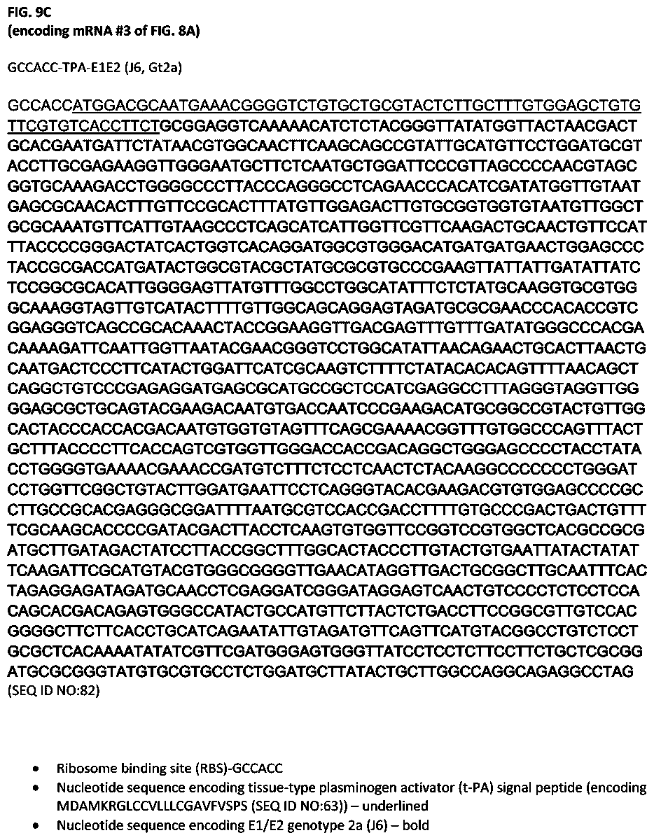

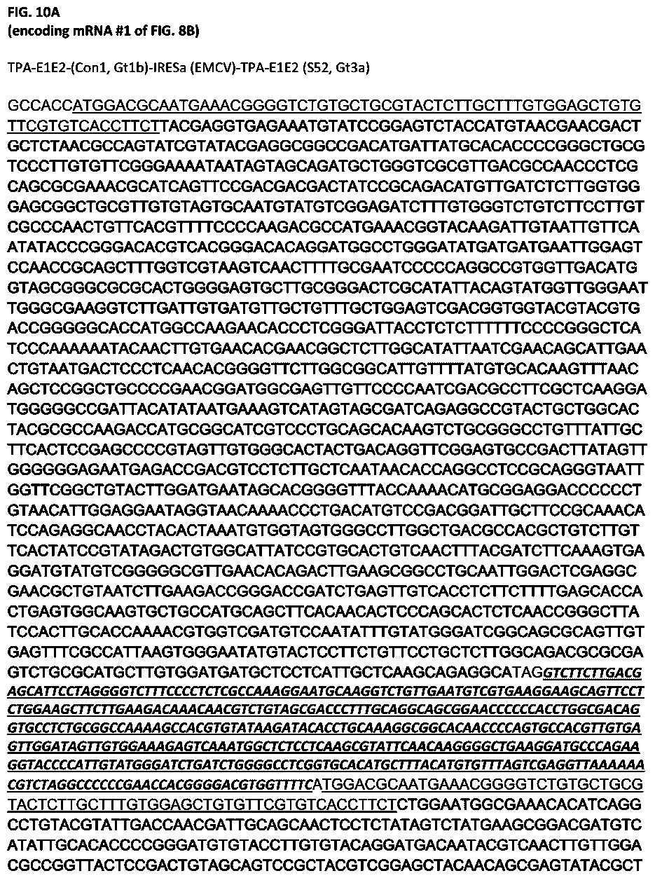

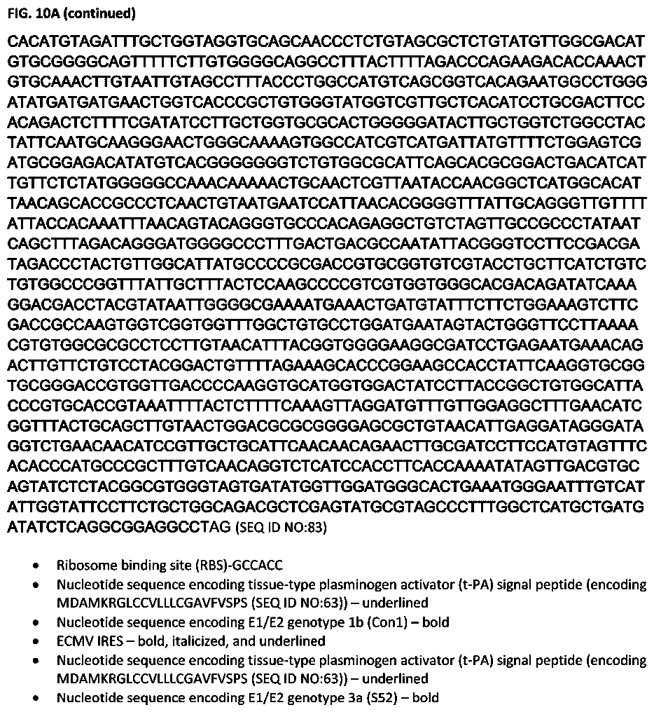

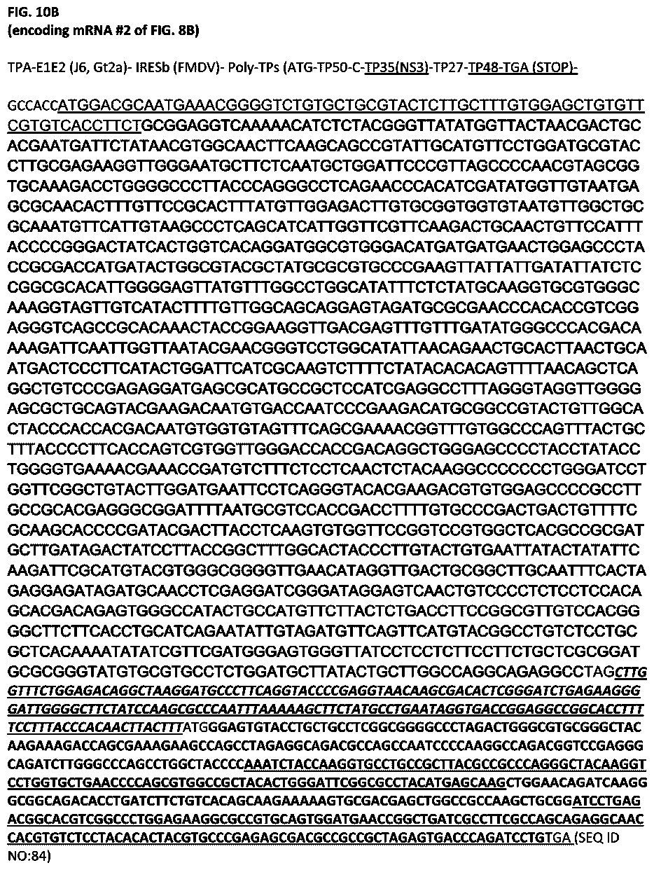

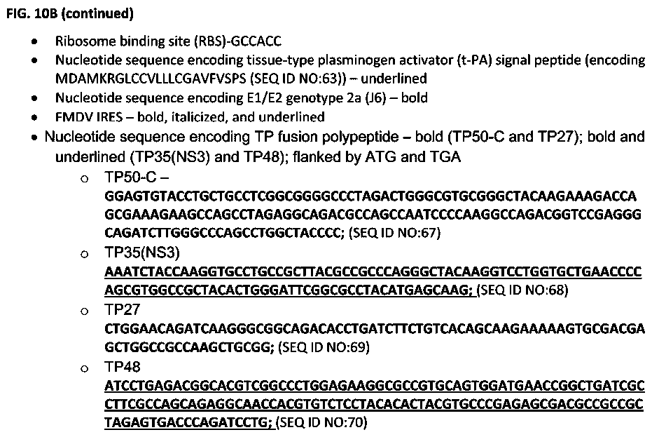

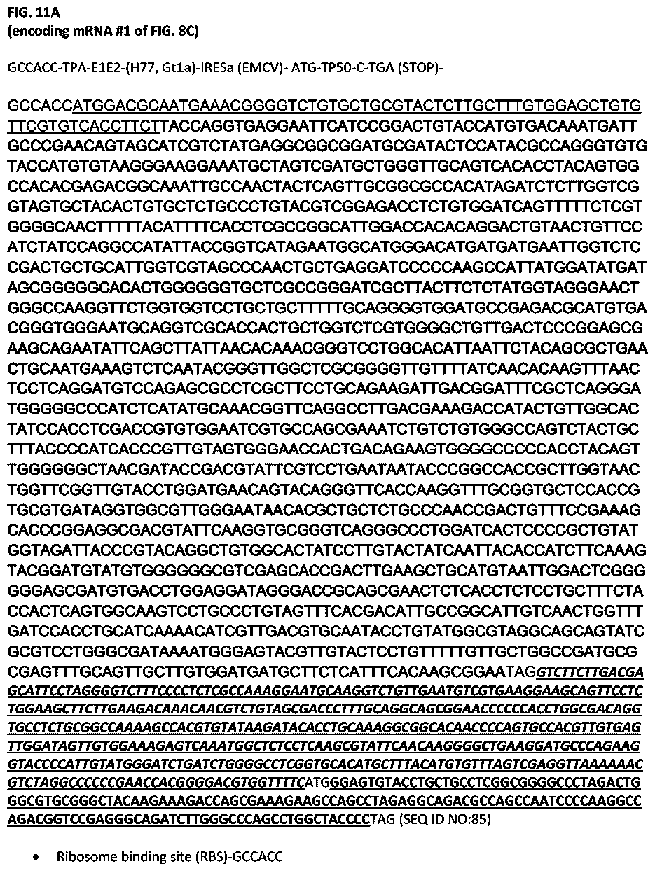

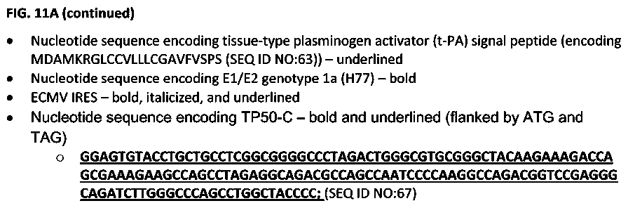

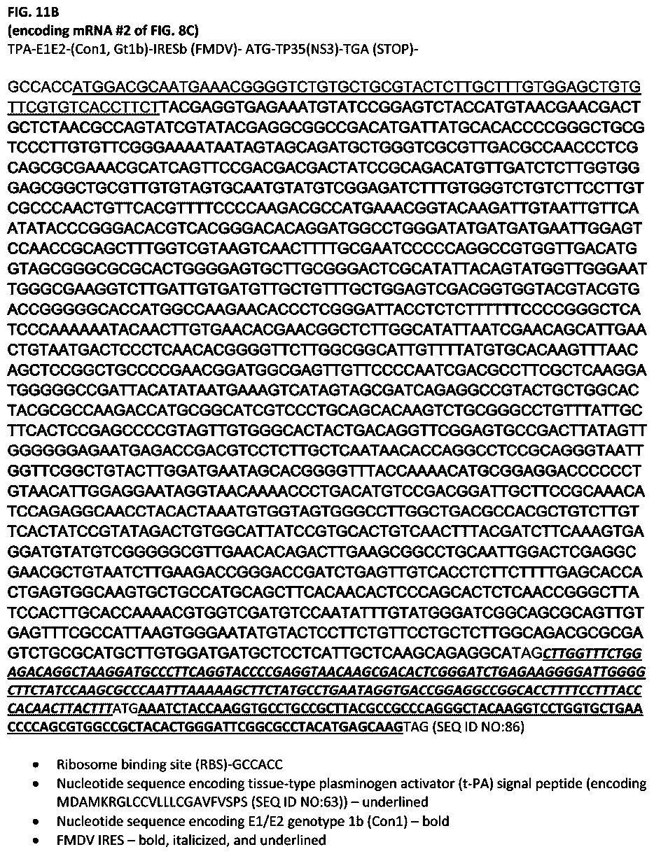

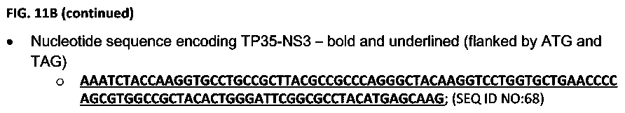

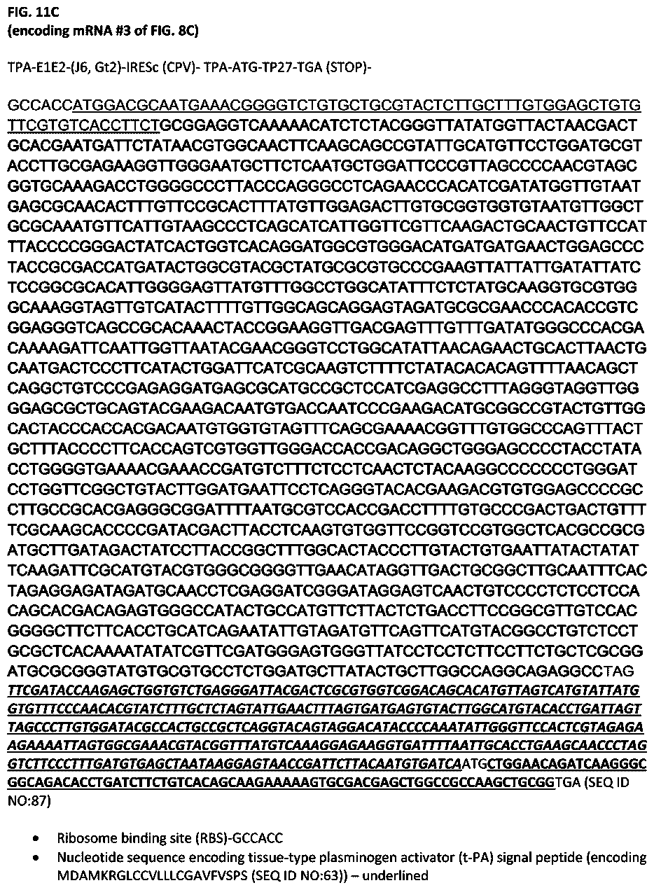

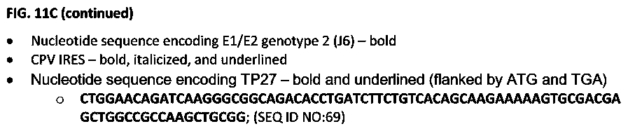

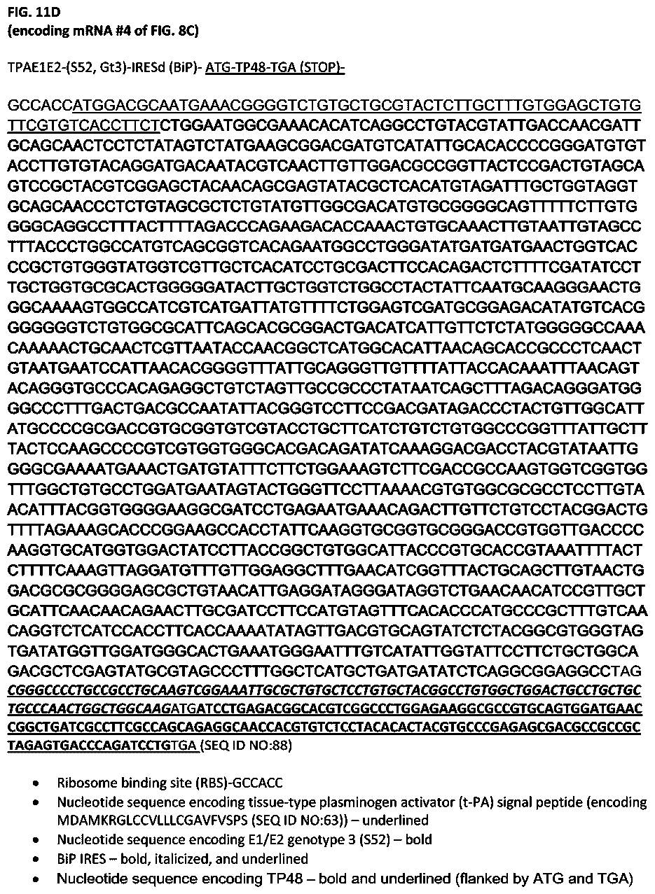

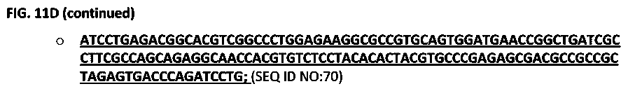

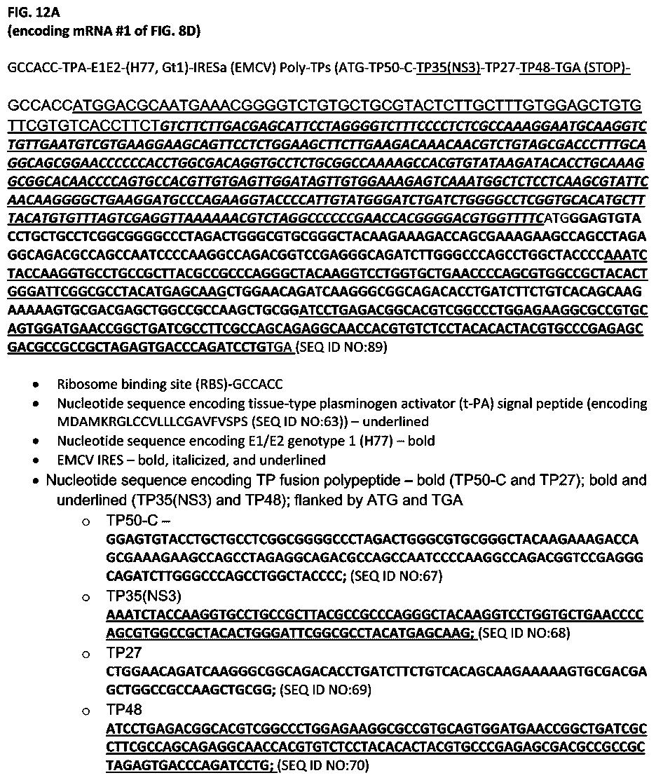

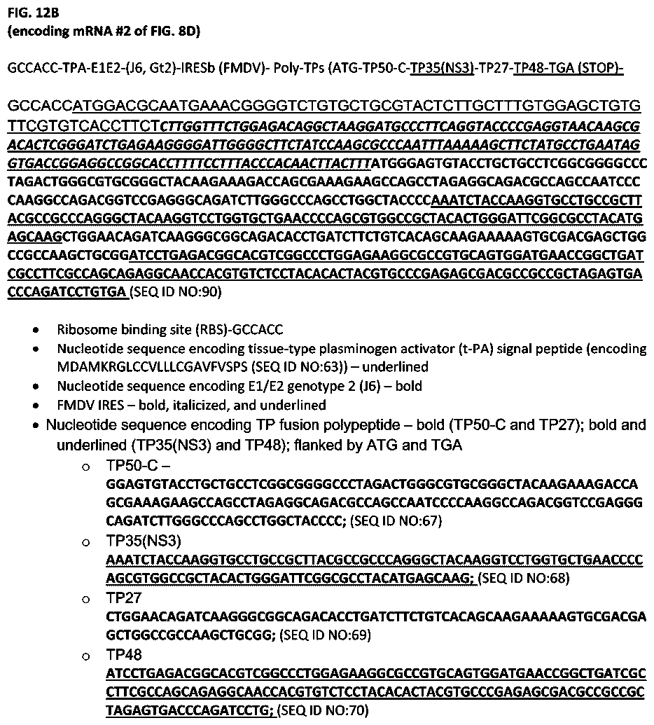

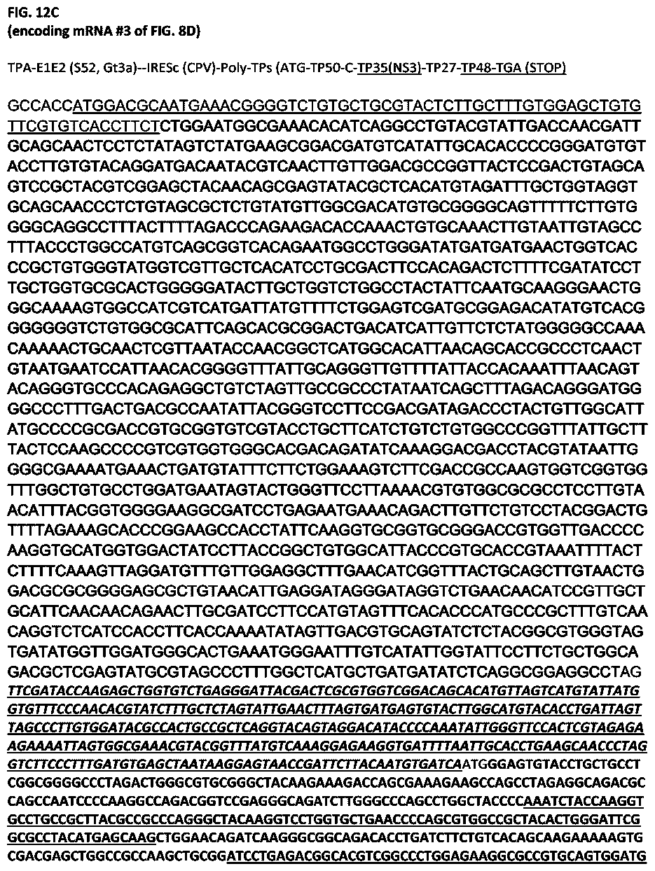

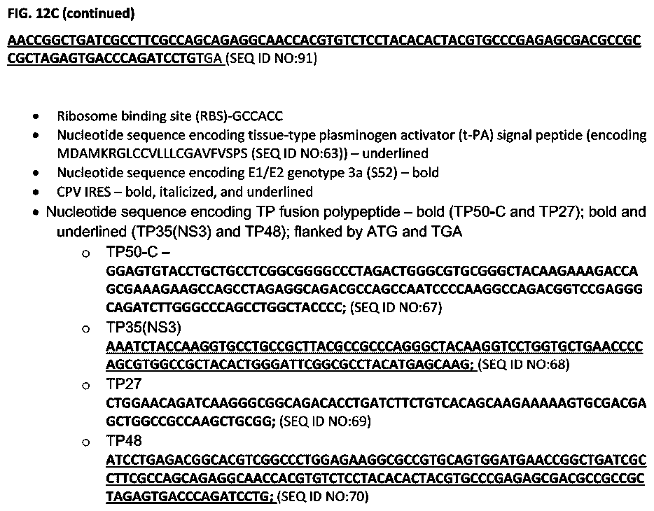

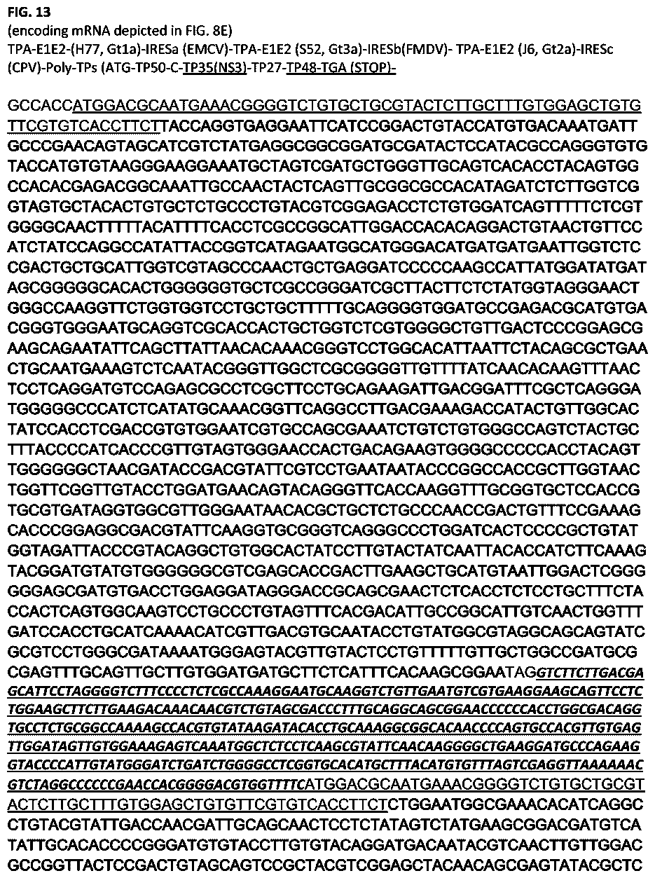

As another example, as illustrated schematically in FIG. 8A, in some cases, a composition of the present disclosure comprises: a) a first RNA molecule comprising: i) a nucleotide sequence encoding HCV E1 and/or E2 polypeptides of genotype 1; ii) an IRES; and iii) one or more nucleotide sequences encoding one or more heterologous polypeptides (e.g., one or more TPs) or a heterologous fusion polypeptide (e.g., a fusion polypeptide comprising two or more (e.g., 1, 2, 3, 4, 5, 6, 7, 8, 9, or 10 TPs); b) a second RNA molecule comprising a nucleotide sequence encoding HCV E1 and E2 polypeptides of genotype 3; and c) a third RNA molecule comprising a nucleotide sequence encoding HCV E1 and E2 polypeptides of genotype 2.

As an example, as depicted in FIG. 9A-9C, in some cases, a composition of the present disclosure comprises: a) a first RNA molecule comprising: i) a nucleotide sequence encoding HCV E1 and HCV E2 polypeptides of genotype 1b and having a heterologous signal peptide (e.g., t-PA); ii) an IRES (e.g., an EMCV IRES); and iii) a nucleotide sequence encoding a fusion polypeptide comprising two or more TPs (e.g., TP50-C; TP35-NS3; TP27; and TP48); b) a second RNA molecule comprising a nucleotide sequence encoding HCV E1 and HCV E2 polypeptides of genotype 3a and having a heterologous signal peptide (e.g., t-PA); and c) a third RNA molecule comprising a nucleotide sequence encoding HCV E1 and HCV E2 polypeptides of genotype 2a and having a heterologous signal peptide (e.g., t-PA). The nucleotide sequences in FIG. 9A-9C can be DNA molecules encoding mRNA molecules, or, where the Ts are replaced with Us, can be mRNA molecules. The DNA molecules can include a polyadenylation signal. The mRNA molecules can include a Cap sequence and can include a poly(A) tail.

As an example, as illustrated schematically in FIG. 8B, in some cases, a composition of the present disclosure comprises: a) a first RNA molecule comprising: i) a nucleotide sequence encoding HCV E1 and/or E2 polypeptides of genotype 1; ii) an IRES; and iii) a nucleotide sequence encoding HCV E1 and/or E2 polypeptides of genotype 3; and b) a second RNA molecule comprising: i) a nucleotide sequence encoding HCV E1 and/or E2 polypeptides of genotype 2; ii) an IRES; and iii) one or more nucleotide sequences encoding one or more heterologous polypeptides (e.g., one or more TPs) or a heterologous fusion polypeptide (e.g., a fusion polypeptide comprising two or more (e.g., 1, 2, 3, 4, 5, 6, 7, 8, 9, or 10 TPs). The IRESs in the first and second RNA molecules can be the same or different.

As an example, as depicted in FIG. 10A-10B, in some cases, a composition of the present disclosure comprises: a) a first RNA molecule comprising: i) a nucleotide sequence encoding HCV E1 and HCV E2 polypeptides of genotype 1b and having a heterologous signal peptide (e.g., t-PA); ii) an IRES (e.g., EMCV IRES); iii) a nucleotide sequence encoding HCV E1 and HCV E2 polypeptides of genotype 3a and having a heterologous signal peptide (e.g., t-PA); and b) a second RNA molecule comprising: i) a nucleotide sequence encoding HCV E1 and HCV E2 polypeptides of genotype 2a and having a heterologous signal peptide (e.g., t-PA); ii) an IRES (e.g., FMDV IRES); and iii) a nucleotide sequence encoding a fusion polypeptide comprising two or more TPs (e.g., TP50-C; TP35-NS3; TP27; and TP48). The nucleotide sequences in FIG. 10A-10B can be DNA molecules encoding mRNA molecules, or, where the Ts are replaced with Us, can be mRNA molecules. The DNA molecules can include a polyadenylation signal. The mRNA molecules can include a Cap sequence and can include a poly(A) tail.

As another example, as depicted schematically in FIG. 8C, in some cases, a composition of the present disclosure comprises: a) a first RNA molecule comprising: i) a nucleotide sequence encoding HCV E1 and/or E2 polypeptides of genotype 1a; ii) an IRES; and iii) a nucleotide sequence encoding a first heterologous polypeptide (e.g., a first TP); b) a second RNA molecule comprising: i) a nucleotide sequence encoding HCV E1 and/or E2 polypeptides of genotype 1b; ii) an IRES; and iii) a nucleotide sequence encoding a second heterologous polypeptide (e.g., a second TP); c) a third RNA molecule comprising: i) a nucleotide sequence encoding HCV E1 and/or E2 polypeptides of genotype 2; ii) an IRES; and iii) a nucleotide sequence encoding a third heterologous polypeptide (e.g., a third TP); and d) a fourth RNA molecule comprising: i) a nucleotide sequence encoding HCV E1 and/or E2 polypeptides of genotype 3; ii) an IRES; and iii) a nucleotide sequence encoding a fourth heterologous polypeptide (e.g., a fourth TP). The IRESs in the first RNA molecule, the second RNA molecule, the third RNA molecule, and the fourth RNA molecule can be the same or different.

As an example, as depicted in FIG. 11A-11D, in some cases, a composition of the present disclosure comprises: a) a first RNA molecule comprising: i) a nucleotide sequence encoding HCV E1 and E2 polypeptides of genotype 1a and having a heterologous signal peptide (e.g., t-PA); ii) an IRES (e.g., EMCV IRES); iii) a nucleotide sequence encoding a signal peptide (e.g., t-PA) and a first TP (e.g., TP50-C); b) a second RNA molecule comprising: i) a nucleotide sequence encoding HCV E1 and E2 polypeptides of genotype 1b and having a heterologous signal peptide (e.g., t-PA); ii) an IRES (e.g., FMDV IRES); and iii) a nucleotide sequence encoding a signal peptide (e.g., t-PA) and a second TP (e.g., TP35-NS3; c) a third RNA molecule comprising: i) a nucleotide sequence encoding HCV E1 and E2 polypeptides of genotype 2 and having a heterologous signal peptide (e.g., t-PA); ii) an IRES (e.g., CPV IRES); and iii) a nucleotide sequence encoding a signal peptide (e.g., t-PA) and a third TP (e.g., TP27; and d) a fourth RNA molecule comprising: i) a nucleotide sequence encoding HCV E1 and E2 polypeptides of genotype 3 and having a heterologous signal peptide (e.g., t-PA); ii) an IRES (e.g., BiP IRES); and iii) a nucleotide sequence encoding a signal peptide (e.g., t-PA) and a fourth TP (e.g., TP48). The nucleotide sequences in FIG. 11A-11D can be DNA molecules encoding mRNA molecules, or, where the Ts are replaced with Us, can be mRNA molecules. The DNA molecules can include a polyadenylation signal. The mRNA molecules can include a Cap sequence and can include a poly(A) tail.

As another example, in some cases, a composition of the present disclosure is a variation of the embodiment depicted schematically in FIG. 8C, in which the variation includes only 3 RNA molecules; for example, in some cases, a composition of the present disclosure comprises: a) a first RNA molecule comprising: i) a nucleotide sequence encoding HCV E1 and/or E2 polypeptides of genotype 1a; ii) an IRES; and iii) a nucleotide sequence encoding a first heterologous polypeptide (e.g., a first TP); b) a second RNA molecule comprising: i) a nucleotide sequence encoding HCV E1 and/or E2 polypeptides of genotype 3a; ii) an IRES; and iii) a nucleotide sequence encoding a second heterologous polypeptide (e.g., a second TP); and c) a third RNA molecule comprising: i) a nucleotide sequence encoding HCV E1 and/or E2 polypeptides of genotype 2a; ii) an IRES; and iii) a nucleotide sequence encoding a third heterologous polypeptide (e.g., a third TP). The IRESs in the first RNA molecule, the second RNA molecule, and the third RNA molecule can be the same or different. As one non-limiting example, in some cases, the first TP is a TP228 polypeptide, the second TP is a TP50C polypeptide, and the third TP is a TP48 polypeptide. As another non-limiting example, in some cases, the first TP is a TP240 polypeptide, the second TP is a TP50C polypeptide, and the third TP is a TP48 polypeptide. For example, the TP48 polypeptide comprises an amino acid sequence having at least 90%, at least 95%, at least 98%, at least 99%, or 100%, amino acid sequence identity to the following amino acid sequence: ILRRHVGPGEGAVQWMNRLIAFASRGNHVSPTHYVPESDASARVTQIL (SEQ ID NO:338); and has a length of 48 amino acids. The TP50C polypeptide can comprise an amino acid sequence having at least 90%, at least 95%, at least 98%, at least 99%, or 100%, amino acid sequence identity to the following amino acid sequence: GVYLLPRRGPRLGVRATRKTSERSQPRGRRQPIPKARRSEGRSWAQPGYP (SEQ ID NO:101); and can have a length of 50 amino acids. The TP228 polypeptide can comprise an amino acid sequence having at least 90%, at least 95%, at least 98%, at least 99%, or 100%, amino acid sequence identity to the TP228 amino acid sequence depicted in FIG. 4; and can have a length of about 228 amino acids. For example, TP228 polypeptide can comprise an amino acid sequence having at least 90%, at least 95%, at least 98%, at least 99%, or 100%, amino acid sequence identity to the following TP228 amino acid sequence: LHAPTGSGKSTKVPAAYAAQGYKVLVLNPSVAATLGFGAYMSKAHGIDPNIRTGVRTITTGSPI TYSTYGKFLADGGCSGGAYDIIICDECHSTDATSILGIGTVLDQAETAGARLVVLATATPPGSVT VPHPNIEEVALSTTGEIPFYGKAIPLEVIKGGRHLIFCHSKKKCDELAAKLVALGINAVAYYRGL DVSVIPTSGDVVVVATDALMTGFTGDFDSVIDCN (SEQ ID NO:382); and can have a length of 228 amino acids. In some cases, a TP240 polypeptide comprises an amino acid sequence having at least 90%, at least 95%, at least 98%, at least 99%, or 100%, amino acid sequence identity to the following TP240 amino acid sequence: SYQVGYLHAPTGSGKSTKVPAAYAAQGYKVLVLNPSVAATLGFGAYMSKAHGIDPNIRTGVRT VTTGAPITYSTYGKFLADGGCSGGAYDIIICDECHSTDATTILGIGTVLDQAETAGARLVVLATA TPPGSVTVPHPNIEEVALGTEGEIPFYGKAIPLEVIKGGRHLIFCHSKKKCDELAAKLRGMGLNA VAYYRGLDVSVIPTSGDVVVVATDALMTGYTGDFDSVIDCNVAVTQT (SEQ ID NO:380), and has a length of 240 amino acids. In some cases, a TP240 polypeptide comprises an amino acid sequence having at least 90%, at least 95%, at least 98%, at least 99%, or 100%, amino acid sequence identity to the following TP240 amino acid sequence: SYQVGYLHAPTGSGKSTKVPAAYAAQGYKVLVLNPSVAATLGFGAYMSKAHGIDPNIRTGVRT VTTGAPITYSTYGKFLADGGCSGGAYDIIICDECHSVDATTILGIGTVLDQAETAGARLVVLATA TPPGSVTVPHPNIEEVALGNEGEIPFYGKAIPLSTIKGGRHLIFCHSKKKCDELAAKLRGMGLNA VAYYRGLDVSVIPTQGDVVVVATDALMTGYTGDFDSVIDCNVAVTQT (SEQ ID NO:381), and has a length of 240 amino acids. In some cases, HCV E1 and E2 polypeptides of genotype 1a have amino acid sequences having at least 90%, at least 95%, at least 98%, at least 99%, or 100%, amino acid sequence identity to the H77 (NP_671491) E1 and E2 amino acid sequences depicted in FIG. 1A-1C (e.g., where amino acids 192-383 of FIG. 1A-IC are HCV E1; and amino acids 384-747 of FIG. 1A-IC are HCV E2). In some cases, HCV E1 and E2 polypeptides of genotype 2a have amino acid sequences having at least 90%, at least 95%, at least 98%, at least 99%, or 100%, amino acid sequence identity to the J6 (D00944 (HC-J6)) E1 and E2 amino acid sequences depicted in FIG. 2A-2C (e.g., where amino acids 192-384 of FIG. 2A-2C are HCV E1; and amino acids 385-751 of FIG. 2A-2C are HCV E2). In some cases, HCV E1 and E2 polypeptides of genotype 3 have amino acid sequences having at least 90%, at least 95%, at least 98%, at least 99%, or 100%, amino acid sequence identity to the S52 (ADF97232) E1 and E2 amino acid sequences depicted in FIG. 3A-3C (e.g., where amino acids 192-384 of FIG. 3A-3C are HCV E1; and amino acids 385-754 of FIG. 3A-3C are HDV E2).

As another example, as illustrated schematically in FIG. 8D, in some cases, a composition of the present disclosure comprises: a) a first RNA molecule comprising: i) a nucleotide sequence encoding HCV E1 and/or E2 polypeptides of genotype 1; ii) an IRES; and iii) one or more nucleotide sequences encoding one or more heterologous polypeptides (e.g., one or more TPs) or a heterologous fusion polypeptide (e.g., a fusion polypeptide comprising two or more (e.g., 1, 2, 3, 4, 5, 6, 7, 8, 9, or 10 TPs); b) a second RNA molecule comprising: i) a nucleotide sequence encoding HCV E1 and/or E2 polypeptides of genotype 2; ii) an IRES; and iii) one or more nucleotide sequences encoding one or more heterologous polypeptides (e.g., one or more TPs) or a heterologous fusion polypeptide (e.g., a fusion polypeptide comprising two or more (e.g., 1, 2, 3, 4, 5, 6, 7, 8, 9, or 10 TPs); and c) a third RNA molecule comprising: i) a nucleotide sequence encoding HCV E1 and/or E2 polypeptides of genotype 3; ii) an IRES; and iii) one or more nucleotide sequences encoding one or more heterologous polypeptides (e.g., one or more TPs) or a heterologous fusion polypeptide (e.g., a fusion polypeptide comprising two or more (e.g., 1, 2, 3, 4, 5, 6, 7, 8, 9, or 10 TPs). The IRESs in the first RNA molecule, the second RNA molecule, and the third RNA molecule can be the same or different.

As an example, as depicted in FIG. 12A-12C, in some cases, a composition of the present disclosure comprises: a) a first RNA molecule comprising: i) a nucleotide sequence encoding HCV E1 and E2 polypeptides of genotype 1 and having a heterologous signal peptide (e.g., t-PA); ii) an IRES (e.g., EMCV IRES); and iii) a nucleotide sequence encoding a TP fusion polypeptide comprising TP50-C; TP35-NS3; TP27; and TP48; b) a second RNA molecule comprising: i) a nucleotide sequence encoding HCV E1 and E2 polypeptides of genotype 2 and having a heterologous signal peptide (e.g., t-PA); ii) an IRES (e.g., FMDV IRES); and iii) a nucleotide sequence encoding a TP fusion polypeptide comprising TP50-C; TP35-NS3; TP27; and TP48; and c) a third RNA molecule comprising: i) a nucleotide sequence encoding HCV E1 and E2 polypeptides of genotype 3a and having a heterologous signal peptide (e.g., t-PA); ii) an IRES (e.g., CPV IRES); and iii) a nucleotide sequence encoding a TP fusion polypeptide comprising TP50-C; TP35-NS3; TP27; and TP48. The nucleotide sequences in FIG. 12A-12C can be DNA molecules encoding mRNA molecules, or, where the Ts are replaced with Us, can be mRNA molecules. The DNA molecules can include a polyadenylation signal. The mRNA molecules can include a Cap sequence and can include a poly(A) tail.

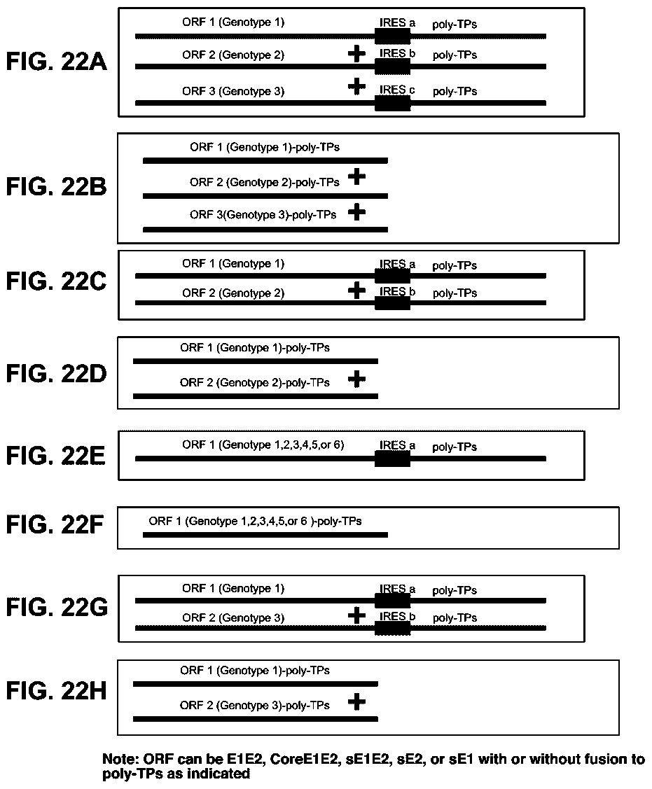

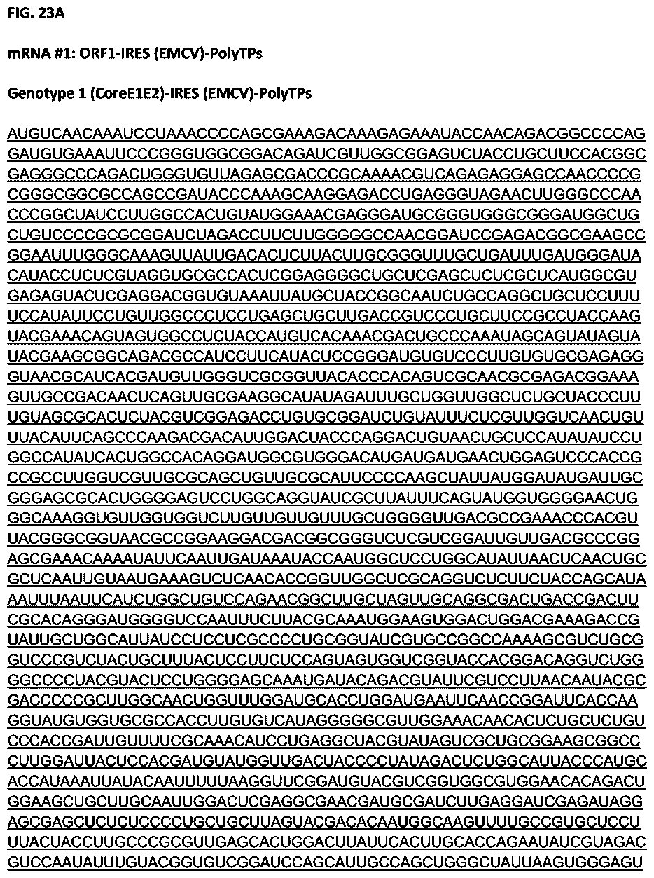

As another example, as depicted schematically in FIG. 22A, in some cases, a composition of the present disclosure comprises: a) a first RNA molecule comprising: i) a nucleotide sequence encoding one or more HCV polypeptides (e.g., an HCV E1 polypeptide; an HCV E2 polypeptide; a soluble HCV E1 (sE1) polypeptide; a soluble HCV E2 (sE2) polypeptide; an HCV E1/E2 heterodimer; a soluble E1/E2 heterodimer (sE1/E2); or a Core-E1-E2 polyprotein) of genotype 1, and optionally including a heterologous signal peptide (e.g., t-PA); ii) an IRES (e.g., EMCV IRES); and iii) a nucleotide sequence encoding a TP fusion polypeptide comprising TP50-C; TP35-NS3; TP27; and TP48; b) a second RNA molecule comprising: i) a nucleotide sequence encoding one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 2, and optionally including a heterologous signal peptide (e.g., t-PA); ii) an IRES (e.g., FMDV IRES); and iii) a nucleotide sequence encoding a TP fusion polypeptide comprising TP50-C; TP35-NS3; TP27; and TP48; and c) a third RNA molecule comprising: i) a nucleotide sequence encoding one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 3, and optionally including a heterologous signal peptide (e.g., t-PA); ii) an IRES (e.g., CPV IRES); and iii) a nucleotide sequence encoding a TP fusion polypeptide comprising TP50-C; TP35-NS3; TP27; and TP48. The mRNA molecules can include a Cap sequence and can include a poly(A) tail. In some cases, the genotype 1 E1 and/or the E2 polypeptides are H77 polypeptides, as described above. In some cases, the genotype 1 E1 and/or the E2 polypeptides are soluble H77 polypeptides, as described above. In some cases, the genotype 2 E1 and/or the E2 polypeptides are J6 polypeptides, as described above. In some cases, the genotype 2 E1 and/or the E2 polypeptides are soluble J6 polypeptides, as described above. In some cases, the genotype 3 E1 and/or the E2 polypeptides are S52 polypeptides, as described above. In some cases, the genotype 3 E1 and/or the E2 polypeptides are soluble S52 polypeptides, as described above.

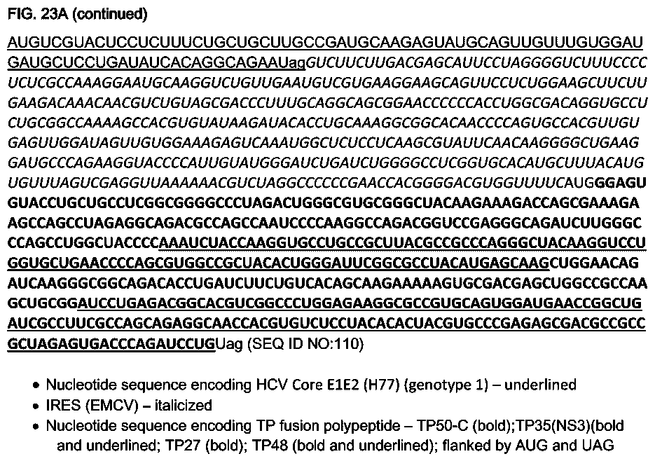

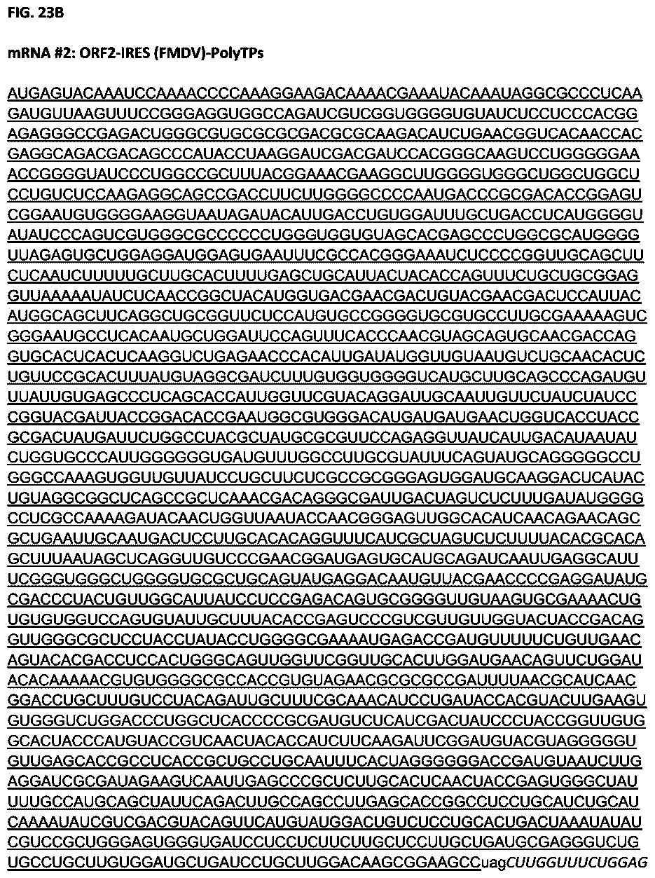

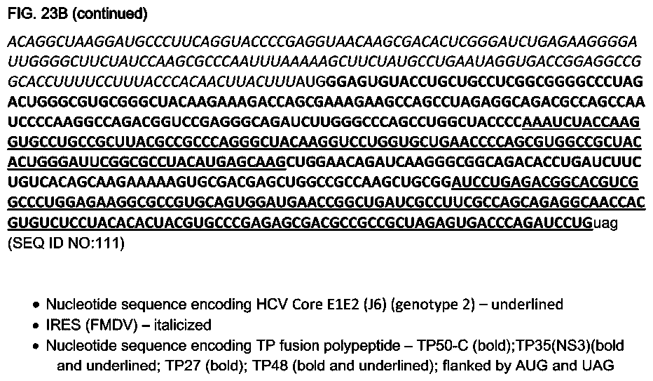

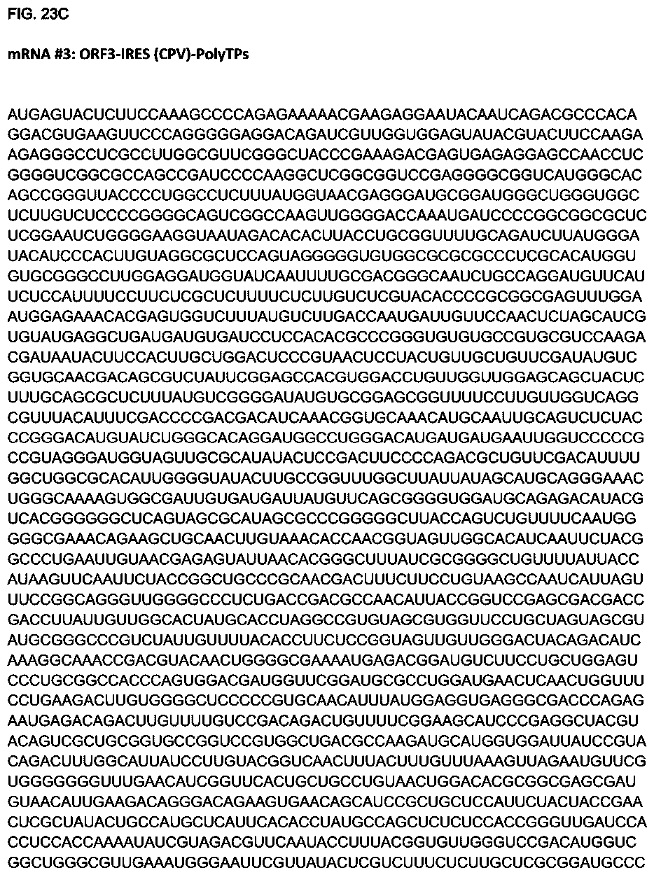

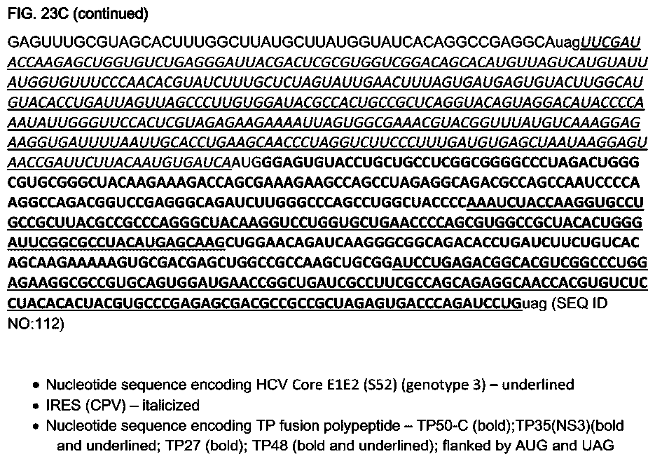

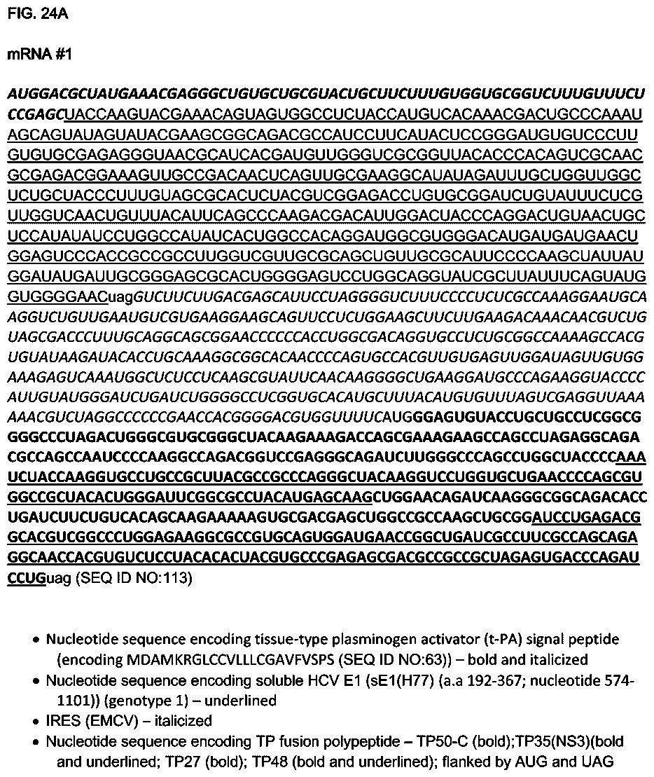

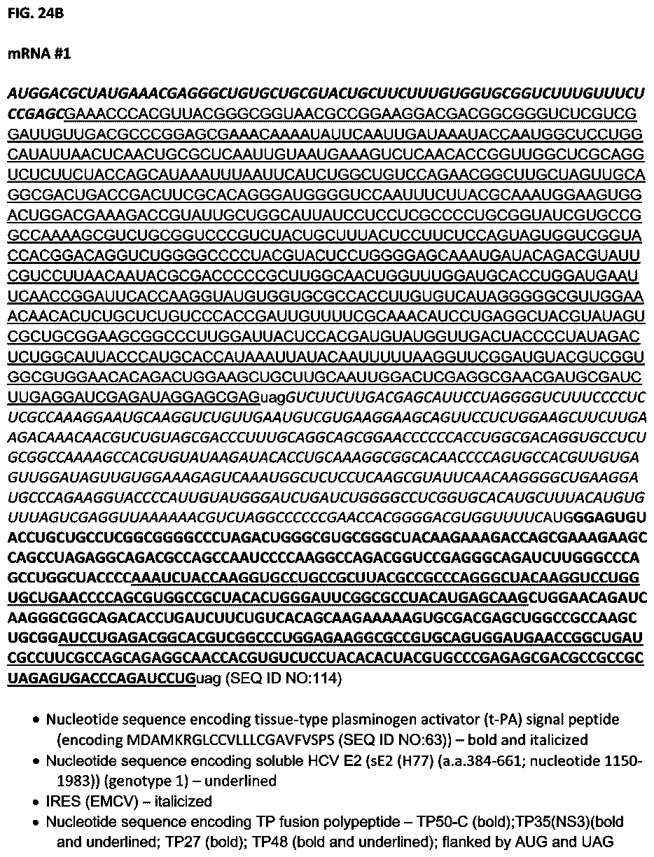

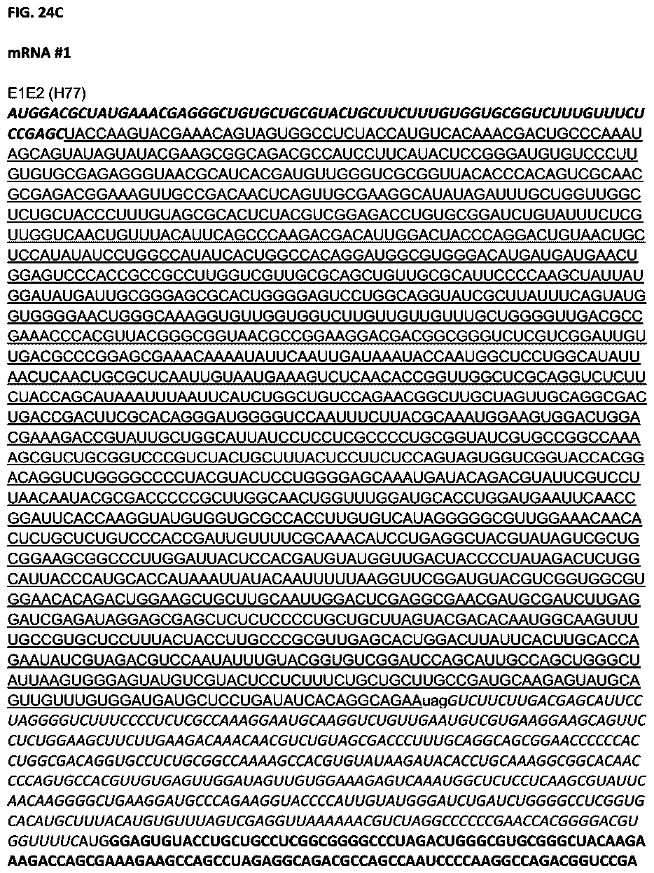

Examples of nucleotide sequences of mRNAs depicted schematically in FIG. 22A are provided in FIG. 23A-23C and FIG. 24A-24I. For example, as shown in FIG. 23A-23C, in some cases, a composition of the present disclosure comprises: a) a first RNA molecule (FIG. 23A) comprising: i) a nucleotide sequence encoding an HCV Core-E1-E2 polyprotein of genotype 1; ii) an FMDV IRES; and ii) a nucleotide sequence encoding a TP fusion polypeptide comprising TP50-C; TP35-NS3; TP27; and TP48; b) a second RNA molecule (FIG. 23B) comprising: i) a nucleotide sequence encoding an HCV Core-E1-E2 polyprotein of genotype 2; ii) an FMDV IRES; and ii) a nucleotide sequence encoding a TP fusion polypeptide comprising TP50-C; TP35-NS3; TP27; and TP48; and c) a third RNA molecule (FIG. 23A) comprising: i) a nucleotide sequence encoding an HCV Core-E1-E2 polyprotein of genotype 3; ii) an FMDV IRES; and ii) a nucleotide sequence encoding a TP fusion polypeptide comprising TP50-C; TP35-NS3; TP27; and TP48. In some cases, the first RNA molecule comprises, instead of a nucleotide sequence encoding an HCV Core-E1-E2 polyprotein of genotype 1, a nucleotide sequence encoding a soluble HCV E1 polypeptide of genotype 1. As an example, in some cases, the first RNA molecule comprises the nucleotide sequence depicted in FIG. 24A. In some cases, the first RNA molecule comprises, instead of a nucleotide sequence encoding an HCV Core-E1-E2 polyprotein of genotype 1, a nucleotide sequence encoding a soluble HCV E2 polypeptide of genotype 1. As an example, in some cases, the first RNA molecule comprises the nucleotide sequence depicted in FIG. 24B. In some cases, the first RNA molecule comprises, instead of a nucleotide sequence encoding an HCV Core-E1-E2 polyprotein of genotype 1, a nucleotide sequence encoding an HCV E1/E2 heterodimer of genotype 1. As an example, in some cases, the first RNA molecule comprises the nucleotide sequence depicted in FIG. 24C. In some cases, the second RNA molecule comprises, instead of a nucleotide sequence encoding an HCV Core-E1-E2 polyprotein of genotype 2, a nucleotide sequence encoding a soluble HCV E1 polypeptide of genotype 1. As an example, in some cases, the second RNA molecule comprises the nucleotide sequence depicted in FIG. 24D. In some cases, the second RNA molecule comprises, instead of a nucleotide sequence encoding an HCV Core-E1-E2 polyprotein of genotype 2, a nucleotide sequence encoding a soluble HCV E2 polypeptide of genotype 2. As an example, in some cases, the second RNA molecule comprises the nucleotide sequence depicted in FIG. 24E. In some cases, the second RNA molecule comprises, instead of a nucleotide sequence encoding an HCV Core-E1-E2 polyprotein of genotype 2, a nucleotide sequence encoding an HCV E1/E2 heterodimer of genotype 2. As an example, in some cases, the second RNA molecule comprises the nucleotide sequence depicted in FIG. 24F. In some cases, the third RNA molecule comprises, instead of a nucleotide sequence encoding an HCV Core-E1-E2 polyprotein of genotype 3, a nucleotide sequence encoding a soluble HCV E1 polypeptide of genotype 3. As an example, in some cases, the third RNA molecule comprises the nucleotide sequence depicted in FIG. 24G. In some cases, the third RNA molecule comprises, instead of a nucleotide sequence encoding an HCV Core-E1-E2 polyprotein of genotype 3, a nucleotide sequence encoding a soluble HCV E2 polypeptide of genotype 3. As an example, in some cases, the third RNA molecule comprises the nucleotide sequence depicted in FIG. 24H. In some cases, the third RNA molecule comprises, instead of a nucleotide sequence encoding an HCV Core-E1-E2 polyprotein of genotype 3, a nucleotide sequence encoding an HCV E1/E2 heterodimer of genotype 3. As an example, in some cases, the third RNA molecule comprises the nucleotide sequence depicted in FIG. 241.

In some cases, a composition of the present disclosure comprises: a) a first RNA molecule, where the first RNA molecule comprises the nucleotide sequence depicted in any one of FIG. 23A, FIG. 24A, FIG. 24B, and FIG. 24C; b) a second RNA molecule, where the second RNA molecule comprises the nucleotide sequence depicted in any one of FIG. 23B, FIG. 24D, FIG. 24E, and FIG. 24F; and c) a third RNA molecule, where the third RNA molecule comprises the nucleotide sequence depicted in any one of FIG. 23C, FIG. 24G, FIG. 24H, and FIG. 241.

As another example, as depicted schematically in FIG. 22B, in some cases, a composition of the present disclosure comprises: a) a first RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 1; and ii) a TP fusion polypeptide comprising TP50-C; TP35-NS3; TP27; and TP48; b) a second RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 2; and ii) a TP fusion polypeptide comprising TP50-C; TP35-NS3; TP27; and TP48; and c) a third RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 3; and ii) a TP fusion polypeptide comprising TP50-C; TP35-NS3; TP27; and TP48. The mRNA molecules can include a Cap sequence and can include a poly(A) tail. In some cases, the genotype 1 E1 and/or the E2 polypeptides are H77 polypeptides, as described above. In some cases, the genotype 1 E1 and/or the E2 polypeptides are soluble H77 polypeptides, as described above. In some cases, the genotype 2 E1 and/or the E2 polypeptides are J6 polypeptides, as described above. In some cases, the genotype 2 E1 and/or the E2 polypeptides are soluble J6 polypeptides, as described above. In some cases, the genotype 3 E1 and/or the E2 polypeptides are S52 polypeptides, as described above. In some cases, the genotype 3 E1 and/or the E2 polypeptides are soluble S52 polypeptides, as described above.

Examples of nucleotide sequences of mRNAs depicted schematically in FIG. 22B are provided in FIG. 25A-25L. For example, as shown in FIG. 25A-25C, in some cases, a composition of the present disclosure comprises: a) a first RNA molecule (FIG. 25A) comprising a nucleotide sequence encoding a fusion polypeptide comprising: i) a HCV Core-E1-E2 polyprotein of genotype 1; and ii) a TP fusion polypeptide comprising TP50-C; TP35-NS3; TP27; and TP48; b) a second RNA molecule (FIG. 25B) comprising a nucleotide sequence encoding a fusion polypeptide comprising: i) a HCV Core-E1-E2 polyprotein of genotype 2; and ii) a TP fusion polypeptide comprising TP50-C; TP35-NS3; TP27; and TP48 and c) a third RNA molecule (FIG. 25C) comprising a nucleotide sequence encoding a fusion polypeptide comprising: i) a HCV Core-E1-E2 polyprotein of genotype 3; and ii) a TP fusion polypeptide comprising TP50-C; TP35-NS3; TP27; and TP48.

In some cases, the first RNA molecule comprises, instead of a nucleotide sequence encoding an HCV Core-E1-E2 polyprotein of genotype 1, a nucleotide sequence encoding a soluble HCV E1 polypeptide of genotype 1. As an example, in some cases, the first RNA molecule comprises the nucleotide sequence depicted in FIG. 25D. In some cases, the first RNA molecule comprises, instead of a nucleotide sequence encoding an HCV Core-E1-E2 polyprotein of genotype 1, a nucleotide sequence encoding a soluble HCV E2 polypeptide of genotype 1. As an example, in some cases, the first RNA molecule comprises the nucleotide sequence depicted in FIG. 25E. In some cases, the first RNA molecule comprises, instead of a nucleotide sequence encoding an HCV Core-E1-E2 polyprotein of genotype 1, a nucleotide sequence encoding an HCV E1/E2 heterodimer of genotype 1. As an example, in some cases, the first RNA molecule comprises the nucleotide sequence depicted in FIG. 25F. In some cases, the second RNA molecule comprises, instead of a nucleotide sequence encoding an HCV Core-E1-E2 polyprotein of genotype 2, a nucleotide sequence encoding a soluble HCV E1 polypeptide of genotype 2. As an example, in some cases, the second RNA molecule comprises the nucleotide sequence depicted in FIG. 25G. In some cases, the second RNA molecule comprises, instead of a nucleotide sequence encoding an HCV Core-E1-E2 polyprotein of genotype 2, a nucleotide sequence encoding a soluble HCV E2 polypeptide of genotype 2. As an example, in some cases, the second RNA molecule comprises the nucleotide sequence depicted in FIG. 25H. In some cases, the third RNA comprises, instead of a nucleotide sequence encoding an HCV Core-E1-E2 polyprotein of genotype 3, a nucleotide sequence encoding a soluble HCV E1 polypeptide of genotype 3. As an example, in some cases, the third RNA molecule comprises the nucleotide sequence depicted in FIG. 25J. In some cases, the third RNA comprises, instead of a nucleotide sequence encoding an HCV Core-E1-E2 polyprotein of genotype 3, a nucleotide sequence encoding a soluble HCV E2 polypeptide of genotype 3. As an example, in some cases, the third RNA molecule comprises the nucleotide sequence depicted in FIG. 25K. In some cases, the third RNA comprises, instead of a nucleotide sequence encoding an HCV Core-E1-E2 polyprotein of genotype 3, a nucleotide sequence encoding an HCV E1/E2 heterodimer of genotype 3. As an example, in some cases, the third RNA molecule comprises the nucleotide sequence depicted in FIG. 25L.

In some cases, a composition of the present disclosure comprises: a) a first RNA molecule, where the first RNA molecule comprises the nucleotide sequence depicted in any one of FIG. 25A, FIG. 25D, FIG. 25E, and FIG. 25F; b) a second RNA molecule, where the second RNA molecule comprises the nucleotide sequence depicted in any one of FIG. 25B, FIG. 25G, FIG. 25H, and FIG. 251; and c) a third RNA molecule, where the third molecule comprises the nucleotide sequence depicted in any one of FIG. 25C, FIG. 25J, FIG. 25K, and FIG. 25L.

In some cases, a composition of the present disclosure comprises only two types of RNA molecules. In some cases, a composition of the present disclosure comprises: a) a first RNA molecule comprising: i) a nucleotide sequence encoding one or more HCV polypeptides (e.g., an HCV E1 polypeptide; an HCV E2 polypeptide; a soluble HCV E1 (sE1) polypeptide; a soluble HCV E2 (sE2) polypeptide; an HCV E1/E2 heterodimer; a soluble E1/E2 heterodimer (sE1/E2); or a Core-E1-E2 polyprotein) of a first genotype, and optionally including a heterologous signal peptide (e.g., t-PA); ii) an IRES (e.g., EMCV IRES); and iii) a nucleotide sequence encoding a TP fusion polypeptide comprising TP50-C; TP35-NS3; TP27; and TP48; and b) a second RNA molecule comprising: i) a nucleotide sequence encoding one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of a second genotype, and optionally including a heterologous signal peptide (e.g., t-PA); ii) an IRES (e.g., FMDV IRES); and iii) a nucleotide sequence encoding a TP fusion polypeptide comprising TP50-C; TP35-NS3; TP27; and TP48. The mRNA molecules can include a Cap sequence and can include a poly(A) tail. For example, as depicted schematically in FIG. 22C, a composition of the present disclosure comprises: a) a first RNA molecule comprising: i) a nucleotide sequence encoding one or more HCV polypeptides (e.g., an HCV E1 polypeptide; an HCV E2 polypeptide; a soluble HCV E1 (sE1) polypeptide; a soluble HCV E2 (sE2) polypeptide; an HCV E1/E2 heterodimer; a soluble E1/E2 heterodimer (sE1/E2); or a Core-E1-E2 polyprotein) of genotype 1, and optionally including a heterologous signal peptide (e.g., t-PA); ii) an IRES (e.g., EMCV IRES); and iii) a nucleotide sequence encoding a TP fusion polypeptide comprising TP50-C; TP35-NS3; TP27; and TP48; and b) a second RNA molecule comprising: i) a nucleotide sequence encoding one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 2, and optionally including a heterologous signal peptide (e.g., t-PA); ii) an IRES (e.g., FMDV IRES); and iii) a nucleotide sequence encoding a TP fusion polypeptide comprising TP50-C; TP35-NS3; TP27; and TP48. As another example, as depicted schematically in FIG. 22G, a composition of the present disclosure comprises: a) a first RNA molecule comprising: i) a nucleotide sequence encoding one or more HCV polypeptides (e.g., an HCV E1 polypeptide; an HCV E2 polypeptide; a soluble HCV E1 (sE1) polypeptide; a soluble HCV E2 (sE2) polypeptide; an HCV E1/E2 heterodimer; a soluble E1/E2 heterodimer (sE1/E2); or a Core-E1-E2 polyprotein) of genotype 1, and optionally including a heterologous signal peptide (e.g., t-PA); ii) an IRES (e.g., EMCV IRES); and iii) a nucleotide sequence encoding a TP fusion polypeptide comprising TP50-C; TP35-NS3; TP27; and TP48; and b) a second RNA molecule comprising: i) a nucleotide sequence encoding one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 3, and optionally including a heterologous signal peptide (e.g., t-PA); ii) an IRES (e.g., FMDV IRES); and iii) a nucleotide sequence encoding a TP fusion polypeptide comprising TP50-C; TP35-NS3; TP27; and TP48. The mRNA molecules can include a Cap sequence and can include a poly(A) tail. In some cases, the genotype 1 E1 and/or the E2 polypeptides are H77 polypeptides, as described above. In some cases, the genotype 1 E1 and/or the E2 polypeptides are soluble H77 polypeptides, as described above. In some cases, the genotype 3 E1 and/or the E2 polypeptides are S52 polypeptides, as described above. In some cases, the genotype 3 E1 and/or the E2 polypeptides are soluble S52 polypeptides, as described above.

As another example, in some cases, a composition of the present disclosure comprises: a) a first RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of a first genotype; and ii) a TP fusion polypeptide comprising TP50-C; TP35-NS3; TP27; and TP48; and b) a second RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of a second genotype; and ii) a TP fusion polypeptide comprising TP50-C; TP35-NS3; TP27; and TP48. The mRNA molecules can include a Cap sequence and can include a poly(A) tail. As an example, as depicted schematically in FIG. 22D, in some cases, a composition of the present disclosure comprises: a) a first RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 1; and ii) a TP fusion polypeptide comprising TP50-C; TP35-NS3; TP27; and TP48; and b) a second RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 2; and ii) a TP fusion polypeptide comprising TP50-C; TP35-NS3; TP27; and TP48. As another example, as depicted schematically in FIG. 22H, in some cases, a composition of the present disclosure comprises: a) a first RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 1; and ii) a TP fusion polypeptide comprising TP50-C; TP35-NS3; TP27; and TP48; and b) a second RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 3; and ii) a TP fusion polypeptide comprising TP50-C; TP35-NS3; TP27; and TP48. The mRNA molecules can include a Cap sequence and can include a poly(A) tail. In some cases, the genotype 1 E1 and/or the E2 polypeptides are H77 polypeptides, as described above. In some cases, the genotype 1 E1 and/or the E2 polypeptides are soluble H77 polypeptides, as described above. In some cases, the genotype 3 E1 and/or the E2 polypeptides are S52 polypeptides, as described above. In some cases, the genotype 3 E1 and/or the E2 polypeptides are soluble S52 polypeptides, as described above.

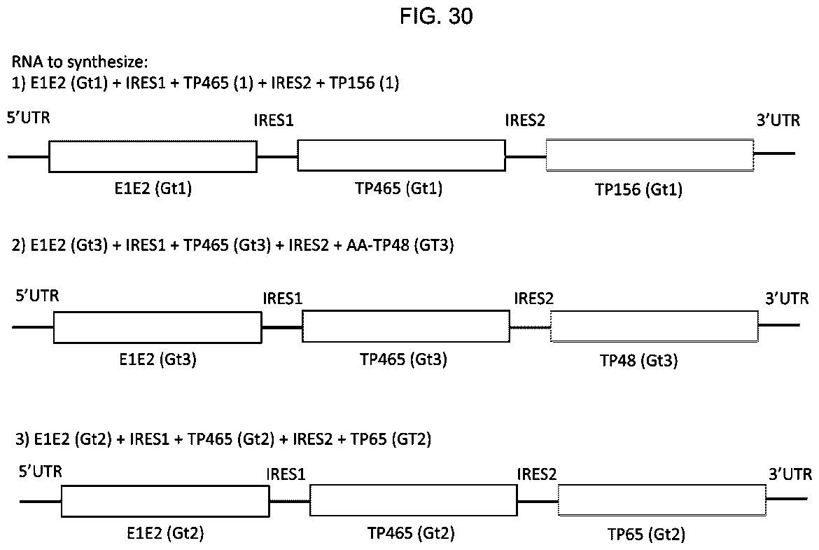

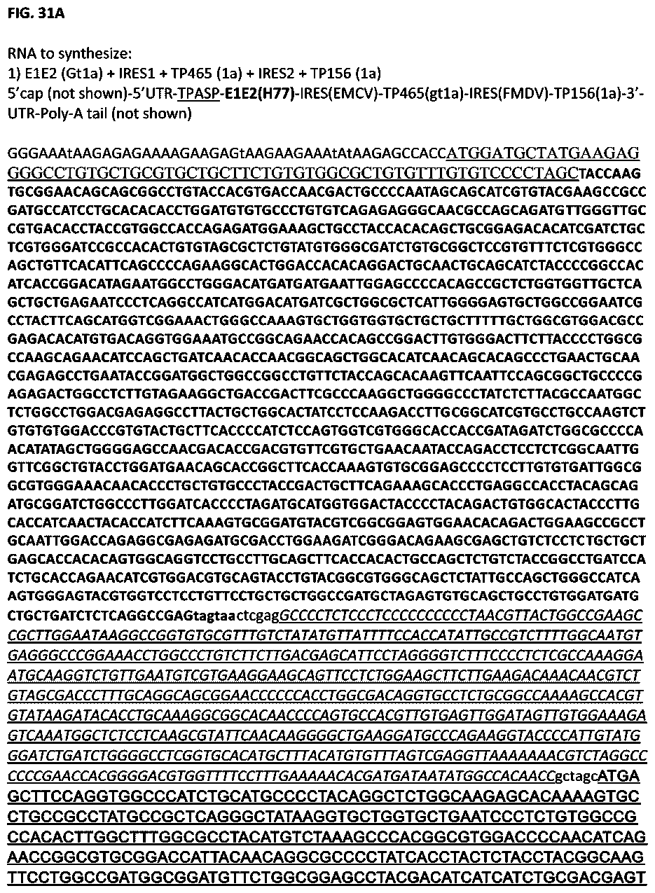

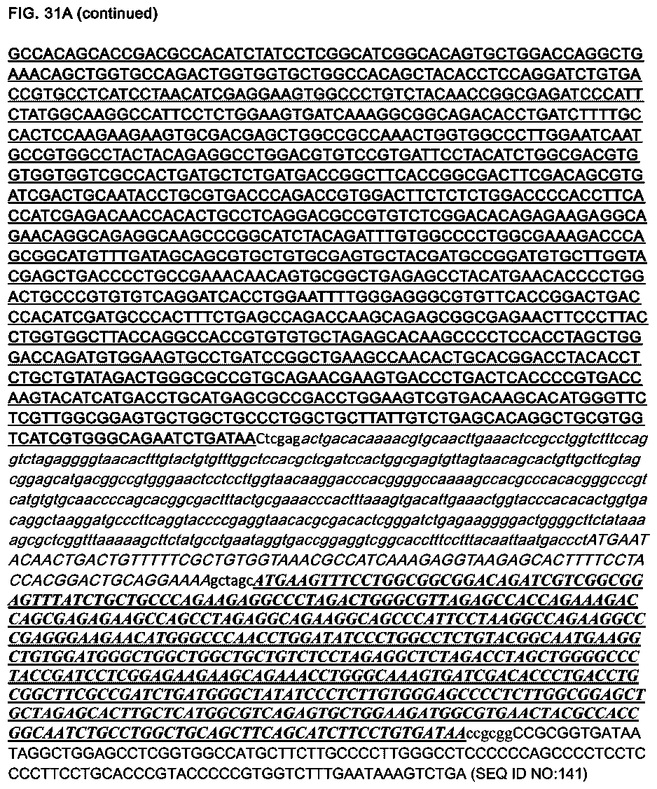

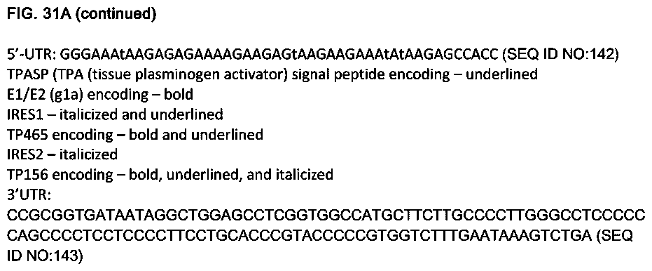

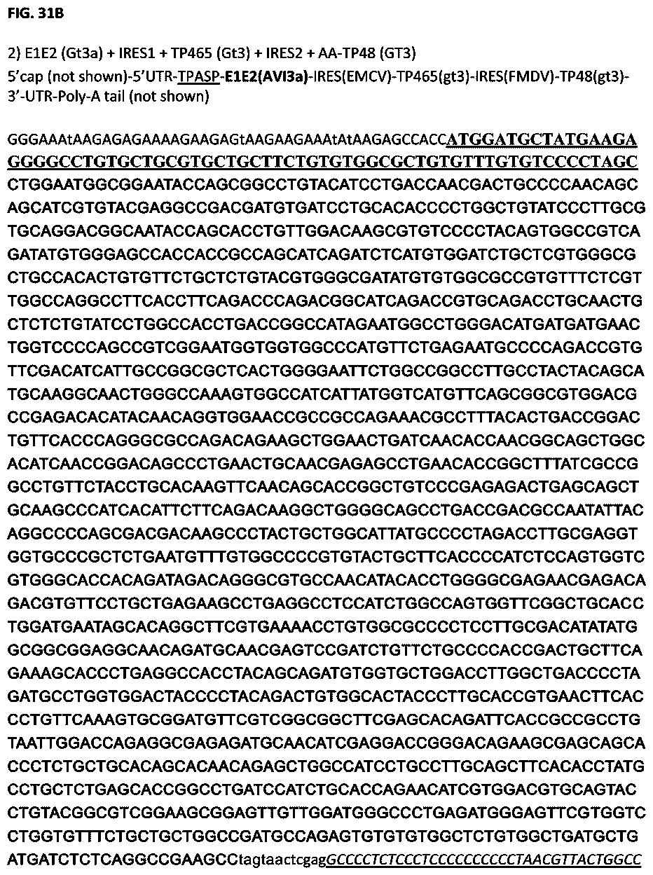

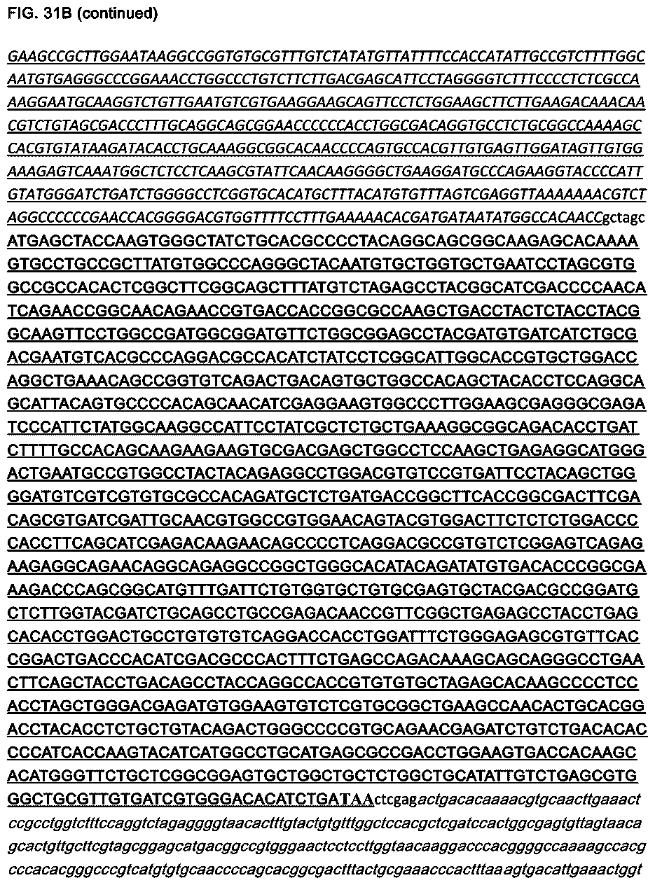

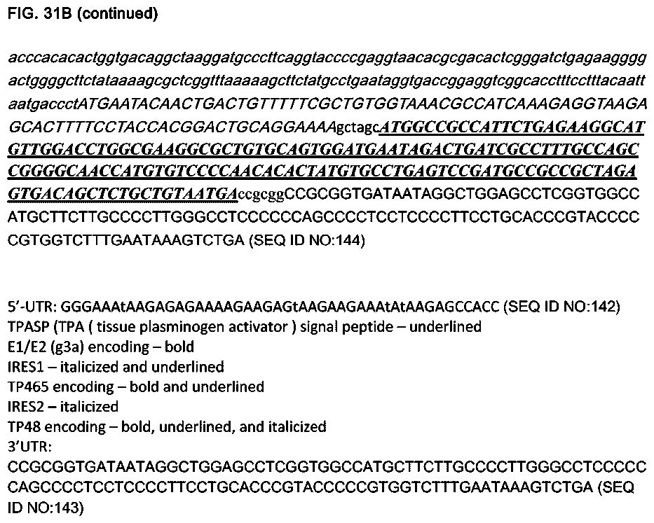

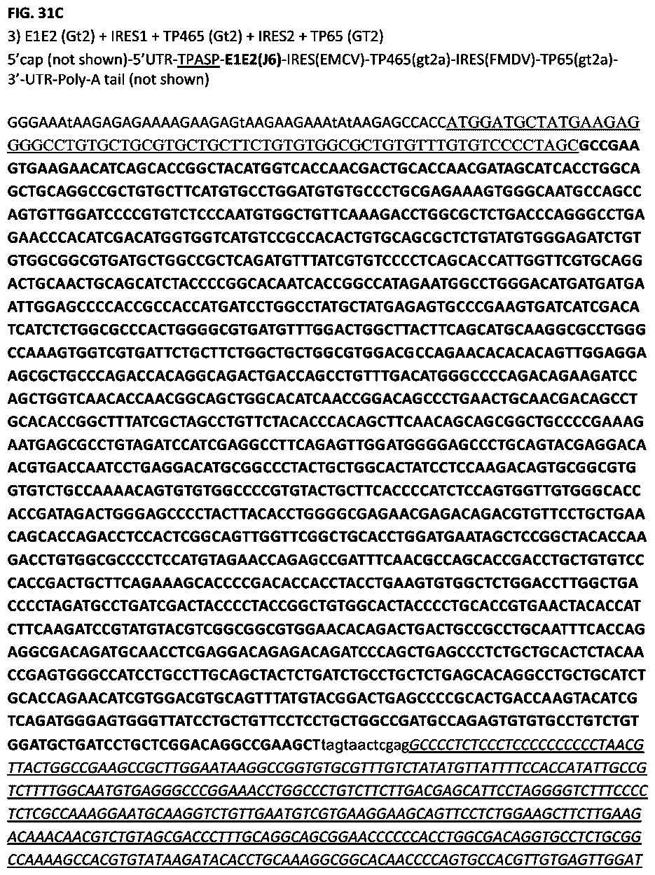

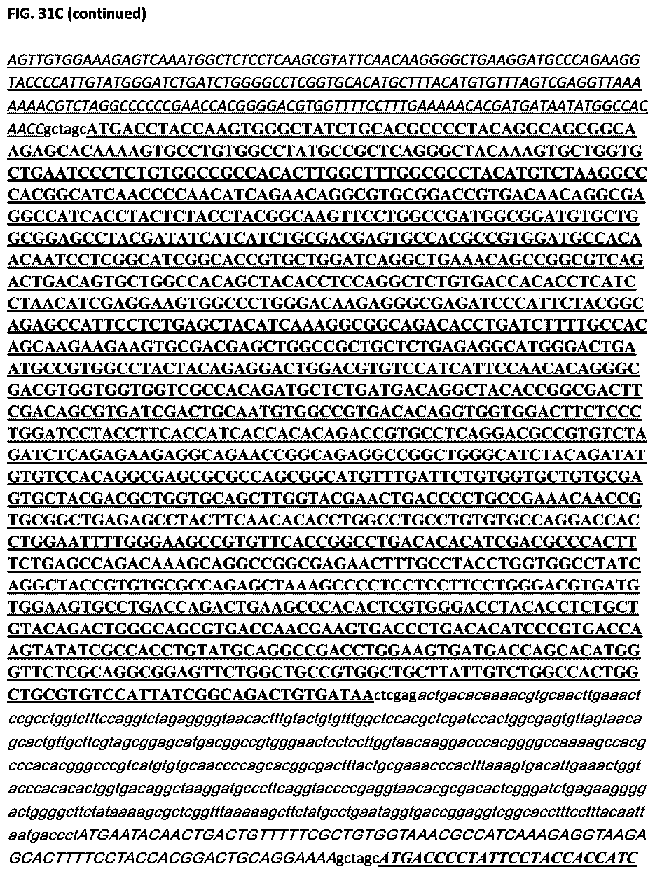

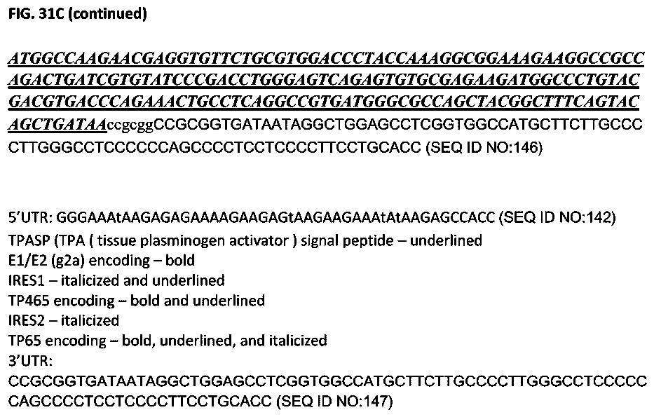

As another example, as depicted schematically in FIG. 30, in some cases, a composition of the present disclosure comprises: a) a first RNA molecule comprising: i) a nucleotide sequence encoding HCV E1 and E2 polypeptides of a first genotype (e.g., genotype 1); ii) a first IRES; iii) a nucleotide sequence encoding a TP465 polypeptide; iv) a second IRES; and v) a nucleotide sequence encoding a TP156 polypeptide; b) a second RNA molecule comprising: i) a nucleotide sequence encoding HCV E1 and E2 polypeptides of a second genotype (e.g., genotype 3); ii) a first IRES; iii) a nucleotide sequence encoding a TP465 polypeptide; iv) a second IRES; and v) a nucleotide sequence encoding a TP48 polypeptide; and c) a third RNA molecule comprising: i) a nucleotide sequence encoding HCV E1 and E2 polypeptides of a third genotype (e.g., genotype 2); ii) a first IRES; iii) a nucleotide sequence encoding a TP465 polypeptide; iv) a second IRES; and v) a nucleotide sequence encoding a TP65 polypeptide. The first and the second IRES of each RNA molecule can be the same or different. In some cases, the RNA molecules include a 5′ cap and a poly(A) tail. In some cases, the RNA molecules include a 5′UTR and a 3′UTR. In some cases, the RNA molecules do not include a 5′UTR or a 3′UTR. FIG. 31A-31C provide non-limiting examples of such nucleic acids (e.g., DNA encoding the RNA molecules or, where the Ts are replaced by Us, nucleotide sequences of the RNA molecules themselves).

For example, in some cases, a composition of the present disclosure comprises: a) a first RNA molecule comprising: i) a nucleotide sequence encoding HCV E1 and E2 polypeptides of genotype 1a (e.g., where the nucleotide sequence has at least 90%, at least 95%, at least 98%, at least 99%, or 100%, nucleotide sequence identity to the HCV E1/E2-encoding sequence depicted in FIG. 31A; ii) a first IRES, e.g., where the first IRES is an EMCV IRES (e.g., where the first IRES comprises a nucleotide sequence having at least 90%, at least 95%, at least 98%, at least 99%, or 100%, nucleotide sequence identity to the EMCV IRES depicted in FIG. 31A); iii) a nucleotide sequence encoding a TP465 polypeptide (e.g., where the nucleotide sequence has at least 90%, at least 95%, at least 98%, at least 99%, or 100%, nucleotide sequence identity to the TP465 polypeptide-encoding nucleotide sequence depicted in FIG. 31A); iv) a second IRES, e.g., where the second IRES is an FMDV IRES (e.g., where the second IRES comprises a nucleotide sequence having at least 90%, at least 95%, at least 98%, at least 99%, or 100%, nucleotide sequence identity to the FMDV IRES depicted in FIG. 31A); and iv) a nucleotide sequence encoding a TP156 polypeptide (e.g., where the nucleotide sequence has at least 90%, at least 95%, at least 98%, at least 99%, or 100%, nucleotide sequence identity to the TP156 polypeptide-encoding nucleotide sequence depicted in FIG. 31A); b) a second RNA molecule comprising: i) a nucleotide sequence encoding HCV E1 and E2 polypeptides of genotype 3a (e.g., where the nucleotide sequence has at least 90%, at least 95%, at least 98%, at least 99%, or 100%, nucleotide sequence identity to the HCV E1/E2-encoding sequence depicted in FIG. 31B); ii) a first IRES, e.g., where the first IRES is an EMCV IRES (e.g., where the first IRES comprises a nucleotide sequence having at least 90%, at least 95%, at least 98%, at least 99%, or 100%, nucleotide sequence identity to the EMCV IRES depicted in FIG. 31B); iii) a nucleotide sequence encoding a TP465 polypeptide (e.g., where the nucleotide sequence has at least 90%, at least 95%, at least 98%, at least 99%, or 100%, nucleotide sequence identity to the TP465 polypeptide-encoding nucleotide sequence depicted in FIG. 31B); iv) a second IRES, e.g., where the second IRES is an FMDV IRES (e.g., where the second IRES comprises a nucleotide sequence having at least 90%, at least 95%, at least 98%, at least 99%, or 100%, nucleotide sequence identity to the FMDV IRES depicted in FIG. 31B); and iv) a nucleotide sequence encoding a TP48 polypeptide (e.g., where the nucleotide sequence has at least 90%, at least 95%, at least 98%, at least 99%, or 100%, nucleotide sequence identity to the TP48 polypeptide-encoding nucleotide sequence depicted in FIG. 31B); and c) a third RNA molecule comprising: i) a nucleotide sequence encoding HCV E1 and E2 polypeptides of genotype 2a (e.g., where the nucleotide sequence has at least 90%, at least 95%, at least 98%, at least 99%, or 100%, nucleotide sequence identity to the HCV E1/E2-encoding sequence depicted in FIG. 31C); ii) a first IRES, e.g., where the first IRES is an EMCV IRES (e.g., where the first IRES comprises a nucleotide sequence having at least 90%, at least 95%, at least 98%, at least 99%, or 100%, nucleotide sequence identity to the EMCV IRES depicted in FIG. 31C); iii) a nucleotide sequence encoding a TP465 polypeptide (e.g., where the nucleotide sequence has at least 90%, at least 95%, at least 98%, at least 99%, or 100%, nucleotide sequence identity to the TP465 polypeptide-encoding nucleotide sequence depicted in FIG. 31C); iv) a second IRES, e.g., where the second IRES is an FMDV IRES (e.g., where the second IRES comprises a nucleotide sequence having at least 90%, at least 95%, at least 98%, at least 99%, or 100%, nucleotide sequence identity to the FMDV IRES depicted in FIG. 31C); and iv) a nucleotide sequence encoding a TP65 polypeptide (e.g., where the nucleotide sequence has at least 90%, at least 95%, at least 98%, at least 99%, or 100%, nucleotide sequence identity to the TP65 polypeptide-encoding nucleotide sequence depicted in FIG. 31C. In some cases, a tissue plasminogen activator signal peptide (TPASP)-encoding nucleotide sequence is included.

For example, in some cases, a composition of the present disclosure comprises: a) a first RNA molecule comprising: i) a nucleotide sequence encoding HCV E1 and E2 polypeptides of genotype 1a (e.g., where the nucleotide sequence encodes HCV E1 and E2 polypeptides having at least 90%, at least 95%, at least 98%, at least 99%, or 100%, amino acid sequence identity to the HCV E1 and E2 amino acid sequences depicted in FIG. 32A (where E2 is underlined)); ii) a first IRES, e.g., where the first IRES is an EMCV IRES (e.g., where the first IRES comprises a nucleotide sequence having at least 90%, at least 95%, at least 98%, at least 99%, or 100%, nucleotide sequence identity to the EMCV IRES depicted in FIG. 31A); iii) a nucleotide sequence encoding a TP465 polypeptide (e.g., where the nucleotide sequence encodes a TP465 polypeptide having at least 90%, at least 95%, at least 98%, at least 99%, or 100%, amino acid sequence identity to the TP465 polypeptide amino acid sequence depicted in FIG. 32B); iv) a second IRES, e.g., where the second IRES is an FMDV IRES (e.g., where the second IRES comprises a nucleotide sequence having at least 90%, at least 95%, at least 98%, at least 99%, or 100%, nucleotide sequence identity to the FMDV IRES depicted in FIG. 31A); and iv) a nucleotide sequence encoding a TP156 polypeptide (e.g., where the nucleotide sequence encodes a TP156 polypeptide having at least 90%, at least 95%, at least 98%, at least 99%, or 100%, amino sequence identity to the TP156 polypeptide amino acid sequence depicted in FIG. 32C); b) a second RNA molecule comprising: i) a nucleotide sequence encoding HCV E1 and E2 polypeptides of genotype 3a (e.g., where the nucleotide sequence encodes E1 and E2 polypeptides having at least 90%, at least 95%, at least 98%, at least 99%, or 100%, nucleotide sequence identity to the HCV E1 and E2 amino acid sequences depicted in FIG. 33A (where E2 is underlined)); ii) a first IRES, e.g., where the first IRES is an EMCV IRES (e.g., where the first IRES comprises a nucleotide sequence having at least 90%, at least 95%, at least 98%, at least 99%, or 100%, nucleotide sequence identity to the EMCV IRES depicted in FIG. 31B); iii) a nucleotide sequence encoding a TP465 polypeptide (e.g., where the nucleotide sequence encodes a TP465 polypeptide having at least 90%, at least 95%, at least 98%, at least 99%, or 100%, nucleotide sequence identity to the TP465 polypeptide amino acid sequence depicted in FIG. 33B); iv) a second IRES, e.g., where the second IRES is an FMDV IRES (e.g., where the second IRES comprises a nucleotide sequence having at least 90%, at least 95%, at least 98%, at least 99%, or 100%, nucleotide sequence identity to the FMDV IRES depicted in FIG. 31B); and iv) a nucleotide sequence encoding a TP48 polypeptide (e.g., where the nucleotide sequence encodes a TP48 polypeptide having at least 90%, at least 95%, at least 98%, at least 99%, or 100%, amino acid sequence identity to the TP48 polypeptide amino acid sequence depicted in FIG. 33C); and c) a third RNA molecule comprising: i) a nucleotide sequence encoding HCV E1 and E2 polypeptides of genotype 2a (e.g., where the nucleotide sequence encodes E1 and E2 polypeptides having at least 90%, at least 95%, at least 98%, at least 99%, or 100%, nucleotide sequence identity to the HCV E1 and E2 amino acid sequences depicted in FIG. 34A (where E2 is underlined)); ii) a first IRES, e.g., where the first IRES is an EMCV IRES (e.g., where the first IRES comprises a nucleotide sequence having at least 90%, at least 95%, at least 98%, at least 99%, or 100%, nucleotide sequence identity to the EMCV IRES depicted in FIG. 31C); iii) a nucleotide sequence encoding a TP465 polypeptide (e.g., where the nucleotide sequence encodes a TP465 polypeptide having at least 90%, at least 95%, at least 98%, at least 99%, or 100%, amino acid sequence identity to the TP465 polypeptide amino acid sequence depicted in FIG. 34B); iv) a second IRES, e.g., where the second IRES is an FMDV IRES (e.g., where the second IRES comprises a nucleotide sequence having at least 90%, at least 95%, at least 98%, at least 99%, or 100%, nucleotide sequence identity to the FMDV IRES depicted in FIG. 31C); and iv) a nucleotide sequence encoding a TP65 polypeptide (e.g., where the nucleotide sequence encodes a TP65 polypeptide having at least 90%, at least 95%, at least 98%, at least 99%, or 100%, amino acid sequence identity to the TP65 polypeptide amino acid sequence depicted in FIG. 34C. In some cases, TPASP-encoding nucleotide sequence is included.

### Compositions Comprising a Single Type of RNA Molecule

A composition comprising one or more RNA molecules of the present disclosure can comprise a single type of RNA molecule in which the encoded polypeptides (e.g., HCV E1 and/or E2; and optionally one or more additional polypeptides such as a heterologous T-cell epitope polypeptide(s) or a fusion polypeptide comprising 2 or more T-cell epitope polypeptides; and/or an HCV core polypeptide) are encoded in any of a variety of arrangements, and in which the encoded HCV E1 and/or E2 polypeptides are any of various HCV genotypes. A composition of the present disclosure can include a single type of RNA; i.e., all of the RNA molecules in the composition can encode the same polypeptides (e.g., HCV E1 and/or E2 polypeptides; optionally also one or more heterologous polypeptides). As used herein, a “single” RNA molecule refers to a single “type” or “species” of RNA molecule; i.e., all of the RNA molecules in the composition have the same nucleotide sequence. A composition comprising a single RNA molecule can comprise multiple copies of the RNA molecule. The following are non-limiting exemplary embodiments of a “single” RNA composition of the present disclosure.

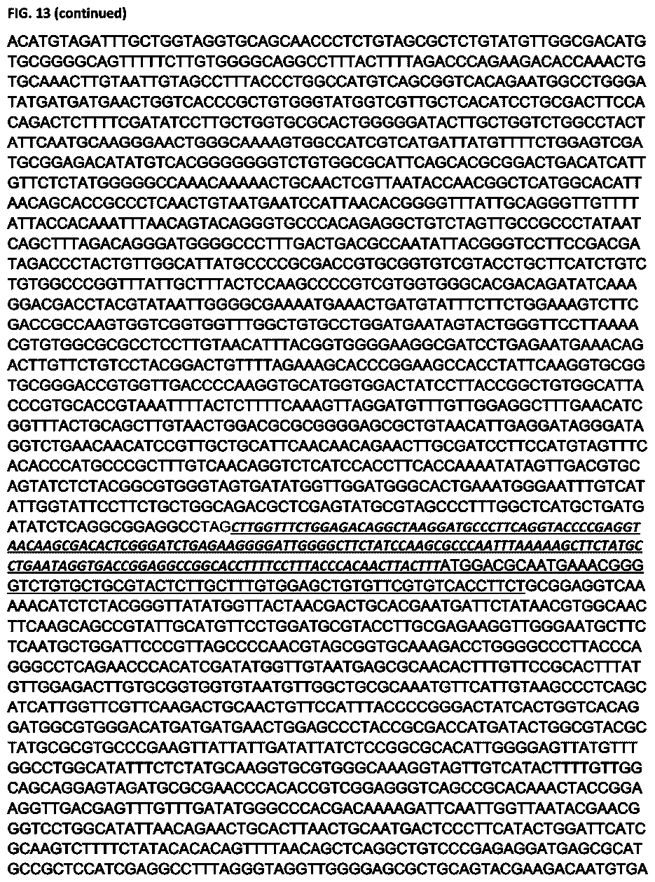

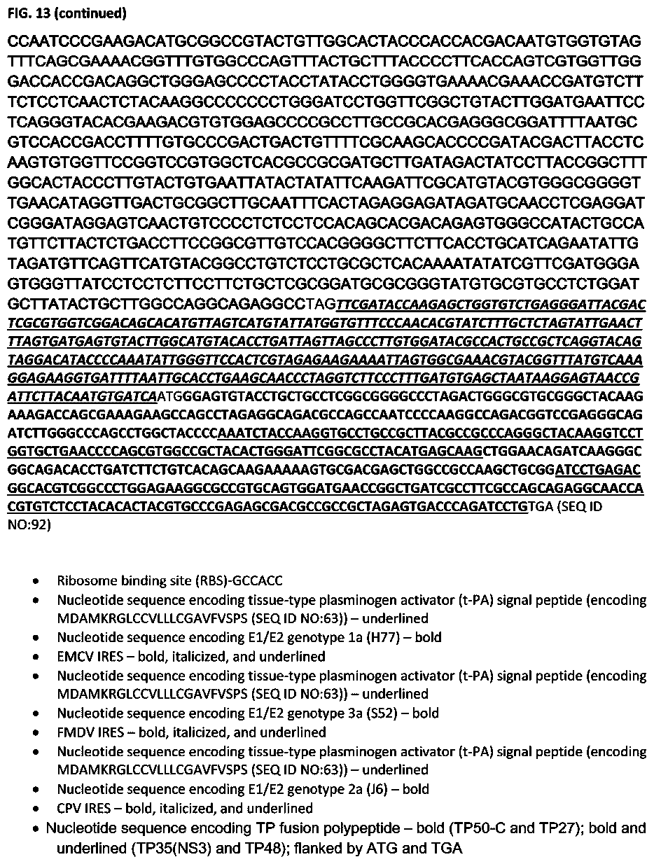

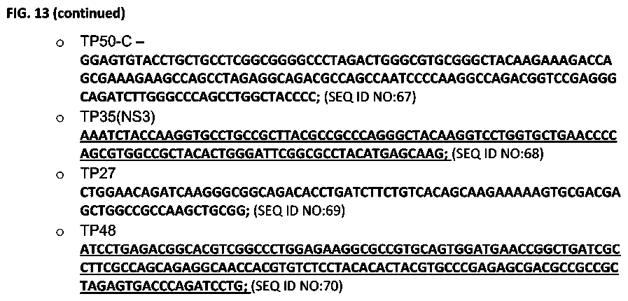

In some cases, an RNA molecule present in a composition of the present disclosure comprises, in order from 5′ to 3′: i) a nucleotide sequence encoding HCV E1 and/or E2 polypeptides of a first genotype; ii) a first IRES; iii) a nucleotide sequence encoding HCV E1 and E2 polypeptides of a second genotype; iv) a second IRES; v) a nucleotide sequence encoding HCV E1 and/or E2 polypeptides of a third genotype; vi) a third IRES; and vii) one or more nucleotide sequences encoding one or more heterologous polypeptides (e.g., T-cell epitope polypeptides). As an example, as illustrated schematically in FIG. 8E, an RNA molecule present in a composition of the present disclosure comprises, in order from 5′ to 3′: i) a nucleotide sequence encoding HCV E1 and E2 peptides of genotype 1; ii) a first IRES; iii) a nucleotide sequence encoding HCV E1 and E2 peptides of genotype 3; iv) a second IRES; v) a nucleotide sequence encoding HCV E1 and E2 peptides of genotype 2; vi) a third IRES; and vii) one or more nucleotide sequences encoding one or more heterologous polypeptides (e.g., one or more TPs) or a heterologous fusion polypeptide (e.g., a fusion polypeptide comprising two or more (e.g., 1, 2, 3, 4, 5, 6, 7, 8, 9, or 10 TPs). For example, (vii) can be a nucleotide sequence encoding a fusion polypeptide comprising, in order from N-terminus to C-terminus: a TP50-C polypeptide; a TP35-NS3 polypeptide; a TP27 polypeptide; and a TP48 polypeptide. The first, second, and third IRESs can be the same or different. As one non-limiting example, an RNA of the present disclosure can comprise the nucleotide sequence depicted in FIG. 13.

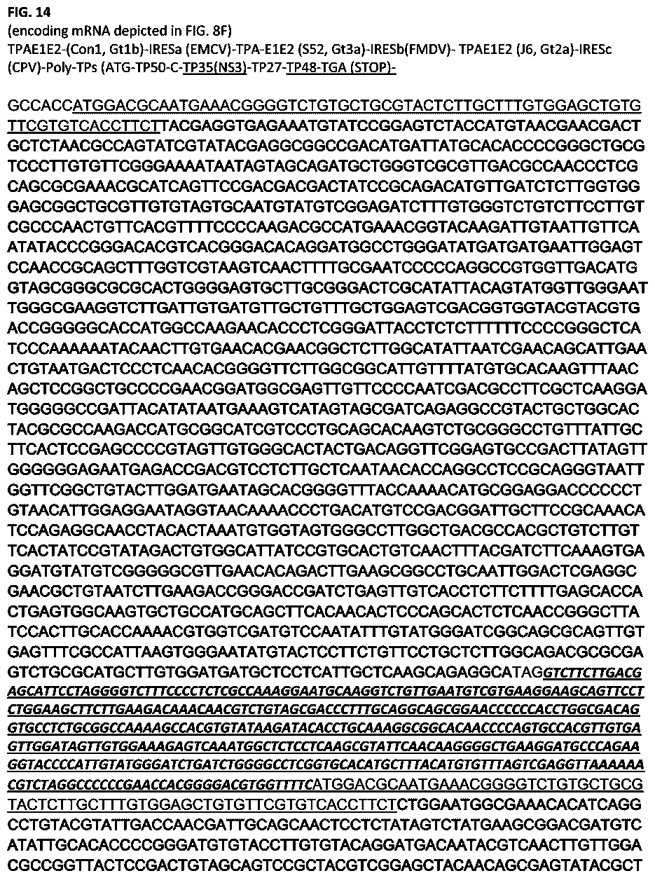

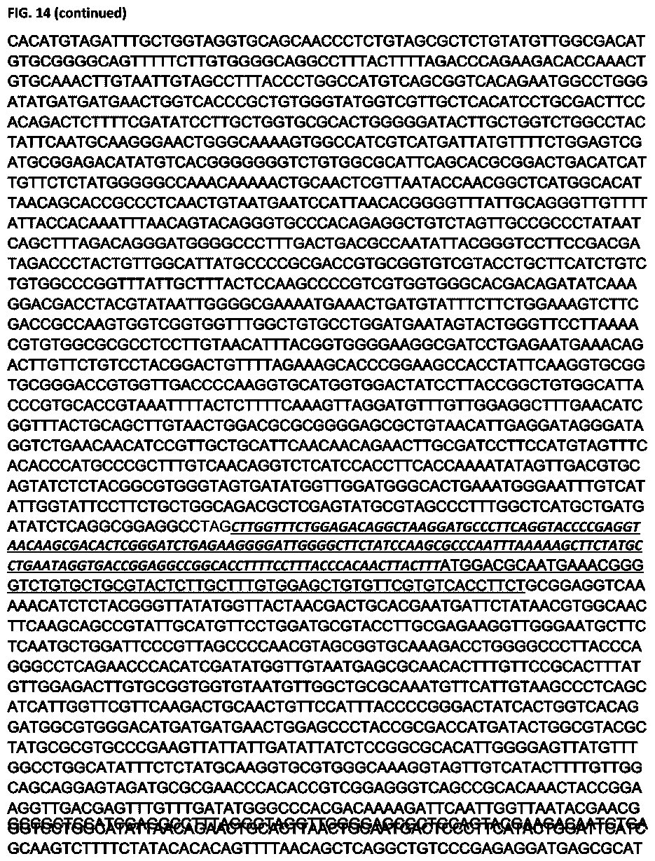

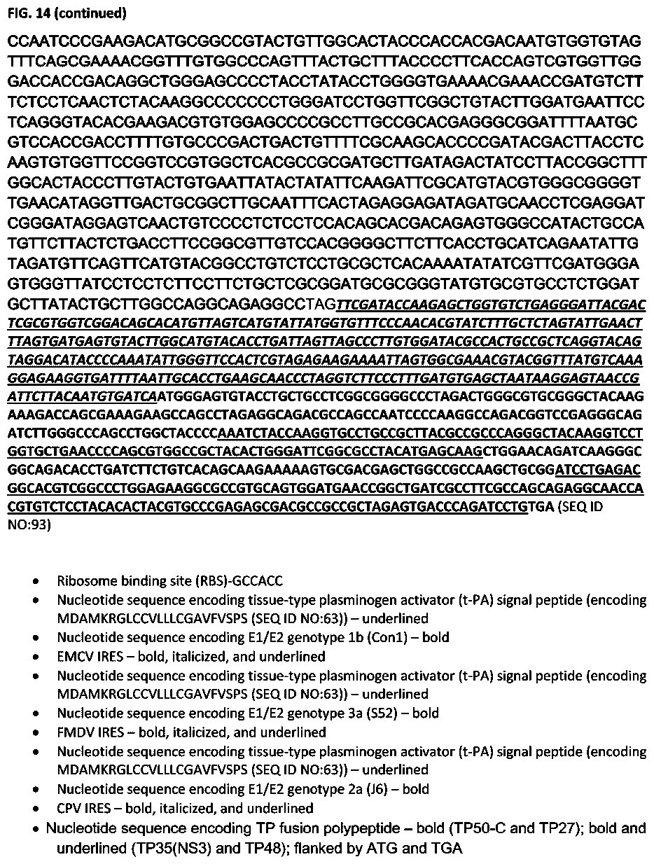

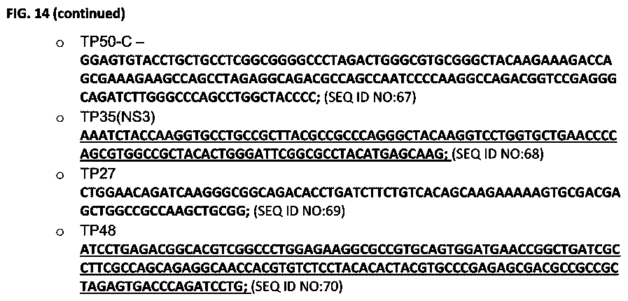

In some cases, an RNA molecule present in a composition of the present disclosure comprises, in order from 5′ to 3′: i) a nucleotide sequence encoding HCV E1 and/or E2 polypeptides of a first genotype; ii) a first IRES; iii) a nucleotide sequence encoding HCV E1 and E2 polypeptides of a second genotype; iv) a second IRES; v) a nucleotide sequence encoding HCV E1 and/or E2 polypeptides of a third genotype; vi) a third IRES; and vii) one or more nucleotide sequences encoding one or more heterologous polypeptides (e.g., T-cell epitope polypeptides). As an example, as illustrated schematically in FIG. 8F, n RNA molecule present in a composition of the present disclosure comprises, in order from 5′ to 3′: i) a nucleotide sequence encoding HCV E1 and E2 peptides of genotype 1b; ii) a first IRES; iii) a nucleotide sequence encoding HCV E1 and E2 peptides of genotype 3; iv) a second IRES; v) a nucleotide sequence encoding HCV E1 and E2 peptides of genotype 2; vi) a third IRES; and vii) one or more nucleotide sequences encoding one or more heterologous polypeptides (e.g., one or more TPs) or a heterologous fusion polypeptide (e.g., a fusion polypeptide comprising two or more (e.g., 1, 2, 3, 4, 5, 6, 7, 8, 9, or 10 TPs). For example, (vii) can be a nucleotide sequence encoding a fusion polypeptide comprising, in order from N-terminus to C-terminus: a TP50-C polypeptide; a TP35-NS3 polypeptide; a TP27 polypeptide; and a TP48 polypeptide. The first, second, and third IRESs can be the same or different. As one non-limiting example, an RNA of the present disclosure can comprise the nucleotide sequence depicted in FIG. 14.

In some cases, an RNA molecule present in a composition of the present disclosure comprises, in order from 5′ to 3′: i) a nucleotide sequence encoding HCV E1 and E2 polypeptides of a first genotype; ii) a first IRES; iii) a nucleotide sequence encoding HCV E1 and E2 polypeptides of a second genotype; iv) a second IRES; v) a nucleotide sequence encoding HCV E1 and E2 polypeptides of a third genotype; and b) the second polypeptide-encoding region vi) a third IRES; and vii) one or more nucleotide sequences encoding a TP50-C polypeptide, a TP35-NS3 polypeptide, a TP42 polypeptide, and a TP48 polypeptide. In some cases, each of the nucleotide sequences encoding the TP50-C polypeptide, the TP35-NS3 polypeptide, the TP42 polypeptide, and the TP48 polypeptide are separated by an IRES.

In some cases, an RNA molecule present in a composition of the present disclosure comprises, in order from 5′ to 3′: i) a nucleotide sequence encoding HCV E1 and/or E2 polypeptides of a first genotype; ii) a first IRES; iii) a nucleotide sequence encoding HCV E1 and/or E2 polypeptides of a second genotype; iv) a second IRES; v) a nucleotide sequence encoding HCV E1 and/or E2 polypeptides of a third genotype; and b) the second polypeptide-encoding region vi) a third IRES; and vii) one or more nucleotide sequences encoding a TP50-C polypeptide, a TP35-NS3 polypeptide, a TP27 polypeptide, and a TP48 polypeptide. In some cases, each of the nucleotide sequences encoding the TP50-C polypeptide, the TP35-NS3 polypeptide, the TP27 polypeptide, and the TP48 polypeptide are separated by an IRES.

In some cases, an RNA molecule present in a composition of the present disclosure comprises, in order from 5′ to 3′: i) a nucleotide sequence encoding HCV E1 and E2 polypeptides of a first genotype; ii) a first IRES; iii) a nucleotide sequence encoding HCV E1 and/or E2 polypeptides of a second genotype; iv) a second IRES; v) a nucleotide sequence encoding HCV E1 and/or E2 polypeptides of a third genotype; and b) the second polypeptide-encoding region vi) a third IRES; and vii) one or more nucleotide sequences encoding a fusion polypeptide encoding 2 or more T-cell epitope polypeptides.

In some cases, an RNA molecule present in a composition of the present disclosure comprises, in order from 5′ to 3′: i) a nucleotide sequence encoding HCV E1 and E2 polypeptides of genotype 1; ii) a first IRES; iii) a nucleotide sequence encoding HCV E1 and E2 polypeptides of genotype 3; iv) a second IRES; v) a nucleotide sequence encoding HCV E1 and E2 polypeptides of genotype 2; and b) the second polypeptide-encoding region vi) a third IRES; and vii) one or more nucleotide sequences encoding one or more heterologous polypeptides or the fusion polypeptide.

In some cases, a composition of the present disclosure comprises a single type of RNA molecule, where the single type of RNA molecule comprises: i) a nucleotide sequence encoding one or more HCV polypeptides (e.g., an HCV E1 polypeptide; an HCV E2 polypeptide; a soluble HCV E1 (sE1) polypeptide; a soluble HCV E2 (sE2) polypeptide; an HCV E1/E2 heterodimer; a soluble E1/E2 heterodimer (sE1/E2); or a Core-E1-E2 polyprotein), and optionally including a heterologous signal peptide (e.g., t-PA); ii) an IRES (e.g., EMCV IRES); and iii) a nucleotide sequence encoding a TP fusion polypeptide comprising TP50-C; TP35-NS3; TP27; and TP48. The mRNA molecule can include a Cap sequence and can include a poly(A) tail. For example, as depicted schematically in FIG. 22E, a composition of the present disclosure comprises an RNA molecule comprising: i) a nucleotide sequence encoding one or more HCV polypeptides (e.g., an HCV E1 polypeptide; an HCV E2 polypeptide; a soluble HCV E1 (sE1) polypeptide; a soluble HCV E2 (sE2) polypeptide; an HCV E1/E2 heterodimer; a soluble E1/E2 heterodimer (sE1/E2); or a Core-E1-E2 polyprotein) of genotype 1, 2, 3, 4, 5, or 6, and optionally including a heterologous signal peptide (e.g., t-PA); ii) an IRES (e.g., EMCV IRES); and iii) a nucleotide sequence encoding a TP fusion polypeptide comprising TP50-C; TP35-NS3; TP27; and TP48. In some cases, a composition of the present disclosure comprises an RNA molecule comprising: i) a nucleotide sequence encoding one or more HCV polypeptides (e.g., an HCV E1 polypeptide; an HCV E2 polypeptide; a soluble HCV E1 (sE1) polypeptide; a soluble HCV E2 (sE2) polypeptide; an HCV E1/E2 heterodimer; a soluble E1/E2 heterodimer (sE1/E2); or a Core-E1-E2 polyprotein) of genotype 1, and optionally including a heterologous signal peptide (e.g., t-PA); ii) an IRES (e.g., EMCV IRES); and iii) a nucleotide sequence encoding a TP fusion polypeptide comprising TP50-C; TP35-NS3; TP27; and TP48. The mRNA molecule can include a Cap sequence and can include a poly(A) tail. In other cases, a composition of the present disclosure comprises an RNA molecule comprising: i) a nucleotide sequence encoding one or more HCV polypeptides (e.g., an HCV E1 polypeptide; an HCV E2 polypeptide; a soluble HCV E1 (sE1) polypeptide; a soluble HCV E2 (sE2) polypeptide; an HCV E1/E2 heterodimer; a soluble E1/E2 heterodimer (sE1/E2); or a Core-E1-E2 polyprotein) of genotype 3, and optionally including a heterologous signal peptide (e.g., t-PA); ii) an IRES (e.g., EMCV IRES); and iii) a nucleotide sequence encoding a TP fusion polypeptide comprising TP50-C; TP35-NS3; TP27; and TP48. The mRNA molecule can include a Cap sequence and can include a poly(A) tail. In some cases, the genotype 1 E1 and/or the E2 polypeptides are H77 polypeptides, as described above. In some cases, the genotype 1 E1 and/or the E2 polypeptides are soluble H77 polypeptides, as described above. In some cases, the genotype 3 E1 and/or the E2 polypeptides are S52 polypeptides, as described above. In some cases, the genotype 3 E1 and/or the E2 polypeptides are soluble S52 polypeptides, as described above. In other cases, a composition of the present disclosure comprises an RNA molecule comprising: i) a nucleotide sequence encoding one or more HCV polypeptides (e.g., an HCV E1 polypeptide; an HCV E2 polypeptide; a soluble HCV E1 (sE1) polypeptide; a soluble HCV E2 (sE2) polypeptide; an HCV E1/E2 heterodimer; a soluble E1/E2 heterodimer (sE1/E2); or a Core-E1-E2 polyprotein) of genotype 2, and optionally including a heterologous signal peptide (e.g., t-PA); ii) an IRES (e.g., EMCV IRES); and iii) a nucleotide sequence encoding a TP fusion polypeptide comprising TP50-C; TP35-NS3; TP27; and TP48. In other cases, a composition of the present disclosure comprises an RNA molecule comprising: i) a nucleotide sequence encoding one or more HCV polypeptides (e.g., an HCV E1 polypeptide; an HCV E2 polypeptide; a soluble HCV E1 (sE1) polypeptide; a soluble HCV E2 (sE2) polypeptide; an HCV E1/E2 heterodimer; a soluble E1/E2 heterodimer (sE1/E2); or a Core-E1-E2 polyprotein) of genotype 4, and optionally including a heterologous signal peptide (e.g., t-PA); ii) an IRES (e.g., EMCV IRES); and iii) a nucleotide sequence encoding a TP fusion polypeptide comprising TP50-C; TP35-NS3; TP27; and TP48. In other cases, a composition of the present disclosure comprises an RNA molecule comprising: i) a nucleotide sequence encoding one or more HCV polypeptides (e.g., an HCV E1 polypeptide; an HCV E2 polypeptide; a soluble HCV E1 (sE1) polypeptide; a soluble HCV E2 (sE2) polypeptide; an HCV E1/E2 heterodimer; a soluble E1/E2 heterodimer (sE1/E2); or a Core-E1-E2 polyprotein) of genotype 5, and optionally including a heterologous signal peptide (e.g., t-PA); ii) an IRES (e.g., EMCV IRES); and iii) a nucleotide sequence encoding a TP fusion polypeptide comprising TP50-C; TP35-NS3; TP27; and TP48. In other cases, a composition of the present disclosure comprises an RNA molecule comprising: i) a nucleotide sequence encoding one or more HCV polypeptides (e.g., an HCV E1 polypeptide; an HCV E2 polypeptide; a soluble HCV E1 (sE1) polypeptide; a soluble HCV E2 (sE2) polypeptide; an HCV E1/E2 heterodimer; a soluble E1/E2 heterodimer (sE1/E2); or a Core-E1-E2 polyprotein) of genotype 6, and optionally including a heterologous signal peptide (e.g., t-PA); ii) an IRES (e.g., EMCV IRES); and iii) a nucleotide sequence encoding a TP fusion polypeptide comprising TP50-C; TP35-NS3; TP27; and TP48. The mRNA molecule can include a Cap sequence and can include a poly(A) tail.

In some cases, a composition of the present disclosure comprises a single type of RNA molecule, where the single type of RNA molecule comprises a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein); and ii) a TP fusion polypeptide comprising TP50-C; TP35-NS3; TP27; and TP48. The mRNA molecule can include a Cap sequence and can include a poly(A) tail. As an example, as depicted schematically in FIG. 22F, in some cases, a composition of the present disclosure an RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 1, 2, 3, 4, 5, or 6; and ii) a TP fusion polypeptide comprising TP50-C; TP35-NS3; TP27; and TP48. In some cases, a composition of the present disclosure an RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 1; and ii) a TP fusion polypeptide comprising TP50-C; TP35-NS3; TP27; and TP48. The mRNA molecule can include a Cap sequence and can include a poly(A) tail. As another example, in some cases, a composition of the present disclosure an RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 3; and ii) a TP fusion polypeptide comprising TP50-C; TP35-NS3; TP27; and TP48. The mRNA molecule can include a Cap sequence and can include a poly(A) tail. In some cases, the genotype 1 E1 and/or the E2 polypeptides are H77 polypeptides, as described above. In some cases, the genotype 1 E1 and/or the E2 polypeptides are soluble H77 polypeptides, as described above. In some cases, the genotype 3 E1 and/or the E2 polypeptides are S52 polypeptides, as described above. In some cases, the genotype 3 E1 and/or the E2 polypeptides are soluble S52 polypeptides, as described above. In some cases, a composition of the present disclosure an RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 2; and ii) a TP fusion polypeptide comprising TP50-C; TP35-NS3; TP27; and TP48. The mRNA molecule can include a Cap sequence and can include a poly(A) tail. In some cases, a composition of the present disclosure an RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 4; and ii) a TP fusion polypeptide comprising TP50-C; TP35-NS3; TP27; and TP48. The mRNA molecule can include a Cap sequence and can include a poly(A) tail. In some cases, a composition of the present disclosure an RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 5; and ii) a TP fusion polypeptide comprising TP50-C; TP35-NS3; TP27; and TP48. The mRNA molecule can include a Cap sequence and can include a poly(A) tail. In some cases, a composition of the present disclosure an RNA molecule comprising a nucleotide sequence encoding a fusion polypeptide comprising: i) one or more HCV polypeptides (e.g., an HCV E1 polypeptide; an HCV E2 polypeptide; an sE1 polypeptide; an sE2 polypeptide; an HCV E1/E2 heterodimer; an sE1/E2 heterodimer; or a Core-E1-E2 polyprotein) of genotype 6; and ii) a TP fusion polypeptide comprising TP50-C; TP35-NS3; TP27; and TP48. The mRNA molecule can include a Cap sequence and can include a poly(A) tail.

Generating mRNA Molecules

Polynucleotides, primary constructs, and an RNA molecule according to the present disclosure can be prepared according to any available technique including, but not limited to chemical synthesis, enzymatic synthesis (e.g., in vitro transcription (IVT)), enzymatic or chemical cleavage of a longer precursor. Methods of synthesizing RNAs are known in the art (see, e.g., Gait, M. J. (ed.) Oligonucleotide Synthesis: A Practical Approach, Oxford [Oxfordshire], Washington, D.C.: IRL Press, 1984; and Herdewijn, P. (ed.) Oligonucleotide Synthesis: Methods and Applications, Methods in Molecular Biology, v. 288 (Clifton, N.J.) Totowa, N.J.: Humana Press, 2005. The process of design and synthesis of the primary constructs generally includes the steps of gene construction, mRNA production (either with or without modifications) and purification. In the enzymatic synthesis method, a target polynucleotide sequence encoding the polypeptides (e.g., HCV E1 and/or E2; heterologous polypeptides) is first selected for incorporation into a vector which will be amplified to produce a cDNA template. Optionally, the target polynucleotide sequence and/or any flanking sequences may be codon optimized. The cDNA template is then used to produce mRNA through in vitro transcription (IVT). After production, the mRNA may undergo purification.

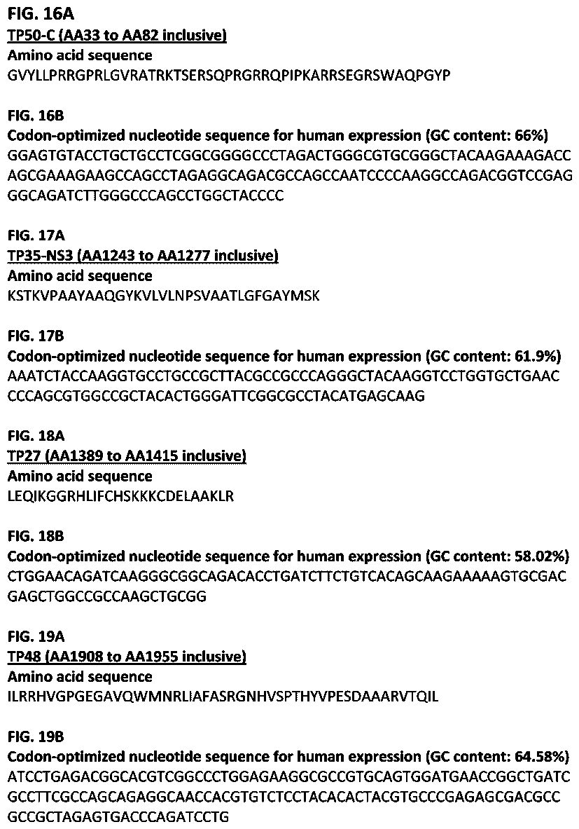

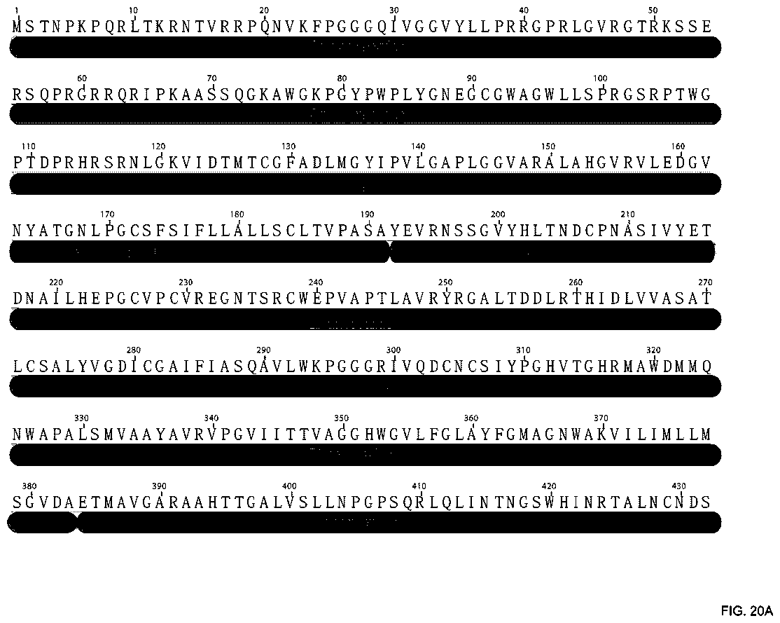

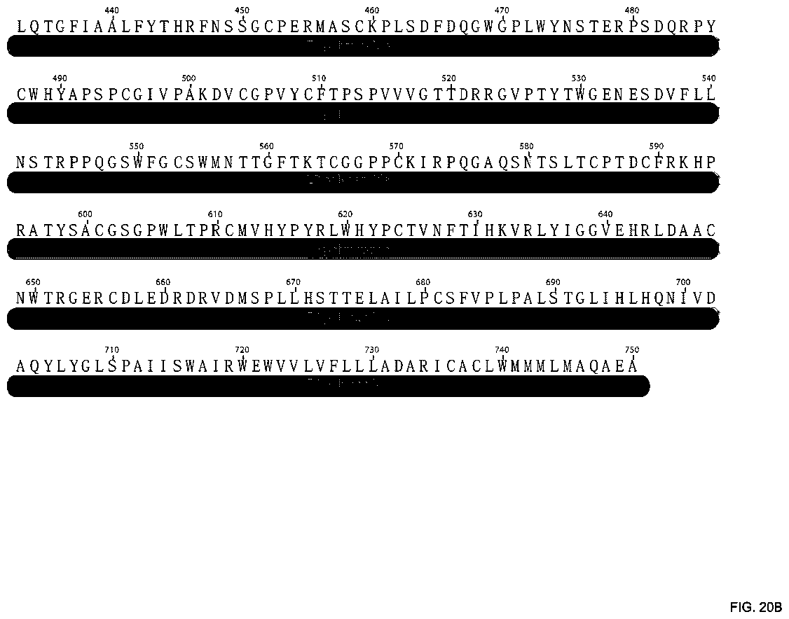

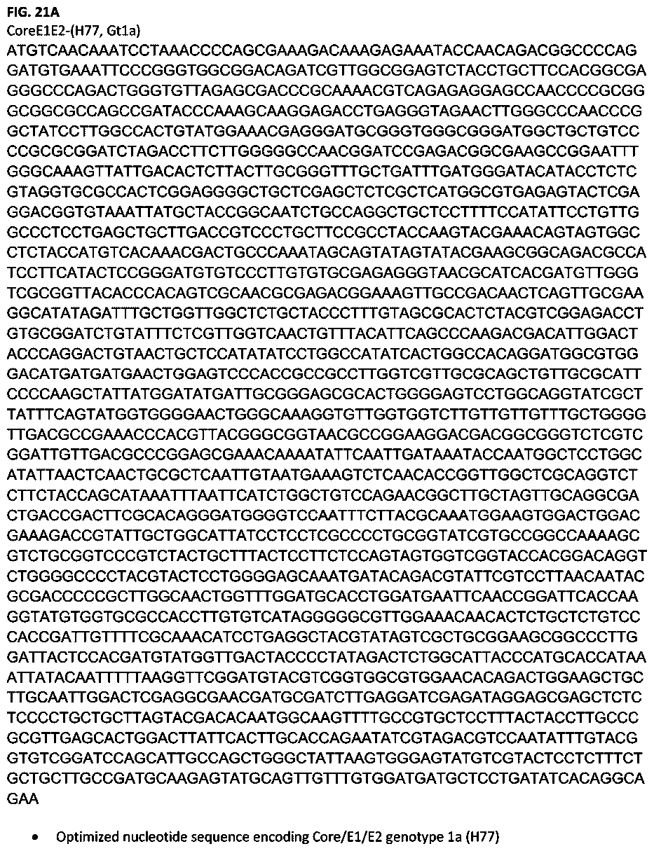

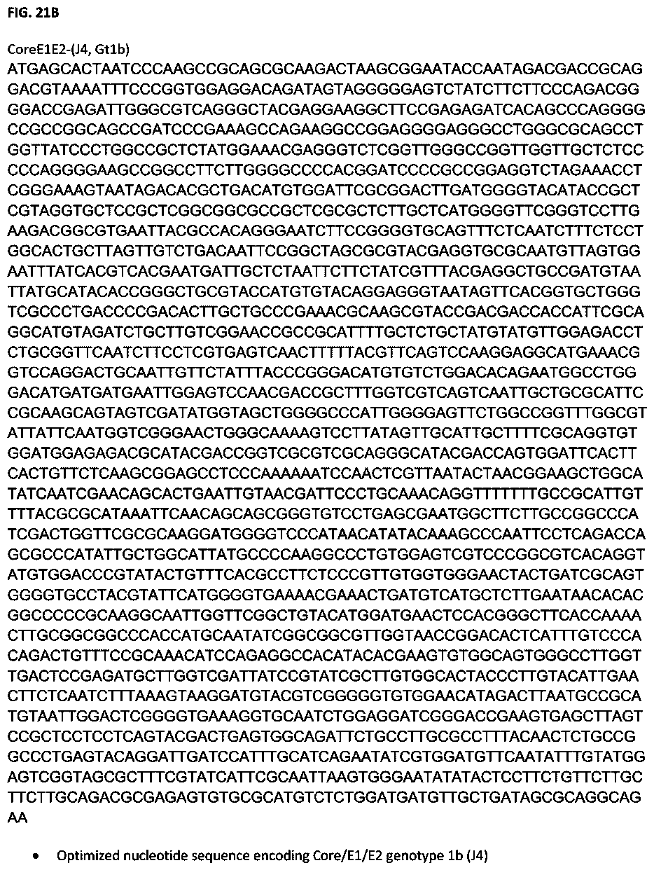

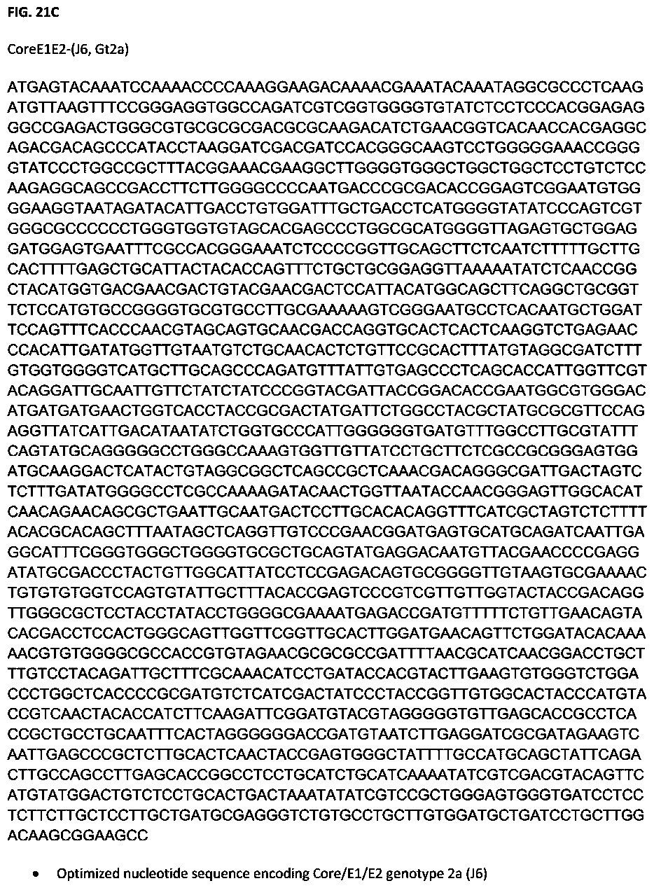

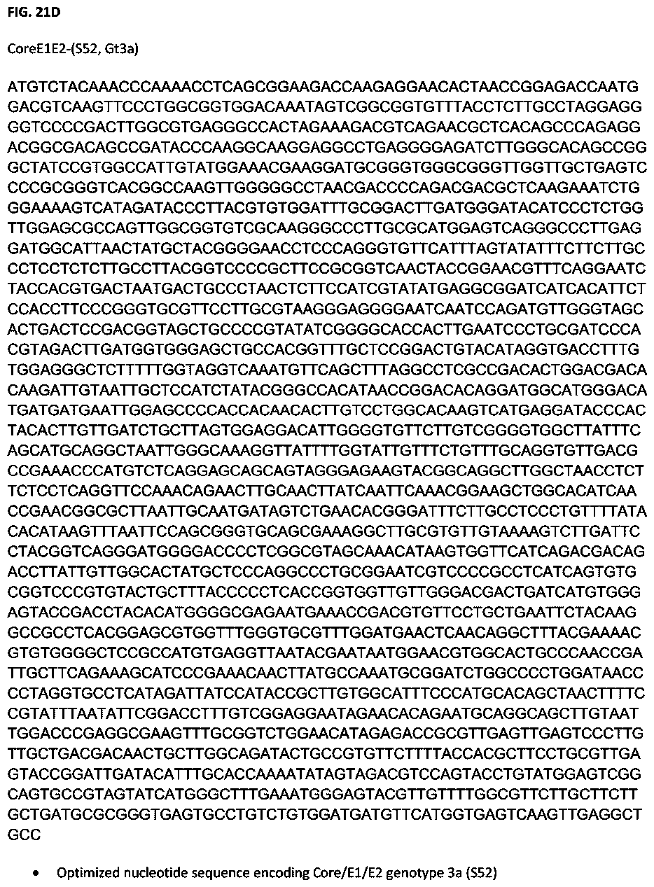

An RNA molecule can be designed such that it is codon optimized for expression in mammalian cells, e.g., in human cells. Codon usage tables are readily available, for example, at the “Codon Usage Database” available at www [dot]kazusa[dot] or [dot]jp[forwardslash]codon. FIG. 16B, FIG. 17B, FIG. 18B, and FIG. 19B provide examples of nucleotide sequences encoding various TPs, where the nucleotide sequences are codon optimized for expression in human cells. Examples of nucleotide sequences, codon optimized for expression in human cells, encoding HCV core-E1-E2 are provided in FIG. 21A-21D.

As is well known in the art, a polypeptide coding region can include a start codon (ATG), and may also include one or more stop codons (e.g., TGA, TAA, or TAG).

A complementary DNA (cDNA) template may be synthesized by having a linearized plasmid undergo polymerase chain reaction (PCR). RNA molecules can be generated using any method, a number of which are well known to those skilled in the art. For example, RNA production may include one or more of: in vitro (cell-free) transcription, cDNA template removal, purification, and mRNA capping and/or tailing reactions. See, e.g., U.S. Pat. No. 9,827,332.

For in vitro transcription, a cDNA template encoding an RNA can be transcribed in an in vitro cell-free transcription system. The in vitro cell-free system typically comprises a transcription buffer, nucleotide triphosphates (NTPs), an RNase inhibitor, and an RNA polymerase. The NTPs may be selected from, but are not limited to, natural and unnatural (modified) NTPs. The RNA polymerase may be selected from, but is not limited to, T7 RNA polymerase, T3 RNA polymerase and mutant polymerases such as RNA polymerases able to incorporate modified NTPs. Once the RNA molecules are generated, the cDNA may be removed, e.g., by treatment with a deoxyribonuclease I. The RNA produced may be subjected to a method such as a high-performance liquid chromatography (HPLC)-based method such as strong anion exchange HPLC, weak anion exchange HPLC, reverse phase HPLC, and hydrophobic interaction HPLC.

The RNAs produced will generally be at least 85% pure, at least 90% pure, at least 95% pure, at least 98% pure, or at least 99% pure.

formulations

As noted above, a composition of the present disclosure comprises: a) one or more RNA molecules, as described above; and b) one or more of: i) a lipid; ii) a polymer; iii) a peptide; iv) a viral-like particle (VLP); and v) a cationic nanoemulsion. A composition of the present disclosure can be in the form of a liposome, a lipid nanoparticle, and the like. In some cases, a composition of the present disclosure comprises: a) one or more RNA molecules, as described above; and ii) a poly(lactide-co-glycolide). In some cases, a composition of the present disclosure comprises: a) one or more RNA molecules, as described above; and ii) a polyalkyleneimine. See, e.g., Zeng et al. (2020) Formulation and Delivery Technologies for mRNA Vaccines. In: Current Topics in Microbiology and Immunology. Springer, Berlin, Heidelberg.

Other agents that can be formulated with one or more RNA molecules include viral-like particles (VLP), peptides, and cationic nanoemulsions. Peptides include, e.g., cationic peptides that are rich in Lys and/or Arg; and anionic peptides conjugated to positively-charged polymers, and the like. Suitable peptides include, e.g., protamine, cationic cell-penetrating peptides. Cationic nanoemulsions include, e.g., MF59.

A formulation of the present disclosure can include one or more excipients, each in an amount that together increases the stability of a nucleic acid of the present disclosure, increases cell transfection by a nucleic acid of the present disclosure, increases the expression of a protein(s) encoded by a nucleic acid of the present disclosure, and/or alters the release profile of a protein(s) encoded by a nucleic acid of the present disclosure. In some cases, a nucleic acid of the present disclosure is formulated using self-assembled nucleic acid nanoparticles.

A formulation of the present disclosure can include one or more different nucleic acids of the present disclosure. For example, a formulation of the present disclosure can include 1, 2, 3, 4, 5, or more than 5, different nucleic acids of the present disclosure, where the different nucleic acids differ from one another in nucleotide sequence.

### Polymers

In some cases, a composition of the present disclosure comprises one or more polymers. Suitable polymers include polyamines, dendrimers, and copolymers. Suitable polymers include, e.g., polyethylene glycol, polyglycolide, polyvinyl alcohol, polyvinyl pyrrolidone, polylactide, poly(lactide-co-glycolide), polycaprolactone, polysorbate, polyethylene oxide, polypropylene oxide, poly(ethylene oxide-co-propylene oxide), poloxamer, poloxamine, poly(oxyethylated) glycerol, poly(oxyethylated) sorbitol, poly(oxyethylated) glucose, and polyethyleneimine. Suitable polymers include polysaccharides. In some cases, the polymer is polyethyleneimine (PEI). In some cases, the polymer is polyamidoamine (PAMAM) dendrimer. In some cases, the polymer is poly(lactide-co-glycolide) (PLGA). In some cases, the polymer is the block copolymer poly(ethylene glycol)-block-poly(lactic-co-glycolic acid) (PEG-b-PLGA).

In some cases, a composition of the present disclosure comprises both a lipid and a polymer. For example, a composition of the present disclosure can include N-bis(2-hydroxyethyl)-N-methyl-N-(2-cholesteryloxy-carbonyl aminoethyl) ammonium bromide (BHEM-Chol), PEG-b-PLGA, and PLGA.

### Lipidoids

The synthesis of lipidoids has been extensively described and formulations containing these compounds are particularly suited for delivery of a nucleic acid of the present disclosure. Complexes, micelles, liposomes or particles can be prepared containing these lipidoids and therefore, can result in an effective delivery of a nucleic acid of the present disclosure, as judged by the production of an encoded protein, following the injection of a lipidoid formulation via localized and/or systemic routes of administration. Lipidoid complexes of a nucleic acid of the present disclosure can be administered by various means including, but not limited to, intravenous, intramuscular, or subcutaneous routes. Suitable lipidoids include, e.g., 98N12-5, C12-200, and MD1.

The lipidoid referred to herein as “98N12-5” is disclosed in Akinc et al., Mol Ther. 2009 17:872-879). The structure of 98N12-5 is as follows:

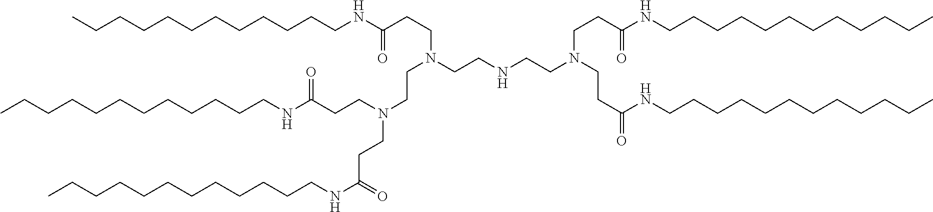 [SMILES: CCCCCCCCCCCCNC(=O)CCN(CCNCCN(CCC(=O)NCCCCCCCCCCCC)CCN(CCC(=O)NCCCCCCCCCCCC)CCC(=O)NCCCCCCCCCCCC)CCC(=O)NCCCCCCCCCCCC]

The lipidoid referred to herein as “C12-200” is disclosed by Love et al., Proc Natl Acad Sci USA. 2010 107:1864-1869; and Liu and Huang, Molecular Therapy. 2010 669-670. The structure of C12-200 is as follows:

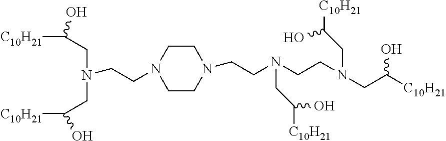

The lipidoid referred to herein as “MD1” is a diketopiperazine-based ionizable lipid, and is also known as cKK-E12. The compound cKK-E12 has the following structure:

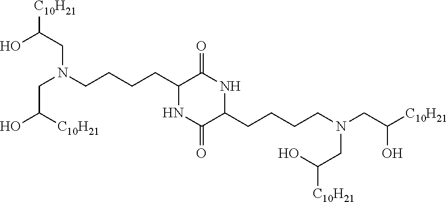

A lipidoid formulation can include particles comprising either 3 or 4 or more components in addition to a nucleic acid of the present disclosure. As an example, formulations with certain lipidoids, include, but are not limited to, 98N12-5 and may contain 42% lipidoid, 48% cholesterol and 10% poly(ethylene glycol) (PEG) (C14 alkyl chain length). As another example, formulations with certain lipidoids, include, but are not limited to, C12-200 and may contain 50% lipidoid, 10% disteroylphosphatidyl choline, 38.5% cholesterol, and 1.5% poly(ethylene glycol)-dimyristoyl glycerol (DMG) (PEG-DMG).

In some cases, a nucleic acid of the present disclosure is formulated with a lipidoid for systemic intravenous administration can target the liver. For example, a final optimized intravenous formulation comprising a nucleic acid of the present disclosure, and comprising a lipid molar composition of 42% 98N12-5, 48% cholesterol, and 10% PEG-lipid with a final weight ratio of about 7.5 to 1 total lipid to nucleic acid, and a C14 alkyl chain length on the PEG lipid, with a mean particle size of roughly 50-60 nm, can result in the distribution of the formulation to be greater than 90% to the liver.

In another example, an intravenous formulation using a C12-200 lipidoid may have a molar ratio of 50/10/38.5/1.5 of C12-200/disteroylphosphatidyl choline/cholesterol/PEG-DMG, with a weight ratio of 7 to 1 total lipid to nucleic acid, and a mean particle size of 80 nm may be effective to deliver the nucleic acid to hepatocytes. As another example, an MD1 lipidoid-containing formulation may be used to effectively deliver a nucleic acid of the present disclosure to hepatocytes in vivo. The characteristics of optimized lipidoid formulations for intramuscular or subcutaneous routes may vary significantly depending on the target cell type and the ability of formulations to diffuse through the extracellular matrix into the blood stream. While a particle size of less than 150 nm may be desired for effective hepatocyte delivery due to the size of the endothelial fenestrae, use of a lipidoid-formulated nucleic acid of the present disclosure to deliver the formulation to other cells types including, but not limited to, endothelial cells, myeloid cells, and muscle cells may not be similarly size-limited. Different ratios of lipidoids and other components including, but not limited to, disteroylphosphatidyl choline, cholesterol and PEG-DMG, may be used to optimize the formulation of the polynucleotide, primary construct, or mmRNA for delivery to different cell types including, but not limited to, hepatocytes, myeloid cells, muscle cells, etc. For example, the component molar ratio may include, but is not limited to, 50% C12-200, 10% disteroylphosphatidyl choline, 38.5% cholesterol, and %1.5 PEG-DMG. The use of lipidoid formulations for the localized delivery of a nucleic acid of the present disclosure to cells (such as, but not limited to, adipose cells and muscle cells) via either subcutaneous or intramuscular delivery, may not require all of the formulation components desired for systemic delivery, and as such may comprise only the lipidoid and the nucleic acid.

### Lipids, Liposomes, Lipoplexes, and Lipid Nanoparticles

A nucleic acid of the present disclosure can be formulated using one or more lipids, liposomes, lipoplexes, or lipid nanoparticles.

In some cases, a pharmaceutical composition of the present disclosure (comprising a nucleic acid of the present disclosure) includes liposomes. Liposomes are artificially prepared vesicles that may primarily be composed of a lipid bilayer and may be used as a delivery vehicle for the administration of a nucleic acid of the present disclosure. Liposomes can be of various sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may contain a low or a high pH in order to improve the delivery of the pharmaceutical formulations.

The formation of liposomes may depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

In some cases, a pharmaceutical composition of the present disclosure can include, without limitation, liposomes such as those formed from 1,2-dioleyloxy-N,N-dimethylaminopropane (DODMA) liposomes, DiLa2 liposomes from Marina Biotech (Bothell, Wash.), 1,2-dilinoleyloxy-3-dimethylaminopropane (DLin-DMA), 2,2-dilinoleyl-4-(2-dimethylaminoethyl)-[1,3]-dioxolane (DLin-KC2-DMA), and MC3 (US20100324120).

MC3 has the following structure:

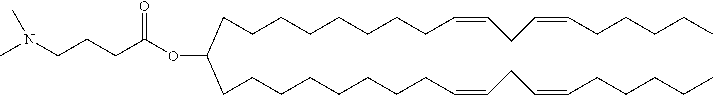 [SMILES: CCCCC/C=C\C/C=C\CCCCCCCCC(CCCCCCCC/C=C\C/C=C\CCCCC)OC(=O)CCCN(C)C]

In one embodiment, pharmaceutical compositions described herein may include, without limitation, liposomes such as those formed from the synthesis of stabilized plasmid-lipid particles (SPLP) or stabilized nucleic acid lipid particle (SNALP) that have been previously described and shown to be suitable for oligonucleotide delivery in vitro and in vivo. The liposome formulations are composed of 3 to 4 lipid components in addition to a nucleic acid of the present disclosure. As an example a liposome can include, but is not limited to, 55% cholesterol, 20% disteroylphosphatidyl choline (DSPC), 10% PEG-S-DSG, and 15% 1,2-dioleyloxy-N,N-dimethylaminopropane (DODMA); and a nucleic acid of the present disclosure. As another example, certain liposome formulations may contain, but are not limited to, 48% cholesterol, 20% DSPC, 2% PEG-c-DMA, and 30% cationic lipid, where the cationic lipid can be 1,2-distearloxy-N,N-dimethylaminopropane (DSDMA), DODMA, DLin-DMA, or 1,2-dilinolenyloxy-3-dimethylaminopropane (DLenDMA); and a nucleic acid of the present disclosure.

In some cases, a composition of the present disclosure comprises one or more cationic lipids. As a non-limiting example, the cationic lipid may be selected from (20Z,23Z)-N,N-dimethylnonacosa-20,23-dien-10-amine, (17Z,20Z)-N,N-dimemylhexacosa-17,20-dien-9-amine, (1Z,19Z)-N5N-dimethylpentacosa-16,19-dien-8-amine, (13Z,16Z)-N,N-dimethyldocosa-13,16-dien-5-amine, (12Z,15Z)-N,N-dimethylhenicosa-12,15-dien-4-amine, (14Z,17Z)-N,N-dimethyltricosa-14,17-dien-6-amine, (15Z,18Z)-N,N-dimethyltetracosa-15,18-dien-7-amine, (18Z,21Z)-N,N-dimethylheptacosa-18,21-dien-10-amine, (15Z,18Z)-N,N-dimethyltetracosa-15,18-dien-5-amine, (14Z,17Z)-N,N-dimethyltricosa-14,17-dien-4-amine, (19Z,22Z)-N,N-dimeihyloctacosa-19,22-dien-9-amine, (18Z,21 Z)-N,N-dimethylheptacosa-18,21-dien-8-amine, (17Z,20Z)-N,N-dimethylhexacosa-17,20-dien-7-amine, (16Z,19Z)-N,N-dimethylpentacosa-16,19-dien-6-amine, (22Z,25Z)-N,N-dimethylhentriaconta-22,25-dien-10-amine, (21 Z,24Z)-N,N-dimethyltriaconta-21,24-dien-9-amine, (18Z)-N,N-dimetylheptacos-18-en-10-amine, (17Z)-N,N-dimethylhexacos-17-en-9-amine, (19Z,22Z)-N,N-dimethyloctacosa-19,22-dien-7-amine, N,N-dimethylheptacosan-10-amine, (20Z,23Z)-N-ethyl-N-methylnonacosa-20,23-dien-10-amine, 1-[(11Z,14Z)-1-nonylicosa-11,14-dien-1-yl]pyrrolidine, (20Z)-N,N-dimethylheptacos-20-en-10-amine, (15Z)-N,N-dimethyl eptacos-15-en-10-amine, (14Z)-N,N-dimethylnonacos-14-en-10-amine, (17Z)-N,N-dimethylnonacos-17-en-10-amine, (24Z)-N,N-dimethyltritriacont-24-en-10-amine, (20Z)-N,N-dimethylnonacos-20-en-10-amine, (22Z)-N,N-dimethylhentriacont-22-en-10-amine, (16Z)-N,N-dimethylpentacos-16-en-8-amine, (12Z,15Z)-N,N-dimethyl-2-nonylhenicosa-12,15-dien-1-amine, (13Z,16Z)-N,N-dimethyl-3-nonyldocosa-13,16-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine,N,N-dimeth-yl-1-[(1S,2S)-2-{[(1R,2R)-2-pentylcyclopropyl]methyl}cyclopropyl]nonadecan-10-amine,N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R-N,N-dimethyl-1-[(9Z,12Z)-octadeca-9,12-dien-1-yloxy]-3-(octyloxy)propan-2-amine, S-N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)-N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azet-idine, (2S)-1-(hexyloxy)-N,N-dimethyl-3-[(9Z,12Z)-octadeca-9,12-dien-1-ylo-xy]propan-2-amine, (2S)-1-(heptyloxy)-N,N-dimethyl-3-[(9Z,12Z)-octadeca-9,12-dien-1-yloxy]pr-opan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-am-ine; (2S)-N,N-dimethyl-1-[(6Z,9Z,12Z)-octadeca-6,9,12-trien-1-yloxy]-3-(oc-tyloxy)propan-2-amine, (2S)-1-[(11Z,14Z)-icosa-11,14-dien-1-yloxy]-N,N-dimethyl-3-(pentyloxy)pro-pan-2-amine, (2S)-1-(hexyloxy)-3-[(11Z,14Z)-icosa-11,14-dien-1-yloxy]-N,N-dimethylprop-an-2-amine, 1-[(11Z,14Z)-icosa-11,14-dien-1-yloxy]-N,N-dimethyl-3-(octyloxy)propan-2--amine, 1-[(13Z,16Z)-docosa-13,16-dien-1-yloxy]-N,N-dimethyl-3-(octyloxy)pr-opan-2-amine, (2S)-1-[(13Z,16Z)-docosa-13,16-dien-1-yloxy]-3-(hexyloxy)-N,N-dimethylpro-pan-2-amine, (2S)-1-[(13Z)-docos-13-en-1-yloxy]-3-(hexyloxy)-N,N-dimethylpropan-2-amin-e, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)-N,N-dimethyl-H(1-metoyloctyl)oxy]-3-[(9Z,12Z)-octadeca-9,12-dien-1-y-loxy]propan-2-amine, (2R)-1-[(3,7-dimethyloctyl)oxy]-N,N-dimethyl-3-[(9Z,12Z)-octadeca-9,12-di-en-1-yloxy]propan-2-amine, N,N-dimethyl-1-(octyloxy)-3-({8-[(1S,2S)-2-{[(1R,2R)-2-pentylcyclopropyl]-methyl}cyclopropyl]octyl}oxy)propan-2-amine, N,N-dimethyl-1-{[8-(2-oclylcyclopropyl)octyl]oxy}-3-(octyloxy)propan-2-amine and (11E,20Z,23Z)-N,N-dimethylnonacosa-11,20,2-trien-10-amine or a pharmaceutically acceptable salt or stereoisomer thereof.

In some cases, an RNA composition of the present disclosure comprises PEG-DMG 2000 (1,2-dimyristoyl-sn-glycero-3-phophoethanolamine-N-[methoxy(polyethylene glycol)-2000). In some cases, an RNA composition of the present disclosure comprises PEG-DMG 2000, DSPC and cholesterol. In some cases, an RNA composition of the present disclosure comprises PEG-DMG 2000, DLin-DMA, DSPC and cholesterol. In some cases, an RNA composition of the present disclosure comprises PEG-DMG 2000, DLin-DMA, DSPC and cholesterol in a molar ratio of 2:40:10:48.

An RNA composition of the present disclosure can include one or more of cholesterol, dipalmitoylphosphatidylcholine (DPCC), phosphatidyl serine (PS), phosphatidylcholine (PC), dimyristoyl glycerol (DMG), 1,2-dilinoleyloxy-N,N-dimethyl-3-aminopropane (DLinDMA), distearoylphosphatidylcholine (DSPC), DMG-poly(ethylene glycol) (DMG-PEG), dioleoyl phosphatidylethanolamine (DOPE), 1,2-di-O-octadecenyl-3-trimethylammonium propane (DOTMA), 1,2-Dioleoyl-sn-glycero-3-phosphocholine (DOPC), distearoylphophatidylethanolamine (DSPE), and N¹,N³,N⁵-tris(3-(didodecylamino)propyl)benzene-1,3,5-tricarboxamide (TT3). In some cases, an RNA composition of the present disclosure comprises cholesterol, DPCC, and PS In some cases, an RNA composition of the present disclosure comprises cholesterol:DPCC:PS at a molar ratio of 5:4:1. In some cases, an RNA composition of the present disclosure comprises PS, PC, and cholesterol. In some cases, an RNA composition of the present disclosure comprises PS:PC:cholesterol in a weight ratio of 1:4.8:2. In some cases, an RNA composition of the present disclosure comprises DLinDMA, DSPC, cholesterol, and DMG-PEG. In some cases, an RNA composition of the present disclosure comprises DOTMA and DOPE. In some cases, an RNA composition of the present disclosure comprises DOTMA:DOPE in a 1:1 molar ratio. In some cases, an RNA composition of the present disclosure comprises DOPC, DOPE, and DSPE-PEG. In some cases, an RNA composition of the present disclosure comprises DOTAP and cholesterol. In some cases, an RNA composition of the present disclosure comprises DOTAP, DOPE, DSPE-PEG-Mannose. In some cases, an RNA composition of the present disclosure comprises TT3, DOPE, cholesterol, and DMG-PEG2000.

In some cases, an RNA composition of the present disclosure comprises one or more cationic lipids. The cationic lipid can be monocationic or polycationic. Cationic lipids typically have a lipophilic moiety, such as a sterol, an acyl or diacyl chain, and have an overall net positive charge. The head group of the lipid typically carries the positive charge. The cationic lipid preferably has a positive charge of 1 to 10 valences, more preferably a positive charge of 1 to 3 valences, and more preferably a positive charge of 1 valence. Examples of cationic lipids include, but are not limited to 1,2-di-O-octadecenyl-3-trimethylammonium propane (DOTMA); dimethyldioctadecylammonium (DDAB); 1,2-dioleoyl-3-trimethylammonium-propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-dimyristoyloxypropyl-1,3-dimethylhydroxyethyl ammonium (DMRIE), and 2,3-dioleoyloxy-N-[2(spermine carboxamide)ethyl]-N,N-dimethyl-1-propanamium trifluoroacetate (DOSPA). Cationic lipids also include lipids with a tertiary amine group, including 1,2-dilinoleyloxy-N,N-dimethyl-3-aminopropane (DLinDMA). Cationic lipids are suitable for formulating RNA in lipid formulations as described herein, such as liposomes, emulsions and lipoplexes. Typically positive charges are contributed by at least one cationic lipid and negative charges are contributed by the RNA. In some cases, the pharmaceutical composition comprises at least one helper lipid, in addition to a cationic lipid. The helper lipid may be a neutral or an anionic lipid. The helper lipid may be a natural lipid, such as a phospholipid, or an analogue of a natural lipid, or a fully synthetic lipid, or lipid-like molecule, with no similarities with natural lipids. In the case where a pharmaceutical composition includes both a cationic lipid and a helper lipid, the molar ratio of the cationic lipid to the neutral lipid can be appropriately determined in view of stability of the formulation and the like.

Cationic lipids suitable for use in a lipid composition of the present disclosure include, but are not limited to, N,N-dioleyl-N,N-dimethylammonium chloride (DODAC), N,N-distearyl-N,N-dimethylammonium bromide (DDAB), N-(1-(2,3-dioleoyloxy) propyl)-N,N,N-trimethylammonium chloride (DOTAP), 1,2-Dioleoyl-3-Dimethylammonium-propane (DODAP), N-(1-(2,3-dioleyloxy)propyl)-N,N,N-trimethylammonium chloride (DOTMA), 1,2-Dioleoylcarbamyl-3-Dimethylammonium-propane (DOCDAP), 1,2-Dilineoyl-3-Dimethylammonium-propane (DLINDAP), dilauryl(C₁₂:0) trimethyl ammonium propane (DLTAP), Dioctadecylamidoglycyl spermine (DOGS), DC-Choi, Dioleoyloxy-N-[2-sperminecarboxamido)ethyl}-N,N-dimethyl-1-propanaminiumt-rifluoroacetate (DOSPA), 1,2-Dimyristyloxypropyl-3-dimethyl-hydroxyethyl ammonium bromide (DMRIE), 3-Dimethylamino-2-(Cholest-5-en-3-beta-oxybutan-4-oxy)-1-(cis,cis-9,12-oc-tadecadienoxy)propane (CLinDMA), N,N-dimethyl-2,3-dioleyloxy)propylamine (DODMA), 2-[5′-(cholest-5-en-3[beta]-oxy)-3′-oxapentoxy)-3-dimethyl-1-(ci-s,cis-9′,12′-octadecadienoxy) propane (CpLinDMA) and N,N-Dimethyl-3,4-dioleyloxybenzylamine (DMOBA), and 1,2-N,N′-Dioleylcarbamyl-3-dimethylaminopropane (DOcarbDAP). In some cases, the cationic lipid is DOTAP or DLTAP.

Neutral lipids suitable for use in a lipid composition of the present disclosure include, for example, a variety of neutral, uncharged or zwitterionic lipids. Examples of neutral phospholipids suitable for use in the present invention include, but are not limited to: 5-heptadecylbenzene-1,3-diol (resorcinol), dipalmitoylphosphatidylcholine (DPPC), distearoylphosphatidylcholine (DSPC), phosphocholine (DOPC), dimyristoylphosphatidylcholine (DMPC), phosphatidylcholine (PLPC), 1,2-distearoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), I-myristoyl-2-palmitoyl phosphatidylcholine (MPPC), I-palmitoyl-2-myristoyl phosphatidylcholine (PMPC), I-palmitoyl-2-stearoyl phosphatidylcholine (PSPC), I,2-diarachidoyl-sn-glycero-3-phosphocholine (DBPC), I-stearoyl-2-palmitoyl phosphatidylcholine (SPPC), I,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine, distearoylphophatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In one embodiment, the neutral phospholipid is selected from the group consisting of distearoylphosphatidylcholine (DSPC) and dimyristoyl phosphatidyl ethanolamine (DMPE).

Anionic lipids suitable for inclusion in a composition of the present disclosure include, but are not limited to, phosphatidylglycerol, cardiolipin, diacylphosphatidylserine, diacylphosphatidic acid, N-dodecanoyl phosphatidyl ethanoloamine, N-succinyl phosphatidylethanolamine, N-glutaryl phosphatidylethanolamine cholesterol hemisuccinate (CHEMS), and lysylphosphatidylglycerol.

“Helper lipids” are lipids that enhance transfection (e.g., transfection of the nanoparticle including the biologically active agent) to some extent. The mechanism by which the helper lipid enhances transfection may include, e.g., enhancing particle stability and/or enhancing membrane fusogenicity. Helper lipids include steroids and alkyl resorcinols. Helper lipids suitable for inclusion in a composition of the present disclosure include, but are not limited to, cholesterol, 5-heptadecylresorcinol, and cholesterol hemisuccinate.

“Stealth lipids” are lipids that increase the length of time for which the nanoparticles can exist in vivo (e.g., in the blood). Stealth lipids suitable for inclusion in a composition of the present disclosure include, but are not limited to, stealth lipids having a hydrophilic head group linked to a lipid moiety.

In some cases, an RNA composition of the present disclosure comprises a heterocyclic lipid. A heterocyclic lipid can include an unsaturated lipid tail, a dihydroimidazole linker and cyclic amine head group.

### Adjuvants

In some cases, an immunogenic composition of the present disclosure comprises an adjuvant (e.g., an immunostimulating amount of an adjuvant). An immunogenic composition of the present disclosure can include an immune-stimulating amount of an adjuvant. Examples of known suitable adjuvants that can be used in humans include, but are not necessarily limited to, alum, aluminum phosphate, aluminum hydroxide, MF59 (4.3% w/v squalene, 0.5% w/v Tween 80™, 0.5% w/v Span 85), CpG-containing nucleic acid (where the cytosine is unmethylated), QS21, monophosphoryl lipid A (MPL), 3-Q-desacyl-4′-monophosphoryl lipid A (3DMPL), extracts from Aquilla, immune-stimulating complexes (ISCOMS; complexes of cholesterol, phospholipids, and Quillaja saponins), LT/CT mutants, poly(D,L-lactide-co-glycolide) (PLG) microparticles, Quil A, interleukins, and the like. For experimental animals, one can use Freund's incomplete adjuvant, or Freund's complete adjuvant. Also suitable for use are N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria: monophosphoryl lipid A, trehalose dimycolate and cell wall skeleton (MPL+TDM+CWS) in a 2% squalene/Tween 80 emulsion. The effectiveness of an adjuvant may be determined by one or more of: i) measuring the amount of antibodies directed against the immunogenic antigen or antigenic epitope thereof; ii) measuring a cytotoxic T lymphocyte response to the antigen; and iii) measuring a helper T cell response to the antigen.

Further exemplary adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59™ (see, e.g., WO 90/14837), containing 5% Squalene, 0.5% Tween 80, and 0.5% Span 85 (optionally containing MTP-PE) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) RIBI™ adjuvant system (RAS), (Ribi Immunochem, Hamilton, Mont.) containing 2% Squalene, 0.2% Tween 80, and one or more bacterial cell wall components such as monophosphorylipid A (MPL), trehalose dimycolate (TDM), and cell wall skeleton (CWS), e.g., MPL+CWS (Detox™); (2) saponin adjuvants, such as QS21 or Stimulon™ (Cambridge Bioscience, Worcester, Mass.; a purified extract of Quillaja saponaria) may be used or particles generated therefrom such as ISCOMs (immunostimulating complexes), which ISCOMS may be devoid of additional detergent e.g. WO 00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3dMPL) e.g. GB-2220221, EP-A-0689454, optionally in the substantial absence of alum when used with pneumococcal saccharides e.g. WO 00/56358; (6) combinations of 3dMPL with, for example, QS21 and/or oil-in-water emulsions (see, e.g. EP-A-0835318, EP-A-0735898, EP-A-0761231); (7) oligonucleotides comprising a CpG motif containing at least one CG dinucleotide, where the cytosine is unmethylated (see, e.g., WO 96/02555, WO 98/16247, WO 98/18810, WO 98/40100, WO 98/55495, WO 98/37919 and WO 98/52581); (8) a polyoxyethylene ether or a polyoxyethylene ester (see, e.g. WO 99/52549); (9) a polyoxyethylene sorbitan ester surfactant in combination with an octoxynol (WO 01/21207) or a polyoxyethylene alkyl ether or ester surfactant in combination with at least one additional non-ionic surfactant such as an octoxynol (WO 01/21152); (10) a saponin and an immunostimulatory oligonucleotide (e.g. a CpG oligonucleotide) (WO 00/62800); (11) an immunostimulant and a particle of metal salt (see, e.g. WO 00/23105); (12) a saponin and an oil-in-water emulsion (see e.g. WO 99/11241); (13) a saponin (e.g. QS21)+3dMPL+IM2 (optionally including a sterol) (see, e.g. WO 98/57659); (14) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutarninyl-L-alanine-2-(1′-2′-dipalmitoyl-sn-glycero-3-hydroxyphosphoryloxy)-ethylamine MTP-PE), etc. Also suitable for use is Matrix-M™; Matrix-M™ is an adjuvant that comprises 40 nm nanoparticles comprising Quillaja saponins, cholesterol, and phospholipid. Adjuvants suitable for administration to a human are of particular interest. In some cases, the adjuvant is one that enhances a CD4+ T helper response to the immunogen. Also suitable for use is a poly inosine:cytosine (poly I:C) nucleic acid. Poly I:C is a synthetic double-stranded RNA Also suitable for use is a cyclic dinucleotide activator of the STING pathway. Examples of suitable cyclic dinucleotide adjuvants include, but are not limited to: 1) bis-(3′,5′)-cyclic dimeric adenosine monophosphate (c-di-AMP); 2) bis-(3′,5′)-cyclic dimeric guanosine monophosphate (c-di-GMP); and bis-(3′,5′)-cyclic dimeric inosine monosphosphate (c-di-IMP). Also suitable for use is poly(I:C).

QS21 has the following structure:

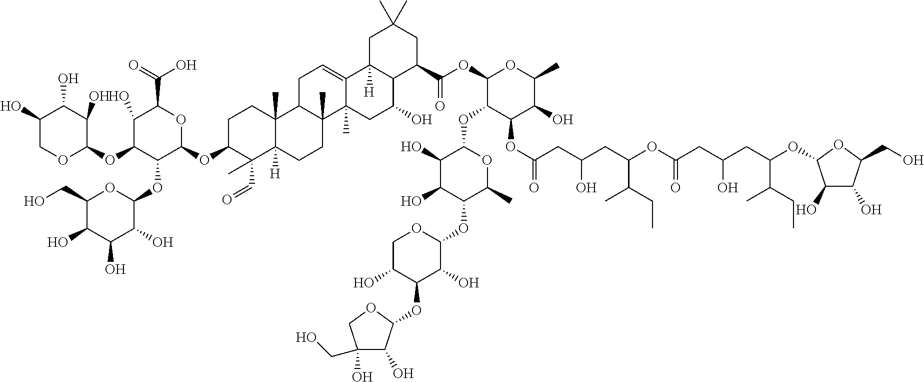

MPL has the following structure:

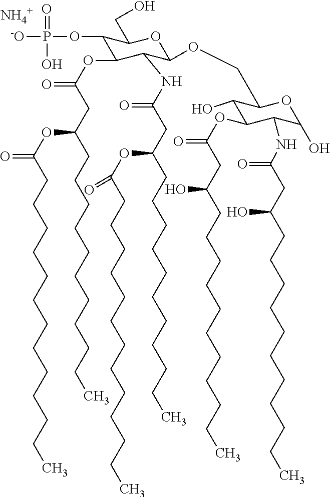

In some instances, the adjuvant is MF59, with or without a CpG-containing oligonucleotide. In other instances, the adjuvant is alum, with or without a CpG-containing oligonucleotide. In other instances, the adjuvant is poly(D,L-lactide-co-glycolide), with or without a CpG-containing oligonucleotide. In other instances, the adjuvant is MPL, with or without a CpG-containing oligonucleotide. In some cases, the adjuvant is Matrix-M™, with or without a CpG-containing oligonucleotide. In some cases, the adjuvant is keyhole limpet hemocyanin. In some cases, the adjuvant is alum. In some cases, the adjuvant is aluminum phosphate. In some cases, the adjuvant is aluminum hydroxide. In some cases, the adjuvant is alum+MPL. In some cases, the adjuvant is MF59. In some cases, the adjuvant is alum+MF59. In some cases, the adjuvant is AS01. AS01 contains QS-21 Stimulon® adjuvant, MPL, and liposomes. In some cases, the adjuvant comprises QS21 and MPL in a liposomal formulation. In some cases, the adjuvant is AS03. A dose of AS03 contains: 10.69 mg squalene; 11.86 mg DL-α-tocopherol; and 4.86 mg polysorbate-80. In some cases, the adjuvant comprises aluminum hydroxide and MPL. In some cases, the adjuvant is AS04. AS04 comprises aluminum hydroxide and MPL. In some cases, the adjuvant is AS15. AS15 is a combination of QS-21 Stimulon® adjuvant, monophosphoryl lipid A, and CpG7909 (an oligonucleotide of the sequence 5′-TCGTCGTTTTGTCGTTTTGTCGTT-3′; (SEQ ID NO:397), in a liposomal formulation. In some instances, the adjuvant is a cyclic dinucleotide (CDN). Suitable CDNs are described below.

In some cases, the adjuvant is selected from the group consisting of an aluminum salt, RIBI, a toll-like receptor agonist, AS01, AS02, AS03, AS04, AS05, a CpG-oligodeoxynucleotide, MF-59, Montanide ISA-51 VG, Montanide ISA-720, Quil A, QS21, a synthetic saponin, an immunostimulatory complex, stearyl tyrosine, a virus-like particle, a reconstituted influenza virosome, a cytokine, mast cell activator compound 48/80, a liposome, a muramyl dipeptide, SAF-1, and combinations thereof. In some cases, the adjuvant is selected from the group consisting of an aluminum salt, alum, PHAD, a CDN, AS01, AS04, a CpG oligodeoxynucleotide, MF59, and combinations of two or more of the foregoing.

In some cases, the adjuvant is a disaccharide synthetic lipid compound, e.g., as described in U.S. Pat. No. 9,518,078. A disaccharide synthetic lipid compound can be a phosphorylated hexaacyl disaccharide (PHAD).

In some cases, the adjuvant is a PHAD of the following structure:

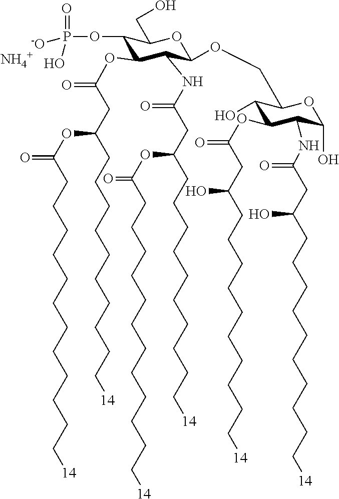

In some cases, a disaccharide synthetic lipid compound is a compound referred to as MPLA-B in U.S. Pat. No. 9,518,078; and has the following structure:

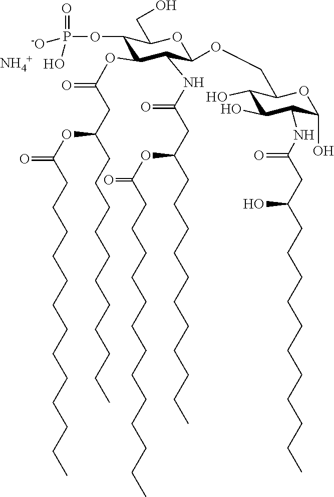

In some cases, a disaccharide synthetic lipid compound is a compound referred to as MPLA-D in U.S. Pat. No. 9,518,078; and has the following structure:

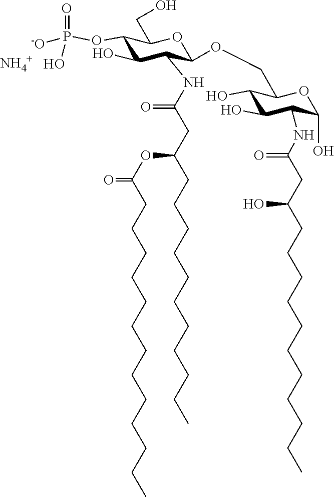

In some cases, a disaccharide synthetic lipid compound is a compound referred to as MPLA-C in U.S. Pat. No. 9,518,078; and has the following structure:

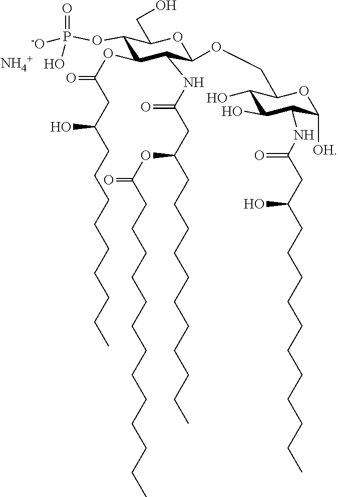

In some cases, the adjuvant is PHAD (also referred to as a glucopyranosyl lipid adjuvant (GLA)). In some cases, the adjuvant is a combination of alum and a PHAD.

### Cyclic Dinucleotides

In some cases, an immunogenic composition of the present disclosure comprises a cyclic dinucleotide (CDN).

In some cases, a CDN suitable for use in an immunogenic composition of the present disclosure is of Formula (I):

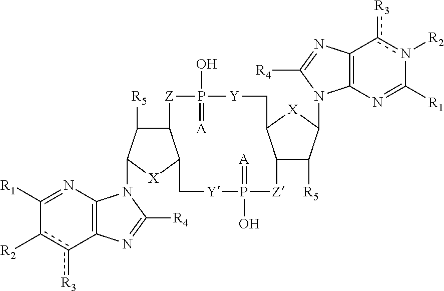

wherein:
A is S or O;
X is S, N, O, CH₂;
Y, Y′ is NH, CH₂, O;
Z, Z′ is NH, CH₂, O;
R1 represents hydrogen or NH₂ which may be substituted;
R2 is hydrogen or absent;
R3 represents NH₂, O, OH, H, or a halogen;
R4 represents hydrogen, halogen, or a straight or branched C₁-C₆ alkyl group which may optionally be substituted;
R5 represents hydrogen, OH or a straight or branched C₁-C₆ alkyl chain or C₁-C₆ straight or branched alkoxy chain which may optionally be substituted;
[symbol] is a single or double bond;
or conjugates thereof, and salts or solvates thereof. See, e.g., US 2008/0286296.

In formula (I), the purine residue is in some cases a guanine (G), adenine (A), xanthine or hypoxanthine (X), or inosine (I) residue. The compound can have identical purine residues, e.g. c-diGMP, c-diAMP, c-diIMP, or c-dXMP, or can contain different purine residues, e.g. c-GpAp, c-GpIp, c-GpXp, c-ApIp, c-ApXp, or c-IpXp. Further, R5 is in some cases an OH group. In addition, X is in some cases an oxygen atom. In one embodiment, Y, Y′, Z, and Z′ are an oxygen atom, O. Thus, in one embodiment, the compound of formula (I) is a cyclic bis(3′-5′)diguanylic acid (c-diGMP) or conjugates thereof or a cyclic bis(3′-5′)diadenylic acid (c-diAMP) or conjugates thereof, or salts or solvates thereof. In one embodiment, the compound of formula (I) is cyclic Bis(3′-5′)adenylic acid, which is also referred to as c-di-AMP; or the pegylated conjugate. With the term “which may be substituted” is meant the substitution with a straight or branched C1-C6 alkyl group or a straight or branched C1-C6 alkoxy group and/or with a halogen, hydroxyl group or carboxyl group.

In some cases, a CDN suitable for use in an immunogenic composition of the present disclosure is selected from the group consisting of cyclic di-adenosine monophosphate (c-di-AMP), cyclic di-guanosine monophosphate (c-di-GMP), and cyclic guanosine monophosphate-adenosine monophosphate (cGAMP). In some cases, a CDN suitable for use in an immunogenic composition of the present disclosure is cGAMP (2′-3′-cyclic GMP-AMP) or cGAMP (3′-3′-cyclic GMP-AMP). In some cases, a CDN suitable for use in an immunogenic composition of the present disclosure is cGAMP (2′-3′-cyclic GMP-AMP). In some cases, a CDN suitable for use in an immunogenic composition of the present disclosure is cGAMP (3′-3′-cyclic GMP-AMP).

In some cases, a CDN suitable for use in an immunogenic composition of the present disclosure is of Formula (II):

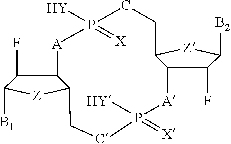

where:
A, C, A′ and C′ are independently selected from NH, O, and S;
X, Y, X′, and Y′ are independently selected from O or S;
Z and Z′ are independently selected from O, S, NH, and CH₂; and
B₁ and B₂ are independently a purine selected from:

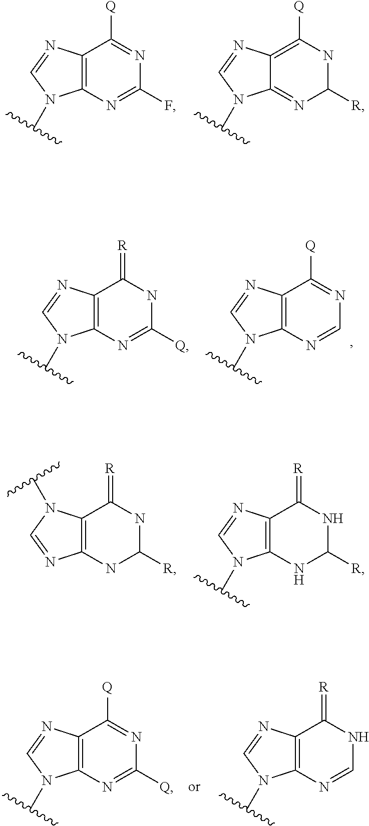

where:
Q is hydrogen or NH₂;
Nitrogen is optionally substituted with a C₁-C₆ alkyl or a C₁-C₆ acyl group; and
R is O or S.

In some cases, a CDN suitable for inclusion in an immunogenic composition of the present disclosure is a fluorinated CND. In some cases, the fluorinated CDN is 2′-F-c-diGMP having the following structure:

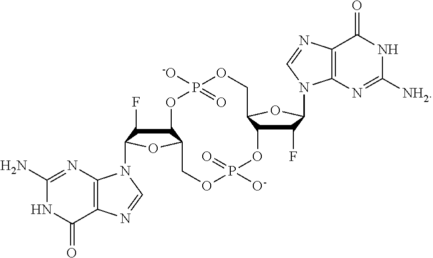

### Archaeal Glycolipid

In some cases, an immunogenic composition of the present disclosure comprises an archaeosome. For example, in some cases, an immunogenic composition of the present disclosure comprises an archaeosome comprising at least one polar synthetic lipid, where the at least one polar synthetic lipid comprises at least one carbohydrate or anionic group linked by covalent bonding to at least one free hydroxyl group of an archaeal core lipid.

An archaeal lipid suitable for use in an immunogenic composition of the present disclosure comprises a polar lipid based on a 2, 3-dialkylglycerol skeleton. These 2, 3-dialkylglycerol groups are isoprenoid and the simplest molecules are derivatives or 2,3-dibiphytanyl-O-sn-glycerol (archeol); for instance, two isoprenoid units of 20 carbons joined at positions sn-2 and sn-3 of glycerol. These alkyl chains are generally saturated; nevertheless, some forms have double bonds in different positions. These lipids have one or two groups of polar head, which may be different with units 2, 3-sn-glycerol joined by C40 alkyl components which are also isoprenoid molecules. For instance, calarcheol (so called because it is the predominant form in some thermophile archaebacteria), has two C40 isoprenoid units bonded from positions 2 to 3′ and from position 3 to 2′.

In some cases, an archaeal adjuvant suitable for use in an immunogenic composition of the present disclosure comprises multivalent cations in association with aggregates of a plurality of spherical archaeal polar lipid structures containing aqueous compartments (e.g., an “AMVAD structure”), where the archaeal polar lipid is a total polar lipids extract or archaetidyl glycerophosphate-O-methyl, obtained from an archaeal species. The multivalent cations can be divalent or trivalent cations. The multivalent cations can be divalent Ca²⁺ or Mg², or trivalent Al³⁺. The Ca²⁺ can be provided as CaCl₂. The Al³⁺ can be provided as AlCl₃ or AlK(SO₄)₂. In some cases, the total polar lipids extract from an archaeal species is mixed with neutral lipids from the archaeal species. See, e.g., U.S. Patent Publication No. 2013/0195932.

In some cases, lipids suitable for use in an immunogenic composition of the present disclosure comprises 1,2-di-O-hexadecyl-sn-glycero-3-phosphatidylcholine and 1,2-di-O-phytanyl-sn-glycero-3-phosphatidylethanolamine. In some cases, the 1,2-di-O-hexadecyl-sn-glycero-3-phosphatidylcholine and 1,2-di-O-phytanyl-sn-glycero-3-phosphatidylethanolamine form uniformly sized particles; for example, the particles can comprise: liposomes, nanoliposomes, niosomes, microspheres, nanospheres, nanoparticles, micelles or archaeosomes.

In some cases, an archaeosome comprises at least one polar synthetic lipid, where the at least one polar synthetic lipid comprises at least one carbohydrate or anionic group linked by covalent bonding to at least one free hydroxyl group of an archaeal core lipid. In some cases, the archaeal core lipid is archaeol (2,3-di-O-diphytanyl-sn-glycerol). In some cases, the archaeal core lipid is caldarchaeol (2,2′,3,3′-tetra-O-dibiphytanyl-sn-diglycerol). In some cases, the the carbohydrate group is selected from the group consisting of: β-D-Glc-(1,6)-β-D-Glc-; β-D-Glc-(1,6)-α-D-Glc-; α-D-Glc-(1,6)-β-D-Glc-; β-D-Glc-(1,4)-β-D-Glc-; α-D-Glc-(1,4)-β-D-Glc-; β-D-Gal-(1,4)-β-D-Glc-; α-D-Gal-(1,6)-β-D-Glc-; β-D-Glc-(1,6)-β-D-Glc-(1,6)-β-D-Glc-; α-D-Glc-(1,4)-α-D-Glc-(1,4)-β-D-Glc-; α-D-Man-(1,2)-α-D-Man-(1,2)-α-D-Man-; and α-D-Man-(1,2)-α-D-Man-(1,2)-α-D-Man-(1,2)-α-D-Man-. In some cases, the carbohydrate group comprises two or three β-D-Glc-units in (1,6) linkage. In some cases, the carbohydrate group is a Galactose-Glucose (gal-glc) group. In some cases, the anionic group is selected from the group consisting of phosphoserine, phosphoethanolamine, phosphoinositol and phosphoglycerol. In some cases, the at least one anionic lipid is selected from the group consisting of archaetidylglycerol, archaetidylglycerolphosphate-methyl, archaetidylserine, and archaetidylinositol. In some cases, the archaeosome comprises at least one conventional lipid. In some cases, the at least one conventional lipid is selected from a group consisting of phosphatidylglycerol, phosphatidylserine, sulfoquinovosyl diacylglycerol (SQDG), and cholesterol. In some cases, the at least one conventional lipid comprises cholesterol, and wherein cholesterol is present in an amount of between 10 and 45 mol % of the total lipid composition. In some cases, the phosphatidylglycerol is present in an amount of between 20 and 80 mol % of the lipid composition. In some cases, the phosphatidylserine is present in an amount of between 10 and 30 mol % of the lipid composition. In some cases, the at least one polar synthetic lipid comprises at least one synthetic immunoactive glycolipid and at least one anionic lipid, and the archaeosome further comprises at least one stabilizing lipid. In some cases, the at least one polar synthetic lipid comprises caldarchaeol having one carbohydrate head group and one anionic head group. In some cases, the carbohydrate head group comprises gentiobiose and the anionic head group comprises phosphoinositol. In some cases, the at least one polar synthetic lipid comprises a first caldarchaeol having two carbohydrate head groups and a second caldarchaeol having two anionic head groups, and wherein the at least one stabilizing lipid is the first and/or second caldarchaeol. In some cases, the at least one polar synthetic lipid comprises gentiotriose-archaeol and wherein the at least one stabilizing lipid comprises cholesterol and at least one of phosphatidylethanolamine, archaetidylglycerol, archaetidylserine or archaetidylglycerolphosphate-methyl.

Caldarchaeol is also known as dibiphytanyldiglycerol tetraether. Two glycerol units are linked together by two strains that consist of two phytanes linked together to form a linear chain of 32 carbon atoms. Caldarchaeol has the following structure:

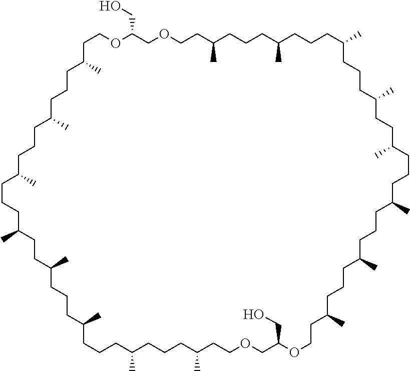 [SMILES: C[C@@H]1CCC[C@H](C)CCC[C@H](C)CC[C@@H](C)CCC[C@@H](C)CCC[C@@H](C)CCC[C@@H](C)CCO[C@H](CO)COCC[C@H](C)CCC[C@H](C)CCC[C@H](C)CCC[C@H](C)CC[C@@H](C)CCC[C@@H](C)CCC[C@@H](C)CCC[C@@H](C)CCO[C@H](CO)COCC[C@H](C)CCC1]

Archaeal lipids can be obtained from any archaea of the phyla Euryarchaeota, Crenarchaeota, Korarchaeota, or, Nanoarchaea. Archaeal lipids can be obtained from any archaea of the genus Thermococcus, Sulfolobus, Halobacterium, Methanococcus, Ferroglobus, Thermoplasma, Archaeoglobus, Haloquadratum, or Halorubrum. Suitable sources of archaeal lipids include, but are not limited to, Thermus aquaticus, Thermus thermophilus; Methanobrevibacter smithii; Thermoplasma acidophilum; a Sulfolobus species, e.g. Sulfolobus acidocaldarius, Sulfolobus solfataricus, Sulfolobus islandicus, Sulfolobus tokodaii, etc.; a Pyrobaculum species, e.g. Pyrobaculum islandicum or Pyrobaculum aerophilum; a Methanococcus species, e.g., Methanocaldococcus vulcanius, Methanocaldococcus jannaschii, Methanococcus acolicus, Methanococcus voltae; or a Halobacterium species such as Halobacterium salinarum; Methanopyrus kandleri; Methanobacterium espanolae; Methanosphaera stadtmanae; Methanosarcina mazei; Natronobacterium magadii; etc.

Total polar lipids (TPL) can be extracted from archaea and collected as the acetone-insoluble fraction. Choquet et al. (1994) Appl. Microbiol. Biotechnol. 42:375; Bligh and Dyer (1959) Can. J. Biochem. Physiol. 37:911. The polar lipids consist of regularly branched, and usually fully saturated, phytanyl chains of 20, 25, or 40 carbon length, with the 20 and 40 being most common. Archaeosomes can be prepared by hydrating TPL in a buffer (e.g., phosphate-buffered saline). The TPL-buffer solution can be sonicated (e.g., at 60 Hz for 10 min).

TPL can be extracted from archaea by stirring the cells (which may be lyophilized) with chloroform-methanol (2:1, v/v) for 1 hour at room temperature. The suspension is passed through a sintered glass filter, and the residue reextracted for an additional hour. Combined filtrates are evaporated, taken up in chloroform-methanol-water (60:30:4.5, v/v/v), and passed through Sephadex G-25 for removal of nonlipid contaminations. Langworthy et al. (1977) J. Bacteriol. 130:1326.

The mean diameter of archaeosomes in an archaeosomal formulation can range from about 50 nm to 600 nm, e.g., from 50 nm to 100 nm, from 100 nm to 150 nm, from 150 nm to 200 nm, from 200 nm to 250 nm, from 250 nm to 300 nm, from 300 nm to 400 nm, from 400 nm to 450 nm, from 450 nm to 500 nm, from 500 nm to 550 nm, or from 550 nm to 600 nm.

### Containers

The present disclosure provides a container comprising an immunogenic composition of the present disclosure. The container can be sterile. The immunogenic composition can be sterile. The immunogenic composition can be suitable for administration to a human subject; e.g., the immunogenic composition can be free of pyrogens, allergens, or other substances that may be harmful to a human subject. Suitable containers include unit-dose containers, multi-dose sealed containers, ampules, vials, syringes, and the like. In some cases, the container is a syringe.

### Methods of Inducing an Immune Response

The present disclosure provides a method of inducing an immune response (e.g., a protective immune response) to at least one HCV genotype in a mammalian subject. In some cases, a method of the present disclosure for inducing an immune response in an individual to at least one HCV genotype comprises administering a composition of the present disclosure, where the composition comprises one or more RNA molecules encoding one or more immunogens (e.g., HCV E1/E2; HCV E1; HCV E2; T-cell epitope polypeptide(s); fusion polypeptides).

### Prime-Boost

In some cases, a prime-boost vaccine protocol is used. In some cases, a first (priming) composition (“first dose”) is administered, where the first composition comprises one or more RNA molecules comprising nucleotide sequences encoding one or more of: a) an HCV E1/E2 heterodimer; b) an HCV E2 polypeptide; c) an HCV E1 polypeptide; d) an HCV Core polypeptide; e) an HCV Core-E1-E2 polyprotein; and f) a T-cell epitope polypeptide(s) (or fusion polypeptide comprising two or more T-cell epitope polypeptides); and, after a time, a second (booster) composition (“second dose”) is administered, where the second immunogenic comprises one or more RNA molecules comprising nucleotide sequences encoding one or more of a) an HCV E1/E2 heterodimer; b) an HCV E2 polypeptide; c) an HCV E1 polypeptide; d) an HCV Core polypeptide; e) an HCV Core-E1-E2 polyprotein; and f) a T-cell epitope polypeptide(s) (or fusion polypeptide comprising two or more T-cell epitope polypeptides). The second composition can be administered at a time period of from 1 day to 1 year following administration of the first composition. For example, a second immunogenic composition can be administered at a time period of from 1 day to 1 week, from 1 week to 2 weeks, from 2 weeks 3 weeks, from 3 weeks to 1 month, from 1 month to 2 months, from 2 months to 6 months, or from 6 months to 1 year following administration of the first composition. As an example, a second immunogenic composition (“second dose”) can be administered at a time period of from 3 weeks to 20 weeks (e.g., 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, or 20 weeks) following administration of the first composition (“first dose”). In some cases, the first and the second compositions are administered via the same route of administration. In some cases, the first and the second compositions are administered via different routes of administration. For example, in some cases, the first composition is administered via an intranasal route of administration; and the second composition is administered via an intramuscular route of administration. As another example, in some cases, the first composition is administered via an intramuscular route of administration; and the second composition is administered via an intranasal route of administration. In some cases, at least a third dose of a composition of the present disclosure is administered at a third time. A third immunogenic composition (“third dose”) can be administered at a time period of from 15 weeks to about 40 weeks (e.g., from 15 weeks to 32 weeks, from 16 weeks to 32 weeks, from 15 weeks to 20 weeks, from 16 weeks to 24 weeks, from 24 weeks to 32 weeks, from 24 weeks to 40 weeks) after the second dose.

As another example of a prime-boost vaccine protocol, in some cases, a first (priming) composition is administered, where the first composition comprises one or more RNA molecules comprising nucleotide sequences encoding one or more of: a) an HCV E1/E2 heterodimer; b) an HCV E2 polypeptide; c) an HCV E1 polypeptide; and d) a T-cell epitope polypeptide(s) (or fusion polypeptide comprising two or more T-cell epitope polypeptides); and, after a time, a second (booster) composition is administered, where the second immunogenic comprises one or more of a) an HCV E1/E2 heterodimer; b) an HCV E2 polypeptide; c) an HCV E1 polypeptide; and d) a T-cell epitope polypeptide(s) (or fusion polypeptide comprising two or more T-cell epitope polypeptides). The second composition can be administered at a time period of from 1 day to 1 year following administration of the first composition. For example, a second immunogenic composition can be administered at a time period of from 1 day to 1 week, from 1 week to 2 weeks, from 2 weeks 3 weeks, from 3 weeks to 1 month, from 1 month to 2 months, from 2 months to 6 months, or from 6 months to 1 year following administration of the first composition. In some cases, the first and the second compositions are administered via the same route of administration. In some cases, the first and the second compositions are administered via different routes of administration. For example, in some cases, the first composition is administered via an intranasal route of administration; and the second composition is administered via an intramuscular route of administration. As another example, in some cases, the first composition is administered via an intramuscular route of administration; and the second composition is administered via an intranasal route of administration.

As another example of a prime-boost vaccine protocol, in some cases, a first (priming) composition is administered, where the first composition comprises one or more of a) an HCV E1/E2 heterodimer; b) an HCV E2 polypeptide; c) an HCV E1 polypeptide; and d) a T-cell epitope polypeptide(s) (or fusion polypeptide comprising two or more T-cell epitope polypeptides); and, after a time, a second (booster) composition is administered, where the second immunogenic comprises one or more RNA molecules comprising nucleotide sequences encoding one or more of: a) an HCV E1/E2 heterodimer; b) an HCV E2 polypeptide; c) an HCV E1 polypeptide; and d) a T-cell epitope polypeptide(s) (or fusion polypeptide comprising two or more T-cell epitope polypeptides). The second composition can be administered at a time period of from 1 day to 1 year following administration of the first composition. For example, a second immunogenic composition can be administered at a time period of from 1 day to 1 week, from 1 week to 2 weeks, from 2 weeks 3 weeks, from 3 weeks to 1 month, from 1 month to 2 months, from 2 months to 6 months, or from 6 months to 1 year following administration of the first composition. In some cases, the first and the second compositions are administered via the same route of administration. In some cases, the first and the second compositions are administered via different routes of administration. For example, in some cases, the first composition is administered via an intranasal route of administration; and the second composition is administered via an intramuscular route of administration. As another example, in some cases, the first composition is administered via an intramuscular route of administration; and the second composition is administered via an intranasal route of administration.

In some cases, a prime-boost vaccine protocol comprises: i) administering a first compositions, where the first composition comprises one or more RNA molecules comprising nucleotide sequences encoding one or more of: a) an HCV E1/E2 heterodimer; b) an HCV E2 polypeptide; c) an HCV E1 polypeptide, where the HCV E1 and/or E2 polypeptide is of a first genotype; and ii) and, after a time, administering a second (booster) composition, where the second immunogenic comprises one or more RNA molecules comprising nucleotide sequences encoding one or more of: a) an HCV E1/E2 heterodimer; b) an HCV E2 polypeptide; c) an HCV E1 polypeptide, where the HCV E1 and/or E2 polypeptide is of a second genotype. For example, the first genotype can be genotype 1; and the second genotype can be genotype 3. As another example, the first genotype can be genotype 1; and the second genotype can be genotype 2. As another example, the first genotype can be genotype 3; and the second genotype can be genotype 1.

In some cases, a prime-boost regimen comprises: a) administering a composition comprising an RNA molecule(s) of the present disclosure; and b) administering a polypeptide (e.g., a TP polypeptide; a TP fusion polypeptide).

### General Considerations

A composition of the present disclosure is generally administered to a human subject who: i) has an HCV infection; or ii) is at risk of acquiring an HCV infection (e.g., is at greater risk than the general population of acquiring an HCV infection); or iii) is naïve with respect to HCV infection, so as to prevent or at least partially arrest the development of disease and its complications. An amount adequate to accomplish this is defined as a “therapeutically effective dose” or a “therapeutically effective amount.” “Prophylactic” use of a subject composition generally refers to administration to an individual who has not been infected with HCV (e.g., a “naïve” individual). “Therapeutic” use of a subject composition can refer to “prophylactic” use (administration to an individual who has not been infected with HCV) and/or to administration to an individual who has an HCV infection. A “therapeutically effective amount” of a composition of the present disclosure, can be an amount that, when administered in one or more doses to an individual who is not infected with HCV, is effective to induce an immune response in the individual to HCV. A “therapeutically effective amount” of a composition of the present disclosure, can be an amount that, when administered in one or more doses to an individual who is infected with HCV, is effective to enhance an immune response in the individual to HCV.

Amounts effective for therapeutic use will depend on, e.g., the manner of administration, the weight and general state of health of the patient, and the judgment of the prescribing physician. Single or multiple doses of a subject immunogenic composition can be administered depending on the dosage and frequency required and tolerated by the patient, and route of administration.

In some cases, an effective amount of a composition of the present disclosure is an amount that, when administered to an individual in one or more doses, is effective to induce an antibody response (e.g., a neutralizing antibody response) to HCV in the individual. For example, antibody to HCV (e.g., extracellular HCV), and/or to an HCV-infected cell, can be induced.

An effective amount of a composition of the present disclosure can be an amount that, when administered to an individual in one or more doses, is effective to induce a neutralizing antibody response to HCV of a variety of genotypes (e.g., genotype 1; genotype 3; etc.). A neutralizing antibody response reduces binding of HCV to one or more host receptors for HCV and inhibits entry of HCV into a cell.

In some cases, an effective amount (e.g., a therapeutically effective amount) of a composition of the present disclosure is an amount that, when administered to an individual in one or more doses, is effective to induce a cytotoxic T lymphocyte (CTL) response to HCV. For example, a CTL response to an HCV-infected cell can be induced.

In some cases, an effective amount (e.g., a therapeutically effective amount) of a composition of the present disclosure is an amount that, when administered to an individual in one or more doses, is effective to induce a helper T lymphocyte (e.g., CD4+ T cell) to HCV in an individual.

In some cases, an effective amount (e.g., a therapeutically effective amount) of a composition of the present disclosure is an amount that, when administered to an individual in one or more doses, is effective to induce an antibody response (e.g., a neutralizing antibody response) and/or a CTL response and/or a helper T cell response to HCV genotype 1. In some cases, an effective amount (e.g., a therapeutically effective amount) of a composition of the present disclosure is an amount that, when administered to an individual in one or more doses, is effective to induce an antibody response (e.g., a neutralizing antibody response) and/or a CTL response and/or a helper T cell response to HCV genotype 3. In some cases, an effective amount (e.g., a therapeutically effective amount) of a composition of the present disclosure is an amount that, when administered to an individual in one or more doses, is effective to induce an antibody response (e.g., a neutralizing antibody response) and/or a CTL response and/or a helper T cell response to HCV genotype 1 and HCV genotype 3. In some cases, an effective amount (e.g., a therapeutically effective amount) of a composition of the present disclosure is an amount that, when administered to an individual in one or more doses, is effective to induce an antibody response (e.g., a neutralizing antibody response) and/or a CTL response and/or a helper T cell response to HCV of any genotype.

A composition of the present disclosure is generally administered in an amount effective to elicit an immune response, e.g., a humoral immune response (e.g., an antibody response) and/or a CTL response, in the mammalian subject.

Suitable doses include from about 0.5 μg to about 100 μg. For example, suitable doses include from about 0.5 μg to about 1.0 μg, from about 1 μg to about 5 μg, from about 5 μg to about 10 g, from about 10 μg to about 15 μg, from about 15 μg to about 20 μg, from about 20 μg to about 25 μg, from about 25 μg to about 30 μg, from about 30 μg to about 40 μg, from about 40 μg to about 50 μg, from about 50 μg to about 60 μg, from about 60 μg to about 70 μg, from about 70 μg to about 80 μg, from about 80 μg to about 90 μg, or from about 90 μg to about 100 μg. Suitable doses include from about 0.5 μg to about 5 μg, from about 5 μg to about 25 μg, from about 25 μg to about 50 μg, from about 50 g to about 75 μg, or from about 75 μg to about 100 μg. Multiple doses may be administered.

The initial administration can be followed by booster immunization of the same composition or a different composition. In some instances, a subject method of inducing an immune response involves an initial administration of a composition of the present disclosure, followed by at least one booster, and in some instances involves two or more (e.g., three, four, or five) boosters. The interval between an initial administration and a booster, or between a give booster and a subsequent booster, can be from about 1 week to about 12 weeks, e.g., from about 1 week to about 2 weeks, from about 2 weeks to about 4 weeks, from about 4 weeks to about 6 weeks, from about 6 weeks to about 8 weeks, from about 8 weeks to about 10 weeks, or from about 10 weeks to about 12 weeks. The interval between an initial administration and a booster, or between a give booster and a subsequent booster, can be from 4 months to 6 months, or from 6 months to 1 year.

In general, immunization can be accomplished by administration of a composition of the present disclosure by any suitable route, including administration of the composition orally, nasally, nasopharyngeally, parenterally, enterically, gastrically, topically, transdermally, subcutaneously, intramuscularly, in tablet, solid, powdered, liquid, aerosol form, locally or systemically, with or without added excipients. Actual methods for preparing parenterally administrable compositions will be known or apparent to those skilled in the art and are described in more detail in such publications as Remington's Pharmaceutical Science, 15th ed., Mack Publishing Company, Easton, Pa. (1980). In some instances, immunization is accomplished by intramuscular injection of an immunogenic composition of the present disclosure.

### Individuals Suitable for Administration

Individuals who are suitable for administration with a composition of the present disclosure include immunologically naïve individuals (e.g., individuals who have not been infected with HCV and/or who have not been administered with an HCV vaccine). Individuals suitable for administration include humans.

Individuals who are suitable for administration with a composition of the present disclosure composition of the present disclosure include individuals who are at greater risk than the general population of becoming infected with HCV, where such individuals include, e.g., intravenous drug users; individuals who are the recipients, or the prospective recipients, of blood or blood products from another (donor) individual(s); individuals who are the recipients, or the prospective recipients, of non-autologous cells, tissues, or organs from another (donor) individual; health care workers; emergency medical and non-medical personnel (e.g., first responders; fire fighters; emergency medical team personnel; etc.) and the like.

Individuals who are suitable for administration with a composition of the present disclosure composition of the present disclosure include individuals who recently became exposed to HCV or who recently became infected with HCV. For example, a subject composition can be administered to an individual within from about 24 hours to about 48 hours, from about 48 hours to about 1 week, or from about 1 week to about 4 weeks, following possible or suspected exposure to HCV or following infection with HCV.

Individuals who are suitable for administration with a composition of the present disclosure composition of the present disclosure include individuals who were previously infected with HCV, who were treated for HCV, and who were cured.

Individuals who are suitable for administration with a composition of the present disclosure composition of the present disclosure include individuals who have been diagnosed as having an HCV infection, and include chronically infected individuals. In some cases, an individual who has been diagnosed as having an HCV infection is treated with an anti-viral agent and a composition of the present disclosure. Suitable anti-viral agents for treating HCV infection include, e.g., ribavirin (1-β-D-ribofuranosyl-1H-1,2,4-triazole-3-carboxamide); interferon-alpha (IFN-α) (where “IFN-α” includes IFN-α2α; IFN-α2b; IFN-α that is conjugated with poly(ethylene glycol) (“pegylated IFN-α), where the pegylated IFN-α can be pegylated IFN-α2α or pegylated IFN-(α 2b); an HCV NS3 protease inhibitor (e.g., boceprevir; telaprevir); and an HCV NS5 protease inhibitor.

In some cases, an individual who has been diagnosed as having an HCV infection is treated with, e.g.: 1) IFN-(α+ribavirin; and a composition of the present disclosure; 2) IFN-(α+ribavirin+an HCV protease inhibitor (e.g., boceprevir or telaprevir); and an immunogenic composition of the present disclosure; 3) Harvoni; and an immunogenic composition of the present disclosure; 4) an inhibitor of HCV NS5B; and an immunogenic composition of the present disclosure; 5) an inhibitor of HCV NS5A; and an immunogenic composition of the present disclosure; or 6) an inhibitor of HCV NS5B+an inhibitor of HCV NS5A; and an immunogenic composition of the present disclosure. Suitable anti-viral agents for treating HCV infection include Sovaldi (Sofosbuvir; a nucleotide analog that functions as an NS5B polymerase inhibitor), alone or in combination with pegylated IFN-α and ribavirin; and Harvoni. Harvoni is a formulation comprising 90 mg ledipasvir and 400 mg sofosbuvir. Ledipasvir is an inhibitor of HCV NS5A.

### Examples of Non-Limiting Aspects of the Disclosure

### Aspects Set A

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A composition comprising: a) one or more RNA molecules, wherein the one or more RNA molecules comprises: i) at least one 5′ cap structure and/or a 5′ untranslated region (5′ UTR) and/or a 5′ internal ribosome entry site (IRES); ii) a polypeptide-encoding region comprising one or more nucleotide sequences encoding an hepatitis C virus (HCV) E1 polypeptide and/or an HCV E2 polypeptide; and b) one or more of: i) a lipid; ii) a polymer; iii) a peptide; iv) a viral-like particle; and v) a cationic nanoemulsion.

Aspect 2. The composition of aspect 1, wherein the one or more RNA molecules comprises a 5′ cap structure and a 5′ UTR, wherein the 5′UTR is 3′ of the 5′ cap structure.

Aspect 3. The composition of aspect 1 or aspect 2, wherein the one or more RNA molecules comprises a 3′ UTR and/or a 3′ tailing sequence.

Aspect 4. The composition of aspect 3, wherein the 3′ tailing sequence is a poly(adenosine) (poly-A) sequence.

Aspect 5. The composition of any one of aspects 1-4, wherein the one or more RNA molecules include at least one 5′ cap structure, and wherein the at least one 5′ cap structure is selected from the group consisting of Cap0, Cap1, ARCA, inosine, N1-methyl-guanosine, 2′fluoro-guanosine, 7-deaza-guanosine, 8-oxo-guanosine, 2-amino-guanosine, LNA-guanosine, and 2-azido-guanosine.

Aspect 6. The composition of any one of aspects 1-5, wherein the one or more RNA molecules comprises one or more of: a nucleoside base modification, a sugar modification, and a backbone modification.

Aspect 7. The composition any one of aspects 1-6, wherein at least one of the one or more RNA molecules comprises a nucleotide of Formula I:

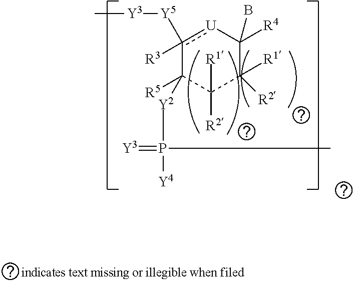

wherein:
U O, S, N(R^{U})ₙᵤ, or C(R^{U})ₙᵤ, where nu is an integer nu is an integer from 0 to 2 and each R^{U} is, independently, H, halo, or optionally substituted alkyl;
[symbol] is a single or double bond;
[symbol] is a single bond or absent;
each of R^{1′}, R^{1″}, R^{2″}, R³, R⁴, and R⁵ is, independently, H, halo, hydroxy, thiol, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, or absent; wherein the combination of R³ with one or more of R^{1′}, R^{1″}, R^{2′}, R^{2″}, or R⁵ can join together to form optionally substituted alkylene or optionally substituted heteroalkylene and, taken together with the carbons to which they are attached, provide an optionally substituted heterocyclyl; wherein the combination of R⁵ with one or more of R^{1′}, R^{1″}, R^{2′}, or R^{2″} can join together to form optionally substituted alkylene or optionally substituted heteroalkylene and, taken together with the carbons to which they are attached, provide an optionally substituted heterocyclyl; and wherein the combination of R⁴ and one or more of R^{1′}, R^{1″}, R^{2′}, R^{2″}, R³, or R⁵ can join together to form optionally substituted alkylene or optionally substituted heteroalkylene and, taken together with the carbons to which they are attached, provide an optionally substituted heterocyclyl;
each of Y¹, Y², and Y³, is, independently, O, S, —NR^{N1}—, optionally substituted alkylene, or optionally substituted heteroalkylene, wherein R^{N1} is H, optionally substituted alkyl, optionally substituted alkenyl, optionally substituted alkynyl, optionally substituted aryl, or absent;
each Y⁴ is, independently, H, hydroxy, thiol, optionally substituted alkyl, optionally substituted alkenyl, optionally substituted alkynyl, optionally substituted alkoxy, optionally substituted alkenyloxy, optionally substituted alkynyloxy, optionally substituted thioalkoxy, optionally substituted alkoxyalkoxy, or optionally substituted amino;
each Y⁵ is, independently, O, S, optionally substituted alkylene, or optionally substituted heteroalkylene;
n is an integer from 1 to 100,000; and
B is a nucleobase, wherein the combination of B and R^{1′}, the combination of B and R^{2′}, the combination of B and R^{1″}, or the combination of B and R^{2″} can, taken together with the carbons to which they are attached, optionally form a bicyclic group or wherein the combination of B, R^{1″}, and R³ or the combination of B, R^{2″}, and R³ can optionally form a tricyclic or tetracyclic group.

Aspect 8. The composition of any one of aspects 1-7, wherein the composition comprises a lipid, and wherein the lipid is selected from DLin-DMA, DLin-K-DMA, DLin-KC2-DMA, 98N12-5, C12-200, DLin-MC3-DMA, DODMA, DSDMA, DLenDMA, reLNPs, PLGA, a PEGylated lipid, and mixtures of two or more of the foregoing.

Aspect 9. The composition of any one of aspects 1-7, wherein the composition comprises a polymer.

Aspect 10. The composition of aspect 9, herein the polymer is selected from polyethylene glycol, polyglycolide, polyvinyl alcohol, polyvinyl pyrrolidone, polylactide, poly(lactide-co-glycolide) (PLGA), polycaprolactone, polysorbate, polyethylene oxide, polypropylene oxide, poly(ethylene oxide-co-propylene oxide), poloxamer, poloxamine, poly(oxyethylated) glycerol, poly(oxyethylated) sorbitol, poly(oxyethylated) glucose, polyethyleneimine, polyamidoamine (PAMAM) dendrimer, and block copolymer poly(ethylene glycol)-block-poly(lactic-co-glycolic acid) (PEG-b-PLGA).

Aspect 11. The composition of any one of aspects 1-10, wherein the HCV E1 polypeptide is a soluble HCV E1 polypeptide.

Aspect 12. The composition of any one of aspects 1-10, wherein the HCV E2 polypeptide is a soluble HCV E2 polypeptide.

Aspect 13. The composition of any one of aspects 1-12, wherein the nucleotide sequence encodes an HCV E1 polypeptide and an HCV E2 polypeptide.

Aspect 14. The composition of aspect 13, wherein:
a) the HCV E1 polypeptide is a soluble HCV E1 polypeptide;
b) the HCV E2 polypeptide is a soluble HCV E2 polypeptide; or
c) the HCV E1 polypeptide is a soluble HCV E1 polypeptide and the HCV E2 polypeptide is a soluble HCV E2 polypeptide.

Aspect 15. The composition of any one of aspects 1-14, wherein the polypeptide-encoding region comprises a nucleotide sequence encoding an HCV Core polypeptide.

Aspect 16. The composition of aspect 15, wherein the polypeptide-encoding region comprises a nucleotide sequence encoding an HCV Core-E1-E2 polyprotein.

Aspect 17. The composition of any one of aspects 1-16, wherein the polypeptide-encoding region comprises: a) one or more nucleotide sequences encoding one or more heterologous polypeptides comprising a T-cell epitope present in an HCV protein other than E1 and E2; or b) a nucleotide sequence encoding a fusion polypeptide comprising two or more heterologous polypeptides comprising a T-cell epitope present in an HCV protein other than E1 and E2.

Aspect 18. The composition of aspect 17, wherein the RNA comprises: a) an internal ribosome entry site (IRES) between i) the one or more nucleotide sequences encoding the HCV E1 and/or E2 polypeptides; and ii) the one or more nucleotide sequences encoding the one or more heterologous polypeptides or the nucleotide sequence encoding the fusion polypeptide; or b) a nucleotide sequence encoding a self-cleaving peptide between i) the one or more nucleotide sequences encoding the HCV E1 and/or E2 polypeptides; and ii) the one or more nucleotide sequences encoding the one or more heterologous polypeptides or the nucleotide sequence encoding the fusion polypeptide.

Aspect 19. The composition of aspect 17 or aspect 18, wherein the one or more heterologous polypeptides is selected from:
i) a TP35-NS3 T-cell epitope polypeptide comprising an amino acid sequence having at least 80% amino acid sequence identity to: KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₅ is K or R, wherein the TP35-NS3 T-cell epitope polypeptide has a length of from 28 amino acids to 35 amino acids;
ii) a TP50C T-cell epitope polypeptide comprising an amino acid sequence having at least 80% amino acid sequence identity to: GVYX₁LPRRGPRLGVRX₂TRKX₃SERSQPRGRRQX₄IPKX₅X₆X₇X₈X₉GXIoX₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of from 40 amino acids to 50 amino acids;
iii) a TP23 T-cell epitope polypeptide comprising an amino acid sequence having at least 80% amino acid sequence identity to: DVVVX₁X₂TDALMTGX₃TGDFDSVID (SEQ ID NO:257), wherein X₁ is V or C; X₂ is A or S; and X₃ is F or Y, wherein the TP23 T-cell epitope polypeptide has a length of from 18 amino acids to 23 amino acids;
iv) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids;
v) a TP35-NS4 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀ (SEQ ID NO:288), wherein X₁ is I or V; X₂ is L or I; X₃ is R or K; X₄ is V, I, or T; X₅ is P, Q, or T; X₆ is G, A, or S; X₇ is A or V; X₅ is V or T; X₉ is S or A; and X₁₀ is V or I, wherein the TP35-NS4 T-cell epitope polypeptide has a length of from 28 amino acids to 35 amino acids;
vi) a TP42 T-cell epitope polypeptide comprising an amino acid sequence having at least 80% amino acid sequence identity to: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:314), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S, wherein n is an integer from 2 to 10, and wherein the TP42 T-cell epitope polypeptide has a length of from 34 amino acids to 52 amino acids;
vii) a TP45 T-cell epitope polypeptide comprising an amino acid sequence having at least 80% amino acid sequence identity to: X₁X₂AVAX₃YRGX₄DVX₅X₆IPX₇X₈GDVVVX₉X₁₀TDALMTGX₁₁TGDFDSVIDX₁₂X₁₃X₁₄(K)n (SEQ ID NO:332), wherein X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₂ is F or Y; X₁₂ is C or K; X₁₃ is N or K; and X₁₄ is V or K, wherein n is an integer from 2 to 10, and wherein the TP45 T-cell epitope polypeptide has a length of from 36 amino acids to 55 amino acids; and
viii) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acids;
ix) a TP33 T-cell epitope polypeptide comprising an amino acid sequence having at least 80% amino acid sequence identity to: HSKKKCDELAX₁X₂LX₃X₄X₅GX₆NAVAYYRGLDVSX₇IP (SEQ ID NO:366), where X₁ is A or S; X₂ is K or A; X₃ is V, S, R, or T; X₄ is A or G; X₅ is L or M; X₆ is I, L, or V; and X₇ is V or I; wherein the TP33 T-cell epitope polypeptide has a length of from 30 amino acids to 36 amino acids; and
x) a TP42-2 T-cell epitope polypeptide comprising an amino acid sequence having at least 80% amino acid sequence identity to: KGGRHLIFCHSKKKCDELAX₁X₂LX₃X₄X₅GX₆NAVAYYRGLDVSX₇IP (SEQ ID NO:371), where X₁ is A or S; X₂ is K or A; X₃ is V, S, R, or T; X₄ is A or G; X₅ is L or M; X₆ is I, L, or V; and X₇ is V or I; wherein the TP42-2 T-cell epitope polypeptide has a length of from 38 amino acids to 46 amino acids;

Aspect 20. The composition of any one of aspects 17-19, wherein the polypeptide-encoding region comprises one or more nucleotide sequences encoding two or more heterologous polypeptides comprising a T-cell epitope present in an HCV protein other than E1 and E2.

Aspect 21. The composition of aspect 20, wherein the mRNA comprises: a) a first internal ribosome entry site (IRES) between i) the one or more nucleotide sequences encoding the HCV E1 and/or E2 polypeptides; and ii) the nucleotide sequence encoding the first of the two or more heterologous polypeptides; and b) a second IRES between i) the nucleotide sequence encoding the first of the two or more heterologous polypeptides; and ii) the nucleotide sequence encoding the second of the two or more heterologous polypeptides, wherein the first IRES and the second IRES are the same or different.

Aspect 22. The composition of aspect 21, wherein the mRNA comprises an IRES between any two consecutive nucleotide sequences encoding heterologous polypeptides.

Aspect 23. The composition of aspect 17 or aspect 18, wherein fusion polypeptide comprises from 2 to 10 polypeptides selected from the group consisting of:
i) a TP35-NS3 T-cell epitope polypeptide comprising an amino acid sequence having at least 80% amino acid sequence identity to: KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids; and
ii) a TP50C T-cell epitope polypeptide comprising an amino acid sequence having at least 80% amino acid sequence identity to: GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; Xⱼ is R or K; X₁₁ is S, T, H, or A; X₁₂ is A or G; and X₁₃ is Q or K, wherein the TP50C T-cell epitope polypeptide has a length of from 40 amino acids to 50 amino acids;
iii) a TP23 T-cell epitope polypeptide comprising an amino acid sequence having at least 80% amino acid sequence identity to: DVVVX₁X₂TDALMTGX₃TGDFDSVID (SEQ ID NO:257), wherein X₁ is V or C; X₂ is A or S; and X₃ is F or Y, wherein the TP23 T-cell epitope polypeptide has a length of from 18 amino acids to 23 amino acids;
iv) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids;
v) a TP35-NS4 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀ (SEQ ID NO:288), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; and X₁₀ is V or I, wherein the TP35-NS4 T-cell epitope polypeptide has a length of from 28 amino acids to 35 amino acids;
vi) a TP42 T-cell epitope polypeptide comprising an amino acid sequence having at least 80% amino acid sequence identity to: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:314), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S, wherein n is an integer from 2 to 10, and wherein the TP42 T-cell epitope polypeptide has a length of from 34 amino acids to 52 amino acids;
vii) a TP45 T-cell epitope polypeptide comprising an amino acid sequence having at least 80% amino acid sequence identity to: X₁X₂AVAX₃YRGX₄DVX₅X₆IPX₇X₈GDVVVX₉X₁₀TDALMTGX₁₁TGDFDSVIDX₁₂X₁₃X₁₄(K)n (SEQ ID NO:332), wherein X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₂ is F or Y; X₁₂ is C or K; X₁₃ is N or K; and X₁₄ is V or K, wherein n is an integer from 2 to 10, and wherein the TP45 T-cell epitope polypeptide has a length of from 36 amino acids to 55 amino acids; and
viii) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acids;
ix) a TP33 T-cell epitope polypeptide comprising an amino acid sequence having at least 80% amino acid sequence identity to: HSKKKCDELAX₁X₂LX₃X₄X₅GX₆NAVAYYRGLDVSX₇IP (SEQ ID NO:366), where X₁ is A or S; X₂ is K or A; X₃ is V, S, R, or T; X₄ is A or G; X₅ is L or M; X₆ is I, L, or V; and X₇ is V or I; wherein the TP33 T-cell epitope polypeptide has a length of from 30 amino acids to 36 amino acids; and
x) a TP42-2 T-cell epitope polypeptide comprising an amino acid sequence having at least 80% amino acid sequence identity to: KGGRHLIFCHSKKKCDELAX₁X₂LX₃X₄X₅GX₆NAVAYYRGLDVSX₇IP (SEQ ID NO:371), where X₁ is A or S; X₂ is K or A; X₃ is V, S, R, or T; X₄ is A or G; X₅ is L or M; X₆ is I, L, or V; and X₇ is V or I; wherein the TP42-2 T-cell epitope polypeptide has a length of from 38 amino acids to 46 amino acids.

Aspect 24. The composition of any one of aspects 1-23, wherein the HCV E1 and/or E2 polypeptide is from HCV genotype 1, optionally wherein the HCV E1 and/or E2 polypeptide is from HCV genotype 1a.

Aspect 25. The composition of any one of aspects 1-23, wherein the HCV E1 and/or E2 polypeptide is from HCV genotype 2, optionally wherein the HCV E1 and/or E2 polypeptide is from HCV genotype 2a.

Aspect 26. The composition of any one of aspects 1-23, wherein the HCV E1 and/or E2 polypeptide is from HCV genotype 3, optionally wherein the HCV E1 and/or E2 polypeptide is from HCV genotype 3a.

Aspect 27. The composition of any one of aspects 1-23, wherein the HCV E1 and/or E2 polypeptide is/are from:
a) HCV genotype 1, 2, and 3;
b) HCV genotype 1a, 2, and 3;
c) HCV genotype 1b, 2, and 3; or
d) HCV genotype 1a, 1b, 2, and 3.

Aspect 28. The composition of any one of aspects 24-27, wherein the polypeptide-encoding region comprises a nucleotide sequence encoding an HCV Core polypeptide, wherein the HCV Core polypeptide is of genotype 1, genotype 2, or genotype 3.

Aspect 29. The composition of any one of aspects 17-28, wherein the one or more RNA molecules comprises a first RNA molecule, a second RNA molecule, and a third RNA molecule, wherein:
a) the polypeptide-encoding region of the first RNA molecule comprises: i) a nucleotide sequence encoding HCV E1 and/or an HCV E2 polypeptide of genotype 1; ii) a first IRES; and iii) one or more nucleotide sequences encoding the one or more heterologous polypeptides or the fusion polypeptide;
b) the polypeptide-encoding region of the second RNA molecule comprises: i) a nucleotide sequence encoding HCV E1 and/or an HCV E2 polypeptide of genotype 2; ii) a second IRES; and iii) one or more nucleotide sequences encoding the one or more heterologous polypeptides or the fusion polypeptide; and
c) the polypeptide-encoding region of the third RNA molecule comprises: i) a nucleotide sequence encoding HCV E1 and/or an HCV E2 polypeptide of genotype 3; ii) a third IRES; and iii) one or more nucleotide sequences encoding the one or more heterologous polypeptides or the fusion polypeptide; wherein the first IRES, the second IRES, and the third IRES are the same or different.

Aspect 30. The composition of aspect 29, wherein:
a) the HCV E1 polypeptide encoded by the first RNA molecule is a soluble HCV E1 polypeptide and/or the HCV E2 polypeptide encoded by the first RNA molecule is a soluble HCV E2 polypeptide; and/or
b) the HCV E1 polypeptide encoded by the second RNA molecule is a soluble HCV E1 polypeptide and/or the HCV E2 polypeptide encoded by the second RNA molecule is a soluble HCV E2 polypeptide; and/or
c) the HCV E1 polypeptide encoded by the third RNA molecule is a soluble HCV E1 polypeptide and/or the HCV E2 polypeptide encoded by the third RNA molecule is a soluble HCV E2 polypeptide.

Aspect 31. The composition of aspect 29 or aspect 30, wherein the polypeptide-encoding region of one or more of the first RNA molecule, the second RNA molecule, and the third RNA molecule comprises a nucleotide sequence encoding an HCV Core polypeptide.

Aspect 32. The composition of aspect 29 or aspect 30, wherein: a) the polypeptide-encoding region of the first RNA molecule comprises: i) a nucleotide sequence encoding HCV E1 and HCV E2 polypeptides of genotype 1; ii) a first IRES; and iii) a nucleotide sequence encoding the fusion polypeptide; b) the polypeptide-encoding region of the second RNA molecule comprises: i) a nucleotide sequence encoding HCV E1 and HCV E2 polypeptides of genotype 2; ii) a second IRES; and iii) a nucleotide sequence encoding the fusion polypeptide; and c) the polypeptide-encoding region of the third RNA molecule comprises: i) a nucleotide sequence encoding HCV E1 and HCV E2 polypeptides of genotype 3; ii) a third IRES; and iii) a nucleotide sequence encoding the fusion polypeptide; wherein the first IRES, the second IRES, and the third IRES are the same or different.

Aspect 33. The composition of aspect 32, wherein: a) the HCV E1 polypeptide of genotype 1 comprises an amino acid sequence having at least 95% amino acid sequence identity to an H77 E1 polypeptide, and the HCV E2 polypeptide of genotype 1 comprises an amino acid sequence having at least 95% amino acid sequence identity to an H77 E2 polypeptide; b) the HCV E1 polypeptide of genotype 2 comprises an amino acid sequence having at least 95% amino acid sequence identity to a J6 E1 polypeptide, and the HCV E2 polypeptide of genotype 2 comprises an amino acid sequence having at least 95% amino acid sequence identity to a J6 E2 polypeptide; and c) the HCV E1 polypeptide of genotype 3 comprises an amino acid sequence having at least 95% amino acid sequence identity to an S52 E1 polypeptide, and the HCV E2 polypeptide of genotype 3 comprises an amino acid sequence having at least 95% amino acid sequence identity to an S52 E2 polypeptide.

Aspect 34. The composition of aspect 32 or aspect 33, wherein the polypeptide-encoding region of one or more of the first RNA molecule, the second RNA molecule, and the third RNA molecule comprises a nucleotide sequence encoding an HCV Core polypeptide.

Aspect 35. The composition of aspect 34, wherein: a) the polypeptide-encoding region of the first RNA molecule comprises: i) a nucleotide sequence encoding an HCV Core-E1-E2 polypeptide of genotype 1; ii) a first IRES; and iii) a nucleotide sequence encoding the fusion polypeptide; b) the polypeptide-encoding region of the second RNA molecule comprises: i) a nucleotide sequence encoding an HCV Core-E1-E2 polypeptide of genotype 2; ii) a second IRES; and iii) a nucleotide sequence encoding the fusion polypeptide; and c) the polypeptide-encoding region of the third RNA molecule comprises: i) a nucleotide sequence encoding an HCV Core-E1-E2 polypeptide of genotype 3; ii) a third IRES; and iii) a nucleotide sequence encoding the fusion polypeptide; wherein the first IRES, the second IRES, and the third IRES are the same or different.

Aspect 36. The composition of aspect 35, wherein: a) the HCV E1 polypeptide of genotype 1 comprises an amino acid sequence having at least 95% amino acid sequence identity to an H77 E1 polypeptide, and the HCV E2 polypeptide of genotype 1 comprises an amino acid sequence having at least 95% amino acid sequence identity to an H77 E2 polypeptide; b) the HCV E1 polypeptide of genotype 2 comprises an amino acid sequence having at least 95% amino acid sequence identity to a J6 E1 polypeptide, and the HCV E2 polypeptide of genotype 2 comprises an amino acid sequence having at least 95% amino acid sequence identity to a J6 E2 polypeptide; and c) the HCV E1 polypeptide of genotype 3 comprises an amino acid sequence having at least 95% amino acid sequence identity to an S52 E1 polypeptide, and the HCV E2 polypeptide of genotype 3 comprises an amino acid sequence having at least 95% amino acid sequence identity to an S52 E2 polypeptide.

Aspect 37. The composition of any one of aspects 17 to 28, wherein: a) the polypeptide-encoding region of the first RNA molecule comprises a nucleotide sequence encoding a fusion polypeptide comprising: i) HCV E1 and HCV2 E2 polypeptides of genotype 1; and ii) the fusion polypeptide comprising two or more heterologous polypeptides comprising a T-cell epitope present in an HCV protein other than E1 and E2; b) the polypeptide-encoding region of the second RNA molecule comprises a nucleotide sequence encoding a fusion polypeptide comprising: i) HCV E1 and HCV2 E2 polypeptides of genotype 2; and ii) the fusion polypeptide comprising two or more heterologous polypeptides comprising a T-cell epitope present in an HCV protein other than E1 and E2; and c) the polypeptide-encoding region of the third RNA molecule comprises a nucleotide sequence encoding a fusion polypeptide comprising: i) HCV E1 and HCV2 E2 polypeptides of genotype 3; and ii) the fusion polypeptide comprising two or more heterologous polypeptides comprising a T-cell epitope present in an HCV protein other than E1 and E2.

Aspect 38. The composition of aspect 37, wherein: a) the HCV E1 polypeptide of genotype 1 comprises an amino acid sequence having at least 95% amino acid sequence identity to an H77 E1 polypeptide, and the HCV E2 polypeptide of genotype 1 comprises an amino acid sequence having at least 95% amino acid sequence identity to an H77 E2 polypeptide; b) the HCV E1 polypeptide of genotype 2 comprises an amino acid sequence having at least 95% amino acid sequence identity to a J6 E1 polypeptide, and the HCV E2 polypeptide of genotype 2 comprises an amino acid sequence having at least 95% amino acid sequence identity to a J6 E2 polypeptide; and c) the HCV E1 polypeptide of genotype 3 comprises an amino acid sequence having at least 95% amino acid sequence identity to an S52 E1 polypeptide, and the HCV E2 polypeptide of genotype 3 comprises an amino acid sequence having at least 95% amino acid sequence identity to an S52 E2 polypeptide.

Aspect 39. The composition of aspect 37, wherein: a) the HCV E1 polypeptide encoded by the first RNA molecule is a soluble HCV E1 polypeptide and/or the HCV E2 polypeptide encoded by the first RNA molecule is a soluble HCV E2 polypeptide; and/or b) the HCV E1 polypeptide encoded by the second RNA molecule is a soluble HCV E1 polypeptide and/or the HCV E2 polypeptide encoded by the second RNA molecule is a soluble HCV E2 polypeptide; and/or c) the HCV E1 polypeptide encoded by the third RNA molecule is a soluble HCV E1 polypeptide and/or the HCV E2 polypeptide encoded by the third RNA molecule is a soluble HCV E2 polypeptide.

Aspect 40. The composition of aspect 39, wherein: a) the soluble HCV E1 polypeptide of genotype 1 comprises an amino acid sequence having at least 95% amino acid sequence identity to a soluble H77 E1 polypeptide, and the soluble HCV E2 polypeptide of genotype 1 comprises an amino acid sequence having at least 95% amino acid sequence identity to a soluble H77 E2 polypeptide; b) the soluble HCV E1 polypeptide of genotype 2 comprises an amino acid sequence having at least 95% amino acid sequence identity to a soluble J6 E1 polypeptide, and the soluble HCV E2 polypeptide of genotype 2 comprises an amino acid sequence having at least 95% amino acid sequence identity to a soluble J6 E2 polypeptide; and c) the soluble HCV E1 polypeptide of genotype 3 comprises an amino acid sequence having at least 95% amino acid sequence identity to a soluble S52 E1 polypeptide, and the soluble HCV E2 polypeptide of genotype 3 comprises an amino acid sequence having at least 95% amino acid sequence identity to a soluble S52 E2 polypeptide.

Aspect 41. The composition of any one of aspects 37-40, wherein the polypeptide-encoding region of one or more of the first RNA molecule, the second RNA molecule, and the third RNA molecule comprises a nucleotide sequence encoding an HCV Core polypeptide.

Aspect 42. The composition of aspect 41, wherein: a) the polypeptide-encoding region of the first RNA molecule comprises a nucleotide sequence encoding a fusion polypeptide comprising: i) an HCV Core-E1-E2 polyprotein of genotype 1; and ii) the fusion polypeptide comprising two or more heterologous polypeptides comprising a T-cell epitope present in an HCV protein other than E1 and E2; b) the polypeptide-encoding region of the first RNA molecule comprises a nucleotide sequence encoding a fusion polypeptide comprising: i) an HCV Core-E1-E2 polyprotein of genotype 2; and ii) the fusion polypeptide comprising two or more heterologous polypeptides comprising a T-cell epitope present in an HCV protein other than E1 and E2; and c) the polypeptide-encoding region of the first RNA molecule comprises a nucleotide sequence encoding a fusion polypeptide comprising: i) an HCV Core-E1-E2 polyprotein of genotype 3; and ii) the fusion polypeptide comprising two or more heterologous polypeptides comprising a T-cell epitope present in an HCV protein other than E1 and E2.

Aspect 43. The composition of aspect 42, wherein: a) the HCV Core-E1-E2 polyprotein of genotype 1 comprises an amino acid sequence having at least 95% amino acid sequence identity to an H77 Core-E1-E2 polyprotein; b) the HCV Core-E1-E2 polyprotein of genotype 2 comprises an amino acid sequence having at least 95% amino acid sequence identity to a J6 Core-E1-E2 polyprotein; and c) the HCV Core-E1-E2 polyprotein of genotype 3 comprises an amino acid sequence having at least 95% amino acid sequence identity to an S52 Core-E1-E2 polyprotein.

Aspect 44. The composition of any one of aspects 23-43, wherein the fusion polypeptide comprising two or more heterologous polypeptides comprising a T-cell epitope present in an HCV protein other than E1 and E2 comprises:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; Xⱼ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 46. The composition of any one of aspects 23-45, wherein the fusion polypeptide comprising two or more heterologous polypeptides comprising a T-cell epitope present in an HCV protein other than E1 and E2 comprises, in order from N-terminus to C-terminus:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 47. The composition of any one of aspects 17-28, wherein the one or more RNA molecules comprises a first RNA molecule and a second RNA molecule, wherein: a) the polypeptide-encoding region of the first RNA molecule comprises: i) a nucleotide sequence encoding HCV E1 and/or an HCV E2 polypeptide of genotype 1; ii) a first IRES; and iii) one or more nucleotide sequences encoding the one or more heterologous polypeptides or the fusion polypeptide; and b) the polypeptide-encoding region of the second RNA molecule comprises: i) a nucleotide sequence encoding HCV E1 and/or an HCV E2 polypeptide of genotype 2; ii) a second IRES; and iii) one or more nucleotide sequences encoding the one or more heterologous polypeptides or the fusion polypeptide; wherein the first IRES and the second IRES are the same or different.

Aspect 48. The composition of aspect 47, wherein: a) the HCV E1 polypeptide encoded by the first RNA molecule is a soluble HCV E1 polypeptide and/or the HCV E2 polypeptide encoded by the first RNA molecule is a soluble HCV E2 polypeptide; and/or b) the HCV E1 polypeptide encoded by the second RNA molecule is a soluble HCV E1 polypeptide and/or the HCV E2 polypeptide encoded by the second RNA molecule is a soluble HCV E2 polypeptide.

Aspect 49. The composition of aspect 47 or 48, wherein the polypeptide-encoding region of one or both of the first RNA molecule and the second RNA molecule comprises a nucleotide sequence encoding an HCV Core polypeptide.

Aspect 50. The composition of aspect 47 or 48, wherein: a1) the polypeptide-encoding region of the first RNA molecule comprises: i) a nucleotide sequence encoding HCV E1 and HCV E2 polypeptides of genotype 1; ii) a first IRES; and iii) a nucleotide sequence encoding the fusion polypeptide; and b1) the polypeptide-encoding region of the second RNA molecule comprises: i) a nucleotide sequence encoding HCV E1 and HCV E2 polypeptides of genotype 3; ii) a second IRES; and iii) a nucleotide sequence encoding the fusion polypeptide; or a2) the polypeptide-encoding region of the first RNA molecule comprises: i) a nucleotide sequence encoding HCV E1 and HCV E2 polypeptides of genotype 1; ii) a first IRES; and iii) a nucleotide sequence encoding the fusion polypeptide; and b2) the polypeptide-encoding region of the second RNA molecule comprises: i) a nucleotide sequence encoding HCV E1 and HCV E2 polypeptides of genotype 2; ii) a second IRES; and iii) a nucleotide sequence encoding the fusion polypeptide; or a3) the polypeptide-encoding region of the first RNA molecule comprises: i) a nucleotide sequence encoding HCV E1 and HCV E2 polypeptides of genotype 2; ii) a first IRES; and iii) a nucleotide sequence encoding the fusion polypeptide; and b3) the polypeptide-encoding region of the second RNA molecule comprises: i) a nucleotide sequence encoding HCV E1 and HCV E2 polypeptides of genotype 3; ii) a second IRES; and iii) a nucleotide sequence encoding the fusion polypeptide; wherein the first IRES and the second IRES are the same or different.

Aspect 51. The composition of aspect 50, wherein: a) the HCV E1 polypeptide of genotype 1 comprises an amino acid sequence having at least 95% amino acid sequence identity to an H77 E1 polypeptide, and the HCV E2 polypeptide of genotype 1 comprises an amino acid sequence having at least 95% amino acid sequence identity to an H77 E2 polypeptide; b) the HCV E1 polypeptide of genotype 2 comprises an amino acid sequence having at least 95% amino acid sequence identity to a J6 E1 polypeptide, and the HCV E2 polypeptide of genotype 2 comprises an amino acid sequence having at least 95% amino acid sequence identity to a J6 E2 polypeptide; and c) the HCV E1 polypeptide of genotype 3 comprises an amino acid sequence having at least 95% amino acid sequence identity to an S52 E1 polypeptide, and the HCV E2 polypeptide of genotype 3 comprises an amino acid sequence having at least 95% amino acid sequence identity to an S52 E2 polypeptide.

Aspect 52. The composition of aspect 50 or aspect 51, wherein the polypeptide-encoding region of one or both of the first RNA molecule and the second RNA molecule comprises a nucleotide sequence encoding an HCV Core polypeptide.

Aspect 53. The composition of aspect 52, wherein: a1) the polypeptide-encoding region of the first RNA molecule comprises: i) a nucleotide sequence encoding an HCV Core-E1-E2 polyprotein of genotype 1; ii) a first IRES; and iii) a nucleotide sequence encoding the fusion polypeptide; b1) the polypeptide-encoding region of the second RNA molecule comprises: i) a nucleotide sequence encoding an HCV Core-E1-E2 polyprotein of genotype 3; ii) a second IRES; and iii) a nucleotide sequence encoding the fusion polypeptide; or a2) the polypeptide-encoding region of the first RNA molecule comprises: i) a nucleotide sequence encoding an HCV Core-E1-E2 polyprotein of genotype 1; ii) a first IRES; and iii) a nucleotide sequence encoding the fusion polypeptide; b2) the polypeptide-encoding region of the second RNA molecule comprises: i) a nucleotide sequence encoding an HCV Core-E1-E2 polyprotein of genotype 2; ii) a second IRES; and iii) a nucleotide sequence encoding the fusion polypeptide; or a3) the polypeptide-encoding region of the first RNA molecule comprises: i) a nucleotide sequence encoding an HCV Core-E1-E2 polyprotein of genotype 2; ii) a first IRES; and iii) a nucleotide sequence encoding the fusion polypeptide; b3) the polypeptide-encoding region of the second RNA molecule comprises: i) a nucleotide sequence encoding an HCV Core-E1-E2 polyprotein of genotype 3; ii) a second IRES; and iii) a nucleotide sequence encoding the fusion polypeptide; wherein the first IRES and the second IRES are the same or different.

Aspect 54. The composition of aspect 53, wherein: a) the HCV Core-E1-E2 polyprotein of genotype 1 comprises an amino acid sequence having at least 95% amino acid sequence identity to an H77 HCV Core-E1-E2 polyprotein; b) the HCV Core-E1-E2 polyprotein of genotype 2 comprises an amino acid sequence having at least 95% amino acid sequence identity to a J6 HCV Core-E1-E2 polyprotein; and c) the HCV Core-E1-E2 polyprotein of genotype 3 comprises an amino acid sequence having at least 95% amino acid sequence identity to an S52 HCV Core-E1-E2 polyprotein.

Aspect 55. The composition of any one of aspects 17 to 28, wherein the composition comprises a first RNA molecule and a second RNA molecule, and wherein: a1) the polypeptide-encoding region of the first RNA molecule comprises a nucleotide sequence encoding a fusion polypeptide comprising: i) HCV E1 and HCV2 E2 polypeptides of genotype 1; and ii) the fusion polypeptide comprising two or more heterologous polypeptides comprising a T-cell epitope present in an HCV protein other than E1 and E2; b1) the polypeptide-encoding region of the second RNA molecule comprises a nucleotide sequence encoding a fusion polypeptide comprising: i) HCV E1 and HCV2 E2 polypeptides of genotype 3; and ii) the fusion polypeptide comprising two or more heterologous polypeptides comprising a T-cell epitope present in an HCV protein other than E1 and E2; or a2) the polypeptide-encoding region of the first RNA molecule comprises a nucleotide sequence encoding a fusion polypeptide comprising: i) HCV E1 and HCV2 E2 polypeptides of genotype 1; and ii) the fusion polypeptide comprising two or more heterologous polypeptides comprising a T-cell epitope present in an HCV protein other than E1 and E2; b2) the polypeptide-encoding region of the second RNA molecule comprises a nucleotide sequence encoding a fusion polypeptide comprising: i) HCV E1 and HCV2 E2 polypeptides of genotype 2; and ii) the fusion polypeptide comprising two or more heterologous polypeptides comprising a T-cell epitope present in an HCV protein other than E1 and E2; or a3) the polypeptide-encoding region of the first RNA molecule comprises a nucleotide sequence encoding a fusion polypeptide comprising: i) HCV E1 and HCV2 E2 polypeptides of genotype 2; and ii) the fusion polypeptide comprising two or more heterologous polypeptides comprising a T-cell epitope present in an HCV protein other than E1 and E2; b3) the polypeptide-encoding region of the second RNA molecule comprises a nucleotide sequence encoding a fusion polypeptide comprising: i) HCV E1 and HCV2 E2 polypeptides of genotype 3; and ii) the fusion polypeptide comprising two or more heterologous polypeptides comprising a T-cell epitope present in an HCV protein other than E1 and E2.

Aspect 56. The composition of aspect 55, wherein: a) the HCV E1 polypeptide of genotype 1 comprises an amino acid sequence having at least 95% amino acid sequence identity to an H77 E1 polypeptide, and the HCV E2 polypeptide of genotype 1 comprises an amino acid sequence having at least 95% amino acid sequence identity to an H77 E2 polypeptide; b) the HCV E1 polypeptide of genotype 2 comprises an amino acid sequence having at least 95% amino acid sequence identity to a J6 E1 polypeptide, and the HCV E2 polypeptide of genotype 2 comprises an amino acid sequence having at least 95% amino acid sequence identity to a J6 E2 polypeptide; and c) the HCV E1 polypeptide of genotype 3 comprises an amino acid sequence having at least 95% amino acid sequence identity to an S52 E1 polypeptide, and the HCV E2 polypeptide of genotype 3 comprises an amino acid sequence having at least 95% amino acid sequence identity to an S52 E2 polypeptide.

Aspect 57. The composition of aspect 55, wherein: a) the HCV E1 polypeptide encoded by the first RNA molecule is a soluble HCV E1 polypeptide and/or the HCV E2 polypeptide encoded by the first RNA molecule is a soluble HCV E2 polypeptide; and/or b) the HCV E1 polypeptide encoded by the second RNA molecule is a soluble HCV E1 polypeptide and/or the HCV E2 polypeptide encoded by the second RNA molecule is a soluble HCV E2 polypeptide.

Aspect 58. The composition of aspect 57, wherein: a) the soluble HCV E1 polypeptide of genotype 1 comprises an amino acid sequence having at least 95% amino acid sequence identity to a soluble H77 E1 polypeptide, and the soluble HCV E2 polypeptide of genotype 1 comprises an amino acid sequence having at least 95% amino acid sequence identity to a soluble H77 E2 polypeptide; and/or b) the soluble HCV E1 polypeptide of genotype 2 comprises an amino acid sequence having at least 95% amino acid sequence identity to a soluble J6 E1 polypeptide, and the soluble HCV E2 polypeptide of genotype 2 comprises an amino acid sequence having at least 95% amino acid sequence identity to a soluble J6 E2 polypeptide; and/or c) the soluble HCV E1 polypeptide of genotype 3 comprises an amino acid sequence having at least 95% amino acid sequence identity to a soluble S52 E1 polypeptide, and the soluble HCV E2 polypeptide of genotype 3 comprises an amino acid sequence having at least 95% amino acid sequence identity to a soluble S52 E2 polypeptide.

Aspect 59. The composition of any one of aspects 55-58, wherein the polypeptide-encoding region of one or both of the first RNA molecule and the second RNA molecule comprises a nucleotide sequence encoding an HCV Core polypeptide.

Aspect 60. The composition of aspect 59, wherein: a1) the polypeptide-encoding region of the first RNA molecule comprises a nucleotide sequence encoding a fusion polypeptide comprising: i) an HCV Core-E1-E2 polyprotein of genotype 1; and ii) the fusion polypeptide comprising two or more heterologous polypeptides comprising a T-cell epitope present in an HCV protein other than E1 and E2; b1) the polypeptide-encoding region of the first RNA molecule comprises a nucleotide sequence encoding a fusion polypeptide comprising: i) an HCV Core-E1-E2 polyprotein of genotype 3; and ii) the fusion polypeptide comprising two or more heterologous polypeptides comprising a T-cell epitope present in an HCV protein other than E1 and E2; or a2) the polypeptide-encoding region of the first RNA molecule comprises a nucleotide sequence encoding a fusion polypeptide comprising: i) an HCV Core-E1-E2 polyprotein of genotype 1; and ii) the fusion polypeptide comprising two or more heterologous polypeptides comprising a T-cell epitope present in an HCV protein other than E1 and E2; b2) the polypeptide-encoding region of the first RNA molecule comprises a nucleotide sequence encoding a fusion polypeptide comprising: i) an HCV Core-E1-E2 polyprotein of genotype 2; and ii) the fusion polypeptide comprising two or more heterologous polypeptides comprising a T-cell epitope present in an HCV protein other than E1 and E2; or a3) the polypeptide-encoding region of the first RNA molecule comprises a nucleotide sequence encoding a fusion polypeptide comprising: i) an HCV Core-E1-E2 polyprotein of genotype 2; and ii) the fusion polypeptide comprising two or more heterologous polypeptides comprising a T-cell epitope present in an HCV protein other than E1 and E2; b3) the polypeptide-encoding region of the first RNA molecule comprises a nucleotide sequence encoding a fusion polypeptide comprising: i) an HCV Core-E1-E2 polyprotein of genotype 3; and ii) the fusion polypeptide comprising two or more heterologous polypeptides comprising a T-cell epitope present in an HCV protein other than E1 and E2.

Aspect 61. The composition of aspect 60, wherein: a) the HCV Core-E1-E2 polyprotein of genotype 1 comprises an amino acid sequence having at least 95% amino acid sequence identity to an H77 Core-E1-E2 polyprotein; b) the HCV Core-E1-E2 polyprotein of genotype 2 comprises an amino acid sequence having at least 95% amino acid sequence identity to a J6 Core-E1-E2 polyprotein; and c) the HCV Core-E1-E2 polyprotein of genotype 3 comprises an amino acid sequence having at least 95% amino acid sequence identity to an S52 Core-E1-E2 polyprotein.

Aspect 62. The composition of any one of aspects 47-61, wherein the fusion polypeptide comprising two or more heterologous polypeptides comprising a T-cell epitope present in an HCV protein other than E1 and E2 comprises:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 63. The composition of any one of aspects 47-61, wherein the fusion polypeptide comprising two or more heterologous polypeptides comprising a T-cell epitope present in an HCV protein other than E1 and E2 comprises, in order from N-terminus to C-terminus:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 64. The composition of any one of aspects 17-28, wherein the composition comprises a single RNA molecule, wherein: a) the polypeptide-encoding region of the RNA molecule comprises: i) a nucleotide sequence encoding an HCV E1 and/or an HCV E2 polypeptide of genotype 1; ii) a first IRES; and iii) one or more nucleotide sequences encoding the one or more heterologous polypeptides or the fusion polypeptide; or b) the polypeptide-encoding region of the RNA molecule comprises: i) a nucleotide sequence encoding an HCV E1 and/or an HCV E2 polypeptide of genotype 2; ii) a first IRES; and iii) one or more nucleotide sequences encoding the one or more heterologous polypeptides or the fusion polypeptide; or c) the polypeptide-encoding region of the RNA molecule comprises: i) a nucleotide sequence encoding an HCV E1 and/or an HCV E2 polypeptide of genotype 3; ii) a first IRES; and iii) one or more nucleotide sequences encoding the one or more heterologous polypeptides or the fusion polypeptide.

Aspect 65. The composition of aspect 64, wherein: a) the HCV E1 polypeptide of genotype 1 comprises an amino acid sequence having at least 95% amino acid sequence identity to an H77 E1 polypeptide, and the HCV E2 polypeptide of genotype 1 comprises an amino acid sequence having at least 95% amino acid sequence identity to an H77 E2 polypeptide; b) the HCV E1 polypeptide of genotype 2 comprises an amino acid sequence having at least 95% amino acid sequence identity to a J6 E1 polypeptide, and the HCV E2 polypeptide of genotype 2 comprises an amino acid sequence having at least 95% amino acid sequence identity to a J6 E2 polypeptide; and c) the HCV E1 polypeptide of genotype 3 comprises an amino acid sequence having at least 95% amino acid sequence identity to an S52 E1 polypeptide, and the HCV E2 polypeptide of genotype 3 comprises an amino acid sequence having at least 95% amino acid sequence identity to an S52 E2 polypeptide.

Aspect 66. The composition of aspect 64 or aspect 65, wherein the polypeptide-encoding region of the RNA molecule comprises a nucleotide sequence encoding an HCV Core polypeptide.

Aspect 67. The composition of aspect 66, wherein: a) the polypeptide-encoding region of the RNA molecule comprises: i) a nucleotide sequence encoding an HCV Core-E1-E2 polyprotein of genotype 1; ii) a first IRES; and iii) one or more nucleotide sequences encoding the one or more heterologous polypeptides or the fusion polypeptide; or b) the polypeptide-encoding region of the RNA molecule comprises: i) a nucleotide sequence encoding an HCV Core-E1-E2 polyprotein of genotype 2; ii) a first IRES; and iii) one or more nucleotide sequences encoding the one or more heterologous polypeptides or the fusion polypeptide; or c) the polypeptide-encoding region of the RNA molecule comprises: i) a nucleotide sequence encoding an HCV Core-E1-E2 polyprotein of genotype 3; ii) a first IRES; and iii) one or more nucleotide sequences encoding the one or more heterologous polypeptides or the fusion polypeptide.

Aspect 68. The composition of aspect 67, wherein: a) the HCV Core-E1-E2 polyprotein of genotype 1 comprises an amino acid sequence having at least 95% amino acid sequence identity to an H77 HCV Core-E1-E2 polyprotein; b) the HCV Core-E1-E2 polyprotein of genotype 2 comprises an amino acid sequence having at least 95% amino acid sequence identity to a J6 HCV Core-E1-E2 polyprotein; and c) the HCV Core-E1-E2 polyprotein of genotype 3 comprises an amino acid sequence having at least 95% amino acid sequence identity to an S52 HCV Core-E1-E2 polyprotein.

Aspect 69. The composition of any one of aspects 17 to 28, wherein the composition comprises a single RNA molecule, and wherein: a) the polypeptide-encoding region of the RNA molecule comprises a nucleotide sequence encoding a fusion polypeptide comprising: i) HCV E1 and HCV2 E2 polypeptides of genotype 1; and ii) the fusion polypeptide comprising two or more heterologous polypeptides comprising a T-cell epitope present in an HCV protein other than E1 and E2; or b) the polypeptide-encoding region of the RNA molecule comprises a nucleotide sequence encoding a fusion polypeptide comprising: i) HCV E1 and HCV2 E2 polypeptides of genotype 2; and ii) the fusion polypeptide comprising two or more heterologous polypeptides comprising a T-cell epitope present in an HCV protein other than E1 and E2; or c) the polypeptide-encoding region of the RNA molecule comprises a nucleotide sequence encoding a fusion polypeptide comprising: i) HCV E1 and HCV2 E2 polypeptides of genotype 3; and ii) the fusion polypeptide comprising two or more heterologous polypeptides comprising a T-cell epitope present in an HCV protein other than E1 and E2.

Aspect 70. The composition of aspect 69, wherein: a) the HCV E1 polypeptide of genotype 1 comprises an amino acid sequence having at least 95% amino acid sequence identity to an H77 E1 polypeptide, and the HCV E2 polypeptide of genotype 1 comprises an amino acid sequence having at least 95% amino acid sequence identity to an H77 E2 polypeptide; b) the HCV E1 polypeptide of genotype 2 comprises an amino acid sequence having at least 95% amino acid sequence identity to a J6 E1 polypeptide, and the HCV E2 polypeptide of genotype 2 comprises an amino acid sequence having at least 95% amino acid sequence identity to a J6 E2 polypeptide; and c) the HCV E1 polypeptide of genotype 3 comprises an amino acid sequence having at least 95% amino acid sequence identity to an S52 E1 polypeptide, and the HCV E2 polypeptide of genotype 3 comprises an amino acid sequence having at least 95% amino acid sequence identity to an S52 E2 polypeptide.

Aspect 71. The composition of aspect 69, wherein: a) the HCV E1 polypeptide encoded by the RNA molecule is a soluble HCV E1 polypeptide; and/or b) the HCV E2 polypeptide encoded by the RNA molecule is a soluble HCV E2 polypeptide.

Aspect 72. The composition of aspect 71, wherein: a) the soluble HCV E1 polypeptide of genotype 1 comprises an amino acid sequence having at least 95% amino acid sequence identity to a soluble H77 E1 polypeptide, and the soluble HCV E2 polypeptide of genotype 1 comprises an amino acid sequence having at least 95% amino acid sequence identity to a soluble H77 E2 polypeptide; b) the soluble HCV E1 polypeptide of genotype 2 comprises an amino acid sequence having at least 95% amino acid sequence identity to a soluble J6 E1 polypeptide, and the soluble HCV E2 polypeptide of genotype 2 comprises an amino acid sequence having at least 95% amino acid sequence identity to a soluble J6 E2 polypeptide; and c) the soluble HCV E1 polypeptide of genotype 3 comprises an amino acid sequence having at least 95% amino acid sequence identity to a soluble S52 E1 polypeptide, and the soluble HCV E2 polypeptide of genotype 3 comprises an amino acid sequence having at least 95% amino acid sequence identity to a soluble S52 E2 polypeptide.

Aspect 73. The composition of any one of aspects 69-72, wherein the polypeptide-encoding region of the RNA molecule comprises a nucleotide sequence encoding an HCV Core polypeptide.

Aspect 74. The composition of aspect 73, wherein: a) the polypeptide-encoding region of the RNA molecule comprises a nucleotide sequence encoding a fusion polypeptide comprising: i) an HCV Core-E1-E2 polyprotein of genotype 1; and ii) the fusion polypeptide comprising two or more heterologous polypeptides comprising a T-cell epitope present in an HCV protein other than E1 and E2; or b) the polypeptide-encoding region of the RNA molecule comprises a nucleotide sequence encoding a fusion polypeptide comprising: i) an HCV Core-E1-E2 polyprotein of genotype 2; and ii) the fusion polypeptide comprising two or more heterologous polypeptides comprising a T-cell epitope present in an HCV protein other than E1 and E2; or c) the polypeptide-encoding region of the RNA molecule comprises a nucleotide sequence encoding a fusion polypeptide comprising: i) an HCV Core-E1-E2 polyprotein of genotype 3; and ii) the fusion polypeptide comprising two or more heterologous polypeptides comprising a T-cell epitope present in an HCV protein other than E1 and E2.

Aspect 75. The composition of aspect 74, wherein: a) the HCV Core-E1-E2 polyprotein of genotype 1 comprises an amino acid sequence having at least 95% amino acid sequence identity to an H77 Core-E1-E2 polyprotein; b) the HCV Core-E1-E2 polyprotein of genotype 2 comprises an amino acid sequence having at least 95% amino acid sequence identity to a J6 Core-E1-E2 polyprotein; and c) the HCV Core-E1-E2 polyprotein of genotype 3 comprises an amino acid sequence having at least 95% amino acid sequence identity to an S52 Core-E1-E2 polyprotein.

Aspect 76. The composition of any one of aspects 64-75, wherein the fusion polypeptide comprising two or more heterologous polypeptides comprising a T-cell epitope present in an HCV protein other than E1 and E2 comprises:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 77. The composition of any one of aspects 64-75, wherein the fusion polypeptide comprising two or more heterologous polypeptides comprising a T-cell epitope present in an HCV protein other than E1 and E2 comprises, in order from N-terminus to C-terminus:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GXIoX₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 78. The composition of any one of aspects 1-77, wherein the RNA is a self-amplifying RNA comprising a nucleotide sequence encoding an RNA-dependent RNA polymerase (RdRP).

Aspect 79. The composition of aspect 78, wherein the self-amplifying RNA is a cis-replicon.

Aspect 80. The composition of aspect 78 or aspect 79, wherein the self-amplifying RNA is a recombinant Venezuelan equine encephalitis virus, a Sindbis virus, or a recombinant Semliki Forest virus.

Aspect 81. The composition of any one of aspects 1-80, wherein the composition is in the form of a lipid nanoparticle.

Aspect 82. The composition of any one of aspects 1-80, wherein the composition is in the form of a liposome.

Aspect 83. The composition of any one of aspects 1-80, wherein the composition comprises one or more lipids selected from cationic lipids, neutral lipids, anionic lipids, helper lipids, and stealth lipids.

Aspect 84. The composition of any one of aspects 1-80, wherein the composition comprises an HCV Core polypeptide and wherein the one or more RNA molecules are within a virus-like particle comprising the HCV Core polypeptide.

Aspect 85. A method of inducing an immune response to hepatitis C virus (HCV) in an individual, the method comprising administering to the individual an effective amount of a composition of any one of aspects 1-84.

Aspect 86. The method of aspect 85, comprising:
a) administering a first dose of the composition at a first time; and
b) administering a second dose of the composition at a second time.

Aspect 87. The method of aspect 86, wherein the second time is from a period of time of from about 1 week to about 6 months from the first time.

Aspect 88. The method of any one of aspects 85-87, wherein said administering is intramuscular administration.

Aspect 89. The method of any one of aspects 85-87, wherein said administering is subcutaneous administration.

Aspect 90. The method of any one of aspects 85-89, comprising administering one or more heterologous polypeptides comprising a T-cell epitope present in an HCV protein other than E1 and E2.

Aspect 91. The method of any one of aspect 85-90, comprising administering an HCV E1 polypeptide and/or an HCV E2 polypeptide.

### Aspects Set B

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A composition comprising: a) one or more RNA molecules, wherein the one or more RNA molecules comprises: i) at least one 5′ cap structure and/or a 5′ untranslated region (5′ UTR) and/or a 5′ internal ribosome entry site (IRES); ii) a polypeptide-encoding region comprising one or more nucleotide sequences encoding a hepatitis C virus (HCV) E1 polypeptide and/or an HCV E2 polypeptide; and b) one or more of: i) a lipid; ii) a polymer; iii) a peptide; iv) a viral-like particle; and v) a cationic nanoemulsion.

Aspect 2. The composition of aspect 1, wherein the one or more RNA molecules comprises a 5′ cap structure and a 5′ UTR, wherein the 5′UTR is 3′ of the 5′ cap structure.

Aspect 3. The composition of aspect 1 or aspect 2, wherein the one or more RNA molecules comprises a 3′ UTR and/or a 3′ tailing sequence.

Aspect 4. The composition of aspect 3, wherein the 3′ tailing sequence is a poly(adenosine) (poly-A) sequence.

Aspect 5. The composition of any one of aspects 1-4, wherein the one or more RNA molecules include at least one 5′ cap structure, and wherein the at least one 5′ cap structure is selected from the group consisting of Cap0, Cap1, ARCA, inosine, N1-methyl-guanosine, 2′fluoro-guanosine, 7-deaza-guanosine, 8-oxo-guanosine, 2-amino-guanosine, LNA-guanosine, and 2-azido-guanosine.

Aspect 6. The composition of any one of aspects 1-5, wherein the one or more RNA molecules comprises one or more of: a nucleoside base modification, a sugar modification, and a backbone modification.

Aspect 7. The composition any one of aspects 1-6, wherein at least one of the one or more RNA molecules comprises a nucleotide of Formula I:

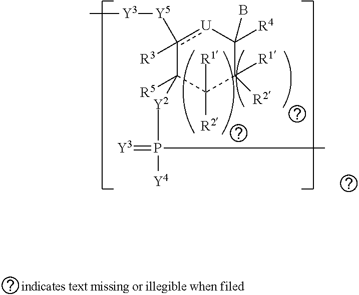

wherein:
U is O, S, N(R^{U})ₙᵤ, or C(R^{U})ₙᵤ, where nu is an integer nu is an integer from 0 to 2 and each R^{U} is, independently, H, halo, or optionally substituted alkyl;
[symbol] is a single or double bond;
[symbol] is a single bond or absent;
each of R^{1′}, R^{2′}, R^{1″}, R^{2″}, R³, R⁴, and R⁵ is, independently, H, halo, hydroxy, thiol, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, or absent; wherein the combination of R³ with one or more of R^{1′}, R^{1″}, R^{2′}, R^{2″}, or R⁵ can join together to form optionally substituted alkylene or optionally substituted heteroalkylene and, taken together with the carbons to which they are attached, provide an optionally substituted heterocyclyl; wherein the combination of R⁵ with one or more of R^{1′}, R^{1″}, R^{2′}, or R^{2″} can join together to form optionally substituted alkylene or optionally substituted heteroalkylene and, taken together with the carbons to which they are attached, provide an optionally substituted heterocyclyl; and wherein the combination of R⁴ and one or more of R^{1′}, R^{1″}, R^{2′}, R^{2″}, R³, or R⁵ can join together to form optionally substituted alkylene or optionally substituted heteroalkylene and, taken together with the carbons to which they are attached, provide an optionally substituted heterocyclyl;
each of Y¹, Y², and Y³, is, independently, O, S, —NR^{N1}—, optionally substituted alkylene, or optionally substituted heteroalkylene, wherein R^{N1} is H, optionally substituted alkyl, optionally substituted alkenyl, optionally substituted alkynyl, optionally substituted aryl, or absent;
each Y⁴ is, independently, H, hydroxy, thiol, optionally substituted alkyl, optionally substituted alkenyl, optionally substituted alkynyl, optionally substituted alkoxy, optionally substituted alkenyloxy, optionally substituted alkynyloxy, optionally substituted thioalkoxy, optionally substituted alkoxyalkoxy, or optionally substituted amino;
each Y⁵ is, independently, O, S, optionally substituted alkylene, or optionally substituted heteroalkylene;
n is an integer from 1 to 100,000; and
B is a nucleobase, wherein the combination of B and R^{1′}, the combination of B and R^{2′}, the combination of B and R^{1″}, or the combination of B and R^{2″} can, taken together with the carbons to which they are attached, optionally form a bicyclic group or wherein the combination of B, R^{1″}, and R³ or the combination of B, R^{2″}, and R³ can optionally form a tricyclic or tetracyclic group.

Aspect 8. The composition of any one of aspects 1-7, wherein the composition comprises a lipid, and wherein the lipid is selected from DLin-DMA, DLin-K-DMA, DLin-KC2-DMA, 98N12-5, C12-200, DLin-MC3-DMA, DODMA, DSDMA, DLenDMA, reLNPs, PLGA, a PEGylated lipid, and mixtures of two or more of the foregoing.

Aspect 9. The composition of any one of aspects 1-7, wherein the composition comprises a polymer.

Aspect 10. The composition of aspect 9, herein the polymer is selected from polyethylene glycol, polyglycolide, polyvinyl alcohol, polyvinyl pyrrolidone, polylactide, poly(lactide-co-glycolide) (PLGA), polycaprolactone, polysorbate, polyethylene oxide, polypropylene oxide, poly(ethylene oxide-co-propylene oxide), poloxamer, poloxamine, poly(oxyethylated) glycerol, poly(oxyethylated) sorbitol, poly(oxyethylated) glucose, polyethyleneimine, polyamidoamine (PAMAM) dendrimer, and block copolymer poly(ethylene glycol)-block-poly(lactic-co-glycolic acid) (PEG-b-PLGA).

Aspect 11. The composition of any one of aspects 1-10, wherein the HCV E1 polypeptide is a soluble HCV E1 polypeptide.

Aspect 12. The composition of any one of aspects 1-10, wherein the HCV E2 polypeptide is a soluble HCV E2 polypeptide.

Aspect 13. The composition of any one of aspects 1-12, wherein the nucleotide sequence encodes an HCV E1 polypeptide and an HCV E2 polypeptide.

Aspect 14. The composition of aspect 13, wherein:
a) the HCV E1 polypeptide is a soluble HCV E1 polypeptide;
b) the HCV E2 polypeptide is a soluble HCV E2 polypeptide; or
c) the HCV E1 polypeptide is a soluble HCV E1 polypeptide and the HCV E2 polypeptide is a soluble HCV E2 polypeptide.

Aspect 15. The composition of any one of aspects 1-14, wherein the polypeptide-encoding region comprises a nucleotide sequence encoding an HCV Core polypeptide.

Aspect 16. The composition of aspect 15, wherein the polypeptide-encoding region comprises a nucleotide sequence encoding an HCV Core-E1-E2 polyprotein.

Aspect 17. The composition of any one of aspects 1-16, wherein the polypeptide-encoding region comprises: a) one or more nucleotide sequences encoding one or more heterologous polypeptides comprising a T-cell epitope present in an HCV protein other than E1 and E2; or b) a nucleotide sequence encoding a fusion polypeptide comprising two or more heterologous polypeptides comprising a T-cell epitope present in an HCV protein other than E1 and E2.

Aspect 18. The composition of aspect 17, wherein the RNA comprises: a) an internal ribosome entry site (IRES) between i) the one or more nucleotide sequences encoding the HCV E1 and/or E2 polypeptides; and ii) the one or more nucleotide sequences encoding the one or more heterologous polypeptides or the nucleotide sequence encoding the fusion polypeptide; or b) a nucleotide sequence encoding a self-cleaving peptide between i) the one or more nucleotide sequences encoding the HCV E1 and/or E2 polypeptides; and ii) the one or more nucleotide sequences encoding the one or more heterologous polypeptides or the nucleotide sequence encoding the fusion polypeptide.

Aspect 19. The composition of aspect 17 or aspect 18, wherein the one or more heterologous polypeptides is selected from:
i) a TP35-NS3 T-cell epitope polypeptide comprising an amino acid sequence having at least 80% amino acid sequence identity to: KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
ii) a TP50C T-cell epitope polypeptide comprising an amino acid sequence having at least 80% amino acid sequence identity to: GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₂ is S, T, H, or A; X₁₂ is A or G; and X₁₃ is Q or K, wherein the TP50C T-cell epitope polypeptide has a length of from 40 amino acids to 50 amino acids;
iii) a TP23 T-cell epitope polypeptide comprising an amino acid sequence having at least 80% amino acid sequence identity to: DVVVX₁X₂TDALMTGX₃TGDFDSVID (SEQ ID NO:257), wherein X₁ is V or C; X₂ is A or S; and X₃ is F or Y, wherein the TP23 T-cell epitope polypeptide has a length of from 18 amino acids to 23 amino acids;
iv) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids;
v) a TP35-NS4 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀ (SEQ ID NO:288), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; and X₁₀ is V or I, wherein the TP35-NS4 T-cell epitope polypeptide has a length of from 28 amino acids to 35 amino acids;
vi) a TP42 T-cell epitope polypeptide comprising an amino acid sequence having at least 80% amino acid sequence identity to: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:314), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S, wherein n is an integer from 2 to 10, and wherein the TP42 T-cell epitope polypeptide has a length of from 34 amino acids to 52 amino acids;
vii) a TP45 T-cell epitope polypeptide comprising an amino acid sequence having at least 80% amino acid sequence identity to: X₁X₂AVAX₃YRGX₄DVX₅X₆IPX₇X₈GDVVVX₉X₁₀TDALMTGX₁₁TGDFDSVIDX₁₂X₁₃X₁₄(K)n (SEQ ID NO:332), wherein X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁ is F or Y; X₁₂ is C or K; X₃ is N or K; and X₁₄ is V or K, wherein n is an integer from 2 to 10, and wherein the TP45 T-cell epitope polypeptide has a length of from 36 amino acids to 55 amino acids; and
viii) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acids;
ix) a TP33 T-cell epitope polypeptide comprising an amino acid sequence having at least 80% amino acid sequence identity to: HSKKKCDELAX₁X₂LX₃X₄X₅GX₆NAVAYYRGLDVSX₇IP (SEQ ID NO:366), where X₁ is A or S; X₂ is K or A; X₃ is V, S, R, or T; X₄ is A or G; X₅ is L or M; X₆ is I, L, or V; and X₇ is V or I; wherein the TP33 T-cell epitope polypeptide has a length of from 30 amino acids to 36 amino acids;
x) a TP42-2 T-cell epitope polypeptide comprising an amino acid sequence having at least 80% amino acid sequence identity to: KGGRHLIFCHSKKKCDELAX₁X₂LX₃X₄X₅GX₆NAVAYYRGLDVSX₇IP (SEQ ID NO:371), where X₁ is A or S; X₂ is K or A; X₃ is V, S, R, or T; X₄ is A or G; X₅ is L or M; X₆ is I, L, or V; and X₇ is V or I; wherein the TP42-2 T-cell epitope polypeptide has a length of from 38 amino acids to 46 amino acids
xi) a TP240 T-cell epitope polypeptide comprising an amino acid sequence having at least 80% amino acid sequence identity to:

(SEQ ID NO: 379)
SYQVGYLHAPTGSGKSTKVPAAYAAQGYKVLVLNPSVAATLGFGAYMSK
AHGIDPNIRTGVRTVTTGAPITYSTYGKFLADGGCSGGAYDIIICDECH
SX₁DATTILGIGTVLDQAETAGARLVVLATATPPGSVTVPHPNIEEVAL
GX₂EGEIPFYGKAIPLX₃X₄IKGGRHLIFCHSKKKCDELAAKLRGMGLN
AVAYYRGLDVSVIPTX₅GDVVVVATDALMTGYTGDFDSVIDCNVAVTQT,

where X₁ is T, V, or Q, X₂ is T, N, Q, H, or S, X₃ is E, S, or A, X₄ is V, T, Y, F, or L, and X₅ is S, Q, or T; wherein the TP240 T-cell epitope polypeptide has a length of from 240 amino acids to 245 amino acids; and
xii) a TP65 T-cell epitope polypeptide comprising an amino acid sequence having at least 80% amino acid sequence identity to: TPIDTTIMAKNEVFCVDPX₆KGGRKPARLIVYPDLGVRVCEKMALYDVVQKLPQAVMGX₇SYGF QYS (SEQ ID NO:375), where X₆ is E, T, or V, and X₇ is S, A, or P; and wherein the TP65 T-cell epitope polypeptide has a length of from 65 amino acids to 70 amino acids.

Aspect 20. The composition of any one of aspects 17-19, wherein the polypeptide-encoding region comprises one or more nucleotide sequences encoding two or more heterologous polypeptides comprising a T-cell epitope present in an HCV protein other than E1 and E2.

Aspect 21. The composition of aspect 20, wherein the mRNA comprises: a) a first internal ribosome entry site (IRES) between i) the one or more nucleotide sequences encoding the HCV E1 and/or E2 polypeptides; and ii) the nucleotide sequence encoding the first of the two or more heterologous polypeptides; and b) a second IRES between i) the nucleotide sequence encoding the first of the two or more heterologous polypeptides; and ii) the nucleotide sequence encoding the second of the two or more heterologous polypeptides, wherein the first IRES and the second IRES are the same or different.

Aspect 22. The composition of aspect 21, wherein the mRNA comprises an IRES between any two consecutive nucleotide sequences encoding heterologous polypeptides.

Aspect 23. The composition of aspect 17 or aspect 18, wherein fusion polypeptide comprises from 2 to 10 polypeptides selected from the group consisting of:
i) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids; and
ii) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of from 40 amino acids to 50 amino acids;
iii) a TP23 T-cell epitope polypeptide comprising an amino acid sequence having at least 80% amino acid sequence identity to: DVVVX₁X₂TDALMTGX₃TGDFDSVID (SEQ ID NO:257), wherein X₁ is V or C; X₂ is A or S; and X₃ is F or Y, wherein the TP23 T-cell epitope polypeptide has a length of from 18 amino acids to 23 amino acids;
iv) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids;
v) a TP35-NS4 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀ (SEQ ID NO:288), wherein X₁ is I or V; X₂ is L or I; X₃ is R or K; X₄ is V, I, or T; X₅ is P, Q, or T; X₆ is G, A, or S; X₇ is A or V; X₅ is V or T; X₉ is S or A; and X₁₀ is V or I, wherein the TP35-NS4 T-cell epitope polypeptide has a length of from 28 amino acids to 35 amino acids;
vi) a TP42 T-cell epitope polypeptide comprising an amino acid sequence having at least 80% amino acid sequence identity to: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:314), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S, wherein n is an integer from 2 to 10, and wherein the TP42 T-cell epitope polypeptide has a length of from 34 amino acids to 52 amino acids;
vii) a TP45 T-cell epitope polypeptide comprising an amino acid sequence having at least 80% amino acid sequence identity to: X₁X₂AVAX₃YRGX₄DVX₅X₆IPX₇X₈GDVVVX₉X₁₀TDALMTGX₁₁TGDFDSVIDX₁₂X₁₃X₁₄(K)n (SEQ ID NO:332), wherein X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₂ is F or Y; X₁₂ is C or K; X₁₃ is N or K; and X₁₄ is V or K, wherein n is an integer from 2 to 10, and wherein the TP45 T-cell epitope polypeptide has a length of from 36 amino acids to 55 amino acids; and
viii) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acids;
ix) a TP33 T-cell epitope polypeptide comprising an amino acid sequence having at least 80% amino acid sequence identity to: HSKKKCDELAX₁X₂LX₃X₄X₅GX₆NAVAYYRGLDVSX₇IP (SEQ ID NO:366), where X₁ is A or S; X₂ is K or A; X₃ is V, S, R, or T; X₄ is A or G; X₅ is L or M; X₆ is I, L, or V; and X₇ is V or I; wherein the TP33 T-cell epitope polypeptide has a length of from 30 amino acids to 36 amino acids; and
x) a TP42-2 T-cell epitope polypeptide comprising an amino acid sequence having at least 80% amino acid sequence identity to: KGGRHLIFCHSKKKCDELAX₁X₂LX₃X₄X₅GX₆NAVAYYRGLDVSX₇IP (SEQ ID NO:371), where X₁ is A or S; X₂ is K or A; X₃ is V, S, R, or T; X₄ is A or G; X₅ is L or M; X₆ is I, L, or V; and X₇ is V or I; wherein the TP42-2 T-cell epitope polypeptide has a length of from 38 amino acids to 46 amino acids;
xi) a TP240 T-cell epitope polypeptide comprising an amino acid sequence having at least 80% amino acid sequence identity to:

(SEQ ID NO: 379)
SYQVGYLHAPTGSGKSTKVPAAYAAQGYKVLVLNPSVAATLGFGAYMSK
AHGIDPNIRTGVRTVTTGAPITYSTYGKFLADGGCSGGAYDIIICDECH
SX₁DATTILGIGTVLDQAETAGARLVVLATATPPGSVTVPHPNIEEVAL
GX₂EGEIPFYGKAIPLX₃X₄IKGGRHLIFCHSKKKCDELAAKLRGMGLN
AVAYYRGLDVSVIPTX₅GDVVVVATDALMTGYTGDFDSVIDCNVAVTQT,

where X₁ is T, V, or Q, X₂ is T, N, Q, H, or S, X₃ is E, S, or A, X₄ is V, T, Y, F, or L, and X₅ is S, Q, or T; wherein the TP240 T-cell epitope polypeptide has a length of from 240 amino acids to 245 amino acids; and
xii) a TP65 T-cell epitope polypeptide comprising an amino acid sequence having at least 80% amino acid sequence identity to: TPIDTTIMAKNEVFCVDPX₆KGGRKPARLIVYPDLGVRVCEKMALYDVVQKLPQAVMGX₇SYGF QYS (SEQ ID NO:375), where X₆ is E, T, or V, and X₇ is S, A, or P; and wherein the TP65 T-cell epitope polypeptide has a length of from 65 amino acids to 70 amino acids.

Aspect 24. The composition of any one of aspects 1-23, wherein the HCV E1 and/or E2 polypeptide is from HCV genotype 1.

Aspect 25. The composition of any one of aspects 1-23, wherein the HCV E1 and/or E2 polypeptide is from HCV genotype 2.

Aspect 26. The composition of any one of aspects 1-23, wherein the HCV E1 and/or E2 polypeptide is from HCV genotype 3.

Aspect 27. The composition of any one of aspects 1-23, wherein the HCV E1 and/or E2 polypeptide is/are from: a) HCV genotype 1, 2, and 3; b) HCV genotype 1a, 2, and 3; c) HCV genotype 1b, 2, and 3; or d) HCV genotype 1a, 1b, 2, and 3.

Aspect 28. The composition of any one of aspects 24-27, wherein the polypeptide-encoding region comprises a nucleotide sequence encoding an HCV Core polypeptide, wherein the HCV Core polypeptide is of genotype 1, genotype 2, or genotype 3.

Aspect 29. The composition of any one of aspects 17-28, wherein the one or more RNA molecules comprises a first RNA molecule, a second RNA molecule, and a third RNA molecule, wherein: a) the polypeptide-encoding region of the first RNA molecule comprises: i) a nucleotide sequence encoding HCV E1 and/or an HCV E2 polypeptide of a first genotype; ii) a first IRES; and iii) one or more nucleotide sequences encoding the one or more heterologous polypeptides or the fusion polypeptide; b) the polypeptide-encoding region of the second RNA molecule comprises: i) a nucleotide sequence encoding HCV E1 and/or an HCV E2 polypeptide of a second genotype; ii) a second IRES; and iii) one or more nucleotide sequences encoding the one or more heterologous polypeptides or the fusion polypeptide; and c) the polypeptide-encoding region of the third RNA molecule comprises: i) a nucleotide sequence encoding HCV E1 and/or an HCV E2 polypeptide of a third genotype; ii) a third IRES; and iii) one or more nucleotide sequences encoding the one or more heterologous polypeptides or the fusion polypeptide; wherein the first IRES, the second IRES, and the third IRES are the same or different.

Aspect 30. The composition of aspect 29, wherein: a) the HCV E1 polypeptide encoded by the first RNA molecule is a soluble HCV E1 polypeptide and/or the HCV E2 polypeptide encoded by the first RNA molecule is a soluble HCV E2 polypeptide; and/or b) the HCV E1 polypeptide encoded by the second RNA molecule is a soluble HCV E1 polypeptide and/or the HCV E2 polypeptide encoded by the second RNA molecule is a soluble HCV E2 polypeptide; and/or c) the HCV E1 polypeptide encoded by the third RNA molecule is a soluble HCV E1 polypeptide and/or the HCV E2 polypeptide encoded by the third RNA molecule is a soluble HCV E2 polypeptide.

Aspect 31. The composition of aspect 29 or aspect 30, wherein the polypeptide-encoding region of one or more of the first RNA molecule, the second RNA molecule, and the third RNA molecule comprises a nucleotide sequence encoding an HCV Core polypeptide.

Aspect 32. The composition of aspect 29 or aspect 30, wherein:
a) the polypeptide-encoding region of the first RNA molecule comprises:
   i) a nucleotide sequence encoding HCV E1 and HCV E2 polypeptides of genotype 1;
   ii) a first IRES; and
   iii) a nucleotide sequence encoding the fusion polypeptide;
b) the polypeptide-encoding region of the second RNA molecule comprises:
   i) a nucleotide sequence encoding HCV E1 and HCV E2 polypeptides of genotype 2;
   ii) a second IRES; and
   iii) a nucleotide sequence encoding the fusion polypeptide; and
c) the polypeptide-encoding region of the third RNA molecule comprises:
   i) a nucleotide sequence encoding HCV E1 and HCV E2 polypeptides of genotype 3;
   ii) a third IRES; and
   iii) a nucleotide sequence encoding the fusion polypeptide;
   wherein the first IRES, the second IRES, and the third IRES are the same or different.

Aspect 33. The composition of aspect 32, wherein:
a) the HCV E1 polypeptide of genotype 1 comprises an amino acid sequence having at least 90%, or at least 95%, amino acid sequence identity to an H77 E1 polypeptide, and the HCV E2 polypeptide of genotype 1 comprises an amino acid sequence having at least 90%, or at least 95%, amino acid sequence identity to an H77 E2 polypeptide;
b) the HCV E1 polypeptide of genotype 2 comprises an amino acid sequence having at least 90%, or at least 95%, amino acid sequence identity to a J6 E1 polypeptide, and the HCV E2 polypeptide of genotype 2 comprises an amino acid sequence having at least 90%, or at least 95%, amino acid sequence identity to a J6 E2 polypeptide; and
c) the HCV E1 polypeptide of genotype 3 comprises an amino acid sequence having at least 90%, or at least 95%, amino acid sequence identity to an S52 E1 polypeptide, and the HCV E2 polypeptide of genotype 3 comprises an amino acid sequence having at least 90%, or at least 95%, amino acid sequence identity to an S52 E2 polypeptide.

Aspect 34. The composition of aspect 32 or aspect 33, wherein the polypeptide-encoding region of one or more of the first RNA molecule, the second RNA molecule, and the third RNA molecule comprises a nucleotide sequence encoding an HCV Core polypeptide.

Aspect 35. The composition of aspect 34, wherein:
a) the polypeptide-encoding region of the first RNA molecule comprises:
   i) a nucleotide sequence encoding an HCV Core-E1-E2 polypeptide of genotype 1;
   ii) a first IRES; and
   iii) a nucleotide sequence encoding the fusion polypeptide;
b) the polypeptide-encoding region of the second RNA molecule comprises:
   i) a nucleotide sequence encoding an HCV Core-E1-E2 polypeptide of genotype 2;
   ii) a second IRES; and
   iii) a nucleotide sequence encoding the fusion polypeptide; and
c) the polypeptide-encoding region of the third RNA molecule comprises:
   i) a nucleotide sequence encoding an HCV Core-E1-E2 polypeptide of genotype 3;
   ii) a third IRES; and
   iii) a nucleotide sequence encoding the fusion polypeptide;
   wherein the first IRES, the second IRES, and the third IRES are the same or different.

Aspect 36. The composition of aspect 35, wherein:
a) the HCV E1 polypeptide of genotype 1 comprises an amino acid sequence having at least 90%, or at least 95%, amino acid sequence identity to an H77 E1 polypeptide, and the HCV E2 polypeptide of genotype 1 comprises an amino acid sequence having at least 90%, or at least 95%, amino acid sequence identity to an H77 E2 polypeptide;
b) the HCV E1 polypeptide of genotype 2 comprises an amino acid sequence having at least 95% amino acid sequence identity to a J6 E1 polypeptide, and the HCV E2 polypeptide of genotype 2 comprises an amino acid sequence having at least 90%, or at least 95%, amino acid sequence identity to a J6 E2 polypeptide; and
c) the HCV E1 polypeptide of genotype 3 comprises an amino acid sequence having at least 90%, or at least 95%, amino acid sequence identity to an S52 E1 polypeptide, and the HCV E2 polypeptide of genotype 3 comprises an amino acid sequence having at least 90%, or at least 95%, amino acid sequence identity to an S52 E2 polypeptide.

Aspect 37. The composition of any one of aspects 17 to 28, wherein:
a) the polypeptide-encoding region of the first RNA molecule comprises a nucleotide sequence encoding a fusion polypeptide comprising:
   i) HCV E1 and HCV2 E2 polypeptides of genotype 1; and
   ii) the fusion polypeptide comprising two or more heterologous polypeptides comprising a T-cell epitope present in an HCV protein other than E1 and E2;
b) the polypeptide-encoding region of the second RNA molecule comprises a nucleotide sequence encoding a fusion polypeptide comprising:
   i) HCV E1 and HCV2 E2 polypeptides of genotype 2; and
   ii) the fusion polypeptide comprising two or more heterologous polypeptides comprising a T-cell epitope present in an HCV protein other than E1 and E2; and
c) the polypeptide-encoding region of the third RNA molecule comprises a nucleotide sequence encoding a fusion polypeptide comprising:
   i) HCV E1 and HCV2 E2 polypeptides of genotype 3; and
   ii) the fusion polypeptide comprising two or more heterologous polypeptides comprising a T-cell epitope present in an HCV protein other than E1 and E2.

Aspect 38. The composition of aspect 37, wherein:
a) the HCV E1 polypeptide of genotype 1 comprises an amino acid sequence having at least 90%, or at least 95%, amino acid sequence identity to an H77 E1 polypeptide, and the HCV E2 polypeptide of genotype 1 comprises an amino acid sequence having at least 90%, or at least 95%, amino acid sequence identity to an H77 E2 polypeptide;
b) the HCV E1 polypeptide of genotype 2 comprises an amino acid sequence having at least 90%, or at least 95%, amino acid sequence identity to a J6 E1 polypeptide, and the HCV E2 polypeptide of genotype 2 comprises an amino acid sequence having at least 90%, or at least 95%, amino acid sequence identity to a J6 E2 polypeptide; and
c) the HCV E1 polypeptide of genotype 3 comprises an amino acid sequence having at least 90%, or at least 95%, amino acid sequence identity to an S52 E1 polypeptide, and the HCV E2 polypeptide of genotype 3 comprises an amino acid sequence having at least 90%, or at least 95%, amino acid sequence identity to an S52 E2 polypeptide.

Aspect 39. The composition of aspect 37, wherein:
a) the HCV E1 polypeptide encoded by the first RNA molecule is a soluble HCV E1 polypeptide and/or the HCV E2 polypeptide encoded by the first RNA molecule is a soluble HCV E2 polypeptide; and/or
b) the HCV E1 polypeptide encoded by the second RNA molecule is a soluble HCV E1 polypeptide and/or the HCV E2 polypeptide encoded by the second RNA molecule is a soluble HCV E2 polypeptide; and/or
c) the HCV E1 polypeptide encoded by the third RNA molecule is a soluble HCV E1 polypeptide and/or the HCV E2 polypeptide encoded by the third RNA molecule is a soluble HCV E2 polypeptide.

Aspect 40. The composition of aspect 39, wherein:
a) the soluble HCV E1 polypeptide of genotype 1 comprises an amino acid sequence having at least 90%, or at least 95%, amino acid sequence identity to a soluble H77 E1 polypeptide, and the soluble HCV E2 polypeptide of genotype 1 comprises an amino acid sequence having at least 90%, or at least 95%, amino acid sequence identity to a soluble H77 E2 polypeptide;
b) the soluble HCV E1 polypeptide of genotype 2 comprises an amino acid sequence having at least 90%, or at least 95%, amino acid sequence identity to a soluble J6 E1 polypeptide, and the soluble HCV E2 polypeptide of genotype 2 comprises an amino acid sequence having at least 90%, or at least 95%, amino acid sequence identity to a soluble J6 E2 polypeptide; and
c) the soluble HCV E1 polypeptide of genotype 3 comprises an amino acid sequence having at least 90%, or at least 95%, amino acid sequence identity to a soluble S52 E1 polypeptide, and the soluble HCV E2 polypeptide of genotype 3 comprises an amino acid sequence having at least 90%, or at least 95%, amino acid sequence identity to a soluble S52 E2 polypeptide.

Aspect 41. The composition of any one of aspects 37-40, wherein the polypeptide-encoding region of one or more of the first RNA molecule, the second RNA molecule, and the third RNA molecule comprises a nucleotide sequence encoding an HCV Core polypeptide.

Aspect 42. The composition of aspect 41, wherein:
a) the polypeptide-encoding region of the first RNA molecule comprises a nucleotide sequence encoding a fusion polypeptide comprising:
   i) an HCV Core-E1-E2 polyprotein of genotype 1; and
   ii) the fusion polypeptide comprising two or more heterologous polypeptides comprising a T-cell epitope present in an HCV protein other than E1 and E2;
b) the polypeptide-encoding region of the second RNA molecule comprises a nucleotide sequence encoding a fusion polypeptide comprising:
   i) an HCV Core-E1-E2 polyprotein of genotype 2; and
   ii) the fusion polypeptide comprising two or more heterologous polypeptides comprising a T-cell epitope present in an HCV protein other than E1 and E2; and
c) the polypeptide-encoding region of the third RNA molecule comprises a nucleotide sequence encoding a fusion polypeptide comprising:
   i) an HCV Core-E1-E2 polyprotein of genotype 3; and
   ii) the fusion polypeptide comprising two or more heterologous polypeptides comprising a T-cell epitope present in an HCV protein other than E1 and E2.

Aspect 43. The composition of aspect 42, wherein:
a) the HCV Core-E1-E2 polyprotein of genotype 1 comprises an amino acid sequence having at least 90%, or at least 95%, amino acid sequence identity to an H77 Core-E1-E2 polyprotein;
b) the HCV Core-E1-E2 polyprotein of genotype 2 comprises an amino acid sequence having at least 90%, or at least 95%, amino acid sequence identity to a J6 Core-E1-E2 polyprotein; and
c) the HCV Core-E1-E2 polyprotein of genotype 3 comprises an amino acid sequence having at least 90%, or at least 95%, amino acid sequence identity to an S52 Core-E1-E2 polyprotein.

44. The composition of claim 41, wherein: a) the polypeptide-encoding region of the first RNA molecule comprises a nucleotide sequence encoding a fusion polypeptide comprising: i) an HCV Core-E1-E2 polyprotein of genotype 1; and ii) the fusion polypeptide comprising two or more heterologous polypeptides comprising a T-cell epitope present in an HCV protein other than E1 and E2; b) the polypeptide-encoding region of the second RNA molecule comprises a nucleotide sequence encoding a fusion polypeptide comprising: i) an HCV Core-E1-E2 polyprotein of genotype 3; and ii) the fusion polypeptide comprising two or more heterologous polypeptides comprising a T-cell epitope present in an HCV protein other than E1 and E2; and c) the polypeptide-encoding region of the third RNA molecule comprises a nucleotide sequence encoding a fusion polypeptide comprising: i) an HCV Core-E1-E2 polyprotein of genotype 4, genotype 5, or genotype 6; and ii) the fusion polypeptide comprising two or more heterologous polypeptides comprising a T-cell epitope present in an HCV protein other than E1 and E2.

Aspect 45. The composition of any one of aspects 23-44, wherein the fusion polypeptide comprising two or more heterologous polypeptides comprising a T-cell epitope present in an HCV protein other than E1 and E2 comprises:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; Xⱼ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID N0219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 46. The composition of any one of aspects 23-45, wherein the fusion polypeptide comprising two or more heterologous polypeptides comprising a T-cell epitope present in an HCV protein other than E1 and E2 comprises, in order from N-terminus to C-terminus:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 47. The composition of any one of aspects 17-28, wherein the one or more RNA molecules comprises a first RNA molecule and a second RNA molecule, wherein: a) the polypeptide-encoding region of the first RNA molecule comprises: i) a nucleotide sequence encoding an HCV E1 and/or an HCV E2 polypeptide of a first genotype; ii) a first IRES; and iii) one or more nucleotide sequences encoding the one or more heterologous polypeptides or the fusion polypeptide; and b) the polypeptide-encoding region of the second RNA molecule comprises: i) a nucleotide sequence encoding an HCV E1 and/or an HCV E2 polypeptide of a second genotype; ii) a second IRES; and iii) one or more nucleotide sequences encoding the one or more heterologous polypeptides or the fusion polypeptide; wherein the first IRES and the second IRES are the same or different.

Aspect 48. The composition of aspect 47, wherein: a) the HCV E1 polypeptide encoded by the first RNA molecule is a soluble HCV E1 polypeptide and/or the HCV E2 polypeptide encoded by the first RNA molecule is a soluble HCV E2 polypeptide; and/or b) the HCV E1 polypeptide encoded by the second RNA molecule is a soluble HCV E1 polypeptide and/or the HCV E2 polypeptide encoded by the second RNA molecule is a soluble HCV E2 polypeptide.

Aspect 49. The composition of aspect 47 or 48, wherein the polypeptide-encoding region of one or both of the first RNA molecule and the second RNA molecule comprises a nucleotide sequence encoding an HCV Core polypeptide.

Aspect 50. The composition of aspect 47 or 48, wherein:
a1) the polypeptide-encoding region of the first RNA molecule comprises: i) a nucleotide sequence encoding HCV E1 and HCV E2 polypeptides of genotype 1; ii) a first IRES; and iii) a nucleotide sequence encoding the fusion polypeptide; and
b1) the polypeptide-encoding region of the second RNA molecule comprises: i) a nucleotide sequence encoding HCV E1 and HCV E2 polypeptides of genotype 3; ii) a second IRES; and iii) a nucleotide sequence encoding the fusion polypeptide; or
a2) the polypeptide-encoding region of the first RNA molecule comprises: i) a nucleotide sequence encoding HCV E1 and HCV E2 polypeptides of genotype 1; ii) a first IRES; and iii) a nucleotide sequence encoding the fusion polypeptide; and
b2) the polypeptide-encoding region of the second RNA molecule comprises: i) a nucleotide sequence encoding HCV E1 and HCV E2 polypeptides of genotype 2; ii) a second IRES; and iii) a nucleotide sequence encoding the fusion polypeptide; or
a3) the polypeptide-encoding region of the first RNA molecule comprises: i) a nucleotide sequence encoding HCV E1 and HCV E2 polypeptides of genotype 2; ii) a first IRES; and iii) a nucleotide sequence encoding the fusion polypeptide; and
b3) the polypeptide-encoding region of the second RNA molecule comprises: i) a nucleotide sequence encoding HCV E1 and HCV E2 polypeptides of genotype 3; ii) a second IRES; and iii) a nucleotide sequence encoding the fusion polypeptide;
wherein the first IRES and the second IRES are the same or different.

Aspect 51. The composition of aspect 50, wherein:
a) the HCV E1 polypeptide of genotype 1 comprises an amino acid sequence having at least 90%, or at least 95%, amino acid sequence identity to an H77 E1 polypeptide, and the HCV E2 polypeptide of genotype 1 comprises an amino acid sequence having at least 90%, or at least 95%, amino acid sequence identity to an H77 E2 polypeptide;
b) the HCV E1 polypeptide of genotype 2 comprises an amino acid sequence having at least 90%, or at least 95%, amino acid sequence identity to a J6 E1 polypeptide, and the HCV E2 polypeptide of genotype 2 comprises an amino acid sequence having at least 90%, or at least 95%, amino acid sequence identity to a J6 E2 polypeptide; and
c) the HCV E1 polypeptide of genotype 3 comprises an amino acid sequence having at least 90%, or at least 95%, amino acid sequence identity to an S52 E1 polypeptide, and the HCV E2 polypeptide of genotype 3 comprises an amino acid sequence having at least 90%, or at least 95%, amino acid sequence identity to an S52 E2 polypeptide.

Aspect 52. The composition of aspect 50 or aspect 51, wherein the polypeptide-encoding region of one or both of the first RNA molecule and the second RNA molecule comprises a nucleotide sequence encoding an HCV Core polypeptide.

Aspect 53. The composition of aspect 52, wherein:
a1) the polypeptide-encoding region of the first RNA molecule comprises: i) a nucleotide sequence encoding an HCV Core-E1-E2 polyprotein of genotype 1; ii) a first IRES; and iii) a nucleotide sequence encoding the fusion polypeptide;
b1) the polypeptide-encoding region of the second RNA molecule comprises: i) a nucleotide sequence encoding an HCV Core-E1-E2 polyprotein of genotype 3; ii) a second IRES; and iii) a nucleotide sequence encoding the fusion polypeptide; or
a2) the polypeptide-encoding region of the first RNA molecule comprises: i) a nucleotide sequence encoding an HCV Core-E1-E2 polyprotein of genotype 1; ii) a first IRES; and iii) a nucleotide sequence encoding the fusion polypeptide;
b2) the polypeptide-encoding region of the second RNA molecule comprises: i) a nucleotide sequence encoding an HCV Core-E1-E2 polyprotein of genotype 2; ii) a second IRES; and iii) a nucleotide sequence encoding the fusion polypeptide; or
a3) the polypeptide-encoding region of the first RNA molecule comprises: i) a nucleotide sequence encoding an HCV Core-E1-E2 polyprotein of genotype 2; ii) a first IRES; and iii) a nucleotide sequence encoding the fusion polypeptide;
b3) the polypeptide-encoding region of the second RNA molecule comprises: i) a nucleotide sequence encoding an HCV Core-E1-E2 polyprotein of genotype 3; ii) a second IRES; and iii) a nucleotide sequence encoding the fusion polypeptide; wherein the first IRES and the second IRES are the same or different.

Aspect 54. The composition of aspect 53, wherein:
a) the HCV Core-E1-E2 polyprotein of genotype 1 comprises an amino acid sequence having at least 90%, or at least 95%, amino acid sequence identity to an H77 HCV Core-E1-E2 polyprotein;
b) the HCV Core-E1-E2 polyprotein of genotype 2 comprises an amino acid sequence having at least 90%, or at least 95%, amino acid sequence identity to a J6 HCV Core-E1-E2 polyprotein; and
c) the HCV Core-E1-E2 polyprotein of genotype 3 comprises an amino acid sequence at least 90%, or at least 95%, amino acid sequence identity to an S52 HCV Core-E1-E2 polyprotein.

Aspect 55. The composition of any one of aspects 17 to 28, wherein the composition comprises a first RNA molecule and a second RNA molecule, and wherein:
a1) the polypeptide-encoding region of the first RNA molecule comprises a nucleotide sequence encoding a fusion polypeptide comprising: i) HCV E1 and HCV2 E2 polypeptides of genotype 1; and ii) the fusion polypeptide comprising two or more heterologous polypeptides comprising a T-cell epitope present in an HCV protein other than E1 and E2;
b1) the polypeptide-encoding region of the second RNA molecule comprises a nucleotide sequence encoding a fusion polypeptide comprising: i) HCV E1 and HCV2 E2 polypeptides of genotype 3; and ii) the fusion polypeptide comprising two or more heterologous polypeptides comprising a T-cell epitope present in an HCV protein other than E1 and E2; or
a2) the polypeptide-encoding region of the first RNA molecule comprises a nucleotide sequence encoding a fusion polypeptide comprising: i) HCV E1 and HCV2 E2 polypeptides of genotype 1; and ii) the fusion polypeptide comprising two or more heterologous polypeptides comprising a T-cell epitope present in an HCV protein other than E1 and E2;
b2) the polypeptide-encoding region of the second RNA molecule comprises a nucleotide sequence encoding a fusion polypeptide comprising: i) HCV E1 and HCV2 E2 polypeptides of genotype 2; and ii) the fusion polypeptide comprising two or more heterologous polypeptides comprising a T-cell epitope present in an HCV protein other than E1 and E2; or
a3) the polypeptide-encoding region of the first RNA molecule comprises a nucleotide sequence encoding a fusion polypeptide comprising: i) HCV E1 and HCV2 E2 polypeptides of genotype 2; and ii) the fusion polypeptide comprising two or more heterologous polypeptides comprising a T-cell epitope present in an HCV protein other than E1 and E2;
b3) the polypeptide-encoding region of the second RNA molecule comprises a nucleotide sequence encoding a fusion polypeptide comprising: i) HCV E1 and HCV2 E2 polypeptides of genotype 3; and ii) the fusion polypeptide comprising two or more heterologous polypeptides comprising a T-cell epitope present in an HCV protein other than E1 and E2.

Aspect 56. The composition of aspect 55, wherein:
a) the HCV E1 polypeptide of genotype 1 comprises an amino acid sequence having at least 90%, or at least 95%, amino acid sequence identity to an H77 E1 polypeptide, and the HCV E2 polypeptide of genotype 1 comprises an amino acid sequence having at least 90%, or at least 95%, amino acid sequence identity to an H77 E2 polypeptide;
b) the HCV E1 polypeptide of genotype 2 comprises an amino acid sequence having at least 90%, or at least 95%, amino acid sequence identity to a J6 E1 polypeptide, and the HCV E2 polypeptide of genotype 2 comprises an amino acid sequence having at least 90%, or at least 95%, amino acid sequence identity to a J6 E2 polypeptide; and
c) the HCV E1 polypeptide of genotype 3 comprises an amino acid sequence having at least 90%, or at least 95%, amino acid sequence identity to an S52 E1 polypeptide, and the HCV E2 polypeptide of genotype 3 comprises an amino acid sequence having at least 90%, or at least 95%, amino acid sequence identity to an S52 E2 polypeptide.

Aspect 57. The composition of aspect 55, wherein:
a) the HCV E1 polypeptide encoded by the first RNA molecule is a soluble HCV E1 polypeptide and/or the HCV E2 polypeptide encoded by the first RNA molecule is a soluble HCV E2 polypeptide; and/or
b) the HCV E1 polypeptide encoded by the second RNA molecule is a soluble HCV E1 polypeptide and/or the HCV E2 polypeptide encoded by the second RNA molecule is a soluble HCV E2 polypeptide.

Aspect 58. The composition of aspect 57, wherein:
a) the soluble HCV E1 polypeptide of genotype 1 comprises an amino acid sequence having at least 90%, or at least 95%, amino acid sequence identity to a soluble H77 E1 polypeptide, and the soluble HCV E2 polypeptide of genotype 1 comprises an amino acid sequence having at least 90%, or at least 95%, amino acid sequence identity to a soluble H77 E2 polypeptide;
b) the soluble HCV E1 polypeptide of genotype 2 comprises an amino acid sequence having at least 90%, or at least 95%, amino acid sequence identity to a soluble J6 E1 polypeptide, and the soluble HCV E2 polypeptide of genotype 2 comprises an amino acid sequence having at least 90%, or at least 95%, amino acid sequence identity to a soluble J6 E2 polypeptide; and
c) the soluble HCV E1 polypeptide of genotype 3 comprises an amino acid sequence having at least 90%, or at least 95%, amino acid sequence identity to a soluble S52 E1 polypeptide, and the soluble HCV E2 polypeptide of genotype 3 comprises an amino acid sequence having at least 90%, or at least 95%, amino acid sequence identity to a soluble S52 E2 polypeptide.

Aspect 59. The composition of any one of aspects 55-58, wherein the polypeptide-encoding region of one or both of the first RNA molecule and the second RNA molecule comprises a nucleotide sequence encoding an HCV Core polypeptide.

Aspect 60. The composition of aspect 59, wherein:
a1) the polypeptide-encoding region of the first RNA molecule comprises a nucleotide sequence encoding a fusion polypeptide comprising: i) an HCV Core-E1-E2 polyprotein of genotype 1; and ii) the fusion polypeptide comprising two or more heterologous polypeptides comprising a T-cell epitope present in an HCV protein other than E1 and E2;
b1) the polypeptide-encoding region of the first RNA molecule comprises a nucleotide sequence encoding a fusion polypeptide comprising: i) an HCV Core-E1-E2 polyprotein of genotype 3; and ii) the fusion polypeptide comprising two or more heterologous polypeptides comprising a T-cell epitope present in an HCV protein other than E1 and E2; or
α2) the polypeptide-encoding region of the first RNA molecule comprises a nucleotide sequence encoding a fusion polypeptide comprising: i) an HCV Core-E1-E2 polyprotein of genotype 1; and ii) the fusion polypeptide comprising two or more heterologous polypeptides comprising a T-cell epitope present in an HCV protein other than E1 and E2;
b2) the polypeptide-encoding region of the first RNA molecule comprises a nucleotide sequence encoding a fusion polypeptide comprising: i) an HCV Core-E1-E2 polyprotein of genotype 2; and ii) the fusion polypeptide comprising two or more heterologous polypeptides comprising a T-cell epitope present in an HCV protein other than E1 and E2; or
a3) the polypeptide-encoding region of the first RNA molecule comprises a nucleotide sequence encoding a fusion polypeptide comprising: i) an HCV Core-E1-E2 polyprotein of genotype 2; and ii) the fusion polypeptide comprising two or more heterologous polypeptides comprising a T-cell epitope present in an HCV protein other than E1 and E2;
b3) the polypeptide-encoding region of the first RNA molecule comprises a nucleotide sequence encoding a fusion polypeptide comprising: i) an HCV Core-E1-E2 polyprotein of genotype 3; and ii) the fusion polypeptide comprising two or more heterologous polypeptides comprising a T-cell epitope present in an HCV protein other than E1 and E2.

Aspect 61. The composition of aspect 60, wherein:
a) the HCV Core-E1-E2 polyprotein of genotype 1 comprises an amino acid sequence having at least 90%, or at least 95%, amino acid sequence identity to an H77 Core-E1-E2 polyprotein;
b) the HCV Core-E1-E2 polyprotein of genotype 2 comprises an amino acid sequence having at least 90%, or at least 95%, amino acid sequence identity to a J6 Core-E1-E2 polyprotein; and
c) the HCV Core-E1-E2 polyprotein of genotype 3 comprises an amino acid sequence having at least 90%, or at least 95%, amino acid sequence identity to an S52 Core-E1-E2 polyprotein.

Aspect 62. The composition of any one of aspects 47-61, wherein the fusion polypeptide comprising two or more heterologous polypeptides comprising a T-cell epitope present in an HCV protein other than E1 and E2 comprises:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; Xⱼ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 63. The composition of any one of aspects 47-61, wherein the fusion polypeptide comprising two or more heterologous polypeptides comprising a T-cell epitope present in an HCV protein other than E1 and E2 comprises, in order from N-terminus to C-terminus:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 64. The composition of any one of aspects 17-28, wherein the composition comprises a single type of RNA molecule, wherein:
a) the polypeptide-encoding region of the RNA molecule comprises: i) a nucleotide sequence encoding an HCV E1 and/or an HCV E2 polypeptide of genotype 1; ii) a first IRES; and iii) one or more nucleotide sequences encoding the one or more heterologous polypeptides or the fusion polypeptide; or
b) the polypeptide-encoding region of the RNA molecule comprises: i) a nucleotide sequence encoding an HCV E1 and/or an HCV E2 polypeptide of genotype 2; ii) a first IRES; and iii) one or more nucleotide sequences encoding the one or more heterologous polypeptides or the fusion polypeptide; or
c) the polypeptide-encoding region of the RNA molecule comprises: i) a nucleotide sequence encoding an HCV E1 and/or an HCV E2 polypeptide of genotype 3; ii) a first IRES; and iii) one or more nucleotide sequences encoding the one or more heterologous polypeptides or the fusion polypeptide; or
d) the polypeptide-encoding region of the RNA molecule comprises: i) a nucleotide sequence encoding an HCV E1 and/or an HCV E2 polypeptide of genotype 4; ii) a first IRES; and iii) one or more nucleotide sequences encoding the one or more heterologous polypeptides or the fusion polypeptide; or
e) the polypeptide-encoding region of the RNA molecule comprises: i) a nucleotide sequence encoding an HCV E1 and/or an HCV E2 polypeptide of genotype 5; ii) a first IRES; and iii) one or more nucleotide sequences encoding the one or more heterologous polypeptides or the fusion polypeptide; or
f) the polypeptide-encoding region of the RNA molecule comprises: i) a nucleotide sequence encoding an HCV E1 and/or an HCV E2 polypeptide of genotype 6; ii) a first IRES; and iii) one or more nucleotide sequences encoding the one or more heterologous polypeptides or the fusion polypeptide.

Aspect 65. The composition of aspect 64, wherein:
a) the HCV E1 polypeptide of genotype 1 comprises an amino acid sequence having at least 90%, or at least 95%, amino acid sequence identity to an H77 E1 polypeptide, and the HCV E2 polypeptide of genotype 1 comprises an amino acid sequence having at least 90%, or at least 95%, amino acid sequence identity to an H77 E2 polypeptide;
b) the HCV E1 polypeptide of genotype 2 comprises an amino acid sequence having at least 90%, or at least 95%, % amino acid sequence identity to a J6 E1 polypeptide, and the HCV E2 polypeptide of genotype 2 comprises an amino acid sequence having at least 90%, or at least 95%, amino acid sequence identity to a J6 E2 polypeptide; and
c) the HCV E1 polypeptide of genotype 3 comprises an amino acid sequence having at least 90%, or at least 95%, amino acid sequence identity to an S52 E1 polypeptide, and the HCV E2 polypeptide of genotype 3 comprises an amino acid sequence having at least 90%, or at least 95%, amino acid sequence identity to an S52 E2 polypeptide.

Aspect 66. The composition of aspect 64 or aspect 65, wherein the polypeptide-encoding region of the RNA molecule comprises a nucleotide sequence encoding an HCV Core polypeptide.

Aspect 67. The composition of aspect 66, wherein:
a) the polypeptide-encoding region of the RNA molecule comprises: i) a nucleotide sequence encoding an HCV Core-E1-E2 polyprotein of genotype 1; ii) a first IRES; and iii) one or more nucleotide sequences encoding the one or more heterologous polypeptides or the fusion polypeptide; or
b) the polypeptide-encoding region of the RNA molecule comprises: i) a nucleotide sequence encoding an HCV Core-E1-E2 polyprotein of genotype 2; ii) a first IRES; and iii) one or more nucleotide sequences encoding the one or more heterologous polypeptides or the fusion polypeptide; or
c) the polypeptide-encoding region of the RNA molecule comprises: i) a nucleotide sequence encoding an HCV Core-E1-E2 polyprotein of genotype 3; ii) a first IRES; and iii) one or more nucleotide sequences encoding the one or more heterologous polypeptides or the fusion polypeptide; or
d) the polypeptide-encoding region of the RNA molecule comprises: i) a nucleotide sequence encoding an HCV Core-E1-E2 polyprotein of genotype 4; ii) a first IRES; and iii) one or more nucleotide sequences encoding the one or more heterologous polypeptides or the fusion polypeptide; or
e) the polypeptide-encoding region of the RNA molecule comprises: i) a nucleotide sequence encoding an HCV Core-E1-E2 polyprotein of genotype 5; ii) a first IRES; and iii) one or more nucleotide sequences encoding the one or more heterologous polypeptides or the fusion polypeptide; or
f) the polypeptide-encoding region of the RNA molecule comprises: i) a nucleotide sequence encoding an HCV Core-E1-E2 polyprotein of genotype 6; ii) a first IRES; and iii) one or more nucleotide sequences encoding the one or more heterologous polypeptides or the fusion polypeptide.

Aspect 68. The composition of aspect 67, wherein:
a) the HCV Core-E1-E2 polyprotein of genotype 1 comprises an amino acid sequence having at least 90%, or at least 95%, amino acid sequence identity to an H77 HCV Core-E1-E2 polyprotein;
b) the HCV Core-E1-E2 polyprotein of genotype 2 comprises an amino acid sequence having at least 90%, or at least 95%, amino acid sequence identity to a J6 HCV Core-E1-E2 polyprotein; and
c) the HCV Core-E1-E2 polyprotein of genotype 3 comprises an amino acid sequence having at least 90%, or at least 95%, amino acid sequence identity to an S52 HCV Core-E1-E2 polyprotein.

Aspect 69. The composition of any one of aspects 17 to 28, wherein the composition comprises a single type of RNA molecule, and wherein:
a) the polypeptide-encoding region of the RNA molecule comprises a nucleotide sequence encoding a fusion polypeptide comprising: i) HCV E1 and HCV2 E2 polypeptides of genotype 1; and ii) the fusion polypeptide comprising two or more heterologous polypeptides comprising a T-cell epitope present in an HCV protein other than E1 and E2; or
b) the polypeptide-encoding region of the RNA molecule comprises a nucleotide sequence encoding a fusion polypeptide comprising: i) HCV E1 and HCV2 E2 polypeptides of genotype 2; and ii) the fusion polypeptide comprising two or more heterologous polypeptides comprising a T-cell epitope present in an HCV protein other than E1 and E2; or
c) the polypeptide-encoding region of the RNA molecule comprises a nucleotide sequence encoding a fusion polypeptide comprising: i) HCV E1 and HCV2 E2 polypeptides of genotype 3; and ii) the fusion polypeptide comprising two or more heterologous polypeptides comprising a T-cell epitope present in an HCV protein other than E1 and E2; or
d) the polypeptide-encoding region of the RNA molecule comprises a nucleotide sequence encoding a fusion polypeptide comprising: i) HCV E1 and HCV2 E2 polypeptides of genotype 4; and ii) the fusion polypeptide comprising two or more heterologous polypeptides comprising a T-cell epitope present in an HCV protein other than E1 and E2; or
e) the polypeptide-encoding region of the RNA molecule comprises a nucleotide sequence encoding a fusion polypeptide comprising: i) HCV E1 and HCV2 E2 polypeptides of genotype 5; and ii) the fusion polypeptide comprising two or more heterologous polypeptides comprising a T-cell epitope present in an HCV protein other than E1 and E2; or
f) the polypeptide-encoding region of the RNA molecule comprises a nucleotide sequence encoding a fusion polypeptide comprising: i) HCV E1 and HCV2 E2 polypeptides of genotype 6; and ii) the fusion polypeptide comprising two or more heterologous polypeptides comprising a T-cell epitope present in an HCV protein other than E1 and E2.

Aspect 70. The composition of aspect 69, wherein:
a) the HCV E1 polypeptide of genotype 1 comprises an amino acid sequence having at least 90%, or at least 95%, amino acid sequence identity to an H77 E1 polypeptide, and the HCV E2 polypeptide of genotype 1 comprises an amino acid sequence having at least 90%, or at least 95%, amino acid sequence identity to an H77 E2 polypeptide;
b) the HCV E1 polypeptide of genotype 2 comprises an amino acid sequence having at least 90%, or at least 95%, amino acid sequence identity to a J6 E1 polypeptide, and the HCV E2 polypeptide of genotype 2 comprises an amino acid sequence having at least 90%, or at least 95%, amino acid sequence identity to a J6 E2 polypeptide; and
c) the HCV E1 polypeptide of genotype 3 comprises an amino acid sequence having at least 90%, or at least 95%, amino acid sequence identity to an S52 E1 polypeptide, and the HCV E2 polypeptide of genotype 3 comprises an amino acid sequence having at least 90%, or at least 95%, amino acid sequence identity to an S52 E2 polypeptide.

Aspect 71. The composition of aspect 69, wherein:
a) the HCV E1 polypeptide encoded by the RNA molecule is a soluble HCV E1 polypeptide; and/or
b) the HCV E2 polypeptide encoded by the RNA molecule is a soluble HCV E2 polypeptide.

Aspect 72. The composition of aspect 71, wherein:
a) the soluble HCV E1 polypeptide of genotype 1 comprises an amino acid sequence having at least 90%, or at least 95%, amino acid sequence identity to a soluble H77 E1 polypeptide, and the soluble HCV E2 polypeptide of genotype 1 comprises an amino acid sequence having at least 90%, or at least 95%, amino acid sequence identity to a soluble H77 E2 polypeptide;
b) the soluble HCV E1 polypeptide of genotype 2 comprises an amino acid sequence having at least 90%, or at least 95%, amino acid sequence identity to a soluble J6 E1 polypeptide, and the soluble HCV E2 polypeptide of genotype 2 comprises an amino acid sequence having at least 90%, or at least 95%, amino acid sequence identity to a soluble J6 E2 polypeptide; and
c) the soluble HCV E1 polypeptide of genotype 3 comprises an amino acid sequence having at least 90%, or at least 95%, amino acid sequence identity to a soluble S52 E1 polypeptide, and the soluble HCV E2 polypeptide of genotype 3 comprises an amino acid sequence having at least 90%, or at least 95%, amino acid sequence identity to a soluble S52 E2 polypeptide.

Aspect 73. The composition of any one of aspects 69-72, wherein the polypeptide-encoding region of the RNA molecule comprises a nucleotide sequence encoding an HCV Core polypeptide.

Aspect 74. The composition of aspect 73, wherein:
a) the polypeptide-encoding region of the RNA molecule comprises a nucleotide sequence encoding a fusion polypeptide comprising: i) an HCV Core-E1-E2 polyprotein of genotype 1; and ii) the fusion polypeptide comprising two or more heterologous polypeptides comprising a T-cell epitope present in an HCV protein other than E1 and E2; or
b) the polypeptide-encoding region of the RNA molecule comprises a nucleotide sequence encoding a fusion polypeptide comprising: i) an HCV Core-E1-E2 polyprotein of genotype 2; and ii) the fusion polypeptide comprising two or more heterologous polypeptides comprising a T-cell epitope present in an HCV protein other than E1 and E2; or
c) the polypeptide-encoding region of the RNA molecule comprises a nucleotide sequence encoding a fusion polypeptide comprising: i) an HCV Core-E1-E2 polyprotein of genotype 3; and ii) the fusion polypeptide comprising two or more heterologous polypeptides comprising a T-cell epitope present in an HCV protein other than E1 and E2; or
d) the polypeptide-encoding region of the RNA molecule comprises a nucleotide sequence encoding a fusion polypeptide comprising: i) an HCV Core-E1-E2 polyprotein of genotype 4; and ii) the fusion polypeptide comprising two or more heterologous polypeptides comprising a T-cell epitope present in an HCV protein other than E1 and E2; or
e) the polypeptide-encoding region of the RNA molecule comprises a nucleotide sequence encoding a fusion polypeptide comprising: i) an HCV Core-E1-E2 polyprotein of genotype 5; and ii) the fusion polypeptide comprising two or more heterologous polypeptides comprising a T-cell epitope present in an HCV protein other than E1 and E2; or
f) the polypeptide-encoding region of the RNA molecule comprises a nucleotide sequence encoding a fusion polypeptide comprising: i) an HCV Core-E1-E2 polyprotein of genotype 6; and ii) the fusion polypeptide comprising two or more heterologous polypeptides comprising a T-cell epitope present in an HCV protein other than E1 and E2.

Aspect 75. The composition of aspect 74, wherein:
a) the HCV Core-E1-E2 polyprotein of genotype 1 comprises an amino acid sequence having at least 90%, or at least 95%, amino acid sequence identity to an H77 Core-E1-E2 polyprotein;
b) the HCV Core-E1-E2 polyprotein of genotype 2 comprises an amino acid sequence having at least 90%, or at least 95%, amino acid sequence identity to a J6 Core-E1-E2 polyprotein; and
c) the HCV Core-E1-E2 polyprotein of genotype 3 comprises an amino acid sequence having at least 90%, or at least 95%, amino acid sequence identity to an S52 Core-E1-E2 polyprotein.

Aspect 76. The composition of any one of aspects 64-75, wherein the fusion polypeptide comprising two or more heterologous polypeptides comprising a T-cell epitope present in an HCV protein other than E1 and E2 comprises:
a) a TP50C T-cell epitope polypeptide comprising an amino acid sequence having at least 80% amino acid sequence identity to:

GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; Xⱼ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 77. The composition of any one of aspects 64-75, wherein the fusion polypeptide comprising two or more heterologous polypeptides comprising a T-cell epitope present in an HCV protein other than E1 and E2 comprises, in order from N-terminus to C-terminus:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 78. The composition of any one of aspects 1-77, wherein the RNA is a self-amplifying RNA comprising a nucleotide sequence encoding an RNA-dependent RNA polymerase (RdRP).

Aspect 79. The composition of aspect 78, wherein the self-amplifying RNA is a cis-replicon.

Aspect 80. The composition of aspect 78 or aspect 79, wherein the self-amplifying RNA is a recombinant Venezuelan equine encephalitis virus, a Sindbis virus, or a recombinant Semliki Forest virus.

Aspect 81. The composition of any one of aspects 1-80, wherein the composition is in the form of a lipid nanoparticle.

Aspect 82. The composition of any one of aspects 1-80, wherein the composition is in the form of a liposome.

Aspect 83. The composition of any one of aspects 1-80, wherein the composition comprises one or more lipids selected from cationic lipids, neutral lipids, anionic lipids, helper lipids, and stealth lipids.

Aspect 84. The composition of any one of aspects 1-80, wherein the composition comprises an HCV Core polypeptide and wherein the one or more RNA molecules are within a virus-like particle comprising the HCV Core polypeptide.

Aspect 85. A method of inducing an immune response to hepatitis C virus (HCV) in an individual, the method comprising administering to the individual an effective amount of a composition of any one of aspects 1-84.

Aspect 86. The method of aspect 85, comprising:
a) administering a first dose of the composition at a first time; and
b) administering a second dose of the composition at a second time.

Aspect 87. The method of aspect 86, wherein the second time is from a period of time of from about 1 week to about 6 months from the first time.

Aspect 88. The method of any one of aspects 85-87, wherein said administering is intramuscular administration.

Aspect 89. The method of any one of aspects 85-87, wherein said administering is subcutaneous administration.

Aspect 90. The method of any one of aspects 85-89, comprising administering one or more heterologous polypeptides comprising a T-cell epitope present in an HCV protein other than E1 and E2.

Aspect 91. The method of any one of aspect 85-90, comprising administering an HCV E1 polypeptide and/or an HCV E2 polypeptide.

Aspect 92. The method of aspect 86, wherein: a) the first dose comprises one or more RNA molecules comprising a polypeptide-encoding region comprising one or more nucleotide sequences encoding a hepatitis C virus (HCV) E1 polypeptide and/or an HCV E2 polypeptide of a first genotype; and b) the second dose comprises one or more RNA molecules comprising a polypeptide-encoding region comprising one or more nucleotide sequences encoding a hepatitis C virus (HCV) E1 polypeptide and/or an HCV E2 polypeptide of a second genotype.

Aspect 93. The method of aspect 92, wherein: a) the first dose comprises a composition of any one of aspects 64-76; and b) the second dose comprises a composition of any one of aspects 64-76.

Aspect 94. The method of aspect 92, wherein:
a1) the first dose comprises a composition of any one of aspects 64-76, wherein the HCV E1 and/or HCV E2 polypeptide are of genotype 1; and
b1) the second dose comprises a composition of any one of aspects 64-76, wherein the HCV E1 and/or HCV E2 polypeptide are of genotype 3; or
2) the first dose comprises a composition of any one of aspects 64-76, wherein the HCV E1 and/or HCV E2 polypeptide are of genotype 1; and
b2) the second dose comprises a composition of any one of aspects 64-76, wherein the HCV E1 and/or HCV E2 polypeptide are of genotype 4; or
a3) the first dose comprises a composition of any one of aspects 64-76, wherein the HCV E1 and/or HCV E2 polypeptide are of genotype 1; and
b3) the second dose comprises a composition of any one of aspects 64-76, wherein the HCV E1 and/or HCV E2 polypeptide are of genotype 5; or
a4) the first dose comprises a composition of any one of aspects 64-76, wherein the HCV E1 and/or HCV E2 polypeptide are of genotype 1; and
b4) the second dose comprises a composition of any one of aspects 64-76, wherein the HCV E1 and/or HCV E2 polypeptide are of genotype 6; or
a5) the first dose comprises a composition of any one of aspects 64-76, wherein the HCV E1 and/or HCV E2 polypeptide are of genotype 1; and
b5) the second dose comprises a composition of any one of aspects 64-76, wherein the HCV E1 and/or HCV E2 polypeptide are of genotype 2.

Aspect 95. The method of any one of aspect 86-92, comprising administering at least a third dose of the composition at a third time.

### Aspects Set C

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A composition comprising: a) one or more RNA molecules, wherein the one or more RNA molecules comprises: i) at least one 5′ cap structure and/or a 5′ untranslated region (5′ UTR) and/or a 5′ internal ribosome entry site (IRES); ii) a polypeptide-encoding region comprising one or more nucleotide sequences encoding a hepatitis C virus (HCV) E1 polypeptide and/or an HCV E2 polypeptide; and b) one or more of: i) a lipid; ii) a polymer; iii) a peptide; iv) a viral-like particle; and v) a cationic nanoemulsion.

Aspect 2. The composition of aspect 1, wherein the one or more RNA molecules comprises a 5′ cap structure and a 5′ UTR, wherein the 5′UTR is 3′ of the 5′ cap structure.

Aspect 3. The composition of aspect 1 or aspect 2, wherein the one or more RNA molecules comprises a 3′ UTR and/or a 3′ tailing sequence.

Aspect 4. The composition of aspect 3, wherein the 3′ tailing sequence is a poly(adenosine) (poly-A) sequence.

Aspect 5. The composition of any one of aspects 1-4, wherein the one or more RNA molecules include at least one 5′ cap structure, and wherein the at least one 5′ cap structure is selected from the group consisting of Cap0, Cap1, ARCA, inosine, N1-methyl-guanosine, 2′fluoro-guanosine, 7-deaza-guanosine, 8-oxo-guanosine, 2-amino-guanosine, LNA-guanosine, and 2-azido-guanosine.

Aspect 6. The composition of any one of aspects 1-5, wherein the one or more RNA molecules comprises one or more of: a nucleoside base modification, a sugar modification, and a backbone modification.

Aspect 7. The composition any one of aspects 1-6, wherein at least one of the one or more RNA molecules comprises a nucleotide of Formula I:

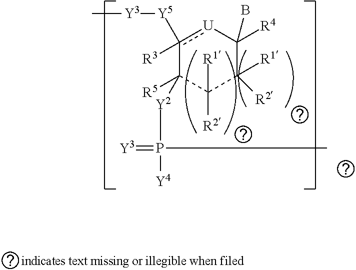

wherein:
U is O, S, N(R^{U})ₙᵤ, or C(R^{U})ₙᵤ, where nu is an integer nu is an integer from 0 to 2 and each R^{U} is, independently, H, halo, or optionally substituted alkyl;
[symbol] is a single or double bond;
[symbol] is a single bond or absent;
each of R^{1′}, R^{2′}, R^{1″}, R^{2″}, R³, R⁴, and R⁵ is, independently, H, halo, hydroxy, thiol, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, or absent; wherein the combination of R³ with one or more of R^{1′}, R^{1″}, R^{2′}, R^{2″}, or R⁵ can join together to form optionally substituted alkylene or optionally substituted heteroalkylene and, taken together with the carbons to which they are attached, provide an optionally substituted heterocyclyl; wherein the combination of R⁵ with one or more of R^{1′}, R^{1″}, R^{2′}, or R^{2″} can join together to form optionally substituted alkylene or optionally substituted heteroalkylene and, taken together with the carbons to which they are attached, provide an optionally substituted heterocyclyl; and wherein the combination of R⁴ and one or more of R^{1′}, R^{1″}, R^{2′}, R^{2″}, R³, or R⁵ can join together to form optionally substituted alkylene or optionally substituted heteroalkylene and, taken together with the carbons to which they are attached, provide an optionally substituted heterocyclyl;
each of Y¹, Y², and Y³, is, independently, O, S, —NR^{N1}—, optionally substituted alkylene, or optionally substituted heteroalkylene, wherein R^{N1} is H, optionally substituted alkyl, optionally substituted alkenyl, optionally substituted alkynyl, optionally substituted aryl, or absent;
each Y⁴ is, independently, H, hydroxy, thiol, optionally substituted alkyl, optionally substituted alkenyl, optionally substituted alkynyl, optionally substituted alkoxy, optionally substituted alkenyloxy, optionally substituted alkynyloxy, optionally substituted thioalkoxy, optionally substituted alkoxyalkoxy, or optionally substituted amino;
each Y⁵ is, independently, O, S, optionally substituted alkylene, or optionally substituted heteroalkylene;
n is an integer from 1 to 100,000; and
B is a nucleobase, wherein the combination of B and R^{1′}, the combination of B and R^{2′}, the combination of B and R^{1″}, or the combination of B and R^{2″} can, taken together with the carbons to which they are attached, optionally form a bicyclic group or wherein the combination of B, R^{1″}, and R³ or the combination of B, R^{2″}, and R³ can optionally form a tricyclic or tetracyclic group.

Aspect 8. The composition of any one of aspects 1-7, wherein the composition comprises a lipid, and wherein the lipid is selected from DLin-DMA, DLin-K-DMA, DLin-KC2-DMA, 98N12-5, C12-200, DLin-MC3-DMA, DODMA, DSDMA, DLenDMA, reLNPs, PLGA, a PEGylated lipid, and mixtures of two or more of the foregoing.

Aspect 9. The composition of any one of aspects 1-7, wherein the composition comprises a polymer.

Aspect 10. The composition of aspect 9, herein the polymer is selected from polyethylene glycol, polyglycolide, polyvinyl alcohol, polyvinyl pyrrolidone, polylactide, poly(lactide-co-glycolide) (PLGA), polycaprolactone, polysorbate, polyethylene oxide, polypropylene oxide, poly(ethylene oxide-co-propylene oxide), poloxamer, poloxamine, poly(oxyethylated) glycerol, poly(oxyethylated) sorbitol, poly(oxyethylated) glucose, polyethyleneimine, polyamidoamine (PAMAM) dendrimer, and block copolymer poly(ethylene glycol)-block-poly(lactic-co-glycolic acid) (PEG-b-PLGA).

Aspect 11. The composition of any one of aspects 1-10, wherein the HCV E1 polypeptide is a soluble HCV E1 polypeptide.

Aspect 12. The composition of any one of aspects 1-10, wherein the HCV E2 polypeptide is a soluble HCV E2 polypeptide.

Aspect 13. The composition of any one of aspects 1-12, wherein the nucleotide sequence encodes an HCV E1 polypeptide and an HCV E2 polypeptide.

Aspect 14. The composition of aspect 13, wherein:
a) the HCV E1 polypeptide is a soluble HCV E1 polypeptide;
b) the HCV E2 polypeptide is a soluble HCV E2 polypeptide; or
c) the HCV E1 polypeptide is a soluble HCV E1 polypeptide and the HCV E2 polypeptide is a soluble HCV E2 polypeptide.

Aspect 15. The composition of any one of aspects 1-14, wherein the polypeptide-encoding region comprises a nucleotide sequence encoding an HCV Core polypeptide.

Aspect 16. The composition of aspect 15, wherein the polypeptide-encoding region comprises a nucleotide sequence encoding an HCV Core-E1-E2 polyprotein.

Aspect 17. The composition of any one of aspects 1-16, wherein the polypeptide-encoding region comprises:
a) one or more nucleotide sequences encoding one or more heterologous polypeptides comprising a T-cell epitope present in an HCV protein other than E1 and E2; or
b) a nucleotide sequence encoding a fusion polypeptide comprising two or more heterologous polypeptides comprising a T-cell epitope present in an HCV protein other than E1 and E2.

Aspect 18. The composition of aspect 17, wherein the RNA comprises:
a) an internal ribosome entry site (IRES) between i) the one or more nucleotide sequences encoding the HCV E1 and/or E2 polypeptides; and ii) the one or more nucleotide sequences encoding the one or more heterologous polypeptides or the nucleotide sequence encoding the fusion polypeptide; or
b) a nucleotide sequence encoding a self-cleaving peptide between i) the one or more nucleotide sequences encoding the HCV E1 and/or E2 polypeptides; and ii) the one or more nucleotide sequences encoding the one or more heterologous polypeptides or the nucleotide sequence encoding the fusion polypeptide.

Aspect 19. The composition of aspect 17 or aspect 18, wherein the one or more heterologous polypeptides is selected from:
i) a TP35-NS3 T-cell epitope polypeptide comprising an amino acid sequence having at least 80% amino acid sequence identity to: KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
ii) a TP50C T-cell epitope polypeptide comprising an amino acid sequence having at least 80% amino acid sequence identity to: GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₂ is S, T, H, or A; X₁₂ is A or G; and X₁₃ is Q or K, wherein the TP50C T-cell epitope polypeptide has a length of from 40 amino acids to 50 amino acids;
iii) a TP23 T-cell epitope polypeptide comprising an amino acid sequence having at least 80% amino acid sequence identity to: DVVVX₁X₂TDALMTGX₃TGDFDSVID (SEQ ID NO:257), wherein X₁ is V or C; X₂ is A or S; and X₃ is F or Y, wherein the TP23 T-cell epitope polypeptide has a length of from 18 amino acids to 23 amino acids;
iv) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids;
v) a TP35-NS4 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀ (SEQ ID NO:288), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; and X₁₀ is V or I, wherein the TP35-NS4 T-cell epitope polypeptide has a length of from 28 amino acids to 35 amino acids;
vi) a TP42 T-cell epitope polypeptide comprising an amino acid sequence having at least 80% amino acid sequence identity to: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:314), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S, wherein n is an integer from 2 to 10, and wherein the TP42 T-cell epitope polypeptide has a length of from 34 amino acids to 52 amino acids;
vii) a TP45 T-cell epitope polypeptide comprising an amino acid sequence having at least 80% amino acid sequence identity to: X₁X₂AVAX₃YRGX₄DVX₅X₆IPX₇X₈GDVVVX₉X₁₀TDALMTGX₁₁TGDFDSVIDX₁₂X₁₃X₁₄(K)n (SEQ ID NO:332), wherein X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₂ is F or Y; X₁₂ is C or K; X₁₃ is N or K; and X₁₄ is V or K, wherein n is an integer from 2 to 10, and wherein the TP45 T-cell epitope polypeptide has a length of from 36 amino acids to 55 amino acids; and
viii) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acids;
ix) a TP33 T-cell epitope polypeptide comprising an amino acid sequence having at least 80% amino acid sequence identity to: HSKKKCDELAX₁X₂LX₃X₄X₅GX₆NAVAYYRGLDVSX₇IP (SEQ ID NO:366), where X₁ is A or S; X₂ is K or A; X₃ is V, S, R, or T; X₄ is A or G; X₅ is L or M; X₆ is I, L, or V; and X₇ is V or I; wherein the TP33 T-cell epitope polypeptide has a length of from 30 amino acids to 36 amino acids;
x) a TP42-2 T-cell epitope polypeptide comprising an amino acid sequence having at least 80% amino acid sequence identity to: KGGRHLIFCHSKKKCDELAX₁X₂LX₃X₄X₅GX₆NAVAYYRGLDVSX₇IP (SEQ ID NO:371), where X₁ is A or S; X₂ is K or A; X₃ is V, S, R, or T; X₄ is A or G; X₅ is L or M; X₆ is I, L, or V; and X₇ is V or I; wherein the TP42-2 T-cell epitope polypeptide has a length of from 38 amino acids to 46 amino acids;
xi) a TP240 T-cell epitope polypeptide comprising an amino acid sequence having at least 80% amino acid sequence identity to:

(SEQ ID NO: 379)
SYQVGYLHAPTGSGKSTKVPAAYAAQGYKVLVLNPSVAATLGFGAYMSK
AHGIDPNIRTGVRTVTTGAPITYSTYGKFLADGGCSGGAYDIIICDECH
SX₁DATTILGIGTVLDQAETAGARLVVLATATPPGSVTVPHPNIEEVAL
GX₂EGEIPFYGKAIPLX₃X₄IKGGRHLIFCHSKKKCDELAAKLRGMGLN
AVAYYRGLDVSVIPTX₅GDVVVVATDALMTGYTGDFDSVIDCNVAVTQT,

where X₁ is T, V, or Q, X₂ is T, N, Q, H, or S, X₃ is E, S, or A, X₄ is V, T, Y, F, or L, and X₅ is S, Q, or T; wherein the TP240 T-cell epitope polypeptide has a length of from 240 amino acids to 245 amino acids;
xii) a TP65 T-cell epitope polypeptide comprising an amino acid sequence having at least 80% amino acid sequence identity to: TPIDTTIMAKNEVFCVDPX₆KGGRKPARLIVYPDLGVRVCEKMALYDVVQKLPQAVMGX₇SYGF QYS (SEQ ID NO:375), where X₆ is E, T, or V, and X₇ is S, A, or P; and wherein the TP65 T-cell epitope polypeptide has a length of from 65 amino acids to 70 amino acids;
xiii) a TP156 T-cell epitope polypeptide comprising an amino acid sequence having at least 80% amino acid sequence identity to the TP156 amino acid sequence depicted in FIG. 32C, where the TP156 T-cell epitope polypeptide has a length of from 150 amino acids to 160 amino acids; and
xiv) a TP465 T-cell epitope polypeptide comprising an amino acid sequence having at least 80% amino acid sequence identity to the TP465 amino acid sequence depicted in any one of FIGS. 32B, 33B, and 34B, where the TP465 T-cell epitope polypeptide has a length of from 460 amino acids to 470 amino acids.

Aspect 20. The composition of any one of aspects 17-19, wherein the polypeptide-encoding region comprises one or more nucleotide sequences encoding two or more heterologous polypeptides comprising a T-cell epitope present in an HCV protein other than E1 and E2.

Aspect 21. The composition of aspect 20, wherein the mRNA comprises:
a) a first internal ribosome entry site (IRES) between i) the one or more nucleotide sequences encoding the HCV E1 and/or E2 polypeptides; and ii) the nucleotide sequence encoding the first of the two or more heterologous polypeptides; and
b) a second IRES between i) the nucleotide sequence encoding the first of the two or more heterologous polypeptides; and ii) the nucleotide sequence encoding the second of the two or more heterologous polypeptides,
wherein the first IRES and the second IRES are the same or different.

Aspect 22. The composition of aspect 21, wherein the mRNA comprises an IRES between any two consecutive nucleotide sequences encoding heterologous polypeptides.

Aspect 23. The composition of aspect 17 or aspect 18, wherein fusion polypeptide comprises from 2 to 10 polypeptides selected from the group consisting of:
i) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids; and
ii) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; Xⱼ is R or K; X₁₁ is S, T, H, or A; X₁₂ is A or G; and X₁₃ is Q or K, wherein the TP50C T-cell epitope polypeptide has a length of from 40 amino acids to 50 amino acids;
iii) a TP23 T-cell epitope polypeptide comprising an amino acid sequence having at least 80% amino acid sequence identity to: DVVVX₁X₂TDALMTGX₃TGDFDSVID (SEQ ID NO:257), wherein X₁ is V or C; X₂ is A or S; and X₃ is F or Y, wherein the TP23 T-cell epitope polypeptide has a length of from 18 amino acids to 23 amino acids;
iv) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids;
v) a TP35-NS4 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀ (SEQ ID NO:288), wherein X₁ is I or V; X₂ is L or I; X₃ is R or K; X₄ is V, I, or T; X₅ is P, Q, or T; X₆ is G, A, or S; X₇ is A or V; X₅ is V or T; X₉ is S or A; and X₁₀ is V or I, wherein the TP35-NS4 T-cell epitope polypeptide has a length of from 28 amino acids to 35 amino acids;
vi) a TP42 T-cell epitope polypeptide comprising an amino acid sequence having at least 80% amino acid sequence identity to: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:314), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S, wherein n is an integer from 2 to 10, and wherein the TP42 T-cell epitope polypeptide has a length of from 34 amino acids to 52 amino acids;
vii) a TP45 T-cell epitope polypeptide comprising an amino acid sequence having at least 80% amino acid sequence identity to: X₁X₂AVAX₃YRGX₄DVX₅X₆IPX₇X₈GDVVVX₉X₁₀TDALMTGX₁₁TGDFDSVIDX₁₂X₁₃X₁₄(K)n (SEQ ID NO:332), wherein X₁ is L or V; X₂ is N or T; X₃ is Y or F; X₄ is L or V; X₅ is S or A; X₆ is V or I; X₇ is T or A; X₈ is S, Q, or T; X₉ is V or C; X₁₀ is A or S; X₁₂ is F or Y; X₁₂ is C or K; X₁₃ is N or K; and X₁₄ is V or K, wherein n is an integer from 2 to 10, and wherein the TP45 T-cell epitope polypeptide has a length of from 36 amino acids to 55 amino acids; and
viii) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acids;
ix) a TP33 T-cell epitope polypeptide comprising an amino acid sequence having at least 80% amino acid sequence identity to: HSKKKCDELAX₁X₂LX₃X₄X₅GX₆NAVAYYRGLDVSX₇IP (SEQ ID NO:366), where X₁ is A or S; X₂ is K or A; X₃ is V, S, R, or T; X₄ is A or G; X₅ is L or M; X₆ is I, L, or V; and X₇ is V or I; wherein the TP33 T-cell epitope polypeptide has a length of from 30 amino acids to 36 amino acids; and
x) a TP42-2 T-cell epitope polypeptide comprising an amino acid sequence having at least 80% amino acid sequence identity to: KGGRHLIFCHSKKKCDELAX₁X₂LX₃X₄X₅GX₆NAVAYYRGLDVSX₇IP (SEQ ID NO:371), where X₁ is A or S; X₂ is K or A; X₃ is V, S, R, or T; X₄ is A or G; X₅ is L or M; X₆ is I, L, or V; and X₇ is V or I; wherein the TP42-2 T-cell epitope polypeptide has a length of from 38 amino acids to 46 amino acids;
xi) a TP240 T-cell epitope polypeptide comprising an amino acid sequence having at least 80% amino acid sequence identity to:

(SEQ ID NO: 379)
SYQVGYLHAPTGSGKSTKVPAAYAAQGYKVLVLNPSVAATLGFGAYMSK
AHGIDPNIRTGVRTVTTGAPITYSTYGKFLADGGCSGGAYDIIICDECH
SX₁DATTILGIGTVLDQAETAGARLVVLATATPPGSVTVPHPNIEEVAL
GX₂EGEIPFYGKAIPLX₃X₄IKGGRHLIFCHSKKKCDELAAKLRGMGLN
AVAYYRGLDVSVIPTX₅GDVVVVATDALMTGYTGDFDSVIDCNVAVTQT,

where X₁ is T, V, or Q, X₂ is T, N, Q, H, or S, X₃ is E, S, or A, X₄ is V, T, Y, F, or L, and X₅ is S, Q, or T; wherein the TP240 T-cell epitope polypeptide has a length of from 240 amino acids to 245 amino acids;
xii) a TP65 T-cell epitope polypeptide comprising an amino acid sequence having at least 80% amino acid sequence identity to: TPIDTTIMAKNEVFCVDPX₆KGGRKPARLIVYPDLGVRVCEKMALYDVVQKLPQAVMGX₇SYGF QYS (SEQ ID NO:375), where X₆ is E, T, or V, and X₇ is S, A, or P; and wherein the TP65 T-cell epitope polypeptide has a length of from 65 amino acids to 70 amino acids;
xiii) a TP156 T-cell epitope polypeptide comprising an amino acid sequence having at least 80% amino acid sequence identity to the TP156 amino acid sequence depicted in FIG. 32C, where the TP156 T-cell epitope polypeptide has a length of from 150 amino acids to 160 amino acids; and
xiv) a TP465 T-cell epitope polypeptide comprising an amino acid sequence having at least 80% amino acid sequence identity to the TP465 amino acid sequence depicted in any one of FIGS. 32B, 33B, and 34B, where the TP465 T-cell epitope polypeptide has a length of from 460 amino acids to 470 amino acids.

Aspect 24. The composition of any one of aspects 1-23, wherein the HCV E1 and/or E2 polypeptide is from HCV genotype 1, optionally genotype 1a.

Aspect 25. The composition of any one of aspects 1-23, wherein the HCV E1 and/or E2 polypeptide is from HCV genotype 2, optionally genotype 2a.

Aspect 26. The composition of any one of aspects 1-23, wherein the HCV E1 and/or E2 polypeptide is from HCV genotype 3, optionally genotype 3a.

Aspect 27. The composition of any one of aspects 1-23, wherein the HCV E1 and/or E2 polypeptide is/are from:
a) HCV genotype 1, 2, and 3;
b) HCV genotype 1a, 2, and 3;
c) HCV genotype 1b, 2, and 3; or
d) HCV genotype 1a, 1b, 2, and 3.

Aspect 28. The composition of any one of aspects 24-27, wherein the polypeptide-encoding region comprises a nucleotide sequence encoding an HCV Core polypeptide, wherein the HCV Core polypeptide is of genotype 1, genotype 2, or genotype 3.

Aspect 29. The composition of any one of aspects 17-28, wherein the one or more RNA molecules comprises a first RNA molecule, a second RNA molecule, and a third RNA molecule, wherein:
a) the polypeptide-encoding region of the first RNA molecule comprises:
   i) a nucleotide sequence encoding HCV E1 and/or an HCV E2 polypeptide of a first genotype;
   ii) a first IRES; and
   iii) one or more nucleotide sequences encoding the one or more heterologous polypeptides or the fusion polypeptide;
b) the polypeptide-encoding region of the second RNA molecule comprises:
   i) a nucleotide sequence encoding HCV E1 and/or an HCV E2 polypeptide of a second genotype;
   ii) a second IRES; and
   iii) one or more nucleotide sequences encoding the one or more heterologous polypeptides or the fusion polypeptide; and
c) the polypeptide-encoding region of the third RNA molecule comprises:
   i) a nucleotide sequence encoding HCV E1 and/or an HCV E2 polypeptide of a third genotype;
   ii) a third IRES; and
   iii) one or more nucleotide sequences encoding the one or more heterologous polypeptides or the fusion polypeptide;
   wherein the first IRES, the second IRES, and the third IRES are the same or different.

Aspect 30. The composition of aspect 29, wherein:
a) the HCV E1 polypeptide encoded by the first RNA molecule is a soluble HCV E1 polypeptide and/or the HCV E2 polypeptide encoded by the first RNA molecule is a soluble HCV E2 polypeptide; and/or
b) the HCV E1 polypeptide encoded by the second RNA molecule is a soluble HCV E1 polypeptide and/or the HCV E2 polypeptide encoded by the second RNA molecule is a soluble HCV E2 polypeptide; and/or
c) the HCV E1 polypeptide encoded by the third RNA molecule is a soluble HCV E1 polypeptide and/or the HCV E2 polypeptide encoded by the third RNA molecule is a soluble HCV E2 polypeptide.

Aspect 31. The composition of aspect 29 or aspect 30, wherein the polypeptide-encoding region of one or more of the first RNA molecule, the second RNA molecule, and the third RNA molecule comprises a nucleotide sequence encoding an HCV Core polypeptide.

Aspect 32. The composition of aspect 29 or aspect 30, wherein:
a) the polypeptide-encoding region of the first RNA molecule comprises:
   i) a nucleotide sequence encoding HCV E1 and HCV E2 polypeptides of genotype 1;
   ii) a first IRES; and
   iii) a nucleotide sequence encoding the fusion polypeptide;
b) the polypeptide-encoding region of the second RNA molecule comprises:
   i) a nucleotide sequence encoding HCV E1 and HCV E2 polypeptides of genotype 2;
   ii) a second IRES; and
   iii) a nucleotide sequence encoding the fusion polypeptide; and
c) the polypeptide-encoding region of the third RNA molecule comprises:
   i) a nucleotide sequence encoding HCV E1 and HCV E2 polypeptides of genotype 3;
   ii) a third IRES; and
   iii) a nucleotide sequence encoding the fusion polypeptide;
   wherein the first IRES, the second IRES, and the third IRES are the same or different.

Aspect 33. The composition of aspect 32, wherein:
a) the HCV E1 polypeptide of genotype 1 comprises an amino acid sequence having at least 90%, or at least 95%, amino acid sequence identity to an H77 E1 polypeptide, and the HCV E2 polypeptide of genotype 1 comprises an amino acid sequence having at least 90%, or at least 95%, amino acid sequence identity to an H77 E2 polypeptide;
b) the HCV E1 polypeptide of genotype 2 comprises an amino acid sequence having at least 90%, or at least 95%, amino acid sequence identity to a J6 E1 polypeptide, and the HCV E2 polypeptide of genotype 2 comprises an amino acid sequence having at least 90%, or at least 95%, amino acid sequence identity to a J6 E2 polypeptide; and
c) the HCV E1 polypeptide of genotype 3 comprises an amino acid sequence having at least 90%, or at least 95%, amino acid sequence identity to an S52 E1 polypeptide, and the HCV E2 polypeptide of genotype 3 comprises an amino acid sequence having at least 90%, or at least 95%, amino acid sequence identity to an S52 E2 polypeptide.

Aspect 34. The composition of aspect 32 or aspect 33, wherein the polypeptide-encoding region of one or more of the first RNA molecule, the second RNA molecule, and the third RNA molecule comprises a nucleotide sequence encoding an HCV Core polypeptide.

Aspect 35. The composition of aspect 34, wherein:
a) the polypeptide-encoding region of the first RNA molecule comprises:
   i) a nucleotide sequence encoding an HCV Core-E1-E2 polypeptide of genotype 1;
   ii) a first IRES; and
   iii) a nucleotide sequence encoding the fusion polypeptide;
b) the polypeptide-encoding region of the second RNA molecule comprises:
   i) a nucleotide sequence encoding an HCV Core-E1-E2 polypeptide of genotype 2;
   ii) a second IRES; and
   iii) a nucleotide sequence encoding the fusion polypeptide; and
c) the polypeptide-encoding region of the third RNA molecule comprises:
   i) a nucleotide sequence encoding an HCV Core-E1-E2 polypeptide of genotype 3;
   ii) a third IRES; and
   iii) a nucleotide sequence encoding the fusion polypeptide;
   wherein the first IRES, the second IRES, and the third IRES are the same or different.

Aspect 36. The composition of aspect 35, wherein:
a) the HCV E1 polypeptide of genotype 1 comprises an amino acid sequence having at least 90%, or at least 95%, amino acid sequence identity to an H77 E1 polypeptide, and the HCV E2 polypeptide of genotype 1 comprises an amino acid sequence having at least 90%, or at least 95%, amino acid sequence identity to an H77 E2 polypeptide;
b) the HCV E1 polypeptide of genotype 2 comprises an amino acid sequence having at least 95% amino acid sequence identity to a J6 E1 polypeptide, and the HCV E2 polypeptide of genotype 2 comprises an amino acid sequence having at least 90%, or at least 95%, amino acid sequence identity to a J6 E2 polypeptide; and
c) the HCV E1 polypeptide of genotype 3 comprises an amino acid sequence having at least 90%, or at least 95%, amino acid sequence identity to an S52 E1 polypeptide, and the HCV E2 polypeptide of genotype 3 comprises an amino acid sequence having at least 90%, or at least 95%, amino acid sequence identity to an S52 E2 polypeptide.

Aspect 37. The composition of any one of aspects 17 to 28, wherein:
a) the polypeptide-encoding region of the first RNA molecule comprises a nucleotide sequence encoding a fusion polypeptide comprising:
   i) HCV E1 and HCV2 E2 polypeptides of genotype 1; and
   ii) the fusion polypeptide comprising two or more heterologous polypeptides comprising a T-cell epitope present in an HCV protein other than E1 and E2;
b) the polypeptide-encoding region of the second RNA molecule comprises a nucleotide sequence encoding a fusion polypeptide comprising:
   i) HCV E1 and HCV2 E2 polypeptides of genotype 2; and
   ii) the fusion polypeptide comprising two or more heterologous polypeptides comprising a T-cell epitope present in an HCV protein other than E1 and E2; and
c) the polypeptide-encoding region of the third RNA molecule comprises a nucleotide sequence encoding a fusion polypeptide comprising:
   i) HCV E1 and HCV2 E2 polypeptides of genotype 3; and
   ii) the fusion polypeptide comprising two or more heterologous polypeptides comprising a T-cell epitope present in an HCV protein other than E1 and E2.

Aspect 38. The composition of aspect 37, wherein:
a) the HCV E1 polypeptide of genotype 1 comprises an amino acid sequence having at least 90%, or at least 95%, amino acid sequence identity to an H77 E1 polypeptide, and the HCV E2 polypeptide of genotype 1 comprises an amino acid sequence having at least 90%, or at least 95%, amino acid sequence identity to an H77 E2 polypeptide;
b) the HCV E1 polypeptide of genotype 2 comprises an amino acid sequence having at least 90%, or at least 95%, amino acid sequence identity to a J6 E1 polypeptide, and the HCV E2 polypeptide of genotype 2 comprises an amino acid sequence having at least 90%, or at least 95%, amino acid sequence identity to a J6 E2 polypeptide; and
c) the HCV E1 polypeptide of genotype 3 comprises an amino acid sequence having at least 90%, or at least 95%, amino acid sequence identity to an S52 E1 polypeptide, and the HCV E2 polypeptide of genotype 3 comprises an amino acid sequence having at least 90%, or at least 95%, amino acid sequence identity to an S52 E2 polypeptide.

Aspect 39. The composition of aspect 37, wherein:
a) the HCV E1 polypeptide encoded by the first RNA molecule is a soluble HCV E1 polypeptide and/or the HCV E2 polypeptide encoded by the first RNA molecule is a soluble HCV E2 polypeptide; and/or
b) the HCV E1 polypeptide encoded by the second RNA molecule is a soluble HCV E1 polypeptide and/or the HCV E2 polypeptide encoded by the second RNA molecule is a soluble HCV E2 polypeptide; and/or
c) the HCV E1 polypeptide encoded by the third RNA molecule is a soluble HCV E1 polypeptide and/or the HCV E2 polypeptide encoded by the third RNA molecule is a soluble HCV E2 polypeptide.

Aspect 40. The composition of aspect 39, wherein:
a) the soluble HCV E1 polypeptide of genotype 1 comprises an amino acid sequence having at least 90%, or at least 95%, amino acid sequence identity to a soluble H77 E1 polypeptide, and the soluble HCV E2 polypeptide of genotype 1 comprises an amino acid sequence having at least 90%, or at least 95%, amino acid sequence identity to a soluble H77 E2 polypeptide;
b) the soluble HCV E1 polypeptide of genotype 2 comprises an amino acid sequence having at least 90%, or at least 95%, amino acid sequence identity to a soluble J6 E1 polypeptide, and the soluble HCV E2 polypeptide of genotype 2 comprises an amino acid sequence having at least 90%, or at least 95%, amino acid sequence identity to a soluble J6 E2 polypeptide; and
c) the soluble HCV E1 polypeptide of genotype 3 comprises an amino acid sequence having at least 90%, or at least 95%, amino acid sequence identity to a soluble S52 E1 polypeptide, and the soluble HCV E2 polypeptide of genotype 3 comprises an amino acid sequence having at least 90%, or at least 95%, amino acid sequence identity to a soluble S52 E2 polypeptide.

Aspect 41. The composition of any one of aspects 37-40, wherein the polypeptide-encoding region of one or more of the first RNA molecule, the second RNA molecule, and the third RNA molecule comprises a nucleotide sequence encoding an HCV Core polypeptide.

Aspect 42. The composition of aspect 41, wherein:
a) the polypeptide-encoding region of the first RNA molecule comprises a nucleotide sequence encoding a fusion polypeptide comprising:
   i) an HCV Core-E1-E2 polyprotein of genotype 1; and
   ii) the fusion polypeptide comprising two or more heterologous polypeptides comprising a T-cell epitope present in an HCV protein other than E1 and E2;
b) the polypeptide-encoding region of the second RNA molecule comprises a nucleotide sequence encoding a fusion polypeptide comprising:
   i) an HCV Core-E1-E2 polyprotein of genotype 2; and
   ii) the fusion polypeptide comprising two or more heterologous polypeptides comprising a T-cell epitope present in an HCV protein other than E1 and E2; and
c) the polypeptide-encoding region of the third RNA molecule comprises a nucleotide sequence encoding a fusion polypeptide comprising:
   i) an HCV Core-E1-E2 polyprotein of genotype 3; and
   ii) the fusion polypeptide comprising two or more heterologous polypeptides comprising a T-cell epitope present in an HCV protein other than E1 and E2.

Aspect 43. The composition of aspect 42, wherein:
a) the HCV Core-E1-E2 polyprotein of genotype 1 comprises an amino acid sequence having at least 90%, or at least 95%, amino acid sequence identity to an H77 Core-E1-E2 polyprotein;
b) the HCV Core-E1-E2 polyprotein of genotype 2 comprises an amino acid sequence having at least 90%, or at least 95%, amino acid sequence identity to a J6 Core-E1-E2 polyprotein; and
c) the HCV Core-E1-E2 polyprotein of genotype 3 comprises an amino acid sequence having at least 90%, or at least 95%, amino acid sequence identity to an S52 Core-E1-E2 polyprotein.

Aspect 44. The composition of aspect 41, wherein:
a) the polypeptide-encoding region of the first RNA molecule comprises a nucleotide sequence encoding a fusion polypeptide comprising:
   i) an HCV Core-E1-E2 polyprotein of genotype 1; and
   ii) the fusion polypeptide comprising two or more heterologous polypeptides comprising a T-cell epitope present in an HCV protein other than E1 and E2;
b) the polypeptide-encoding region of the second RNA molecule comprises a nucleotide sequence encoding a fusion polypeptide comprising:
   i) an HCV Core-E1-E2 polyprotein of genotype 3; and
   ii) the fusion polypeptide comprising two or more heterologous polypeptides comprising a T-cell epitope present in an HCV protein other than E1 and E2; and
c) the polypeptide-encoding region of the third RNA molecule comprises a nucleotide sequence encoding a fusion polypeptide comprising:
   i) an HCV Core-E1-E2 polyprotein of genotype 4, genotype 5, or genotype 6; and
   ii) the fusion polypeptide comprising two or more heterologous polypeptides comprising a T-cell epitope present in an HCV protein other than E1 and E2.

Aspect 45. The composition of any one of aspects 23-44, wherein the fusion polypeptide comprising two or more heterologous polypeptides comprising a T-cell epitope present in an HCV protein other than E1 and E2 comprises:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; Xⱼ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 46. The composition of any one of aspects 23-45, wherein the fusion polypeptide comprising two or more heterologous polypeptides comprising a T-cell epitope present in an HCV protein other than E1 and E2 comprises, in order from N-terminus to C-terminus:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to:
X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 47. The composition of any one of aspects 17-28, wherein the one or more RNA molecules comprises a first RNA molecule and a second RNA molecule, wherein:
a) the polypeptide-encoding region of the first RNA molecule comprises:
   i) a nucleotide sequence encoding an HCV E1 and/or an HCV E2 polypeptide of a first genotype;
   ii) a first IRES; and
   iii) one or more nucleotide sequences encoding the one or more heterologous polypeptides or the fusion polypeptide; and
b) the polypeptide-encoding region of the second RNA molecule comprises:
   i) a nucleotide sequence encoding an HCV E1 and/or an HCV E2 polypeptide of a second genotype;
   ii) a second IRES; and
   iii) one or more nucleotide sequences encoding the one or more heterologous polypeptides or the fusion polypeptide;
wherein the first IRES and the second IRES are the same or different.

Aspect 48. The composition of aspect 47, wherein:
a) the HCV E1 polypeptide encoded by the first RNA molecule is a soluble HCV E1 polypeptide and/or the HCV E2 polypeptide encoded by the first RNA molecule is a soluble HCV E2 polypeptide; and/or
b) the HCV E1 polypeptide encoded by the second RNA molecule is a soluble HCV E1 polypeptide and/or the HCV E2 polypeptide encoded by the second RNA molecule is a soluble HCV E2 polypeptide.

Aspect 49. The composition of aspect 47 or 48, wherein the polypeptide-encoding region of one or both of the first RNA molecule and the second RNA molecule comprises a nucleotide sequence encoding an HCV Core polypeptide.

Aspect 50. The composition of aspect 47 or 48, wherein:
a1) the polypeptide-encoding region of the first RNA molecule comprises:
   i) a nucleotide sequence encoding HCV E1 and HCV E2 polypeptides of genotype 1;
   ii) a first IRES; and
   iii) a nucleotide sequence encoding the fusion polypeptide; and
b1) the polypeptide-encoding region of the second RNA molecule comprises:
   i) a nucleotide sequence encoding HCV E1 and HCV E2 polypeptides of genotype 3;
   ii) a second IRES; and
   iii) a nucleotide sequence encoding the fusion polypeptide; or
a2) the polypeptide-encoding region of the first RNA molecule comprises:
   i) a nucleotide sequence encoding HCV E1 and HCV E2 polypeptides of genotype 1;
   ii) a first IRES; and
   iii) a nucleotide sequence encoding the fusion polypeptide; and
b2) the polypeptide-encoding region of the second RNA molecule comprises:
   i) a nucleotide sequence encoding HCV E1 and HCV E2 polypeptides of genotype 2;
   ii) a second IRES; and
   iii) a nucleotide sequence encoding the fusion polypeptide; or
a3) the polypeptide-encoding region of the first RNA molecule comprises:
   i) a nucleotide sequence encoding HCV E1 and HCV E2 polypeptides of genotype 2;
   ii) a first IRES; and
   iii) a nucleotide sequence encoding the fusion polypeptide; and
b3) the polypeptide-encoding region of the second RNA molecule comprises:
   i) a nucleotide sequence encoding HCV E1 and HCV E2 polypeptides of genotype 3;
   ii) a second IRES; and
   iii) a nucleotide sequence encoding the fusion polypeptide;
wherein the first IRES and the second IRES are the same or different; or
a4) the first RNA molecule comprises.
   i) a nucleotide sequence encoding HCV E1 and HCV E2 polypeptides of a first genotype, optionally wherein the first genotype is genotype 1a, optionally wherein the HCV E1 and E2 polypeptides comprise amino acid sequences having at least 80% amino acid sequence identity to the E1 and E2 amino acid sequences depicted in FIG. 32A;
   ii) a first IRES;
   iii) a nucleotide sequence encoding a TP465 polypeptide comprising an amino acid sequence having at least 80% amino acid sequence identity to the TP465 polypeptide amino acid sequence depicted in FIG. 32B, FIG. 33B, or FIG. 34B;
   iv) a second IRES; and
   v) a nucleotide sequence encoding a TP156 polypeptide comprising an amino acid sequence having at least 80% amino acid sequence identity to the TP156 polypeptide amino acid sequence depicted in FIG. 32C; and
b4) the second RNA molecule comprises:
   i) a nucleotide sequence encoding HCV E1 and/or an HCV E2 polypeptide of a second genotype, optionally wherein the second genotype is genotype 2a, optionally wherein the HCV E1 and E2 polypeptides comprise amino acid sequences having at least 80% amino acid sequence identity to the E1 and E2 amino acid sequences depicted in FIG. 34A;
   ii) a first IRES;
   iii) a nucleotide sequence encoding a TP465 polypeptide comprising an amino acid sequence having at least 80% amino acid sequence identity to the TP465 polypeptide amino acid sequence depicted in FIG. 32B, FIG. 33B, or FIG. 34B;
   iv) a second IRES; and
   v) a nucleotide sequence encoding a TP65 polypeptide comprising an amino acid sequence having at least 80% amino acid sequence identity to the TP65 polypeptide amino acid sequence depicted in FIG. 34C; or
a5) the first RNA molecule comprises.
   i) a nucleotide sequence encoding HCV E1 and HCV E2 polypeptides of a first genotype, optionally wherein the first genotype is genotype 1a, optionally wherein the HCV E1 and E2 polypeptides comprise amino acid sequences having at least 80% amino acid sequence identity to the E1 and E2 amino acid sequences depicted in FIG. 32A;
   ii) a first IRES;
   iii) a nucleotide sequence encoding a TP465 polypeptide comprising an amino acid sequence having at least 80% amino acid sequence identity to the TP465 polypeptide amino acid sequence depicted in FIG. 32B, FIG. 33B, or FIG. 34B;
   iv) a second IRES; and
   v) a nucleotide sequence encoding a TP156 polypeptide comprising an amino acid sequence having at least 80% amino acid sequence identity to the TP156 polypeptide amino acid sequence depicted in FIG. 32C; and
b5) the second RNA molecule comprises:
   i) a nucleotide sequence encoding HCV E1 and/or an HCV E2 polypeptide of a second genotype, optionally wherein the second genotype is genotype 3a, optionally wherein the HCV E1 and E2 polypeptides comprise amino acid sequences having at least 80% amino acid sequence identity to the E1 and E2 amino acid sequences depicted in FIG. 33A;
   ii) a first IRES;
   iii) a nucleotide sequence encoding a TP465 polypeptide comprising an amino acid sequence having at least 80% amino acid sequence identity to the TP465 polypeptide amino acid sequence depicted in FIG. 32B, FIG. 33B, or FIG. 34B;
   iv) a second IRES; and
   v) a nucleotide sequence encoding a TP48 polypeptide comprising an amino acid sequence having at least 80% amino acid sequence identity to the TP48 polypeptide amino acid sequence depicted in FIG. 33C; or
a6) the first RNA molecule comprises:
   i) a nucleotide sequence encoding HCV E1 and/or an HCV E2 polypeptide of a first genotype, optionally wherein the first genotype is genotype 3a, optionally wherein the HCV E1 and E2 polypeptides comprise amino acid sequences having at least 80% amino acid sequence identity to the E1 and E2 amino acid sequences depicted in FIG. 33A;
   ii) a first IRES;
   iii) a nucleotide sequence encoding a TP465 polypeptide comprising an amino acid sequence having at least 80% amino acid sequence identity to the TP465 polypeptide amino acid sequence depicted in FIG. 32B, FIG. 33B, or FIG. 34B;
   iv) a second IRES; and
   v) a nucleotide sequence encoding a TP48 polypeptide comprising an amino acid sequence having at least 80% amino acid sequence identity to the TP48 polypeptide amino acid sequence depicted in FIG. 33C; and
b6) the second RNA molecule comprises:
   i) a nucleotide sequence encoding HCV E1 and/or an HCV E2 polypeptide of a second genotype, optionally wherein the second genotype is genotype 2a, optionally wherein the HCV E1 and E2 polypeptides comprise amino acid sequences having at least 80% amino acid sequence identity to the E1 and E2 amino acid sequences depicted in FIG. 34A;
   ii) a first IRES;
   iii) a nucleotide sequence encoding a TP465 polypeptide comprising an amino acid sequence having at least 80% amino acid sequence identity to the TP465 polypeptide amino acid sequence depicted in FIG. 32B, FIG. 33B, or FIG. 34B;
   iv) a second IRES; and
   v) a nucleotide sequence encoding a TP65 polypeptide comprising an amino acid sequence having at least 80% amino acid sequence identity to the TP65 polypeptide amino acid sequence depicted in FIG. 34C.

Aspect 51. The composition of aspect 50, wherein:
a) the HCV E1 polypeptide of genotype 1 comprises an amino acid sequence having at least 90%, or at least 95%, amino acid sequence identity to an H77 E1 polypeptide, and the HCV E2 polypeptide of genotype 1 comprises an amino acid sequence having at least 90%, or at least 95%, amino acid sequence identity to an H77 E2 polypeptide;
b) the HCV E1 polypeptide of genotype 2 comprises an amino acid sequence having at least 90%, or at least 95%, amino acid sequence identity to a J6 E1 polypeptide, and the HCV E2 polypeptide of genotype 2 comprises an amino acid sequence having at least 90%, or at least 95%, amino acid sequence identity to a J6 E2 polypeptide; and
c) the HCV E1 polypeptide of genotype 3 comprises an amino acid sequence having at least 90%, or at least 95%, amino acid sequence identity to an S52 E1 polypeptide, and the HCV E2 polypeptide of genotype 3 comprises an amino acid sequence having at least 90%, or at least 95%, amino acid sequence identity to an S52 E2 polypeptide.

Aspect 52. The composition of aspect 50 or aspect 51, wherein the polypeptide-encoding region of one or both of the first RNA molecule and the second RNA molecule comprises a nucleotide sequence encoding an HCV Core polypeptide. xxx

Aspect 53. The composition of aspect 52, wherein:
a1) the polypeptide-encoding region of the first RNA molecule comprises:
   i) a nucleotide sequence encoding an HCV Core-E1-E2 polyprotein of genotype 1;
   ii) a first IRES; and
   iii) a nucleotide sequence encoding the fusion polypeptide;
b1) the polypeptide-encoding region of the second RNA molecule comprises:
   i) a nucleotide sequence encoding an HCV Core-E1-E2 polyprotein of genotype 3;
   ii) a second IRES; and
   iii) a nucleotide sequence encoding the fusion polypeptide; or
a2) the polypeptide-encoding region of the first RNA molecule comprises:
   i) a nucleotide sequence encoding an HCV Core-E1-E2 polyprotein of genotype 1;
   ii) a first IRES; and
   iii) a nucleotide sequence encoding the fusion polypeptide;
b2) the polypeptide-encoding region of the second RNA molecule comprises:
   i) a nucleotide sequence encoding an HCV Core-E1-E2 polyprotein of genotype 2;
   ii) a second IRES; and
   iii) a nucleotide sequence encoding the fusion polypeptide; or
a3) the polypeptide-encoding region of the first RNA molecule comprises:
   i) a nucleotide sequence encoding an HCV Core-E1-E2 polyprotein of genotype 2;
   ii) a first IRES; and
   iii) a nucleotide sequence encoding the fusion polypeptide;
b3) the polypeptide-encoding region of the second RNA molecule comprises:
   i) a nucleotide sequence encoding an HCV Core-E1-E2 polyprotein of genotype 3;
   ii) a second IRES; and
   iii) a nucleotide sequence encoding the fusion polypeptide;
wherein the first IRES and the second IRES are the same or different.

Aspect 54. The composition of aspect 53, wherein:
a) the HCV Core-E1-E2 polyprotein of genotype 1 comprises an amino acid sequence having at least 90%, or at least 95%, amino acid sequence identity to an H77 HCV Core-E1-E2 polyprotein;
b) the HCV Core-E1-E2 polyprotein of genotype 2 comprises an amino acid sequence having at least 90%, or at least 95%, amino acid sequence identity to a J6 HCV Core-E1-E2 polyprotein; and
c) the HCV Core-E1-E2 polyprotein of genotype 3 comprises an amino acid sequence at least 90%, or at least 95%, amino acid sequence identity to an S52 HCV Core-E1-E2 polyprotein.

Aspect 55. The composition of any one of aspects 17 to 28, wherein the composition comprises a first RNA molecule and a second RNA molecule, and wherein:
a1) the polypeptide-encoding region of the first RNA molecule comprises a nucleotide sequence encoding a fusion polypeptide comprising:
   i) HCV E1 and HCV2 E2 polypeptides of genotype 1; and
   ii) the fusion polypeptide comprising two or more heterologous polypeptides comprising a T-cell epitope present in an HCV protein other than E1 and E2;
b1) the polypeptide-encoding region of the second RNA molecule comprises a nucleotide sequence encoding a fusion polypeptide comprising:
   i) HCV E1 and HCV2 E2 polypeptides of genotype 3; and
   ii) the fusion polypeptide comprising two or more heterologous polypeptides comprising a T-cell epitope present in an HCV protein other than E1 and E2; or
a2) the polypeptide-encoding region of the first RNA molecule comprises a nucleotide sequence encoding a fusion polypeptide comprising:
   i) HCV E1 and HCV2 E2 polypeptides of genotype 1; and
   ii) the fusion polypeptide comprising two or more heterologous polypeptides comprising a T-cell epitope present in an HCV protein other than E1 and E2;
b2) the polypeptide-encoding region of the second RNA molecule comprises a nucleotide sequence encoding a fusion polypeptide comprising:
   i) HCV E1 and HCV2 E2 polypeptides of genotype 2; and
   ii) the fusion polypeptide comprising two or more heterologous polypeptides comprising a T-cell epitope present in an HCV protein other than E1 and E2; or
a3) the polypeptide-encoding region of the first RNA molecule comprises a nucleotide sequence encoding a fusion polypeptide comprising:
   i) HCV E1 and HCV2 E2 polypeptides of genotype 2; and
   ii) the fusion polypeptide comprising two or more heterologous polypeptides comprising a T-cell epitope present in an HCV protein other than E1 and E2;
b3) the polypeptide-encoding region of the second RNA molecule comprises a nucleotide sequence encoding a fusion polypeptide comprising:
   i) HCV E1 and HCV2 E2 polypeptides of genotype 3; and
   ii) the fusion polypeptide comprising two or more heterologous polypeptides comprising a T-cell epitope present in an HCV protein other than E1 and E2.

Aspect 56. The composition of aspect 55, wherein:
a) the HCV E1 polypeptide of genotype 1 comprises an amino acid sequence having at least 90%, or at least 95%, amino acid sequence identity to an H77 E1 polypeptide, and the HCV E2 polypeptide of genotype 1 comprises an amino acid sequence having at least 90%, or at least 95%, amino acid sequence identity to an H77 E2 polypeptide;
b) the HCV E1 polypeptide of genotype 2 comprises an amino acid sequence having at least 90%, or at least 95%, amino acid sequence identity to a J6 E1 polypeptide, and the HCV E2 polypeptide of genotype 2 comprises an amino acid sequence having at least 90%, or at least 95%, amino acid sequence identity to a J6 E2 polypeptide; and
c) the HCV E1 polypeptide of genotype 3 comprises an amino acid sequence having at least 90%, or at least 95%, amino acid sequence identity to an S52 E1 polypeptide, and the HCV E2 polypeptide of genotype 3 comprises an amino acid sequence having at least 90%, or at least 95%, amino acid sequence identity to an S52 E2 polypeptide.

Aspect 57. The composition of aspect 55, wherein:
a) the HCV E1 polypeptide encoded by the first RNA molecule is a soluble HCV E1 polypeptide and/or the HCV E2 polypeptide encoded by the first RNA molecule is a soluble HCV E2 polypeptide; and/or
b) the HCV E1 polypeptide encoded by the second RNA molecule is a soluble HCV E1 polypeptide and/or the HCV E2 polypeptide encoded by the second RNA molecule is a soluble HCV E2 polypeptide.

Aspect 58. The composition of aspect 57, wherein:
a) the soluble HCV E1 polypeptide of genotype 1 comprises an amino acid sequence having at least 90%, or at least 95%, amino acid sequence identity to a soluble H77 E1 polypeptide, and the soluble HCV E2 polypeptide of genotype 1 comprises an amino acid sequence having at least 90%, or at least 95%, amino acid sequence identity to a soluble H77 E2 polypeptide;
b) the soluble HCV E1 polypeptide of genotype 2 comprises an amino acid sequence having at least 90%, or at least 95%, amino acid sequence identity to a soluble J6 E1 polypeptide, and the soluble HCV E2 polypeptide of genotype 2 comprises an amino acid sequence having at least 90%, or at least 95%, amino acid sequence identity to a soluble J6 E2 polypeptide; and
c) the soluble HCV E1 polypeptide of genotype 3 comprises an amino acid sequence having at least 90%, or at least 95%, amino acid sequence identity to a soluble S52 E1 polypeptide, and the soluble HCV E2 polypeptide of genotype 3 comprises an amino acid sequence having at least 90%, or at least 95%, amino acid sequence identity to a soluble S52 E2 polypeptide.

Aspect 59. The composition of any one of aspects 55-58, wherein the polypeptide-encoding region of one or both of the first RNA molecule and the second RNA molecule comprises a nucleotide sequence encoding an HCV Core polypeptide.

Aspect 60. The composition of aspect 59, wherein:
a1) the polypeptide-encoding region of the first RNA molecule comprises a nucleotide sequence encoding a fusion polypeptide comprising:
   i) an HCV Core-E1-E2 polyprotein of genotype 1; and
   ii) the fusion polypeptide comprising two or more heterologous polypeptides comprising a T-cell epitope present in an HCV protein other than E1 and E2;
b1) the polypeptide-encoding region of the first RNA molecule comprises a nucleotide sequence encoding a fusion polypeptide comprising:
   i) an HCV Core-E1-E2 polyprotein of genotype 3; and
   ii) the fusion polypeptide comprising two or more heterologous polypeptides comprising a T-cell epitope present in an HCV protein other than E1 and E2; or
a2) the polypeptide-encoding region of the first RNA molecule comprises a nucleotide sequence encoding a fusion polypeptide comprising:
   i) an HCV Core-E1-E2 polyprotein of genotype 1; and
   ii) the fusion polypeptide comprising two or more heterologous polypeptides comprising a T-cell epitope present in an HCV protein other than E1 and E2;
b2) the polypeptide-encoding region of the first RNA molecule comprises a nucleotide sequence encoding a fusion polypeptide comprising:
   i) an HCV Core-E1-E2 polyprotein of genotype 2; and
   ii) the fusion polypeptide comprising two or more heterologous polypeptides comprising a T-cell epitope present in an HCV protein other than E1 and E2; or
a3) the polypeptide-encoding region of the first RNA molecule comprises a nucleotide sequence encoding a fusion polypeptide comprising:
   i) an HCV Core-E1-E2 polyprotein of genotype 2; and
   ii) the fusion polypeptide comprising two or more heterologous polypeptides comprising a T-cell epitope present in an HCV protein other than E1 and E2;
b3) the polypeptide-encoding region of the first RNA molecule comprises a nucleotide sequence encoding a fusion polypeptide comprising:
   i) an HCV Core-E1-E2 polyprotein of genotype 3; and
   ii) the fusion polypeptide comprising two or more heterologous polypeptides comprising a T-cell epitope present in an HCV protein other than E1 and E2.

Aspect 61. The composition of aspect 60, wherein:
a) the HCV Core-E1-E2 polyprotein of genotype 1 comprises an amino acid sequence having at least 90%, or at least 95%, amino acid sequence identity to an H77 Core-E1-E2 polyprotein;
b) the HCV Core-E1-E2 polyprotein of genotype 2 comprises an amino acid sequence having at least 90%, or at least 95%, amino acid sequence identity to a J6 Core-E1-E2 polyprotein; and
c) the HCV Core-E1-E2 polyprotein of genotype 3 comprises an amino acid sequence having at least 90%, or at least 95%, amino acid sequence identity to an S52 Core-E1-E2 polyprotein.

Aspect 62. The composition of any one of aspects 47-61, wherein the fusion polypeptide comprising two or more heterologous polypeptides comprising a T-cell epitope present in an HCV protein other than E1 and E2 comprises:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; Xⱼ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 63. The composition of any one of aspects 47-61, wherein the fusion polypeptide comprising two or more heterologous polypeptides comprising a T-cell epitope present in an HCV protein other than E1 and E2 comprises, in order from N-terminus to C-terminus:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; Xⱼ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 64. The composition of any one of aspects 17-28, wherein the composition comprises a single type of RNA molecule, wherein:
a) the polypeptide-encoding region of the RNA molecule comprises:
   i) a nucleotide sequence encoding an HCV E1 and/or an HCV E2 polypeptide of genotype 1;
   ii) a first IRES; and
   iii) one or more nucleotide sequences encoding the one or more heterologous polypeptides or the fusion polypeptide; or
b) the polypeptide-encoding region of the RNA molecule comprises:
   i) a nucleotide sequence encoding an HCV E1 and/or an HCV E2 polypeptide of genotype 2;
   ii) a first IRES; and
   iii) one or more nucleotide sequences encoding the one or more heterologous polypeptides or the fusion polypeptide; or
c) the polypeptide-encoding region of the RNA molecule comprises:
   i) a nucleotide sequence encoding an HCV E1 and/or an HCV E2 polypeptide of genotype 3;
   ii) a first IRES; and
   iii) one or more nucleotide sequences encoding the one or more heterologous polypeptides or the fusion polypeptide; or
d) the polypeptide-encoding region of the RNA molecule comprises:
   i) a nucleotide sequence encoding an HCV E1 and/or an HCV E2 polypeptide of genotype 4;
   ii) a first IRES; and
   iii) one or more nucleotide sequences encoding the one or more heterologous polypeptides or the fusion polypeptide; or
e) the polypeptide-encoding region of the RNA molecule comprises:
   i) a nucleotide sequence encoding an HCV E1 and/or an HCV E2 polypeptide of genotype 5;
   ii) a first IRES; and
   iii) one or more nucleotide sequences encoding the one or more heterologous polypeptides or the fusion polypeptide; or
f) the polypeptide-encoding region of the RNA molecule comprises:
   i) a nucleotide sequence encoding an HCV E1 and/or an HCV E2 polypeptide of genotype 6;
   ii) a first IRES; and
   iii) one or more nucleotide sequences encoding the one or more heterologous polypeptides or the fusion polypeptide.

Aspect 65. The composition of aspect 64, wherein:
a) the HCV E1 polypeptide of genotype 1 comprises an amino acid sequence having at least 90%, or at least 95%, amino acid sequence identity to an H77 E1 polypeptide, and the HCV E2 polypeptide of genotype 1 comprises an amino acid sequence having at least 90%, or at least 95%, amino acid sequence identity to an H77 E2 polypeptide;
b) the HCV E1 polypeptide of genotype 2 comprises an amino acid sequence having at least 90%, or at least 95%, % amino acid sequence identity to a J6 E1 polypeptide, and the HCV E2 polypeptide of genotype 2 comprises an amino acid sequence having at least 90%, or at least 95%, amino acid sequence identity to a J6 E2 polypeptide; and
c) the HCV E1 polypeptide of genotype 3 comprises an amino acid sequence having at least 90%, or at least 95%, amino acid sequence identity to an S52 E1 polypeptide, and the HCV E2 polypeptide of genotype 3 comprises an amino acid sequence having at least 90%, or at least 95%, amino acid sequence identity to an S52 E2 polypeptide.

Aspect 66. The composition of aspect 64 or aspect 65, wherein the polypeptide-encoding region of the RNA molecule comprises a nucleotide sequence encoding an HCV Core polypeptide.

Aspect 67. The composition of aspect 66, wherein:
a) the polypeptide-encoding region of the RNA molecule comprises:
   i) a nucleotide sequence encoding an HCV Core-E1-E2 polyprotein of genotype 1;
   ii) a first IRES; and
   iii) one or more nucleotide sequences encoding the one or more heterologous polypeptides or the fusion polypeptide; or
b) the polypeptide-encoding region of the RNA molecule comprises:
   i) a nucleotide sequence encoding an HCV Core-E1-E2 polyprotein of genotype 2;
   ii) a first IRES; and
   iii) one or more nucleotide sequences encoding the one or more heterologous polypeptides or the fusion polypeptide; or
c) the polypeptide-encoding region of the RNA molecule comprises:
   i) a nucleotide sequence encoding an HCV Core-E1-E2 polyprotein of genotype 3;
   ii) a first IRES; and
   iii) one or more nucleotide sequences encoding the one or more heterologous polypeptides or the fusion polypeptide; or
d) the polypeptide-encoding region of the RNA molecule comprises:
   i) a nucleotide sequence encoding an HCV Core-E1-E2 polyprotein of genotype 4;
   ii) a first IRES; and
   iii) one or more nucleotide sequences encoding the one or more heterologous polypeptides or the fusion polypeptide; or
e) the polypeptide-encoding region of the RNA molecule comprises:
   i) a nucleotide sequence encoding an HCV Core-E1-E2 polyprotein of genotype 5;
   ii) a first IRES; and
   iii) one or more nucleotide sequences encoding the one or more heterologous polypeptides or the fusion polypeptide; or
f) the polypeptide-encoding region of the RNA molecule comprises:
   i) a nucleotide sequence encoding an HCV Core-E1-E2 polyprotein of genotype 6;
   ii) a first IRES; and
   iii) one or more nucleotide sequences encoding the one or more heterologous polypeptides or the fusion polypeptide; or
g) the polypeptide-encoding region of the RNA molecule comprises:
   i) a nucleotide sequence encoding HCV E1 and HCV E2 polypeptides of genotype 1a, optionally wherein the HCV E1 and E2 polypeptides comprise amino acid sequences having at least 80% amino acid sequence identity to the E1 and E2 amino acid sequences depicted in FIG. 32A;
   ii) a first IRES;
   iii) a nucleotide sequence encoding a TP465 polypeptide comprising an amino acid sequence having at least 80% amino acid sequence identity to the TP465 polypeptide amino acid sequence depicted in FIG. 32B, FIG. 33B, or FIG. 34B;
   iv) a second IRES; and
   v) a nucleotide sequence encoding a TP156 polypeptide comprising an amino acid sequence having at least 80% amino acid sequence identity to the TP156 polypeptide amino acid sequence depicted in FIG. 32C; or
h) the polypeptide-encoding region of the RNA molecule comprises:
   i) a nucleotide sequence encoding HCV E1 and HCV E2 polypeptides of genotype 3a, optionally wherein the HCV E1 and E2 polypeptides comprise amino acid sequences having at least 80% amino acid sequence identity to the E1 and E2 amino acid sequences depicted in FIG. 33A;
   ii) a first IRES;
   iii) a nucleotide sequence encoding a TP465 polypeptide comprising an amino acid sequence having at least 80% amino acid sequence identity to the TP465 polypeptide amino acid sequence depicted in FIG. 32B, FIG. 33B, or FIG. 34B;
   iv) a second IRES; and
   v) a nucleotide sequence encoding a TP48 polypeptide comprising an amino acid sequence having at least 80% amino acid sequence identity to the TP48 polypeptide amino acid sequence depicted in FIG. 33C; or
j) the polypeptide-encoding region of the RNA molecule comprises:
   i) a nucleotide sequence encoding HCV E1 and HCV E2 polypeptides of genotype 2a, optionally wherein the HCV E1 and E2 polypeptides comprise amino acid sequences having at least 80% amino acid sequence identity to the E1 and E2 amino acid sequences depicted in FIG. 34A;
   ii) a first IRES;
   iii) a nucleotide sequence encoding a TP465 polypeptide comprising an amino acid sequence having at least 80% amino acid sequence identity to the TP465 polypeptide amino acid sequence depicted in FIG. 32B, FIG. 33B, or FIG. 34B;
   iv) a second IRES; and
   v) a nucleotide sequence encoding a TP65 polypeptide comprising an amino acid sequence having at least 80% amino acid sequence identity to the TP65 polypeptide amino acid sequence depicted in FIG. 34C.

Aspect 68. The composition of aspect 67, wherein:
a) the HCV Core-E1-E2 polyprotein of genotype 1 comprises an amino acid sequence having at least 90%, or at least 95%, amino acid sequence identity to an H77 HCV Core-E1-E2 polyprotein;
b) the HCV Core-E1-E2 polyprotein of genotype 2 comprises an amino acid sequence having at least 90%, or at least 95%, amino acid sequence identity to a J6 HCV Core-E1-E2 polyprotein; and
c) the HCV Core-E1-E2 polyprotein of genotype 3 comprises an amino acid sequence having at least 90%, or at least 95%, amino acid sequence identity to an S52 HCV Core-E1-E2 polyprotein.

Aspect 69. The composition of any one of aspects 17 to 28, wherein the composition comprises a single type of RNA molecule, and wherein:
a) the polypeptide-encoding region of the RNA molecule comprises a nucleotide sequence encoding a fusion polypeptide comprising:
   i) HCV E1 and HCV2 E2 polypeptides of genotype 1; and
   ii) the fusion polypeptide comprising two or more heterologous polypeptides comprising a T-cell epitope present in an HCV protein other than E1 and E2; or
b) the polypeptide-encoding region of the RNA molecule comprises a nucleotide sequence encoding a fusion polypeptide comprising:
   i) HCV E1 and HCV2 E2 polypeptides of genotype 2; and
   ii) the fusion polypeptide comprising two or more heterologous polypeptides comprising a T-cell epitope present in an HCV protein other than E1 and E2; or
c) the polypeptide-encoding region of the RNA molecule comprises a nucleotide sequence encoding a fusion polypeptide comprising:
   i) HCV E1 and HCV2 E2 polypeptides of genotype 3; and
   ii) the fusion polypeptide comprising two or more heterologous polypeptides comprising a T-cell epitope present in an HCV protein other than E1 and E2; or
d) the polypeptide-encoding region of the RNA molecule comprises a nucleotide sequence encoding a fusion polypeptide comprising:
   i) HCV E1 and HCV2 E2 polypeptides of genotype 4; and
   ii) the fusion polypeptide comprising two or more heterologous polypeptides comprising a T-cell epitope present in an HCV protein other than E1 and E2; or
e) the polypeptide-encoding region of the RNA molecule comprises a nucleotide sequence encoding a fusion polypeptide comprising:
   i) HCV E1 and HCV2 E2 polypeptides of genotype 5; and
   ii) the fusion polypeptide comprising two or more heterologous polypeptides comprising a T-cell epitope present in an HCV protein other than E1 and E2; or
f) the polypeptide-encoding region of the RNA molecule comprises a nucleotide sequence encoding a fusion polypeptide comprising:
   i) HCV E1 and HCV2 E2 polypeptides of genotype 6; and
   ii) the fusion polypeptide comprising two or more heterologous polypeptides comprising a T-cell epitope present in an HCV protein other than E1 and E2.

Aspect 70. The composition of aspect 69, wherein:
a) the HCV E1 polypeptide of genotype 1 comprises an amino acid sequence having at least 90%, or at least 95%, amino acid sequence identity to an H77 E1 polypeptide, and the HCV E2 polypeptide of genotype 1 comprises an amino acid sequence having at least 90%, or at least 95%, amino acid sequence identity to an H77 E2 polypeptide;
b) the HCV E1 polypeptide of genotype 2 comprises an amino acid sequence having at least 90%, or at least 95%, amino acid sequence identity to a J6 E1 polypeptide, and the HCV E2 polypeptide of genotype 2 comprises an amino acid sequence having at least 90%, or at least 95%, amino acid sequence identity to a J6 E2 polypeptide; and
c) the HCV E1 polypeptide of genotype 3 comprises an amino acid sequence having at least 90%, or at least 95%, amino acid sequence identity to an S52 E1 polypeptide, and the HCV E2 polypeptide of genotype 3 comprises an amino acid sequence having at least 90%, or at least 95%, amino acid sequence identity to an S52 E2 polypeptide.

Aspect 71. The composition of aspect 69, wherein:
a) the HCV E1 polypeptide encoded by the RNA molecule is a soluble HCV E1 polypeptide; and/or
b) the HCV E2 polypeptide encoded by the RNA molecule is a soluble HCV E2 polypeptide.

Aspect 72. The composition of aspect 71, wherein:
a) the soluble HCV E1 polypeptide of genotype 1 comprises an amino acid sequence having at least 90%, or at least 95%, amino acid sequence identity to a soluble H77 E1 polypeptide, and the soluble HCV E2 polypeptide of genotype 1 comprises an amino acid sequence having at least 90%, or at least 95%, amino acid sequence identity to a soluble H77 E2 polypeptide;
b) the soluble HCV E1 polypeptide of genotype 2 comprises an amino acid sequence having at least 90%, or at least 95%, amino acid sequence identity to a soluble J6 E1 polypeptide, and the soluble HCV E2 polypeptide of genotype 2 comprises an amino acid sequence having at least 90%, or at least 95%, amino acid sequence identity to a soluble J6 E2 polypeptide; and
c) the soluble HCV E1 polypeptide of genotype 3 comprises an amino acid sequence having at least 90%, or at least 95%, amino acid sequence identity to a soluble S52 E1 polypeptide, and the soluble HCV E2 polypeptide of genotype 3 comprises an amino acid sequence having at least 90%, or at least 95%, amino acid sequence identity to a soluble S52 E2 polypeptide.

Aspect 73. The composition of any one of aspects 69-72, wherein the polypeptide-encoding region of the RNA molecule comprises a nucleotide sequence encoding an HCV Core polypeptide.

Aspect 74. The composition of aspect 73, wherein:
a) the polypeptide-encoding region of the RNA molecule comprises a nucleotide sequence encoding a fusion polypeptide comprising:
   i) an HCV Core-E1-E2 polyprotein of genotype 1; and
   ii) the fusion polypeptide comprising two or more heterologous polypeptides comprising a T-cell epitope present in an HCV protein other than E1 and E2; or
b) the polypeptide-encoding region of the RNA molecule comprises a nucleotide sequence encoding a fusion polypeptide comprising:
   i) an HCV Core-E1-E2 polyprotein of genotype 2; and
   ii) the fusion polypeptide comprising two or more heterologous polypeptides comprising a T-cell epitope present in an HCV protein other than E1 and E2; or
c) the polypeptide-encoding region of the RNA molecule comprises a nucleotide sequence encoding a fusion polypeptide comprising:
   i) an HCV Core-E1-E2 polyprotein of genotype 3; and
   ii) the fusion polypeptide comprising two or more heterologous polypeptides comprising a T-cell epitope present in an HCV protein other than E1 and E2; or
d) the polypeptide-encoding region of the RNA molecule comprises a nucleotide sequence encoding a fusion polypeptide comprising:
   i) an HCV Core-E1-E2 polyprotein of genotype 4; and
   ii) the fusion polypeptide comprising two or more heterologous polypeptides comprising a T-cell epitope present in an HCV protein other than E1 and E2; or
e) the polypeptide-encoding region of the RNA molecule comprises a nucleotide sequence encoding a fusion polypeptide comprising:
   i) an HCV Core-E1-E2 polyprotein of genotype 5; and
   ii) the fusion polypeptide comprising two or more heterologous polypeptides comprising a T-cell epitope present in an HCV protein other than E1 and E2; or
f) the polypeptide-encoding region of the RNA molecule comprises a nucleotide sequence encoding a fusion polypeptide comprising:
   i) an HCV Core-E1-E2 polyprotein of genotype 6; and
   ii) the fusion polypeptide comprising two or more heterologous polypeptides comprising a T-cell epitope present in an HCV protein other than E1 and E2.

Aspect 75. The composition of aspect 74, wherein:
a) the HCV Core-E1-E2 polyprotein of genotype 1 comprises an amino acid sequence having at least 90%, or at least 95%, amino acid sequence identity to an H77 Core-E1-E2 polyprotein;
b) the HCV Core-E1-E2 polyprotein of genotype 2 comprises an amino acid sequence having at least 90%, or at least 95%, amino acid sequence identity to a J6 Core-E1-E2 polyprotein; and
c) the HCV Core-E1-E2 polyprotein of genotype 3 comprises an amino acid sequence having at least 90%, or at least 95%, amino acid sequence identity to an S52 Core-E1-E2 polyprotein.

Aspect 76. The composition of any one of aspects 64-75, wherein the fusion polypeptide comprising two or more heterologous polypeptides comprising a T-cell epitope present in an HCV protein other than E1 and E2 comprises:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; Xⱼ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; Xₙ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 77. The composition of any one of aspects 64-75, wherein the fusion polypeptide comprising two or more heterologous polypeptides comprising a T-cell epitope present in an HCV protein other than E1 and E2 comprises, in order from N-terminus to C-terminus:
a) a TP50C T-cell epitope polypeptide comprising an amino acid sequence having at least 80% amino acid sequence identity to:
GVYX₁LPRRGPRLGVRX₂TRKX₃SERSQPRGRRQX₄IPKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; Xⱼ is R or K; X₁₁ is S, T, H, or A; X₁₂ is A or G; and X₁₃ is Q or K, wherein the TP50C T-cell epitope polypeptide has a length of from 40 amino acids to 50 amino acids;
b) a TP35-NS3 T-cell epitope polypeptide comprising an amino acid sequence having at least 80% amino acid sequence identity to:
KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
c) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids; and
d) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₈ is V or T; X₉ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acid.

Aspect 78. The composition of any one of aspects 17-28, wherein the one or more RNA molecules comprises a first RNA molecule, a second RNA molecule, and a third RNA molecule, wherein:
a) the first RNA molecule comprises:
   i) a nucleotide sequence encoding HCV E1 and HCV E2 polypeptides of a first genotype;
   ii) a first IRES;
   iii) a nucleotide sequence encoding a TP465 polypeptide comprising an amino acid sequence having at least 80% amino acid sequence identity to the TP465 polypeptide amino acid sequence depicted in FIG. 32B, FIG. 33B, or FIG. 34B;
   iv) a second IRES; and
   v) a nucleotide sequence encoding a TP156 polypeptide comprising an amino acid sequence having at least 80% amino acid sequence identity to the TP156 polypeptide amino acid sequence depicted in FIG. 32C;
b) the second RNA molecule comprises:
   i) a nucleotide sequence encoding HCV E1 and/or an HCV E2 polypeptide of a second genotype;
   ii) a first IRES;
   iii) a nucleotide sequence encoding a TP465 polypeptide comprising an amino acid sequence having at least 80% amino acid sequence identity to the TP465 polypeptide amino acid sequence depicted in FIG. 32B, FIG. 33B, or FIG. 34B;
   iv) a second IRES; and
   v) a nucleotide sequence encoding a TP48 polypeptide comprising an amino acid sequence having at least 80% amino acid sequence identity to the TP48 polypeptide amino acid sequence depicted in FIG. 33C; and
c) the third RNA molecule comprises:
   i) a nucleotide sequence encoding HCV E1 and/or an HCV E2 polypeptide of a second genotype;
   ii) a first IRES;
   iii) a nucleotide sequence encoding a TP465 polypeptide comprising an amino acid sequence having at least 80% amino acid sequence identity to the TP465 polypeptide amino acid sequence depicted in FIG. 32B, FIG. 33B, or FIG. 34B;
   iv) a second IRES; and
   v) a nucleotide sequence encoding a TP65 polypeptide comprising an amino acid sequence having at least 80% amino acid sequence identity to the TP65 polypeptide amino acid sequence depicted in FIG. 34C.

Aspect 79. The composition of aspect 78, wherein:
a) the nucleotide sequence encoding HCV E1 and HCV E2 polypeptides of the first genotype encodes HCV E1 and E2 polypeptides having at least 80% amino acid sequence identity to the E1 and E2 amino acid sequences depicted in FIG. 32A;
b) the nucleotide sequence encoding HCV E1 and HCV E2 polypeptides of the second genotype encodes HCV E1 and E2 polypeptides having at least 80% amino acid sequence identity to the E1 and E2 amino acid sequences depicted in FIG. 33A; and
c) the nucleotide sequence encoding HCV E1 and HCV E2 polypeptides of the third genotype encodes HCV E1 and E2 polypeptides having at least 80% amino acid sequence identity to the E1 and E2 amino acid sequences depicted in FIG. 34A.

Aspect 80. The composition of any one of aspects 1-79, wherein the RNA is a self-amplifying RNA comprising a nucleotide sequence encoding an RNA-dependent RNA polymerase (RdRP).

Aspect 81. The composition of aspect 80, wherein the self-amplifying RNA is a cis-replicon.

Aspect 82. The composition of aspect 80 or aspect 81, wherein the self-amplifying RNA is a recombinant Venezuelan equine encephalitis virus, a Sindbis virus, or a recombinant Semliki Forest virus.

Aspect 83. The composition of any one of aspects 1-82, wherein the composition is in the form of a lipid nanoparticle.

Aspect 84. The composition of any one of aspects 1-82, wherein the composition is in the form of a liposome.

Aspect 85. The composition of any one of aspects 1-82, wherein the composition comprises one or more lipids selected from cationic lipids, neutral lipids, anionic lipids, helper lipids, and stealth lipids.

Aspect 86. The composition of any one of aspects 1-2, wherein the composition comprises an HCV Core polypeptide and wherein the one or more RNA molecules are within a virus-like particle comprising the HCV Core polypeptide.

Aspect 87. A method of inducing an immune response to hepatitis C virus (HCV) in an individual, the method comprising administering to the individual an effective amount of a composition of any one of aspects 1-86.

Aspect 88. The method of aspect 87, comprising:
a) administering a first dose of the composition at a first time; and
b) administering a second dose of the composition at a second time.

Aspect 89. The method of aspect 88, wherein the second time is from a period of time of from about 1 week to about 6 months from the first time.

Aspect 90. The method of any one of aspects 87-89, wherein said administering is intramuscular administration.

Aspect 91. The method of any one of aspects 87-89, wherein said administering is subcutaneous administration.

Aspect 92. The method of any one of aspects 87-91, comprising administering one or more heterologous polypeptides comprising a T-cell epitope present in an HCV protein other than E1 and E2.

Aspect 93. The method of any one of aspect 87-92, comprising administering an HCV E1 polypeptide and/or an HCV E2 polypeptide.

Aspect 94. The method of aspect 88, wherein:
a) the first dose comprises one or more RNA molecules comprising a polypeptide-encoding region comprising one or more nucleotide sequences encoding a hepatitis C virus (HCV) E1 polypeptide and/or an HCV E2 polypeptide of a first genotype; and
b) the second dose comprises one or more RNA molecules comprising a polypeptide-encoding region comprising one or more nucleotide sequences encoding a hepatitis C virus (HCV) E1 polypeptide and/or an HCV E2 polypeptide of a second genotype.

Aspect 95. The method of aspect 94, wherein:
a) the first dose comprises a composition of any one of aspects 64-76; and
b) the second dose comprises a composition of any one of aspects 64-76.

Aspect 96. The method of aspect 94, wherein:
a1) the first dose comprises a composition of any one of aspects 64-76, wherein the HCV E1 and/or HCV E2 polypeptide are of genotype 1; and
b1) the second dose comprises a composition of any one of aspects 64-76, wherein the HCV E1 and/or HCV E2 polypeptide are of genotype 3; or
a2) the first dose comprises a composition of any one of aspects 64-76, wherein the HCV E1 and/or HCV E2 polypeptide are of genotype 1; and
b2) the second dose comprises a composition of any one of aspects 64-76, wherein the HCV E1 and/or HCV E2 polypeptide are of genotype 4; or
a3) the first dose comprises a composition of any one of aspects 64-76, wherein the HCV E1 and/or HCV E2 polypeptide are of genotype 1; and
b3) the second dose comprises a composition of any one of aspects 64-76, wherein the HCV E1 and/or HCV E2 polypeptide are of genotype 5; or
a4) the first dose comprises a composition of any one of aspects 64-76, wherein the HCV E1 and/or HCV E2 polypeptide are of genotype 1; and
b4) the second dose comprises a composition of any one of aspects 64-76, wherein the HCV E1 and/or HCV E2 polypeptide are of genotype 6; or
a5) the first dose comprises a composition of any one of aspects 64-76, wherein the HCV E1 and/or HCV E2 polypeptide are of genotype 1; and
b5) the second dose comprises a composition of any one of aspects 64-76, wherein the HCV E1 and/or HCV E2 polypeptide are of genotype 2.

Aspect 97. The method of any one of aspect 88-94, comprising administering at least a third dose of the composition at a third time.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A composition comprising:
a) one or more RNA molecules, wherein the one or more RNA molecules comprises:
i) at least one 5′ cap structure and/or a 5′ untranslated region (5′ UTR) and/or a 5′ internal ribosome entry site (IRES);
ii) a polypeptide-encoding region comprising one or more nucleotide sequences encoding a hepatitis C virus (HCV) E1 polypeptide and/or an HCV E2 polypeptide; and
b) one or more of: i) a lipid; ii) a polymer; iii) a peptide; iv) a viral-like particle; and v) a cationic nanoemulsion.

2. The composition of claim 1, wherein the one or more RNA molecules comprises a 5′ cap structure and a 5′ UTR, wherein the 5′UTR is 3′ of the 5′ cap structure.

3. The composition of claim 1, wherein the one or more RNA molecules comprises a 3′ UTR and/or a 3′ tailing sequence.

4. The composition of claim 3, wherein the 3′ tailing sequence is a poly(adenosine) (poly-A) sequence.

5. The composition of claim 1, wherein the one or more RNA molecules include at least one 5′ cap structure, and wherein the at least one 5′ cap structure is selected from the group consisting of Cap0, Cap1, ARCA, inosine, N1-methyl-guanosine, 2′fluoro-guanosine, 7-deaza-guanosine, 8-oxo-guanosine, 2-amino-guanosine, LNA-guanosine, and 2-azido-guanosine.

6. The composition of claim 1, wherein the one or more RNA molecules comprises one or more of: a nucleoside base modification, a sugar modification, and a backbone modification.

7. The composition of claim 1, wherein at least one of the one or more RNA molecules comprises a nucleotide of Formula I: 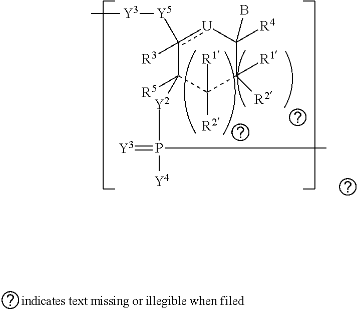
wherein:
U is O, S, N(R^{U})ₙᵤ, or C(R^{U})ₙᵤ, where nu is an integer nu is an integer from 0 to 2 and each R^{U} is, independently, H, halo, or optionally substituted alkyl; [symbol]
is a single or double bond; [symbol]
is a single bond or absent;
each of R^{1′}, R^{2′}, R^{1″}, R^{2″}, R³, R⁴, and R⁵ is, independently, H, halo, hydroxy, thiol, optionally substituted alkyl, optionally substituted alkoxy, optionally substituted alkenyloxy, optionally substituted alkynyloxy, optionally substituted aminoalkoxy, optionally substituted alkoxyalkoxy, optionally substituted hydroxyalkoxy, optionally substituted amino, azido, optionally substituted aryl, optionally substituted aminoalkyl, or absent; wherein the combination of R³ with one or more of R^{1′}, R^{1″}, R^{2′}, R^{2″}, or R⁵ can join together to form optionally substituted alkylene or optionally substituted heteroalkylene and, taken together with the carbons to which they are attached, provide an optionally substituted heterocyclyl; wherein the combination of R⁵ with one or more of R^{1′}, R^{1″}, R^{2′}, or R^{2″} can join together to form optionally substituted alkylene or optionally substituted heteroalkylene and, taken together with the carbons to which they are attached, provide an optionally substituted heterocyclyl; and wherein the combination of R⁴ and one or more of R^{1′}, R^{1″}, R^{2′}, R^{2″}, R³, or R⁵ can join together to form optionally substituted alkylene or optionally substituted heteroalkylene and, taken together with the carbons to which they are attached, provide an optionally substituted heterocyclyl;
each of Y¹, Y², and Y³, is, independently, O, S, —NR^{N1}—, optionally substituted alkylene, or optionally substituted heteroalkylene, wherein R^{N}1 is H, optionally substituted alkyl, optionally substituted alkenyl, optionally substituted alkynyl, optionally substituted aryl, or absent;
each Y⁴ is, independently, H, hydroxy, thiol, optionally substituted alkyl, optionally substituted alkenyl, optionally substituted alkynyl, optionally substituted alkoxy, optionally substituted alkenyloxy, optionally substituted alkynyloxy, optionally substituted thioalkoxy, optionally substituted alkoxyalkoxy, or optionally substituted amino;
each Y⁵ is, independently, O, S, optionally substituted alkylene, or optionally substituted heteroalkylene;
n is an integer from 1 to 100,000; and
B is a nucleobase, wherein the combination of B and R^{1′}, the combination of B and R^{2′}, the combination of B and R¹, or the combination of B and R^{2′} can, taken together with the carbons to which they are attached, optionally form a bicyclic group or wherein the combination of B, R^{1″}, and R³ or the combination of B, R^{2″}, and R³ can optionally form a tricyclic or tetracyclic group.

8. The composition of claim 1, wherein the composition comprises a lipid, and wherein the lipid is selected from DLin-DMA, DLin-K-DMA, DLin-KC2-DMA, 98N12-5, C12-200, DLin-MC3-DMA, DODMA, DSDMA, DLenDMA, reLNPs, PLGA, a PEGylated lipid, and mixtures of two or more of the foregoing.

9. The composition of claim 1, wherein the composition comprises a polymer, wherein the polymer is selected from polyethylene glycol, polyglycolide, polyvinyl alcohol, polyvinyl pyrrolidone, polylactide, poly(lactide-co-glycolide) (PLGA), polycaprolactone, polysorbate, polyethylene oxide, polypropylene oxide, poly(ethylene oxide-co-propylene oxide), poloxamer, poloxamine, poly(oxyethylated) glycerol, poly(oxyethylated) sorbitol, poly(oxyethylated) glucose, polyethyleneimine, polyamidoamine (PAMAM) dendrimer, and block copolymer poly(ethylene glycol)-block-poly(lactic-co-glycolic acid) (PEG-b-PLGA).

10. (canceled)

11. The composition of claim 1, wherein the HCV E1 polypeptide is a soluble HCV E1 polypeptide and/or the HCV E2 polypeptide is a soluble HCV E2 polypeptide.

12. (canceled)

13. (canceled)

14. (canceled)

15. The composition of claim 1, wherein the polypeptide-encoding region comprises a nucleotide sequence encoding an HCV Core polypeptide.

16. (canceled)

17. The composition of claim 1, wherein the polypeptide-encoding region comprises:
a) one or more nucleotide sequences encoding one or more heterologous polypeptides comprising a T-cell epitope present in an HCV protein other than E1 and E2; or
b) a nucleotide sequence encoding a fusion polypeptide comprising two or more heterologous polypeptides comprising a T-cell epitope present in an HCV protein other than E1 and E2.

18. The composition of claim 17, wherein the RNA comprises:
a) an internal ribosome entry site (IRES) between i) the one or more nucleotide sequences encoding the HCV E1 and/or E2 polypeptides; and ii) the one or more nucleotide sequences encoding the one or more heterologous polypeptides or the nucleotide sequence encoding the fusion polypeptide; or
b) a nucleotide sequence encoding a self-cleaving peptide between i) the one or more nucleotide sequences encoding the HCV E1 and/or E2 polypeptides; and ii) the one or more nucleotide sequences encoding the one or more heterologous polypeptides or the nucleotide sequence encoding the fusion polypeptide.

19. The composition of claim 17, wherein the one or more heterologous polypeptides is selected from:
i) a TP35-NS3 T-cell epitope polypeptide comprising an amino acid sequence having at least 80% amino acid sequence identity to: KSTKVPX₁AYX₂X₃QGYX₄VLVLNPSVAATLGFGX₅X₆X₇SX₈ (SEQ ID NO:219), wherein X₁ is A or V; X₂ is A or V; X₃ is A or S; X₄ is K or N; X₅ is A or S; X₆ is Y or F; X₇ is M or L; and X₈ is K or R, wherein the TP35-NS3 T-cell epitope polypeptide has a length of from 28 amino acids to 35 amino acids;
ii) a TP50C T-cell epitope polypeptide comprising an amino acid sequence having at least 80% amino acid sequence identity to: GVYX₁LPRRGPRLGVRX₂TRKX₃SERSQPRGRRQX₄PKX₅X₆X₇X₈X₉GX₁₀X₁₁WX₁₂X₁₃PGYP (SEQ ID NO:234), where X₁ is L or V; X₂ is A or G; X₃ is T or S; X₄ is P or R; X₅ is A or D; X₆ is R or A; X₇ is R, Q, or S; X₈ is S or P; X₉ is E, T, or Q; X₁₀ is R or K; X₁ is S, T, H, or A; X₁₂ is A or G; and X₁₃ is Q or K, wherein the TP50C T-cell epitope polypeptide has a length of from 40 amino acids to 50 amino acids;
iii) a TP23 T-cell epitope polypeptide comprising an amino acid sequence having at least 80% amino acid sequence identity to: DVVVX₁X₂TDALMTGX₃TGDFDSVID (SEQ ID NO:257), wherein X₁ is V or C; X₂ is A or S; and X₃ is F or Y, wherein the TP23 T-cell epitope polypeptide has a length of from 18 amino acids to 23 amino acids;
iv) a TP27 T-cell epitope polypeptide comprising an amino acid sequence having at least 80% amino acid sequence identity to: X₁X₂X₃X₄KGGRHLIFCHSKKKCDEX₅AX₆X₇LX₈ (SEQ ID NO:273), where X₁ is L or I; X₂ is E, A, S, V, or Q; X₃ is Q, T, Y, F, or L; X₄ is I or L; X₅ is L or I; X₆ is A, K, or S; X₇ is K, Q, or A; and X₈ is T, R, or S, wherein the TP27 T-cell epitope polypeptide has a length of from 22 amino acids to 27 amino acids;
v) a TP35-NS4 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀ (SEQ ID NO:288), wherein X₁ is I or V; X₂ is L or I; X₃ is R or K; X₄ is V, I, or T; X₅ is P, Q, or T; X₆ is G, A, or S; X₇ is A or V; X₅ is V or T; X₈ is S or A; and X₁₀ is V or I, wherein the TP35-NS4 T-cell epitope polypeptide has a length of from 28 amino acids to 35 amino acids;
vi) a TP42 T-cell epitope polypeptide comprising an amino acid sequence having at least 80% amino acid sequence identity to: X₁X₂X₃GEIPFYGX₄AIPX₅X₆X₇X₈KGGRHLIFCHSKKKCDEX₉AX₁₀X₁₁LX₁₂X₁₃(K)n (SEQ ID NO:314), where X₁ is G, P, or S; X₂ is T, N, Q, H, or S; X₃ is E, T, or D; X₄ is K or R; X₅ is L or I; X₆ is E, A, S, or Q; X₇ is Q, T, Y, F, or L; X₈ is I or L; X₉ is L or I; X₁₀ is A, K, or S; X₁₁ is K, Q, or A; X₁₂ is T, R, or S; and X₁₃ is G or S, wherein n is an integer from 2 to 10, and wherein the TP42 T-cell epitope polypeptide has a length of from 34 amino acids to 52 amino acids;
vii) a TP45 T-cell epitope polypeptide comprising an amino acid sequence having at least 80% amino acid sequence identity to: X₁X₂AVAX₃YRGX₄DVX₅X₆IPX₇X₈GDVVVX₉X₁₀TDALMTGX₁₁TGDFDSVIDX₁₂X₁₃X₁₄(K)n (SEQ ID NO:332), wherein X₁ is L or V; X₂ is N or T; X₃ is Y or F; X₄ is L or V; X₅ is S or A; X₆ is V or I; X₇ is T or A; X₅ is S, Q, or T; X₈ is V or C; X₁₀ is A or S; X₁, is F or Y; X₁₂ is C or K; X₁₃ is N or K; and X₁₄ is V or K, wherein n is an integer from 2 to 10, and wherein the TP45 T-cell epitope polypeptide has a length of from 36 amino acids to 55 amino acids; and
viii) a TP48 T-cell epitope polypeptide comprising an amino acid sequence having at least 80% amino acid sequence identity to: X₁X₂X₃RHX₄GX₅X₆EGX₇X₈QWMNRLIAFASRGNHVX₉PTHYX₁₀X₁₁X₁₂X₁₃DAX₁₄X₁₅X₁₆VX₁₇X₁₈X₁₉L(K)n (SEQ ID NO:339), wherein X₁ is I or V; X₂ is L or I; X₃ is R or K; X₄ is V, I, or T; X₅ is P, Q, or T; X₆ is G, A, or S; X₇ is A or V; X₅ is V or T; X₈ is S or A; X₁₀ is V or I; X₁₁ is P, T, A, or Q, X₁₂ is E or D; X₁₃ is S, T, or D; X₁₄ is S or A; X₁₅ is A, Q, R, or K; X₁₆ is R, K, or X; X₁₇ is T or M; X₁₈ is Q, A, T, or G; and X₁₉ is I, L, or V, wherein n is an integer from 2 to 10, and wherein the TP48 T-cell epitope polypeptide has a length of from 38 amino acids to 58 amino acids;
ix) a TP33 T-cell epitope polypeptide comprising an amino acid sequence having at least 80% amino acid sequence identity to: HSKKKCDELAX₁X₂LX₃X₄X₅GX₆NAVAYYRGLDVSX₇IP (SEQ ID NO:366), where X₁ is A or S; X₂ is K or A; X₃ is V, S, R, or T; X₄ is A or G; X₅ is L or M; X₆ is I, L, or V; and X₇ is V or I; wherein the TP33 T-cell epitope polypeptide has a length of from 30 amino acids to 36 amino acids;
x) a TP42-2 T-cell epitope polypeptide comprising an amino acid sequence having at least 80% amino acid sequence identity to: KGGRHLIFCHSKKKCDELAX₁X₂LX₃X₄X₅GX₆NAVAYYRGLDVSX₇IP (SEQ ID NO:371), where X₁ is A or S; X₂ is K or A; X₃ is V, S, R, or T; X₄ is A or G; X₅ is L or M; X₆ is I, L, or V; and X₇ is V or I; wherein the TP42-2 T-cell epitope polypeptide has a length of from 38 amino acids to 46 amino acids;
xi) a TP240 T-cell epitope polypeptide comprising an amino acid sequence having at least 80% amino acid sequence identity to: SYQVGYLHAPTGSGKSTKVPAAYAAQGYKVLVLNPSVAATLGFGAYMSKAHGIDPNIRTGVRTVTTGAPITYSTYGKF LADGGCSGGAYDIIICDECHSX₁DATTILGIGTVLDQAETAGARLVVLATATPPGSVTVPHPNIEEVALGX₂EGEIPFYGK AIPLX₃X₄IKGGRHLIFCHSKKKCDELAAKLRGMGLNAVAYYRGLDVSVIPTX₅GDVVVVATDALMTGYTGDFDSVIDC NVAVTQT (SEQ ID NO:379), where X₁ is T, V, or Q, X₂ is T, N, Q, H, or S, X₃ is E, S, or A, X₄ is V, T, Y, F, or L, and X₅ is S, Q, or T; wherein the TP240 T-cell epitope polypeptide has a length of from 240 amino acids to 245 amino acids;
xii) a TP65 T-cell epitope polypeptide comprising an amino acid sequence having at least 80% amino acid sequence identity to: TPIDTTIMAKNEVFCVDPX₆KGGRKPARLIVYPDLGVRVCEKMALYDVVQKLPQAVMGX₇SYGFQYS (SEQ ID NO:375), where X₆ is E, T, or V, and X₇ is S, A, or P; and wherein the TP65 T-cell epitope polypeptide has a length of from 65 amino acids to 70 amino acids;
xiii) a TP156 T-cell epitope polypeptide comprising an amino acid sequence having at least 80% amino acid sequence identity to the TP156 amino acid sequence depicted in FIG. 32C, where the TP156 T-cell epitope polypeptide has a length of from 150 amino acids to 160 amino acids; and
xiv) a TP465 T-cell epitope polypeptide comprising an amino acid sequence having at least 80% amino acid sequence identity to the TP465 amino acid sequence depicted in any one of FIGS. 32B, 33B, and 34B, where the TP465 T-cell epitope polypeptide has a length of from 460 amino acids to 470 amino acids, or
wherein fusion polypeptide comprises from 2 to 10 polypeptides selected from the group consisting of the polypeptides set forth in: i)-xiv).

20. The composition of claim 17, wherein the polypeptide-encoding region comprises one or more nucleotide sequences encoding two or more heterologous polypeptides comprising a T-cell epitope present in an HCV protein other than E1 and E2.

21. The composition of claim 20, wherein the mRNA comprises:
a) a first internal ribosome entry site (IRES) between i) the one or more nucleotide sequences encoding the HCV E1 and/or E2 polypeptides; and ii) the nucleotide sequence encoding the first of the two or more heterologous polypeptides; and
b) a second IRES between i) the nucleotide sequence encoding the first of the two or more heterologous polypeptides; and ii) the nucleotide sequence encoding the second of the two or more heterologous polypeptides,
wherein the first IRES and the second IRES are the same or different.

22. (canceled)

23. (canceled)

24. The composition of claim 1, wherein the HCV E1 and/or E2 polypeptide is from HCV genotype 1, optionally genotype 1a; wherein the HCV E1 and/or E2 polypeptide is from HCV genotype 2, optionally genotype 2a; or wherein the HCV E1 and/or E2 polypeptide is from HCV genotype 3, optionally genotype 3a.

25.-79. (canceled)

80. The composition of claim 1, wherein the RNA is a self-amplifying RNA comprising a nucleotide sequence encoding an RNA-dependent RNA polymerase (RdRP).

81. (canceled)

82. (canceled)

83. The composition of claim 1, wherein the composition is in the form of a lipid nanoparticle or a liposome.

84. (canceled)

85. (canceled)

86. (canceled)

87. A method of inducing an immune response to hepatitis C virus (HCV) in an individual, the method comprising administering to the individual an effective amount of a composition of claim 1.

88.-97. (canceled)


SEQUENCE LISTING (397 sequences)

SEQ ID NO: 1 - protein, 746 aa, Hepacivirus C
  VARIANT 399: note = X can be any naturally occurring amino acid
  VARIANT 408: note = X can be any naturally occurring amino acid
  VARIANT 524: note = X can be any naturally occurring amino acid
  VARIANT 580: note = X can be any naturally occurring amino acid
  VARIANT 641: note = X can be any naturally occurring amino acid
  VARIANT 709: note = X can be any naturally occurring amino acid
  VARIANT 733: note = X can be any naturally occurring amino acid
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLMSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLRMAWDMMMNWSPTTALVVAQLLRIPQAILDMIAGAHWGVLAGIAYFSMVGNWAKVLVVLLLFAGVDAETHVTGGSAARATSGXASLFSPGAXQNIQLINTNGSWHINRTALNCNDSLDTGWVAGLFYYHKFNSSGCPERMASCRPLADFDQGWGPISYANGSGPDQRPYCWHYPPKPCGIVPAQQVCGPVYCFTPSPVVVGTTDRLGXPTYNWGENETDVLVLNNTRPPLGNWFGCTWMNSTGFTKVCGAPPCNIGGVGNNTLXCPTDCFRKHPEATYSRCGSGPWLTPRCLVDYPYRLWHYPCTVNYTIFKVRMYVGGVEHRLXAACNWTRGERCDLDDRDRSELSPLLLSTTQWQVLPCSFTTLPALSTGLIHLHQNIVDVQYLYGVGSSXVSWAIKWEYVILLFLLLADARICXCLWMMLLISQAEA

SEQ ID NO: 2 - protein, 746 aa, Hepacivirus C
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSTGLYHVTNDCPNSSIVYEAADAILHTPGCVPCVREGNTSRCWVAMTPTVATRDGKLPTTQLRRHIDLLVGSATLCSALYVGDLCGSIFLVGQMFTFSPRRHWTTQDCNCSLYPGHITGHRMAWDMMMNWSPTAALITAQLLRIPQAILDMIAGAHWGVLAGIAYFSMVGNWAKVLVVLLLFAGVDAQTHVTGGRAAHITAGLTSLFSPGPSQKLQLVNTNGSWHINSTALNCNDSLKTGWIAGLLYSYKFNSSGCPERLASCRRLTDFAQGWGPISHANGSGPDERPYCWHYPPRPCGIVPAKSVCGPVYCFTPSPVVVGTTDKSGAPTYNWGENDWDVFVLNNTRPPLGNWFGCTWMNSTGFTKVCGAPPCVIGGAGNNTLRCPTDCFRKHPDATYSRCGSGPWITPRCLVDYPYRLWHYPCTVNYSIFKIRMYLGGVEHRLEAACNWTRGERCDLEDRDRSELSPLLLSTTQWQVLPCSFTTLPALSTGLIHLHQNIVDVQYLYGVGSSVASWAIKWDYVVLLFLLLADARICSCLWMMLLISQAEA

SEQ ID NO: 3 - protein, 746 aa, Hepacivirus C
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGSAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 4 - protein, 746 aa, Hepacivirus C
MSTNPKPQRKTKRNTNRRPQDVKFPGGGQIVGGVYLLPRRGPRLGVRAVRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGIYHVTNDCPNSSIVYETADTILHSPGCVPCVREGNASKCWVALAPTVATRDGKLPTTQLRRHIDLLVGSATLCSALYVGDLCGSVFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFAGVDAETYTTGGSVAQAAFGLTSLFRPGPKQDIQLINTNGSWHINRTALNCNASLDTGWVAGLFYYHKFNSSGCPERMASCRSLADFDQGWGPISYANGSGPEHRPYCWHYPPKPCGIVPAQNVCGPVYCFTPSPVVVGTTNKLGVPTYSWGSNETDVLVLNNTRPPLGNWFGCTWMNSSGFTKVCGAPPCVIGGAGNRTLHCPTDCFRKHPEATYSRCGSGPWITPRCLVHYPYRLWHYPCTVNYTMFKVRMYVGGVEHRLEVACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 5 - protein, 746 aa, Hepacivirus C
MSTNPKPQRKTKRNTNRRPQDVKFPGGGQIVGGVYLLPRRGPRLGVRATRKTSERSQPRGRRQPIPKARRPEGRSWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAFQVRNSSGLYHVTNDCPNSSIVYETADTILHSPGCVPCVREDNTSRCWVAVAPTVATRDGRLPTTQLRRHIDLLVGSATLCSALYVGDLCGSVFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFASVDAETYTSGGSVARATAGFAGIFNPGAKQDIQLINTNGSWHINRTALNCNASLDTGWVAGLFYYHKFNSSGCPERMASCKPLAHFAQGWGPISYANGSGPDHRPYCWHYPPKPCGIVPAQNVCGPVYCFTPSPVVVGTTNKLGAPTYNWGSNDTDVFILNNTRPPGGNWFGCTWMNSSGFTKVCGAPPCTIGGVGNNTLLCPTDCFRKHPEATYSRCGSGPWVTPRFLVHYPYRLWHYPCTVNYTLFKVRMYVGGVEHRLEVACNWTRGERCDLDDRDRSELSPLLLSTTQWQVLPCSFTTLPALTTGLIHLHQNIVDVQYLYGVGSSLVSWAIKWEYVILLFLLLADARICSCLWMMLLISQAEA

SEQ ID NO: 6 - protein, 746 aa, Hepacivirus C
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNTSKCWVAVAPTVATRDGKLPTTQLRRHIDLLVGSATLCSALYVGDLCGSVFLVGQLFTFSPRRHWTTQDCNCSIYPGHITGHRMAWDMMMNWSPTAALVTAQLLRVPQAILDMIAGAHWGVLAGIAYFSMAGNWAKVLLVLLLFAGVDAETYTTGGSVARTTRGLASLLQVGPKQDIRLIHTNGSWHINRTALNCNASLDTGWLAGLLYYHKFNSSGCPERMASCRPLADFDQGWGPISYANGSGPEHRPYCWHYPPKPCGIVPAQTVCGPVYCFTPSPVVVGTTNKLGVPTYTWGSNDTDVFVLNNTRPPLGNWFGCTWMNSTGFTKVCGAPPCVIGGVGNNTLHCPTDCFRKHPEATYSRCGSGPWITPRCLVDYPYRLWHYPCTVNYTLFKVRMYVGGVEHRLQAACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 7 - protein, 746 aa, Hepacivirus C
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREDNASRCWVPVAPTVATRDGKLPTTQLRRHIDLLVGSATLCSALYVGDLCGSVFLVSQLFTFSPRRHWTTQDCNCSIYPGHITGHRMAWDMMMNWSPTVALVMAQLLRVPQAILDMIAGAHWGVLAGIAYFSMVGNWAKVLVVLLLFAGVDAQTYVTGGSAARGASGLANLFTPGAKQDIQLINTNGSWHINRTALNCNASLDTGWVAGLFYYHKFNSSGCPERMASCKPLADFDQGWGPIRHANGSGPEHRPYCWHYPPKPCGIVSAQTVCGPVYCFTPSPVVVGTTNRLGVPTYSWGTNDTDVFVLNNTRPPLGNWFGCTWMNSSGFTKVCGAPPCVIGGVGNNTLHCPTDCFRKHPEATYSRCGSGPWITPRCLVHYPYRLWHYPCTVNYTLFKVRMYVGGVEHRLEVACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 8 - protein, 746 aa, Hepacivirus C
MSTNPKPQRKTKRNTNRRPQDVKFPGGGQIVGGVYLLPRRGPRLGVRTTRKTSERSEPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETKDTILHSPGCVPCVREGNVSKCWVPVALTVATRDGNLPTTQLRRHIDLLVGSATLCSALYVGDLCGSVFLVGQLFTFSPRRHWTTQDCNCSMYPGHITGHRMAWDMMMNWSPTSALVVAQLLRVPQAILDMIAGAHWGVLAGLAYFSMVGNWAKVLVVLLLFASVDAGTHVTGGSAAHDVSALAGFFRRGAKQNIQLINTNGSWHVNRTALNCNASLDTGWVAGLLYYHRFNSSGCPERMASCRPLADFDQGWGPITNVDGGGSEYRPYCWHYPPKPCGIEPAQNVCGPVYCFTPSPVVVGTTDKVGVPTYNWGENDTDVFVLNNTRPPLGNWFGCTWMNSSGFVKVCGAPPCIIGGAGNKTLHCPTDCFRKHPDATYSRCGSGPWLTPRCLVDYPYRLWHYPCTVNYTLFKIRMYVGGVEHRLVAACNWTYGERCNLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 9 - protein, 746 aa, Hepacivirus C
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASKCWVAVAPTVATRDGTLPTTQLRRHIDLLVGGATLCSALYVGDLCGSVFLVGQLFTFSPRRHWTTQDCNCSIYPGHITGHRMAWDMMMNWSPTTALIVAQLLRVPQAILDMIAGAHWGVLAGIAYFSMVGNWAKVLVVLLLFAGVDAETHVTGGSAAQVTSRVAGFFNPGPKQNVQLINTNGSWHINRTALNCNASLDTGWVAGLFYHYNFNSSGCPERMASCRPLADFDQGWGPISYANGSGPEHRPYCWHYPPKPCGIVPAQNVCGPVYCFTPSPVVVGTTNRLGVPTYNWGSNDTDVFVLNNTRPPLGNWFGCTWMNSSGFTKVCGAPPCVIGGVGNNTLHCPTDCFRKHPEATYSRCGSGPWITPRCLVHYPYRLWHYPCTVNYTLFKVRMYVGGVEHRLEAACNWTRGERCDLDDRDRSELSPLLLSTTQWQVLPCSFTTLPALTTGLIHLHQNIVDVQYLYGVGSSIVSWAIKWEYVILLFLLLADARICSCLWMMLLISQAEA

SEQ ID NO: 10 - protein, 746 aa, Hepacivirus C
MSTNPKPQRKTKRNTNRRPQDVKFPGGGQIVGGVYLLPRRGPRLGVRATRKTSERSQPRGRRQPIPKARRPEGRSWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGIYHVTNDCPNSSIVYETADTILHSPGCVPCVREGNASKCWVALAPTVATRDGRLPTTQLRRHIDLLVGSATLCSALYVGDLCGSVFLVGQLFTFSPRRHWTTQDCNCSIYPGHITGHRMAWDMMMNWSPTTALVVAQLLRIPQAILDMIAGAHWGVLAGIAYFSMVGNWAKVLVVLLLFAGVDAHTRVTGGSAARATARLTTLFSPGAKQDIQLINTNGSWHINRTALNCNASLDTGWVAGLFYYHKFNSSGCPERMASCRPLADFDQGWGPISYANGSGPEHRPYCWHYPPKPCGIIPAKTVCGPVYCFTPSPVVVGTTDRSGAPTFNWGDNDTDVLVLNNTRPPLGNWFGCTWMNSSGYTKVCGAPPCIIGGVGNNTLHCPTDCFRKHPEATYSRCGSGPWITPRCLVHYPYRLWHYPCTVNYTLFKVRMYVGGVEHRLEAACNWTRGERCNLDDRDRSELSPLLLSTTQWQVLPCSFTTLPALTTGLIHLHQNIVDVQYLYGVGSSIVSWAIKWEYVILLFLLLADARVCSCLWMMLLISQVEA

SEQ ID NO: 11 - protein, 746 aa, Hepacivirus C
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASKCWVAVTPTVATRDGRLPATQLRRHIDLLVGSATLCSALYVGDLCGSIFLVGQLFTFSPRRHWTTQDCNCSIYPGHITGHRMAWDMMMNWSPTTALVVAQLLRVPQAILDMIAGAHWGVLAGIAYFSMVGNWAKVLVVLLLFAGVDAGTHVTGGSAAKDTSGFTSLFRIGARQNIQLINSNGSWHINRTALNCNESLDTGWVAGLLYYHKFNSSGCPERMASCRSLADFDQGWGPISYANGSGPEHRPYCWHYPPKPCGIVPAQSVCGPVYCFTPSPVVVGTTDKSGAPTYNWGCNETDVFVLNNTRPPLGNWFGCTWMNSSGFTKVCGAPPCVIGGVGNNTLHCPTDCFRKHPEATYSRCGSGPWITPRCLVHYAYRLWHYPCTVNYTLFKVRMYVGGVEHRLDVACNWTRGERCNLDDRDRSELSPLLLSTTQWQVLPCSFTTLPALSTGLIHLHQNIVDVQYLYGVGSSIVSWAIKWEYVILLFLLLADARICSCLWMMLLISQAEA

SEQ ID NO: 12 - protein, 746 aa, Hepacivirus C
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADSILHSPGCVPCVREGNASKCWVAVAPTVATRDGKLPATQLRRHIDLLVGSATLCSALYVGDLCGSVFLVSQLFTFSPRRHWTTQDCNCSIYPGHITGHRMAWDMMMNWSPTTALVVSQLLRVPQAILDMIAGAHWGVLAGIAYFSMVGNWAKVLVVLLLFAGVDAETHTTGGSAAYATSGFVGLFRQGAKQNIQLINTNGSWHVNRTALNCNASLDTGWVAGLFYYHKFNSSGCPERLASCKPLANFDQGWGSISYTNGSGPEHRPYCWHYPPKPCGIVPAQNVCGPVYCFTPSPVVVGTTDRLGAPTFNWGENESDVFVLNNTRPPSGNWFGCTWMNSSGFTKVCGAPPCNIGGVGNNTLHCPTDCFRKHPEATYSRCGSGPWVTPRCLVHYPYRLWHYPCTLNYSLFKVRMYVGGIEHRLEVACNWTRGERCNLDDRDRSELSPLLLTTTQWQVLPCSFTTLPALTTGLIHLHQNVVDVQYLYGVGSSIVSWAIKWEYVILLFLLLADARICSCLWMMLLISQAEA

SEQ ID NO: 13 - protein, 746 aa, Hepacivirus C
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYETADTILHSPGCVPCVREGNASKCWVAVAPTVATRDGRLPTTQLRRHIDLLVGSATLCSALYVGDLCGSVFLVGQLFTFSPRHHWTTQDCNCSIYPGHITGHRMAWDMMMNWSPTTALVVAQLLRVPQAILDMIAGAHWGVLAGIAYFSMVGNWAKVLVVLLLFAGVDATTHTTGGAVAHNTRMFTSIFSLGPRQEIQLVNTNGSWHINRTALNCNASLETGWIAGLLYANRFNSSGCPERMASCKPLADFDQGWGPISYANGSGPEHRPYCWHYPPKPCGIVPAQNVCGPVYCFTPSPVVVGTTDRLGTPTYDWGSNDTDVFVLNNTRPPAGNWFGCTWMNSSGYTKVCGAPPCVIGGVSNNTLHCPTDCFRKHPEATYSRCGSGPWITPRCLVHYAYRLWHYPCTVNYTLFKVRMYVGGVEHRLEVACNWTRGERCNLDDRDRSELSPLLLSTTQWQVLPCSFTTLPALTTGLIHLHQNIVDVQYLYGVGSSIVSWAVKWEYVILLFLLLADARICSCLWMMLLISQAEA

SEQ ID NO: 14 - protein, 746 aa, Hepacivirus C
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNASIVYEAADMIMHTPGCVPCVRENNSSRCWVALTPTLAARNASVPTTTIRRHVDLLVGAAALCSAMYVGDLCGSVFLVAQLFTFSPRRHETVQDCNCSIYPGHVTGHRMAWDMMMNWSPTAALVVSQLLRIPQAVVDMVAGAHWGVLAGLAYYSMVGNWAKVLIVMLLFAGVDGGTYVTGGTMAKNTLGITSLFSPGSSQKIQLVNTNGSWHINRTALNCNDSLNTGFLAALFYVHKFNSSGCPERMASCSPIDAFAQGWGPITYNESHSSDQRPYCWHYAPRPCGIVPAAQVCGPVYCFTPSPVVVGTTDRFGVPTYSWGENETDVLLLNNTRPPQGNWFGCTWMNSTGFTKTCGGPPCNIGGIGNKTLTCPTDCFRKHPEATYTKCGSGPWLTPRCLVHYPYRLWHYPCTVNFTIFKVRMYVGGVEHRLEAACNWTRGERCNLEDRDRSELSPLLLSTTEWQVLPCSFTTLPALSTGLIHLHQNVVDVQYLYGIGSAVVSFAIKWEYVLLLFLLLADARVCACLWMMLLIAQAEA

SEQ ID NO: 15 - protein, 746 aa, Hepacivirus C
MSTNPKPQRKTKRNTNRRPQDVKFPGGGQIVGGVYLLPRRGPRLGVRATRKTSERSQPRGRRQPIPKARQPEGRAWAQPGYPWPLYGNEGMGWAGWLLSPRGSRPSWGPTDPRRRSRNLGKVIDTLTCGFADLMGYIPLVGAPLGGVARALAHGVRVVEDGVNYATGNLPGCSFSIFLLALLSCLTIPASAYEVRNVSGIYHVTNDCSNSSIVYEAADMIMHTPGCVPCVREGNSSRCWVALTPTLAARNASVPTTAIRRHVDLLVGAAAFCSAMYVGDLCGSVFLVSQLFTFSPRRHETIQDCNCSIYPGHVSGHRMAWDMMMNWSPTTALVVSQLLRIPQAIVDMVAGAHWGVLAGLAYYSMVGNWAKVLIVMLLFAGVDGETRVTGGQIARNAYSLTTLFSSGSAQNIQLINTNGSWHINRTALNCNDSLNTGFLAALFYTHKFNASGCPERLASCRPIDKFDQGWGPITYAEQGGQDQRPYCWHYAPKPCGIVSASKVCGPVYCFTPSPVVVGTTDRFGVPTYSWGENETDVLLLNNTRPPQGNWFGCTWMNGTGFTKTCGGPPCNIGGGGNNTLTCPTDCFRKHPAATYTKCGSGPWLTPRCLVDYPYRLWHYPCTANFTIFKVRMYVGGVEHRLDAACNWTRGERCNLEDRDRLELSPLLLSTTEWQVLPCSFTTLPALSTGLIHLHQNIVDVQYLYGIGSAVVSFAIKWDYIVILFLLLADARVCACLWMMLLIAQAEA

SEQ ID NO: 16 - protein, 747 aa, Hepacivirus C
MSTNPKPQRKTKRNTNRRPQDVKFPGGGQIVGGVYLLPRRGPRLGVRAIRKTSERSQPRGRRQPIPKARRPEGRAWAQPGYPWPLYGNEGMGWAGWLLSPRGSRPSWGPTDPRRRSRNLGKVIDTLTCGLADLMGYIPLVGGPLGGAARALAHGVRVLEDGVNYATGNLPGCSFSIFLLALLSCLTVPASAHEVRNASGVYHVTNDCSNSSIVFEAADLIMHTPGCVPCVREGNSSRCWVALTPTLAARNATIPTTTIRHHVDLLVGAAALCSAMYVGDLCGSVFLVSQLFTFSPRRHATLQDCNCSIYPGHASGHRMAWDMMMNWSPTTALVVSQLLRIPQAVIDMVAGAHWGVLAGLAYYSMAGNWAKVLIVMLLFAGVDGHTLTTGGHAARLTSGFAGLFTPGPSQRIQLINTNGSWHINRTALNCNDSLQTGFLAALFYAHRFNSSGCPERMASCRSIDKFDQGWGPITYAEPTKDPDQRPYCWHYPPQQCGIVPASQVCGPVYCFTPSPVVVGTTDRLGNPTYSWGENDTDVLLLNNTRPPQGNWFGCTWMNSTGFTKTCGAPPCNIGGVGNNTLTCPTDCFRKHPEATYSKCGSGPWLTPRCMVDYPYRLWHYPCTVNFSIFKVRMYVGGVEHRLNAACNWTRGERCNLDDRDRSELSPLLLSTTEWQVLPCSFTTLPALSTGLIHLHQNIVDVQYLYGIGSAVVSFAIKWEYVVLLFLLLADARVCACLWMMLLIAQAEA

SEQ ID NO: 17 - protein, 746 aa, Hepacivirus C
MSTNPKPQRKTKRNTNRRPQDVKFPGGGQIVGGVYLLPRRGPRLGVRATRKTSERSQPRGRRQPIPKARHPEGRTWAQPGYPWPLYGNEGLGWAGWLLSPRGSRPSWGPTDPRRRSRNLGKVIDTLTCGFADLMGYIPLVGAPLGGVARALAHGVRVLEDGVNYATGNLPGCSFSIFLLALLSCLTIPASAYEVRNVSGVYHVTNDCSNSSIVYEAADVIMHTPGCVPCVQDGNTSRCWVALTPTLAARNASVPVTAIRRHVDLLVGTAAFCSAMYVGDLCGSVFPVSQLFTFSPRRHQTVQDCNCSIYPGHISGHRMAWDMMMNWSPTAALVVSQLLRIPQAIVDMVAGAHWGVLAGLAYYSMVGNWAKVMIVLLLFAGVDGTTHTTGGAAARATQGFTSFFSLGPSQKIQLINTNGSWHINRTALNCNDSLQTGFLAALFYTYRFNASGCPERMASCRPIDKFDQGWGPITYAEPDSSDQRPYCWHYAPRPCGIVPASQVCGPVYCFTPSPVVVGTTDRFGVPTYTWGENETDVLLLNNTRPPLGNWFGCTWMNSTGFTKTCGGPPCNIGGAGNTTLTCPTDCFRKHPEATYTKCGSGPWLTPRCLVDYPYRLWHYPCAVNFTIFKVRMYVGGVEHRLNAACNWTRGERCDLEDRDRSELSPLLLSTTEWQVLPCSFTTLPALSTGLIHLHQNIVDVQYLYGIGSAVIPFAIKWEYVLLLFLLLADARVCACLWMMLLIAQAEA

SEQ ID NO: 18 - protein, 746 aa, Hepacivirus C
MSTNPKPQRKTKRNTNRRPQDVKFPGGGQIVGGVYLLPRRGPRLGVRATRKTSERSQPRGRRQPIPKARRPEGRTWAQPGYPWPLYGNEGMGWAGWLLSPRGSRPNWGPTDPRRRSRNLGKVIDTLTCGFADLMGYIPLVGAPLGGVARALAHGVRVLEDGVNYATGNLPGCSFSIFLLALLSCLTTPASAYEVRNVSGIYHVTNDCSNSSIVYEAADIIMHTPGCVPCVREKNISRCWVALTPTLAARNISVPTATIRRHVDLLVGTAAFCSAMYVGDLCGSVFLVSQLFTFSPRWHETVQDCNCSLYPGHVSGHRMAWDMMMNWSPTAALVVSQLLRIPQAVVDMVAGAHWGVLAGLAYYSMIGNWAKVLIVMLLFAGADGTTHVTGGVQAHGAYGLASLFNVGPHQKIQLVNTNGSWHINRTALNCNDTLQTGFLAALFYKHRFNASGCPERMASCRPIDKFAQGWGPITYAEPDRLDQRPYCWHYPPRPCGIVPALEVCGPVYCFTPSPVVVGTTDRFGVPTYSWGENETDVLLLNNTRPPQGNWFGCTWMNSTGYTKTCGGPPCNIGGVGNNTLTCPTDCFRKHPEATYTKCGSGPWLTPRCLVHYPYRLWHYPCTVNFTIFKVRMYVGGIEHRLDAACNWTRGERCDLEDRDRSELSPLLLSTTEWQILPCSFTTLPALSTGLIHLHRNIVDVQYLYGIGSAVVSFAIKWEYVLLLFLLLADARVCACLWMMLLIAQAEA

SEQ ID NO: 19 - protein, 746 aa, Hepacivirus C
MSTNPKPQRKTKRNTNRRPQDVKFPGGGQIVGGVYLLPRRGPRLGVRATRKTSERSQPRGRRQPIPKARRPEGRTWAQPGYPWPLYGNEGLGWAGWLLSPRGSRPSWGPNDPRRRSRNLGKVIDTLTCGFADLMGYIPLVGAPLGGAARALAHGVRVLEDGVNYATGNLPGCSFSIFLLALLSCLTIPASAYEVRNVSGIYHVTNDCSNSSIVYEAADVIMHAPGCVPCVRENNSSRCWVALTPTLAARNASVPTTTLRRHVDLLVGTAAFCSAMYVGDLCGSVFLISQLFTFSPRRHETVQDCNCSIYPGHVSGHRMAWDMMMNWSPTAALVVSQLLRIPQAVMDMVAGAHWGVLAGLAYYSMVGNWAKVLIVMLLFAGVDGHTRVTGGVQGHVTSTLTSLFRPGASQKIQLVNTNGSWHINRTALNCNDSLKTGFLAALFYTHKFNASGCPERMASCRSIDKFDQGWGPITYAQPDNSDQRPYCWHYAPRQCGIVPASQVCGPVYCFTPSPVVVGTTDRFGAPTYNWGDNETDVLLLNNTRPPHGNWFGCTWMNSTGFTKTCGGPPCNIRGVGNNTLTCPTDCFRKHPDATYTKCGSGPWLTPRCLVDYPYRLWHYPCTVNFTIFKVRMYVGGVEHRLDAACNWTRGERCDLEDRDRAELSPLLLSTTEWQILPCSYTTLPALSTGLIHLHQNIVDIQYLYGIGSAVVSIAIKWEYVVLLFLLLADARVCACLWMMLLIAQAEA

SEQ ID NO: 20 - protein, 746 aa, Hepacivirus C
MSTNPKPQRKTKRNTNRRPQDVKFPGGGQIVGGVYLLPRRGPRLGVRATRKTSERSQPRGRRQPIPKARRPEGRTWAQPGYPWPLYGDEGMGWAGWLLSPRGSRPNWGPTDPRRRSRNLGKVIDTLTCGFADLMGYIPLVGAPLGGVARALAHGVRVLEDGVNYATGNLPGCSFSIFLLALLSCLTTPASAYEVRNVSGIYHVTNDCSNSSIVYEAADIIMHTPGCVPCVREKNTSRCWVALTPTLAARNISVPTTTIRRHVDLLVGTAAFCSAMYVGDLCGSVFLVSQLFTFSPRRHETVQDCNCSLYPGHVSGHRMAWDMMMNWSPTAALVVSQLLRIPQAIVDMVAGAHWGVLAGLAYYSMVGNWAKVLIVMLLFAGADGTTHVTGGVQAHGAYGLASLFNVGPHQKIQLVNTNGSWHINRTALNCNDTLQTGFLAALFYKHRFNASGCPERMASCRPIDKFAQGWGPITYAEPDRLDQRPYCWHYPPRPCGIVPALEVCGPVYCFTPSPVVVGTTDRFGVPTYSWGENETDVLLLNNTRPPQGNWFGCTWMNNTGFTKTCGGPPCNIGGVGNNTLTCPTDCFRKHPEATYTKCGSGPWLTPRCLVDYPYRLWHYPCTVNFTVFKVRMYVGGIEHRLDAACNWTRGERCDLEDRDRSELSPLLLSTTEWQILPCSFTTLPALSTGLIHLHRNIVDVQYLYGIGSAVVSFAIKWEYILLLFLLLADARVCACLWMMLLIAQAEA

SEQ ID NO: 21 - protein, 746 aa, Hepacivirus C
MSTNPKPQRKTKRNTNRRPQDVKFPGGGQIVGGVYLLPRRGPRLGVRATRKTSERSQPRGRRQPIPKARQPEGRAWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNSSIVYEAADMIMHTPGCVPCVREDNSSRCWVALTPTLAARNSSVPTTTIRRHVDLLVGAAAFCSAMYVGDLCGSVFLISQLFTFSPRRYETVQDCNCSLYPGHVSGHRMAWDMMMNWSPTTALVVSQLLRIPQAVVDMVAGAHWGVLAGLAYYSMVGNWAKVLIVMLLFAGVDGNTRVSGGEAAKNTMGFASLFVSGPSQKIQLINTNGSWHINRTALNCDDSLHTGFLAALFYAHKFNSSGCSGRMASCRPIDEFAQGWGPITHGVPDNLDQRPYCWHYAPRPCGIVPASQVCGPVYCFTPSPVVVGTTDRFGAPTYSWGENETDVLLLNNTRPPQGNWFGCTWMNSTGFTKTCGGPPCNIGGVGNNTLTCPTDCFRKHPEATYTKCGSGPWLTPRCMVDYPYRLWHYPCTVNFTIFKVRMYVGGVEHRLDAACNWTRGERCNVEDRDRSELSPLLLSTTEWQILPCSFTTLPALSTGLIHLHQNIVDVQYLYGVGSVVVSVVIRWEYVVLLFLLLADARVCACLWMMLLIAQAEA

SEQ ID NO: 22 - protein, 746 aa, Hepacivirus C
MSTNPKPQRKTKRNTNRRPQDVKFPGGGQIVGGVYLLPRRGPRLGVRATRKTSERSQPRGRRQPIPKARQPEGRAWAQPGYPWPLYGNEGMGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGMYHVTNDCSNSSIVYEAADMILHAPGCVPCVRENNSSRCWVALTPTLAARNASVPTTAIRRHVDLLVGAAAFCSAMYVGDLCGSVLLVSQIFTFSPRRHETMQDCNCSIYPGHITGHRMAWDMMMNWSPTTALVVSQLLRIPQAIVDMVAGAHWGVLAGLAYYSMVGNWAKVLIVMLLFAGVDGGTYVTGGEAGRRTSGFASIFTPGASQNIQLINTNGSWHINRTALNCNDSLHTGFIAALFYHHKFNASGCPERMASCRPIGEFAQGWGPISYTEPPSSDQRPYCWHYPPRPCGIVPASQVCGPVYCFTPSPVVVGTTDRLGAPTYNWGDNDTDVLLLNNTRPPQGNWFGCTWMNGTGFTKTCGGPPCNIGGVGNNTLTCPTDCFRKHPEATYTKCGSGPWLTPRCIVDYPYRLWHYPCTVNFTITKIRMYVGGVEHRLTAACNWTRGERCDLEDRDRSELSPLLLSTTEWQIMPCSFTTLPALSTGLIHLHQNIVDIQYLYGIGSAAVSFAIRWEYVLLLFLLLADARVCACLWMMLLIAQAEA

SEQ ID NO: 23 - protein, 746 aa, Hepacivirus C
MSTNPKPQRKTKRNTNRRPQDVKFPGGGQIVGGVYLLPRRGPRLGVRATRKTSERSQPRGRRQPIPKARRPEGRAWAQPGYPWPLYGNEGLGWAGWLLSPRGSRPSWGPTDPRRRSRNLGKVIDTLTCGFADLMGYIPLVGAPLGGTARALAHGVRVLEDGVNYATGNLPGCSFSIFLLALLSCLTIPASAYEVRNVSGVYHVTNDCSNSSIVYETADMIMHTPGCVPCVREDNFTRCWVALTPTLAARNGSVPTTAIRRHVDLLVGAAAFCSAMYVGDLCGSVFLVSQLFTFSPRWHETVQECNCSIYPGHVTGHRMAWDMMMNWSPTTALVVSQLLRIPQAVVDMVAGAHWGVLAGLAYYSMVGNWAKVLIVTLLFAGVDGNTHTIGGKQAQATGGFVAWLARGPSQEIQLINTNGSWHINRTALNCNDSLKTGFIAALFYAHRFNSSGCPERMASCRPIDKFAQGWGPITYAKPDSLDQRPYCWHYAPQPCGIVPASEVCGPVYCFTPSPVVVGTTDRSGVPTYRWGENETDVLLLNNTRPPQGNWFGCTWMNATGFTKTCGGPPCKIGGLGNNTLTCPTDCFRKHPEATYTKCGSGPWLTPRCIVDYPYRLWHYPCTVNFTIFKVRMYVGGIEHRLSAACNWTRGERCDLEDRDRSELSPLLLSTTEWQILPCSFTTLPALSTGLIHLHQNTVDVQYLYGVGSVLVSFAIKWEYILLFFLLLADARVCACLWMMLLIAQAEA

SEQ ID NO: 24 - protein, 746 aa, Hepacivirus C
MSTNPKPQRKTKRNTNRRPQNVKFPGGGQIVGGVCLLPRRGPRVGVRATRKTSERSQPRGRRQPIPKARRPEGRSWAQPGYPWPLYGNEGCGWAGWLLSPRGSRPSRGPSDPRRRSRNLGKVIDTLTCGFADLMGYIPLVGAPLGGAARALAHGVRVLEDGVNYATGNLPGCSFSIFLLALLSCLTVPASAVEVRNSSGIYHVTNDCPNASVVYETDSLIIHLPGCVPCVREGNASRCWVSLSPTVAAKDPGVPVNEIRRHVDLIVGAAAFCSAMYVGDLCGSIFLVGQLFTLSPRRHWTTQDCNCSIYPGHVTGHRMAWDMMMNWSPTGALVVAQLLRIPQAVLDMIAGAHWGVLAGPAYYSMVGNWAKVLVVLLLFAGVDATTQVTGGTAGRNAYRLASLFSTGPSQNIQLINSNGSWHINRTALNCNDSLHTGWVAALFYSHKFNSSGRPERMASCRPLTAFDQGWGPITYGGKASNDQRPYCWHYAPRPCGIVPAKEVCGPVYCFTPSPVVVGTTDKYGVPTYTWGENETDVLLLNNSRPPIGNWFGCTWMNSTGFTKTCGAPACNVGGSETNTLSCPTDCFRRHPDATYAKCGSGPWLNPRCMVDYPYRLWHYPCTVNYTIFKIRMFVGGIEHRLTAACNWTRGERCDLDDRDRAELSPLLLSTTQWQVLPCSFTTLPALSTGLIHLHQNIVDVQYLYGLSSVVTSWAIRWEYVVLLFLLLADARICACLWMMLLISQVEA

SEQ ID NO: 25 - protein, 746 aa, Hepacivirus C
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTYGFADLMGYIPLVGAPLGGAARALAHGVRVLEDGVNYATGNLPGCSFSIFLLALLSCLTVPASAVGVRNSSGVYHVTNDCPNASVVYETDSLIIHLPGCVPCVREGNGSRCWVSLSPTVAAKDPGVPVNEIRRHVDLIAGAAAFCSAMYVGHLCGSIFLVGQLFTLSPRRHWTTQDCNCSIYPGHVTGHRMAWDMMMNWSPTTALVVAQLLRIPQAILDMIAGAHWGVLAGIAYFSMVGNWTKVLVVLLLFAGVDATTIVSGGSAGRSTAGLVGLFSPGARQNIQLINTNGSWHINRTALNCNDTLQTGWVAGLFYTNKFNSSGCPERLASCRPLADFDQGWGPISYTNGSGPDQRPYCWHYPPKPCGIVPAESVCGPVYCFTPSPVVVGTTDRSGAPTYNWGENETDVFVLNNTRPRLGNWFGGTWMNSTGFTKVCGAPPCAIGGVGNNTLYCPTDCFRKHPEATYSRCGSGPWITPRCLIHYPYRLWHYPCTINYTIFKIRMFVGGVEHRLDAACNWTRGERCDLDDRDRAELSPLLLSTTQWQVLPCSFTTLPALSTGLIHLHQNIVDVQYLYGLSSAVTSWVIKWEYVVLLFLLLADARICACLWMMLLISQVEA

SEQ ID NO: 26 - protein, 746 aa, Hepacivirus C
MSTNPKPQRKTKRNTNRRPQDVKFPGGGQIVGGVYLLPRRGPRVGVRATRKTSERSQPRGRRQPIPKARRPEGRSWAQPGYPWPLYGNEGCGWAGWLLSPRGSRPSWGPSDPRRRSRNLGKVIDTLTCGFADLMGYIPLVGAPLGGAARALAHGVRVLEDGVNYATGNLPGCSFSIFLLALLSCLTVPASAVGVRNSSGVYHVTNDCPNASVVYETENLIMHLPGCVPYVREGNASRCWVSLSPTVAARDSRVPVSEVRRRVDSIVGAAAFCSAMYVGDLCGSIFLVGQIFTFSPRHHWTTQDCNCSIYPGHVTGHRMAWDMMMNWSPTGALVVAQLLRIPQAIVDMIAGAHWGVLAGLAYYSMVGNWAKVVVVLLLFAGVDAETRVTGGAAGHTAFGFASFLAPGAKQKIQLINTNGSWHINRTALNCNESLDTGWLAGLLYYHKFNSSGCPERMASCQPLTAFDQGWGPITHEGNASDDQRPYCWHYALRPCGIVPAKKVCGPVYCFTPSPVVVGTTDRAGVPTYRWGANETDVLLLNNSRPPMGNWFGCTWMNSSGFTKTCGAPACNIGGSGNNTLLCPTDCFRKHPDATYSRCGSGPWLTPRCLVDYPYRLWHYPCTVNYTIFKIRMFVGGVEHRLDAACNWTRGERCDLDDRDRAELSPLLLSTTQWQVLPCSFTTLPALSTGLIHLHQNIVDVQYLYGLSSAVTSWVIKWEYVVLLFLLLADARICACLWMMLLISQVEA

SEQ ID NO: 27 - protein, 750 aa, Hepacivirus C
  VARIANT 297: note = X can be any naturally occurring amino acid
  VARIANT 372: note = X can be any naturally occurring amino acid
  VARIANT 392: note = X can be any naturally occurring amino acid
  VARIANT 394: note = X can be any naturally occurring amino acid
  VARIANT 405: note = X can be any naturally occurring amino acid
  VARIANT 446: note = X can be any naturally occurring amino acid
  VARIANT 548: note = X can be any naturally occurring amino acid
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITVPVSAVEVKNISTSYMVTNDCSNDSITWQLQAAVLHVPGCVPCENDGNTSRCWIPVSPNVAVQQPGALTQGLRTHIDMVVMSATLCSALYVGDLCGGVMLAAQMFIVSPQXHWFVQECNCSIYPGTITGHRMAWDMMMNWSPTATMILAYAMRVPEVIIDIIGGAHWGVMFGLAYFSMQGAWAKVXVILLLAAGVDARTHTVGGSXGXTTSGLAGLFSXGPKQNIQLINTNGSWHINRTALNCNDSLQTGFLASLFYTHXFNSSGCPERLSACRGIEAFRIGWGTLQYEDNVTNPEDMRPYCWHYPPKQCGIVPARSVCGPVYCFTPSPVVVGTTDRLGVPTYTWGENETDVFLLNSTRPPXGSWFGCTWMNSTGFTKTCGAPPCRIRADFNASTDLLCPTDCFRKHPDATYIKCGSGPWLTPRCLVDYPYRLWHYPCTVNYTIFKIRMYVGGVEHRLTAACNFTRGDRCNLEDRDRSQLSPLLHSTTEWAILPCSYSDLPALSTGLLHLHQNIVDVQYMYGLSPALTKYIVRWEWVVLLFLLLADARVCACLWMLILLGQAEA

SEQ ID NO: 28 - protein, 750 aa, Hepacivirus C
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPNDPRHRSRNVGKVIDTLTCGFADLMGYIPVVGAPLGGVARALAHGVRVLEDGVNFATGNLPGCSFSIFLLALLSCITTPVSAAEVKNISTGYMVTNDCTNDSITWQLQAAVLHVPGCVPCEKVGNTSRCWIPVSPNVAVQQPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIIGGAHWGVMFGLAYFSMQGAWAKVVVILLLAAGVDAQTHTVGGSTAHNARTLTGMFSLGARQKIQLINTNGSWHINRTALNCNDSLHTGFLASLFYTHSFNSSGCPERMSACRSIEAFRVGWGALQYEDNVTNPEDMRPYCWHYPPRQCGVVSASSVCGPVYCFTPSPVVVGTTDRLGAPTYTWGENETDVFLLNSTRPPQGSWFGCTWMNSTGYTKTCGAPPCRIRADFNASMDLLCPTDCFRKHPDTTYIKCGSGPWLTPRCLIDYPYRLWHYPCTVNYTIFKIRMYVGGVEHRLTAACNFTRGDRCNLEDRDRSQLSPLLHSTTEWAILPCTYSDLPALSTGLLHLHQNIVDVQFMYGLSPALTKYIVRWEWVVLLFLLLADARVCACLWMLILLGQAEA

SEQ ID NO: 29 - protein, 750 aa, Hepacivirus C
MSTNPKPQRKTKRNTNRRPEDVKFPGGGQIVGGVYLLPRRGPRLGVRTTRKTSERSQPRGRRQPIPKDRRSTGKAWGKPGRPWPLYGNEGLGWAGWLLSPRGSRPSWGPTDPRHRSRNVGKVIDTLTCGFADLMGYIPVVGAPLSGAARAVAHGVRVLEDGVNYATGNLPGFPFSIFLLALLSCITVPVSAAQVKNTSSSYMVTNDCSNDSITWQLEAAVLHVPGCVPCERVGNTSRCWVPVSPNMAVRQPGALTQGLRTHIDMVVMSATFCSALYVGDLCGGVMLAAQVFIVSPQYHWFVQECNCSIYPGTITGHRMAWDMMMNWSPTATMILAYVMRVPEVIIDIVSGAHWGVMFGLAYFSMQGAWAKVIVILLLAAGVDAGTTTVGGAVARSTNVIAGVFSHGPQQNIQLINTNGSWHINRTALNCNDSLNTGFLAALFYTNRFNSSGCPGRLSACRNIEAFRIGWGTLQYEDNVTNPEDMRPYCWHYPPKPCGVVPARSVCGPVYCFTPSPVVVGTTDRRGVPTYTWGENETDVFLLNSTRPPQGSWFGCTWMNSTGFTKTCGAPPCRTRADFNASTDLLCPTDCFRKHPDATYIKCGSGPWLTPKCLVHYPYRLWHYPCTVNFTIFKIRMYVGGVEHRLTAACNFTRGDRCDLEDRDRSQLSPLLHSTTEWAILPCTYSDLPALSTGLLHLHQNIVDVQYMYGLSPAITKYVVRWEWVVLLFLLLADARVCACLWMLILLGQAEA

SEQ ID NO: 30 - protein, 749 aa, Hepacivirus C
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITTPVSAAQVKNTSDIYMVTNDCSNDSITWQLQAAALHVPGCVPCERVGNTSRCWIPVSPNVAVRQPGALTQGLRTHIDMVVMSATLCSALYVGDLCGGVMLAAQMFIISPQHHWFVQECNCSIYPGTITGHRMAWDMMMNWSPTTTMILAYVMRVPEVIIDIIGGAHWGVMFGLAYFSMQGAWAKVVVILLLAAGVDAHTRTGSSVGYATSGIVGLFTSGPKQNIQLINTNGSWHINRTALNCNDSLNTGFIVSLFYARNFNSTGCPERLSACRGIEGFRIGWGTLQYEDNVTNPEDMRPYCWHYPPKQCGIVPAGSVCGPVYCFTPSPVVVGTTDRLGVPTYTWGENETDVFLLNSTRPPVGSWFGCTWMNSTGFTKTCGAPPCRIRADFNASTDLLCPTDCFRKHPEATYIKCGSGPWLTPRCLVDYPYRLWHYPCTVNYSIFKIRMYVGGVEHRLTAACNFTRGDRCNLEDRDRSQLTPLLHSTTEWAILPCTYSDLPALSTGLLHLHQNIVDVQYMYGLSPALTKYVVRWEWVVLLFLLLADARVCACVWMLILLGQAEA

SEQ ID NO: 31 - protein, 749 aa, Hepacivirus C
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCVTVPVSAVQVKNTSETYMVTNDCSNDSITWQLQAAVLHVPGCVPCERTGNTSRCWIPVSPNVAVQQPGALTQGLRTHIDMVVMSATLCSALYVGDLCGGVVLAAQLFIVSPRRHWFVQECNCSIYPGAITGHRMAWDMMMNWSPTTTMVLAYAMRVPEVIIDIISGAHWGVMFGLAYFSMQGAWAKVAVILLLTAGVEARTHTTGSVAGRTTSGFAGIFTSGPKQNIQLINTNGSWHINRTALNCNDSLNTGFMAALFYTKNFNSSGCPERLSCRNIEAFRIGWGTLQYEDDVTNPEDMRPYCWHYPPKQCGIFPAGSVCGPVYCFTPSPVVVGTTNKLGVPTYTWGENETDVFILNSTRPPRGSWFGCTWMNSTGFTKTCGAPPCRIRADFNASTDLLCPTDCFRKHPEATYIKCGSGPWLTPRCLVDYPYRLWHYPCTVNYSIFKIRMYVGGVEHRLTAACNFSRGDRCNLEDRDRSQLTPLLHSTTEWAILPCSYSDLPALSTGLLHLHQNIVDVQYMYGLTPALTKYVVRWEWVVLLFLLLADARVCACLWMLILLGQAEA

SEQ ID NO: 32 - protein, 751 aa, Hepacivirus C
MSTNPKPQRKTQRNTNRRPQDVKFPGGGQIVGGVYLLPRRGPRLGVRTARKTSERSQPRGRRQPIPKDRRSTGKSWGRPGYPWPLYGNEGLGWAGWLLSPRGSRPSWGPTDPRHRSRNVGKVIDTLTCGFADLMGYIPVVGAPLGGVARALAHGVRVLEDGVNYATGNLPGCSFSIFLLALLSCITIPASAVEVKNTSTGYMVTNDCANSSITWQLHAAVLHVPGCVPCERVDNNTSRCWIPVSPNIAVQRPGALTQGLRSHIDIVVMSATLCSALYVGDLCGGVMLAAQMFVVSPEHHWFVQECNCSIYPGTITGHRMAWDMMMNWSPTATMILAYAMRVPEVIIDIIGGAHWGVMFGLAYFSMQGAWAKVVVILLLAAGVDAYTHTVGGAAASTANSIAGLLSRGPRQNLQLINSNGSWHINRTALNCHDSLQTGFITALFYARHFNSSGCPERLAACRNIEAFRVGWGALQYEDNVTNPEDMRPYCWHYPPKQCGIVPARSVCGPVYCFTPSPVVVGTTDKLGVPTYTWGENETDVFLLNSTRPPQGPWFGCTWMNSTGFTKTCGAPPCRTRADFNASTDLLCPTDCFRKHPDATYNKCGSGPWLTPRCLIDYPYRLWHYPCTVNYTTFKIRMYVGGVEHRLMAACNFTRGDSCDLSQRDRGQLSPLLHSTTEWAILPCSFSDLPALSTGLLHLHQNIVDVQYMYGLSPALTKYIVRWEWVVLLFLLLADARVCACIWMLILLGQAEA

SEQ ID NO: 33 - skipped

SEQ ID NO: 34 - protein, 750 aa, Hepacivirus C
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPNDPRHRSRNVGKVIDTLTCGFADLMGYIPVVGAPLGGVARALAHGVRVLEDGVNFATGNLPGCSFSIFLLALLSCITTPVSAAEVKNISTGYMVTNDCTNDSITWQLQAAVLHVPGCVPCEKVGNASQCWIPVSPNVAVQRPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIISGAHWGVMFGLAYFSMQGAWAKVVVILLLAAGVDARTHTVGGSAAQTTGRLTSLFDMGPRQKIQLVNTNGSWHINRTALNCNDSLHTGFIASLFYTHSFNSSGCPERMSACRSIEAFRVGWGALQYEDNVTNPEDMRPYCWHYPPRQCGVVSAKTVCGPVYCFTPSPVVVGTTDRLGAPTYTWGENETDVFLLNSTRPPLGSWFGCTWMNSSGYTKTCGAPPCRTRADFNASTDLLCPTDCFRKHPDTTYLKCGSGPWLTPRCLIDYPYRLWHYPCTVNYTIFKIRMYVGGVEHRLTAACNFTRGDRCNLEDRDRSQLSPLLHSTTEWAILPCSYSDLPALSTGLLHLHQNIVDVQFMYGLSPALTKYIVRWEWVILLFLLLADARVCACLWMLILLGQAEA

SEQ ID NO: 35 - protein, 750 aa, Hepacivirus C
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPTDPRHRSRNLGRVIDTITCGFADLMGYIPVVGAPVGGVARALAHGVRVLEDGINYATGNLPGCSFSIFLLALLSCVTVPVSAVEVRNISSSYYATNDCSNNSITWQLTDAVLHLPGCVPCENDNGTLHCWIQVTPNVAVKHRGALTRSLRTHVDMIVMAATACSALYVGDVCGAVMILSQAFMVSPQRHNFTQECNCSIYQGHITGHRMAWDMMLSWSPTLTMILAYAARVPELVLEIIFGGHWGVVFGLAYFSMQGAWAKVIAILLLVAGVDATTYSSGQEAGRTVAGFAGLFTTGAKQNLYLINTNGSWHINRTALNCNDSLQTGFLASLFYTHKFNSSGCPERLSSCRGLDDFRIGWGTLEYETNVTNDGDMRPYCWHYPPRPCGIVPARTVCGPVYCFTPSPVVVGTTDKQGVPTYTWGENETDVFLLNSTRPPRGAWFGCTWMNGTGFTKTCGAPPCRIRKDYNSTIDLLCPTDCFRKHPDATYLKCGAGPWLTPRCLVDYPYRLWHYPCTVNFTIFKARMYVGGVEHRFSAACNFTRGDRCRLEDRDRGQQSPLLHSTTEWAVLPCSFSDLPALSTGLLHLHQNIVDVQYLYGLSPALTRYIVKWEWVILLFLLLADARICACLWMLIILGQAEA

SEQ ID NO: 36 - protein, 750 aa, Hepacivirus C
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPHWGPTDPRHRSRNLGKVIDTITCGFADLMGYIPVIGAPVGGVARALAHGVRVLEDGVNYATGNLPGCSFSIFLLALLSCVTVPVSAVEVRNISSSYYATNDCSNNSITWQLENAVLHLPGCVPCENDNGTLRCWTQVTPNVAVKHRGALTQNLRTHVDVIVVAATVCSALYVGDVCGAVMIASQALIVSPARHNFTQECNCSIYQGRITGHHMAWDMMLNWSPTITMILAYAARIPELVLEVIFGGHWGVMFGLAYFSMQGAWAKVIVILLLVAGVDARHHTTGLQAGKTLARVTSLFSIGAKQNIQLINTNGSWHINRTALNCNDSLQTGFIASLFYVNNINSSGCPERMSSCRELDDFRIGWGTLEYETNVTNDEDMRPYCWHYPPKPCGIVPARTVCGPVYCFTPSPIVVGTTDKQGVPTYSWGENETDVFLLNSTRPPRGSWFGCTWMNGTGFTKTCGAPPCRIRRDYNSTLDLLCPTDCFRKHPDTTYLKCGSGPWLTPKCLVEYPYRLWHYPCTVNFTIFKVRMYVGAVEHRFSAACNFTRGDRCRLEDRDRGQQSPLLHSTTEWAVLPCSFSDLPALSTGLLHLHQNIVDVQYLYGLSPAITRYIVKWEWVVLLFLLLADARVCACLWMLIILGQAEA

SEQ ID NO: 37 - protein, 750 aa, Hepacivirus C
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPTDPRHRSRNLGRVIDTITCGFADLMGYIPVVGAPVGGVARALAHGVRVLEDGINYATGNLPGCSFSIFLLALLSCCTVPVSAVEVRNISTSYYATNDCSNTSITWQLTNAVLHLPGCVPCENDNGTLRCWIQVTPNVAVKHRGALTHNLRTHVDVIVMAATVCSALYVGDICGAVMIVSQAFIISPERHNFTQECNCSMYQGHITGHRMAWDMMLNWSPTLTMILAYAARVPELVLEVIFGGHWGVVFGLAYFSMQGAWAKVIAILLLVAGVDANTYSSGVTVGHTTSTFANIFSVGPSQKINLINTNGSWHINRTALNCNDSLQTGFLASLFYVRNFNSSGCRERLSSCRRLDDFRIGWGTLEYETNVTNDEDMRPYCWHYPPKPCGIVSARTVCGPVYCFTPSPVVVGTTDRQGVPTYSWGENETDVFLLNSTRPPRGAWFGCTWMNGTGFTKTCGAPPCRIRRDYNSTLDLLCPTDCFRKHPDTTYLKCGAGPWLTPKCLVDYPYRLWHYPCTVNFTIFKVRMYVGGVEHRLSAACNFTRGDRCGLEDRDRGQQSPLLHSTTEWAVLPCSFSDLPALSTGLLHLHQNIVDVQYLYGLSPAITRYIVKWEWVVLLFLLLADARVCACLWMLIILGQAEA

SEQ ID NO: 38 - protein, 750 aa, Hepacivirus C
MSTNPKPQRKTKRNTNRRPQDVKFPGGGQIVGGVYLLPRRGPRLGVRATRKTSERSQPRGRRQPIPKDRRSTGKSWGKPGYPWPLYGNEGCGWAGWLLSPRGSRPTWGPSDPRHRSRNLGRVIDTITCGFADLMGYIPVVGAPVGGVARALAHGVRVLEDGINYATRNLPGCSFSIFLLALLSCVTVPVSSVEIRNISTSYYATNDCSNNSITWQLTNAVLHLPGCVPCENDNGTLRCWIQVTPNVAVKHRGALTHNLRAHVDVIVMAATVCSALYVGDVCGAVMIVSQALIVSPERHNFTQECNCSIYQGHITGQRMAWDMMLNWSPTLTMILAYAARVPELVLEIVFGGHWGVVFGLAYFSMQGAWAKVIAILLLVAGVDATTYSTGATVGRTVGSFAGLFKLGAQQNVQLINTNGSWHINRTALNCNDSLHTGFMAALFYANKFNSSGCPERLSSCRGLDDFRIGWGTLEYETNVTNVEDMRPYCWHYPPKPCGIVPAQSVCGPVYCFTPSPVVVGTTDRQGVPTYNWGDNETDVFLLNSTRPPRGAWFGCTWMNGTGFTKTCGAPPCRIRKDFNSTLDLLCPTDCFRKHPDATYVKCGAGPWLTPRCLIDYPYRLWHYPCTVNFTIFKVRMYVGGVEHRFSAACNFTRGDRCRLEDRDRGQQSPLLHSTTEWAVLPCSFSDLPALSTGLLHLHQNIVDVQYLYGLSPAVTKYIVKWEWVVLLFLLLADARICACLWMLIILGQAEA

SEQ ID NO: 39 - protein, 750 aa, Hepacivirus C
  VARIANT 203: note = X can be any naturally occurring amino acid
  VARIANT 295: note = X can be any naturally occurring amino acid
  VARIANT 343: note = X can be any naturally occurring amino acid
  VARIANT 401: note = X can be any naturally occurring amino acid
  VARIANT 426: note = X can be any naturally occurring amino acid
  VARIANT 626: note = X can be any naturally occurring amino acid
MSTDPKPQRKTKRNTNRRPQDVKFPGGGQIVGGVYLLPRRGPRLGVRAARKTSERSQPRGRRQPIPKDRRSPGKSWGKPGYPWPLYGNEGCGWAGWLLSPRGSRPTWGPTDPRHRSRNLGKVIDTITCGFADLMGYIPVVGAPVGGVARALAHGVRVLEDGINYATGNLPGCSFSIFLLALLSCLTVPASAVEVRNISSGYYXTNDCSNSSITWQLTNAVLHLPGCVPCENDNGTLRCWIQVTPNVAVKYRGALTHNLRTHVDMIVMAATVCSALYVGDVCGAVMIVSQAFIMSXERHNFTQECNCSIYQGHITGHRMAWDMMLGWSPTLTMILAYAARVPEXVLEVVFGGHWGVVFGLAYFSMQGAWAKVIAILLLVAGVDAGTYSSGATIGQGTRGLVXLFSAGPSQKISLINTNGSWHINRTXLNCNDSLQTGFIASLFYAKSFNSSGCPERLSSCRGLDDFRIGWGTLEYENNVTNDEDMRPYCWHYPPKPCGIVPARTVCGPVYCFTPSPVVVGTTDKQGVPTYSWGENETDVFLLNSTRPPQGAWFGCTWMNGTGFTKTCGAPPCRIRRDHTSTLDLLCPTDCFRKHPDTTYLKCGAGPWLTPKCLVDYPYRLWHYPCTXNFTIFKVRMYVGGVEHRFSAACNFTRGDRCRLEDRDRGQQSPLLHSTTEWAVLPCSFSDLPALSTGLLHLHQNIVDVQYLYGLSPAITKYIVKWEWVILLFLLLADARVCACLWMLIILGQAEA

SEQ ID NO: 40 - protein, 752 aa, Hepacivirus C
  VARIANT 431: note = X can be any naturally occurring amino acid
  VARIANT 466: note = X can be any naturally occurring amino acid
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIIAGAHWGILAGLAYYSMQGNWAKVAIIMVMFSGVDATTYTTGGSAARGARGLTSLFSVGAKQKLQLVNTNGSWHINSTALNCNXSINTGFIAGLFYYHKFNSTGCPQRLSSCKPITFFXQGWGPLTDANITGPSDDKPYCWHYAPRPCDVVPASSVCGPVYCFTPSPVVVGTTDAKGVPTYTWGENETDVFLLESLRPPSGRWFGCTWMNSTGFVKTCGAPPCNIYGGGGNPHNESDLFCPTDCFRKHPEATYSRCGAGPWLTPRCMVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQAEA

SEQ ID NO: 41 - protein, 752 aa, Hepacivirus C
MSTLPKPQRKTKRNTIRRPQDVKFPGGGQIVGGVYVLPRRGPRLGVRATRKTSERSQPRERRQPIPKARRSDGRSWAQPGYPWPLYGNEGCGWAGWLLSPRGSRPSWGPNDPRRRSRNLGKVIDTLTCGFADLMGYIPIVGAPVGGVARALAHGVRALEDGINFATGNLPGCSFSIFLLALLSCLIHPAASLEWRNTSGLYILTNDCPNSSIVYEADDVILHTPGCIPCVQDGNTSTCWTSVSPTVAVRYVGATTASIRSHVDLLVGAATLCSALYVGDMCGAVFLVGQAFTFRPRRHQTVQTCNCSLYPGHLTGHRMAWDMMMNWSPAVGMVVAHVLRMPQTVFDIIAGAHWGILAGLAYYSMQGNWAKVAIIMVMFSGVDAETHTTGGTAARNAFTLTGLFTQGARQKLELINTNGSWHINRTALNCNESLNTGFIAGLFYLHKFNSTGCPERLSSCKPITFFRQGWGSLTDANITGPSDDKPYCWHYAPRPCEVVPALNVCGPVYCFTPSPVVVGTTDRQGVPTYTWGENETDVFLLRSLRPPSGQWFGCTWMNSTGFVKTCGAPPCDIYGGGGNRCNESDLFCPTDCFRKHPEATYSRCGAGPWLTPRCLVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSGVVGWALRWEFVVLVFLLLADARVCVALWLMLMISQAEA

SEQ ID NO: 42 - protein, 752 aa, Hepacivirus C
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLVHPAASLEWRNTSGLYVLTNDCSNSSIVYEADDVILHTPGCVPCVQDDNTSTCWTPVTPTVAVRYVGATTASIRSHVDLLVGAATLCSALYVGDMCGAVFLVGQAFTFRPRRHQTVQTCNCSLYPGHVSGHRMAWDMMMNWSPAVGMVVAHILRLPQTLFDILAGAHWGILAGLAYYSMQGNWAKVAIVMIMFSGVDAETYVTGGSVAHSARGLTSLFSMGAKQKLQLVNTNGSWHINSTALNCNESINTGFIAGLFYYHKFNSTGCPQRLSSCKPIISFRQGWGPLTDANITGPSDDRPYCWHYAPRPCSVVPASSVCGPVYCFTPSPVVVGTTDIKGKPTYNWGENETDVFLLESLRPPSGRWFGCAWMNSTGFLKTCGAPPCNIYGGEGDPENETDLFCPTDCFRKHPEATYSRCGAGPWLTPRCMVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSDMVGWALKWEFVILVFLLLADARVCVALWLMLMVSQAEA

SEQ ID NO: 43 - protein, 752 aa, Hepacivirus C
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIMAGAHWGILAGLAYYSMQGNWAKVAIIMVMFSGVDAHTYTTGGTASRHTQAFAGLFDIGPQQKLQLVNTNGSWHINSTALNCNESINTGFIAGLFYYHKFNSTGCPQRLSSCKPITFFRQGWGPLTDANITGPSDDRPYCWHYAPRPCDIVPASSVCGPVYCFTPSPVVVGTTDARGVPTYTWGENEKDVFLLKSQRPPSGRWFGCSWMNSTGFLKTCGAPPCNIYGGEGNPHNESDLFCPTDCFRKHPETTYSRCGAGPWLTPRCMVDYPYRLWHYPCTVDFRLFKVRMFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQTEA

SEQ ID NO: 44 - protein, 752 aa, Hepacivirus C
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGQNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVSHVLRLPQTLFDIIAGAHWGILAGLAYYSMQGNWAKVAVIMVMFSGVDAETYITGGSAAHGVSTLTSLFSSGPQQKLQLVKTNGSWHINSTALNCNESINTGFIAGLFYYHKFNSTGCPQRLSSCKPITFFRQGWGSLTDANVTGASADKPYCWHYAPRPCDVVPALNVCGPVYCFTPSPVVVGTTDRKGVPTYNWGENESDVFLLESLRPPSGRWFGCAWMNSTGFLKTCGAPPCNIYGGGGNPNNESHLFCPTDCFRKHPDATYSRCGAGPWLTPRCMVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSGMVGWALKWEFVILIFLLLADARVCVALWLMLMISQAEA

SEQ ID NO: 45 - protein, 752 aa, Hepacivirus C
MSTLPKPQRKTKRNTIRRPQDVKFPGGGQIVGGVYVLPRRGPRLGVCATRKTSERSQPRRRRQPIPKARQSGGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQNDNISTCWTPVTPTVAVRYVGATTASIRSHVDLLVGAATMCSALYVGDMCGAVFLVGQAFTFRPRRHQTVQTCNCSLYPGHLSGHRMAWDMMMNWFPALGMAVAHVLRVPQTLFDIIAGAHWGILAGLAYYSMQGNWAKVAIIMVMFSGVDAVTYTTGGSAAHATRGLTSLFSVGAQQKLQLVNTNGSWHINSTALNCNESINTGFIAGLFYYHRFNSTGCPQRLSSCKPITFFKQGWGPLTDANISGPSDDKPYCWHYAPRPCKVVPASGVCGPVYCFTPSPVVVGTTDAKGVPTYTWGANDTDVFLLESLRPPGGRWFGCTWMNSTGFVKTCGASPCDIYGGGGNSGNESDLFCPTDCFRKHPEATYSRCGAGPWLTPRCMVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGVGSGMVGWALKWEFVILIFLLLADRRVCVALWLMLMITQAEA

SEQ ID NO: 46 - protein, 752 aa, Hepacivirus C
MSTLPKPQRKTKRNTVCRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNVSGLYILTNDCPNSSIVYEADDVILHTPGCIPCVQDGNTSTCWTALTPTVAVRYVGATTASIRSHVDLLVGAATMCSALYVGDMCGAVFLVGQAFTFRPRRHQTVQTCNCSLYPGHLTGHRMAWDMMMNWSPAVGMVVAHVLRLPQTLFDIIAGAHWGILAGLAYYSMQGNWAKVIIIMVMFSGVDATTHVTGGTAGLTAFRLTGLFTVGPQQKLQLVNTNGSWHINRTALNCNDSLNTGFIAGLFRFHKFNSTGCPEMLSSCKPITSFKQGWGPLTDANITIPSDDRPYCWHYPPRSCEVVPALSVCGPVYCFTPSPVVVGTTDAKGVPTYTWGENETDVFLLKSLRPPGGRWFGCTWMNSTGFVQTCGAPPCNIYGGGGDLKNESDLFCPTDCFRKHPEATYSRCGAGPWLTPRCMVDYPYRLWHYPCTVNFTLFKVRMFVGGFEHRFTAACNWTRGERCDIEDRDRSELHPLLHSTTELAILPCSFTPMPALSTGLIHLHQNIVDVQYLYGVGSGMVGWAVKWEFVILVFLLLADARVCVALWLMLMISQAEA

SEQ ID NO: 47 - protein, 752 aa, Hepacivirus C
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLQWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAAGMVVAHILRLPQTLFDIIAGAHWGILAGLAYYSMQGNWAKVAIIMVMFSGVDATTYTSGGSVAQQARGLADLFSVGAKQNLQLVNTNGSWHINSTALNCDDSINTGFIAGLFYYHKFNSTGCPQRLSDCKPITFFKQGWGPLTDANITGPSDDKPYCWHYAPRRCGVVPASSVCGPVYCFTPSPVVVGTTDAKGVPTYTWGANETDVFLLESLRPPSGRWFGCAWMNSTGFLKTCGAPPCNIYGGGGNPHNESDLFCPTDCFRKHPEATYSRCGAGPWLTPRCMVDYPYRLWHYPCTVNFTLFKMRTFVGGFEHRFTAACNWTRGERCDIEDRDRSEQHPLLHSTTELAILPCSFTPMPALSTGLIHLHQNIVDVQYLYGVGSGMVGWALKWEFVILVFLLLADARVCVALWLMLMISQAEA

SEQ ID NO: 48 - protein, 752 aa, Hepacivirus C
MSTLPKPQRKTKRNTIRRPQDVKFPGGGQIVGGVYVLPRRGPRLGVCATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLVHPAASLEWRNTSGLYVLTNDCPNSSIVYEADDVILHTPGCIPCVQDGNKSTCWTSVTPTVAVKYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIIAGAHWGILAGLAYYSMQGNWAKVAIIMVMFSGVDATTYTTGGNAARGASGIVSLFTPGAKQNLQLVNTNGSWHINRTALNCNDSINTGFIAGLIYYHKFNSTGCPQRLSSCKPITFFRQGWGSLTDANITGPSDDKPYCWHYPPRPCDTIRASSVCGPVYCFTPSPVVVGTTDAKGAPTYNWGANETDMFLLQSLRPPSGRWFGCTWMNSTGFTKTCGAPPCNIYGGGGNLNNESDLFCPTDCFRKHPEATYSRCGAGPWLTPRCLVDYPYRLWHYPCTVNFTLFRMRTFVGGFEHRFTAACNWTRGERCNIEDRDRSEQHPLLHSTTELAILPCSFTPMPALSTGLIHLHQNIVDVQYLYGIGSGVVGWALKWEFVILVFLLLADARVCVALWLMLMISQAEA

SEQ ID NO: 49 - protein, 752 aa, Hepacivirus C
MSTLPKPQRKTKRNTIRRPQDVKFPGGGVIYVGVYVLPRRGPRLGVRATRKTSERSQPRGRRKPIPKARRSEGRSWAQPGYPWPLYGNEGCGWAGWLLSPRGSRPNWAPNDPRRRSRNLGKVIDTLTCGFADLMGYIPLVGAPLGGAARALAHGVRALEDGINFATGNLPGCSFSIFLLALFSCLIHPAASLEWRNTSGLYVLTNDCSNSSIVYEADDVILHTPGCIPCVQDGNTSTCWTPVTPTVAVRYVGATTASIRSHVDLLVGAGTMCSALYVGDMCGPVFLVGQAFTFRPRRHRTVQTCNCSLYPGHLSGQRMAWDMMMNWSPAVGMVVAHILRLPQTLFDVVAGAHWGIIAGLAYYSMQGNWAKVAIIMVMFSGVDASTHVTAGQAARNAYGITSLFSVGAKQNLQLINTNGSWHINRTALNCNESINTGFIAGLFYYHKFNSTGCPQRLSSCKPITFFKQGWGPLTDANITGPSDDKPYCWHYAPRPCGIVPALNVCGPVYCFTPSPVVVGTTDAKGAPTYTWGANKTDVFLLESLRPPSGRWFGCTWMNSTGFVKTCGAPPCNIYGDGRDAQNESDLFCPTDCFRKHPEATYSRCGAGPWLTPRCLVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGIGSGMVGWALKWEFVILIFLLLADARVCVALWLILTISQAEA

SEQ ID NO: 50 - protein, 754 aa, Hepacivirus C
MSTLPKPKRQTKRNTLRRPKNVKFPAGGQIVGEVYVLPRRGPQLGVREVRKTSERSQPRGRRQPTPKARPREGRSWAQPGYPWPLYGNEGCGWAGWLLPPRGSRPSWGQNDPRRRSRNLGKVIDTLTCGFADLMGYIPLIGAPVGGVARALAHGVRALEDGVNYATGNLPGCSFSIFLLALFSCLTCPASSLEYRNASGLYLLTNDCSNRSIVYEADDVILHLPGCVPCVETDNNNTSCWTPISPTVAVKHPGVTTASIRNHVNMLVAPPTLCSALYVEDAFGAVSLVGQAFTFRPRQHKTVQTCNCSIYPGHVSGHRMAWDMMMNWSPAIGLVISHLMRLPQTFFDLVVGAHWGVMAGLAYFSMQGNWAKVVIVLIMFSGVDATTHTTGGSAAQATAGFTSFFTRGPSQNLQLVNSNGSWHINSTALNCNDSLNTGFIAGLFYYHKFNSSGCPERMSSCKPITYFNQGWGPLTDANINGPSEDRPYCWHYPPRPCNITKPLNVCGPVYCFTPSPVVVGTTDIKGLPTYRFGVNESDVFLLTSLRPPQGRWFGCVWMNSTGFVKTCGAPPCNIYGGMKDIEANQTHLKCPTDCFRKHHDATFTRCGSGPWLTPRCLVDYPYRLWHYPCTVNFSIFKVRMFVGGHEHRFSAACNWTRGERCDLEDRDRSEQQPLLHSTTDSLILPCSFTPMRRLSTGLIHLHQNIVDVQYLYGVGSAVVGWALKWEFVVLVFLLLADARVCVALWMMLLISQAEA

SEQ ID NO: 51 - protein, 751 aa, Hepacivirus C
MSTLPKPQRITKRNINRRPQDVKFPGGGQIVGGVYVLPRRGPKLGVRAVRKTSERSQPRSRRQPIPRARRTEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALLSCLLTPTAGLEYRNASGLYTVTNDCSNGSIVYEAGDVILHLPGCIPCVRLNNASKCWTPVSPTVAVSRPGAATASLRTHVDMMVGAATLCSALYVGDLCGALFLVGQGFSWRHRQHWTVQDCNCSIYPGHLTGHRMAWDMMMNWSPAMTLIVSQVLRLPQTMFDLVIGAHWGVMAGVAYYSMQGNWAKVFLVLCLFSGVDASTTITGGVAASGAFTITSLFSTGAKQPLHLVNTNGSWHINRTALNCNDSLNTGFIAGLLYYHKFNSSGCVERMSACSPLDRFAQGWGPLGPANISGPSSEKPYCWHYAPRPCDTVPAQSVCGPVYCFTPSPVVVGATDKRGAPTYTWGENESDVFLLESARPPTEPWFGCTWMNGSGYVKTCGAPPCHIYGGREGKSNNSLVCPTDCFRKHPDATYNRCGAGPWLTPRCLVDYPYRLWHYPCTVNYTIFKVRMFVGGLEHRFNAACNWTRGERCNLEDRDRSEMYPLLHSTTEQAILPCSFVPIPALSTGLIHLHQNIVDVQYLYGISSGLVGWAIKWEFVILIFLLLADARVCVVLWMMMLISQAEA

SEQ ID NO: 52 - protein, 36 aa, synthetic construct
  VARIANT 5: note = X is Glu or Ala
  VARIANT 8: note = X is Val, Thr, Tyr, Phe, or Leu
AIPLXEAXVTYFLIKGGRHLIFCHSKKKCDELAAKL

SEQ ID NO: 53 - protein, 56 aa, synthetic construct
  VARIANT 9: note = X is Val or Ile
  VARIANT 19: note = X is Arg or Ser
  VARIANT 45: note = X is Thr or Lys
LGRWTGTYXVIYDHLTPLXRSDWAAAGLRDLAVAVEPVVFSPMEXTKKVITWGADT

SEQ ID NO: 54 - protein, 60 aa, synthetic construct
  VARIANT 5: note = X is Glu or Ala
  VARIANT 8: note = X is Val, Thr, Phe, or Leu
  VARIANT 57: note = X is Ser or Gln
  VARIANT 58: note = X can be any naturally occurring amino acid
AIPLXEAXVTYFLIKGGRHLIFCHSKKKCDELAAKLRGMGLNAVAYYRGLDVSVIPTXSQ

SEQ ID NO: 55 - protein, 72 aa, synthetic construct
  VARIANT 44: note = X is Ser or Gln
KGGRHLIFCHSKKKCDELAAKLRGMGLNAVAYYRGLDVSVIPTXSQGDVVVVATDALMTGYTGDFDSVIDCN

SEQ ID NO: 56 - protein, 114 aa, synthetic construct
  VARIANT 5: note = X is Thr, Asn, Gln, His, or Ser
  VARIANT 24: note = X is Glu or Ala
  VARIANT 27: note = X is Val, Thr, Tyr, Phe, or Leu
  VARIANT 77: note = X is Ser or Gln
VALGXTNQHSEGEIPFYGKAIPLXEAXVTYFLIKGGRHLIFCHSKKKCDELAAKLRGMGLNAVAYYRGLDVSVIPTXSQGDVVVVATDALMTGYTGDFDSVIDCNVAVTQTVDF

SEQ ID NO: 57 - protein, 171 aa, synthetic construct
MSTNPKPQRKTKRNTNRRPQDVKFPGGGQIVGGVYLLPRRGPRLGVRATRKTSERSQPRGRRQPIPKARRSEGRSWAQPGYPWPLYGNEGCGWAGWLLSPRGSRPSWGPTDPRRRSRNLGKVIDTLTCGFADLMGYIPLVGAPLGGVARALAHGVRVLEDGVNYATGNLPG

SEQ ID NO: 58 - protein, 244 aa, synthetic construct
  VARIANT 94: note = X is Thr or Val
  VARIANT 144: note = X is Thr, Asn, Gln, His, or Ser
  VARIANT 163: note = X is Glu or Ala
  VARIANT 166: note = X is Val, Thr, Tyr, Phe, or Leu
  VARIANT 216: note = X is Ser or Gln
LHAPTGSGKSTKVPAAYAAQGYKVLVLNPSVAATLGFGAYMSKAHGIDPNIRTGVRTVTTGAPITYSTYGKFLADGGCSGGAYDIIICDECHSXTVDATTILGIGTVLDQAETAGARLVVLATATPPGSVTVPHPNIEEVALGXTNQHSEGEIPFYGKAIPLXEAXVTYFLIKGGRHLIFCHSKKKCDELAAKLRGMGLNAVAYYRGLDVSVIPTXSQGDVVVVATDALMTGYTGDFDSVIDCN

SEQ ID NO: 59 - protein, 631 aa, synthetic construct
  VARIANT 3: note = X can be any naturally occurring amino acid
  VARIANT 17: note = X can be any naturally occurring amino acid
  VARIANT 23: note = X can be any naturally occurring amino acid
  VARIANT 30: note = X can be any naturally occurring amino acid
  VARIANT 36: note = X can be any naturally occurring amino acid
  VARIANT 39: note = X can be any naturally occurring amino acid
  VARIANT 45: note = X can be any naturally occurring amino acid
  VARIANT 66: note = X can be any naturally occurring amino acid
  VARIANT 76: note = X can be any naturally occurring amino acid
  VARIANT 102: note = X can be any naturally occurring amino acid
  VARIANT 140: note = X can be any naturally occurring amino acid
  VARIANT 167: note = X can be any naturally occurring amino acid
  VARIANT 185: note = X can be any naturally occurring amino acid
  VARIANT 206: note = X can be any naturally occurring amino acid
  VARIANT 211: note = X can be any naturally occurring amino acid
  VARIANT 222: note = X can be any naturally occurring amino acid
  VARIANT 258: note = X can be any naturally occurring amino acid
  VARIANT 267: note = X can be any naturally occurring amino acid
  VARIANT 277: note = X can be any naturally occurring amino acid
  VARIANT 303: note = X can be any naturally occurring amino acid
  VARIANT 318: note = X can be any naturally occurring amino acid
  VARIANT 340: note = X can be any naturally occurring amino acid
  VARIANT 349: note = X can be any naturally occurring amino acid
  VARIANT 353: note = X can be any naturally occurring amino acid
  VARIANT 358: note = X can be any naturally occurring amino acid
  VARIANT 364: note = X can be any naturally occurring amino acid
  VARIANT 478: note = X can be any naturally occurring amino acid
  VARIANT 529: note = X can be any naturally occurring amino acid
  VARIANT 548: note = X can be any naturally occurring amino acid
  VARIANT 552: note = X can be any naturally occurring amino acid
  VARIANT 602: note = X can be any naturally occurring amino acid
QAXSALTRVPYFVRAHXGALLRXIAMVLCXMTLVRXKHXVLAGGXHRYVQMAILILKLGRWTGTYXVIYDHLTPLXRSDWAAAGLRDLAVAVEPVVFSPMEXTKKVITWGADTAACGDILHGLPVSARLGREILLGPADXGSYTSKGWRLLAPITAYAQQTRGLLGXCAIVTSLTGRDKNZQEVXEAGEVQVLSTATQSFLGTCIXNSGVXCLWTVYHGAGXSNKTLAGPKGPVTQMYTNVDQDLVGWPAPPGAKSLXTEPCTCGSXSVDLYLVTRXHNADVIPVRRRGDSRGSLLSPRPLSXYTLKGSSGGPVLCPXSRGHAVGIFRAAVCTRGVAKAXVIDFIPVEXSTLXEDTTXMTRSPXVSFSDNSTPPAVPQSYQVGYLHAPTGSGKSTKVPAAYAAQGYKVLVLNPSVAATLGFGAYMSKAHGIDPNIRTGVRTVTTGAPITYSTYGKFLADGGCSGGAYDIIICDECHSXTVQDATTILGIGTVLDQAETAGARLVVLATATPPGSVTVPHPNIEEVALGXTNQHSEGEIPFYGKAIPLXESAXVTYFLIKGGRHLIFCHSKKKCDELAAKLRGMGLNAVAYYRGLDVSVIPTXSQTGDVVVVATDALMTGYTGDFDSVIDCN

SEQ ID NO: 60 - protein, 865 aa, synthetic construct
  VARIANT 95: note = X can be any naturally occurring amino acid
  VARIANT 145: note = X can be any naturally occurring amino acid
  VARIANT 164: note = X can be any naturally occurring amino acid
  VARIANT 167: note = X can be any naturally occurring amino acid
  VARIANT 217: note = X can be any naturally occurring amino acid
  VARIANT 265: note = X can be any naturally occurring amino acid
  VARIANT 295: note = X can be any naturally occurring amino acid
  VARIANT 337: note = X can be any naturally occurring amino acid
  VARIANT 356: note = X can be any naturally occurring amino acid
  VARIANT 379: note = X can be any naturally occurring amino acid
  VARIANT 384: note = X can be any naturally occurring amino acid
  VARIANT 408: note = X can be any naturally occurring amino acid
  VARIANT 416: note = X can be any naturally occurring amino acid
  VARIANT 447: note = X can be any naturally occurring amino acid
  VARIANT 464: note = X can be any naturally occurring amino acid
  VARIANT 473: note = X can be any naturally occurring amino acid
  VARIANT 488: note = X can be any naturally occurring amino acid
  VARIANT 503: note = X can be any naturally occurring amino acid
  VARIANT 510: note = X can be any naturally occurring amino acid
  VARIANT 513: note = X can be any naturally occurring amino acid
  VARIANT 526: note = X can be any naturally occurring amino acid
  VARIANT 555: note = X can be any naturally occurring amino acid
  VARIANT 559: note = X can be any naturally occurring amino acid
  VARIANT 567: note = X can be any naturally occurring amino acid
  VARIANT 572: note = X can be any naturally occurring amino acid
  VARIANT 580: note = X can be any naturally occurring amino acid
  VARIANT 584: note = X can be any naturally occurring amino acid
  VARIANT 591: note = X can be any naturally occurring amino acid
  VARIANT 594: note = X can be any naturally occurring amino acid
  VARIANT 598: note = X can be any naturally occurring amino acid
  VARIANT 642: note = X can be any naturally occurring amino acid
  VARIANT 652: note = X can be any naturally occurring amino acid
  VARIANT 668: note = X can be any naturally occurring amino acid
  VARIANT 677: note = X can be any naturally occurring amino acid
  VARIANT 733: note = X can be any naturally occurring amino acid
  VARIANT 822: note = X can be any naturally occurring amino acid
  VARIANT 859: note = X can be any naturally occurring amino acid
YLHAPTGSGKSTKVPAAYAAQGYKVLVLNPSVAATLGFGAYMSKAHGIDPNIRTGVRTVTTGAPITYSTYGKFLADGGCSGGAYDIIICDECHSXTVDATTILGIGTVLDQAETAGARLVVLATATPPGSVTVPHPNIEEVALGXTNQHSEGEIPFYGKAIPLXEAXVTYFLIKGGRHLIFCHSKKKCDELAAKLRGMGLNAVAYYRGLDVSVIPTXSQGDVVVVATDALMTGYTGDFDSVIDCNVAVTQTVDFSLDPTFTIETXTQTVPQDAVSRSQRRGRTGRGRLGIYRYVXTSPGERPSGMFDSVVLCECYDAGCAWYELTPAETTVRLRAYXMLNTPGLPVCQDHLEFWEXSAVFTGLTHIDAHFLSQTKQAGXEDNFXPAYLVAYQATVCARAQAPPPSWDXQVMWKCLXITRLKPTLHGPTPLLYRLGAVQNEVTLTHPXIVTKYIMTCMSADLEVXVMTSTWVLXVAGGVLAALAAYCLXSATGCVVIVGRIHLXSNGKPAXIVXIAPDKEVLYZQEXEAFDEMEECASHAPYIEQGQRLIAEQFKXQSKXAILGLLQXTQATXRKQAZEQXVDIXAQPAVQXSTXSNWXQPKLEAFWAKHMWNFISGIQYLAGLSTLPGNPAVASLMAFTAAXVLTSPLTTSXQTTLILFNILGGWVAXASQLAPPAXAGATAFVVSGLAGAAVGSIGLGKVLVDILAGYGAGVSGALVAFKIMSGEKPSTEDXLVVNLLPAILSPGALVVGVICAAILRRHVGPGEGAVQWMNRLIAFASRGNHVSPTHYVPESDAAARVTQILLSSLTITSLLRRLHQWIXNTEDCPTPCSGSWLRDIWDWVCTVLTDFKTWLSSKLXMLPKLP

SEQ ID NO: 61 - protein, 2277 aa, synthetic construct
  VARIANT 16: note = X can be any naturally occurring amino acid
  VARIANT 34: note = X can be any naturally occurring amino acid
  VARIANT 55: note = X can be any naturally occurring amino acid
  VARIANT 60: note = X can be any naturally occurring amino acid
  VARIANT 71: note = X can be any naturally occurring amino acid
  VARIANT 107: note = X can be any naturally occurring amino acid
  VARIANT 116: note = X can be any naturally occurring amino acid
  VARIANT 126: note = X can be any naturally occurring amino acid
  VARIANT 152: note = X can be any naturally occurring amino acid
  VARIANT 167: note = X can be any naturally occurring amino acid
  VARIANT 189: note = X can be any naturally occurring amino acid
  VARIANT 198: note = X can be any naturally occurring amino acid
  VARIANT 202: note = X can be any naturally occurring amino acid
  VARIANT 207: note = X can be any naturally occurring amino acid
  VARIANT 213: note = X can be any naturally occurring amino acid
  VARIANT 327: note = X can be any naturally occurring amino acid
  VARIANT 377: note = X can be any naturally occurring amino acid
  VARIANT 396: note = X can be any naturally occurring amino acid
  VARIANT 399: note = X can be any naturally occurring amino acid
  VARIANT 449: note = X can be any naturally occurring amino acid
  VARIANT 497: note = X can be any naturally occurring amino acid
  VARIANT 527: note = X can be any naturally occurring amino acid
  VARIANT 569: note = X can be any naturally occurring amino acid
  VARIANT 588: note = X can be any naturally occurring amino acid
  VARIANT 611: note = X can be any naturally occurring amino acid
  VARIANT 616: note = X can be any naturally occurring amino acid
  VARIANT 640: note = X can be any naturally occurring amino acid
  VARIANT 648: note = X can be any naturally occurring amino acid
  VARIANT 679: note = X can be any naturally occurring amino acid
  VARIANT 696: note = X can be any naturally occurring amino acid
  VARIANT 705: note = X can be any naturally occurring amino acid
  VARIANT 720: note = X can be any naturally occurring amino acid
  VARIANT 735: note = X can be any naturally occurring amino acid
  VARIANT 742: note = X can be any naturally occurring amino acid
  VARIANT 745: note = X can be any naturally occurring amino acid
  VARIANT 758: note = X can be any naturally occurring amino acid
  VARIANT 787: note = X can be any naturally occurring amino acid
  VARIANT 791: note = X can be any naturally occurring amino acid
  VARIANT 799: note = X can be any naturally occurring amino acid
  VARIANT 804: note = X can be any naturally occurring amino acid
  VARIANT 812: note = X can be any naturally occurring amino acid
  VARIANT 816: note = X can be any naturally occurring amino acid
  VARIANT 823: note = X can be any naturally occurring amino acid
  VARIANT 826: note = X can be any naturally occurring amino acid
  VARIANT 830: note = X can be any naturally occurring amino acid
  VARIANT 874: note = X can be any naturally occurring amino acid
  VARIANT 884: note = X can be any naturally occurring amino acid
  VARIANT 900: note = X can be any naturally occurring amino acid
  VARIANT 909: note = X can be any naturally occurring amino acid
  VARIANT 965: note = X can be any naturally occurring amino acid
  VARIANT 1054: note = X can be any naturally occurring amino acid
  VARIANT 1091: note = X can be any naturally occurring amino acid
  VARIANT 1140: note = X can be any naturally occurring amino acid
  VARIANT 1148: note = X can be any naturally occurring amino acid
  VARIANT 1155: note = X can be any naturally occurring amino acid
  VARIANT 1159: note = X can be any naturally occurring amino acid
  VARIANT 1168: note = X can be any naturally occurring amino acid
  VARIANT 1185: note = X can be any naturally occurring amino acid
  VARIANT 1188: note = X can be any naturally occurring amino acid
  VARIANT 1198: note = X can be any naturally occurring amino acid
  VARIANT 1217: note = X can be any naturally occurring amino acid
  VARIANT 1250: note = X can be any naturally occurring amino acid
  VARIANT 1264: note = X can be any naturally occurring amino acid
  VARIANT 1342: note = X can be any naturally occurring amino acid
  VARIANT 1371: note = X can be any naturally occurring amino acid
  VARIANT 1399: note = X can be any naturally occurring amino acid
  VARIANT 1427: note = X can be any naturally occurring amino acid
  VARIANT 1436: note = X can be any naturally occurring amino acid
  VARIANT 1450: note = X can be any naturally occurring amino acid
  VARIANT 1475: note = X can be any naturally occurring amino acid
  VARIANT 1489: note = X can be any naturally occurring amino acid
  VARIANT 1496: note = X can be any naturally occurring amino acid
  VARIANT 1501: note = X can be any naturally occurring amino acid
  VARIANT 1504: note = X can be any naturally occurring amino acid
  VARIANT 1508: note = X can be any naturally occurring amino acid
  VARIANT 1511: note = X can be any naturally occurring amino acid
  VARIANT 1524: note = X can be any naturally occurring amino acid
  VARIANT 1532: note = X can be any naturally occurring amino acid
  VARIANT 1537: note = X can be any naturally occurring amino acid
  VARIANT 1549: note = X can be any naturally occurring amino acid
  VARIANT 1577: note = X can be any naturally occurring amino acid
  VARIANT 1580: note = X can be any naturally occurring amino acid
  VARIANT 1590: note = X can be any naturally occurring amino acid
  VARIANT 1593: note = X can be any naturally occurring amino acid
  VARIANT 1598: note = X can be any naturally occurring amino acid
  VARIANT 1620: note = X can be any naturally occurring amino acid
  VARIANT 1643: note = X can be any naturally occurring amino acid
  VARIANT 1671: note = X can be any naturally occurring amino acid
  VARIANT 1677: note = X can be any naturally occurring amino acid
  VARIANT 1726: note = X can be any naturally occurring amino acid
  VARIANT 1745: note = X can be any naturally occurring amino acid
  VARIANT 1748: note = X can be any naturally occurring amino acid
  VARIANT 1772: note = X can be any naturally occurring amino acid
  VARIANT 1813: note = X can be any naturally occurring amino acid
  VARIANT 1824: note = X can be any naturally occurring amino acid
  VARIANT 1841: note = X can be any naturally occurring amino acid
  VARIANT 1861: note = X can be any naturally occurring amino acid
  VARIANT 1876: note = X can be any naturally occurring amino acid
  VARIANT 1923: note = X can be any naturally occurring amino acid
  VARIANT 1946: note = X can be any naturally occurring amino acid
  VARIANT 1963: note = X can be any naturally occurring amino acid
  VARIANT 1968: note = X can be any naturally occurring amino acid
  VARIANT 1972: note = X can be any naturally occurring amino acid
  VARIANT 1980: note = X can be any naturally occurring amino acid
  VARIANT 1984: note = X can be any naturally occurring amino acid
  VARIANT 2025: note = X can be any naturally occurring amino acid
  VARIANT 2029: note = X can be any naturally occurring amino acid
  VARIANT 2039: note = X can be any naturally occurring amino acid
  VARIANT 2098: note = X can be any naturally occurring amino acid
  VARIANT 2102: note = X can be any naturally occurring amino acid
  VARIANT 2106: note = X can be any naturally occurring amino acid
  VARIANT 2117: note = X can be any naturally occurring amino acid
  VARIANT 2148: note = X can be any naturally occurring amino acid
  VARIANT 2157: note = X can be any naturally occurring amino acid
  VARIANT 2160: note = X can be any naturally occurring amino acid
  VARIANT 2168: note = X can be any naturally occurring amino acid
  VARIANT 2186: note = X can be any naturally occurring amino acid
  VARIANT 2201: note = X can be any naturally occurring amino acid
  VARIANT 2223: note = X can be any naturally occurring amino acid
  VARIANT 2237: note = X can be any naturally occurring amino acid
  VARIANT 2255: note = X can be any naturally occurring amino acid
APITAYAQQTRGLLGXCAIVTSLTGRDKNZQEVXEAGEVQVLSTATQSFLGTCIXNSGVXCLWTVYHGAGXSNKTLAGPKGPVTQMYTNVDQDLVGWPAPPGAKSLXTEPCTCGSXSVDLYLVTRXHNADVIPVRRRGDSRGSLLSPRPLSXYTLKGSSGGPVLCPXSRGHAVGIFRAAVCTRGVAKAXVIDFIPVEXSTLXEDTTXMTRSPXVSFSDNSTPPAVPQSYQVGYLHAPTGSGKSTKVPAAYAAQGYKVLVLNPSVAATLGFGAYMSKAHGIDPNIRTGVRTVTTGAPITYSTYGKFLADGGCSGGAYDIIICDECHSXTVDATTILGIGTVLDQAETAGARLVVLATATPPGSVTVPHPNIEEVALGXTNQHSEGEIPFYGKAIPLXEAXVTYFLIKGGRHLIFCHSKKKCDELAAKLRGMGLNAVAYYRGLDVSVIPTXSQGDVVVVATDALMTGYTGDFDSVIDCNVAVTQTVDFSLDPTFTIETXTQTVPQDAVSRSQRRGRTGRGRLGIYRYVXTSPGERPSGMFDSVVLCECYDAGCAWYELTPAETTVRLRAYXMLNTPGLPVCQDHLEFWEXSAVFTGLTHIDAHFLSQTKQAGXEDNFXPAYLVAYQATVCARAQAPPPSWDXQVMWKCLXITRLKPTLHGPTPLLYRLGAVQNEVTLTHPXIVTKYIMTCMSADLEVXVMTSTWVLXVAGGVLAALAAYCLXSATGCVVIVGRIHLXSNGKPAXIVXIAPDKEVLYZQEXEAFDEMEECASHAPYIEQGQRLIAEQFKXQSKXAILGLLQXTQATXRKQAZEQXVDIXAQPAVQXSTXSNWXQPKLEAFWAKHMWNFISGIQYLAGLSTLPGNPAVASLMAFTAAXVLTSPLTTSXQTTLILFNILGGWVAXASQLAPPAXAGATAFVVSGLAGAAVGSIGLGKVLVDILAGYGAGVSGALVAFKIMSGEKPSTEDXLVVNLLPAILSPGALVVGVICAAILRRHVGPGEGAVQWMNRLIAFASRGNHVSPTHYVPESDAAARVTQILLSSLTITSLLRRLHQWIXNTEDCPTPCSGSWLRDIWDWVCTVLTDFKTWLSSKLXMLPKLPGLPFISCQKGYKGVWRGDGIMTTRCPCGANITGHVKNGSMRIXVTGPKTCXRSMLANXMTWXHQGTFPINXACYTTGPCTPKPAPNYXTKXRTALWRVAAXESEYVEVTQVGDFHYVTGXMLTTDNLKCPCQVPAPEFFTEVDGVRLHRFAPXPTCKPLLRDEVTFXRQCTMVGLNSYVVGSQLPCEPEPDVAVLTSMLTDPSHITAEAAARRLARGSPPSEASSSASQLSAPSLKATCTTHHDXSAPDADLVDANLLWRQEMGGNITRVESEXNSKVVILDSFDPLRAEEDDREPSVPAEXIYLRKSRKFPPALPIWARPDYNPPLLEXTSWKRPDYXEVPPTVHGCALPPXPTKAPPVPPPRRKRTVVLTESTVSXTSDGAALAELATKXSTFGSSXEPQKXPDXSDSXSGXGVLHSSTGADTAXTPDGSSSXEGVTXPRQSQPSDEPAXDGLAESDAESYSSMPPLEGEPGDPDLSXDGXECGSWSTVSXEDXASEEXEDSVVCCSMSYSWTGALITPCXASAEEEKLPINPLSNSLLRHHNXLKVYSTTSRSASQRQKKVTFDRLQVLDXDAHYDXDSVLKEVKAAASKVKARLLSVEEACALTPPHSAKSKFGYGAKDVRSLSXRSKAVNHIRSVWKDLLEDXSTXVEQHTTPIDTTIMAKNEVFCVDPXETKGGRKPARLIVYPDLGVRVCEKMALYDVVQKLPQAVMGXSPSYGFQYSPXGAQRVEFLLKAWKSKKXTDPMGFSYDTRCFDSTVTEXSRDIRTEESIYQCCXDSLPPEARTAIHSLTERLYVGGPMTNSKGQNCGYRRCRASGVLTTSXCMGNTILTCYIKALAACKAAGLXQVDPTMLVCGDDLVVIXCSESXAQGXVTZQEEDXAEAXSNLRAFTEAMTRYSAPPGDPPQPEYDLELITSCSSNVSVAXHLDXGAPSDRGKRXVRYYLTRDPTTPLARAAWETARHTPVNSWLGNIIMYAPTIWVRMVLMTHFFSILLAQDXQTLXEDQXANLDFEMYGAXCVYSVEPLDLPAIIERLHGLSAFSLHSYSPXGHELNRVAXASXCTLRKLGXVAPPLRAWRHRARAVRAXKSLISQGGRAAICGXKRYLFNWAVRTKLKLTPLPAAXGSQLDLSGWFTVGXYAGGGDIYHSVSRARPRXWLLLLCLLLLSVGVGIFLLPAR

SEQ ID NO: 62 - DNA, 1740 bp, synthetic construct
  source 1..1740: mol_type = other DNA
gccaccatggacgcaatgaaacggggtctgtgctgcgtactcttgctttgtggagctgtgttcgtgtcaccttcttaccaggtgaggaattcatccggactgtaccatgtgacaaatgattgcccgaacagtagcatcgtctatgaggcggcggatgcgatactccatacgccagggtgtgtaccatgtgtaagggaaggaaatgctagtcgatgctgggttgcagtcacacctacagtggccacacgagacggcaaattgccaactactcagttgcggcgccacatagatctcttggtcggtagtgctacactgtgctctgccctgtacgtcggagacctctgtggatcagtttttctcgtggggcaactttttacattttcacctcgccggcattggaccacacaggactgtaactgttccatctatccaggccatattaccggtcatagaatggcatgggacatgatgatgaattggtctccgactgctgcattggtcgtagcccaactgctgaggatcccccaagccattatggatatgatagcgggggcacactggggggtgctcgccgggatcgcttacttctctatggtagggaactgggccaaggttctggtggtcctgctgctttttgcaggggtggatgccgagacgcatgtgacgggtgggaatgcaggtcgcaccactgctggtctcgtggggctgttgactcccggagcgaagcagaatattcagcttattaacacaaacgggtcctggcacattaattctacagcgctgaactgcaatgaaagtctcaatacgggttggctcgcggggttgttttatcaacacaagtttaactcctcaggatgtccagagcgcctcgcttcctgcagaagattgacggatttcgctcagggatgggggcccatctcatatgcaaacggttcaggccttgacgaaagaccatactgttggcactatccacctcgaccgtgtggaatcgtgccagcgaaatctgtctgtgggccagtctactgctttaccccatcacccgttgtagtgggaaccactgacagaagtggggcccccacctacagttggggggctaacgataccgacgtattcgtcctgaataatacccggccaccgcttggtaactggttcggttgtacctggatgaacagtacagggttcaccaaggtttgcggtgctccaccgtgcgtgataggtggcgttgggaataacacgctgctctgcccaaccgactgtttccgaaagcacccggaggcgacgtattcaaggtgcgggtcagggccctggatcactccccgctgtatggtagattacccgtacaggctgtggcactatccttgtactatcaattacaccatcttcaaagtacggatgtatgtggggggcgtcgagcaccgacttgaagctgcatgtaattggactcggggggagcgatgtgacctggaggatagggaccgcagcgaactctcacctctcctgctttctaccactcagtggcaagtcctgccctgtagtttcacgacattgccggcattgtcaactggtttgatccacctgcatcaaaacatcgttgacgtgcaatacctgtatggcgtaggcagcagtatcgcgtcctgggcgataaaatgggagtacgttgtactcctgtttttgttgctggccgatgcgcgagtttgcagttgcttgtggatgatgcttctcatttcacaagcggaatga

SEQ ID NO: 63 - protein, 23 aa, synthetic construct
MDAMKRGLCCVLLLCGAVFVSPS

SEQ ID NO: 64 - DNA, 1755 bp, synthetic construct
  source 1..1755: mol_type = other DNA
gccaccatggacgcaatgaaacggggtctgtgctgcgtactcttgctttgtggagctgtgttcgtgtcaccttctgcggaggtcaaaaacatctctacgggttatatggttactaacgactgcacgaatgattctataacgtggcaacttcaagcagccgtattgcatgttcctggatgcgtaccttgcgagaaggttgggaatgcttctcaatgctggattcccgttagccccaacgtagcggtgcaaagacctggggcccttacccagggcctcagaacccacatcgatatggttgtaatgagcgcaacactttgttccgcactttatgttggagacttgtgcggtggtgtaatgttggctgcgcaaatgttcattgtaagccctcagcatcattggttcgttcaagactgcaactgttccatttaccccgggactatcactggtcacaggatggcgtgggacatgatgatgaactggagccctaccgcgaccatgatactggcgtacgctatgcgcgtgcccgaagttattattgatattatctccggcgcacattggggagttatgtttggcctggcatatttctctatgcaaggtgcgtgggcaaaggtagttgtcatacttttgttggcagcaggagtagatgcgcgaacccacaccgtcggagggtcagccgcacaaactaccggaaggttgacgagtttgtttgatatgggcccacgacaaaagattcaattggttaatacgaacgggtcctggcatattaacagaactgcacttaactgcaatgactcccttcatactggattcatcgcaagtcttttctatacacacagttttaacagctcaggctgtcccgagaggatgagcgcatgccgctccatcgaggcctttagggtaggttggggagcgctgcagtacgaagacaatgtgaccaatcccgaagacatgcggccgtactgttggcactacccaccacgacaatgtggtgtagtttcagcgaaaacggtttgtggcccagtttactgctttaccccttcaccagtcgtggttgggaccaccgacaggctgggagcccctacctatacctggggtgaaaacgaaaccgatgtctttctcctcaactctacaaggccccccctgggatcctggttcggctgtacttggatgaattcctcagggtacacgaagacgtgtggagccccgccttgccgcacgagggcggattttaatgcgtccaccgaccttttgtgcccgactgactgttttcgcaagcaccccgatacgacttacctcaagtgtggttccggtccgtggctcacgccgcgatgcttgatagactatccttaccggctttggcactacccttgtactgtgaattatactatattcaagattcgcatgtacgtgggcggggttgaacataggttgactgcggcttgcaatttcactagaggagatagatgcaacctcgaggatcgggataggagtcaactgtcccctctcctccacagcacgacagagtgggccatactgccatgttcttactctgaccttccggcgttgtccacggggcttcttcacctgcatcagaatattgtagatgttcagttcatgtacggcctgtctcctgcgctcacaaaatatatcgttcgatgggagtgggttatcctcctcttccttctgctcgcggatgcgcgggtatgtgcgtgcctctggatgcttatactgcttggccaggcagaggcctga

SEQ ID NO: 65 - DNA, 1761 bp, synthetic construct
  source 1..1761: mol_type = other DNA
gccaccatggacgcaatgaaacggggtctgtgctgcgtactcttgctttgtggagctgtgttcgtgtcaccttctctggaatggcgaaacacatcaggcctgtacgtattgaccaacgattgcagcaactcctctatagtctatgaagcggacgatgtcatattgcacaccccgggatgtgtaccttgtgtacaggatgacaatacgtcaacttgttggacgccggttactccgactgtagcagtccgctacgtcggagctacaacagcgagtatacgctcacatgtagatttgctggtaggtgcagcaaccctctgtagcgctctgtatgttggcgacatgtgcggggcagtttttcttgtggggcaggcctttacttttagacccagaagacaccaaactgtgcaaacttgtaattgtagcctttaccctggccatgtcagcggtcacagaatggcctgggatatgatgatgaactggtcacccgctgtgggtatggtcgttgctcacatcctgcgacttccacagactcttttcgatatccttgctggtgcgcactgggggatacttgctggtctggcctactattcaatgcaagggaactgggcaaaagtggccatcgtcatgattatgttttctggagtcgatgcggagacatatgtcacgggggggtctgtggcgcattcagcacgcggactgacatcattgttctctatgggggccaaacaaaaactgcaactcgttaataccaacggctcatggcacattaacagcaccgccctcaactgtaatgaatccattaacacggggtttattgcagggttgttttattaccacaaatttaacagtacagggtgcccacagaggctgtctagttgccgccctataatcagctttagacagggatggggccctttgactgacgccaatattacgggtccttccgacgatagaccctactgttggcattatgccccgcgaccgtgcggtgtcgtacctgcttcatctgtctgtggcccggtttattgctttactccaagccccgtcgtggtgggcacgacagatatcaaaggacgacctacgtataattggggcgaaaatgaaactgatgtatttcttctggaaagtcttcgaccgccaagtggtcggtggtttggctgtgcctggatgaatagtactgggttccttaaaacgtgtggcgcgcctccttgtaacatttacggtggggaaggcgatcctgagaatgaaacagacttgttctgtcctacggactgttttagaaagcacccggaagccacctattcaaggtgcggtgcgggaccgtggttgaccccaaggtgcatggtggactatccttaccggctgtggcattacccgtgcaccgtaaattttactcttttcaaagttaggatgtttgttggaggctttgaacatcggtttactgcagcttgtaactggacgcgcggggagcgctgtaacattgaggatagggataggtctgaacaacatccgttgctgcattcaacaacagaacttgcgatccttccatgtagtttcacacccatgcccgctttgtcaacaggtctcatccaccttcaccaaaatatagttgacgtgcagtatctctacggcgtgggtagtgatatggttggatgggcactgaaatgggaatttgtcatattggtattccttctgctggcagacgctcgagtatgcgtagccctttggctcatgctgatgatatctcaggcggaggcctga

SEQ ID NO: 66 - DNA, 492 bp, synthetic construct
  source 1..492: mol_type = other DNA
gccaccatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtga

SEQ ID NO: 67 - DNA, 150 bp, synthetic construct
  source 1..150: mol_type = other DNA
ggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctacccc

SEQ ID NO: 68 - DNA, 105 bp, synthetic construct
  source 1..105: mol_type = other DNA
aaatctaccaaggtgcctgccgcttacgccgcccagggctacaaggtcctggtgctgaaccccagcgtggccgctacactgggattcggcgcctacatgagcaag

SEQ ID NO: 69 - DNA, 81 bp, synthetic construct
  source 1..81: mol_type = other DNA
ctggaacagatcaagggcggcagacacctgatcttctgtcacagcaagaaaaagtgcgacgagctggccgccaagctgcgg

SEQ ID NO: 70 - DNA, 144 bp, synthetic construct
  source 1..144: mol_type = other DNA
atcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctg

SEQ ID NO: 71 - RNA, 1734 nt, synthetic construct
  source 1..1734: mol_type = other RNA
atggacgcaatgaaacggggtctgtgctgcgtactcttgctttgtggagctgtgttcgtgtcaccttcttaccaggtgaggaattcatccggactgtaccatgtgacaaatgattgcccgaacagtagcatcgtctatgaggcggcggatgcgatactccatacgccagggtgtgtaccatgtgtaagggaaggaaatgctagtcgatgctgggttgcagtcacacctacagtggccacacgagacggcaaattgccaactactcagttgcggcgccacatagatctcttggtcggtagtgctacactgtgctctgccctgtacgtcggagacctctgtggatcagtttttctcgtggggcaactttttacattttcacctcgccggcattggaccacacaggactgtaactgttccatctatccaggccatattaccggtcatagaatggcatgggacatgatgatgaattggtctccgactgctgcattggtcgtagcccaactgctgaggatcccccaagccattatggatatgatagcgggggcacactggggggtgctcgccgggatcgcttacttctctatggtagggaactgggccaaggttctggtggtcctgctgctttttgcaggggtggatgccgagacgcatgtgacgggtgggaatgcaggtcgcaccactgctggtctcgtggggctgttgactcccggagcgaagcagaatattcagcttattaacacaaacgggtcctggcacattaattctacagcgctgaactgcaatgaaagtctcaatacgggttggctcgcggggttgttttatcaacacaagtttaactcctcaggatgtccagagcgcctcgcttcctgcagaagattgacggatttcgctcagggatgggggcccatctcatatgcaaacggttcaggccttgacgaaagaccatactgttggcactatccacctcgaccgtgtggaatcgtgccagcgaaatctgtctgtgggccagtctactgctttaccccatcacccgttgtagtgggaaccactgacagaagtggggcccccacctacagttggggggctaacgataccgacgtattcgtcctgaataatacccggccaccgcttggtaactggttcggttgtacctggatgaacagtacagggttcaccaaggtttgcggtgctccaccgtgcgtgataggtggcgttgggaataacacgctgctctgcccaaccgactgtttccgaaagcacccggaggcgacgtattcaaggtgcgggtcagggccctggatcactccccgctgtatggtagattacccgtacaggctgtggcactatccttgtactatcaattacaccatcttcaaagtacggatgtatgtggggggcgtcgagcaccgacttgaagctgcatgtaattggactcggggggagcgatgtgacctggaggatagggaccgcagcgaactctcacctctcctgctttctaccactcagtggcaagtcctgccctgtagtttcacgacattgccggcattgtcaactggtttgatccacctgcatcaaaacatcgttgacgtgcaatacctgtatggcgtaggcagcagtatcgcgtcctgggcgataaaatgggagtacgttgtactcctgtttttgttgctggccgatgcgcgagtttgcagttgcttgtggatgatgcttctcatttcacaagcggaatga

SEQ ID NO: 72 - RNA, 1749 nt, synthetic construct
  source 1..1749: mol_type = other RNA
atggacgcaatgaaacggggtctgtgctgcgtactcttgctttgtggagctgtgttcgtgtcaccttctgcggaggtcaaaaacatctctacgggttatatggttactaacgactgcacgaatgattctataacgtggcaacttcaagcagccgtattgcatgttcctggatgcgtaccttgcgagaaggttgggaatgcttctcaatgctggattcccgttagccccaacgtagcggtgcaaagacctggggcccttacccagggcctcagaacccacatcgatatggttgtaatgagcgcaacactttgttccgcactttatgttggagacttgtgcggtggtgtaatgttggctgcgcaaatgttcattgtaagccctcagcatcattggttcgttcaagactgcaactgttccatttaccccgggactatcactggtcacaggatggcgtgggacatgatgatgaactggagccctaccgcgaccatgatactggcgtacgctatgcgcgtgcccgaagttattattgatattatctccggcgcacattggggagttatgtttggcctggcatatttctctatgcaaggtgcgtgggcaaaggtagttgtcatacttttgttggcagcaggagtagatgcgcgaacccacaccgtcggagggtcagccgcacaaactaccggaaggttgacgagtttgtttgatatgggcccacgacaaaagattcaattggttaatacgaacgggtcctggcatattaacagaactgcacttaactgcaatgactcccttcatactggattcatcgcaagtcttttctatacacacagttttaacagctcaggctgtcccgagaggatgagcgcatgccgctccatcgaggcctttagggtaggttggggagcgctgcagtacgaagacaatgtgaccaatcccgaagacatgcggccgtactgttggcactacccaccacgacaatgtggtgtagtttcagcgaaaacggtttgtggcccagtttactgctttaccccttcaccagtcgtggttgggaccaccgacaggctgggagcccctacctatacctggggtgaaaacgaaaccgatgtctttctcctcaactctacaaggccccccctgggatcctggttcggctgtacttggatgaattcctcagggtacacgaagacgtgtggagccccgccttgccgcacgagggcggattttaatgcgtccaccgaccttttgtgcccgactgactgttttcgcaagcaccccgatacgacttacctcaagtgtggttccggtccgtggctcacgccgcgatgcttgatagactatccttaccggctttggcactacccttgtactgtgaattatactatattcaagattcgcatgtacgtgggcggggttgaacataggttgactgcggcttgcaatttcactagaggagatagatgcaacctcgaggatcgggataggagtcaactgtcccctctcctccacagcacgacagagtgggccatactgccatgttcttactctgaccttccggcgttgtccacggggcttcttcacctgcatcagaatattgtagatgttcagttcatgtacggcctgtctcctgcgctcacaaaatatatcgttcgatgggagtgggttatcctcctcttccttctgctcgcggatgcgcgggtatgtgcgtgcctctggatgcttatactgcttggccaggcagaggcctga

SEQ ID NO: 73 - RNA, 1755 nt, synthetic construct
  source 1..1755: mol_type = other RNA
atggacgcaatgaaacggggtctgtgctgcgtactcttgctttgtggagctgtgttcgtgtcaccttctctggaatggcgaaacacatcaggcctgtacgtattgaccaacgattgcagcaactcctctatagtctatgaagcggacgatgtcatattgcacaccccgggatgtgtaccttgtgtacaggatgacaatacgtcaacttgttggacgccggttactccgactgtagcagtccgctacgtcggagctacaacagcgagtatacgctcacatgtagatttgctggtaggtgcagcaaccctctgtagcgctctgtatgttggcgacatgtgcggggcagtttttcttgtggggcaggcctttacttttagacccagaagacaccaaactgtgcaaacttgtaattgtagcctttaccctggccatgtcagcggtcacagaatggcctgggatatgatgatgaactggtcacccgctgtgggtatggtcgttgctcacatcctgcgacttccacagactcttttcgatatccttgctggtgcgcactgggggatacttgctggtctggcctactattcaatgcaagggaactgggcaaaagtggccatcgtcatgattatgttttctggagtcgatgcggagacatatgtcacgggggggtctgtggcgcattcagcacgcggactgacatcattgttctctatgggggccaaacaaaaactgcaactcgttaataccaacggctcatggcacattaacagcaccgccctcaactgtaatgaatccattaacacggggtttattgcagggttgttttattaccacaaatttaacagtacagggtgcccacagaggctgtctagttgccgccctataatcagctttagacagggatggggccctttgactgacgccaatattacgggtccttccgacgatagaccctactgttggcattatgccccgcgaccgtgcggtgtcgtacctgcttcatctgtctgtggcccggtttattgctttactccaagccccgtcgtggtgggcacgacagatatcaaaggacgacctacgtataattggggcgaaaatgaaactgatgtatttcttctggaaagtcttcgaccgccaagtggtcggtggtttggctgtgcctggatgaatagtactgggttccttaaaacgtgtggcgcgcctccttgtaacatttacggtggggaaggcgatcctgagaatgaaacagacttgttctgtcctacggactgttttagaaagcacccggaagccacctattcaaggtgcggtgcgggaccgtggttgaccccaaggtgcatggtggactatccttaccggctgtggcattacccgtgcaccgtaaattttactcttttcaaagttaggatgtttgttggaggctttgaacatcggtttactgcagcttgtaactggacgcgcggggagcgctgtaacattgaggatagggataggtctgaacaacatccgttgctgcattcaacaacagaacttgcgatccttccatgtagtttcacacccatgcccgctttgtcaacaggtctcatccaccttcaccaaaatatagttgacgtgcagtatctctacggcgtgggtagtgatatggttggatgggcactgaaatgggaatttgtcatattggtattccttctgctggcagacgctcgagtatgcgtagccctttggctcatgctgatgatatctcaggcggaggcctga

SEQ ID NO: 74 - RNA, 486 nt, synthetic construct
  source 1..486: mol_type = other RNA
atgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtga

SEQ ID NO: 75 - RNA, 150 nt, synthetic construct
  source 1..150: mol_type = other RNA
ggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctacccc

SEQ ID NO: 76 - RNA, 105 nt, synthetic construct
  source 1..105: mol_type = other RNA
aaatctaccaaggtgcctgccgcttacgccgcccagggctacaaggtcctggtgctgaaccccagcgtggccgctacactgggattcggcgcctacatgagcaag

SEQ ID NO: 77 - RNA, 81 nt, synthetic construct
  source 1..81: mol_type = other RNA
ctggaacagatcaagggcggcagacacctgatcttctgtcacagcaagaaaaagtgcgacgagctggccgccaagctgcgg

SEQ ID NO: 78 - RNA, 144 nt, synthetic construct
  source 1..144: mol_type = other RNA
atcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctg

SEQ ID NO: 79 - DNA, 1743 bp, synthetic construct
  source 1..1743: mol_type = other DNA
gccaccatggacgcaatgaaacggggtctgtgctgcgtactcttgctttgtggagctgtgttcgtgtcaccttcttacgaggtgagaaatgtatccggagtctaccatgtaacgaacgactgctctaacgccagtatcgtatacgaggcggccgacatgattatgcacaccccgggctgcgtcccttgtgttcgggaaaataatagtagcagatgctgggtcgcgttgacgccaaccctcgcagcgcgaaacgcatcagttccgacgacgactatccgcagacatgttgatctcttggtgggagcggctgcgttgtgtagtgcaatgtatgtcggagatctttgtgggtctgtcttccttgtcgcccaactgttcacgttttccccaagacgccatgaaacggtacaagattgtaattgttcaatatacccgggacacgtcacgggacacaggatggcctgggatatgatgatgaattggagtccaaccgcagctttggtcgtaagtcaacttttgcgaatcccccaggccgtggttgacatggtagcgggcgcgcactggggagtgcttgcgggactcgcatattacagtatggttgggaattgggcgaaggtcttgattgtgatgttgctgtttgctggagtcgacggtggtacgtacgtgaccgggggcaccatggccaagaacaccctcgggattacctctcttttttccccgggctcatcccaaaaaatacaacttgtgaacacgaacggctcttggcatattaatcgaacagcattgaactgtaatgactccctcaacacggggttcttggcggcattgttttatgtgcacaagtttaacagctccggctgccccgaacggatggcgagttgttccccaatcgacgccttcgctcaaggatgggggccgattacatataatgaaagtcatagtagcgatcagaggccgtactgctggcactacgcgccaagaccatgcggcatcgtccctgcagcacaagtctgcgggcctgtttattgcttcactccgagccccgtagttgtgggcactactgacaggttcggagtgccgacttatagttggggggagaatgagaccgacgtcctcttgctcaataacaccaggcctccgcagggtaattggttcggctgtacttggatgaatagcacggggtttaccaaaacatgcggaggacccccctgtaacattggaggaataggtaacaaaaccctgacatgtccgacggattgcttccgcaaacatccagaggcaacctacactaaatgtggtagtgggccttggctgacgccacgctgtcttgttcactatccgtatagactgtggcattatccgtgcactgtcaactttacgatcttcaaagtgaggatgtatgtcgggggcgttgaacacagacttgaagcggcctgcaattggactcgaggcgaacgctgtaatcttgaagaccgggaccgatctgagttgtcacctcttcttttgagcaccactgagtggcaagtgctgccatgcagcttcacaacactcccagcactctcaaccgggcttatccacttgcaccaaaacgtggtcgatgtccaatatttgtatgggatcggcagcgcagttgtgagtttcgccattaagtgggaatatgtactccttctgttcctgctcttggcagacgcgcgagtctgcgcatgcttgtggatgatgctcctcattgctcaagcagaggcatga

SEQ ID NO: 80 - DNA, 2648 bp, synthetic construct
  source 1..2648: mol_type = other DNA
gccaccatggacgcaatgaaacggggtctgtgctgcgtactcttgctttgtggagctgtgttcgtgtcaccttcttacgaggtgagaaatgtatccggagtctaccatgtaacgaacgactgctctaacgccagtatcgtatacgaggcggccgacatgattatgcacaccccgggctgcgtcccttgtgttcgggaaaataatagtagcagatgctgggtcgcgttgacgccaaccctcgcagcgcgaaacgcatcagttccgacgacgactatccgcagacatgttgatctcttggtgggagcggctgcgttgtgtagtgcaatgtatgtcggagatctttgtgggtctgtcttccttgtcgcccaactgttcacgttttccccaagacgccatgaaacggtacaagattgtaattgttcaatatacccgggacacgtcacgggacacaggatggcctgggatatgatgatgaattggagtccaaccgcagctttggtcgtaagtcaacttttgcgaatcccccaggccgtggttgacatggtagcgggcgcgcactggggagtgcttgcgggactcgcatattacagtatggttgggaattgggcgaaggtcttgattgtgatgttgctgtttgctggagtcgacggtggtacgtacgtgaccgggggcaccatggccaagaacaccctcgggattacctctcttttttccccgggctcatcccaaaaaatacaacttgtgaacacgaacggctcttggcatattaatcgaacagcattgaactgtaatgactccctcaacacggggttcttggcggcattgttttatgtgcacaagtttaacagctccggctgccccgaacggatggcgagttgttccccaatcgacgccttcgctcaaggatgggggccgattacatataatgaaagtcatagtagcgatcagaggccgtactgctggcactacgcgccaagaccatgcggcatcgtccctgcagcacaagtctgcgggcctgtttattgcttcactccgagccccgtagttgtgggcactactgacaggttcggagtgccgacttatagttggggggagaatgagaccgacgtcctcttgctcaataacaccaggcctccgcagggtaattggttcggctgtacttggatgaatagcacggggtttaccaaaacatgcggaggacccccctgtaacattggaggaataggtaacaaaaccctgacatgtccgacggattgcttccgcaaacatccagaggcaacctacactaaatgtggtagtgggccttggctgacgccacgctgtcttgttcactatccgtatagactgtggcattatccgtgcactgtcaactttacgatcttcaaagtgaggatgtatgtcgggggcgttgaacacagacttgaagcggcctgcaattggactcgaggcgaacgctgtaatcttgaagaccgggaccgatctgagttgtcacctcttcttttgagcaccactgagtggcaagtgctgccatgcagcttcacaacactcccagcactctcaaccgggcttatccacttgcaccaaaacgtggtcgatgtccaatatttgtatgggatcggcagcgcagttgtgagtttcgccattaagtgggaatatgtactccttctgttcctgctcttggcagacgcgcgagtctgcgcatgcttgtggatgatgctcctcattgctcaagcagaggcataggtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtga

SEQ ID NO: 81 - DNA, 1761 bp, synthetic construct
  source 1..1761: mol_type = other DNA
gccaccatggacgcaatgaaacggggtctgtgctgcgtactcttgctttgtggagctgtgttcgtgtcaccttctctggaatggcgaaacacatcaggcctgtacgtattgaccaacgattgcagcaactcctctatagtctatgaagcggacgatgtcatattgcacaccccgggatgtgtaccttgtgtacaggatgacaatacgtcaacttgttggacgccggttactccgactgtagcagtccgctacgtcggagctacaacagcgagtatacgctcacatgtagatttgctggtaggtgcagcaaccctctgtagcgctctgtatgttggcgacatgtgcggggcagtttttcttgtggggcaggcctttacttttagacccagaagacaccaaactgtgcaaacttgtaattgtagcctttaccctggccatgtcagcggtcacagaatggcctgggatatgatgatgaactggtcacccgctgtgggtatggtcgttgctcacatcctgcgacttccacagactcttttcgatatccttgctggtgcgcactgggggatacttgctggtctggcctactattcaatgcaagggaactgggcaaaagtggccatcgtcatgattatgttttctggagtcgatgcggagacatatgtcacgggggggtctgtggcgcattcagcacgcggactgacatcattgttctctatgggggccaaacaaaaactgcaactcgttaataccaacggctcatggcacattaacagcaccgccctcaactgtaatgaatccattaacacggggtttattgcagggttgttttattaccacaaatttaacagtacagggtgcccacagaggctgtctagttgccgccctataatcagctttagacagggatggggccctttgactgacgccaatattacgggtccttccgacgatagaccctactgttggcattatgccccgcgaccgtgcggtgtcgtacctgcttcatctgtctgtggcccggtttattgctttactccaagccccgtcgtggtgggcacgacagatatcaaaggacgacctacgtataattggggcgaaaatgaaactgatgtatttcttctggaaagtcttcgaccgccaagtggtcggtggtttggctgtgcctggatgaatagtactgggttccttaaaacgtgtggcgcgcctccttgtaacatttacggtggggaaggcgatcctgagaatgaaacagacttgttctgtcctacggactgttttagaaagcacccggaagccacctattcaaggtgcggtgcgggaccgtggttgaccccaaggtgcatggtggactatccttaccggctgtggcattacccgtgcaccgtaaattttactcttttcaaagttaggatgtttgttggaggctttgaacatcggtttactgcagcttgtaactggacgcgcggggagcgctgtaacattgaggatagggataggtctgaacaacatccgttgctgcattcaacaacagaacttgcgatccttccatgtagtttcacacccatgcccgctttgtcaacaggtctcatccaccttcaccaaaatatagttgacgtgcagtatctctacggcgtgggtagtgatatggttggatgggcactgaaatgggaatttgtcatattggtattccttctgctggcagacgctcgagtatgcgtagccctttggctcatgctgatgatatctcaggcggaggcctag

SEQ ID NO: 82 - DNA, 1755 bp, synthetic construct
  source 1..1755: mol_type = other DNA
gccaccatggacgcaatgaaacggggtctgtgctgcgtactcttgctttgtggagctgtgttcgtgtcaccttctgcggaggtcaaaaacatctctacgggttatatggttactaacgactgcacgaatgattctataacgtggcaacttcaagcagccgtattgcatgttcctggatgcgtaccttgcgagaaggttgggaatgcttctcaatgctggattcccgttagccccaacgtagcggtgcaaagacctggggcccttacccagggcctcagaacccacatcgatatggttgtaatgagcgcaacactttgttccgcactttatgttggagacttgtgcggtggtgtaatgttggctgcgcaaatgttcattgtaagccctcagcatcattggttcgttcaagactgcaactgttccatttaccccgggactatcactggtcacaggatggcgtgggacatgatgatgaactggagccctaccgcgaccatgatactggcgtacgctatgcgcgtgcccgaagttattattgatattatctccggcgcacattggggagttatgtttggcctggcatatttctctatgcaaggtgcgtgggcaaaggtagttgtcatacttttgttggcagcaggagtagatgcgcgaacccacaccgtcggagggtcagccgcacaaactaccggaaggttgacgagtttgtttgatatgggcccacgacaaaagattcaattggttaatacgaacgggtcctggcatattaacagaactgcacttaactgcaatgactcccttcatactggattcatcgcaagtcttttctatacacacagttttaacagctcaggctgtcccgagaggatgagcgcatgccgctccatcgaggcctttagggtaggttggggagcgctgcagtacgaagacaatgtgaccaatcccgaagacatgcggccgtactgttggcactacccaccacgacaatgtggtgtagtttcagcgaaaacggtttgtggcccagtttactgctttaccccttcaccagtcgtggttgggaccaccgacaggctgggagcccctacctatacctggggtgaaaacgaaaccgatgtctttctcctcaactctacaaggccccccctgggatcctggttcggctgtacttggatgaattcctcagggtacacgaagacgtgtggagccccgccttgccgcacgagggcggattttaatgcgtccaccgaccttttgtgcccgactgactgttttcgcaagcaccccgatacgacttacctcaagtgtggttccggtccgtggctcacgccgcgatgcttgatagactatccttaccggctttggcactacccttgtactgtgaattatactatattcaagattcgcatgtacgtgggcggggttgaacataggttgactgcggcttgcaatttcactagaggagatagatgcaacctcgaggatcgggataggagtcaactgtcccctctcctccacagcacgacagagtgggccatactgccatgttcttactctgaccttccggcgttgtccacggggcttcttcacctgcatcagaatattgtagatgttcagttcatgtacggcctgtctcctgcgctcacaaaatatatcgttcgatgggagtgggttatcctcctcttccttctgctcgcggatgcgcgggtatgtgcgtgcctctggatgcttatactgcttggccaggcagaggcctag

SEQ ID NO: 83 - DNA, 3917 bp, synthetic construct
  source 1..3917: mol_type = other DNA
gccaccatggacgcaatgaaacggggtctgtgctgcgtactcttgctttgtggagctgtgttcgtgtcaccttcttacgaggtgagaaatgtatccggagtctaccatgtaacgaacgactgctctaacgccagtatcgtatacgaggcggccgacatgattatgcacaccccgggctgcgtcccttgtgttcgggaaaataatagtagcagatgctgggtcgcgttgacgccaaccctcgcagcgcgaaacgcatcagttccgacgacgactatccgcagacatgttgatctcttggtgggagcggctgcgttgtgtagtgcaatgtatgtcggagatctttgtgggtctgtcttccttgtcgcccaactgttcacgttttccccaagacgccatgaaacggtacaagattgtaattgttcaatatacccgggacacgtcacgggacacaggatggcctgggatatgatgatgaattggagtccaaccgcagctttggtcgtaagtcaacttttgcgaatcccccaggccgtggttgacatggtagcgggcgcgcactggggagtgcttgcgggactcgcatattacagtatggttgggaattgggcgaaggtcttgattgtgatgttgctgtttgctggagtcgacggtggtacgtacgtgaccgggggcaccatggccaagaacaccctcgggattacctctcttttttccccgggctcatcccaaaaaatacaacttgtgaacacgaacggctcttggcatattaatcgaacagcattgaactgtaatgactccctcaacacggggttcttggcggcattgttttatgtgcacaagtttaacagctccggctgccccgaacggatggcgagttgttccccaatcgacgccttcgctcaaggatgggggccgattacatataatgaaagtcatagtagcgatcagaggccgtactgctggcactacgcgccaagaccatgcggcatcgtccctgcagcacaagtctgcgggcctgtttattgcttcactccgagccccgtagttgtgggcactactgacaggttcggagtgccgacttatagttggggggagaatgagaccgacgtcctcttgctcaataacaccaggcctccgcagggtaattggttcggctgtacttggatgaatagcacggggtttaccaaaacatgcggaggacccccctgtaacattggaggaataggtaacaaaaccctgacatgtccgacggattgcttccgcaaacatccagaggcaacctacactaaatgtggtagtgggccttggctgacgccacgctgtcttgttcactatccgtatagactgtggcattatccgtgcactgtcaactttacgatcttcaaagtgaggatgtatgtcgggggcgttgaacacagacttgaagcggcctgcaattggactcgaggcgaacgctgtaatcttgaagaccgggaccgatctgagttgtcacctcttcttttgagcaccactgagtggcaagtgctgccatgcagcttcacaacactcccagcactctcaaccgggcttatccacttgcaccaaaacgtggtcgatgtccaatatttgtatgggatcggcagcgcagttgtgagtttcgccattaagtgggaatatgtactccttctgttcctgctcttggcagacgcgcgagtctgcgcatgcttgtggatgatgctcctcattgctcaagcagaggcataggtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcatggacgcaatgaaacggggtctgtgctgcgtactcttgctttgtggagctgtgttcgtgtcaccttctctggaatggcgaaacacatcaggcctgtacgtattgaccaacgattgcagcaactcctctatagtctatgaagcggacgatgtcatattgcacaccccgggatgtgtaccttgtgtacaggatgacaatacgtcaacttgttggacgccggttactccgactgtagcagtccgctacgtcggagctacaacagcgagtatacgctcacatgtagatttgctggtaggtgcagcaaccctctgtagcgctctgtatgttggcgacatgtgcggggcagtttttcttgtggggcaggcctttacttttagacccagaagacaccaaactgtgcaaacttgtaattgtagcctttaccctggccatgtcagcggtcacagaatggcctgggatatgatgatgaactggtcacccgctgtgggtatggtcgttgctcacatcctgcgacttccacagactcttttcgatatccttgctggtgcgcactgggggatacttgctggtctggcctactattcaatgcaagggaactgggcaaaagtggccatcgtcatgattatgttttctggagtcgatgcggagacatatgtcacgggggggtctgtggcgcattcagcacgcggactgacatcattgttctctatgggggccaaacaaaaactgcaactcgttaataccaacggctcatggcacattaacagcaccgccctcaactgtaatgaatccattaacacggggtttattgcagggttgttttattaccacaaatttaacagtacagggtgcccacagaggctgtctagttgccgccctataatcagctttagacagggatggggccctttgactgacgccaatattacgggtccttccgacgatagaccctactgttggcattatgccccgcgaccgtgcggtgtcgtacctgcttcatctgtctgtggcccggtttattgctttactccaagccccgtcgtggtgggcacgacagatatcaaaggacgacctacgtataattggggcgaaaatgaaactgatgtatttcttctggaaagtcttcgaccgccaagtggtcggtggtttggctgtgcctggatgaatagtactgggttccttaaaacgtgtggcgcgcctccttgtaacatttacggtggggaaggcgatcctgagaatgaaacagacttgttctgtcctacggactgttttagaaagcacccggaagccacctattcaaggtgcggtgcgggaccgtggttgaccccaaggtgcatggtggactatccttaccggctgtggcattacccgtgcaccgtaaattttactcttttcaaagttaggatgtttgttggaggctttgaacatcggtttactgcagcttgtaactggacgcgcggggagcgctgtaacattgaggatagggataggtctgaacaacatccgttgctgcattcaacaacagaacttgcgatccttccatgtagtttcacacccatgcccgctttgtcaacaggtctcatccaccttcaccaaaatatagttgacgtgcagtatctctacggcgtgggtagtgatatggttggatgggcactgaaatgggaatttgtcatattggtattccttctgctggcagacgctcgagtatgcgtagccctttggctcatgctgatgatatctcaggcggaggcctag

SEQ ID NO: 84 - DNA, 2415 bp, synthetic construct
  source 1..2415: mol_type = other DNA
gccaccatggacgcaatgaaacggggtctgtgctgcgtactcttgctttgtggagctgtgttcgtgtcaccttctgcggaggtcaaaaacatctctacgggttatatggttactaacgactgcacgaatgattctataacgtggcaacttcaagcagccgtattgcatgttcctggatgcgtaccttgcgagaaggttgggaatgcttctcaatgctggattcccgttagccccaacgtagcggtgcaaagacctggggcccttacccagggcctcagaacccacatcgatatggttgtaatgagcgcaacactttgttccgcactttatgttggagacttgtgcggtggtgtaatgttggctgcgcaaatgttcattgtaagccctcagcatcattggttcgttcaagactgcaactgttccatttaccccgggactatcactggtcacaggatggcgtgggacatgatgatgaactggagccctaccgcgaccatgatactggcgtacgctatgcgcgtgcccgaagttattattgatattatctccggcgcacattggggagttatgtttggcctggcatatttctctatgcaaggtgcgtgggcaaaggtagttgtcatacttttgttggcagcaggagtagatgcgcgaacccacaccgtcggagggtcagccgcacaaactaccggaaggttgacgagtttgtttgatatgggcccacgacaaaagattcaattggttaatacgaacgggtcctggcatattaacagaactgcacttaactgcaatgactcccttcatactggattcatcgcaagtcttttctatacacacagttttaacagctcaggctgtcccgagaggatgagcgcatgccgctccatcgaggcctttagggtaggttggggagcgctgcagtacgaagacaatgtgaccaatcccgaagacatgcggccgtactgttggcactacccaccacgacaatgtggtgtagtttcagcgaaaacggtttgtggcccagtttactgctttaccccttcaccagtcgtggttgggaccaccgacaggctgggagcccctacctatacctggggtgaaaacgaaaccgatgtctttctcctcaactctacaaggccccccctgggatcctggttcggctgtacttggatgaattcctcagggtacacgaagacgtgtggagccccgccttgccgcacgagggcggattttaatgcgtccaccgaccttttgtgcccgactgactgttttcgcaagcaccccgatacgacttacctcaagtgtggttccggtccgtggctcacgccgcgatgcttgatagactatccttaccggctttggcactacccttgtactgtgaattatactatattcaagattcgcatgtacgtgggcggggttgaacataggttgactgcggcttgcaatttcactagaggagatagatgcaacctcgaggatcgggataggagtcaactgtcccctctcctccacagcacgacagagtgggccatactgccatgttcttactctgaccttccggcgttgtccacggggcttcttcacctgcatcagaatattgtagatgttcagttcatgtacggcctgtctcctgcgctcacaaaatatatcgttcgatgggagtgggttatcctcctcttccttctgctcgcggatgcgcgggtatgtgcgtgcctctggatgcttatactgcttggccaggcagaggcctagcttggtttctggagacaggctaaggatgcccttcaggtaccccgaggtaacaagcgacactcgggatctgagaaggggattggggcttctatccaagcgcccaatttaaaaagcttctatgcctgaataggtgaccggaggccggcaccttttcctttacccacaacttactttatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtga

SEQ ID NO: 85 - DNA, 2315 bp, synthetic construct
  source 1..2315: mol_type = other DNA
gccaccatggacgcaatgaaacggggtctgtgctgcgtactcttgctttgtggagctgtgttcgtgtcaccttcttaccaggtgaggaattcatccggactgtaccatgtgacaaatgattgcccgaacagtagcatcgtctatgaggcggcggatgcgatactccatacgccagggtgtgtaccatgtgtaagggaaggaaatgctagtcgatgctgggttgcagtcacacctacagtggccacacgagacggcaaattgccaactactcagttgcggcgccacatagatctcttggtcggtagtgctacactgtgctctgccctgtacgtcggagacctctgtggatcagtttttctcgtggggcaactttttacattttcacctcgccggcattggaccacacaggactgtaactgttccatctatccaggccatattaccggtcatagaatggcatgggacatgatgatgaattggtctccgactgctgcattggtcgtagcccaactgctgaggatcccccaagccattatggatatgatagcgggggcacactggggggtgctcgccgggatcgcttacttctctatggtagggaactgggccaaggttctggtggtcctgctgctttttgcaggggtggatgccgagacgcatgtgacgggtgggaatgcaggtcgcaccactgctggtctcgtggggctgttgactcccggagcgaagcagaatattcagcttattaacacaaacgggtcctggcacattaattctacagcgctgaactgcaatgaaagtctcaatacgggttggctcgcggggttgttttatcaacacaagtttaactcctcaggatgtccagagcgcctcgcttcctgcagaagattgacggatttcgctcagggatgggggcccatctcatatgcaaacggttcaggccttgacgaaagaccatactgttggcactatccacctcgaccgtgtggaatcgtgccagcgaaatctgtctgtgggccagtctactgctttaccccatcacccgttgtagtgggaaccactgacagaagtggggcccccacctacagttggggggctaacgataccgacgtattcgtcctgaataatacccggccaccgcttggtaactggttcggttgtacctggatgaacagtacagggttcaccaaggtttgcggtgctccaccgtgcgtgataggtggcgttgggaataacacgctgctctgcccaaccgactgtttccgaaagcacccggaggcgacgtattcaaggtgcgggtcagggccctggatcactccccgctgtatggtagattacccgtacaggctgtggcactatccttgtactatcaattacaccatcttcaaagtacggatgtatgtggggggcgtcgagcaccgacttgaagctgcatgtaattggactcggggggagcgatgtgacctggaggatagggaccgcagcgaactctcacctctcctgctttctaccactcagtggcaagtcctgccctgtagtttcacgacattgccggcattgtcaactggtttgatccacctgcatcaaaacatcgttgacgtgcaatacctgtatggcgtaggcagcagtatcgcgtcctgggcgataaaatgggagtacgttgtactcctgtttttgttgctggccgatgcgcgagtttgcagttgcttgtggatgatgcttctcatttcacaagcggaataggtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctacccctag

SEQ ID NO: 86 - DNA, 2028 bp, synthetic construct
  source 1..2028: mol_type = other DNA
gccaccatggacgcaatgaaacggggtctgtgctgcgtactcttgctttgtggagctgtgttcgtgtcaccttcttacgaggtgagaaatgtatccggagtctaccatgtaacgaacgactgctctaacgccagtatcgtatacgaggcggccgacatgattatgcacaccccgggctgcgtcccttgtgttcgggaaaataatagtagcagatgctgggtcgcgttgacgccaaccctcgcagcgcgaaacgcatcagttccgacgacgactatccgcagacatgttgatctcttggtgggagcggctgcgttgtgtagtgcaatgtatgtcggagatctttgtgggtctgtcttccttgtcgcccaactgttcacgttttccccaagacgccatgaaacggtacaagattgtaattgttcaatatacccgggacacgtcacgggacacaggatggcctgggatatgatgatgaattggagtccaaccgcagctttggtcgtaagtcaacttttgcgaatcccccaggccgtggttgacatggtagcgggcgcgcactggggagtgcttgcgggactcgcatattacagtatggttgggaattgggcgaaggtcttgattgtgatgttgctgtttgctggagtcgacggtggtacgtacgtgaccgggggcaccatggccaagaacaccctcgggattacctctcttttttccccgggctcatcccaaaaaatacaacttgtgaacacgaacggctcttggcatattaatcgaacagcattgaactgtaatgactccctcaacacggggttcttggcggcattgttttatgtgcacaagtttaacagctccggctgccccgaacggatggcgagttgttccccaatcgacgccttcgctcaaggatgggggccgattacatataatgaaagtcatagtagcgatcagaggccgtactgctggcactacgcgccaagaccatgcggcatcgtccctgcagcacaagtctgcgggcctgtttattgcttcactccgagccccgtagttgtgggcactactgacaggttcggagtgccgacttatagttggggggagaatgagaccgacgtcctcttgctcaataacaccaggcctccgcagggtaattggttcggctgtacttggatgaatagcacggggtttaccaaaacatgcggaggacccccctgtaacattggaggaataggtaacaaaaccctgacatgtccgacggattgcttccgcaaacatccagaggcaacctacactaaatgtggtagtgggccttggctgacgccacgctgtcttgttcactatccgtatagactgtggcattatccgtgcactgtcaactttacgatcttcaaagtgaggatgtatgtcgggggcgttgaacacagacttgaagcggcctgcaattggactcgaggcgaacgctgtaatcttgaagaccgggaccgatctgagttgtcacctcttcttttgagcaccactgagtggcaagtgctgccatgcagcttcacaacactcccagcactctcaaccgggcttatccacttgcaccaaaacgtggtcgatgtccaatatttgtatgggatcggcagcgcagttgtgagtttcgccattaagtgggaatatgtactccttctgttcctgctcttggcagacgcgcgagtctgcgcatgcttgtggatgatgctcctcattgctcaagcagaggcatagcttggtttctggagacaggctaaggatgcccttcaggtaccccgaggtaacaagcgacactcgggatctgagaaggggattggggcttctatccaagcgcccaatttaaaaagcttctatgcctgaataggtgaccggaggccggcaccttttcctttacccacaacttactttatgaaatctaccaaggtgcctgccgcttacgccgcccagggctacaaggtcctggtgctgaaccccagcgtggccgctacactgggattcggcgcctacatgagcaagtag

SEQ ID NO: 87 - DNA, 2195 bp, synthetic construct
  source 1..2195: mol_type = other DNA
gccaccatggacgcaatgaaacggggtctgtgctgcgtactcttgctttgtggagctgtgttcgtgtcaccttctgcggaggtcaaaaacatctctacgggttatatggttactaacgactgcacgaatgattctataacgtggcaacttcaagcagccgtattgcatgttcctggatgcgtaccttgcgagaaggttgggaatgcttctcaatgctggattcccgttagccccaacgtagcggtgcaaagacctggggcccttacccagggcctcagaacccacatcgatatggttgtaatgagcgcaacactttgttccgcactttatgttggagacttgtgcggtggtgtaatgttggctgcgcaaatgttcattgtaagccctcagcatcattggttcgttcaagactgcaactgttccatttaccccgggactatcactggtcacaggatggcgtgggacatgatgatgaactggagccctaccgcgaccatgatactggcgtacgctatgcgcgtgcccgaagttattattgatattatctccggcgcacattggggagttatgtttggcctggcatatttctctatgcaaggtgcgtgggcaaaggtagttgtcatacttttgttggcagcaggagtagatgcgcgaacccacaccgtcggagggtcagccgcacaaactaccggaaggttgacgagtttgtttgatatgggcccacgacaaaagattcaattggttaatacgaacgggtcctggcatattaacagaactgcacttaactgcaatgactcccttcatactggattcatcgcaagtcttttctatacacacagttttaacagctcaggctgtcccgagaggatgagcgcatgccgctccatcgaggcctttagggtaggttggggagcgctgcagtacgaagacaatgtgaccaatcccgaagacatgcggccgtactgttggcactacccaccacgacaatgtggtgtagtttcagcgaaaacggtttgtggcccagtttactgctttaccccttcaccagtcgtggttgggaccaccgacaggctgggagcccctacctatacctggggtgaaaacgaaaccgatgtctttctcctcaactctacaaggccccccctgggatcctggttcggctgtacttggatgaattcctcagggtacacgaagacgtgtggagccccgccttgccgcacgagggcggattttaatgcgtccaccgaccttttgtgcccgactgactgttttcgcaagcaccccgatacgacttacctcaagtgtggttccggtccgtggctcacgccgcgatgcttgatagactatccttaccggctttggcactacccttgtactgtgaattatactatattcaagattcgcatgtacgtgggcggggttgaacataggttgactgcggcttgcaatttcactagaggagatagatgcaacctcgaggatcgggataggagtcaactgtcccctctcctccacagcacgacagagtgggccatactgccatgttcttactctgaccttccggcgttgtccacggggcttcttcacctgcatcagaatattgtagatgttcagttcatgtacggcctgtctcctgcgctcacaaaatatatcgttcgatgggagtgggttatcctcctcttccttctgctcgcggatgcgcgggtatgtgcgtgcctctggatgcttatactgcttggccaggcagaggcctagttcgataccaagagctggtgtctgagggattacgactcgcgtggtcggacagcacatgttagtcatgtattatggtgtttcccaacacgtatctttgctctagtattgaactttagtgatgagtgtacttggcatgtacacctgattagttagcccttgtggatacgccactgccgctcaggtacagtaggacataccccaaatattgggttccactcgtagagaagaaaattagtggcgaaacgtacggtttatgtcaaaggagaaggtgattttaattgcacctgaagcaaccctaggtcttccctttgatgtgagctaataaggagtaaccgattcttacaatgtgatcaatgctggaacagatcaagggcggcagacacctgatcttctgtcacagcaagaaaaagtgcgacgagctggccgccaagctgcggtga

SEQ ID NO: 88 - DNA, 2005 bp, synthetic construct
  source 1..2005: mol_type = other DNA
gccaccatggacgcaatgaaacggggtctgtgctgcgtactcttgctttgtggagctgtgttcgtgtcaccttctctggaatggcgaaacacatcaggcctgtacgtattgaccaacgattgcagcaactcctctatagtctatgaagcggacgatgtcatattgcacaccccgggatgtgtaccttgtgtacaggatgacaatacgtcaacttgttggacgccggttactccgactgtagcagtccgctacgtcggagctacaacagcgagtatacgctcacatgtagatttgctggtaggtgcagcaaccctctgtagcgctctgtatgttggcgacatgtgcggggcagtttttcttgtggggcaggcctttacttttagacccagaagacaccaaactgtgcaaacttgtaattgtagcctttaccctggccatgtcagcggtcacagaatggcctgggatatgatgatgaactggtcacccgctgtgggtatggtcgttgctcacatcctgcgacttccacagactcttttcgatatccttgctggtgcgcactgggggatacttgctggtctggcctactattcaatgcaagggaactgggcaaaagtggccatcgtcatgattatgttttctggagtcgatgcggagacatatgtcacgggggggtctgtggcgcattcagcacgcggactgacatcattgttctctatgggggccaaacaaaaactgcaactcgttaataccaacggctcatggcacattaacagcaccgccctcaactgtaatgaatccattaacacggggtttattgcagggttgttttattaccacaaatttaacagtacagggtgcccacagaggctgtctagttgccgccctataatcagctttagacagggatggggccctttgactgacgccaatattacgggtccttccgacgatagaccctactgttggcattatgccccgcgaccgtgcggtgtcgtacctgcttcatctgtctgtggcccggtttattgctttactccaagccccgtcgtggtgggcacgacagatatcaaaggacgacctacgtataattggggcgaaaatgaaactgatgtatttcttctggaaagtcttcgaccgccaagtggtcggtggtttggctgtgcctggatgaatagtactgggttccttaaaacgtgtggcgcgcctccttgtaacatttacggtggggaaggcgatcctgagaatgaaacagacttgttctgtcctacggactgttttagaaagcacccggaagccacctattcaaggtgcggtgcgggaccgtggttgaccccaaggtgcatggtggactatccttaccggctgtggcattacccgtgcaccgtaaattttactcttttcaaagttaggatgtttgttggaggctttgaacatcggtttactgcagcttgtaactggacgcgcggggagcgctgtaacattgaggatagggataggtctgaacaacatccgttgctgcattcaacaacagaacttgcgatccttccatgtagtttcacacccatgcccgctttgtcaacaggtctcatccaccttcaccaaaatatagttgacgtgcagtatctctacggcgtgggtagtgatatggttggatgggcactgaaatgggaatttgtcatattggtattccttctgctggcagacgctcgagtatgcgtagccctttggctcatgctgatgatatctcaggcggaggcctagcgggcccctgccgcctgcaagtcggaaattgcgctgtgctcctgtgctacggcctgtggctggactgcctgctgctgcccaactggctggcaagatgatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtga

SEQ ID NO: 89 - DNA, 980 bp, synthetic construct
  source 1..980: mol_type = other DNA
gccaccatggacgcaatgaaacggggtctgtgctgcgtactcttgctttgtggagctgtgttcgtgtcaccttctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtga

SEQ ID NO: 90 - DNA, 735 bp, synthetic construct
  source 1..735: mol_type = other DNA
gccaccatggacgcaatgaaacggggtctgtgctgcgtactcttgctttgtggagctgtgttcgtgtcaccttctcttggtttctggagacaggctaaggatgcccttcaggtaccccgaggtaacaagcgacactcgggatctgagaaggggattggggcttctatccaagcgcccaatttaaaaagcttctatgcctgaataggtgaccggaggccggcaccttttcctttacccacaacttactttatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtga

SEQ ID NO: 91 - DNA, 2600 bp, synthetic construct
  source 1..2600: mol_type = other DNA
gccaccatggacgcaatgaaacggggtctgtgctgcgtactcttgctttgtggagctgtgttcgtgtcaccttctctggaatggcgaaacacatcaggcctgtacgtattgaccaacgattgcagcaactcctctatagtctatgaagcggacgatgtcatattgcacaccccgggatgtgtaccttgtgtacaggatgacaatacgtcaacttgttggacgccggttactccgactgtagcagtccgctacgtcggagctacaacagcgagtatacgctcacatgtagatttgctggtaggtgcagcaaccctctgtagcgctctgtatgttggcgacatgtgcggggcagtttttcttgtggggcaggcctttacttttagacccagaagacaccaaactgtgcaaacttgtaattgtagcctttaccctggccatgtcagcggtcacagaatggcctgggatatgatgatgaactggtcacccgctgtgggtatggtcgttgctcacatcctgcgacttccacagactcttttcgatatccttgctggtgcgcactgggggatacttgctggtctggcctactattcaatgcaagggaactgggcaaaagtggccatcgtcatgattatgttttctggagtcgatgcggagacatatgtcacgggggggtctgtggcgcattcagcacgcggactgacatcattgttctctatgggggccaaacaaaaactgcaactcgttaataccaacggctcatggcacattaacagcaccgccctcaactgtaatgaatccattaacacggggtttattgcagggttgttttattaccacaaatttaacagtacagggtgcccacagaggctgtctagttgccgccctataatcagctttagacagggatggggccctttgactgacgccaatattacgggtccttccgacgatagaccctactgttggcattatgccccgcgaccgtgcggtgtcgtacctgcttcatctgtctgtggcccggtttattgctttactccaagccccgtcgtggtgggcacgacagatatcaaaggacgacctacgtataattggggcgaaaatgaaactgatgtatttcttctggaaagtcttcgaccgccaagtggtcggtggtttggctgtgcctggatgaatagtactgggttccttaaaacgtgtggcgcgcctccttgtaacatttacggtggggaaggcgatcctgagaatgaaacagacttgttctgtcctacggactgttttagaaagcacccggaagccacctattcaaggtgcggtgcgggaccgtggttgaccccaaggtgcatggtggactatccttaccggctgtggcattacccgtgcaccgtaaattttactcttttcaaagttaggatgtttgttggaggctttgaacatcggtttactgcagcttgtaactggacgcgcggggagcgctgtaacattgaggatagggataggtctgaacaacatccgttgctgcattcaacaacagaacttgcgatccttccatgtagtttcacacccatgcccgctttgtcaacaggtctcatccaccttcaccaaaatatagttgacgtgcagtatctctacggcgtgggtagtgatatggttggatgggcactgaaatgggaatttgtcatattggtattccttctgctggcagacgctcgagtatgcgtagccctttggctcatgctgatgatatctcaggcggaggcctagttcgataccaagagctggtgtctgagggattacgactcgcgtggtcggacagcacatgttagtcatgtattatggtgtttcccaacacgtatctttgctctagtattgaactttagtgatgagtgtacttggcatgtacacctgattagttagcccttgtggatacgccactgccgctcaggtacagtaggacataccccaaatattgggttccactcgtagagaagaaaattagtggcgaaacgtacggtttatgtcaaaggagaaggtgattttaattgcacctgaagcaaccctaggtcttccctttgatgtgagctaataaggagtaaccgattcttacaatgtgatcaatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtga

SEQ ID NO: 92 - DNA, 6676 bp, synthetic construct
  source 1..6676: mol_type = other DNA
gccaccatggacgcaatgaaacggggtctgtgctgcgtactcttgctttgtggagctgtgttcgtgtcaccttcttaccaggtgaggaattcatccggactgtaccatgtgacaaatgattgcccgaacagtagcatcgtctatgaggcggcggatgcgatactccatacgccagggtgtgtaccatgtgtaagggaaggaaatgctagtcgatgctgggttgcagtcacacctacagtggccacacgagacggcaaattgccaactactcagttgcggcgccacatagatctcttggtcggtagtgctacactgtgctctgccctgtacgtcggagacctctgtggatcagtttttctcgtggggcaactttttacattttcacctcgccggcattggaccacacaggactgtaactgttccatctatccaggccatattaccggtcatagaatggcatgggacatgatgatgaattggtctccgactgctgcattggtcgtagcccaactgctgaggatcccccaagccattatggatatgatagcgggggcacactggggggtgctcgccgggatcgcttacttctctatggtagggaactgggccaaggttctggtggtcctgctgctttttgcaggggtggatgccgagacgcatgtgacgggtgggaatgcaggtcgcaccactgctggtctcgtggggctgttgactcccggagcgaagcagaatattcagcttattaacacaaacgggtcctggcacattaattctacagcgctgaactgcaatgaaagtctcaatacgggttggctcgcggggttgttttatcaacacaagtttaactcctcaggatgtccagagcgcctcgcttcctgcagaagattgacggatttcgctcagggatgggggcccatctcatatgcaaacggttcaggccttgacgaaagaccatactgttggcactatccacctcgaccgtgtggaatcgtgccagcgaaatctgtctgtgggccagtctactgctttaccccatcacccgttgtagtgggaaccactgacagaagtggggcccccacctacagttggggggctaacgataccgacgtattcgtcctgaataatacccggccaccgcttggtaactggttcggttgtacctggatgaacagtacagggttcaccaaggtttgcggtgctccaccgtgcgtgataggtggcgttgggaataacacgctgctctgcccaaccgactgtttccgaaagcacccggaggcgacgtattcaaggtgcgggtcagggccctggatcactccccgctgtatggtagattacccgtacaggctgtggcactatccttgtactatcaattacaccatcttcaaagtacggatgtatgtggggggcgtcgagcaccgacttgaagctgcatgtaattggactcggggggagcgatgtgacctggaggatagggaccgcagcgaactctcacctctcctgctttctaccactcagtggcaagtcctgccctgtagtttcacgacattgccggcattgtcaactggtttgatccacctgcatcaaaacatcgttgacgtgcaatacctgtatggcgtaggcagcagtatcgcgtcctgggcgataaaatgggagtacgttgtactcctgtttttgttgctggccgatgcgcgagtttgcagttgcttgtggatgatgcttctcatttcacaagcggaataggtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcatggacgcaatgaaacggggtctgtgctgcgtactcttgctttgtggagctgtgttcgtgtcaccttctctggaatggcgaaacacatcaggcctgtacgtattgaccaacgattgcagcaactcctctatagtctatgaagcggacgatgtcatattgcacaccccgggatgtgtaccttgtgtacaggatgacaatacgtcaacttgttggacgccggttactccgactgtagcagtccgctacgtcggagctacaacagcgagtatacgctcacatgtagatttgctggtaggtgcagcaaccctctgtagcgctctgtatgttggcgacatgtgcggggcagtttttcttgtggggcaggcctttacttttagacccagaagacaccaaactgtgcaaacttgtaattgtagcctttaccctggccatgtcagcggtcacagaatggcctgggatatgatgatgaactggtcacccgctgtgggtatggtcgttgctcacatcctgcgacttccacagactcttttcgatatccttgctggtgcgcactgggggatacttgctggtctggcctactattcaatgcaagggaactgggcaaaagtggccatcgtcatgattatgttttctggagtcgatgcggagacatatgtcacgggggggtctgtggcgcattcagcacgcggactgacatcattgttctctatgggggccaaacaaaaactgcaactcgttaataccaacggctcatggcacattaacagcaccgccctcaactgtaatgaatccattaacacggggtttattgcagggttgttttattaccacaaatttaacagtacagggtgcccacagaggctgtctagttgccgccctataatcagctttagacagggatggggccctttgactgacgccaatattacgggtccttccgacgatagaccctactgttggcattatgccccgcgaccgtgcggtgtcgtacctgcttcatctgtctgtggcccggtttattgctttactccaagccccgtcgtggtgggcacgacagatatcaaaggacgacctacgtataattggggcgaaaatgaaactgatgtatttcttctggaaagtcttcgaccgccaagtggtcggtggtttggctgtgcctggatgaatagtactgggttccttaaaacgtgtggcgcgcctccttgtaacatttacggtggggaaggcgatcctgagaatgaaacagacttgttctgtcctacggactgttttagaaagcacccggaagccacctattcaaggtgcggtgcgggaccgtggttgaccccaaggtgcatggtggactatccttaccggctgtggcattacccgtgcaccgtaaattttactcttttcaaagttaggatgtttgttggaggctttgaacatcggtttactgcagcttgtaactggacgcgcggggagcgctgtaacattgaggatagggataggtctgaacaacatccgttgctgcattcaacaacagaacttgcgatccttccatgtagtttcacacccatgcccgctttgtcaacaggtctcatccaccttcaccaaaatatagttgacgtgcagtatctctacggcgtgggtagtgatatggttggatgggcactgaaatgggaatttgtcatattggtattccttctgctggcagacgctcgagtatgcgtagccctttggctcatgctgatgatatctcaggcggaggcctagcttggtttctggagacaggctaaggatgcccttcaggtaccccgaggtaacaagcgacactcgggatctgagaaggggattggggcttctatccaagcgcccaatttaaaaagcttctatgcctgaataggtgaccggaggccggcaccttttcctttacccacaacttactttatggacgcaatgaaacggggtctgtgctgcgtactcttgctttgtggagctgtgttcgtgtcaccttctgcggaggtcaaaaacatctctacgggttatatggttactaacgactgcacgaatgattctataacgtggcaacttcaagcagccgtattgcatgttcctggatgcgtaccttgcgagaaggttgggaatgcttctcaatgctggattcccgttagccccaacgtagcggtgcaaagacctggggcccttacccagggcctcagaacccacatcgatatggttgtaatgagcgcaacactttgttccgcactttatgttggagacttgtgcggtggtgtaatgttggctgcgcaaatgttcattgtaagccctcagcatcattggttcgttcaagactgcaactgttccatttaccccgggactatcactggtcacaggatggcgtgggacatgatgatgaactggagccctaccgcgaccatgatactggcgtacgctatgcgcgtgcccgaagttattattgatattatctccggcgcacattggggagttatgtttggcctggcatatttctctatgcaaggtgcgtgggcaaaggtagttgtcatacttttgttggcagcaggagtagatgcgcgaacccacaccgtcggagggtcagccgcacaaactaccggaaggttgacgagtttgtttgatatgggcccacgacaaaagattcaattggttaatacgaacgggtcctggcatattaacagaactgcacttaactgcaatgactcccttcatactggattcatcgcaagtcttttctatacacacagttttaacagctcaggctgtcccgagaggatgagcgcatgccgctccatcgaggcctttagggtaggttggggagcgctgcagtacgaagacaatgtgaccaatcccgaagacatgcggccgtactgttggcactacccaccacgacaatgtggtgtagtttcagcgaaaacggtttgtggcccagtttactgctttaccccttcaccagtcgtggttgggaccaccgacaggctgggagcccctacctatacctggggtgaaaacgaaaccgatgtctttctcctcaactctacaaggccccccctgggatcctggttcggctgtacttggatgaattcctcagggtacacgaagacgtgtggagccccgccttgccgcacgagggcggattttaatgcgtccaccgaccttttgtgcccgactgactgttttcgcaagcaccccgatacgacttacctcaagtgtggttccggtccgtggctcacgccgcgatgcttgatagactatccttaccggctttggcactacccttgtactgtgaattatactatattcaagattcgcatgtacgtgggcggggttgaacataggttgactgcggcttgcaatttcactagaggagatagatgcaacctcgaggatcgggataggagtcaactgtcccctctcctccacagcacgacagagtgggccatactgccatgttcttactctgaccttccggcgttgtccacggggcttcttcacctgcatcagaatattgtagatgttcagttcatgtacggcctgtctcctgcgctcacaaaatatatcgttcgatgggagtgggttatcctcctcttccttctgctcgcggatgcgcgggtatgtgcgtgcctctggatgcttatactgcttggccaggcagaggcctagttcgataccaagagctggtgtctgagggattacgactcgcgtggtcggacagcacatgttagtcatgtattatggtgtttcccaacacgtatctttgctctagtattgaactttagtgatgagtgtacttggcatgtacacctgattagttagcccttgtggatacgccactgccgctcaggtacagtaggacataccccaaatattgggttccactcgtagagaagaaaattagtggcgaaacgtacggtttatgtcaaaggagaaggtgattttaattgcacctgaagcaaccctaggtcttccctttgatgtgagctaataaggagtaaccgattcttacaatgtgatcaatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtga

SEQ ID NO: 93 - DNA, 6679 bp, synthetic construct
  source 1..6679: mol_type = other DNA
gccaccatggacgcaatgaaacggggtctgtgctgcgtactcttgctttgtggagctgtgttcgtgtcaccttcttacgaggtgagaaatgtatccggagtctaccatgtaacgaacgactgctctaacgccagtatcgtatacgaggcggccgacatgattatgcacaccccgggctgcgtcccttgtgttcgggaaaataatagtagcagatgctgggtcgcgttgacgccaaccctcgcagcgcgaaacgcatcagttccgacgacgactatccgcagacatgttgatctcttggtgggagcggctgcgttgtgtagtgcaatgtatgtcggagatctttgtgggtctgtcttccttgtcgcccaactgttcacgttttccccaagacgccatgaaacggtacaagattgtaattgttcaatatacccgggacacgtcacgggacacaggatggcctgggatatgatgatgaattggagtccaaccgcagctttggtcgtaagtcaacttttgcgaatcccccaggccgtggttgacatggtagcgggcgcgcactggggagtgcttgcgggactcgcatattacagtatggttgggaattgggcgaaggtcttgattgtgatgttgctgtttgctggagtcgacggtggtacgtacgtgaccgggggcaccatggccaagaacaccctcgggattacctctcttttttccccgggctcatcccaaaaaatacaacttgtgaacacgaacggctcttggcatattaatcgaacagcattgaactgtaatgactccctcaacacggggttcttggcggcattgttttatgtgcacaagtttaacagctccggctgccccgaacggatggcgagttgttccccaatcgacgccttcgctcaaggatgggggccgattacatataatgaaagtcatagtagcgatcagaggccgtactgctggcactacgcgccaagaccatgcggcatcgtccctgcagcacaagtctgcgggcctgtttattgcttcactccgagccccgtagttgtgggcactactgacaggttcggagtgccgacttatagttggggggagaatgagaccgacgtcctcttgctcaataacaccaggcctccgcagggtaattggttcggctgtacttggatgaatagcacggggtttaccaaaacatgcggaggacccccctgtaacattggaggaataggtaacaaaaccctgacatgtccgacggattgcttccgcaaacatccagaggcaacctacactaaatgtggtagtgggccttggctgacgccacgctgtcttgttcactatccgtatagactgtggcattatccgtgcactgtcaactttacgatcttcaaagtgaggatgtatgtcgggggcgttgaacacagacttgaagcggcctgcaattggactcgaggcgaacgctgtaatcttgaagaccgggaccgatctgagttgtcacctcttcttttgagcaccactgagtggcaagtgctgccatgcagcttcacaacactcccagcactctcaaccgggcttatccacttgcaccaaaacgtggtcgatgtccaatatttgtatgggatcggcagcgcagttgtgagtttcgccattaagtgggaatatgtactccttctgttcctgctcttggcagacgcgcgagtctgcgcatgcttgtggatgatgctcctcattgctcaagcagaggcataggtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcatggacgcaatgaaacggggtctgtgctgcgtactcttgctttgtggagctgtgttcgtgtcaccttctctggaatggcgaaacacatcaggcctgtacgtattgaccaacgattgcagcaactcctctatagtctatgaagcggacgatgtcatattgcacaccccgggatgtgtaccttgtgtacaggatgacaatacgtcaacttgttggacgccggttactccgactgtagcagtccgctacgtcggagctacaacagcgagtatacgctcacatgtagatttgctggtaggtgcagcaaccctctgtagcgctctgtatgttggcgacatgtgcggggcagtttttcttgtggggcaggcctttacttttagacccagaagacaccaaactgtgcaaacttgtaattgtagcctttaccctggccatgtcagcggtcacagaatggcctgggatatgatgatgaactggtcacccgctgtgggtatggtcgttgctcacatcctgcgacttccacagactcttttcgatatccttgctggtgcgcactgggggatacttgctggtctggcctactattcaatgcaagggaactgggcaaaagtggccatcgtcatgattatgttttctggagtcgatgcggagacatatgtcacgggggggtctgtggcgcattcagcacgcggactgacatcattgttctctatgggggccaaacaaaaactgcaactcgttaataccaacggctcatggcacattaacagcaccgccctcaactgtaatgaatccattaacacggggtttattgcagggttgttttattaccacaaatttaacagtacagggtgcccacagaggctgtctagttgccgccctataatcagctttagacagggatggggccctttgactgacgccaatattacgggtccttccgacgatagaccctactgttggcattatgccccgcgaccgtgcggtgtcgtacctgcttcatctgtctgtggcccggtttattgctttactccaagccccgtcgtggtgggcacgacagatatcaaaggacgacctacgtataattggggcgaaaatgaaactgatgtatttcttctggaaagtcttcgaccgccaagtggtcggtggtttggctgtgcctggatgaatagtactgggttccttaaaacgtgtggcgcgcctccttgtaacatttacggtggggaaggcgatcctgagaatgaaacagacttgttctgtcctacggactgttttagaaagcacccggaagccacctattcaaggtgcggtgcgggaccgtggttgaccccaaggtgcatggtggactatccttaccggctgtggcattacccgtgcaccgtaaattttactcttttcaaagttaggatgtttgttggaggctttgaacatcggtttactgcagcttgtaactggacgcgcggggagcgctgtaacattgaggatagggataggtctgaacaacatccgttgctgcattcaacaacagaacttgcgatccttccatgtagtttcacacccatgcccgctttgtcaacaggtctcatccaccttcaccaaaatatagttgacgtgcagtatctctacggcgtgggtagtgatatggttggatgggcactgaaatgggaatttgtcatattggtattccttctgctggcagacgctcgagtatgcgtagccctttggctcatgctgatgatatctcaggcggaggcctagcttggtttctggagacaggctaaggatgcccttcaggtaccccgaggtaacaagcgacactcgggatctgagaaggggattggggcttctatccaagcgcccaatttaaaaagcttctatgcctgaataggtgaccggaggccggcaccttttcctttacccacaacttactttatggacgcaatgaaacggggtctgtgctgcgtactcttgctttgtggagctgtgttcgtgtcaccttctgcggaggtcaaaaacatctctacgggttatatggttactaacgactgcacgaatgattctataacgtggcaacttcaagcagccgtattgcatgttcctggatgcgtaccttgcgagaaggttgggaatgcttctcaatgctggattcccgttagccccaacgtagcggtgcaaagacctggggcccttacccagggcctcagaacccacatcgatatggttgtaatgagcgcaacactttgttccgcactttatgttggagacttgtgcggtggtgtaatgttggctgcgcaaatgttcattgtaagccctcagcatcattggttcgttcaagactgcaactgttccatttaccccgggactatcactggtcacaggatggcgtgggacatgatgatgaactggagccctaccgcgaccatgatactggcgtacgctatgcgcgtgcccgaagttattattgatattatctccggcgcacattggggagttatgtttggcctggcatatttctctatgcaaggtgcgtgggcaaaggtagttgtcatacttttgttggcagcaggagtagatgcgcgaacccacaccgtcggagggtcagccgcacaaactaccggaaggttgacgagtttgtttgatatgggcccacgacaaaagattcaattggttaatacgaacgggtcctggcatattaacagaactgcacttaactgcaatgactcccttcatactggattcatcgcaagtcttttctatacacacagttttaacagctcaggctgtcccgagaggatgagcgcatgccgctccatcgaggcctttagggtaggttggggagcgctgcagtacgaagacaatgtgaccaatcccgaagacatgcggccgtactgttggcactacccaccacgacaatgtggtgtagtttcagcgaaaacggtttgtggcccagtttactgctttaccccttcaccagtcgtggttgggaccaccgacaggctgggagcccctacctatacctggggtgaaaacgaaaccgatgtctttctcctcaactctacaaggccccccctgggatcctggttcggctgtacttggatgaattcctcagggtacacgaagacgtgtggagccccgccttgccgcacgagggcggattttaatgcgtccaccgaccttttgtgcccgactgactgttttcgcaagcaccccgatacgacttacctcaagtgtggttccggtccgtggctcacgccgcgatgcttgatagactatccttaccggctttggcactacccttgtactgtgaattatactatattcaagattcgcatgtacgtgggcggggttgaacataggttgactgcggcttgcaatttcactagaggagatagatgcaacctcgaggatcgggataggagtcaactgtcccctctcctccacagcacgacagagtgggccatactgccatgttcttactctgaccttccggcgttgtccacggggcttcttcacctgcatcagaatattgtagatgttcagttcatgtacggcctgtctcctgcgctcacaaaatatatcgttcgatgggagtgggttatcctcctcttccttctgctcgcggatgcgcgggtatgtgcgtgcctctggatgcttatactgcttggccaggcagaggcctagttcgataccaagagctggtgtctgagggattacgactcgcgtggtcggacagcacatgttagtcatgtattatggtgtttcccaacacgtatctttgctctagtattgaactttagtgatgagtgtacttggcatgtacacctgattagttagcccttgtggatacgccactgccgctcaggtacagtaggacataccccaaatattgggttccactcgtagagaagaaaattagtggcgaaacgtacggtttatgtcaaaggagaaggtgattttaattgcacctgaagcaaccctaggtcttccctttgatgtgagctaataaggagtaaccgattcttacaatgtgatcaatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtga

SEQ ID NO: 94 - DNA, 419 bp, synthetic construct
  source 1..419: mol_type = other DNA
gtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttc

SEQ ID NO: 95 - DNA, 174 bp, synthetic construct
  source 1..174: mol_type = other DNA
cttggtttctggagacaggctaaggatgcccttcaggtaccccgaggtaacaagcgacactcgggatctgagaaggggattggggcttctatccaagcgcccaatttaaaaagcttctatgcctgaataggtgaccggaggccggcaccttttcctttacccacaacttacttt

SEQ ID NO: 96 - DNA, 432 bp, synthetic construct
  source 1..432: mol_type = other DNA
gtgcaatctgaaactccgcctggtctttccaggtctagaggggtgacactttgtactgtgcatgactccacgttcgatccactggcgagtgctagtagtagtactgctgtttcgtagcggaacacgacggccgtgggactccccccttggtaacaaggacccgcggggccaaaagccacgtctctgacccgtcgtgtgtgcaaacccagcacagcagctttactgcgaaaaccactttaaggtgacattgatactggtactcacgcactggtgacaggctaaggatgcccttcaggtaccccgaggcaacaagcgacactcgggatctgagaaggggactggggcttctgtaaaagcgcccagtttaaaaagcttctatgcctgaataggtgaccggaggccggcacctttccctctactaccactgactct

SEQ ID NO: 97 - DNA, 353 bp, synthetic construct
  source 1..353: mol_type = other DNA
ttcgataccaagagctggtgtctgagggattacgactcgcgtggtcggacagcacatgttagtcatgtattatggtgtttcccaacacgtatctttgctctagtattgaactttagtgatgagtgtacttggcatgtacacctgattagttagcccttgtggatacgccactgccgctcaggtacagtaggacataccccaaatattgggttccactcgtagagaagaaaattagtggcgaaacgtacggtttatgtcaaaggagaaggtgattttaattgcacctgaagcaaccctaggtcttccctttgatgtgagctaataaggagtaaccgattcttacaatgtgatca

SEQ ID NO: 98 - DNA, 174 bp, synthetic construct
  source 1..174: mol_type = other DNA
ccgggaacccctaccccatccccttattcagcacatgaaataaacaaggggcatccaaatcttgcggcaacgcccccgggacatgcatcgtcccctggactctctcaaaccccttatccctctggacagaatgcaggtccaaccacgctggtataccctcaaacccctcagaca

SEQ ID NO: 99 - DNA, 94 bp, synthetic construct
  source 1..94: mol_type = other DNA
cgggcccctgccgcctgcaagtcggaaattgcgctgtgctcctgtgctacggcctgtggctggactgcctgctgctgcccaactggctggcaag

SEQ ID NO: 100 - DNA, 294 bp, synthetic construct
  source 1..294: mol_type = other DNA
gctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaacc

SEQ ID NO: 101 - protein, 50 aa, synthetic construct
GVYLLPRRGPRLGVRATRKTSERSQPRGRRQPIPKARRSEGRSWAQPGYP

SEQ ID NO: 102 - protein, 35 aa, synthetic construct
KSTKVPAAYAAQGYKVLVLNPSVAATLGFGAYMSK

SEQ ID NO: 103 - protein, 27 aa, synthetic construct
LEQIKGGRHLIFCHSKKKCDELAAKLR

SEQ ID NO: 104 - protein, 48 aa, synthetic construct
ILRRHVGPGEGAVQWMNRLIAFASRGNHVSPTHYVPESDAAARVTQIL

SEQ ID NO: 105 - protein, 750 aa, synthetic construct
MSTNPKPQRLTKRNTVRRPQNVKFPGGGQIVGGVYLLPRRGPRLGVRGTRKSSERSQPRGRRQRIPKAASSQGKAWGKPGYPWPLYGNEGCGWAGWLLSPRGSRPTWGPTDPRHRSRNLGKVIDTMTCGFADLMGYIPVLGAPLGGVARALAHGVRVLEDGVNYATGNLPGCSFSIFLLALLSCLTVPASAYEVRNSSGVYHLTNDCPNASIVYETDNAILHEPGCVPCVREGNTSRCWEPVAPTLAVRYRGALTDDLRTHIDLVVASATLCSALYVGDICGAIFIASQAVLWKPGGGRIVQDCNCSIYPGHVTGHRMAWDMMQNWAPALSMVAAYAVRVPGVIITTVAGGHWGVLFGLAYFGMAGNWAKVILIMLLMSGVDAETMAVGARAAHTTGALVSLLNPGPSQRLQLINTNGSWHINRTALNCNDSLQTGFIAALFYTHRFNSSGCPERMASCKPLSDFDQGWGPLWYNSTERPSDQRPYCWHYAPSPCGIVPAKDVCGPVYCFTPSPVVVGTTDRRGVPTYTWGENESDVFLLNSTRPPQGSWFGCSWMNTTGFTKTCGGPPCKIRPQGAQSNTSLTCPTDCFRKHPRATYSACGSGPWLTPRCMVHYPYRLWHYPCTVNFTIHKVRLYIGGVEHRLDAACNWTRGERCDLEDRDRVDMSPLLHSTTELAILPCSFVPLPALSTGLIHLHQNIVDAQYLYGLSPAIISWAIRWEWVVLVFLLLADARICACLWMMMLMAQAEA

SEQ ID NO: 106 - DNA, 2235 bp, synthetic construct
  source 1..2235: mol_type = other DNA
atgtcaacaaatcctaaaccccagcgaaagacaaagagaaataccaacagacggccccaggatgtgaaattcccgggtggcggacagatcgttggcggagtctacctgcttccacggcgagggcccagactgggtgttagagcgacccgcaaaacgtcagagaggagccaaccccgcgggcggcgccagccgatacccaaagcaaggagacctgagggtagaacttgggcccaacccggctatccttggccactgtatggaaacgagggatgcgggtgggcgggatggctgctgtccccgcgcggatctagaccttcttgggggccaacggatccgagacggcgaagccggaatttgggcaaagttattgacactcttacttgcgggtttgctgatttgatgggatacatacctctcgtaggtgcgccactcggaggggctgctcgagctctcgctcatggcgtgagagtactcgaggacggtgtaaattatgctaccggcaatctgccaggctgctccttttccatattcctgttggccctcctgagctgcttgaccgtccctgcttccgcctaccaagtacgaaacagtagtggcctctaccatgtcacaaacgactgcccaaatagcagtatagtatacgaagcggcagacgccatccttcatactccgggatgtgtcccttgtgtgcgagagggtaacgcatcacgatgttgggtcgcggttacacccacagtcgcaacgcgagacggaaagttgccgacaactcagttgcgaaggcatatagatttgctggttggctctgctaccctttgtagcgcactctacgtcggagacctgtgcggatctgtatttctcgttggtcaactgtttacattcagcccaagacgacattggactacccaggactgtaactgctccatatatcctggccatatcactggccacaggatggcgtgggacatgatgatgaactggagtcccaccgccgccttggtcgttgcgcagctgttgcgcattccccaagctattatggatatgattgcgggagcgcactggggagtcctggcaggtatcgcttatttcagtatggtggggaactgggcaaaggtgttggtggtcttgttgttgtttgctggggttgacgccgaaacccacgttacgggcggtaacgccggaaggacgacggcgggtctcgtcggattgttgacgcccggagcgaaacaaaatattcaattgataaataccaatggctcctggcatattaactcaactgcgctcaattgtaatgaaagtctcaacaccggttggctcgcaggtctcttctaccagcataaatttaattcatctggctgtccagaacggcttgctagttgcaggcgactgaccgacttcgcacagggatggggtccaatttcttacgcaaatggaagtggactggacgaaagaccgtattgctggcattatcctcctcgcccctgcggtatcgtgccggccaaaagcgtctgcggtcccgtctactgctttactccttctccagtagtggtcggtaccacggacaggtctggggcccctacgtactcctggggagcaaatgatacagacgtattcgtccttaacaatacgcgacccccgcttggcaactggtttggatgcacctggatgaattcaaccggattcaccaaggtatgtggtgcgccaccttgtgtcatagggggcgttggaaacaacactctgctctgtcccaccgattgttttcgcaaacatcctgaggctacgtatagtcgctgcggaagcggcccttggattactccacgatgtatggttgactacccctatagactctggcattacccatgcaccataaattatacaatttttaaggttcggatgtacgtcggtggcgtggaacacagactggaagctgcttgcaattggactcgaggcgaacgatgcgatcttgaggatcgagataggagcgagctctctcccctgctgcttagtacgacacaatggcaagttttgccgtgctcctttactaccttgcccgcgttgagcactggacttattcacttgcaccagaatatcgtagacgtccaatatttgtacggtgtcggatccagcattgccagctgggctattaagtgggagtatgtcgtactcctctttctgctgcttgccgatgcaagagtatgcagttgtttgtggatgatgctcctgatatcacaggcagaa

SEQ ID NO: 107 - DNA, 2235 bp, synthetic construct
  source 1..2235: mol_type = other DNA
atgagcactaatcccaagccgcagcgcaagactaagcggaataccaatagacgaccgcaggacgtaaaatttcccggtggaggacagatagtagggggagtctatcttcttcccagacggggaccgagattgggcgtcagggctacgaggaaggcttccgagagatcacagcccaggggccgccggcagccgatcccgaaagccagaaggccggaggggagggcctgggcgcagcctggttatccctggccgctctatggaaacgagggtctcggttgggccggttggttgctctcccccaggggaagccggccttcttggggccccacggatccccgccggaggtctagaaacctcgggaaagtaatagacacgctgacatgtggattcgcggacttgatggggtacataccgctcgtaggtgctccgctcggcggcgccgctcgcgctcttgctcatggggttcgggtccttgaagacggcgtgaattacgccacagggaatcttccggggtgcagtttctcaatctttctcctggcactgcttagttgtctgacaattccggctagcgcgtacgaggtgcgcaatgttagtggaatttatcacgtcacgaatgattgctctaattcttctatcgtttacgaggctgccgatgtaattatgcatacaccgggctgcgtaccatgtgtacaggagggtaatagttcacggtgctgggtcgccctgaccccgacacttgctgcccgaaacgcaagcgtaccgacgaccaccattcgcaggcatgtagatctgcttgtcggaaccgccgcattttgctctgctatgtatgttggagacctctgcggttcaatcttcctcgtgagtcaactttttacgttcagtccaaggaggcatgaaacggtccaggactgcaattgttctatttacccgggacatgtgtctggacacagaatggcctgggacatgatgatgaattggagtccaacgaccgctttggtcgtcagtcaattgctgcgcattccgcaagcagtagtcgatatggtagctggggcccattggggagttctggccggtttggcgtattattcaatggtcgggaactgggcaaaagtccttatagttgcattgcttttcgcaggtgtggatggagagacgcatacgaccggtcgcgtcgcagggcatacgaccagtggattcacttcactgttctcaagcggagcctcccaaaaaatccaactcgttaatactaacggaagctggcatatcaatcgaacagcactgaattgtaacgattccctgcaaacaggtttttttgccgcattgttttacgcgcataaattcaacagcagcgggtgtcctgagcgaatggcttcttgccggcccatcgactggttcgcgcaaggatggggtcccataacatatacaaagcccaattcctcagaccagcgcccatattgctggcattatgccccaaggccctgtggagtcgtcccggcgtcacaggtatgtggacccgtatactgtttcacgccttctcccgttgtggtgggaactactgatcgcagtggggtgcctacgtattcatggggtgaaaacgaaactgatgtcatgctcttgaataacacacggcccccgcaaggcaattggttcggctgtacatggatgaactccacgggcttcaccaaaacttgcggcggcccaccatgcaatatcggcggcgttggtaaccggacactcatttgtcccacagactgtttccgcaaacatccagaggccacatacacgaagtgtggcagtgggccttggttgactccgagatgcttggtcgattatccgtatcgcttgtggcactacccttgtacattgaacttctcaatctttaaagtaaggatgtacgtcgggggtgtggaacatagacttaatgccgcatgtaattggactcggggtgaaaggtgcaatctggaggatcgggaccgaagtgagcttagtccgctcctcctcagtacgactgagtggcagattctgccttgcgcctttacaactctgccggccctgagtacaggattgatccatttgcatcagaatatcgtggatgttcaatatttgtatggagtcggtagcgctttcgtatcattcgcaattaagtgggaatatatactccttctgttcttgcttcttgcagacgcgagagtgtgcgcatgtctctggatgatgttgctgatagcgcaggcagaa

SEQ ID NO: 108 - DNA, 2250 bp, synthetic construct
  source 1..2250: mol_type = other DNA
atgagtacaaatccaaaaccccaaaggaagacaaaacgaaatacaaataggcgccctcaagatgttaagtttccgggaggtggccagatcgtcggtggggtgtatctcctcccacggagagggccgagactgggcgtgcgcgcgacgcgcaagacatctgaacggtcacaaccacgaggcagacgacagcccatacctaaggatcgacgatccacgggcaagtcctgggggaaaccggggtatccctggccgctttacggaaacgaaggcttggggtgggctggctggctcctgtctccaagaggcagccgaccttcttggggccccaatgacccgcgacaccggagtcggaatgtggggaaggtaatagatacattgacctgtggatttgctgacctcatggggtatatcccagtcgtgggcgcccccctgggtggtgtagcacgagccctggcgcatggggttagagtgctggaggatggagtgaatttcgccacgggaaatctccccggttgcagcttctcaatctttttgcttgcacttttgagctgcattactacaccagtttctgctgcggaggttaaaaatatctcaaccggctacatggtgacgaacgactgtacgaacgactccattacatggcagcttcaggctgcggttctccatgtgccggggtgcgtgccttgcgaaaaagtcgggaatgcctcacaatgctggattccagtttcacccaacgtagcagtgcaacgaccaggtgcactcactcaaggtctgagaacccacattgatatggttgtaatgtctgcaacactctgttccgcactttatgtaggcgatctttgtggtggggtcatgcttgcagcccagatgtttattgtgagccctcagcaccattggttcgtacaggattgcaattgttctatctatcccggtacgattaccggacaccgaatggcgtgggacatgatgatgaactggtcacctaccgcgactatgattctggcctacgctatgcgcgttccagaggttatcattgacataatatctggtgcccattggggggtgatgtttggccttgcgtatttcagtatgcagggggcctgggccaaagtggttgttatcctgcttctcgccgcgggagtggatgcaaggactcatactgtaggcggctcagccgctcaaacgacagggcgattgactagtctctttgatatggggcctcgccaaaagatacaactggttaataccaacgggagttggcacatcaacagaacagcgctgaattgcaatgactccttgcacacaggtttcatcgctagtctcttttacacgcacagctttaatagctcaggttgtcccgaacggatgagtgcatgcagatcaattgaggcatttcgggtgggctggggtgcgctgcagtatgaggacaatgttacgaaccccgaggatatgcgaccctactgttggcattatcctccgagacagtgcggggttgtaagtgcgaaaactgtgtgtggtccagtgtattgctttacaccgagtcccgtcgttgttggtactaccgacaggttgggcgctcctacctatacctggggcgaaaatgagaccgatgtttttctgttgaacagtacacgacctccactgggcagttggttcggttgcacttggatgaacagttctggatacacaaaaacgtgtggggcgccaccgtgtagaacgcgcgccgattttaacgcatcaacggacctgctttgtcctacagattgctttcgcaaacatcctgataccacgtacttgaagtgtgggtctggaccctggctcaccccgcgatgtctcatcgactatccctaccggttgtggcactacccatgtaccgtcaactacaccatcttcaagattcggatgtacgtagggggtgttgagcaccgcctcaccgctgcctgcaatttcactaggggggaccgatgtaatcttgaggatcgcgatagaagtcaattgagcccgctcttgcactcaactaccgagtgggctattttgccatgcagctattcagacttgccagccttgagcaccggcctcctgcatctgcatcaaaatatcgtcgacgtacagttcatgtatggactgtctcctgcactgactaaatatatcgtccgctgggagtgggtgatcctcctcttcttgctccttgctgatgcgagggtctgtgcctgcttgtggatgctgatcctgcttggacaagcggaagcc

SEQ ID NO: 109 - DNA, 2241 bp, synthetic construct
  source 1..2241: mol_type = other DNA
atgtctacaaacccaaaacctcagcggaagaccaagaggaacactaaccggagaccaatggacgtcaagttccctggcggtggacaaatagtcggcggtgtttacctcttgcctaggaggggtccccgacttggcgtgagggccactagaaagacgtcagaacgctcacagcccagaggacggcgacagccgatacccaaggcaaggaggcctgaggggagatcttgggcacagccgggctatccgtggccattgtatggaaacgaaggatgcgggtgggcgggttggttgctgagtccccgcgggtcacggccaagttgggggcctaacgaccccagacgacgctcaagaaatctgggaaaagtcatagatacccttacgtgtggatttgcggacttgatgggatacatccctctggttggagcgccagttggcggtgtcgcaagggcccttgcgcatggagtcagggcccttgaggatggcattaactatgctacggggaacctcccagggtgttcatttagtatatttcttcttgccctcctctcttgccttacggtccccgcttccgcggtcaactaccggaacgtttcaggaatctaccacgtgactaatgactgccctaactcttccatcgtatatgaggcggatcatcacattctccaccttcccgggtgcgttccttgcgtaagggaggggaatcaatccagatgttgggtagcactgactccgacggtagctgccccgtatatcggggcaccacttgaatccctgcgatcccacgtagacttgatggtgggagctgccacggtttgctccggactgtacataggtgacctttgtggagggctctttttggtaggtcaaatgttcagctttaggcctcgccgacactggacgacacaagattgtaattgctccatctatacgggccacataaccggacacaggatggcatgggacatgatgatgaattggagccccaccacaacacttgtcctggcacaagtcatgaggatacccactacacttgttgatctgcttagtggaggacattggggtgttcttgtcggggtggcttatttcagcatgcaggctaattgggcaaaggttattttggtattgtttctgtttgcaggtgttgacgccgaaacccatgtctcaggagcagcagtagggagaagtacggcaggcttggctaacctcttctcctcaggttccaaacagaacttgcaacttatcaattcaaacggaagctggcacatcaaccgaacggcgcttaattgcaatgatagtctgaacacgggatttcttgcctccctgttttatacacataagtttaattccagcgggtgcagcgaaaggcttgcgtgttgtaaaagtcttgattcctacggtcagggatggggacccctcggcgtagcaaacataagtggttcatcagacgacagaccttattgttggcactatgctcccaggccctgcggaatcgtccccgcctcatcagtgtgcggtcccgtgtactgctttaccccctcaccggtggttgttgggacgactgatcatgtgggagtaccgacctacacatggggcgagaatgaaaccgacgtgttcctgctgaattctacaaggccgcctcacggagcgtggtttgggtgcgtttggatgaactcaacaggctttacgaaaacgtgtggggctccgccatgtgaggttaatacgaataatggaacgtggcactgcccaaccgattgcttcagaaagcatcccgaaacaacttatgccaaatgcggatctggcccctggataacccctaggtgcctcatagattatccataccgcttgtggcatttcccatgcacagctaacttttccgtatttaatattcggacctttgtcggaggaatagaacacagaatgcaggcagcttgtaattggacccgaggcgaagtttgcggtctggaacatagagaccgcgttgagttgagtcccttgttgctgacgacaactgcttggcagatactgccgtgttcttttaccacgcttcctgcgttgagtaccggattgatacatttgcaccaaaatatagtagacgtccagtacctgtatggagtcggcagtgccgtagtatcatgggctttgaaatgggagtacgttgttttggcgttcttgcttcttgctgatgcgcgggtgagtgcctgtctgtggatgatgttcatggtgagtcaagttgaggctgcc

SEQ ID NO: 110 - RNA, 3143 nt, synthetic construct
  source 1..3143: mol_type = other RNA
atgtcaacaaatcctaaaccccagcgaaagacaaagagaaataccaacagacggccccaggatgtgaaattcccgggtggcggacagatcgttggcggagtctacctgcttccacggcgagggcccagactgggtgttagagcgacccgcaaaacgtcagagaggagccaaccccgcgggcggcgccagccgatacccaaagcaaggagacctgagggtagaacttgggcccaacccggctatccttggccactgtatggaaacgagggatgcgggtgggcgggatggctgctgtccccgcgcggatctagaccttcttgggggccaacggatccgagacggcgaagccggaatttgggcaaagttattgacactcttacttgcgggtttgctgatttgatgggatacatacctctcgtaggtgcgccactcggaggggctgctcgagctctcgctcatggcgtgagagtactcgaggacggtgtaaattatgctaccggcaatctgccaggctgctccttttccatattcctgttggccctcctgagctgcttgaccgtccctgcttccgcctaccaagtacgaaacagtagtggcctctaccatgtcacaaacgactgcccaaatagcagtatagtatacgaagcggcagacgccatccttcatactccgggatgtgtcccttgtgtgcgagagggtaacgcatcacgatgttgggtcgcggttacacccacagtcgcaacgcgagacggaaagttgccgacaactcagttgcgaaggcatatagatttgctggttggctctgctaccctttgtagcgcactctacgtcggagacctgtgcggatctgtatttctcgttggtcaactgtttacattcagcccaagacgacattggactacccaggactgtaactgctccatatatcctggccatatcactggccacaggatggcgtgggacatgatgatgaactggagtcccaccgccgccttggtcgttgcgcagctgttgcgcattccccaagctattatggatatgattgcgggagcgcactggggagtcctggcaggtatcgcttatttcagtatggtggggaactgggcaaaggtgttggtggtcttgttgttgtttgctggggttgacgccgaaacccacgttacgggcggtaacgccggaaggacgacggcgggtctcgtcggattgttgacgcccggagcgaaacaaaatattcaattgataaataccaatggctcctggcatattaactcaactgcgctcaattgtaatgaaagtctcaacaccggttggctcgcaggtctcttctaccagcataaatttaattcatctggctgtccagaacggcttgctagttgcaggcgactgaccgacttcgcacagggatggggtccaatttcttacgcaaatggaagtggactggacgaaagaccgtattgctggcattatcctcctcgcccctgcggtatcgtgccggccaaaagcgtctgcggtcccgtctactgctttactccttctccagtagtggtcggtaccacggacaggtctggggcccctacgtactcctggggagcaaatgatacagacgtattcgtccttaacaatacgcgacccccgcttggcaactggtttggatgcacctggatgaattcaaccggattcaccaaggtatgtggtgcgccaccttgtgtcatagggggcgttggaaacaacactctgctctgtcccaccgattgttttcgcaaacatcctgaggctacgtatagtcgctgcggaagcggcccttggattactccacgatgtatggttgactacccctatagactctggcattacccatgcaccataaattatacaatttttaaggttcggatgtacgtcggtggcgtggaacacagactggaagctgcttgcaattggactcgaggcgaacgatgcgatcttgaggatcgagataggagcgagctctctcccctgctgcttagtacgacacaatggcaagttttgccgtgctcctttactaccttgcccgcgttgagcactggacttattcacttgcaccagaatatcgtagacgtccaatatttgtacggtgtcggatccagcattgccagctgggctattaagtgggagtatgtcgtactcctctttctgctgcttgccgatgcaagagtatgcagttgtttgtggatgatgctcctgatatcacaggcagaataggtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 111 - RNA, 2913 nt, synthetic construct
  source 1..2913: mol_type = other RNA
atgagtacaaatccaaaaccccaaaggaagacaaaacgaaatacaaataggcgccctcaagatgttaagtttccgggaggtggccagatcgtcggtggggtgtatctcctcccacggagagggccgagactgggcgtgcgcgcgacgcgcaagacatctgaacggtcacaaccacgaggcagacgacagcccatacctaaggatcgacgatccacgggcaagtcctgggggaaaccggggtatccctggccgctttacggaaacgaaggcttggggtgggctggctggctcctgtctccaagaggcagccgaccttcttggggccccaatgacccgcgacaccggagtcggaatgtggggaaggtaatagatacattgacctgtggatttgctgacctcatggggtatatcccagtcgtgggcgcccccctgggtggtgtagcacgagccctggcgcatggggttagagtgctggaggatggagtgaatttcgccacgggaaatctccccggttgcagcttctcaatctttttgcttgcacttttgagctgcattactacaccagtttctgctgcggaggttaaaaatatctcaaccggctacatggtgacgaacgactgtacgaacgactccattacatggcagcttcaggctgcggttctccatgtgccggggtgcgtgccttgcgaaaaagtcgggaatgcctcacaatgctggattccagtttcacccaacgtagcagtgcaacgaccaggtgcactcactcaaggtctgagaacccacattgatatggttgtaatgtctgcaacactctgttccgcactttatgtaggcgatctttgtggtggggtcatgcttgcagcccagatgtttattgtgagccctcagcaccattggttcgtacaggattgcaattgttctatctatcccggtacgattaccggacaccgaatggcgtgggacatgatgatgaactggtcacctaccgcgactatgattctggcctacgctatgcgcgttccagaggttatcattgacataatatctggtgcccattggggggtgatgtttggccttgcgtatttcagtatgcagggggcctgggccaaagtggttgttatcctgcttctcgccgcgggagtggatgcaaggactcatactgtaggcggctcagccgctcaaacgacagggcgattgactagtctctttgatatggggcctcgccaaaagatacaactggttaataccaacgggagttggcacatcaacagaacagcgctgaattgcaatgactccttgcacacaggtttcatcgctagtctcttttacacgcacagctttaatagctcaggttgtcccgaacggatgagtgcatgcagatcaattgaggcatttcgggtgggctggggtgcgctgcagtatgaggacaatgttacgaaccccgaggatatgcgaccctactgttggcattatcctccgagacagtgcggggttgtaagtgcgaaaactgtgtgtggtccagtgtattgctttacaccgagtcccgtcgttgttggtactaccgacaggttgggcgctcctacctatacctggggcgaaaatgagaccgatgtttttctgttgaacagtacacgacctccactgggcagttggttcggttgcacttggatgaacagttctggatacacaaaaacgtgtggggcgccaccgtgtagaacgcgcgccgattttaacgcatcaacggacctgctttgtcctacagattgctttcgcaaacatcctgataccacgtacttgaagtgtgggtctggaccctggctcaccccgcgatgtctcatcgactatccctaccggttgtggcactacccatgtaccgtcaactacaccatcttcaagattcggatgtacgtagggggtgttgagcaccgcctcaccgctgcctgcaatttcactaggggggaccgatgtaatcttgaggatcgcgatagaagtcaattgagcccgctcttgcactcaactaccgagtgggctattttgccatgcagctattcagacttgccagccttgagcaccggcctcctgcatctgcatcaaaatatcgtcgacgtacagttcatgtatggactgtctcctgcactgactaaatatatcgtccgctgggagtgggtgatcctcctcttcttgctccttgctgatgcgagggtctgtgcctgcttgtggatgctgatcctgcttggacaagcggaagcctagcttggtttctggagacaggctaaggatgcccttcaggtaccccgaggtaacaagcgacactcgggatctgagaaggggattggggcttctatccaagcgcccaatttaaaaagcttctatgcctgaataggtgaccggaggccggcaccttttcctttacccacaacttactttatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 112 - RNA, 3098 nt, synthetic construct
  source 1..3098: mol_type = other RNA
atgagtactcttccaaagccccagagaaaaacgaagaggaatacaatcagacgcccacaggacgtgaagttcccagggggaggacagatcgttggtggagtatacgtacttccaagaagagggcctcgccttggcgttcgggctacccgaaagacgagtgagaggagccaacctcggggtcggcgccagccgatccccaaggctcggcggtccgaggggcggtcatgggcacagccgggttacccctggcctctttatggtaacgagggatgcggatgggctgggtggctcttgtctccccggggcagtcggccaagttggggaccaaatgatccccggcggcgctctcggaatctggggaaggtaatagacacacttacctgcggttttgcagatcttatgggatacatcccacttgtaggcgctccagtagggggtgtggcgcgcgccctcgcacatggtgtgcgggccttggaggatggtatcaattttgcgacgggcaatctgccaggatgttcattctccattttccttctcgctcttttctcttgtctcgtacaccccgcggcgagtttggaatggagaaacacgagtggtctttatgtcttgaccaatgattgttccaactctagcatcgtgtatgaggctgatgatgtgatcctccacacgcccgggtgtgtgccgtgcgtccaagacgataatacttccacttgctggactcccgtaactcctactgttgctgttcgatatgtcggtgcaacgacagcgtctattcggagccacgtggacctgttggttggagcagctactctttgcagcgctctttatgtcggggatatgtgcggagcggttttccttgttggtcaggcgtttacatttcgaccccgacgacatcaaacggtgcaaacatgcaattgcagtctctacccgggacatgtatctgggcacaggatggcctgggacatgatgatgaattggtcccccgccgtagggatggtagttgcgcatatactccgacttccccagacgctgttcgacattttggctggcgcacattggggtatacttgccggtttggcttattatagcatgcagggaaactgggcaaaagtggcgattgtgatgattatgttcagcggggtggatgcagagacatacgtcacggggggctcagtagcgcatagcgcccgggggcttaccagtctgttttcaatgggggcgaaacagaagctgcaacttgtaaacaccaacggtagttggcacatcaattctacggccctgaattgtaacgagagtattaacacgggctttatcgcggggctgttttattaccataagttcaattctaccggctgcccgcaacgactttcttcctgtaagccaatcattagtttccggcagggttggggccctctgaccgacgccaacattaccggtccgagcgacgaccgaccttattgttggcactatgcacctaggccgtgtagcgtggttcctgctagtagcgtatgcgggcccgtctattgttttacaccttctccggtagttgttgggactacagacatcaaaggcaaaccgacgtacaactggggcgaaaatgagacggatgtcttcctgctggagtccctgcggccacccagtggacgatggttcggatgcgcctggatgaactcaactggtttcctgaagacttgtggggctcccccgtgcaacatttatggaggtgagggcgacccagagaatgagacagacttgttttgtccgacagactgttttcggaagcatcccgaggctacgtacagtcgctgcggtgccggtccgtggctgacgccaagatgcatggtggattatccgtacagactttggcattatccttgtacggtcaactttactttgtttaaagttagaatgttcgtgggggggtttgaacatcggttcactgctgcctgtaactggacacgcggcgagcgatgtaacattgaagacagggacagaagtgaacagcatccgctgctccattctactaccgaactcgctatactgccatgctcattcacacctatgccagctctctccaccgggttgatccacctccaccaaaatatcgtagacgttcaatacctttacggtgttgggtccgacatggtcggctgggcgttgaaatgggaattcgttatactcgtctttctcttgctcgcggatgcccgagtttgcgtagcactttggcttatgcttatggtatcacaggccgaggcatagttcgataccaagagctggtgtctgagggattacgactcgcgtggtcggacagcacatgttagtcatgtattatggtgtttcccaacacgtatctttgctctagtattgaactttagtgatgagtgtacttggcatgtacacctgattagttagcccttgtggatacgccactgccgctcaggtacagtaggacataccccaaatattgggttccactcgtagagaagaaaattagtggcgaaacgtacggtttatgtcaaaggagaaggtgattttaattgcacctgaagcaaccctaggtcttccctttgatgtgagctaataaggagtaaccgattcttacaatgtgatcaatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 113 - RNA, 1505 nt, synthetic construct
  source 1..1505: mol_type = other RNA
atggacgctatgaaacgagggctgtgctgcgtactgcttctttgtggtgcggtctttgtttctccgagctaccaagtacgaaacagtagtggcctctaccatgtcacaaacgactgcccaaatagcagtatagtatacgaagcggcagacgccatccttcatactccgggatgtgtcccttgtgtgcgagagggtaacgcatcacgatgttgggtcgcggttacacccacagtcgcaacgcgagacggaaagttgccgacaactcagttgcgaaggcatatagatttgctggttggctctgctaccctttgtagcgcactctacgtcggagacctgtgcggatctgtatttctcgttggtcaactgtttacattcagcccaagacgacattggactacccaggactgtaactgctccatatatcctggccatatcactggccacaggatggcgtgggacatgatgatgaactggagtcccaccgccgccttggtcgttgcgcagctgttgcgcattccccaagctattatggatatgattgcgggagcgcactggggagtcctggcaggtatcgcttatttcagtatggtggggaactaggtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 114 - RNA, 1811 nt, synthetic construct
  source 1..1811: mol_type = other RNA
atggacgctatgaaacgagggctgtgctgcgtactgcttctttgtggtgcggtctttgtttctccgagcgaaacccacgttacgggcggtaacgccggaaggacgacggcgggtctcgtcggattgttgacgcccggagcgaaacaaaatattcaattgataaataccaatggctcctggcatattaactcaactgcgctcaattgtaatgaaagtctcaacaccggttggctcgcaggtctcttctaccagcataaatttaattcatctggctgtccagaacggcttgctagttgcaggcgactgaccgacttcgcacagggatggggtccaatttcttacgcaaatggaagtggactggacgaaagaccgtattgctggcattatcctcctcgcccctgcggtatcgtgccggccaaaagcgtctgcggtcccgtctactgctttactccttctccagtagtggtcggtaccacggacaggtctggggcccctacgtactcctggggagcaaatgatacagacgtattcgtccttaacaatacgcgacccccgcttggcaactggtttggatgcacctggatgaattcaaccggattcaccaaggtatgtggtgcgccaccttgtgtcatagggggcgttggaaacaacactctgctctgtcccaccgattgttttcgcaaacatcctgaggctacgtatagtcgctgcggaagcggcccttggattactccacgatgtatggttgactacccctatagactctggcattacccatgcaccataaattatacaatttttaaggttcggatgtacgtcggtggcgtggaacacagactggaagctgcttgcaattggactcgaggcgaacgatgcgatcttgaggatcgagataggagcgagtaggtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 115 - RNA, 2639 nt, synthetic construct
  source 1..2639: mol_type = other RNA
atggacgctatgaaacgagggctgtgctgcgtactgcttctttgtggtgcggtctttgtttctccgagctaccaagtacgaaacagtagtggcctctaccatgtcacaaacgactgcccaaatagcagtatagtatacgaagcggcagacgccatccttcatactccgggatgtgtcccttgtgtgcgagagggtaacgcatcacgatgttgggtcgcggttacacccacagtcgcaacgcgagacggaaagttgccgacaactcagttgcgaaggcatatagatttgctggttggctctgctaccctttgtagcgcactctacgtcggagacctgtgcggatctgtatttctcgttggtcaactgtttacattcagcccaagacgacattggactacccaggactgtaactgctccatatatcctggccatatcactggccacaggatggcgtgggacatgatgatgaactggagtcccaccgccgccttggtcgttgcgcagctgttgcgcattccccaagctattatggatatgattgcgggagcgcactggggagtcctggcaggtatcgcttatttcagtatggtggggaactgggcaaaggtgttggtggtcttgttgttgtttgctggggttgacgccgaaacccacgttacgggcggtaacgccggaaggacgacggcgggtctcgtcggattgttgacgcccggagcgaaacaaaatattcaattgataaataccaatggctcctggcatattaactcaactgcgctcaattgtaatgaaagtctcaacaccggttggctcgcaggtctcttctaccagcataaatttaattcatctggctgtccagaacggcttgctagttgcaggcgactgaccgacttcgcacagggatggggtccaatttcttacgcaaatggaagtggactggacgaaagaccgtattgctggcattatcctcctcgcccctgcggtatcgtgccggccaaaagcgtctgcggtcccgtctactgctttactccttctccagtagtggtcggtaccacggacaggtctggggcccctacgtactcctggggagcaaatgatacagacgtattcgtccttaacaatacgcgacccccgcttggcaactggtttggatgcacctggatgaattcaaccggattcaccaaggtatgtggtgcgccaccttgtgtcatagggggcgttggaaacaacactctgctctgtcccaccgattgttttcgcaaacatcctgaggctacgtatagtcgctgcggaagcggcccttggattactccacgatgtatggttgactacccctatagactctggcattacccatgcaccataaattatacaatttttaaggttcggatgtacgtcggtggcgtggaacacagactggaagctgcttgcaattggactcgaggcgaacgatgcgatcttgaggatcgagataggagcgagctctctcccctgctgcttagtacgacacaatggcaagttttgccgtgctcctttactaccttgcccgcgttgagcactggacttattcacttgcaccagaatatcgtagacgtccaatatttgtacggtgtcggatccagcattgccagctgggctattaagtgggagtatgtcgtactcctctttctgctgcttgccgatgcaagagtatgcagttgtttgtggatgatgctcctgatatcacaggcagaataggtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 116 - RNA, 1260 nt, synthetic construct
  source 1..1260: mol_type = other RNA
atggacgctatgaaacgagggctgtgctgcgtactgcttctttgtggtgcggtctttgtttctccgagcgcggaggttaaaaatatctcaaccggctacatggtgacgaacgactgtacgaacgactccattacatggcagcttcaggctgcggttctccatgtgccggggtgcgtgccttgcgaaaaagtcgggaatgcctcacaatgctggattccagtttcacccaacgtagcagtgcaacgaccaggtgcactcactcaaggtctgagaacccacattgatatggttgtaatgtctgcaacactctgttccgcactttatgtaggcgatctttgtggtggggtcatgcttgcagcccagatgtttattgtgagccctcagcaccattggttcgtacaggattgcaattgttctatctatcccggtacgattaccggacaccgaatggcgtgggacatgatgatgaactggtcacctaccgcgactatgattctggcctacgctatgcgcgttccagaggttatcattgacataatatctggtgcccattggggggtgatgtttggccttgcgtatttcagtatgcagggggcctagcttggtttctggagacaggctaaggatgcccttcaggtaccccgaggtaacaagcgacactcgggatctgagaaggggattggggcttctatccaagcgcccaatttaaaaagcttctatgcctgaataggtgaccggaggccggcaccttttcctttacccacaacttactttatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 117 - RNA, 1578 nt, synthetic construct
  source 1..1578: mol_type = other RNA
atggacgctatgaaacgagggctgtgctgcgtactgcttctttgtggtgcggtctttgtttctccgagcaggactcatactgtaggcggctcagccgctcaaacgacagggcgattgactagtctctttgatatggggcctcgccaaaagatacaactggttaataccaacgggagttggcacatcaacagaacagcgctgaattgcaatgactccttgcacacaggtttcatcgctagtctcttttacacgcacagctttaatagctcaggttgtcccgaacggatgagtgcatgcagatcaattgaggcatttcgggtgggctggggtgcgctgcagtatgaggacaatgttacgaaccccgaggatatgcgaccctactgttggcattatcctccgagacagtgcggggttgtaagtgcgaaaactgtgtgtggtccagtgtattgctttacaccgagtcccgtcgttgttggtactaccgacaggttgggcgctcctacctatacctggggcgaaaatgagaccgatgtttttctgttgaacagtacacgacctccactgggcagttggttcggttgcacttggatgaacagttctggatacacaaaaacgtgtggggcgccaccgtgtagaacgcgcgccgattttaacgcatcaacggacctgctttgtcctacagattgctttcgcaaacatcctgataccacgtacttgaagtgtgggtctggaccctggctcaccccgcgatgtctcatcgactatccctaccggttgtggcactacccatgtaccgtcaactacaccatcttcaagattcggatgtacgtagggggtgttgagcaccgcctcaccgctgcctgcaatttcactaggggggaccgatgtaatcttgaggatcgcgatagaagtcaatagcttggtttctggagacaggctaaggatgcccttcaggtaccccgaggtaacaagcgacactcgggatctgagaaggggattggggcttctatccaagcgcccaatttaaaaagcttctatgcctgaataggtgaccggaggccggcaccttttcctttacccacaacttactttatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 118 - RNA, 2409 nt, synthetic construct
  source 1..2409: mol_type = other RNA
atggacgctatgaaacgagggctgtgctgcgtactgcttctttgtggtgcggtctttgtttctccgagcgcggaggttaaaaatatctcaaccggctacatggtgacgaacgactgtacgaacgactccattacatggcagcttcaggctgcggttctccatgtgccggggtgcgtgccttgcgaaaaagtcgggaatgcctcacaatgctggattccagtttcacccaacgtagcagtgcaacgaccaggtgcactcactcaaggtctgagaacccacattgatatggttgtaatgtctgcaacactctgttccgcactttatgtaggcgatctttgtggtggggtcatgcttgcagcccagatgtttattgtgagccctcagcaccattggttcgtacaggattgcaattgttctatctatcccggtacgattaccggacaccgaatggcgtgggacatgatgatgaactggtcacctaccgcgactatgattctggcctacgctatgcgcgttccagaggttatcattgacataatatctggtgcccattggggggtgatgtttggccttgcgtatttcagtatgcagggggcctgggccaaagtggttgttatcctgcttctcgccgcgggagtggatgcaaggactcatactgtaggcggctcagccgctcaaacgacagggcgattgactagtctctttgatatggggcctcgccaaaagatacaactggttaataccaacgggagttggcacatcaacagaacagcgctgaattgcaatgactccttgcacacaggtttcatcgctagtctcttttacacgcacagctttaatagctcaggttgtcccgaacggatgagtgcatgcagatcaattgaggcatttcgggtgggctggggtgcgctgcagtatgaggacaatgttacgaaccccgaggatatgcgaccctactgttggcattatcctccgagacagtgcggggttgtaagtgcgaaaactgtgtgtggtccagtgtattgctttacaccgagtcccgtcgttgttggtactaccgacaggttgggcgctcctacctatacctggggcgaaaatgagaccgatgtttttctgttgaacagtacacgacctccactgggcagttggttcggttgcacttggatgaacagttctggatacacaaaaacgtgtggggcgccaccgtgtagaacgcgcgccgattttaacgcatcaacggacctgctttgtcctacagattgctttcgcaaacatcctgataccacgtacttgaagtgtgggtctggaccctggctcaccccgcgatgtctcatcgactatccctaccggttgtggcactacccatgtaccgtcaactacaccatcttcaagattcggatgtacgtagggggtgttgagcaccgcctcaccgctgcctgcaatttcactaggggggaccgatgtaatcttgaggatcgcgatagaagtcaattgagcccgctcttgcactcaactaccgagtgggctattttgccatgcagctattcagacttgccagccttgagcaccggcctcctgcatctgcatcaaaatatcgtcgacgtacagttcatgtatggactgtctcctgcactgactaaatatatcgtccgctgggagtgggtgatcctcctcttcttgctccttgctgatgcgagggtctgtgcctgcttgtggatgctgatcctgcttggacaagcggaagcctagcttggtttctggagacaggctaaggatgcccttcaggtaccccgaggtaacaagcgacactcgggatctgagaaggggattggggcttctatccaagcgcccaatttaaaaagcttctatgcctgaataggtgaccggaggccggcaccttttcctttacccacaacttactttatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 119 - RNA, 1439 nt, synthetic construct
  source 1..1439: mol_type = other RNA
atggacgctatgaaacgagggctgtgctgcgtactgcttctttgtggtgcggtctttgtttctccgagcttggaatggagaaacacgagtggtctttatgtcttgaccaatgattgttccaactctagcatcgtgtatgaggctgatgatgtgatcctccacacgcccgggtgtgtgccgtgcgtccaagacgataatacttccacttgctggactcccgtaactcctactgttgctgttcgatatgtcggtgcaacgacagcgtctattcggagccacgtggacctgttggttggagcagctactctttgcagcgctctttatgtcggggatatgtgcggagcggttttccttgttggtcaggcgtttacatttcgaccccgacgacatcaaacggtgcaaacatgcaattgcagtctctacccgggacatgtatctgggcacaggatggcctgggacatgatgatgaattggtcccccgccgtagggatggtagttgcgcatatactccgacttccccagacgctgttcgacattttggctggcgcacattggggtatacttgccggtttggcttattatagcatgcagggaaactagttcgataccaagagctggtgtctgagggattacgactcgcgtggtcggacagcacatgttagtcatgtattatggtgtttcccaacacgtatctttgctctagtattgaactttagtgatgagtgtacttggcatgtacacctgattagttagcccttgtggatacgccactgccgctcaggtacagtaggacataccccaaatattgggttccactcgtagagaagaaaattagtggcgaaacgtacggtttatgtcaaaggagaaggtgattttaattgcacctgaagcaaccctaggtcttccctttgatgtgagctaataaggagtaaccgattcttacaatgtgatcaatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 120 - RNA, 1769 nt, synthetic construct
  source 1..1769: mol_type = other RNA
atggacgctatgaaacgagggctgtgctgcgtactgcttctttgtggtgcggtctttgtttctccgagcgagacatacgtcacggggggctcagtagcgcatagcgcccgggggcttaccagtctgttttcaatgggggcgaaacagaagctgcaacttgtaaacaccaacggtagttggcacatcaattctacggccctgaattgtaacgagagtattaacacgggctttatcgcggggctgttttattaccataagttcaattctaccggctgcccgcaacgactttcttcctgtaagccaatcattagtttccggcagggttggggccctctgaccgacgccaacattaccggtccgagcgacgaccgaccttattgttggcactatgcacctaggccgtgtagcgtggttcctgctagtagcgtatgcgggcccgtctattgttttacaccttctccggtagttgttgggactacagacatcaaaggcaaaccgacgtacaactggggcgaaaatgagacggatgtcttcctgctggagtccctgcggccacccagtggacgatggttcggatgcgcctggatgaactcaactggtttcctgaagacttgtggggctcccccgtgcaacatttatggaggtgagggcgacccagagaatgagacagacttgttttgtccgacagactgttttcggaagcatcccgaggctacgtacagtcgctgcggtgccggtccgtggctgacgccaagatgcatggtggattatccgtacagactttggcattatccttgtacggtcaactttactttgtttaaagttagaatgttcgtgggggggtttgaacatcggttcactgctgcctgtaactggacacgcggcgagcgatgtaacattgaagacagggacagaagtgaacagcattagttcgataccaagagctggtgtctgagggattacgactcgcgtggtcggacagcacatgttagtcatgtattatggtgtttcccaacacgtatctttgctctagtattgaactttagtgatgagtgtacttggcatgtacacctgattagttagcccttgtggatacgccactgccgctcaggtacagtaggacataccccaaatattgggttccactcgtagagaagaaaattagtggcgaaacgtacggtttatgtcaaaggagaaggtgattttaattgcacctgaagcaaccctaggtcttccctttgatgtgagctaataaggagtaaccgattcttacaatgtgatcaatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 121 - RNA, 2594 nt, synthetic construct
  source 1..2594: mol_type = other RNA
atggacgctatgaaacgagggctgtgctgcgtactgcttctttgtggtgcggtctttgtttctccgagcttggaatggagaaacacgagtggtctttatgtcttgaccaatgattgttccaactctagcatcgtgtatgaggctgatgatgtgatcctccacacgcccgggtgtgtgccgtgcgtccaagacgataatacttccacttgctggactcccgtaactcctactgttgctgttcgatatgtcggtgcaacgacagcgtctattcggagccacgtggacctgttggttggagcagctactctttgcagcgctctttatgtcggggatatgtgcggagcggttttccttgttggtcaggcgtttacatttcgaccccgacgacatcaaacggtgcaaacatgcaattgcagtctctacccgggacatgtatctgggcacaggatggcctgggacatgatgatgaattggtcccccgccgtagggatggtagttgcgcatatactccgacttccccagacgctgttcgacattttggctggcgcacattggggtatacttgccggtttggcttattatagcatgcagggaaactgggcaaaagtggcgattgtgatgattatgttcagcggggtggatgcagagacatacgtcacggggggctcagtagcgcatagcgcccgggggcttaccagtctgttttcaatgggggcgaaacagaagctgcaacttgtaaacaccaacggtagttggcacatcaattctacggccctgaattgtaacgagagtattaacacgggctttatcgcggggctgttttattaccataagttcaattctaccggctgcccgcaacgactttcttcctgtaagccaatcattagtttccggcagggttggggccctctgaccgacgccaacattaccggtccgagcgacgaccgaccttattgttggcactatgcacctaggccgtgtagcgtggttcctgctagtagcgtatgcgggcccgtctattgttttacaccttctccggtagttgttgggactacagacatcaaaggcaaaccgacgtacaactggggcgaaaatgagacggatgtcttcctgctggagtccctgcggccacccagtggacgatggttcggatgcgcctggatgaactcaactggtttcctgaagacttgtggggctcccccgtgcaacatttatggaggtgagggcgacccagagaatgagacagacttgttttgtccgacagactgttttcggaagcatcccgaggctacgtacagtcgctgcggtgccggtccgtggctgacgccaagatgcatggtggattatccgtacagactttggcattatccttgtacggtcaactttactttgtttaaagttagaatgttcgtgggggggtttgaacatcggttcactgctgcctgtaactggacacgcggcgagcgatgtaacattgaagacagggacagaagtgaacagcatccgctgctccattctactaccgaactcgctatactgccatgctcattcacacctatgccagctctctccaccgggttgatccacctccaccaaaatatcgtagacgttcaatacctttacggtgttgggtccgacatggtcggctgggcgttgaaatgggaattcgttatactcgtctttctcttgctcgcggatgcccgagtttgcgtagcactttggcttatgcttatggtatcacaggccgaggcatagttcgataccaagagctggtgtctgagggattacgactcgcgtggtcggacagcacatgttagtcatgtattatggtgtttcccaacacgtatctttgctctagtattgaactttagtgatgagtgtacttggcatgtacacctgattagttagcccttgtggatacgccactgccgctcaggtacagtaggacataccccaaatattgggttccactcgtagagaagaaaattagtggcgaaacgtacggtttatgtcaaaggagaaggtgattttaattgcacctgaagcaaccctaggtcttccctttgatgtgagctaataaggagtaaccgattcttacaatgtgatcaatgggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 122 - RNA, 2718 nt, synthetic construct
  source 1..2718: mol_type = other RNA
atgtcaacaaatcctaaaccccagcgaaagacaaagagaaataccaacagacggccccaggatgtgaaattcccgggtggcggacagatcgttggcggagtctacctgcttccacggcgagggcccagactgggtgttagagcgacccgcaaaacgtcagagaggagccaaccccgcgggcggcgccagccgatacccaaagcaaggagacctgagggtagaacttgggcccaacccggctatccttggccactgtatggaaacgagggatgcgggtgggcgggatggctgctgtccccgcgcggatctagaccttcttgggggccaacggatccgagacggcgaagccggaatttgggcaaagttattgacactcttacttgcgggtttgctgatttgatgggatacatacctctcgtaggtgcgccactcggaggggctgctcgagctctcgctcatggcgtgagagtactcgaggacggtgtaaattatgctaccggcaatctgccaggctgctccttttccatattcctgttggccctcctgagctgcttgaccgtccctgcttccgcctaccaagtacgaaacagtagtggcctctaccatgtcacaaacgactgcccaaatagcagtatagtatacgaagcggcagacgccatccttcatactccgggatgtgtcccttgtgtgcgagagggtaacgcatcacgatgttgggtcgcggttacacccacagtcgcaacgcgagacggaaagttgccgacaactcagttgcgaaggcatatagatttgctggttggctctgctaccctttgtagcgcactctacgtcggagacctgtgcggatctgtatttctcgttggtcaactgtttacattcagcccaagacgacattggactacccaggactgtaactgctccatatatcctggccatatcactggccacaggatggcgtgggacatgatgatgaactggagtcccaccgccgccttggtcgttgcgcagctgttgcgcattccccaagctattatggatatgattgcgggagcgcactggggagtcctggcaggtatcgcttatttcagtatggtggggaactgggcaaaggtgttggtggtcttgttgttgtttgctggggttgacgccgaaacccacgttacgggcggtaacgccggaaggacgacggcgggtctcgtcggattgttgacgcccggagcgaaacaaaatattcaattgataaataccaatggctcctggcatattaactcaactgcgctcaattgtaatgaaagtctcaacaccggttggctcgcaggtctcttctaccagcataaatttaattcatctggctgtccagaacggcttgctagttgcaggcgactgaccgacttcgcacagggatggggtccaatttcttacgcaaatggaagtggactggacgaaagaccgtattgctggcattatcctcctcgcccctgcggtatcgtgccggccaaaagcgtctgcggtcccgtctactgctttactccttctccagtagtggtcggtaccacggacaggtctggggcccctacgtactcctggggagcaaatgatacagacgtattcgtccttaacaatacgcgacccccgcttggcaactggtttggatgcacctggatgaattcaaccggattcaccaaggtatgtggtgcgccaccttgtgtcatagggggcgttggaaacaacactctgctctgtcccaccgattgttttcgcaaacatcctgaggctacgtatagtcgctgcggaagcggcccttggattactccacgatgtatggttgactacccctatagactctggcattacccatgcaccataaattatacaatttttaaggttcggatgtacgtcggtggcgtggaacacagactggaagctgcttgcaattggactcgaggcgaacgatgcgatcttgaggatcgagataggagcgagctctctcccctgctgcttagtacgacacaatggcaagttttgccgtgctcctttactaccttgcccgcgttgagcactggacttattcacttgcaccagaatatcgtagacgtccaatatttgtacggtgtcggatccagcattgccagctgggctattaagtgggagtatgtcgtactcctctttctgctgcttgccgatgcaagagtatgcagttgtttgtggatgatgctcctgatatcacaggcagaaggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 123 - RNA, 2733 nt, synthetic construct
  source 1..2733: mol_type = other RNA
atgagtacaaatccaaaaccccaaaggaagacaaaacgaaatacaaataggcgccctcaagatgttaagtttccgggaggtggccagatcgtcggtggggtgtatctcctcccacggagagggccgagactgggcgtgcgcgcgacgcgcaagacatctgaacggtcacaaccacgaggcagacgacagcccatacctaaggatcgacgatccacgggcaagtcctgggggaaaccggggtatccctggccgctttacggaaacgaaggcttggggtgggctggctggctcctgtctccaagaggcagccgaccttcttggggccccaatgacccgcgacaccggagtcggaatgtggggaaggtaatagatacattgacctgtggatttgctgacctcatggggtatatcccagtcgtgggcgcccccctgggtggtgtagcacgagccctggcgcatggggttagagtgctggaggatggagtgaatttcgccacgggaaatctccccggttgcagcttctcaatctttttgcttgcacttttgagctgcattactacaccagtttctgctgcggaggttaaaaatatctcaaccggctacatggtgacgaacgactgtacgaacgactccattacatggcagcttcaggctgcggttctccatgtgccggggtgcgtgccttgcgaaaaagtcgggaatgcctcacaatgctggattccagtttcacccaacgtagcagtgcaacgaccaggtgcactcactcaaggtctgagaacccacattgatatggttgtaatgtctgcaacactctgttccgcactttatgtaggcgatctttgtggtggggtcatgcttgcagcccagatgtttattgtgagccctcagcaccattggttcgtacaggattgcaattgttctatctatcccggtacgattaccggacaccgaatggcgtgggacatgatgatgaactggtcacctaccgcgactatgattctggcctacgctatgcgcgttccagaggttatcattgacataatatctggtgcccattggggggtgatgtttggccttgcgtatttcagtatgcagggggcctgggccaaagtggttgttatcctgcttctcgccgcgggagtggatgcaaggactcatactgtaggcggctcagccgctcaaacgacagggcgattgactagtctctttgatatggggcctcgccaaaagatacaactggttaataccaacgggagttggcacatcaacagaacagcgctgaattgcaatgactccttgcacacaggtttcatcgctagtctcttttacacgcacagctttaatagctcaggttgtcccgaacggatgagtgcatgcagatcaattgaggcatttcgggtgggctggggtgcgctgcagtatgaggacaatgttacgaaccccgaggatatgcgaccctactgttggcattatcctccgagacagtgcggggttgtaagtgcgaaaactgtgtgtggtccagtgtattgctttacaccgagtcccgtcgttgttggtactaccgacaggttgggcgctcctacctatacctggggcgaaaatgagaccgatgtttttctgttgaacagtacacgacctccactgggcagttggttcggttgcacttggatgaacagttctggatacacaaaaacgtgtggggcgccaccgtgtagaacgcgcgccgattttaacgcatcaacggacctgctttgtcctacagattgctttcgcaaacatcctgataccacgtacttgaagtgtgggtctggaccctggctcaccccgcgatgtctcatcgactatccctaccggttgtggcactacccatgtaccgtcaactacaccatcttcaagattcggatgtacgtagggggtgttgagcaccgcctcaccgctgcctgcaatttcactaggggggaccgatgtaatcttgaggatcgcgatagaagtcaattgagcccgctcttgcactcaactaccgagtgggctattttgccatgcagctattcagacttgccagccttgagcaccggcctcctgcatctgcatcaaaatatcgtcgacgtacagttcatgtatggactgtctcctgcactgactaaatatatcgtccgctgggagtgggtgatcctcctcttcttgctccttgctgatgcgagggtctgtgcctgcttgtggatgctgatcctgcttggacaagcggaagccggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 124 - RNA, 2739 nt, synthetic construct
  source 1..2739: mol_type = other RNA
atgagtactcttccaaagccccagagaaaaacgaagaggaatacaatcagacgcccacaggacgtgaagttcccagggggaggacagatcgttggtggagtatacgtacttccaagaagagggcctcgccttggcgttcgggctacccgaaagacgagtgagaggagccaacctcggggtcggcgccagccgatccccaaggctcggcggtccgaggggcggtcatgggcacagccgggttacccctggcctctttatggtaacgagggatgcggatgggctgggtggctcttgtctccccggggcagtcggccaagttggggaccaaatgatccccggcggcgctctcggaatctggggaaggtaatagacacacttacctgcggttttgcagatcttatgggatacatcccacttgtaggcgctccagtagggggtgtggcgcgcgccctcgcacatggtgtgcgggccttggaggatggtatcaattttgcgacgggcaatctgccaggatgttcattctccattttccttctcgctcttttctcttgtctcgtacaccccgcggcgagtttggaatggagaaacacgagtggtctttatgtcttgaccaatgattgttccaactctagcatcgtgtatgaggctgatgatgtgatcctccacacgcccgggtgtgtgccgtgcgtccaagacgataatacttccacttgctggactcccgtaactcctactgttgctgttcgatatgtcggtgcaacgacagcgtctattcggagccacgtggacctgttggttggagcagctactctttgcagcgctctttatgtcggggatatgtgcggagcggttttccttgttggtcaggcgtttacatttcgaccccgacgacatcaaacggtgcaaacatgcaattgcagtctctacccgggacatgtatctgggcacaggatggcctgggacatgatgatgaattggtcccccgccgtagggatggtagttgcgcatatactccgacttccccagacgctgttcgacattttggctggcgcacattggggtatacttgccggtttggcttattatagcatgcagggaaactgggcaaaagtggcgattgtgatgattatgttcagcggggtggatgcagagacatacgtcacggggggctcagtagcgcatagcgcccgggggcttaccagtctgttttcaatgggggcgaaacagaagctgcaacttgtaaacaccaacggtagttggcacatcaattctacggccctgaattgtaacgagagtattaacacgggctttatcgcggggctgttttattaccataagttcaattctaccggctgcccgcaacgactttcttcctgtaagccaatcattagtttccggcagggttggggccctctgaccgacgccaacattaccggtccgagcgacgaccgaccttattgttggcactatgcacctaggccgtgtagcgtggttcctgctagtagcgtatgcgggcccgtctattgttttacaccttctccggtagttgttgggactacagacatcaaaggcaaaccgacgtacaactggggcgaaaatgagacggatgtcttcctgctggagtccctgcggccacccagtggacgatggttcggatgcgcctggatgaactcaactggtttcctgaagacttgtggggctcccccgtgcaacatttatggaggtgagggcgacccagagaatgagacagacttgttttgtccgacagactgttttcggaagcatcccgaggctacgtacagtcgctgcggtgccggtccgtggctgacgccaagatgcatggtggattatccgtacagactttggcattatccttgtacggtcaactttactttgtttaaagttagaatgttcgtgggggggtttgaacatcggttcactgctgcctgtaactggacacgcggcgagcgatgtaacattgaagacagggacagaagtgaacagcatccgctgctccattctactaccgaactcgctatactgccatgctcattcacacctatgccagctctctccaccgggttgatccacctccaccaaaatatcgtagacgttcaatacctttacggtgttgggtccgacatggtcggctgggcgttgaaatgggaattcgttatactcgtctttctcttgctcgcggatgcccgagtttgcgtagcactttggcttatgcttatggtatcacaggccgaggcaggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 125 - RNA, 1080 nt, synthetic construct
  source 1..1080: mol_type = other RNA
atggacgctatgaaacgagggctgtgctgcgtactgcttctttgtggtgcggtctttgtttctccgagctaccaagtacgaaacagtagtggcctctaccatgtcacaaacgactgcccaaatagcagtatagtatacgaagcggcagacgccatccttcatactccgggatgtgtcccttgtgtgcgagagggtaacgcatcacgatgttgggtcgcggttacacccacagtcgcaacgcgagacggaaagttgccgacaactcagttgcgaaggcatatagatttgctggttggctctgctaccctttgtagcgcactctacgtcggagacctgtgcggatctgtatttctcgttggtcaactgtttacattcagcccaagacgacattggactacccaggactgtaactgctccatatatcctggccatatcactggccacaggatggcgtgggacatgatgatgaactggagtcccaccgccgccttggtcgttgcgcagctgttgcgcattccccaagctattatggatatgattgcgggagcgcactggggagtcctggcaggtatcgcttatttcagtatggtggggaacggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 126 - RNA, 1386 nt, synthetic construct
  source 1..1386: mol_type = other RNA
atggacgctatgaaacgagggctgtgctgcgtactgcttctttgtggtgcggtctttgtttctccgagcgaaacccacgttacgggcggtaacgccggaaggacgacggcgggtctcgtcggattgttgacgcccggagcgaaacaaaatattcaattgataaataccaatggctcctggcatattaactcaactgcgctcaattgtaatgaaagtctcaacaccggttggctcgcaggtctcttctaccagcataaatttaattcatctggctgtccagaacggcttgctagttgcaggcgactgaccgacttcgcacagggatggggtccaatttcttacgcaaatggaagtggactggacgaaagaccgtattgctggcattatcctcctcgcccctgcggtatcgtgccggccaaaagcgtctgcggtcccgtctactgctttactccttctccagtagtggtcggtaccacggacaggtctggggcccctacgtactcctggggagcaaatgatacagacgtattcgtccttaacaatacgcgacccccgcttggcaactggtttggatgcacctggatgaattcaaccggattcaccaaggtatgtggtgcgccaccttgtgtcatagggggcgttggaaacaacactctgctctgtcccaccgattgttttcgcaaacatcctgaggctacgtatagtcgctgcggaagcggcccttggattactccacgatgtatggttgactacccctatagactctggcattacccatgcaccataaattatacaatttttaaggttcggatgtacgtcggtggcgtggaacacagactggaagctgcttgcaattggactcgaggcgaacgatgcgatcttgaggatcgagataggagcgagggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 127 - RNA, 2214 nt, synthetic construct
  source 1..2214: mol_type = other RNA
atggacgctatgaaacgagggctgtgctgcgtactgcttctttgtggtgcggtctttgtttctccgagctaccaagtacgaaacagtagtggcctctaccatgtcacaaacgactgcccaaatagcagtatagtatacgaagcggcagacgccatccttcatactccgggatgtgtcccttgtgtgcgagagggtaacgcatcacgatgttgggtcgcggttacacccacagtcgcaacgcgagacggaaagttgccgacaactcagttgcgaaggcatatagatttgctggttggctctgctaccctttgtagcgcactctacgtcggagacctgtgcggatctgtatttctcgttggtcaactgtttacattcagcccaagacgacattggactacccaggactgtaactgctccatatatcctggccatatcactggccacaggatggcgtgggacatgatgatgaactggagtcccaccgccgccttggtcgttgcgcagctgttgcgcattccccaagctattatggatatgattgcgggagcgcactggggagtcctggcaggtatcgcttatttcagtatggtggggaactgggcaaaggtgttggtggtcttgttgttgtttgctggggttgacgccgaaacccacgttacgggcggtaacgccggaaggacgacggcgggtctcgtcggattgttgacgcccggagcgaaacaaaatattcaattgataaataccaatggctcctggcatattaactcaactgcgctcaattgtaatgaaagtctcaacaccggttggctcgcaggtctcttctaccagcataaatttaattcatctggctgtccagaacggcttgctagttgcaggcgactgaccgacttcgcacagggatggggtccaatttcttacgcaaatggaagtggactggacgaaagaccgtattgctggcattatcctcctcgcccctgcggtatcgtgccggccaaaagcgtctgcggtcccgtctactgctttactccttctccagtagtggtcggtaccacggacaggtctggggcccctacgtactcctggggagcaaatgatacagacgtattcgtccttaacaatacgcgacccccgcttggcaactggtttggatgcacctggatgaattcaaccggattcaccaaggtatgtggtgcgccaccttgtgtcatagggggcgttggaaacaacactctgctctgtcccaccgattgttttcgcaaacatcctgaggctacgtatagtcgctgcggaagcggcccttggattactccacgatgtatggttgactacccctatagactctggcattacccatgcaccataaattatacaatttttaaggttcggatgtacgtcggtggcgtggaacacagactggaagctgcttgcaattggactcgaggcgaacgatgcgatcttgaggatcgagataggagcgagctctctcccctgctgcttagtacgacacaatggcaagttttgccgtgctcctttactaccttgcccgcgttgagcactggacttattcacttgcaccagaatatcgtagacgtccaatatttgtacggtgtcggatccagcattgccagctgggctattaagtgggagtatgtcgtactcctctttctgctgcttgccgatgcaagagtatgcagttgtttgtggatgatgctcctgatatcacaggcagaaggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 128 - RNA, 1080 nt, synthetic construct
  source 1..1080: mol_type = other RNA
atggacgctatgaaacgagggctgtgctgcgtactgcttctttgtggtgcggtctttgtttctccgagcgcggaggttaaaaatatctcaaccggctacatggtgacgaacgactgtacgaacgactccattacatggcagcttcaggctgcggttctccatgtgccggggtgcgtgccttgcgaaaaagtcgggaatgcctcacaatgctggattccagtttcacccaacgtagcagtgcaacgaccaggtgcactcactcaaggtctgagaacccacattgatatggttgtaatgtctgcaacactctgttccgcactttatgtaggcgatctttgtggtggggtcatgcttgcagcccagatgtttattgtgagccctcagcaccattggttcgtacaggattgcaattgttctatctatcccggtacgattaccggacaccgaatggcgtgggacatgatgatgaactggtcacctaccgcgactatgattctggcctacgctatgcgcgttccagaggttatcattgacataatatctggtgcccattggggggtgatgtttggccttgcgtatttcagtatgcagggggccggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 129 - RNA, 1398 nt, synthetic construct
  source 1..1398: mol_type = other RNA
atggacgctatgaaacgagggctgtgctgcgtactgcttctttgtggtgcggtctttgtttctccgagcaggactcatactgtaggcggctcagccgctcaaacgacagggcgattgactagtctctttgatatggggcctcgccaaaagatacaactggttaataccaacgggagttggcacatcaacagaacagcgctgaattgcaatgactccttgcacacaggtttcatcgctagtctcttttacacgcacagctttaatagctcaggttgtcccgaacggatgagtgcatgcagatcaattgaggcatttcgggtgggctggggtgcgctgcagtatgaggacaatgttacgaaccccgaggatatgcgaccctactgttggcattatcctccgagacagtgcggggttgtaagtgcgaaaactgtgtgtggtccagtgtattgctttacaccgagtcccgtcgttgttggtactaccgacaggttgggcgctcctacctatacctggggcgaaaatgagaccgatgtttttctgttgaacagtacacgacctccactgggcagttggttcggttgcacttggatgaacagttctggatacacaaaaacgtgtggggcgccaccgtgtagaacgcgcgccgattttaacgcatcaacggacctgctttgtcctacagattgctttcgcaaacatcctgataccacgtacttgaagtgtgggtctggaccctggctcaccccgcgatgtctcatcgactatccctaccggttgtggcactacccatgtaccgtcaactacaccatcttcaagattcggatgtacgtagggggtgttgagcaccgcctcaccgctgcctgcaatttcactaggggggaccgatgtaatcttgaggatcgcgatagaagtcaaggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 130 - RNA, 2229 nt, synthetic construct
  source 1..2229: mol_type = other RNA
atggacgctatgaaacgagggctgtgctgcgtactgcttctttgtggtgcggtctttgtttctccgagcgcggaggttaaaaatatctcaaccggctacatggtgacgaacgactgtacgaacgactccattacatggcagcttcaggctgcggttctccatgtgccggggtgcgtgccttgcgaaaaagtcgggaatgcctcacaatgctggattccagtttcacccaacgtagcagtgcaacgaccaggtgcactcactcaaggtctgagaacccacattgatatggttgtaatgtctgcaacactctgttccgcactttatgtaggcgatctttgtggtggggtcatgcttgcagcccagatgtttattgtgagccctcagcaccattggttcgtacaggattgcaattgttctatctatcccggtacgattaccggacaccgaatggcgtgggacatgatgatgaactggtcacctaccgcgactatgattctggcctacgctatgcgcgttccagaggttatcattgacataatatctggtgcccattggggggtgatgtttggccttgcgtatttcagtatgcagggggcctgggccaaagtggttgttatcctgcttctcgccgcgggagtggatgcaaggactcatactgtaggcggctcagccgctcaaacgacagggcgattgactagtctctttgatatggggcctcgccaaaagatacaactggttaataccaacgggagttggcacatcaacagaacagcgctgaattgcaatgactccttgcacacaggtttcatcgctagtctcttttacacgcacagctttaatagctcaggttgtcccgaacggatgagtgcatgcagatcaattgaggcatttcgggtgggctggggtgcgctgcagtatgaggacaatgttacgaaccccgaggatatgcgaccctactgttggcattatcctccgagacagtgcggggttgtaagtgcgaaaactgtgtgtggtccagtgtattgctttacaccgagtcccgtcgttgttggtactaccgacaggttgggcgctcctacctatacctggggcgaaaatgagaccgatgtttttctgttgaacagtacacgacctccactgggcagttggttcggttgcacttggatgaacagttctggatacacaaaaacgtgtggggcgccaccgtgtagaacgcgcgccgattttaacgcatcaacggacctgctttgtcctacagattgctttcgcaaacatcctgataccacgtacttgaagtgtgggtctggaccctggctcaccccgcgatgtctcatcgactatccctaccggttgtggcactacccatgtaccgtcaactacaccatcttcaagattcggatgtacgtagggggtgttgagcaccgcctcaccgctgcctgcaatttcactaggggggaccgatgtaatcttgaggatcgcgatagaagtcaattgagcccgctcttgcactcaactaccgagtgggctattttgccatgcagctattcagacttgccagccttgagcaccggcctcctgcatctgcatcaaaatatcgtcgacgtacagttcatgtatggactgtctcctgcactgactaaatatatcgtccgctgggagtgggtgatcctcctcttcttgctccttgctgatgcgagggtctgtgcctgcttgtggatgctgatcctgcttggacaagcggaagccggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 131 - RNA, 1080 nt, synthetic construct
  source 1..1080: mol_type = other RNA
atggacgctatgaaacgagggctgtgctgcgtactgcttctttgtggtgcggtctttgtttctccgagcttggaatggagaaacacgagtggtctttatgtcttgaccaatgattgttccaactctagcatcgtgtatgaggctgatgatgtgatcctccacacgcccgggtgtgtgccgtgcgtccaagacgataatacttccacttgctggactcccgtaactcctactgttgctgttcgatatgtcggtgcaacgacagcgtctattcggagccacgtggacctgttggttggagcagctactctttgcagcgctctttatgtcggggatatgtgcggagcggttttccttgttggtcaggcgtttacatttcgaccccgacgacatcaaacggtgcaaacatgcaattgcagtctctacccgggacatgtatctgggcacaggatggcctgggacatgatgatgaattggtcccccgccgtagggatggtagttgcgcatatactccgacttccccagacgctgttcgacattttggctggcgcacattggggtatacttgccggtttggcttattatagcatgcagggaaacggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 132 - RNA, 1410 nt, synthetic construct
  source 1..1410: mol_type = other RNA
atggacgctatgaaacgagggctgtgctgcgtactgcttctttgtggtgcggtctttgtttctccgagcgagacatacgtcacggggggctcagtagcgcatagcgcccgggggcttaccagtctgttttcaatgggggcgaaacagaagctgcaacttgtaaacaccaacggtagttggcacatcaattctacggccctgaattgtaacgagagtattaacacgggctttatcgcggggctgttttattaccataagttcaattctaccggctgcccgcaacgactttcttcctgtaagccaatcattagtttccggcagggttggggccctctgaccgacgccaacattaccggtccgagcgacgaccgaccttattgttggcactatgcacctaggccgtgtagcgtggttcctgctagtagcgtatgcgggcccgtctattgttttacaccttctccggtagttgttgggactacagacatcaaaggcaaaccgacgtacaactggggcgaaaatgagacggatgtcttcctgctggagtccctgcggccacccagtggacgatggttcggatgcgcctggatgaactcaactggtttcctgaagacttgtggggctcccccgtgcaacatttatggaggtgagggcgacccagagaatgagacagacttgttttgtccgacagactgttttcggaagcatcccgaggctacgtacagtcgctgcggtgccggtccgtggctgacgccaagatgcatggtggattatccgtacagactttggcattatccttgtacggtcaactttactttgtttaaagttagaatgttcgtgggggggtttgaacatcggttcactgctgcctgtaactggacacgcggcgagcgatgtaacattgaagacagggacagaagtgaacagcatggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 133 - RNA, 2235 nt, synthetic construct
  source 1..2235: mol_type = other RNA
atggacgctatgaaacgagggctgtgctgcgtactgcttctttgtggtgcggtctttgtttctccgagcttggaatggagaaacacgagtggtctttatgtcttgaccaatgattgttccaactctagcatcgtgtatgaggctgatgatgtgatcctccacacgcccgggtgtgtgccgtgcgtccaagacgataatacttccacttgctggactcccgtaactcctactgttgctgttcgatatgtcggtgcaacgacagcgtctattcggagccacgtggacctgttggttggagcagctactctttgcagcgctctttatgtcggggatatgtgcggagcggttttccttgttggtcaggcgtttacatttcgaccccgacgacatcaaacggtgcaaacatgcaattgcagtctctacccgggacatgtatctgggcacaggatggcctgggacatgatgatgaattggtcccccgccgtagggatggtagttgcgcatatactccgacttccccagacgctgttcgacattttggctggcgcacattggggtatacttgccggtttggcttattatagcatgcagggaaactgggcaaaagtggcgattgtgatgattatgttcagcggggtggatgcagagacatacgtcacggggggctcagtagcgcatagcgcccgggggcttaccagtctgttttcaatgggggcgaaacagaagctgcaacttgtaaacaccaacggtagttggcacatcaattctacggccctgaattgtaacgagagtattaacacgggctttatcgcggggctgttttattaccataagttcaattctaccggctgcccgcaacgactttcttcctgtaagccaatcattagtttccggcagggttggggccctctgaccgacgccaacattaccggtccgagcgacgaccgaccttattgttggcactatgcacctaggccgtgtagcgtggttcctgctagtagcgtatgcgggcccgtctattgttttacaccttctccggtagttgttgggactacagacatcaaaggcaaaccgacgtacaactggggcgaaaatgagacggatgtcttcctgctggagtccctgcggccacccagtggacgatggttcggatgcgcctggatgaactcaactggtttcctgaagacttgtggggctcccccgtgcaacatttatggaggtgagggcgacccagagaatgagacagacttgttttgtccgacagactgttttcggaagcatcccgaggctacgtacagtcgctgcggtgccggtccgtggctgacgccaagatgcatggtggattatccgtacagactttggcattatccttgtacggtcaactttactttgtttaaagttagaatgttcgtgggggggtttgaacatcggttcactgctgcctgtaactggacacgcggcgagcgatgtaacattgaagacagggacagaagtgaacagcatccgctgctccattctactaccgaactcgctatactgccatgctcattcacacctatgccagctctctccaccgggttgatccacctccaccaaaatatcgtagacgttcaatacctttacggtgttgggtccgacatggtcggctgggcgttgaaatgggaattcgttatactcgtctttctcttgctcgcggatgcccgagtttgcgtagcactttggcttatgcttatggtatcacaggccgaggcaggagtgtacctgctgcctcggcggggccctagactgggcgtgcgggctacaagaaagaccagcgaaagaagccagcctagaggcagacgccagccaatccccaaggccagacggtccgagggcagatcttgggcccagcctggctaccccaaatctaccaaggtgcctgccgcttacgccgcccagggctacaaggtcctggtgctgaaccccagcgtggccgctacactgggattcggcgcctacatgagcaagctggaacagatcaagggcggcagacacctgatcttctgtcacagcaagaaaaagtgcgacgagctggccgccaagctgcggatcctgagacggcacgtcggccctggagaaggcgccgtgcagtggatgaaccggctgatcgccttcgccagcagaggcaaccacgtgtctcctacacactacgtgcccgagagcgacgccgccgctagagtgacccagatcctgtag

SEQ ID NO: 134 - DNA, 2238 bp, Hepacivirus C
  source 1..2238: mol_type = genomic DNA
atgagtactaaccccaagccccaacggaagacaaaacggaacacaaaccgccggccgatggatgttaaatttccaggcggtggtcaaatagtgggtggggtttatcttcttccacgacgcgggcctcgactgggagtcagagcgacccgcaagacgagtgagagatctcagcccaggggtcgcaggcaaccaatcccaaaagcgcggcgcccagaagggagatcatgggcacaacctggatacccgtggcccctttacggtaacgagggctgtgggtgggcgggctggttgctcagtccaaggggctctcggcccagctggggtccaaatgatcctcgaggccgaagtcgcaatcttggtaaagttattgacacgctgacttgtggcttcgcggaccttatgggctatattcccctggttggtgccccagtaggctccgttgctagagctctcgcccacggtgtgcgggcgctggaagacgggattaattacgcgactgggaaccttcccggatgttctttcagcatttttttgctggcactcctgtcatgtctgactgtaccggcttctgccgtcaactacagaaacgtttctggtatttatcacgtcacgaatgattgtcctaattcatcaatcgtatatgaagctgatcatcacatcatgcatcttcctggctgcgtcccttgcgttcgagaggggaatcaaagtcgatgttgggtcgcgttgaccccaacggtagctgcaccttatattggggctccacttgagtcccttcgatcacacgtcgacctgatggtgggagctgcgacggtctgcagcggtctctacattggagatctgtgtggtggcctcttcctcgtaggccaaatgtttagttttagaccgcggcggcattggactactcaggattgtaactgctctatatacacggggcacattacgggccataggatggcgtgggacatgatgatgaactggtctccgacgacgactttggttcttgctcaagtgatgaggataccaaccactcttgtcgatttgttgagtggaggccattggggcgtcctggtcggagtcgcatattttagtatgcaggcgaactgggctaaagtcatactcgtgcttttcttgtttgcgggggtagatgcggagacgcatgtatctggggccgccgtaggccggtccaccgctggattggctaacctgttctccagtggatctaaacagaacttgcagctcataaatagtaatggatcttggcatatcaataggacagcattgaattgtaatgattccctcaacacaggttttttggcttctctgttctacacacataaattcaacagttccggatgtagtgagcggctggcttgctgtaagtctttggattcttacggacagggttggggtcctcttggcgtagctaatatatccgggtcaagcgacgatcggccctattgctggcattacgctccacgaccgtgcggaatcgttcccgcctcatctgtatgtggtccagtatattgttttacaccgtcccccgtcgtggtaggaacaacagatcatgtgggcgtcccgacctacacgtggggggagaacgagacggatgttttcttgttgaactctacaagaccgcctcacggtgcatggtttgggtgcgtttggatgaactcaacaggctttacgaaaacctgcggtgcgccgccctgtgaggttaatactaacaacggaacctggcattgtcccacagactgcttcagaaagcacccggagacgacttatgccaagtgtggaagcggtccatggattacgcctcgatgtttgattgattatccttaccggctctggcactttccgtgcacggctaatttctccgttttcaacattagaacttttgtgggcggaattgaacaccggatgcaggctgcctgcaactggacaagaggagaagtatgtggacttgagcaccgggaccgcgtagaactcagccctctgcttctgacaaccactgcatggcagatacttccatgctccttcaccaccctgcccgcactgtcaacaggcctgatacacctccatcaaaacatagtggatgtgcagtatctgtatggggtgggttcagcagtggtcagctgggctttgaaatgggagtacgtggttttggccttcctgttgctcgccgatgcacgagtctctgcttatctgtggatgatgtttatggtgtcacaagtagaagcc

SEQ ID NO: 135 - protein, 746 aa, Hepacivirus C
MSTNPKPQRKTKRNTNRRPMDVKFPGGGQIVGGVYLLPRRGPRLGVRATRKTSERSQPRGRRQPIPKARRPEGRSWAQPGYPWPLYGNEGCGWAGWLLSPRGSRPSWGPNDPRGRSRNLGKVIDTLTCGFADLMGYIPLVGAPVGSVARALAHGVRALEDGINYATGNLPGCSFSIFLLALLSCLTVPASAVNYRNVSGIYHVTNDCPNSSIVYEADHHIMHLPGCVPCVREGNQSRCWVALTPTVAAPYIGAPLESLRSHVDLMVGAATVCSGLYIGDLCGGLFLVGQMFSFRPRRHWTTQDCNCSIYTGHITGHRMAWDMMMNWSPTTTLVLAQVMRIPTTLVDLLSGGHWGVLVGVAYFSMQANWAKVILVLFLFAGVDAETHVSGAAVGRSTAGLANLFSSGSKQNLQLINSNGSWHINRTALNCNDSLNTGFLASLFYTHKFNSSGCSERLACCKSLDSYGQGWGPLGVANISGSSDDRPYCWHYAPRPCGIVPASSVCGPVYCFTPSPVVVGTTDHVGVPTYTWGENETDVFLLNSTRPPHGAWFGCVWMNSTGFTKTCGAPPCEVNTNNGTWHCPTDCFRKHPETTYAKCGSGPWITPRCLIDYPYRLWHFPCTANFSVFNIRTFVGGIEHRMQAACNWTRGEVCGLEHRDRVELSPLLLTTTAWQILPCSFTTLPALSTGLIHLHQNIVDVQYLYGVGSAVVSWALKWEYVVLAFLLLADARVSAYLWMMFMVSQVEA

SEQ ID NO: 136 - DNA, 2241 bp, Hepacivirus C
  source 1..2241: mol_type = genomic DNA
atgtccaccaatccaaagccccagcgaaaaactaaacgaaacacgaacaggaggccgcaagacgttaagttccctggaggtgggcagatcgtgggaggggtttatctccttccacgaaggggaccccgcctgggagtgcgcgcaacacggaagacgtctgaacgatcccaaccgaggggcaggaggcaaccgatacctaaggctcgacaaccaactggacgctcctggggtcagccaggctatccttggccgttgtacgcgaatgaaggcctcggttgggcaggttggctgctttcccccagaggaagtcgcccgaattggggtccaaacgacccaaggaggaaaagccgaaacctggggaaggtcattgatacattgacttgtggtttcgctgatcttatgggctacattccgcttgtaggtggccccgtcggtggtgttgcgagagcgctcgcgcatggtgtacgcgtactggaggacggcgttaactatgccacagggaatctgcctgggtgttccttctcaatcttcatccttgctcttttgtcttgtcttactgtgcccacttccgctgtcccatatcgcaacgcctctggagtctaccatgtaacgaacgactgtccgaacagttccatagtctatgaggcagaggaccttattctccacgccccgggttgcgtcccgtgcgttagacaaggtaatgtgagtcgctgttgggtccaaattaccccgacgttgagcgcacccagccttggtgcagtaacggcgccgcttcgacgagcggtagactaccttgctggtggtgcagctctttgttccgctttgtacgtgggcgatgcttgcggtgcagtgtttctggtcggtcaaatgttcacgtatagtcctagacgacataacgttgttcaggattgtaactgctcaatctacagtgggcatataactgggcacagaatggcttgggacatgatgatgaactggagtcctacgacagccctggtgatggcacagctgcttcgaatacctcaggttgttattgacataatcgctggagcccattggggagttttgttcgctgctgcttactacgctagtgcggcaaactgggcgaaagtagtgctcgtactctttctctttgcgggcgtagatgcctctacacggactgtagggggtagtgcagcacagggagcgcgagggcttgccagtctcttcactccaggtccccaacaaaatctccagttgatcaacaccaacggttcttggcatattaatcgaactgccctcaactgcaacgactctcttcaaaccgggtttgtcgcggggctcctgtactaccataaattcaatagtaccgggtgtccccaaaggatggctagctgcaggcctctggctgcttttgatcaaggttggggtacaatcagttacgcggcggtcagtggacccagcgatgacaagccgtactgctggcactatcccccaaggccatgcggaattgtacctgcacgaggagtttgcggtccagtatactgttttacgccatcaccggtcgtcgtaggaacaactgaccggaagggcaatccaacgtacagttggggcgaaaacgaaacagatattttcctgctcaacaacacacgaccgccaacgggaaactggtttggctgcacgtggatgaactctactggctttgttaagacatgcggagcccccccatgtaaccttggccccacaggcaataacagccttaaatgtcccaccgattgtttccgcaagcatccagatgctacctacactaaatgcgggtctgggccatggttgactccgagatgcctcgtgcactatccttataggctctggcactacccttgcacacttaattacacaattttcaaagtaaggatgtatattggaggccttgagcacagacttgaggtcgcctgtaattggacgcgaggcgagcgctgtgacctcgaggatcgagatcgggctgagctctctccgttgcttcatacaaccacacaatgggctattcttccctgttccttcacgccaaccccagcattgagcactggcctgattcaccttcaccaaaatatagttgacacgcaatacctctatggtctcagttcctcaattgtgagctgggccgtcaaatgggaatatattgtcctcgcgttcttgctcctggcagatgcaagaatttgtacatgtttgtggatcatgctcttggtatgtcaagccgaggct

SEQ ID NO: 137 - protein, 747 aa, Hepacivirus C
MSTNPKPQRKTKRNTNRRPQDVKFPGGGQIVGGVYLLPRRGPRLGVRATRKTSERSQPRGRRQPIPKARQPTGRSWGQPGYPWPLYANEGLGWAGWLLSPRGSRPNWGPNDPRRKSRNLGKVIDTLTCGFADLMGYIPLVGGPVGGVARALAHGVRVLEDGVNYATGNLPGCSFSIFILALLSCLTVPTSAVPYRNASGVYHVTNDCPNSSIVYEAEDLILHAPGCVPCVRQGNVSRCWVQITPTLSAPSLGAVTAPLRRAVDYLAGGAALCSALYVGDACGAVFLVGQMFTYSPRRHNVVQDCNCSIYSGHITGHRMAWDMMMNWSPTTALVMAQLLRIPQVVIDIIAGAHWGVLFAAAYYASAANWAKVVLVLFLFAGVDASTRTVGGSAAQGARGLASLFTPGPQQNLQLINTNGSWHINRTALNCNDSLQTGFVAGLLYYHKFNSTGCPQRMASCRPLAAFDQGWGTISYAAVSGPSDDKPYCWHYPPRPCGIVPARGVCGPVYCFTPSPVVVGTTDRKGNPTYSWGENETDIFLLNNTRPPTGNWFGCTWMNSTGFVKTCGAPPCNLGPTGNNSLKCPTDCFRKHPDATYTKCGSGPWLTPRCLVHYPYRLWHYPCTLNYTIFKVRMYIGGLEHRLEVACNWTRGERCDLEDRDRAELSPLLHTTTQWAILPCSFTPTPALSTGLIHLHQNIVDTQYLYGLSSSIVSWAVKWEYIVLAFLLLADARICTCLWIMLLVCQAEA

SEQ ID NO: 138 - DNA, 2256 bp, Hepacivirus C
  source 1..2256: mol_type = genomic DNA
atgtctacactgcctaaacctcagagaaaaacgaagcggaacactaacagacggccaatggatgtgaagtttcctgggggcggacagatcgttggtggtgtgtacctgttgccccgacgggggcctcggctgggagtccgagcaactcgaaagacctctgagcgcagccagcctcgcgggcggcgacagccgattcccaaggctagacagccacaagggagacattgggcacagccaggttatccatggcccctgtacggaaatgagggctgtggctgggctggttggctcttgtctcctagagggtcaaggcctcactggggaccaaatgacccccgacgccgaagcagaaatttgggtaaagttatcgacacgctcacatgcggctttgcagacttgatgggttacataccagtagttggcgcgccgttggggggagtcgcggctgcgttggcacatggggttcgggcaattgaggatggcatcaactatgccacagggaatcttccgggctgcagcttcagtatatttctcctcgctctgttgagttgcctcaccacccccgcatccgcactcacctatgggaactcttccgggctctatcatcttactaatgactgcccgaatagtagtatcgtcctggaagctgacgcgatgatactgcatctgccgggctgtctgccttgcgtaagggtcaataacaatcagagtatctgttggcatgctgtctcacctacactggccatacccaatgcaagtacgcccgctactggcttccgacgacacgtcgatttgttggcaggcgcagctgttgtatgctccagcctttacattggagatttgtgcggatccctgtttctcgcgggtcagctttttacgtttcagccacgacgccactggacggtacaagattgtaactgctcaatatacaccggccatgttaccggtcatagaatggcctgggatatgatgatgaattggtctcctactaccacactcgttctgagttctatattgcgagtcccggagatatgcgcgagcgtcattttcggtggtcattgggggatcttgctcgccgtagcctatttcggcatggcaggtaactggctcaaggtattggcagttctctttctcttcgcaggggtcgaggccactaccactattggtcaccaggtgggtcgcacgactgggggtcttgctagcctgttttccataggtcctaggcagaacctccagctcatcaacactaatggctcttggcacattaacaggacggctctgaattgtaacgatagtttgcaaactggatttataacaagtcttttctacgctaaaaacgtaaattccagcggttgccctgaaaggatggccgcatgcaagccactcgctgactttcggcaagggtggggtcagataacttataaggtaaacatatcaggtccatcagacgataggccttactgttggcactacgcgcctagaccctgcgacgtagtttccgcaaggacggtctgcggtccagtttattgcttcacaccctctccagtggtagttggtacaaccgataagctcgggatccctacttacaattggggagagaacgagacagatgtgtttatgcttgaaagcctgcgacccccgaccggaggctggttcggctgcacctggatgaatagtacggggttcaccaagacttgtggcgcacccccttgccaaatagttccaggcgattacaactcttccgctaacgagctcctctgcccgaccgactgcttcagaaaacatccagaagccacataccaaaggtgtggctcaggaccttggataacccctagatgcctcgtcgactatccgtatagactgtggcactatccttgcacggtgaattttactctccataaggttagaatgttcgttggtggcatcgaacaccgatttgacgcagcttgtaattggacaaggggcgaacgctgtgatctccatgaccgggatcggatcgaaatgagtcccctccttttcagtaccacccagctcgcgatcctcccctgttccttctcaacgatgcccgccctctcaaccggcctgatacatctgcatcaaaacatcgtagacgtccaatatttgtacggtgtgtctagttctgtgacatcttgggtagttaagtgggaatatatagttttggtgtttctggtcctcgcagacgcgagaatttgcacgtgcctctggctgatgcttcttataactaacgtagaggct

SEQ ID NO: 139 - protein, 752 aa, Hepacivirus C
MSTLPKPQRKTKRNTNRRPMDVKFPGGGQIVGGVYLLPRRGPRLGVRATRKTSERSQPRGRRQPIPKARQPQGRHWAQPGYPWPLYGNEGCGWAGWLLSPRGSRPHWGPNDPRRRSRNLGKVIDTLTCGFADLMGYIPVVGAPLGGVAAALAHGVRAIEDGINYATGNLPGCSFSIFLLALLSCLTTPASALTYGNSSGLYHLTNDCPNSSIVLEADAMILHLPGCLPCVRVNNNQSICWHAVSPTLAIPNASTPATGFRRHVDLLAGAAVVCSSLYIGDLCGSLFLAGQLFTFQPRRHWTVQDCNCSIYTGHVTGHRMAWDMMMNWSPTTTLVLSSILRVPEICASVIFGGHWGILLAVAYFGMAGNWLKVLAVLFLFAGVEATTTIGHQVGRTTGGLASLFSIGPRQNLQLINTNGSWHINRTALNCNDSLQTGFITSLFYAKNVNSSGCPERMAACKPLADFRQGWGQITYKVNISGPSDDRPYCWHYAPRPCDVVSARTVCGPVYCFTPSPVVVGTTDKLGIPTYNWGENETDVFMLESLRPPTGGWFGCTWMNSTGFTKTCGAPPCQIVPGDYNSSANELLCPTDCFRKHPEATYQRCGSGPWITPRCLVDYPYRLWHYPCTVNFTLHKVRMFVGGIEHRFDAACNWTRGERCDLHDRDRIEMSPLLFSTTQLAILPCSFSTMPALSTGLIHLHQNIVDVQYLYGVSSSVTSWVVKWEYIVLVFLVLADARICTCLWLMLLITNVEA

SEQ ID NO: 140 - protein, 403 aa, synthetic construct
  VARIANT 100: note = X is Thr, Val, or Gln
  VARIANT 148: note = X is Thr, Asn, Gln, His, or Ser
  VARIANT 162: note = X is Glu, Ser, or Ala
  VARIANT 163: note = X is Val, Thr, Tyr, Phe, or Leu
  VARIANT 208: note = X is Ser, Gln, or Thr
  VARIANT 307: note = X is Glu, Thr, or Val
  VARIANT 346: note = X is Ser, Ala, or Pro
SYQVGYLHAPTGSGKSTKVPAAYAAQGYKVLVLNPSVAATLGFGAYMSKAHGIDPNIRTGVRTVTTGAPITYSTYGKFLADGGCSGGAYDIIICDECHSXDATTILGIGTVLDQAETAGARLVVLATATPPGSVTVPHPNIEEVALGXEGEIPFYGKAIPLXXIKGGRHLIFCHSKKKCDELAAKLRGMGLNAVAYYRGLDVSVIPTXGDVVVVATDALMTGYTGDFDSVIDCNVAVTQTILRRHVGPGEGAVQWMNRLIAFASRGNHVSPTHYVPESDAAARVTQILTPIDTTIMAKNEVFCVDPXKGGRKPARLIVYPDLGVRVCEKMALYDVVQKLPQAVMGXSYGFQYSGVYLLPRRGPRLGVRATRKTSERSQPRGRRQPIPKARRSEGRSWAQPGYP

SEQ ID NO: 141 - DNA, 4929 bp, synthetic construct
  source 1..4929: mol_type = other DNA
gggaaataagagagaaaagaagagtaagaagaaatataagagccaccatggatgctatgaagaggggcctgtgctgcgtgctgcttctgtgtggcgctgtgtttgtgtcccctagctaccaagtgcggaacagcagcggcctgtaccacgtgaccaacgactgccccaatagcagcatcgtgtacgaagccgccgatgccatcctgcacacacctggatgtgtgccctgtgtcagagagggcaacgccagcagatgttgggttgccgtgacacctaccgtggccaccagagatggaaagctgcctaccacacagctgcggagacacatcgatctgctcgtgggatccgccacactgtgtagcgctctgtatgtgggcgatctgtgcggctccgtgtttctcgtgggccagctgttcacattcagccccagaaggcactggaccacacaggactgcaactgcagcatctaccccggccacatcaccggacatagaatggcctgggacatgatgatgaattggagccccacagccgctctggtggttgctcagctgctgagaatccctcaggccatcatggacatgatcgctggcgctcattggggagtgctggccggaatcgcctacttcagcatggtcggaaactgggccaaagtgctggtggtgctgctgctttttgctggcgtggacgccgagacacatgtgacaggtggaaatgccggcagaaccacagccggacttgtgggacttcttacccctggcgccaagcagaacatccagctgatcaacaccaacggcagctggcacatcaacagcacagccctgaactgcaacgagagcctgaataccggatggctggccggcctgttctaccagcacaagttcaattccagcggctgccccgagagactggcctcttgtagaaggctgaccgacttcgcccaaggctggggccctatctcttacgccaatggctctggcctggacgagaggccttactgctggcactatcctccaagaccttgcggcatcgtgcctgccaagtctgtgtgtggacccgtgtactgcttcaccccatctccagtggtcgtgggcaccaccgatagatctggcgccccaacatatagctggggagccaacgacaccgacgtgttcgtgctgaacaataccagacctcctctcggcaattggttcggctgtacctggatgaacagcaccggcttcaccaaagtgtgcggagcccctccttgtgtgattggcggcgtgggaaacaacaccctgctgtgccctaccgactgcttcagaaagcaccctgaggccacctacagcagatgcggatctggcccttggatcacccctagatgcatggtggactacccctacagactgtggcactacccttgcaccatcaactacaccatcttcaaagtgcggatgtacgtcggcggagtggaacacagactggaagccgcctgcaattggaccagaggcgagagatgcgacctggaagatcgggacagaagcgagctgtctcctctgctgctgagcaccacacagtggcaggtcctgccttgcagcttcaccacactgccagctctgtctaccggcctgatccatctgcaccagaacatcgtggacgtgcagtacctgtacggcgtgggcagctctattgccagctgggccatcaagtgggagtacgtggtcctcctgttcctgctgctggccgatgctagagtgtgcagctgcctgtggatgatgctgctgatctctcaggccgagtagtaactcgaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccgctagcatgagcttccaggtggcccatctgcatgcccctacaggctctggcaagagcacaaaagtgcctgccgcctatgccgctcagggctataaggtgctggtgctgaatccctctgtggccgccacacttggctttggcgcctacatgtctaaagcccacggcgtggaccccaacatcagaaccggcgtgcggaccattacaacaggcgcccctatcacctactctacctacggcaagttcctggccgatggcggatgttctggcggagcctacgacatcatcatctgcgacgagtgccacagcaccgacgccacatctatcctcggcatcggcacagtgctggaccaggctgaaacagctggtgccagactggtggtgctggccacagctacacctccaggatctgtgaccgtgcctcatcctaacatcgaggaagtggccctgtctacaaccggcgagatcccattctatggcaaggccattcctctggaagtgatcaaaggcggcagacacctgatcttttgccactccaagaagaagtgcgacgagctggccgccaaactggtggcccttggaatcaatgccgtggcctactacagaggcctggacgtgtccgtgattcctacatctggcgacgtggtggtggtcgccactgatgctctgatgaccggcttcaccggcgacttcgacagcgtgatcgactgcaatacctgcgtgacccagaccgtggacttctctctggaccccaccttcaccatcgagacaaccacactgcctcaggacgccgtgtctcggacacagagaagaggcagaacaggcagaggcaagcccggcatctacagatttgtggcccctggcgaaagacccagcggcatgtttgatagcagcgtgctgtgcgagtgctacgatgccggatgtgcttggtacgagctgacccctgccgaaacaacagtgcggctgagagcctacatgaacacccctggactgcccgtgtgtcaggatcacctggaattttgggagggcgtgttcaccggactgacccacatcgatgcccactttctgagccagaccaagcagagcggcgagaacttcccttacctggtggcttaccaggccaccgtgtgtgctagagcacaagcccctccacctagctgggaccagatgtggaagtgcctgatccggctgaagccaacactgcacggacctacacctctgctgtatagactgggcgccgtgcagaacgaagtgaccctgactcaccccgtgaccaagtacatcatgacctgcatgagcgccgacctggaagtcgtgacaagcacatgggttctcgttggcggagtgctggctgccctggctgcttattgtctgagcacaggctgcgtggtcatcgtgggcagaatctgataactcgagactgacacaaaacgtgcaacttgaaactccgcctggtctttccaggtctagaggggtaacactttgtactgtgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttacaattaatgaccctatgaatacaactgactgtttttcgctgtggtaaacgccatcaaagaggtaagagcacttttcctaccacggactgcaggaaaagctagcatgaagtttcctggcggcggacagatcgtcggcggagtttatctgctgcccagaagaggccctagactgggcgttagagccaccagaaagaccagcgagagaagccagcctagaggcagaaggcagcccattcctaaggccagaaggcccgagggaagaacatgggcccaacctggatatccctggcctctgtacggcaatgaaggctgtggatgggctggctggctgctgtctcctagaggctctagacctagctggggccctaccgatcctcggagaagaagcagaaacctgggcaaagtgatcgacaccctgacctgcggcttcgccgatctgatgggctatatccctcttgtgggagcccctcttggcggagctgctagagcacttgctcatggcgtcagagtgctggaagatggcgtgaactacgccaccggcaatctgcctggctgcagcttcagcatcttcctgtgataaccgcggccgcggtgataataggctggagcctcggtggccatgcttcttgccccttgggcctccccccagcccctcctccccttcctgcacccgtacccccgtggtctttgaataaagtctga

SEQ ID NO: 142 - DNA, 47 bp, synthetic construct
  source 1..47: mol_type = other DNA
gggaaataagagagaaaagaagagtaagaagaaatataagagccacc

SEQ ID NO: 143 - DNA, 116 bp, synthetic construct
  source 1..116: mol_type = other DNA
ccgcggtgataataggctggagcctcggtggccatgcttcttgccccttgggcctccccccagcccctcctccccttcctgcacccgtacccccgtggtctttgaataaagtctga

SEQ ID NO: 144 - DNA, 4632 bp, synthetic construct
  source 1..4632: mol_type = other DNA
gggaaataagagagaaaagaagagtaagaagaaatataagagccaccatggatgctatgaagaggggcctgtgctgcgtgctgcttctgtgtggcgctgtgtttgtgtcccctagcctggaatggcggaataccagcggcctgtacatcctgaccaacgactgccccaacagcagcatcgtgtacgaggccgacgatgtgatcctgcacacccctggctgtatcccttgcgtgcaggacggcaataccagcacctgttggacaagcgtgtcccctacagtggccgtcagatatgtgggagccaccaccgccagcatcagatctcatgtggatctgctcgtgggcgctgccacactgtgttctgctctgtacgtgggcgatatgtgtggcgccgtgtttctcgttggccaggccttcaccttcagacccagacggcatcagaccgtgcagacctgcaactgctctctgtatcctggccacctgaccggccatagaatggcctgggacatgatgatgaactggtccccagccgtcggaatggtggtggcccatgttctgagaatgccccagaccgtgttcgacatcattgccggcgctcactggggaattctggccggccttgcctactacagcatgcaaggcaactgggccaaagtggccatcattatggtcatgttcagcggcgtggacgccgagacacatacaacaggtggaaccgccgccagaaacgcctttacactgaccggactgttcacccagggcgccagacagaagctggaactgatcaacaccaacggcagctggcacatcaaccggacagccctgaactgcaacgagagcctgaacaccggctttatcgccggcctgttctacctgcacaagttcaacagcaccggctgtcccgagagactgagcagctgcaagcccatcacattcttcagacaaggctggggcagcctgaccgacgccaatattacaggccccagcgacgacaagccctactgctggcattatgcccctagaccttgcgaggtggtgcccgctctgaatgtttgtggccccgtgtactgcttcaccccatctccagtggtcgtgggcaccacagatagacagggcgtgccaacatacacctggggcgagaacgagacagacgtgttcctgctgagaagcctgaggcctccatctggccagtggttcggctgcacctggatgaatagcacaggcttcgtgaaaacctgtggcgcccctccttgcgacatatatggcggcggaggcaacagatgcaacgagtccgatctgttctgccccaccgactgcttcagaaagcaccctgaggccacctacagcagatgtggtgctggaccttggctgacccctagatgcctggtggactacccctacagactgtggcactacccttgcaccgtgaacttcaccctgttcaaagtgcggatgttcgtcggcggcttcgagcacagattcaccgccgcctgtaattggaccagaggcgagagatgcaacatcgaggaccgggacagaagcgagcagcaccctctgctgcacagcacaacagagctggccatcctgccttgcagcttcacacctatgcctgctctgagcaccggcctgatccatctgcaccagaacatcgtggacgtgcagtacctgtacggcgtcggaagcggagttgttggatgggccctgagatgggagttcgtggtcctggtgtttctgctgctggccgatgccagagtgtgtgtggctctgtggctgatgctgatgatctctcaggccgaagcctagtaactcgaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccgctagcatgagctaccaagtgggctatctgcacgcccctacaggcagcggcaagagcacaaaagtgcctgccgcttatgtggcccagggctacaatgtgctggtgctgaatcctagcgtggccgccacactcggcttcggcagctttatgtctagagcctacggcatcgaccccaacatcagaaccggcaacagaaccgtgaccaccggcgccaagctgacctactctacctacggcaagttcctggccgatggcggatgttctggcggagcctacgatgtgatcatctgcgacgaatgtcacgcccaggacgccacatctatcctcggcattggcaccgtgctggaccaggctgaaacagccggtgtcagactgacagtgctggccacagctacacctccaggcagcattacagtgccccacagcaacatcgaggaagtggcccttggaagcgagggcgagatcccattctatggcaaggccattcctatcgctctgctgaaaggcggcagacacctgatcttttgccacagcaagaagaagtgcgacgagctggcctccaagctgagaggcatgggactgaatgccgtggcctactacagaggcctggacgtgtccgtgattcctacagctggggatgtcgtcgtgtgcgccacagatgctctgatgaccggcttcaccggcgacttcgacagcgtgatcgattgcaacgtggccgtggaacagtacgtggacttctctctggaccccaccttcagcatcgagacaagaacagcccctcaggacgccgtgtctcggagtcagagaagaggcagaacaggcagaggccggctgggcacatacagatatgtgacacccggcgaaagacccagcggcatgtttgattctgtggtgctgtgcgagtgctacgacgccggatgctcttggtacgatctgcagcctgccgagacaaccgttcggctgagagcctacctgagcacacctggactgcctgtgtgtcaggaccacctggatttctgggagagcgtgttcaccggactgacccacatcgacgcccactttctgagccagacaaagcagcagggcctgaacttcagctacctgacagcctaccaggccaccgtgtgtgctagagcacaagcccctccacctagctgggacgagatgtggaagtgtctcgtgcggctgaagccaacactgcacggacctacacctctgctgtacagactgggccccgtgcagaacgagatctgtctgacacaccccatcaccaagtacatcatggcctgcatgagcgccgacctggaagtgaccacaagcacatgggttctgctcggcggagtgctggctgctctggctgcatattgtctgagcgtgggctgcgttgtgatcgtgggacacatctgataactcgagactgacacaaaacgtgcaacttgaaactccgcctggtctttccaggtctagaggggtaacactttgtactgtgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttacaattaatgaccctatgaatacaactgactgtttttcgctgtggtaaacgccatcaaagaggtaagagcacttttcctaccacggactgcaggaaaagctagcatggccgccattctgagaaggcatgttggacctggcgaaggcgctgtgcagtggatgaatagactgatcgcctttgccagccggggcaaccatgtgtccccaacacactatgtgcctgagtccgatgccgccgctagagtgacagctctgctgtaatgaccgcggccgcggtgataataggctggagcctcggtggccatgcttcttgccccttgggcctccccccagcccctcctccccttcctgcacccgtacccccgtggtctttgaataaagtctga

SEQ ID NO: 145 - skipped

SEQ ID NO: 146 - DNA, 4640 bp, synthetic construct
  source 1..4640: mol_type = other DNA
gggaaataagagagaaaagaagagtaagaagaaatataagagccaccatggatgctatgaagaggggcctgtgctgcgtgctgcttctgtgtggcgctgtgtttgtgtcccctagcgccgaagtgaagaacatcagcaccggctacatggtcaccaacgactgcaccaacgatagcatcacctggcagctgcaggccgctgtgcttcatgtgcctggatgtgtgccctgcgagaaagtgggcaatgccagccagtgttggatccccgtgtctcccaatgtggctgttcaaagacctggcgctctgacccagggcctgagaacccacatcgacatggtggtcatgtccgccacactgtgcagcgctctgtatgtgggagatctgtgtggcggcgtgatgctggccgctcagatgtttatcgtgtcccctcagcaccattggttcgtgcaggactgcaactgcagcatctaccccggcacaatcaccggccatagaatggcctgggacatgatgatgaattggagccccaccgccaccatgatcctggcctatgctatgagagtgcccgaagtgatcatcgacatcatctctggcgcccactggggcgtgatgtttggactggcttacttcagcatgcaaggcgcctgggccaaagtggtcgtgattctgcttctggctgctggcgtggacgccagaacacacacagttggaggaagcgctgcccagaccacaggcagactgaccagcctgtttgacatgggccccagacagaagatccagctggtcaacaccaacggcagctggcacatcaaccggacagccctgaactgcaacgacagcctgcacaccggctttatcgctagcctgttctacacccacagcttcaacagcagcggctgccccgaaagaatgagcgcctgtagatccatcgaggccttcagagttggatggggagccctgcagtacgaggacaacgtgaccaatcctgaggacatgcggccctactgctggcactatcctccaagacagtgcggcgtggtgtctgccaaaacagtgtgtggccccgtgtactgcttcaccccatctccagtggttgtgggcaccaccgatagactgggagcccctacttacacctggggcgagaacgagacagacgtgttcctgctgaacagcaccagacctccactcggcagttggttcggctgcacctggatgaatagctccggctacaccaagacctgtggcgcccctccatgtagaaccagagccgatttcaacgccagcaccgacctgctgtgtcccaccgactgcttcagaaagcaccccgacaccacctacctgaagtgtggctctggaccttggctgacccctagatgcctgatcgactacccctaccggctgtggcactacccctgcaccgtgaactacaccatcttcaagatccgtatgtacgtcggcggcgtggaacacagactgactgccgcctgcaatttcaccagaggcgacagatgcaacctcgaggacagagacagatcccagctgagccctctgctgcactctacaaccgagtgggccatcctgccttgcagctactctgatctgcctgctctgagcacaggcctgctgcatctgcaccagaacatcgtggacgtgcagtttatgtacggactgagccccgcactgaccaagtacatcgtcagatgggagtgggttatcctgctgttcctcctgctggccgatgccagagtgtgtgcctgtctgtggatgctgatcctgctcggacaggccgaagcttagtaactcgaggcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaaccgctagcatgacctaccaagtgggctatctgcacgcccctacaggcagcggcaagagcacaaaagtgcctgtggcctatgccgctcagggctacaaagtgctggtgctgaatccctctgtggccgccacacttggctttggcgcctacatgtctaaggcccacggcatcaaccccaacatcagaacaggcgtgcggaccgtgacaacaggcgaggccatcacctactctacctacggcaagttcctggccgatggcggatgtgctggcggagcctacgatatcatcatctgcgacgagtgccacgccgtggatgccacaacaatcctcggcatcggcaccgtgctggatcaggctgaaacagccggcgtcagactgacagtgctggccacagctacacctccaggctctgtgaccacacctcatcctaacatcgaggaagtggccctgggacaagagggcgagatcccattctacggcagagccattcctctgagctacatcaaaggcggcagacacctgatcttttgccacagcaagaagaagtgcgacgagctggccgctgctctgagaggcatgggactgaatgccgtggcctactacagaggactggacgtgtccatcattccaacacagggcgacgtggtggtggtcgccacagatgctctgatgacaggctacaccggcgacttcgacagcgtgatcgactgcaatgtggccgtgacacaggtggtggacttctccctggatcctaccttcaccatcaccacacagaccgtgcctcaggacgccgtgtctagatctcagagaagaggcagaaccggcagaggccggctgggcatctacagatatgtgtccacaggcgagcgcgccagcggcatgtttgattctgtggtgctgtgcgagtgctacgacgctggtgcagcttggtacgaactgacccctgccgaaacaaccgtgcggctgagagcctacttcaacacacctggcctgcctgtgtgccaggaccacctggaattttgggaagccgtgttcaccggcctgacacacatcgacgcccactttctgagccagacaaagcaggccggcgagaactttgcctacctggtggcctatcaggctaccgtgtgcgccagagctaaagcccctcctccttcctgggacgtgatgtggaagtgcctgaccagactgaagcccacactcgtgggacctacacctctgctgtacagactgggcagcgtgaccaacgaagtgaccctgacacatcccgtgaccaagtatatcgccacctgtatgcaggccgacctggaagtgatgaccagcacatgggttctcgcaggcggagttctggctgccgtggctgcttattgtctggccactggctgcgtgtccattatcggcagactgtgataactcgagactgacacaaaacgtgcaacttgaaactccgcctggtctttccaggtctagaggggtaacactttgtactgtgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttacaattaatgaccctatgaatacaactgactgtttttcgctgtggtaaacgccatcaaagaggtaagagcacttttcctaccacggactgcaggaaaagctagcatgacccctattcctaccaccatcatggccaagaacgaggtgttctgcgtggaccctaccaaaggcggaaagaaggccgccagactgatcgtgtatcccgacctgggagtcagagtgtgcgagaagatggccctgtacgacgtgacccagaaactgcctcaggccgtgatgggcgccagctacggctttcagtacagctgataaccgcggccgcggtgataataggctggagcctcggtggccatgcttcttgccccttgggcctccccccagcccctcctccccttcctgcacc

SEQ ID NO: 147 - DNA, 85 bp, synthetic construct
  source 1..85: mol_type = other DNA
ccgcggtgataataggctggagcctcggtggccatgcttcttgccccttgggcctccccccagcccctcctccccttcctgcacc

SEQ ID NO: 148 - protein, 554 aa, synthetic construct
YQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAIMDMIAGAHWGVLAGIAYFSMVGNWAKVLVVLLLFAGVDAETHVTGGNAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKHPEATYSRCGSGPWITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAE

SEQ ID NO: 149 - protein, 466 aa, synthetic construct
MSFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRI

SEQ ID NO: 150 - protein, 157 aa, synthetic construct
MKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFL

SEQ ID NO: 151 - protein, 561 aa, synthetic construct
LEWRNTSGLYILTNDCPNSSIVYEADDVILHTPGCIPCVQDGNTSTCWTSVSPTVAVRYVGATTASIRSHVDLLVGAATLCSALYVGDMCGAVFLVGQAFTFRPRRHQTVQTCNCSLYPGHLTGHRMAWDMMMNWSPAVGMVVAHVLRMPQTVFDIIAGAHWGILAGLAYYSMQGNWAKVAIIMVMFSGVDAETHTTGGTAARNAFTLTGLFTQGARQKLELINTNGSWHINRTALNCNESLNTGFIAGLFYLHKFNSTGCPERLSSCKPITFFRQGWGSLTDANITGPSDDKPYCWHYAPRPCEVVPALNVCGPVYCFTPSPVVVGTTDRQGVPTYTWGENETDVFLLRSLRPPSGQWFGCTWMNSTGFVKTCGAPPCDIYGGGGNRCNESDLFCPTDCFRKHPEATYSRCGAGPWLTPRCLVDYPYRLWHYPCTVNFTLFKVRMFVGGFEHRFTAACNWTRGERCNIEDRDRSEQHPLLHSTTELAILPCSFTPMPALSTGLIHLHQNIVDVQYLYGVGSGVVGWALRWEFVVLVFLLLADARVCVALWLMLMISQAEA

SEQ ID NO: 152 - protein, 466 aa, synthetic construct
MSYQVGYLHAPTGSGKSTKVPAAYVAQGYNVLVLNPSVAATLGFGSFMSRAYGIDPNIRTGNRTVTTGAKLTYSTYGKFLADGGCSGGAYDVIICDECHAQDATSILGIGTVLDQAETAGVRLTVLATATPPGSITVPHSNIEEVALGSEGEIPFYGKAIPIALLKGGRHLIFCHSKKKCDELASKLRGMGLNAVAYYRGLDVSVIPTAGDVVVCATDALMTGFTGDFDSVIDCNVAVEQYVDFSLDPTFSIETRTAPQDAVSRSQRRGRTGRGRLGTYRYVTPGERPSGMFDSVVLCECYDAGCSWYDLQPAETTVRLRAYLSTPGLPVCQDHLDFWESVFTGLTHIDAHFLSQTKQQGLNFSYLTAYQATVCARAQAPPPSWDEMWKCLVRLKPTLHGPTPLLYRLGPVQNEICLTHPITKYIMACMSADLEVTTSTWVLLGGVLAALAAYCLSVGCVVIVGHI

SEQ ID NO: 153 - protein, 51 aa, synthetic construct
MAAILRRHVGPGEGAVQWMNRLIAFASRGNHVSPTHYVPESDAAARVTALL

SEQ ID NO: 154 - protein, 559 aa, synthetic construct
AEVKNISTGYMVTNDCTNDSITWQLQAAVLHVPGCVPCEKVGNASQCWIPVSPNVAVQRPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIISGAHWGVMFGLAYFSMQGAWAKVVVILLLAAGVDARTHTVGGSAAQTTGRLTSLFDMGPRQKIQLVNTNGSWHINRTALNCNDSLHTGFIASLFYTHSFNSSGCPERMSACRSIEAFRVGWGALQYEDNVTNPEDMRPYCWHYPPRQCGVVSAKTVCGPVYCFTPSPVVVGTTDRLGAPTYTWGENETDVFLLNSTRPPLGSWFGCTWMNSSGYTKTCGAPPCRTRADFNASTDLLCPTDCFRKHPDTTYLKCGSGPWLTPRCLIDYPYRLWHYPCTVNYTIFKIRMYVGGVEHRLTAACNFTRGDRCNLEDRDRSQLSPLLHSTTEWAILPCSYSDLPALSTGLLHLHQNIVDVQFMYGLSPALTKYIVRWEWVILLFLLLADARVCACLWMLILLGQAEA

SEQ ID NO: 155 - protein, 466 aa, synthetic construct
MTYQVGYLHAPTGSGKSTKVPVAYAAQGYKVLVLNPSVAATLGFGAYMSKAHGINPNIRTGVRTVTTGEAITYSTYGKFLADGGCAGGAYDIIICDECHAVDATTILGIGTVLDQAETAGVRLTVLATATPPGSVTTPHPNIEEVALGQEGEIPFYGRAIPLSYIKGGRHLIFCHSKKKCDELAAALRGMGLNAVAYYRGLDVSIIPTQGDVVVVATDALMTGYTGDFDSVIDCNVAVTQVVDFSLDPTFTITTQTVPQDAVSRSQRRGRTGRGRLGIYRYVSTGERASGMFDSVVLCECYDAGAAWYELTPAETTVRLRAYFNTPGLPVCQDHLEFWEAVFTGLTHIDAHFLSQTKQAGENFAYLVAYQATVCARAKAPPPSWDVMWKCLTRLKPTLVGPTPLLYRLGSVTNEVTLTHPVTKYIATCMQADLEVMTSTWVLAGGVLAAVAAYCLATGCVSIIGRL

SEQ ID NO: 156 - protein, 66 aa, synthetic construct
MTPIPTTIMAKNEVFCVDPTKGGKKAARLIVYPDLGVRVCEKMALYDVTQKLPQAVMGASYGFQYS

SEQ ID NO: 157 - DNA, 47 bp, synthetic construct
  source 1..47: mol_type = other DNA
gggagatcagagagaaaagaagagtaagaagaaatataagagccacc

SEQ ID NO: 158 - DNA, 145 bp, synthetic construct
  source 1..145: mol_type = other DNA
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcaac

SEQ ID NO: 159 - DNA, 33 bp, synthetic construct
  source 1..33: mol_type = other DNA
gggagacaagcggcaccggacgggaaagccacc

SEQ ID NO: 160 - protein, 20 aa, synthetic construct
YHADYYKQRLIHDVEMNPGP

SEQ ID NO: 161 - protein, 20 aa, synthetic construct
HYAGYFADLLIHDIETNPGP

SEQ ID NO: 162 - protein, 20 aa, synthetic construct
QCTNYALLKLAGDVESNPGP

SEQ ID NO: 163 - protein, 19 aa, synthetic construct
ATNFSLLKQAGDVEENPGP

SEQ ID NO: 164 - protein, 19 aa, synthetic construct
AARQMLLLLSGDVETNPGP

SEQ ID NO: 165 - protein, 20 aa, synthetic construct
RAEGRGSLLTCGDVEENPGP

SEQ ID NO: 166 - protein, 20 aa, synthetic construct
TRAEIEDELIRAGIESNPGP

SEQ ID NO: 167 - protein, 20 aa, synthetic construct
TRAEIEDELIRADIESNPGP

SEQ ID NO: 168 - protein, 20 aa, synthetic construct
AKFQIDKILISGDVELNPGP

SEQ ID NO: 169 - protein, 20 aa, synthetic construct
SSIIRTKMLVSGDVEENPGP

SEQ ID NO: 170 - protein, 20 aa, synthetic construct
CDAQRQKLLLSGDIEQNPGP

SEQ ID NO: 171 - protein, 18 aa, synthetic construct
EGRGSLLTCGDVEENPGP

SEQ ID NO: 172 - protein, 20 aa, synthetic construct
YPIDFGGFLVKADSEFNPGP

SEQ ID NO: 173 - protein, 19 aa, synthetic construct
LLNFDLLKLAGDVESNPGP

SEQ ID NO: 174 - protein, 19 aa, synthetic construct
TLNFDLLKLAGDVESNPGP

SEQ ID NO: 175 - protein, 14 aa, synthetic construct
LLKLAGDVESNPGP

SEQ ID NO: 176 - protein, 17 aa, synthetic construct
NFDLLKLAGDVESNPGP

SEQ ID NO: 177 - protein, 20 aa, synthetic construct
QLLNFDLLKLAGDVESNPGP

SEQ ID NO: 178 - protein, 24 aa, synthetic construct
APVKQTLNFDLLKLAGDVESNPGP

SEQ ID NO: 179 - protein, 40 aa, synthetic construct
VTELLYRMKRAETYCPRPLLAIHPTEARHKQKIVAPVKQT

SEQ ID NO: 180 - protein, 18 aa, synthetic construct
LNFDLLKLAGDVESNPGP

SEQ ID NO: 181 - protein, 40 aa, synthetic construct
LLAIHPTEARHKQKIVAPVKQTLNFDLLKLAGDVESNPGP

SEQ ID NO: 182 - protein, 33 aa, synthetic construct
EARHKQKIVAPVKQTLNFDLLKLAGDVESNPGP

SEQ ID NO: 183 - protein, 22 aa, synthetic construct
VKQTLNFDLLKLAGDVESNPGP

SEQ ID NO: 184-185 - skipped (2 entries)

SEQ ID NO: 186 - protein, 6 aa, synthetic construct
  VARIANT 2: note = X is Leu or Gln
PXGMTS

SEQ ID NO: 187 - protein, 5 aa, synthetic construct
  VARIANT 2: note = X is Leu or Gln
PXGMT

SEQ ID NO: 188 - protein, 7 aa, Tobacco etch virus
ENLYTQS

SEQ ID NO: 189 - protein, 5 aa, synthetic construct
DDDDK

SEQ ID NO: 190 - protein, 4 aa, synthetic construct
LVPR

SEQ ID NO: 191 - protein, 8 aa, synthetic construct
LEVLFQGP

SEQ ID NO: 192 - protein, 10 aa, synthetic construct
CGLVPAGSGP

SEQ ID NO: 193 - protein, 12 aa, synthetic construct
SLLKSRMVPNFN

SEQ ID NO: 194 - protein, 12 aa, synthetic construct
SLLIARRMPNFN

SEQ ID NO: 195 - protein, 12 aa, synthetic construct
SKLVQASASGVN

SEQ ID NO: 196 - protein, 12 aa, synthetic construct
SSYLKASDAPDN

SEQ ID NO: 197 - protein, 12 aa, synthetic construct
RPKPQQFFGLMN

SEQ ID NO: 198 - protein, 12 aa, synthetic construct
SLRPLALWRSFN

SEQ ID NO: 199 - protein, 12 aa, synthetic construct
SPQGIAGQRNFN

SEQ ID NO: 200 - protein, 14 aa, synthetic construct
DVDERDVRGFASFL

SEQ ID NO: 201 - protein, 12 aa, synthetic construct
SLPLGLWAPNFN

SEQ ID NO: 202 - protein, 12 aa, synthetic construct
SLLIFRSWANFN

SEQ ID NO: 203 - protein, 12 aa, synthetic construct
SGVVIATVIVIT

SEQ ID NO: 204 - protein, 12 aa, synthetic construct
SLGPQGIWGQFN

SEQ ID NO: 205 - protein, 12 aa, synthetic construct
KKSPGRVVGGSV

SEQ ID NO: 206 - protein, 12 aa, synthetic construct
PQGLLGAPGILG

SEQ ID NO: 207 - protein, 31 aa, synthetic construct
HGPEGLRVGFYESDVMGRGHARLVHVEEPHT

SEQ ID NO: 208 - protein, 12 aa, synthetic construct
GPQGLAGQRGIV

SEQ ID NO: 209 - protein, 12 aa, synthetic construct
GGSGQRGRKALE

SEQ ID NO: 210 - protein, 12 aa, synthetic construct
SLSALLSSDIFN

SEQ ID NO: 211 - protein, 12 aa, synthetic construct
SLPRFKIIGGFN

SEQ ID NO: 212 - protein, 12 aa, synthetic construct
SLLGIAVPGNFN

SEQ ID NO: 213 - protein, 12 aa, synthetic construct
FFKNIVTPRTPP

SEQ ID NO: 214 - DNA, 69 bp, synthetic construct
  source 1..69: mol_type = other DNA
atggacgcaatgaaacggggtctgtgctgcgtactcttgctttgtggagctgtgttcgtgtcaccttct

SEQ ID NO: 215 - DNA, 75 bp, synthetic construct
  source 1..75: mol_type = other DNA
atgatgccatcctcagtctcatggggtattttgctcttggcgggtctgtgctgtctcgtgccggtgtcgctcgca

SEQ ID NO: 216 - protein, 24 aa, synthetic construct
MMPSSVSWGILLAGLCCLVPVSLA

SEQ ID NO: 217 - DNA, 78 bp, synthetic construct
  source 1..78: mol_type = other DNA
atggcgacgccgctgcctccgccctccccgcggcacctgcggctgctgcggctgctgctctccgccctcgtcctcggc

SEQ ID NO: 218 - protein, 22 aa, synthetic construct
MATPLPPPSPRHLRLLRLLLSG

SEQ ID NO: 219 - protein, 35 aa, synthetic construct
  VARIANT 7: note = X is Ala or Val
  VARIANT 10: note = X is Ala or Val
  VARIANT 11: note = X is Ala or Ser
  VARIANT 15: note = X is Lys or Asn
  VARIANT 31: note = X is Ala or Ser
  VARIANT 32: note = X is Tyr or Phe
  VARIANT 33: note = X is Met or Leu
  VARIANT 35: note = X is Lys or Arg
KSTKVPXAYXXQGYXVLVLNPSVAATLGFGXXXSX

SEQ ID NO: 220 - protein, 35 aa, synthetic construct
KSTKVPVAYAAQGYKVLVLNPSVAATLGFGAYLSK

SEQ ID NO: 221 - protein, 35 aa, synthetic construct
KSTKVPAAYASQGYKVLVLNPSVAATLGFGAYMSK

SEQ ID NO: 222 - protein, 35 aa, synthetic construct
KSTKVPAAYVAQGYNVLVLNPSVAATLGFGSFMSR

SEQ ID NO: 223 - protein, 35 aa, synthetic construct
KSTKVPAAYAAQGYKVLVLNPSVAATLGFGAYMSR

SEQ ID NO: 224 - protein, 35 aa, synthetic construct
KSTKVPAAYASQGYKVLVLNPSVAATLGFGSYMSK

SEQ ID NO: 225 - protein, 10 aa, synthetic construct
  VARIANT 3: note = X is Ala or Val
  VARIANT 4: note = X is Ala or Ser
  VARIANT 8: note = X is Lys or Asn
AYXXQGYXVL

SEQ ID NO: 226 - protein, 11 aa, synthetic construct
  VARIANT 7: note = X is Ala or Ser
  VARIANT 8: note = X is Tyr or Phe
  VARIANT 9: note = X is Met or Leu
  VARIANT 11: note = X is Lys or Arg
ATLGFGXXXSX

SEQ ID NO: 227 - protein, 10 aa, synthetic construct
AYAAQGYKVL

SEQ ID NO: 228 - protein, 10 aa, synthetic construct
AYASQGYKVL

SEQ ID NO: 229 - protein, 10 aa, synthetic construct
AYVAQGYNVL

SEQ ID NO: 230 - protein, 11 aa, synthetic construct
ATLGFGAYMSK

SEQ ID NO: 231 - protein, 15 aa, synthetic construct
VLVLNPSVAATLGFG

SEQ ID NO: 232 - protein, 30 aa, synthetic construct
PAAYAAQGYKVLVLNPSVAATLGFGAYMSK

SEQ ID NO: 233 - protein, 30 aa, synthetic construct
KSTKVPAAYAAQGYKVLVLNPSVAATLGFG

SEQ ID NO: 234 - protein, 50 aa, synthetic construct
  VARIANT 4: note = X is Leu or Val
  VARIANT 16: note = X is Ala or Gly
  VARIANT 20: note = X is Thr or Ser
  VARIANT 32: note = X is Pro or Arg
  VARIANT 36: note = X is Ala or Asp
  VARIANT 37: note = X is Arg or Ala
  VARIANT 38: note = X is Arg, Gln, or Ser
  VARIANT 39: note = X is Ser or Pro
  VARIANT 40: note = X is Glu, Thr, or Gln
  VARIANT 42: note = X is Arg or Lys
  VARIANT 43: note = X is Ser, Thr, His, or Ala
  VARIANT 45: note = X is Ala or Gly
  VARIANT 46: note = X is Gln or Lys
GVYXLPRRGPRLGVRXTRKXSERSQPRGRRQXIPKXXXXXGXXWXXPGYP

SEQ ID NO: 235 - protein, 50 aa, synthetic construct
  VARIANT 4: note = X is Leu or Val
  VARIANT 36: note = X is Ala or Asp
  VARIANT 38: note = X is Arg, Gln, or Ser
  VARIANT 39: note = X is Ser or Pro
  VARIANT 40: note = X is Glu, Thr, or Gln
  VARIANT 42: note = X is Arg or Lys
  VARIANT 43: note = X is Ser, Thr, His, or Ala
  VARIANT 45: note = X is Ala or Gly
  VARIANT 46: note = X is Gln or Lys
GVYXLPRRGPRLGVRATRKTSERSQPRGRRQPIPKXRXXXGXXWXXPGYP

SEQ ID NO: 236 - protein, 50 aa, synthetic construct
GVYLLPRRGPRLGVRATRKTSERSQPRGRRQPIPKARRPEGRTWAQPGYP

SEQ ID NO: 237 - protein, 50 aa, synthetic construct
GVYLLPRRGPRLGVRATRKTSERSQPRGRRQPIPKARRPEGRAWAQPGYP

SEQ ID NO: 238 - protein, 50 aa, synthetic construct
GVYLLPRRGPRLGVRATRKTSERSQPRGRRQPIPKDRRSTGKSWGKPGYP

SEQ ID NO: 239 - protein, 50 aa, synthetic construct
GVYVLPRRGPRLGVRATRKTSERSQPRGRRQPIPKARRSEGRSWAQPGYP

SEQ ID NO: 240 - protein, 50 aa, synthetic construct
GVYLLPRRGPRLGVRATRKTSERSQPRGRRQPIPKARRPTGRSWGQPGYP

SEQ ID NO: 241 - protein, 50 aa, synthetic construct
GVYLLPRRGPRLGVRATRKTSERSQPRGRRQPIPKARQPTGRHWAQPGYP

SEQ ID NO: 242 - protein, 50 aa, synthetic construct
GVYLLPRRGPRLGVRTTRKSSERSQPRGRRQRIPKAASSQGKAWGKPGYP

SEQ ID NO: 243 - protein, 25 aa, synthetic construct
RRGPRLGVRATRKTSERSQPRGRRQ

SEQ ID NO: 244 - protein, 25 aa, synthetic construct
RRGPRLGVRGTRKSSERSQPRGRRQ

SEQ ID NO: 245 - protein, 24 aa, synthetic construct
RATRKTSERSQPRGRRQPIPKARR

SEQ ID NO: 246 - protein, 24 aa, synthetic construct
RGTRKSSERSQPRGRRQRIPKAAQ

SEQ ID NO: 247 - protein, 24 aa, synthetic construct
RATRKTSERSQPRGRRQPIPKARQ

SEQ ID NO: 248 - protein, 24 aa, synthetic construct
RATRKTSERSQPRGRRQPIPKDRR

SEQ ID NO: 249 - protein, 20 aa, synthetic construct
RRQPIPKARRSEGRSWAQPG

SEQ ID NO: 250 - protein, 20 aa, synthetic construct
RRQPIPKARPSEGRTWAQPG

SEQ ID NO: 251 - protein, 20 aa, synthetic construct
RRQPIPKARRPEGRAWAQPG

SEQ ID NO: 252 - protein, 20 aa, synthetic construct
RRQPIPKDRRSTGKSWGKPG

SEQ ID NO: 253 - protein, 20 aa, synthetic construct
RRQPIPKARRPTGRSWGQPG

SEQ ID NO: 254 - protein, 20 aa, synthetic construct
RRQPIPKARQPTGRHWAQPG

SEQ ID NO: 255 - protein, 20 aa, synthetic construct
RRQRIPKAASSQGKAWGKPG

SEQ ID NO: 256 - protein, 23 aa, synthetic construct
DVVVVATDALMTGFTGDFDSVID

SEQ ID NO: 257 - protein, 23 aa, synthetic construct
  VARIANT 5: note = X is Val or Cys
  VARIANT 6: note = X is Ala or Ser
  VARIANT 14: note = X is Phe or Tyr
DVVVXXTDALMTGXTGDFDSVID

SEQ ID NO: 258 - protein, 23 aa, synthetic construct
DVVVVATDALMTGYTGDFDSVID

SEQ ID NO: 259 - protein, 23 aa, synthetic construct
DVVVCATDALMTGFTGDFDSVID

SEQ ID NO: 260 - protein, 23 aa, synthetic construct
DVVVCSTDALMTGFTGDFDSVID

SEQ ID NO: 261 - protein, 23 aa, synthetic construct
DVVVCATDALMTGYTGDFDSVID

SEQ ID NO: 262 - protein, 9 aa, synthetic construct
ATDALMTGF

SEQ ID NO: 263 - protein, 9 aa, synthetic construct
STDALMTGF

SEQ ID NO: 264 - protein, 9 aa, synthetic construct
ATDALMTGY

SEQ ID NO: 265 - protein, 9 aa, synthetic construct
GFTGDFDSV

SEQ ID NO: 266 - protein, 9 aa, synthetic construct
GYTGDFDSV

SEQ ID NO: 267 - protein, 15 aa, synthetic construct
ATDALMTGFTGDFDS

SEQ ID NO: 268 - protein, 15 aa, synthetic construct
STDALMTGFTGDFDS

SEQ ID NO: 269 - protein, 15 aa, synthetic construct
ATDALMTGYTGDFDS

SEQ ID NO: 270 - protein, 15 aa, synthetic construct
ALMTGFTGDFDSVID

SEQ ID NO: 271 - protein, 15 aa, synthetic construct
ALMTGYTGDFDSVID

SEQ ID NO: 272 - protein, 27 aa, synthetic construct
LEQIKGGRHLIFCHSKKKCDELAAKLT

SEQ ID NO: 273 - protein, 27 aa, synthetic construct
  VARIANT 1: note = X is Leu or Ile
  VARIANT 2: note = X is Glu, Ala, Ser, Val, or Gln
  VARIANT 3: note = X is Gln, Thr, Tyr, Phe, or Leu
  VARIANT 4: note = X is Ile or Leu
  VARIANT 22: note = X is Leu or Ile
  VARIANT 24: note = X is Ala, Lys, or Ser
  VARIANT 25: note = X is Lys, Gln, or Ala
  VARIANT 27: note = X is Thr, Arg, or Ser
XXXXKGGRHLIFCHSKKKCDEXAXXLX

SEQ ID NO: 274 - protein, 27 aa, synthetic construct
LEVIKGGRHLIFCHSKKKCDELAAKLV

SEQ ID NO: 275 - protein, 27 aa, synthetic construct
IETIKGGRHLIFCHSKKKCDELAAKLS

SEQ ID NO: 276 - protein, 27 aa, synthetic construct
LSYIKGGRHLIFCHSKKKCDELAAALR

SEQ ID NO: 277 - protein, 26 aa, synthetic construct
LAFIKGGRHLIFCHSKKKCDELAALR

SEQ ID NO: 278 - protein, 27 aa, synthetic construct
IAQLKGGRHLIFCHSKKKCDEIASKLR

SEQ ID NO: 279 - protein, 26 aa, synthetic construct
LELIKGGRHLIFCHSKKKCDELAQLT

SEQ ID NO: 280 - protein, 27 aa, synthetic construct
  VARIANT 2: note = X can be any naturally occurring amino acid
LXLIKGGRHLIFCHSKKKCDELAKQLT

SEQ ID NO: 281 - protein, 27 aa, synthetic construct
LEYIKGGRHLIFCHSKKKCDELAKQLT

SEQ ID NO: 282 - protein, 27 aa, synthetic construct
LQHIKGGRHLIFCHSKKKCDELAGKLT

SEQ ID NO: 283 - protein, 9 aa, synthetic construct
LIFCHSKKK

SEQ ID NO: 284 - protein, 9 aa, synthetic construct
HSKKKCDEL

SEQ ID NO: 285 - protein, 16 aa, synthetic construct
KGGRHLIFCHSKKKCD

SEQ ID NO: 286 - protein, 11 aa, synthetic construct
GRHLIFCHSKK

SEQ ID NO: 287 - protein, 35 aa, synthetic construct
ILRRHVGPGEGAVQWMNRLIAFASRGNHVSPTHYV

SEQ ID NO: 288 - protein, 35 aa, synthetic construct
  VARIANT 1: note = X is Ile or Val
  VARIANT 2: note = X is Leu or Ile
  VARIANT 3: note = X is Arg or Lys
  VARIANT 6: note = X is Val, Ile, or Thr
  VARIANT 8: note = X is Pro, Gln, or Thr
  VARIANT 9: note = X is Gly, Ala, or Ser
  VARIANT 12: note = X is Ala or Val
  VARIANT 13: note = X is Val or Thr
  VARIANT 30: note = X is Ser or Ala
  VARIANT 35: note = X is Val or Ile
XXXRHXGXXEGXXQWMNRLIAFASRGNHVXPTHYX

SEQ ID NO: 289 - protein, 35 aa, synthetic construct
  VARIANT 1: note = X is Ile or Val
  VARIANT 6: note = X is Leu or Ile
  VARIANT 8: note = X is Arg or Lys
  VARIANT 9: note = X is Gly or Ala
  VARIANT 13: note = X is Val or Thr
  VARIANT 30: note = X is Ser or Ala
XLRRHXGXXEGAXQWMNRLIAFASRGNHVXPTHYV

SEQ ID NO: 290 - protein, 35 aa, synthetic construct
ILRRHVGPGEGAVQWMNRLIAFASRGNHVAPTHYV

SEQ ID NO: 291 - protein, 35 aa, synthetic construct
ILRRHVGQGEGAVQWMNRLIAFASRGNHVAPTHYV

SEQ ID NO: 292 - protein, 35 aa, synthetic construct
VLRRHIGPGEGAVQWMNRLIAFASRGNHVSPTHYV

SEQ ID NO: 293 - protein, 35 aa, synthetic construct
ILRRHVGPAEGATQWMNRLIAFASRGNHVSPTHYV

SEQ ID NO: 294 - protein, 35 aa, synthetic construct
IIKRHTGTSEGVTQWMNRLIAFASRGNHVSPTHYI

SEQ ID NO: 295 - protein, 16 aa, synthetic construct
EGAVQWMNRLIAFASR

SEQ ID NO: 296 - protein, 16 aa, synthetic construct
EGATQWMNRLIAFASR

SEQ ID NO: 297 - protein, 16 aa, synthetic construct
EGVTQWMNRLIAFASR

SEQ ID NO: 298 - protein, 9 aa, synthetic construct
WMNRLIAFA

SEQ ID NO: 299 - protein, 15 aa, synthetic construct
RHVGPGEGAVQWMNR

SEQ ID NO: 300 - protein, 15 aa, synthetic construct
RHVGQGEGAVQWMNR

SEQ ID NO: 301 - protein, 15 aa, synthetic construct
RHIGPGEGAVQWMNR

SEQ ID NO: 302 - protein, 15 aa, synthetic construct
RHVGPAEGATQWMNR

SEQ ID NO: 303 - protein, 15 aa, synthetic construct
RHTGTSEGVTQWMNR

SEQ ID NO: 304 - protein, 20 aa, synthetic construct
HVGPGEGAVQWMNRLIAFAS

SEQ ID NO: 305 - protein, 20 aa, synthetic construct
HVGQGEGAVQWMNRLIAFAS

SEQ ID NO: 306 - protein, 20 aa, synthetic construct
HIGPGEGAVQWMNRLIAFAS

SEQ ID NO: 307 - protein, 20 aa, synthetic construct
HVGPAEGATQWMNRLIAFAS

SEQ ID NO: 308 - protein, 20 aa, synthetic construct
HTGTSEGVTQWMNRLIAFAS

SEQ ID NO: 309 - protein, 20 aa, synthetic construct
GAVQWMNRLIAFASRGNHVS

SEQ ID NO: 310 - protein, 20 aa, synthetic construct
GAVQWMNRLIAFASRGNHVA

SEQ ID NO: 311 - protein, 20 aa, synthetic construct
GATQWMNRLIAFASRGNHVS

SEQ ID NO: 312 - protein, 20 aa, synthetic construct
GVTQWMNRLIAFASRGNHVS

SEQ ID NO: 313 - protein, 42 aa, synthetic construct
GTEGEIPFYGKAIPLEQIKGGRHLIFCHSKKKCDELAAKLTG

SEQ ID NO: 314 - protein, 52 aa, synthetic construct
  VARIANT 1: note = X is Gly, Pro, or Ser
  VARIANT 2: note = X is Thr, Asn, Gln, His, or Ser
  VARIANT 3: note = X is Glu, Thr, or Asp
  VARIANT 11: note = X is Lyr or Arg
  VARIANT 15: note = X is Leu or Ile
  VARIANT 16: note = X is Glu, Ala, Ser, or Gln
  VARIANT 17: note = X is Gln, Thr, Tyr, Phe, or Leu
  VARIANT 18: note = X is Ile or Leu
  VARIANT 36: note = X is Leu or Ile
  VARIANT 38: note = X is Ala, Lys, or Ser
  VARIANT 39: note = X is Lys, Gln, or Ala
  VARIANT 41: note = X is Thr, Arg, or Ser
  VARIANT 42: note = X is Gly or Ser
  VARIANT 43..52: note = Each copy of Lys at positions 43 to 52 is either present or absent such that the number of copies of Lys ranges from 2 to 10
XXXGEIPFYGXAIPXXXXKGGRHLIFCHSKKKCDEXAXXLXXKKKKKKKKKK

SEQ ID NO: 315 - protein, 45 aa, synthetic construct
  VARIANT 1: note = X is Gly, Pro, or Ser
  VARIANT 2: note = X is Thr, Asn, Gln, His, or Ser
  VARIANT 3: note = X is Glu, Thr, or Asp
  VARIANT 11: note = X is Lyr or Arg
  VARIANT 15: note = X is Leu or Ile
  VARIANT 16: note = X is Glu, Ala, Ser, or Gln
  VARIANT 17: note = X is Gln, Thr, Tyr, Phe, or Leu
  VARIANT 18: note = X is Ile or Leu
  VARIANT 36: note = X is Leu or Ile
  VARIANT 38: note = X is Ala, Lys, or Ser
  VARIANT 39: note = X is Lys, Gln, or Ala
  VARIANT 41: note = X is Thr, Arg, or Ser
  VARIANT 42: note = X is Gly or Ser
XXXGEIPFYGXAIPXXXXKGGRHLIFCHSKKKCDEXAXXLXXKKK

SEQ ID NO: 316 - protein, 42 aa, synthetic construct
  VARIANT 1: note = X is Gly, Pro, or Ser
  VARIANT 2: note = X is Thr, Asn, Gln, His, or Ser
  VARIANT 3: note = X is Glu, Thr, or Asp
  VARIANT 11: note = X is Lyr or Arg
  VARIANT 15: note = X is Leu or Ile
  VARIANT 16: note = X is Glu, Ala, Ser, or Gln
  VARIANT 17: note = X is Gln, Thr, Tyr, Phe, or Leu
  VARIANT 18: note = X is Ile or Leu
  VARIANT 36: note = X is Leu or Ile
  VARIANT 38: note = X is Ala, Lys, or Ser
  VARIANT 39: note = X is Lys, Gln, or Ala
  VARIANT 41: note = X is Thr, Arg, or Ser
  VARIANT 42: note = X is Gly or Ser
XXXGEIPFYGXAIPXXXXKGGRHLIFCHSKKKCDEXAXXLXX

SEQ ID NO: 317 - protein, 42 aa, synthetic construct
STTGEIPFYGKAIPLEVIKGGRHLIFCHSKKKCDELAAKLVA

SEQ ID NO: 318 - protein, 42 aa, synthetic construct
SNTGEIPFYGKAIPIETIKGGRHLIFCHSKKKCDELAAKLSG

SEQ ID NO: 319 - protein, 42 aa, synthetic construct
GQEGEIPFYGRAIPLSYIKGGRHLIFCHSKKKCDELAAALRG

SEQ ID NO: 320 - protein, 41 aa, synthetic construct
GHEGEIPFYGKAIPLAFIKGGRHLIFCHSKKKCDELAALRG

SEQ ID NO: 321 - protein, 42 aa, synthetic construct
GSEGEIPFYGKAIPIAQLKGGRHLIFCHSKKKCDEIASKLRG

SEQ ID NO: 322 - protein, 41 aa, synthetic construct
PTTGEIPFYGKAIPLELIKGGRHLIFCHSKKKCDELAQLTS

SEQ ID NO: 323 - protein, 42 aa, synthetic construct
  VARIANT 16: note = X can be any naturally occurring amino acid
PSEGEIPFYGRAIPLXLIKGGRHLIFCHSKKKCDELAKQLTS

SEQ ID NO: 324 - protein, 42 aa, synthetic construct
PTTGEIPFYGKAIPLEYIKGGRHLIFCHSKKKCDELAKQLTS

SEQ ID NO: 325 - protein, 42 aa, synthetic construct
GNDGEIPFYGKAIPLQHIKGGRHLIFCHSKKKCDELAGKLTS

SEQ ID NO: 326 - protein, 11 aa, synthetic construct
  VARIANT 4: note = X is Lys or Arg
  VARIANT 8: note = X is Leu or Ile
  VARIANT 9: note = X is Glu, Ser, Ala, Gln, or Val
  VARIANT 10: note = X is Gln, Thr, Tyr, Phe, Leu, or His
  VARIANT 11: note = X is Ile or Leu
FYGXAIPXXXX

SEQ ID NO: 327 - protein, 11 aa, synthetic construct
FYGKAIPLEQI

SEQ ID NO: 328 - protein, 37 aa, synthetic construct
IPFYGKAIPLEQIKGGRHLIFCHSKKKCDELAAKLTG

SEQ ID NO: 329 - protein, 37 aa, synthetic construct
GTEGEIPFYGKAIPLEQIKGGRHLIFCHSKKKCDELA

SEQ ID NO: 330 - protein, 45 aa, synthetic construct
LNAVAYYRGLDVSVIPTSGDVVVVATDALMTGFTGDFDSVIDCNV

SEQ ID NO: 331 - protein, 45 aa, synthetic construct
LNAVAYYRGLDVSVIPTSGDVVVVATDALMTGFTGDFDSVIDKKK

SEQ ID NO: 332 - protein, 55 aa, synthetic construct
  VARIANT 1: note = X is Leu or Val
  VARIANT 2: note = X is Asn or Thr
  VARIANT 6: note = X is Tyr or Phe
  VARIANT 10: note = X is Leu or Val
  VARIANT 13: note = X is Ser or Ala
  VARIANT 14: note = X is Val or Ile
  VARIANT 17: note = X is Thr or Ala
  VARIANT 18: note = X is Ser, Gln, or Thr
  VARIANT 24: note = X is Val or Cys
  VARIANT 25: note = X is Ala or Ser
  VARIANT 33: note = X is Phe or Tyr
  VARIANT 43: note = X is Cys or Lys
  VARIANT 44: note = X is Asn or Lys
  VARIANT 45: note = X is Val or Lys
  VARIANT 46..55: note = Each copy of Lys at positions 46 to 55 is either present or absent such that the number of copies of Lys ranges from 2 to 10
XXAVAXYRGXDVXXIPXXGDVVVXXTDALMTGXTGDFDSVIDXXXKKKKKKKKKK

SEQ ID NO: 333 - protein, 48 aa, synthetic construct
  VARIANT 1: note = X is Leu or Val
  VARIANT 2: note = X is Asn or Thr
  VARIANT 6: note = X is Tyr or Phe
  VARIANT 10: note = X is Leu or Val
  VARIANT 13: note = X is Ser or Ala
  VARIANT 14: note = X is Val or Ile
  VARIANT 17: note = X is Thr or Ala
  VARIANT 18: note = X is Ser, Gln, or Thr
  VARIANT 24: note = X is Val or Cys
  VARIANT 25: note = X is Ala or Ser
  VARIANT 33: note = X is Phe or Tyr
  VARIANT 43: note = X is Cys or Lys
  VARIANT 44: note = X is Asn or Lys
  VARIANT 45: note = X is Val or Lys
XXAVAXYRGXDVXXIPXXGDVVVXXTDALMTGXTGDFDSVIDXXXKKK

SEQ ID NO: 334 - protein, 48 aa, synthetic construct
LNAVAYYRGLDVSVIPTSGDVVVVATDALMTGFTGDFDSVIDCNVKKK

SEQ ID NO: 335 - protein, 45 aa, synthetic construct
  VARIANT 1: note = X is Leu or Val
  VARIANT 2: note = X is Asn or Thr
  VARIANT 6: note = X is Tyr or Phe
  VARIANT 10: note = X is Leu or Val
  VARIANT 13: note = X is Ser or Ala
  VARIANT 14: note = X is Val or Ile
  VARIANT 17: note = X is Thr or Ala
  VARIANT 18: note = X is Ser, Gln, or Thr
  VARIANT 24: note = X is Val or Cys
  VARIANT 25: note = X is Ala or Ser
  VARIANT 33: note = X is Phe or Tyr
  VARIANT 43: note = X is Cys or Lys
  VARIANT 44: note = X is Asn or Lys
  VARIANT 45: note = X is Val or Lys
XXAVAXYRGXDVXXIPXXGDVVVXXTDALMTGXTGDFDSVIDXXX

SEQ ID NO: 336 - protein, 45 aa, synthetic construct
  VARIANT 18: note = X is Ser, Gln, or Thr
  VARIANT 24: note = X is Val or Cys
  VARIANT 33: note = X is Phe or Tyr
LNAVAYYRGLDVSVIPTXGDVVVXATDALMTGXTGDFDSVIDKKK

SEQ ID NO: 337 - protein, 42 aa, synthetic construct
LNAVAYYRGLDVSVIPTSGDVVVVATDALMTGFTGDFDSVID

SEQ ID NO: 338 - protein, 48 aa, synthetic construct
ILRRHVGPGEGAVQWMNRLIAFASRGNHVSPTHYVPESDASARVTQIL

SEQ ID NO: 339 - protein, 58 aa, synthetic construct
  VARIANT 1: note = X is Ile or Val
  VARIANT 2: note = X is Leu or Ile
  VARIANT 3: note = X is Arg or Lys
  VARIANT 6: note = X is Val, Ile, or Thr
  VARIANT 8: note = X is Pro, Gln, or Thr
  VARIANT 9: note = X is Gly, Ala, or Ser
  VARIANT 12: note = X is Ala or Val
  VARIANT 13: note = X is Val or Thr
  VARIANT 30: note = X is Ser or Ala
  VARIANT 35: note = X is Val or Ile
  VARIANT 36: note = X is Pro, Thr, Ala, or Gln
  VARIANT 37: note = X is Glu or Asp
  VARIANT 38: note = X is Ser, Thr, or Asp
  VARIANT 41: note = X is Ser or Ala
  VARIANT 42: note = X is Ala, Gln, Arg, or Lys
  VARIANT 43: note = X is Arg, Lys, or any other amino acid
  VARIANT 45: note = X is Thr or Met
  VARIANT 46: note = X is Gln, Ala, Thr, or Gly
  VARIANT 47: note = X is Ile, Leu, or Val
  VARIANT 49..58: note = Each copy of Lys at positions 49 to 58 is either present or absent such that the number of copies of Lys ranges from 2 to 10
XXXRHXGXXEGXXQWMNRLIAFASRGNHVXPTHYXXXXDAXXXVXXXLKKKKKKKKKK

SEQ ID NO: 340 - protein, 51 aa, synthetic construct
  VARIANT 1: note = X is Ile or Val
  VARIANT 2: note = X is Leu or Ile
  VARIANT 3: note = X is Arg or Lys
  VARIANT 6: note = X is Val, Ile, or Thr
  VARIANT 8: note = X is Pro, Gln, or Thr
  VARIANT 9: note = X is Gly, Ala, or Ser
  VARIANT 12: note = X is Ala or Val
  VARIANT 13: note = X is Val or Thr
  VARIANT 30: note = X is Ser or Ala
  VARIANT 35: note = X is Val or Ile
  VARIANT 36: note = X is Pro, Thr, Ala, or Gln
  VARIANT 37: note = X is Glu or Asp
  VARIANT 38: note = X is Ser, Thr, or Asp
  VARIANT 41: note = X is Ser or Ala
  VARIANT 42: note = X is Ala, Gln, Arg, or Lys
  VARIANT 43: note = X is Arg, Lys, or any other amino acid
  VARIANT 45: note = X is Thr or Met
  VARIANT 46: note = X is Gln, Ala, Thr, or Gly
  VARIANT 47: note = X is Ile, Leu, or Val
XXXRHXGXXEGXXQWMNRLIAFASRGNHVXPTHYXXXXDAXXXVXXXLKKK

SEQ ID NO: 341 - protein, 50 aa, synthetic construct
LRRHVGPGEGAVQWMNRLIAFASRGNHVSPTHYVPESDASARVTQILKKK

SEQ ID NO: 342 - protein, 48 aa, synthetic construct
  VARIANT 1: note = X is Ile or Val
  VARIANT 2: note = X is Leu or Ile
  VARIANT 3: note = X is Arg or Lys
  VARIANT 6: note = X is Val, Ile, or Thr
  VARIANT 8: note = X is Pro, Gln, or Thr
  VARIANT 9: note = X is Gly, Ala, or Ser
  VARIANT 12: note = X is Ala or Val
  VARIANT 13: note = X is Val or Thr
  VARIANT 30: note = X is Ser or Ala
  VARIANT 35: note = X is Val or Ile
  VARIANT 36: note = X is Pro, Thr, Ala, or Gln
  VARIANT 37: note = X is Glu or Asp
  VARIANT 38: note = X is Ser, Thr, or Asp
  VARIANT 41: note = X is Ser or Ala
  VARIANT 42: note = X is Ala, Gln, Arg, or Lys
  VARIANT 43: note = X is Arg, Lys, or any other amino acid
  VARIANT 45: note = X is Thr or Met
  VARIANT 46: note = X is Gln, Ala, Thr, or Gly
  VARIANT 47: note = X is Ile, Leu, or Val
XXXRHXGXXEGXXQWMNRLIAFASRGNHVXPTHYXXXXDAXXXVXXXL

SEQ ID NO: 343 - protein, 48 aa, synthetic construct
  VARIANT 1: note = X is Ile or Val
  VARIANT 6: note = X is Val, Ile, or Thr
  VARIANT 8: note = X is Pro, Gln, or Thr
  VARIANT 9: note = X is Gly, Ala, or Ser
  VARIANT 13: note = X can be any naturally occurring amino acid
  VARIANT 30: note = X is Ser or Ala
  VARIANT 36: note = X is Pro, Thr, Ala, or Gln
  VARIANT 38: note = X is Ser, Thr, or Asp
  VARIANT 41: note = X is Ser or Ala
  VARIANT 42: note = X is Ala, Gln, Arg, or Lys
  VARIANT 43: note = X is Arg, Lys, or any other amino acid
  VARIANT 46: note = X is Gln, Ala, Thr, or Gly
  VARIANT 47: note = X is Ile, Leu, or Val
XLRRHXGXXEGAXQWMNRLIAFASRGNHVXPTHYVXEXDAXXXVTXXL

SEQ ID NO: 344 - protein, 48 aa, synthetic construct
ILRRHVGPGEGAVQWMNRLIAFASRGNHVSPTHYVPESDAAARVTAIL

SEQ ID NO: 345 - skipped

SEQ ID NO: 346 - protein, 48 aa, synthetic construct
ILRRHVGPGEGAVQWMNRLIAFASRGNHVAPTHYVTESDASQRVTQLL

SEQ ID NO: 347 - protein, 48 aa, synthetic construct
ILRRHVGQGEGAVQWMNRLIAFASRGNHVAPTHYVAESDASQRVTQVL

SEQ ID NO: 348 - protein, 48 aa, synthetic construct
ILRRHVGPGEGAVQWMNRLIAFASRGNHVSPTHYVPESDAAARVTALL

SEQ ID NO: 349 - protein, 48 aa, synthetic construct
VLRRHIGPGEGAVQWMNRLIAFASRGNHVSPTHYVPETDASAKVTQLL

SEQ ID NO: 350 - protein, 48 aa, synthetic construct
  VARIANT 43: note = X can be any naturally occurring amino acid
ILRRHVGPAEGATQWMNRLIAFASRGNHVSPTHYVPETDASRXVTTIL

SEQ ID NO: 351 - protein, 48 aa, synthetic construct
IIKRHTGTSEGVTQWMNRLIAFASRGNHVSPTHYIQDDDASKRVMGIL

SEQ ID NO: 352 - protein, 52 aa, synthetic construct; identical to SEQ ID NO: 314
  VARIANT 1: note = X is Gly, Pro, or Ser
  VARIANT 2: note = X is Thr, Asn, Gln, His, or Ser
  VARIANT 3: note = X is Glu, Thr, or Asp
  VARIANT 11: note = X is Lyr or Arg
  VARIANT 15: note = X is Leu or Ile
  VARIANT 16: note = X is Glu, Ala, Ser, or Gln
  VARIANT 17: note = X is Gln, Thr, Tyr, Phe, Val, or Leu
  VARIANT 18: note = X is Ile or Leu
  VARIANT 36: note = X is Leu or Ile
  VARIANT 38: note = X is Ala, Lys, or Ser
  VARIANT 39: note = X is Lys, Gln, or Ala
  VARIANT 41: note = X is Thr, Arg, Val, or Ser
  VARIANT 42: note = X is Gly or Ser
  VARIANT 43..52: note = Each copy of Lys at positions 43 to 52 is either present or absent such that the number of copies of Lys ranges from 2 to 10

SEQ ID NO: 353 - protein, 45 aa, synthetic construct; identical to SEQ ID NO: 315
  VARIANT 1: note = X is Gly, Pro, or Ser
  VARIANT 2: note = X is Thr, Asn, Gln, His, or Ser
  VARIANT 3: note = X is Glu, Thr, or Asp
  VARIANT 11: note = X is Lyr or Arg
  VARIANT 15: note = X is Leu or Ile
  VARIANT 16: note = X is Glu, Ala, Ser, or Gln
  VARIANT 17: note = X is Gln, Thr, Tyr, Phe, Val, or Leu
  VARIANT 18: note = X is Ile or Leu
  VARIANT 36: note = X is Leu or Ile
  VARIANT 38: note = X is Ala, Lys, or Ser
  VARIANT 39: note = X is Lys, Gln, or Ala
  VARIANT 41: note = X is Thr, Arg, Val, or Ser
  VARIANT 42: note = X is Gly or Ser

SEQ ID NO: 354 - protein, 15 aa, synthetic construct
  VARIANT 5: note = X is Val or Ile
  VARIANT 6: note = X is Pro, Thr, Ala, or Gln
  VARIANT 7: note = X is Glu or Asp
  VARIANT 8: note = X is Ser, Thr, or Asp
  VARIANT 11: note = X is Ser or Ala
  VARIANT 12: note = X is Ala, Gln, Arg, or Lys
  VARIANT 13: note = X is Arg, Lys, or any other amino acid
  VARIANT 15: note = X is Thr or Met
PTHYXXXXDAXXXVX

SEQ ID NO: 355 - protein, 15 aa, synthetic construct
  VARIANT 6: note = X is Pro, Thr, or Ala
  VARIANT 8: note = X is Ser or Thr
  VARIANT 11: note = X is Ser or Ala
  VARIANT 12: note = X is Ala, Gln, or Arg
  VARIANT 13: note = X is Arg or Lys
PTHYVXEXDAXXXVT

SEQ ID NO: 356 - protein, 45 aa, synthetic construct
RHVGPGEGAVQWMNRLIAFASRGNHVSPTHYVPESDASARVTQIL

SEQ ID NO: 357 - protein, 45 aa, synthetic construct
ILRRHVGPGEGAVQWMNRLIAFASRGNHVSPTHYVPESDASARVT

SEQ ID NO: 358 - protein, 33 aa, synthetic construct
HSKKKCDELAAKLTGLGLNAVAYYRGLDVSVIP

SEQ ID NO: 359 - protein, 19 aa, synthetic construct
DELAAKLTGLGLNAVAYYR

SEQ ID NO: 360 - protein, 19 aa, synthetic construct
DELAAKLVALGINAVAYYR

SEQ ID NO: 361 - protein, 19 aa, synthetic construct
DELAAKLSGLGLNAVAYYR

SEQ ID NO: 362 - protein, 19 aa, synthetic construct
DELAAALRGMGLNAVAYYR

SEQ ID NO: 363 - protein, 19 aa, synthetic construct
DELAAALRGMGVNAVAYYR

SEQ ID NO: 364 - protein, 19 aa, synthetic construct
DELASKLRGMGLNAVAYYR

SEQ ID NO: 365 - protein, 19 aa, synthetic construct
DELAAKLRGMGLNAVAYYR

SEQ ID NO: 366 - protein, 33 aa, synthetic construct
  VARIANT 11: note = X is Ala or Ser
  VARIANT 12: note = X is Lys or Ala
  VARIANT 14: note = X is Val, Ser, Arg, or Thr
  VARIANT 15: note = X is Ala or Gly
  VARIANT 16: note = X is Leu or Met
  VARIANT 18: note = X is Ile, Leu, or Val
  VARIANT 31: note = X is Val or Ile
HSKKKCDELAXXLXXXGXNAVAYYRGLDVSXIP

SEQ ID NO: 367 - protein, 33 aa, synthetic construct
  VARIANT 14: note = X is Val, Ser, Arg, or Thr
  VARIANT 15: note = X is Ala or Gly
  VARIANT 16: note = X is Leu or Met
  VARIANT 18: note = X is Ile, Leu, or Val
HSKKKCDELAAKLXXXGXNAVAYYRGLDVSVIP

SEQ ID NO: 368 - protein, 33 aa, synthetic construct
  VARIANT 18: note = X is Ile, Leu, or Val
  VARIANT 31: note = X is Val or Ile
HSKKKCDELAAALRGMGXNAVAYYRGLDVSXIP

SEQ ID NO: 369 - protein, 33 aa, synthetic construct
HSKKKCDELAAKLRGMGLNAVAYYRGLDVSVIP

SEQ ID NO: 370 - protein, 42 aa, synthetic construct
KGGRHLIFCHSKKKCDELAAKLTGLGLNAVAYYRGLDVSVIP

SEQ ID NO: 371 - protein, 42 aa, synthetic construct
  VARIANT 20: note = X is Ala or Ser
  VARIANT 21: note = X is Lys or Ala
  VARIANT 23: note = X is Val, Ser, Arg, or Thr
  VARIANT 24: note = X is Ala or Gly
  VARIANT 25: note = X is Leu or Met
  VARIANT 27: note = X is Ile, Leu, or Val
  VARIANT 40: note = X is Val or Ile
KGGRHLIFCHSKKKCDELAXXLXXXGXNAVAYYRGLDVSXIP

SEQ ID NO: 372 - protein, 42 aa, synthetic construct
  VARIANT 23: note = X is Val, Ser, Arg, or Thr
  VARIANT 24: note = X is Ala or Gly
  VARIANT 25: note = X is Leu or Met
  VARIANT 27: note = X is Ile, Leu, or Val
KGGRHLIFCHSKKKCDELAAKLXXXGXNAVAYYRGLDVSVIP

SEQ ID NO: 373 - protein, 42 aa, synthetic construct
  VARIANT 27: note = X is Ile, Leu, or Val
  VARIANT 40: note = X is Val or Ile
KGGRHLIFCHSKKKCDELAAALRGMGXNAVAYYRGLDVSXIP

SEQ ID NO: 374 - protein, 42 aa, synthetic construct
KGGRHLIFCHSKKKCDELAAKLRGMGLNAVAYYRGLDVSVIP

SEQ ID NO: 375 - protein, 65 aa, synthetic construct
  VARIANT 19: note = X is Glu, Thr, or Val
  VARIANT 58: note = X is Ser, Ala, or Pro
TPIDTTIMAKNEVFCVDPXKGGRKPARLIVYPDLGVRVCEKMALYDVVQKLPQAVMGXSYGFQYS

SEQ ID NO: 376 - protein, 65 aa, synthetic construct
TPIDTTIMAKNEVFCVDPEKGGRKPARLIVYPDLGVRVCEKMALYDVVQKLPQAVMGSSYGFQYS

SEQ ID NO: 377 - protein, 65 aa, synthetic construct
TPIDTTIMAKNEVFCVDPTKGGRKPARLIVYPDLGVRVCEKMALYDVVQKLPQAVMGASYGFQYS

SEQ ID NO: 378 - protein, 65 aa, synthetic construct
TPIDTTIMAKNEVFCVDPVKGGRKPARLIVYPDLGVRVCEKMALYDVVQKLPQAVMGPSYGFQYS

SEQ ID NO: 379 - protein, 240 aa, synthetic construct
  VARIANT 100: note = X is Thr, Val, or Gln
  VARIANT 148: note = X is Thr, Asn, Gln, His, or Ser
  VARIANT 162: note = X is Gle, Ser, or Ala
  VARIANT 163: note = X is Val, Thr, Tyr, Phe, or Leu
  VARIANT 208: note = X is Ser, Gln, or Thr
SYQVGYLHAPTGSGKSTKVPAAYAAQGYKVLVLNPSVAATLGFGAYMSKAHGIDPNIRTGVRTVTTGAPITYSTYGKFLADGGCSGGAYDIIICDECHSXDATTILGIGTVLDQAETAGARLVVLATATPPGSVTVPHPNIEEVALGXEGEIPFYGKAIPLXXIKGGRHLIFCHSKKKCDELAAKLRGMGLNAVAYYRGLDVSVIPTXGDVVVVATDALMTGYTGDFDSVIDCNVAVTQT

SEQ ID NO: 380 - protein, 240 aa, synthetic construct
SYQVGYLHAPTGSGKSTKVPAAYAAQGYKVLVLNPSVAATLGFGAYMSKAHGIDPNIRTGVRTVTTGAPITYSTYGKFLADGGCSGGAYDIIICDECHSTDATTILGIGTVLDQAETAGARLVVLATATPPGSVTVPHPNIEEVALGTEGEIPFYGKAIPLEVIKGGRHLIFCHSKKKCDELAAKLRGMGLNAVAYYRGLDVSVIPTSGDVVVVATDALMTGYTGDFDSVIDCNVAVTQT

SEQ ID NO: 381 - protein, 240 aa, synthetic construct
SYQVGYLHAPTGSGKSTKVPAAYAAQGYKVLVLNPSVAATLGFGAYMSKAHGIDPNIRTGVRTVTTGAPITYSTYGKFLADGGCSGGAYDIIICDECHSVDATTILGIGTVLDQAETAGARLVVLATATPPGSVTVPHPNIEEVALGNEGEIPFYGKAIPLSTIKGGRHLIFCHSKKKCDELAAKLRGMGLNAVAYYRGLDVSVIPTQGDVVVVATDALMTGYTGDFDSVIDCNVAVTQT

SEQ ID NO: 382 - protein, 228 aa, synthetic construct
LHAPTGSGKSTKVPAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGFTGDFDSVIDCN

SEQ ID NO: 383 - protein, 465 aa, synthetic construct
SFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRI

SEQ ID NO: 384 - protein, 156 aa, synthetic construct
KFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFL

SEQ ID NO: 385 - protein, 85 aa, synthetic construct
KSTKVPAAYAAQGYKVLVLNPSVAATLGFGAYMSKGVYLLPRRGPRLGVRATRKTSERSQPRGRRQPIPKARRSEGRSWAQPGYP

SEQ ID NO: 386 - protein, 85 aa, synthetic construct
GVYLLPRRGPRLGVRATRKTSERSQPRGRRQPIPKARRSEGRSWAQPGYPKSTKVPAAYAAQGYKVLVLNPSVAATLGFGAYMSK

SEQ ID NO: 387 - protein, 108 aa, synthetic construct
KSTKVPAAYAAQGYKVLVLNPSVAATLGFGAYMSKGVYLLPRRGPRLGVRATRKTSERSQPRGRRQPIPKARRSEGRSWAQPGYPDVVVVATDALMTGFTGDFDSVID

SEQ ID NO: 388 - protein, 135 aa, synthetic construct
KSTKVPAAYAAQGYKVLVLNPSVAATLGFGAYMSKGVYLLPRRGPRLGVRATRKTSERSQPRGRRQPIPKARRSEGRSWAQPGYPDVVVVATDALMTGFTGDFDSVIDLEQIKGGRHLIFCHSKKKCDELAAKLT

SEQ ID NO: 389 - protein, 170 aa, synthetic construct
KSTKVPAAYAAQGYKVLVLNPSVAATLGFGAYMSKGVYLLPRRGPRLGVRATRKTSERSQPRGRRQPIPKARRSEGRSWAQPGYPDVVVVATDALMTGFTGDFDSVIDLEQIKGGRHLIFCHSKKKCDELAAKLTILRRHVGPGEGAVQWMNRLIAFASRGNHVSPTHYV

SEQ ID NO: 390 - protein, 52 aa, synthetic construct
  VARIANT 43..52: note = Each copy of Lys at positions 43 to 52 is either present or absent such that the number of copies of Lys ranges from 2 to 10
GTEGEIPFYGKAIPLEQIKGGRHLIFCHSKKKCDELAAKLTGKKKKKKKKKK

SEQ ID NO: 391 - protein, 45 aa, synthetic construct
GTEGEIPFYGKAIPLEQIKGGRHLIFCHSKKKCDELAAKLTGKKK

SEQ ID NO: 392 - protein, 55 aa, synthetic construct
  VARIANT 46..55: note = Each copy of Lys at positions 46 to 55 is either present or absent such that the number of copies of Lys ranges from 2 to 10
LNAVAYYRGLDVSVIPTSGDVVVVATDALMTGFTGDFDSVIDCNVKKKKKKKKKK

SEQ ID NO: 393 - protein, 58 aa, synthetic construct
  VARIANT 49..58: note = Each copy of Lys at positions 49 to 58 is either present or absent such that the number of copies of Lys ranges from 2 to 10
ILRRHVGPGEGAVQWMNRLIAFASRGNHVSPTHYVPESDASARVTQILKKKKKKKKKK

SEQ ID NO: 394 - protein, 51 aa, synthetic construct
ILRRHVGPGEGAVQWMNRLIAFASRGNHVSPTHYVPESDASARVTQILKKK

SEQ ID NO: 395 - protein, 38 aa, synthetic construct
KSTKVPAAYAAQGYKVLVLNPSVAATLGFGAYMSKKKK

SEQ ID NO: 396 - protein, 44 aa, synthetic construct
  VARIANT 35..44: note = Each copy of Lys at positions 35 to 44 is either present or absent such that the number of copies of Lys ranges from 2 to 10
KSTKVPAAYAAQGYKVLVLNPSVAATLGFGAYMSKKKKKKKKKK

SEQ ID NO: 397 - DNA, 24 bp, synthetic construct
  source 1..24: mol_type = other DNA
tcgtcgttttgtcgttttgtcgtt